(19) United States
(12) Patent Application Publication
(10) Pub. No.: US 2012/0328569 A1
(43) Pub. Date: Dec. 27, 2012

(54) INHIBITORS OF HEPATITIS C VIRUS NS5B POLYMERASE

(76) Inventors: Casey Cameron McComas, Phoenixville, PA (US); Nigel J. Liverton, Harleysville, PA (US); Richard Soll, Shanghai (CN); Peng Li, Shanghai (CN); Xuanjia Peng, Shanghai (CN); Hao Wu, Shanghai (CN); Frank Narjes, Ariccia (IT); Joerg Habermann, Munchen (DE); Uwe Koch, Dortmund (DE); Shilan Liu, Shanghai (CN)

(21) Appl. No.: 13/582,240

(22) PCT Filed: Mar. 2, 2011
(86) PCT No.: PCT/CN11/00332

§ 371 Date: Aug. 31, 2012

Related U.S. Application Data
(60) Provisional application No. 61/320,463, filed on Apr. 2, 2010.

(30) Foreign Application Priority Data
Mar. 2, 2010 (CN) PCT/CN2010/070831
Dec. 27, 2010 (CN) PCT/CN2010/080332

(51) Int. Cl. A61K 31/343; A61K 38/21; A61K 31/7056; C07D 407/12; A61K 31/4025; C07D 405/12; A61K 31/4178; C07D 413/10; A61K 31/422; C07D 417/10; A61K 31/428; A61K 31/427; A61K 31/4245; A61K 31/423; A61K 31/41; C07D 471/04; A61K 31/437; C07D 513/04; C07D 498/04; A61K 31/517; C07D 487/04; A61K 31/4985; A61K 31/519; A61K 31/549; A61K 31/5377; A61P 31/14; C07D 307/84

(52) U.S. Cl. 424/85.7; 549/468; 514/469; 514/43; 548/561; 514/427; 548/311.4; 514/389; 548/231; 514/376; 548/159; 514/367; 548/204; 514/365; 548/145; 514/364; 548/241; 514/379; 548/253; 514/382; 546/121; 514/300; 546/114; 514/301; 514/302; 546/115; 544/283; 514/266.24; 544/350; 514/249; 544/279; 514/264.1; 544/13; 514/223.2; 544/153; 514/233.5; 514/210.18

(57) ABSTRACT

Disclosed are compounds of formula (I) that are used as hepatitis C virus (HCV) NS5B polymerase inhibitors, the synthesis of such compounds, and the use of such compounds for inhibiting HCV NS5B polymerase activity, for treating or preventing HCV infections and for inhibiting HCV viral replication and/or viral production in a cell-based system.

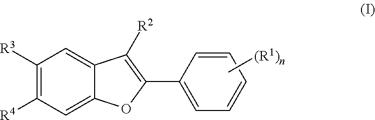

### FIELD OF THE INVENTION

The present disclosure relates to antiviral compounds that are useful as inhibitors of the hepatitis C virus (HCV) NS5B (non-structural protein 5B) polymerase, compositions comprising such compounds, the use of such compounds for treating HCV infection and/or reducing the likelihood or severity of symptoms of HCV infection, methods for inhibiting the function of the NS5B polymerase, and methods for inhibiting HCV viral replication and/or viral production.

### BACKGROUND OF THE INVENTION

Hepatitis C virus (HCV) infection is a major health problem that leads to chronic liver disease, such as cirrhosis and hepatocellular carcinoma, in a substantial number of infected individuals. Current treatments for HCV infection include immunotherapy with recombinant interferon-α alone or in combination with the nucleoside analog ribavirin.

Several virally-encoded enzymes are putative targets for therapeutic intervention, including a metalloprotease (NS2-3), a serine protease (NS3, amino acid residues 1-180), a helicase (NS3, full length), an NS3 protease cofactor (NS4A), a membrane protein (NS4B), a zinc metalloprotein (NS5A) and an RNA-dependent RNA polymerase (NS5B).

One identified target for therapeutic intervention is HCV NS5B polymerase. Sven-Erik Behrens et al., Identification and properties of the RNA-dependent RNA polymerase of heptatitis C virus, 15(1) EMBO J. 12-22 (1996). Antagonists of NS5B activity are inhibitors of HCV replication. Steven S. Carroll et al., Inhibition of Hepatitis C Virus RNA Replication by 2′-Modified Nucleoside Analogs, 278(14) J. BIOL. CHEM. 11979-84 (2003).

There is a clear and long-felt need to develop effective therapeutics for treatment of HCV infection. Specifically, there is a need to develop compounds that selectively inhibit HCV viral replication and that would be useful for treating HCV-infected patients.

### SUMMARY OF THE INVENTION

The present disclosure relates to novel compounds of formula (I) and/or pharmaceutically acceptable salts thereof. These compounds are useful, either as compounds or their pharmaceutically acceptable salts (when appropriate), in the inhibition of HCV (hepatitis C virus) NS5B (non-structural 5B) polymerase, the prevention or treatment of one or more of the symptoms of HCV infection, the inhibition of HCV viral replication and/or HCV viral production, and/or as pharmaceutical composition ingredients. As pharmaceutical composition ingredients, these compounds and their salts may be the primary active therapeutic agent, and, when appropriate, may be combined with other therapeutic agents including but not limited to other HCV antivirals, anti-infectives, immunomodulators, antibiotics or vaccines, as well as the present Standard of Care treatment options for HCV.

More particularly, the present disclosure relates to a compound of formula (I):

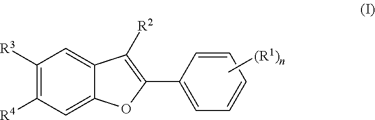

or a pharmaceutically acceptable salt thereof, wherein:
each R¹ is independently selected from the group consisting of halo, C₁-C₆ alkyl, —O—(C₁-C₆ alkyl), —O—(C₁-C₆ haloalkyl) and —CN;

n is 0, 1, 2, 3 or 4;

R² is C(O)NR^{a}R^{b};
R^{a} and R^{b} are independently selected from the group consisting of hydrogen, C₁-C₆ alkyl, O(C₁-C₆ alkyl) and 5- or 6-membered monocyclic aromatic rings with 0, 1, 2, 3 or 4 heteroatom ring atoms independently selected from the group consisting of N, O or S;

R³ is ArA, —C≡C-phenyl or a 15- or 16-membered tetracyclic ring system,
wherein said 15- or 16-membered tetracyclic ring system is substituted by 0, 1 or 2 substitutents independently selected from C₁-C₆ alkyl, phenyl, C₃-C₇ cycloalkyl or 6-membered heteroaryl, and
wherein ArA is an aromatic ring system selected from the group consisting of:
i) 5- or 6-membered monocyclic rings with 0, 1, 2, 3 or 4 heteroatom ring atoms independently selected from the group consisting of N, O or S, and
ii) 8-, 9- or 10-membered bicyclic rings with 0, 1, 2, 3 or 4 heteroatom ring atoms independently selected from the group consisting of N, O or S, and
wherein said ArA is substituted by 0, 1, 2, 3 or 4 substitutents R^{c};
each R^{c} is independently selected from the group consisting of:
   a) halogen,
   b) OH
   c) C₁-C₆ alkyl,
   d) O(C₁-C₆ alkyl),
   e) CN,
   f) (CH₂)₀₋₃-ArB, wherein each ArB is an independently selected aromatic ring system selected from the group consisting of:
      i) 5- or 6-membered monocyclic rings with 0, 1, 2, 3 or 4 heteroatom ring atoms independently selected from the group consisting of N, O or S, and
      ii) 8-, 9- or 10-membered bicyclic rings, which can be aromatic or non-aromatic, with 0, 1, 2, 3 or 4 heteroatom ring atoms independently selected from the group consisting of N, O or S,
   g) (CH₂)₀₋₃NR^{d}C(O)R^{e},
   h) (CH₂)₀₋₃NR^{d}SO₂R^{e},
   i) (CH₂)₀₋₃C(O)NR^{d}R^{e},
   j) (CH₂)₀₋₃SO₂R^{e},
   k) —OSO₂(C₁-C₆ alkyl), and
   l) C₂-C₆ alkynyl
   wherein each R^{c} c) C₁-C₆ alkyl, d) O(C₁-C₆ alkyl), and f) (CH₂)₀₋₃-ArB is substituted by 0, 1, 2, 3 or 4 substituents R^{f}; or
   wherein any 2 R^{c} groups on adjacent ring carbon atoms can join to form a group selected from —OC(O)—N—, —OCH₂CH₂O—, —OCH₂O—, —OCH₂CH₂—,
each R^{d} is independently selected from the group consisting of hydrogen and C₁₋₆alkyl;
each R^{e} is independently selected from the group consisting of hydrogen, C₁₋₆alkyl, OC₁₋₆alkyl and 5- or 6-membered monocyclic rings with 0, 1, 2, 3 or 4 heteroatom ring atoms independently selected from the group consisting of N, O or S, wherein each R^{e}C₁₋₆alkyl, OC₁₋₆alkyl and 5- or 6-membered monocyclic rings is substituted by 0, 1, 2, 3 or 4 substituents independently selected from the group consisting of C₁-C₆ alkyl, O(C₁-C₆ alkyl), halogen and OH;
each R^{f} is independently selected from the group consisting of:
   a) halogen,
   b) C₁-C₆ alkyl,
   c) O(C₁-C₆ alkyl),
   d) CN,
   e) N(R^{q})₂,
   f) OH,
   g) C(O)H,
   h) NHC(O)R^{s},
   i) NHS(O)₂R^{s},
   j) C(O)NHR^{q},
   k) C(O)OR^{q},
   l) OS(O)₂(C₁-C₆ alkyl),
   m) (CH₂)₀₋₃-ArC, wherein each ArC is an independently selected aromatic ring system selected from the group consisting of:
      i) 5- or 6-membered monocyclic rings with 0, 1, 2, 3 or 4 heteroatom ring atoms independently selected from the group consisting of N, O or S, and
      ii) 8-, 9- or 10-membered bicyclic rings with 0, 1, 2, 3 or 4 heteroatom ring atoms independently selected from the group consisting of N, O or S,
   wherein each R^{f}: b) C₁-C₆ alkyl, c) O(C₁-C₆ alkyl), and m) (CH₂)₀₋₃-ArC is substituted by 0, 1, 2, 3 or 4 substituents R⁹;
each R⁹ is independently selected from the group consisting of halogen, N(R^{q})₂, CN, C₁₋₆alkyl, O(C₁-C₆ alkyl), CF₃ and C(O)OH;

R⁴ is selected from the group consisting of NR^{h}Rⁱ and 5- or 6-membered monocyclic rings with 0, 1, 2, 3 or 4 heteroatom ring atoms independently selected from the group consisting of N, O or S, and
R^{h} is selected from the group consisting of:
   a) hydrogen,
   b) C₁₋₆alkyl,
   c) C(O)O(C₁₋₆alkyl), and
   d) SO₂R^{J};
   R^{j} is selected from the group consisting of C₁₋₆alkyl, C₆₋₁₀ aryl, C₃₋₇ cycloalkyl and NR^{X}R^{y}, where R^{x} and R^{y} are independently selected from the group consisting of hydrogen and C₁₋₆alkyl;
Rⁱ is selected from the group consisting of:
   a) C₁₋₆alkyl,
   b) C₂₋₆alkenyl,
   c) C₂₋₆alkynyl,
   d) (CH₂)₀₋₃(C₃₋₈cycloalkyl),
   e) (CH₂)₀₋₃(C₃₋₈cycloalkenyl),
   f) C(O)C₁₋₆alkyl, and
   g) heterocyclyl,
   wherein Rⁱ is substituted by 0, 1, 2, 3 or 4 R^{k} groups;
   each R^{k} is independently selected from the group consisting of:
      a) OR^{L},
      b) halogen,
      c) CN,
      d) NR^{m}Rⁿ,
      e) OC(O)C₁₋₆alkyl,
      f) C(O)OC₁₋₆alkyl,
      g) —P(O)(O—C₁₋₆alkyl)₂,
      h) —P(O)(OH)(O—C₁₋₆alkyl),
      j) —P(O)(OH)₂,
      k) —C(O)C(C₁₋₆alkyl)-NHC(O)—C₁₋₆alkyl,
      l) —NHC(O)C(C₁₋₆alkyl)-NHC(O)—C₁₋₆alkyl,
      m) —C(O)OH,
      n) (CH₂)₀₋₃-ArD, wherein each ArD is an independently selected aromatic ring system selected from the group consisting of:
         i) 5- or 6-membered monocyclic rings with 0, 1, 2, 3 or 4 heteroatom ring atoms independently selected from the group consisting of N, O or S, and
         ii) 8-, 9- or 10-membered bicyclic rings with 0, 1, 2, 3 or 4 heteroatom ring atoms independently selected from the group consisting of N, O or S,
      wherein each R^{k} e) OC(O)C₁₋₆alkyl, f) C(O)OC₁₋₆alkyl, and n) (CH₂)₀₋₃-ArD is substituted by 0, 1, 2, 3 or 4 R^{o} groups;
   R^{L} is selected from the group consisting of hydrogen, C₁₋₆alkyl and phenyl;
   R^{m} is selected from the group consisting of hydrogen, C₁₋₆alkyl, —CH₂CN and (CH₂)₀₋₃(phenyl);
   Rⁿ is selected from the group consisting of hydrogen, C₁₋₆alkyl, SO₂(C₁₋₆alkyl), —C(O)H, —C(O)OH, —C(O)O(C₁₋₆alkyl) and C(O)(C₁₋₆alkyl);
   or R^{m} and Rⁿ are taken together with the N to which they are attached to form a 5- to 7-membered ring substituted by 0, 1, 2 or 3 RP;
   each R^{o} is independently selected from the group consisting of halogen, C₁₋₆alkyl, OC₁₋₆alkyl and C(O)O(C₁₋₆alkyl);
   each R^{p} is independently selected from the group consisting of halogen, C₁₋₆alkyl, OC₁₋₆alkyl, oxo and C(O)O(C₁₋₆alkyl);
   each R^{q} is independently selected from the group consisting of H and C₁₋₆alkyl;
   each R^{s} is independently selected from the group consisting of C₁₋₆alkyl, heterocyclyl and C₆₋₁₀aryl, wherein said heterocyclyl group can be optionally substituted on a ring nitrogen or ring carbon atom with a —C(O)O—(C₁-C₆ alkyl) group; and
   each R^{t} is independently selected from the group consisting of C₁₋₆alkyl and C₆₋₁₀aryl;
   or R^{h} and Rⁱ are taken together with the N to which they are attached to form a 5- to 7-membered ring.

The present invention also includes pharmaceutical compositions containing a compound of the present invention and methods of preparing such pharmaceutical compositions. The present invention further includes methods of treating or reducing the likelihood or severity of HCV infection, methods for inhibiting the activity of the NS5B polymerase, and methods for inhibiting HCV viral replication and/or viral production.

Other embodiments, aspects and features of the present invention are either further described in or will be apparent from the ensuing description, examples and appended claims.

### DETAILED DESCRIPTION OF THE INVENTION

The present invention includes compounds of formula (I) above, and pharmaceutically acceptable salts thereof. The compounds of formula (I) are HCV NS5B polymerase inhibitors.

In a first embodiment of the invention, n is 1. In this embodiment, all other groups are as provided in the general formula above.

In a second embodiment of the invention, the compound is a compound of formula (Ia):

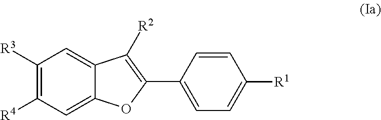

or a pharmaceutically acceptable salt thereof. In this embodiment, all other groups are as provided in the general formula above and/or in the first embodiment.

In a third embodiment of the invention, the compound is a compound of formula (Ib):

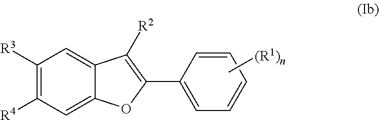

or a pharmaceutically acceptable salt thereof, wherein:

each R¹ is independently selected from the group consisting of halogens;

n is 0, 1, 2 or 3;

R² is C(O)NR^{a}R^{b};
R^{a} and R^{b} are independently selected from the group consisting of hydrogen, C₁-C₆ alkyl and O(C₁-C₆ alkyl);

R³ is ArA, wherein ArA is an aromatic ring system selected from the group consisting of:
i) 5- or 6-membered monocyclic rings, and
ii) 8-, 9- or 10-membered bicyclic rings, and
wherein said ArA is substituted by 0, 1, 2 or 3 substitutents R^{c};
each R^{e} is independently selected from the group consisting of:
   a) halogen,
   b) OH
   c) C₁-C₆ alkyl,
   d) O(C₁-C₆ alkyl),
   e) CN,
   f) (CH₂)₀₋₃-ArB, wherein each ArB is an independently selected aromatic ring system selected from the group consisting of:
      i) 5- or 6-membered monocyclic rings with 0, 1, 2, 3 or 4 heteroatom ring atoms independently selected from the group consisting of N, O or S, and
      ii) 8-, 9- or 10-membered bicyclic rings with 0, 1, 2, 3 or 4 heteroatom ring atoms independently selected from the group consisting of N, O or S,
   g) (CH₂)₀₋₃NR^{d}C(O)R^{e},
   h) (CH₂)₀₋₃NR^{d}SO₂R^{e},
   i) (CH₂)₀₋₃C(O)NR^{d}R^{e}, and
   j) (CH₂)₀₋₃SO₂R^{e},
   wherein each R^{e} c) C₁-C₆ alkyl, d) O(C₁-C₆ alkyl), and f) (CH₂)₀₋₃-ArB is substituted by 0, 1, 2 or 3 substituents R^{f};
each R^{d} is independently selected from the group consisting of hydrogen and C₁₋₆alkyl;
each R^{e} is independently selected from the group consisting of hydrogen, C₁₋₆alkyl, OC₁₋₆alkyl and 5- or 6-membered monocyclic rings with 0, 1, 2, 3 or 4 heteroatom ring atoms independently selected from the group consisting of N, O or S, wherein each R^{e}C₁₋₆alkyl, OC₁₋₆alkyl and 5- or 6-membered monocyclic rings is substituted by 0, 1, 2, 3 substituents independently selected from the group consisting of C₁-C₆ alkyl, O(C₁-C₆ alkyl), halogen and OH;
each R^{f} is independently selected from the group consisting of:
   a) halogen,
   b) C₁-C₆ alkyl,
   c) O(C₁-C₆ alkyl),
   d) CN,
   e) NH₂,
   f) (CH₂)₀₋₃—ArC, wherein each ArC is an independently selected aromatic ring system selected from the group consisting of:
      i) 5- or 6-membered monocyclic rings with 0, 1, 2, 3 or 4 heteroatom ring atoms independently selected from the group consisting of N, O or S, and
      ii) 8-, 9- or 10-membered bicyclic rings with 0, 1, 2, 3 or 4 heteroatom ring atoms independently selected from the group consisting of N, O or S,
   wherein each R^{f} b) C₁-C₆ alkyl, c) O(C₁-C₆ alkyl), and f) (CH₂)₀₋₃-ArC is substituted by 0, 1, 2 or 3 substituents R⁹;
each R⁹ is independently selected from the group consisting of halogen, CN, C₁₋₆alkyl, O(C₁-C₆ alkyl), CF₃ and C(O)OH;

R⁴ is selected from the group consisting of NR^{h}Rⁱ;
R^{h} is selected from the group consisting of:
   a) hydrogen,
   b) C₁₋₆alkyl,
   c) C(O)O(C₁₋₆alkyl), and
   d) SO₂R^{j};
   R^{j} is selected from the group consisting of C₁₋₆alkyl and NR^{X}R^{y}, where R^{x} and R^{y} are independently selected from the group consisting of hydrogen and C₁₋₆alkyl;
Rⁱ is selected from the group consisting of:
   a) C₁₋₆alkyl,
   b) C₂₋₆alkenyl,
   c) C₂₋₆alkynyl,
   d) (CH₂)₀₋₃(C₃₋₈cycloalkyl),
   e) (CH₂)₀₋₃(C₃₋₈cycloalkenyl), and
   f) C(O)C₁₋₆alkyl,
   wherein Rⁱ is substituted by 0, 1, 2, 3 or 4 R^{k};
   each R^{k} is independently selected from the group consisting of:
      a) OR^{L},
      b) halogen,
      c) CN,
      d) NR^{m}R^{h},
      e) OC(O)C₁₋₆alkyl,
      f) C(O)OC₁₋₆alkyl,
      g) (CH₂)₀₋₃-ArD, wherein each ArD is an independently selected aromatic ring system selected from the group consisting of:
         i) 5- or 6-membered monocyclic rings with 0, 1, 2, 3 or 4 heteroatom ring atoms independently selected from the group consisting of N, O or S, and
         ii) 8-, 9- or 10-membered bicyclic rings with 0, 1, 2, 3 or 4 heteroatom ring atoms independently selected from the group consisting of N, O or S,
      wherein each R^{k} e) OC(O)C₁₋₆alkyl, f) C(O)OC₁₋₆alkyl, and g) (CH₂)₀₋₃-ArD is substituted by 0, 1, 2 or 3 substituents R^{o},
   R^{L} is selected from the group consisting of hydrogen, C₁₋₆alkyl and phenyl;
   R^{m} is selected from the group consisting of hydrogen, C₁₋₆alkyl and (CH₂)₀₋₃(phenyl);
   Rⁿ is selected from the group consisting of hydrogen, C₁₋₆alkyl, SO₂(C₁₋₆alkyl) and C(O)(C₁₋₆alkyl);
   or R^{m} and Rⁿ are taken together with the N to which they are attached to form a 5- to 7-membered ring substituted by 0, 1, 2 or 3 R^{p};
   each R^{o} is independently selected from the group consisting of halogen, C₁₋₆alkyl, OC₁₋₆alkyl and C(O)O(C₁₋₆alkyl);
   each RP is independently selected from the group consisting of halogen, C₁₋₆alkyl, OC₁₋₆alkyl, oxo and C(O)O(C₁₋₆alkyl);
or R^{h} and Rⁱ are taken together with the N to which they are attached to form a 5- to 7-membered ring.

In a fourth embodiment of the invention, R¹ is selected from the group consisting of fluorine, bromine and chlorine. In a first aspect of this third embodiment, R¹ is fluorine. In all aspects of this embodiment, all other groups are as provided in the general formula above and/or in the first or second embodiments.

In a fifth embodiment of the invention, R^{a} is hydrogen. In this embodiment, all other groups are as provided in the general formula above and/or in the first through third embodiments.

In a sixth embodiment of the invention, R^{b} is selected from the group consisting of —CH₃ and —OCH₃. In this embodiment, all other groups are as provided in the general formula above and/or in the first through fourth embodiments.

In a seventh embodiment of the invention, ArA is phenyl or pyridyl. In a first aspect of this seventh embodiment, ArA is phenyl, which is optionally substituted with which is substituted by 0, 1, 2, 3 or 4 substitutents R^{c}. In a second aspect of this seventh embodiment, ArA is pyridyl, which is optionally substituted with which is substituted by 0, 1, 2, 3 or 4 substitutents R^{c} in these embodiments, all other groups are as provided in the general formula above and/or in the first through fifth embodiments.

In an eighth embodiment of the invention, each R^{c} is independently selected from the group consisting of a) fluorine, b) OH, c) C₁₋₃alkyl, d) OC₁₋₃alkyl, e) CN, f) (CH₂)₀₋₁-ArB, wherein ArB is independently selected from the group consisting of

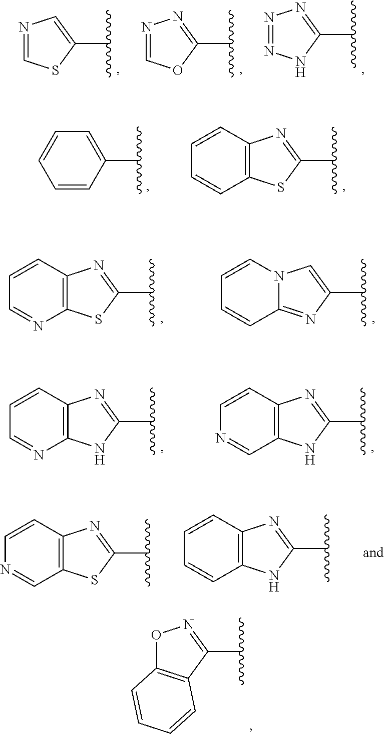

g) (CH₂)₀₋₁N(CH₃)SO₂CH₃, h) (CH₂)₀₋₁N(H)SO₂CH₃, i) (CH₂)₀₋₁N(CH₃)SO₂-phenyl, j) C(O)NHCH₃, k) (CH₂)₀₋₁N(H)C(O)CH₃, and l) (CH₂)₀₋₁N(H)C(O)phenyl. In a first aspect of this seventh embodiment each R^{c} is independently selected from the group consisting of

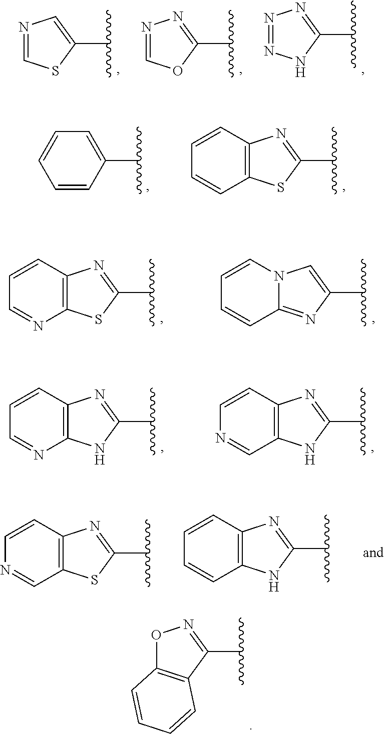

In all aspects of this embodiment, all other groups are as provided in the general formula above and/or in the first through sixth embodiments.

In a ninth embodiment of the invention, R^{h} is selected from hydrogen, CH₃ and SO₂CH₃. In a first aspect of this eighth embodiment, R^{h} is SO₂CH₃. In all aspects of this embodiment, all other groups are as provided in the general formula above and/or in the first through seventh embodiments.

In a tenth embodiment of the invention, Rⁱ is selected from the group consisting of C₁₋₆alkyl and C₂₋₆alkenyl. In this embodiment, all other groups are as provided in the general formula above and/or in the first through eighth embodiments.

In an eleventh embodiment of the invention, R^{k} is selected from the group consisting of a) OR^{L}, b) halogen, c) CN, d) NR^{m}Rⁿ, e) OC(O)C₁₋₆alkyl, and OC(O)OC₁₋₆alkyl. In this embodiment, all other groups are as provided in the general formula above and/or in the first through ninth embodiments.

In a twelfth embodiment of the invention, R^{L} is selected from the group consisting of C₁₋₆alkyl. In this embodiment, all other groups are as provided in the general formula above and/or in the first through tenth embodiments.

In a thirteenth embodiment of the invention, R^{m} is selected from the group consisting of hydrogen and C₁₋₆alkyl. In this embodiment, all other groups are as provided in the general formula above and/or in the first through eleventh embodiments.

In a fourteenth embodiment of the invention, Rⁿ is selected from the group consisting of C₁₋₆alkyl and SO₂(C₁₋₆alkyl). In this embodiment, all other groups are as provided in the general formula above and/or in the first through twelfth embodiments.

In a fifteenth embodiment of the invention, the compound is a compound of formula (Ic):

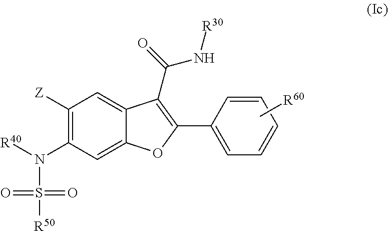

and pharmaceutically acceptable salts thereof,
wherein:

Z is a phenyl group which is substituted with one R¹⁰ group and optionally further substituted with R²⁰;

R¹⁰ is an 8- to 10-membered bicyclic heteroaryl group, wherein said 8- to 10-membered bicyclic heteroaryl group is optionally substituted with up to 4 groups, which can be the same or different, and are selected from halo, C₁-C₆ alkyl, —C(O)H, —(CH₂)ₜ—N(R⁷⁰)₂, —(CH₂)ₜ—OH, —(CH₂)ₜ—O—(C₁-C₆ alkyl), —CF₃, —NHC(O)-heterocyclyl, —NHC(O)—(C₁-C₆ alkyl), —C(O)NH—(C₁-C₆ alkyl), —C(O)OH, —C(O)O—(C₁-C₆ alkyl), —NHC(O)-aryl, —NHSO₂-aryl, —NHSO₂-alkyl, —O—SO₂-alkyl, —O—(C₁-C₆ alkyl) and —CN, wherein the heterocyclyl moiety of said —NHC(O)— heterocyclyl group can be optionally substituted on a ring carbon or ring nitrogen atom with a —C(O)O—(C₁-C₆ alkyl) group;

R²⁰ represents up to 4 optional substituents, which can be the same or different, and are selected from halo, 8- to 10-membered heteroaryl, C₁-C₆ alkyl, —O—(C₁-C₆ alkyl), —O—(CH₂)ₜ—OH, —O—(CH₂)ₜ-heterocyclyl, —O—(C₁-C₆ haloalkyl), —O—SO₂—(C₁-C₆ alkyl) and —CN;

R³⁰ is H or C₁-C₆ alkyl;

R⁴⁰ is selected from C₁-C₆ alkyl, C₁-C₆ haloalkyl, —(CH₂)ₜ—OH, —(CH₂)ₜ-heterocyclyl, —(CH₂)ₜ—N(R⁷⁰)₂, —(CH₂)ₜ—CN, —(CH₂)ₜ—NHC(O)OR³⁰ and —(CH₂)ₜ—NHC(O)R³⁰;

R⁵⁰ is C₁-C₆ alkyl, C₆-C₁₀ aryl or C₃-C₇ cycloalkyl;

R⁶⁰ represents up to 4 optional ring substituents, which can be the same or different, and are selected from halo, C₁-C₆ alkyl, —O—(C₁-C₆ alkyl), —O—(C₁-C₆ haloalkyl) and —CN;

each occurrence of R⁷⁰ is independently H or C₁-C₆ alkyl; and

each occurrence of t is independently an integer ranging from 0 to 6.

In a first aspect of this fifteenth embodiment, Z is:

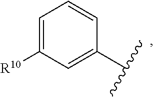

which can be optionally substituted on the depicted phenyl ring with one or two R²⁰ groups, which can be the same or different.

In a second aspect of this fifteenth embodiment, Z is selected from:

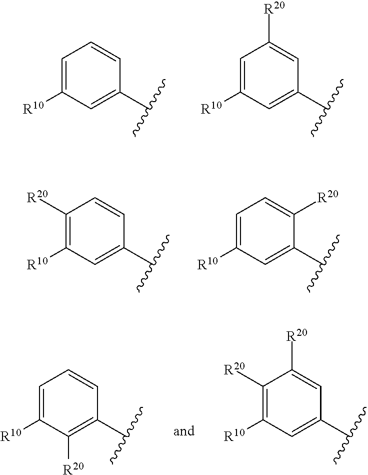

wherein each occurrence of R²⁰ is independently Cl, F, CN, —OCF₃ or —OCH₃.

In a third aspect of this fifteenth embodiment, Z is selected from:

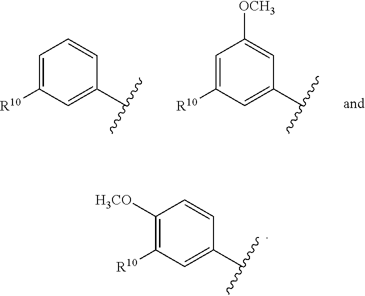

In a fourth aspect of this fifteenth embodiment of the present invention, R¹⁰ is selected from:

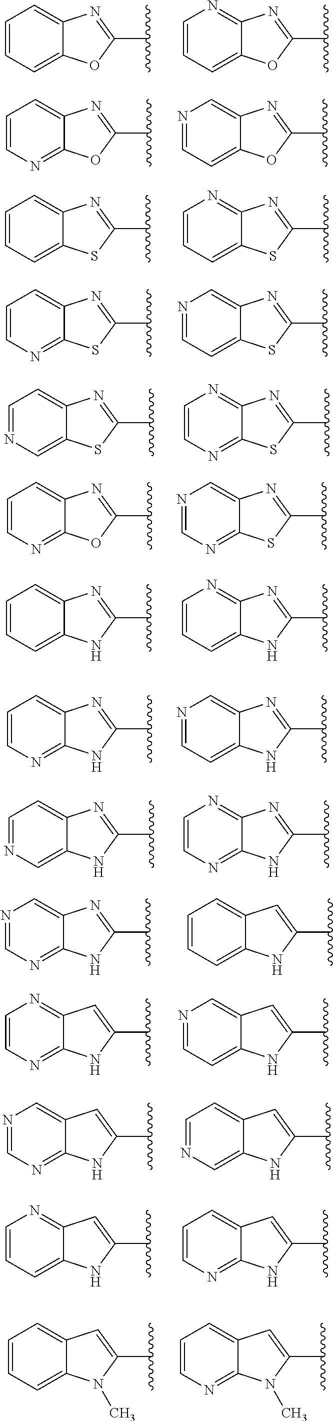

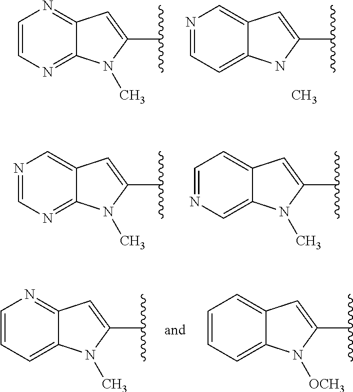

each of which can be optionally substituted as set forth above for the Compounds of Formula (Ic).

In a fifth aspect of this fifteenth embodiment, R¹⁰ is selected from:

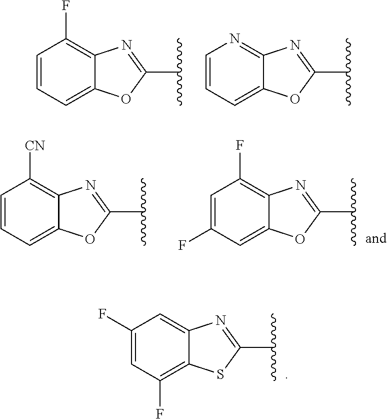

In a sixth aspect of this fifteenth embodiment, R¹⁰ is:

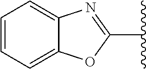

which can be optionally substituted as set forth above for the Compounds of Formula (Ic).

In a seventh aspect of this fifteenth embodiment, Z is selected from:

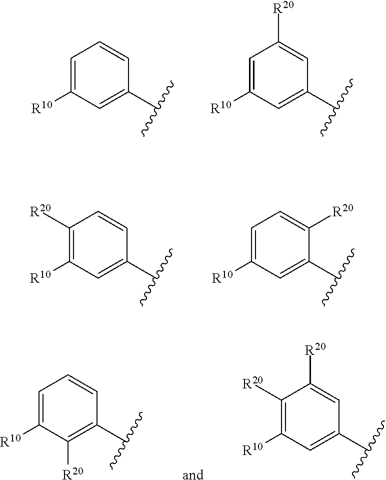

wherein each occurrence of R²⁰ is independently Cl, F, CN, —OCF₃ or —OCH₃; and R¹⁰ is selected from:

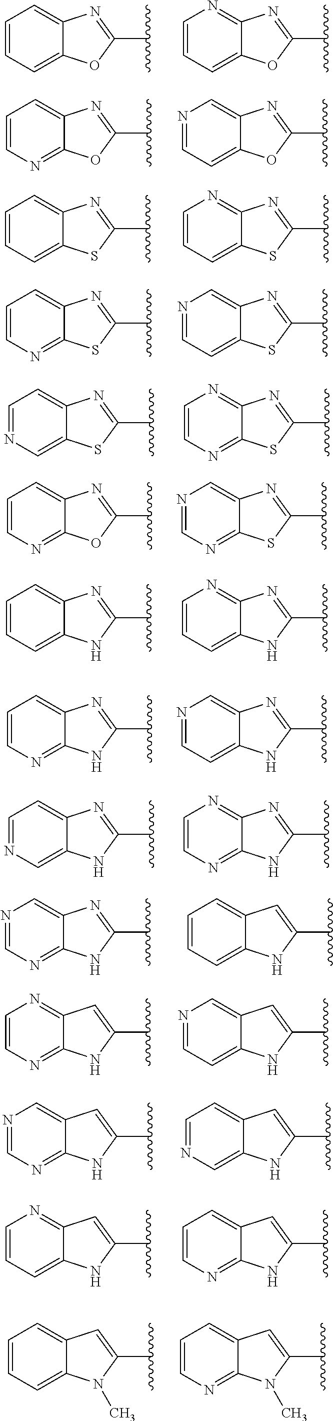

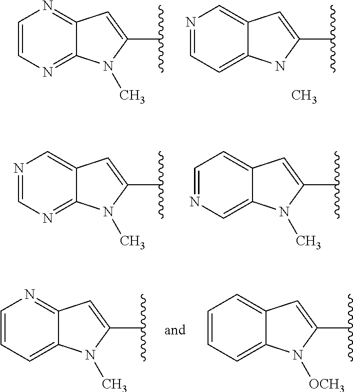

each of which can be optionally substituted as set forth above for the Compounds of Formula (Ic).

In an eighth aspect of this fifteenth embodiment, Z is selected from:

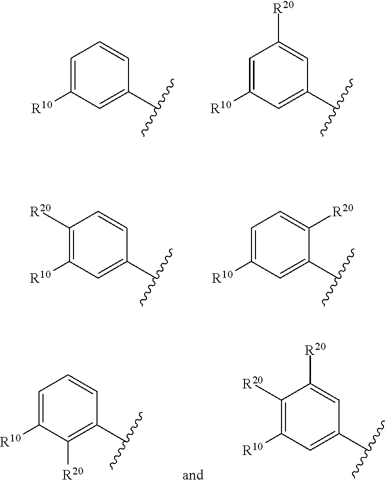

wherein each occurrence of R²⁰ is independently Cl, F, CN, —OCF₃ or —OCH₃; and R¹⁰ is selected from:

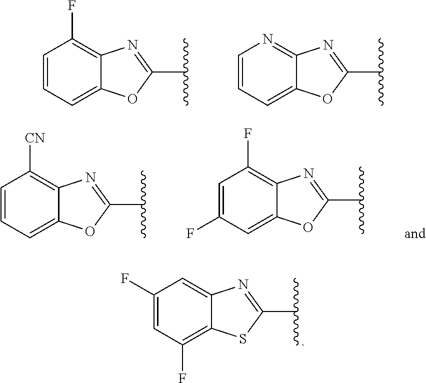

In a ninth aspect of this fifteenth embodiment, Z is selected from:

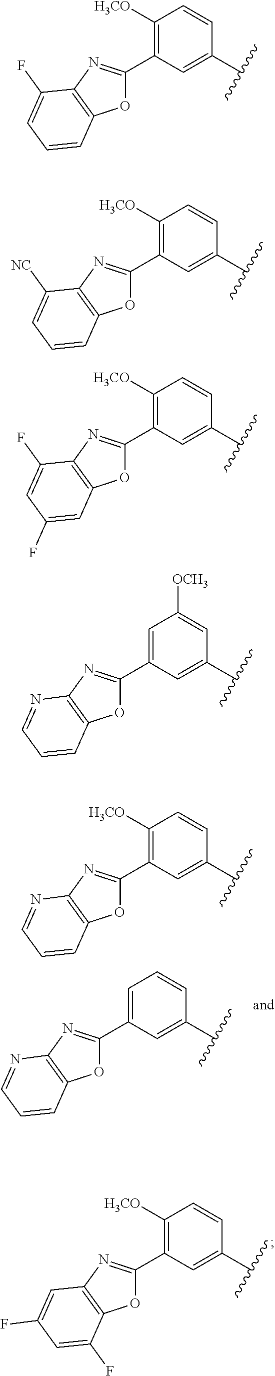

and R¹⁰ is selected from:

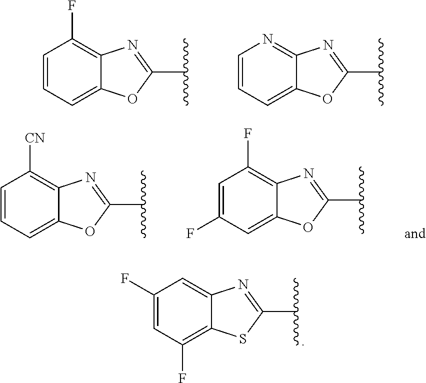

In a tenth aspect of this fifteenth embodiment, R³⁰ is —CH₃.

In an eleventh aspect of this fifteenth embodiment, R⁴⁰ is C₁-C₆ alkyl, C₁-C₆ haloalkyl, —(CH₂)ₜ—OH or —(CH₂)ₜ—CN, wherein t is an integer ranging from 0 to 6. In a first aspect of this fifth embodiment, R⁴⁰ is C₁-C₆ alkyl. In a second aspect of this fifth embodiment, R⁴⁰ is —CH₃, —(CH₂)₃—CN, —CH₂CH₂F, or —CH₂CH₂C(CH₃)₂—OH. In a third aspect of this fifth embodiment, R⁴⁰ is —CH₃.

In a twelfth aspect of this fifteenth embodiment, R⁵⁰ is C₁-C₆ alkyl. In a first aspect of this sixth embodiment, R⁵⁰ is C₆-C₁₀ aryl. In a second aspect of this sixth embodiment, R⁵⁰ is C₃-C₇ cycloalkyl. In a third aspect of this sixth embodiment, R⁵⁰ is —CH₃, phenyl or cyclopropyl. In a fourth aspect of this sixth embodiment, R⁵⁰ is —CH₃.

In a thirteenth aspect of this fifteenth embodiment, only one R⁶⁰ group is present. In a first aspect of this seventh embodiment, R⁶⁰ represents a single halo group. In a second aspect of this seventh embodiment, R⁶⁰ represents a single F group. In a third aspect of this seventh embodiment, R⁶⁰ represents a single F group at the para position of the phenyl ring to which it is attached.

In a fourteenth aspect of this fifteenth embodiment, R⁴⁰ is —CH₃, —(CH₂)₃—CN, —CH₂CH₂F or —CH₂CH₂C(CH₃)₂—OH, and R⁵⁰ is —CH₃. In a first aspect of this eighth embodiment, R⁴⁰ and R⁵⁰ are each —CH₃.

In a fifteenth aspect of this fifteenth embodiment, R³⁰, R⁴⁰ and R⁵⁰ are each —CH₃.

In a sixteenth aspect of this fifteenth embodiment, R⁴⁰ is —CH₃, —(CH₂)₃—CN, —CH₂CH₂F or —CH₂CH₂C(CH₃)₂—OH; R⁵⁰ is —CH₃; and R⁶⁰ represents a single F group at the para position of the phenyl ring to which it is attached.

In a seventeenth aspect of this fifteenth embodiment, R³⁰ is —CH₃; R⁴⁰ is —CH₃, —(CH₂)₃—CN, —CH₂CH₂F or —CH₂CH₂C(CH₃)₂—OH; R⁵⁰ is —CH₃; and R⁶⁰ represents a single F group at the para position of the phenyl ring to which it is attached. In a first aspect of this eleventh embodiment, R³⁰, R⁴⁰ and R⁵⁰ are each —CH₃ and R⁶⁰ represents a single F group at the para position of the phenyl ring to which it is attached.

In a sixteenth embodiment of the present invention, the Compounds of Formula (I) have the formula (Id):

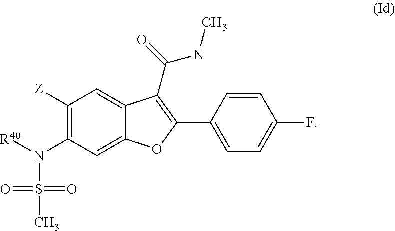

and pharmaceutically acceptable salts thereof,
wherein:

Z is:

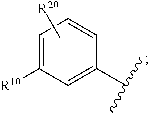

R¹⁰ is a 9-membered bicyclic heteroaryl group, wherein said 9-membered bicyclic heteroaryl group is optionally substituted with up to 2 groups, which can be the same or different, and are selected from halo, C₁-C₆ alkyl, —(CH₂)ₜ—N(R⁷⁰)₂, —(CH₂)ₜ—OH, —(CH₂)ₜ—O—(C₁-C₆ alkyl), —CF₃, —NHC(O)-heterocyclyl, —NHC(O)—(C₁-C₆ alkyl), —C(O)NH—(C₁-C₆ alkyl), —C(O)OH, —C(O)O—(C₁-C₆ alkyl), —NHC(O)-aryl, —NHSO₂-aryl, —NHSO₂-alkyl, —O—SO₂-alkyl, —O—(C₁-C₆ alkyl) and —CN, wherein the heterocyclyl moiety of said —NHC(O)-heterocyclyl group can be optionally substituted on a ring carbon or ring nitrogen atom with a —C(O)O—(C₁-C₆ alkyl) group;

R²⁰ represents up to 2 optional substituents, which can be the same or different, and are selected from halo, C₁-C₆ alkyl, —O—(C₁-C₆ alkyl), —O—(CH₂)ₜ—OH, —O—(CH₂)ₜ-heterocyclyl, —O—(C₁-C₆ haloalkyl), —O—SO₂—(C₁-C₆ alkyl) and —CN;

R⁴⁰ is C₁-C₆ alkyl, C₁-C₆ haloalkyl, —(CH₂)ₜ—OH or —(CH₂)ᵣ—CN; and

each occurrence oft is independently an integer ranging from 0 to 6.

In a first aspect of this sixteenth embodiment, R¹⁰ is selected from:

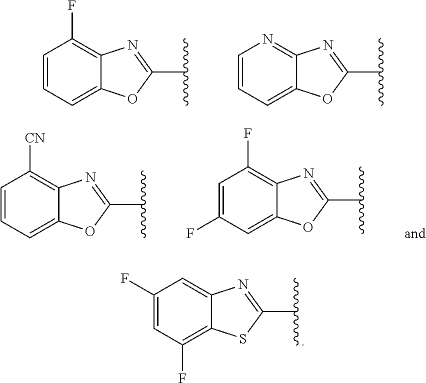

In a second aspect of this sixteenth embodiment, Z is selected from:

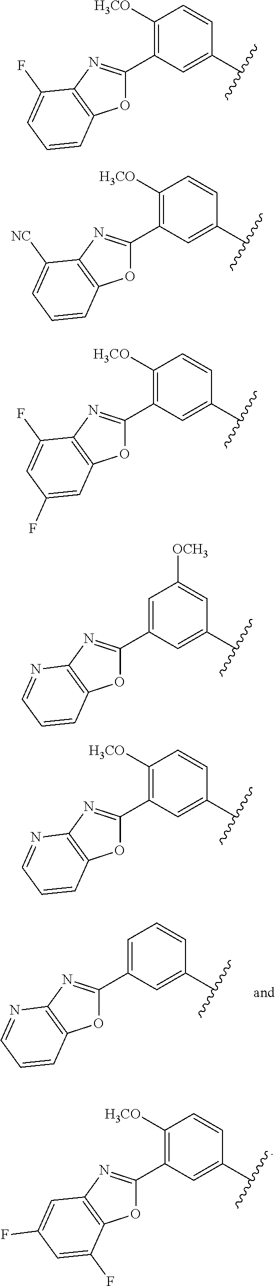

In a third aspect of this sixteenth embodiment, Z is:

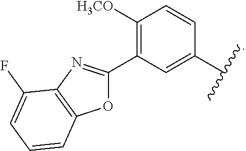

In a fourth aspect of this sixteenth embodiment, Z is:

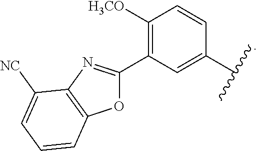

In a fifth aspect of this sixteenth embodiment, Z is:

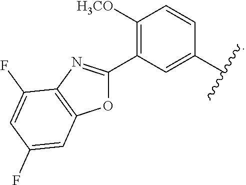

In a sixth aspect of this sixteenth embodiment, Z is:

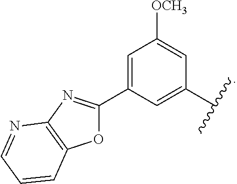

In a seventh aspect of this sixteenth embodiment, Z is:

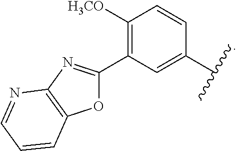

In a eighth aspect of this sixteenth embodiment, Z is:

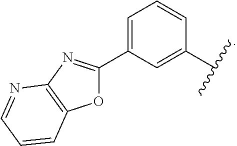

In an ninth aspect of this sixteenth embodiment, Z is:

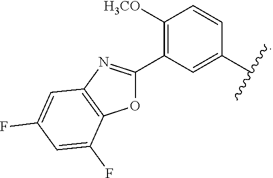

In a tenth aspect of this sixteenth embodiment, R⁴⁰ is C₁-C₆ alkyl. In an eleventh aspect of this sixteenth embodiment, R⁴⁰ is —CH₃, —(CH₂)₃—CN, —CH₂CH₂F, or —CH₂CH₂C(CH₃)₂—OH.

In a twelfth aspect of this sixteenth embodiment, Z is selected from:

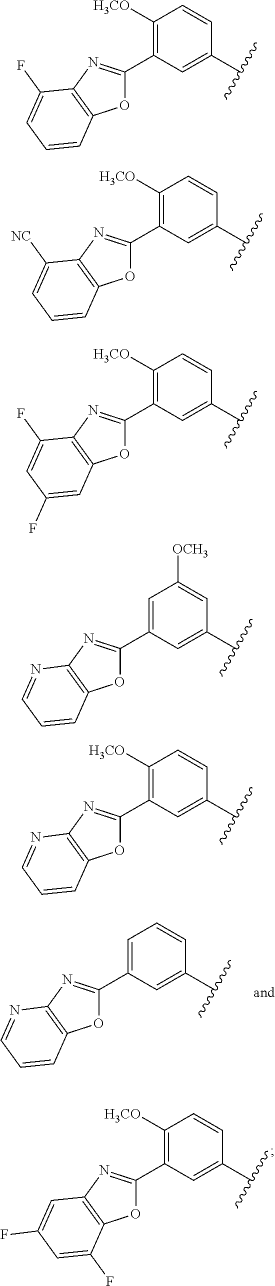

and

### R⁴⁰ is —CH₃, —(CH₂)₃—CN, —CH₂CH₂F, or —CH₂CH₂C(CH₃)₂—OH.

In a thirteenth aspect of this sixteenth embodiment, Z is selected from:

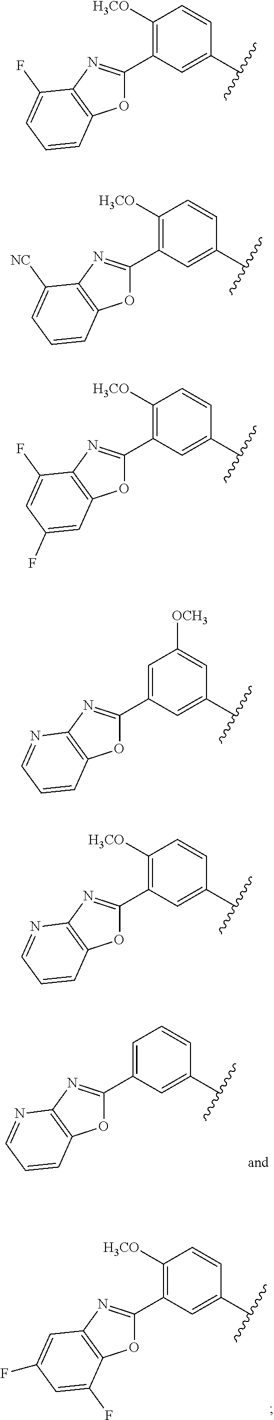

and

### R⁴⁰ is —CH₃.

In a fourteenth aspect of this sixteenth embodiment, Z is:

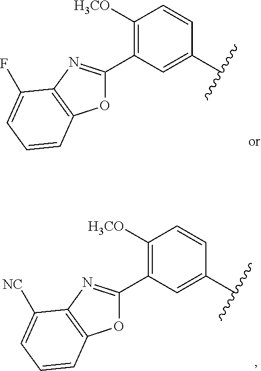

and

### R⁴⁰ is —CH₃.

In a seventeenth embodiment of the present invention, the Compounds of Formula (I) have the formula (Ie):

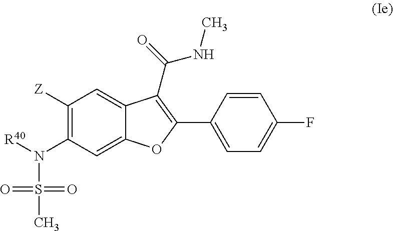

and pharmaceutically acceptable salts thereof,
wherein:

Z is a 5- or 6-membered heteroaryl group, which is substituted with one R¹⁰ group and optionally substituted with up to two R²⁰ groups;

R¹⁰ is a 9-membered bicyclic heteroaryl group, wherein said 9-membered bicyclic heteroaryl group is optionally substituted with up to 2 groups, which can be the same or different, and are selected from halo, C₁-C₆ alkyl, —(CH₂)ₜ—N(R⁷⁰)₂, —(CH₂)ₜ—OH, —(CH₂)ₜ—O—(C₁-C₆ alkyl), —CF₃, —NHC(O)-heterocyclyl, —NHC(O)—(C₁-C₆ alkyl), —C(O)NH—(C₁-C₆ alkyl), —C(O)OH, —C(O)O—(C₁-C₆ alkyl), —NHC(O)-aryl, —NHSO₂-aryl, —NHSO₂-alkyl, —O—SO₂-alkyl, —O—(C₁-C₆ alkyl) and —CN, wherein the heterocyclyl moiety of said —NHC(O)-heterocyclyl group can be optionally substituted on a ring carbon or ring nitrogen atom with a —C(O)O—(C₁-C₆ alkyl) group;

R²⁰ represents up to 2 optional substituents, which can be the same or different, and are selected from halo, C₁-C₆ alkyl, —O—(C₁-C₆ alkyl) and —CN;

R⁴⁰ is C₁-C₆ alkyl; and

each occurrence of t is independently an integer ranging from 0 to 6.

In a first aspect of this seventeenth embodiment, Z is pyridyl or thiophenyl.

In a second aspect of this seventeenth embodiment, Z is pyridyl, which is optionally substituted with up to 2 groups, each independently selected from methoxy, fluoro or —CN.

In a third aspect of this seventeenth embodiment, R¹⁰ is selected from:

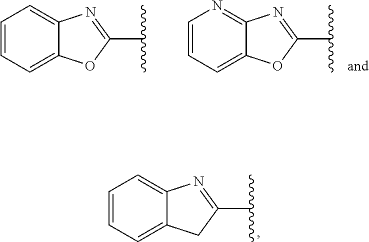

each of which can be optionally substituted as set forth above in formula (Ie).

In a fourth aspect of this seventeenth embodiment, R¹⁰ is selected from:

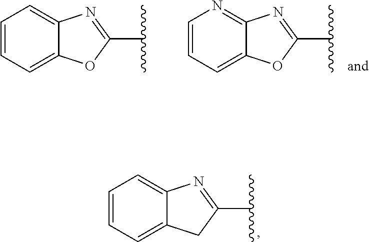

each of which can be optionally substituted with 1 or 2 groups, independently selected from halo, —CN and —O(C₁-C₆ alkyl).

In a fifth aspect of this seventeenth embodiment, R¹⁰ is selected from:

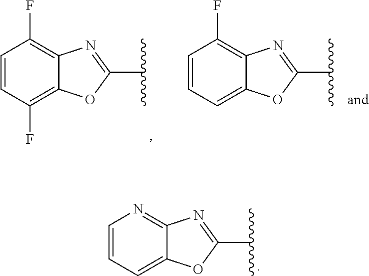

In a fifth aspect of this seventeenth embodiment, Z is;

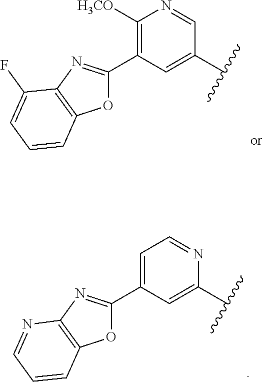

In a sixth aspect of this seventeenth embodiment, Z is:

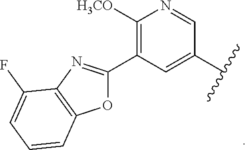

In a seventh aspect of this seventeenth embodiment, Z is:

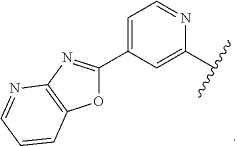

In a tenth aspect of this seventeenth embodiment, R⁴⁰ is methyl.

In an eleventh aspect of this seventeenth embodiment, Z is:

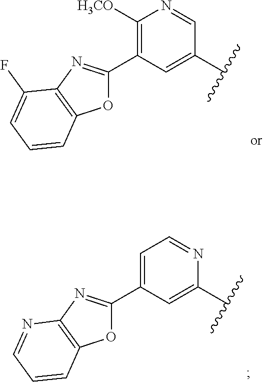

and

### R⁴⁰ is —CH₃.

In an eighteenth embodiment of the invention, for the compounds of formula (I), variables R¹, R², R³, R⁴ and n are selected independently of each other.

In a nineteenth embodiment of the invention, the compounds of formula (I) are in isolated and purified form.

In another embodiment of the invention, the compound of the invention is selected from the exemplary species depicted in Examples 1-880 as shown below, and pharmaceutically acceptable salts thereof.

Other embodiments of the present invention include the following:

(a) A pharmaceutical composition comprising an effective amount of a compound of formula (I) and a pharmaceutically acceptable carrier.

(b) The pharmaceutical composition of (a), further comprising a second therapeutic agent selected from the group consisting of HCV antiviral agents, immunomodulators, and anti-infective agents.

(c) The pharmaceutical composition of (b), wherein the HCV antiviral agent is an antiviral selected from the group consisting of direct inhibitors of HCV, including but not limited to NS3 and NS3/4A protease inhibitors, NS5A inhibitors and HCV NS5B polymerase inhibitors.

(d) A pharmaceutical combination that is (i) a compound of formula (I) and (ii) a second therapeutic agent selected from the group consisting of HCV antiviral agents, immunomodulators, and anti-infective agents; wherein the compound of formula (I) and the second therapeutic agent are each employed in an amount that renders the combination effective for inhibiting HCV NS5B activity, or for inhibiting HCV viral replication, or for treating HCV infection and/or reducing the likelihood or severity of symptoms of HCV infection.

(e) The combination of (d), wherein the HCV antiviral agents are one or more antiviral agents selected from the group consisting of direct inhibitors of HCV, including but not limited to NS3 and NS3/4A protease inhibitors, NS5A inhibitors and HCV NS5B polymerase inhibitors.

(f) A use of a compound of formula (I) in the preparation of a medicament for inhibiting HCV NS5B activity in a subject in need thereof.

(g) A use of a compound of formula (I) in the preparation of a medicament for preventing and/or treating infection by HCV in a subject in need thereof.

(h) A method of treating HCV infection and/or reducing the likelihood or severity of symptoms of HCV infection in a subject in need thereof, which comprises administering to the subject an effective amount of a compound of formula (I).

(i) The method of (h), wherein the compound of formula (I) is administered in combination with an effective amount of at least one second therapeutic agent selected from the group consisting of HCV antiviral agents, immunomodulators, and anti-infective agents.

(j) The method of (i), wherein the HCV antiviral agent is an antiviral selected from the group consisting of direct inhibitors of HCV, including but not limited to NS3 and NS3/4A protease inhibitors, NS5A inhibitors and HCV NS5B polymerase inhibitors.

(k) A method of inhibiting HCV viral replication and/or HCV viral production in a cell-based system, which comprises administering to the subject an effective amount of a compound of formula (I) in combination with an effective amount of at least one second therapeutic agent selected from the group consisting of HCV antiviral agents, immunomodulators, and anti-infective agents.

(l) The method of (k), wherein the HCV antiviral agent is an antiviral selected from the group consisting of direct inhibitors of HCV, including but not limited to NS3 and NS3/4A protease inhibitors, NS5A inhibitors and HCV NS5B polymerase inhibitors.

(m) A method of inhibiting HCV NS5B activity in a subject in need thereof, which comprises administering to the subject the pharmaceutical composition of (a), (b), or (c) or the combination of (d) or (e).

(n) A method of treating HCV infection and/or reducing the likelihood or severity of symptoms of HCV infection in a subject in need thereof, which comprises administering to the subject the pharmaceutical composition of (a), (b), or (c) or the combination of (d) or (e).

In the embodiments of the compounds and salts provided above, it is to be understood that each embodiment may be combined with one or more other embodiments, to the extent that such a combination provides a stable compound or salt and is consistent with the description of the embodiments. It is further to be understood that the embodiments of compositions and methods provided as (a) through (n) above are understood to include all embodiments of the compounds and/or salts, including such embodiments as result from combinations of embodiments.

Additional embodiments of the invention include the pharmaceutical compositions, combinations, uses and methods set forth in (a) through (n) above, wherein the compound of the present invention employed therein is a compound of one of the embodiments, aspects, classes, sub-classes, or features of the compounds described above. In all of these embodiments, the compound may optionally be used in the form of a pharmaceutically acceptable salt or hydrate as appropriate.

The present invention also includes a compound of the present invention for use (i) in, (ii) as a medicament for, or (iii) in the preparation of a medicament for: (a) inhibiting HCV NS5B activity, or (b) inhibiting HCV viral replication, or (c) treating HCV infection and/or reducing the likelihood or severity of symptoms of HCV infection, or (d) use in medicine. In these uses, the compounds of the present invention can optionally be employed in combination with one or more second therapeutic agents selected from HCV antiviral agents, anti-infective agents, and immunomodulators.

As used herein, all ranges are inclusive, and all sub-ranges are included within such ranges, although not necessarily explicitly set forth. In addition, the term “or,” as used herein, denotes alternatives that may, where appropriate, be combined; that is, the term “or” includes each listed alternative separately as well as their combination.

As used herein, the term “alkyl” refers to any linear or branched chain alkyl group having a number of carbon atoms in the specified range. Thus, for example, “C₁₋₆ alkyl” (or “C₁-C₆ alkyl”) refers to all of the hexyl alkyl and pentyl alkyl isomers as well as n-, iso-, sec- and t-butyl, n- and isopropyl, ethyl and methyl. As another example, “C₁₋₄ alkyl” refers to n-, iso-, sec- and t-butyl, n- and isopropyl, ethyl and methyl. Alkyl groups may be substituted as indicated.

The term “halogenated” refers to a group or molecule in which a hydrogen atom has been replaced by a halogen. Similarly, the term “haloalkyl” refers to a halogenated alkyl group. The term “halogen” (or “halo”) refers to atoms of fluorine, chlorine, bromine and iodine (alternatively referred to as fluoro, chloro, bromo, and iodo).

The term “alkoxy” refers to an “alkyl-O—” group. Alkoxy groups may be substituted as indicated.

The term “cycloalkyl” refers to any cyclic ring of an alkane or alkene having a number of carbon atoms in the specified range. Thus, for example, “C₃₋₈ cycloalkyl” (or “C₃-C₈ cycloalkyl”) refers to cyclopropyl, cyclobutyl, cyclopentyl, cyclopentenyl, cyclohexyl, cyclohexenyl, cycloheptyl, cycloheptenyl, cyclooctyl, and cyclooctenyl. The term “cycloalkoxy” refers to a “cycloalkyl-O—” group. Cycloalkyl groups may be substituted as indicated.

The term “aryl” (or “aryl ring system”) refers to aromatic mono- and poly-carbocyclic ring systems wherein the individual carbocyclic rings in the polyring systems are fused or attached to each other via a single bond. As used herein, the term aryl includes aromatic mono- and poly-carbocyclic ring systems that include from 0 to 4 heteroatoms (non-carbon atoms) that are independently chosen from N, O and S. Suitable aryl groups include phenyl, naphthyl, biphenylenyl, pyridinyl, pyrimidinyl and pyrrolyl, as well as those discussed below. Aryl groups may be substituted as indicated. Aryl ring systems may include, where appropriate, an indication of the variable to which a particular ring atom is attached. Unless otherwise indicated, substituents to the aryl ring systems can be attached to any ring atom, provided that such attachment results in formation of a stable ring system.

The term “carbocycle” (and variations thereof such as “carbocyclic”) as used herein, unless otherwise indicated, refers to (i) a C₅ to C₇ monocyclic, saturated or unsaturated ring, or (ii) a, C₈ to C₁₀ bicyclic saturated or unsaturated ring system. Each ring in (ii) is either independent of, or fused to, the other ring, and each ring is saturated or unsaturated. Carbocycle groups may be substituted as indicated. When the carbocycles contain one or more heteroatoms independently chosen from N, O and S, the carbocycles may also be referred to as “heterocycles,” as defined below. The carbocycle may be attached to the rest of the molecule at any carbon or nitrogen atom that results in a stable compound. The fused bicyclic carbocycles are a subset of the carbocycles; i.e., the term “fused bicyclic carbocycle” generally refers to a C₈ to C₁₀ bicyclic ring system in which each ring is saturated or unsaturated and two adjacent carbon atoms are shared by each of the rings in the ring system. A fused bicyclic carbocycle in which both rings are saturated is a saturated bicyclic ring system. Saturated carbocyclic rings are also referred to as cycloalkyl rings, e.g., cyclopropyl, cyclobutyl, etc. A fused bicyclic carbocycle in which one or both rings are unsaturated is an unsaturated bicyclic ring system. Carbocycle ring systems may include, where appropriate, an indication of the variable to which a particular ring atom is attached. Unless otherwise indicated, substituents to the ring systems can be attached to any ring atom, provided that such attachment results in formation of a stable ring system.

Unless indicated otherwise, the term “heterocycle” (and variations thereof such as “heterocyclic” or “heterocyclyl”) broadly refers to (i) a stable 5- to 7-membered, saturated or unsaturated monocyclic ring, or (ii) a stable 8- to 10-membered bicyclic ring system, wherein each ring in (ii) is independent of, or fused to, the other ring or rings and each ring is saturated or unsaturated, and the monocyclic ring or bicyclic ring system contains one or more heteroatoms (e.g., from 1 to 6 heteroatoms, or from 1 to 4 heteroatoms) independently selected from N, O and S and a balance of carbon atoms (the monocyclic ring typically contains at least one carbon atom and the bicyclic ring systems typically contain at least two carbon atoms); and wherein any one or more of the nitrogen and sulfur heteroatoms is optionally oxidized, and any one or more of the nitrogen heteroatoms is optionally quaternized. Unless otherwise specified, the heterocyclic ring may be attached at any heteroatom or carbon atom, provided that attachment results in the creation of a stable structure. Heterocycle groups may be substituted as indicated, and unless otherwise specified, the substituents may be attached to any atom in the ring, whether a heteroatom or a carbon atom, provided that a stable chemical structure results. Representative examples include piperidinyl, piperazinyl, azepanyl, pyrrolidinyl, pyrazolidinyl, imidazolidinyl, oxazolidinyl, isoxazolidinyl, morpholinyl, thiomorpholinyl, thiazolidinyl, isothiazolidinyl, and tetrahydrofuryl (or tetrahydrofuranyl). Unless expressly stated to the contrary, the term “heteroaryl ring system” refers to aryl ring systems, as defined above, that include from 1 to 4 heteroatoms (non-carbon atoms) that are independently chosen from N, O and S. In the case of substituted heteraromatic rings containing at least one nitrogen atom (e.g., pyridine), such substitutions can be those resulting in N-oxide formation. Representative examples of heteroaromatic rings include pyridyl, pyrrolyl, pyrazinyl, pyrimidinyl, pyridazinyl, thienyl (or thiophenyl), thiazolyl, furanyl, imidazolyl, pyrazolyl, triazolyl, tetrazolyl, oxazolyl, isooxazolyl, oxadiazolyl, thiazolyl, isothiazolyl, and thiadiazolyl. Representative examples of bicyclic heterocycles include benzotriazolyl, indolyl, isoindolyl, indazolyl, indolinyl, isoindolinyl, quinoxalinyl, quinazolinyl, cinnolinyl, chromanyl, isochromanyl, tetrahydroquinolinyl, quinolinyl, tetrahydroisoquinolinyl, isoquinolinyl, 2,3-dihydrobenzofuranyl, 2,3-dihydrobenzo-1,4-dioxinyl and benzo-1,3-dioxolyl.

Unless otherwise specifically noted as only “substituted”, alkyl, cycloalkyl, and aryl groups are not substituted. Preferably, the substituents are selected from the group which includes, but is not limited to, halo, C₁-C₂₀ alkyl, —CF₃, —NH₂, —N(C₁-C₆ alkyl)₂, —NO₂, oxo, —CN, —N₃, —OH, —O(C₁-C₆ alkyl), C₃-C₁₀ cycloalkyl, C₂-C₆ alkenyl, C₂-C₆ alkynyl, (C₀-C₆ alkyl) S(O)₀₋₂—, aryl-S(O)₀₋₂—, (C₀-C₆ alkyl)S(O)₀₋₂(C₀-C₆ alkyl)-, (C₀-C₆ alkyl)C(O)NH—, H₂N—C(NH)—, —O(C₁-C₆ alkyl)CF₃, (C₀-C₆ alkyl)C(O)—, (C₀-C₆ alkyl)OC(O)—, (C₀-C₆alkyl)O(C₁-C₆ alkyl)-, (C₀-C₆ alkyl)C(O)₁₋₂(C₀-C₆ alkyl)-, (C₀-C₆ alkyl)OC(O)NH—, aryl, aralkyl, heteroaryl, heterocyclylalkyl, halo-aryl, halo-aralkyl, halo-heterocycle and halo-heterocyclylalkyl.

As used herein, the term “compound” is intended to encompass chemical agents described by generic formula (I) in all forms, including hydrates and solvates of such chemical agents.

In the compounds of formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of formula (I). For example, different isotopic forms of hydrogen (H) include protium ('H) and deuterium (²H or D). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within formula (I) can be prepared without undue experimentation by conventional techniques well known to those skilled in the art or by processes analogous to those described in the Schemes and Examples herein using appropriate isotopically-enriched reagents and/or intermediates.

Unless expressly stated to the contrary, all ranges cited herein are inclusive. For example, a heteroaryl ring described as containing from “0 to 3 heteroatoms” means the ring can contain 0, 1, 2, or 3 heteroatoms. It is also to be understood that any range cited herein includes within its scope all of the sub-ranges within that range. The oxidized forms of the heteroatoms N and S are also included within the scope of the present invention.

When any variable (for example, R¹ or R³) occurs more than one time in any constituent or in formula (I) or in any other formula depicting and describing compounds of the invention, its definition on each occurrence is independent of its definition at every other occurrence. Also, combinations of substituents and/or variables are permissible only if such combinations result in stable compounds.

Unless expressly stated to the contrary, substitution by a named substituent is permitted on any atom provided such substitution is chemically allowed and results in a stable compound. A “stable” compound is a compound that can be prepared and isolated and whose structure and properties remain or can be caused to remain essentially unchanged for a period of time sufficient to allow use of the compound for the purposes described herein (e.g., therapeutic or prophylactic administration to a subject).

As a result of the selection of substituents and substituent patterns, certain of the compounds of the present invention can have asymmetric centers and can occur as mixtures of stereoisomers, or as individual diastereomers, or enantiomers. All isomeric forms of these compounds, whether isolated or in mixtures, are within the scope of the present invention.

As would be recognized by one of ordinary skill in the art, certain of the compounds of the present invention can exist as tautomers. For the purposes of the present invention a reference to a compound of formula (I) is a reference to the compound per se, or to any one of its tautomers per se, or to mixtures of two or more tautomers.

The compounds of the present inventions are useful in the inhibition of HCV replication (e.g., HCV NS5B activity), the treatment of HCV infection and/or reduction of the likelihood or severity of symptoms of HCV infection. For example, the compounds of this invention are useful in treating infection by HCV after suspected past exposure to HCV by such means as blood transfusion, exchange of body fluids, bites, accidental needle stick, or exposure to patient blood during surgery.

The compounds of this invention are useful in the preparation and execution of screening assays for antiviral compounds. For example, the compounds of this invention are useful for identifying resistant HCV replicon cell lines harboring mutations within NS5B, which are excellent screening tools for more powerful antiviral compounds. Furthermore, the compounds of this invention are useful in establishing or determining the binding site of other antivirals to the HCV replicase.

The compounds of the present invention may be administered in the form of pharmaceutically acceptable salts. The term “pharmaceutically acceptable salt” refers to a salt that possesses the effectiveness of the parent compound and that is not biologically or otherwise undesirable (e.g., is neither toxic nor otherwise deleterious to the recipient thereof). Suitable salts include acid addition salts that may, for example, be formed by mixing a solution of the compound of the present invention with a solution of a pharmaceutically acceptable acid such as hydrochloric acid, sulfuric acid, acetic acid, trifluoroacetic acid, or benzoic acid. Many of the compounds of the invention carry an acidic moiety, in which case suitable pharmaceutically acceptable salts thereof can include alkali metal salts (e.g., sodium or potassium salts), alkaline earth metal salts (e.g., calcium or magnesium salts), and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

The term “administration” and variants thereof (e.g., “administering” a compound) in reference to a compound of the invention mean providing the compound or a prodrug of the compound to the individual in need of treatment. When a compound of the invention is provided in combination with one or more other active agents (e.g., antiviral agents useful for treating HCV infection), “administration” and its variants are each understood to include concurrent and sequential provision of the compound or salt and other agents.

As used herein, the term “composition” is intended to encompass a product comprising the specified ingredients, as well as any product which results, directly or indirectly, from combining the specified ingredients.

By “pharmaceutically acceptable” is meant that the ingredients of the pharmaceutical composition must be compatible with each other and not deleterious to the recipient thereof.

The term “subject” (alternatively referred to herein as “patient”), as used herein, refers to an animal, preferably a mammal, most preferably a human, who has been the object of treatment, observation or experiment.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of one or more symptoms of the disease or condition being treated. In another embodiment, the effective amount is a “prophylactically effective amount” for reduction of the severity or likelihood of one or more symptoms of the disease or condition. In another embodiment, the effective amount is a “therapeutically effective amount” for inhibition of HCV viral replication and/or HCV viral production. The term also includes herein the amount of active compound sufficient to inhibit HCV NS5B activity and thereby elicit the response being sought (i.e., an “inhibition effective amount”). When the active compound (i.e., active ingredient) is administered as the salt, references to the amount of active ingredient are to the free acid or free base form of the compound.

For the purposes of inhibiting HCV NS5B polymerase, treating HCV infection and/or reducing the likelihood or severity of symptoms of HCV infection and inhibiting HCV viral replication and/or HCV viral production, the compounds of the present invention, optionally in the form of a salt, can be administered by any means that produces contact of the active agent with the agent's site of action. They can be administered by one or more conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered by one or more of the following: orally, parenterally (including subcutaneous injections, intravenous, intramuscular, intrasternal injection or infusion techniques), by inhalation (such as in a spray form), or rectally, in the form of a unit dosage of a pharmaceutical composition containing an effective amount of the compound and conventional non-toxic pharmaceutically-acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as solubility aids. Injectable solutions can be prepared according to methods known in the art wherein the carrier comprises a saline solution, a glucose solution or a solution containing a mixture of saline and glucose. Further description of methods suitable for use in preparing pharmaceutical compositions of the present invention and of ingredients suitable for use in said compositions is provided in Remington's Pharmaceutical Sciences, 18^{th} edition (ed. A. R. Gennaro, Mack Publishing Co., 1990).

The compounds of this invention can be administered orally in a dosage range of 0.001 to 1000 mg/kg of mammal (e.g., human) body weight per day in a single dose or in divided doses. One dosage range is 0.01 to 500 mg/kg body weight per day orally in a single dose or in divided doses. Another dosage range is 0.1 to 100 mg/kg body weight per day orally in single or divided doses. For oral administration, the compositions can be provided in the form of tablets or capsules containing 1.0 to 500 mg of the active ingredient, particularly 1, 5, 10, 15, 20, 25, 50, 75, 100, 150, 200, 250, 300, 400, and 500 mg of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, HCV viral genotype, viral resistance, and the host undergoing therapy.

As noted above, the present invention also relates to a method of inhibiting HCV NS5B activity, inhibiting HCV viral replication and/or HCV viral production, treating HCV infection and/or reducing the likelihood or severity of symptoms of HCV infection with a compound of the present invention in combination with one or more therapeutic agents and a pharmaceutical composition comprising a compound of the present invention and one or more therapeutic agents selected from the group consisting of a HCV antiviral agent, an immunomodulator, and an anti-infective agent. Such therapeutic agents active against HCV include, but are not limited to, ribavirin, levovirin, viramidine, thymosin alpha-1, R7025 (an enhanced interferon (Roche)), interferon-β, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. The combination of pegylated-interferon and ribaviron represents the current Standard of Care for HCV treatment. The combination of one or more compounds of the present invention with the Standard of Care for HCV treatment, pegylated-interferon and ribaviron is specifically contemplated as being encompassed by the present invention. Interferon-α includes, but is not limited to, recombinant interferon-α2a (such as ROFERON interferon available from Hoffmann-LaRoche, Nutley, N.J.), pegylated interferon-α2a (PEGASUS), interferon-α1b (such as INTRON-A interferon available from Schering Corp., Kenilworth, N.J.), pegylated interferon-α2b (PEGINTRON), a recombinant consensus interferon (such as interferon alphacon-1), albuferon (interferon-α bound to human serum albumin (Human Genome Sciences)), and a purified interferon-α product. Amgen's recombinant consensus interferon has the brand name INFERGEN. Levovirin is the L-enantiomer of ribavirin which has shown immunomodulatory activity similar to ribavirin. Viramidine represents an analog of ribavirin disclosed in International Patent Application Publication WO 01/60379. In accordance with the method of the present invention, the individual components of the combination can be administered separately at different times during the course of therapy or concurrently in divided or single combination forms.

For the treatment of HCV infection, the compounds of the invention may also be administered in combination with an antiviral agent NS5B polymerase inhibitor, e.g., R7128 (Roche), valopicitabine (NM-283; Idenix) and 2′-F-2′-beta-methylcytidine (see also WO 2005/003147).

The compounds of the present invention also may be combined for the treatment of HCV infection with antiviral 2′-C-branched ribonucleosides disclosed in Rogers E. Harry-O'Kuru et al., A Short, Flexible Route toward 2′-C-Branched Ribonucleosides, 62 J. ORG. CHEM. 1754-59 (1997); Michael S. Wolfe & Rogers E. Harry-O'Kuru, A Concise 2′-C-Methylribonucleosides, 36(42) TETRAHEDRON LETTERS 7611-14 (1995); U.S. Pat. No. 3,480,613; and International Patent Application Publications WO 01/90121, WO 01/92282, WO 02/32920, WO 04/002999, WO 04/003000 and WO 04/002422; the entire contents of each of which are incorporated by reference. Such 2′-C-branched ribonucleosides include, but are not limited to, 2′-C-methyl-cytidine, 2′-C-methyl-uridine, 2′-C-methyl-adenosine, 2′-C-methyl-guanosine, and 9-(2-C-methyl-β-D-ribofuranosyl)-2,6-diaminopurine, and the corresponding amino acid ester of the ribose C-2′, C-3′, and C-5′ hydroxyls and the corresponding optionally substituted cyclic 1,3-propanediol esters of the 5′-phosphate derivatives.

For the treatment of HCV infection, the compounds of the present invention may also be administered in combination with an agent that is an inhibitor of HCV NS3 serine protease. HCV NS3 serine protease is an essential viral enzyme and has been described to be an excellent target for inhibition of HCV replication. Exemplary substrate and non-substrate based inhibitors of HCV NS3 protease inhibitors are disclosed in International Patent Application Publications WO 98/22496, WO 98/46630, WO 99/07733, WO 99/07734, WO 99/38888, WO 99/50230, WO 99/64442, WO 00/09543, WO 00/59929, WO 02/48116, WO 02/48172, WO 2008/057208 and WO 2008/057209, in British Patent No. GB 2 337 262, and in U.S. Pat. Nos. 6,323,180 and 7,470,664.

Further examples of HCV protease inhibitors useful in the present compositions and methods include, but are not limited to, the following compounds:

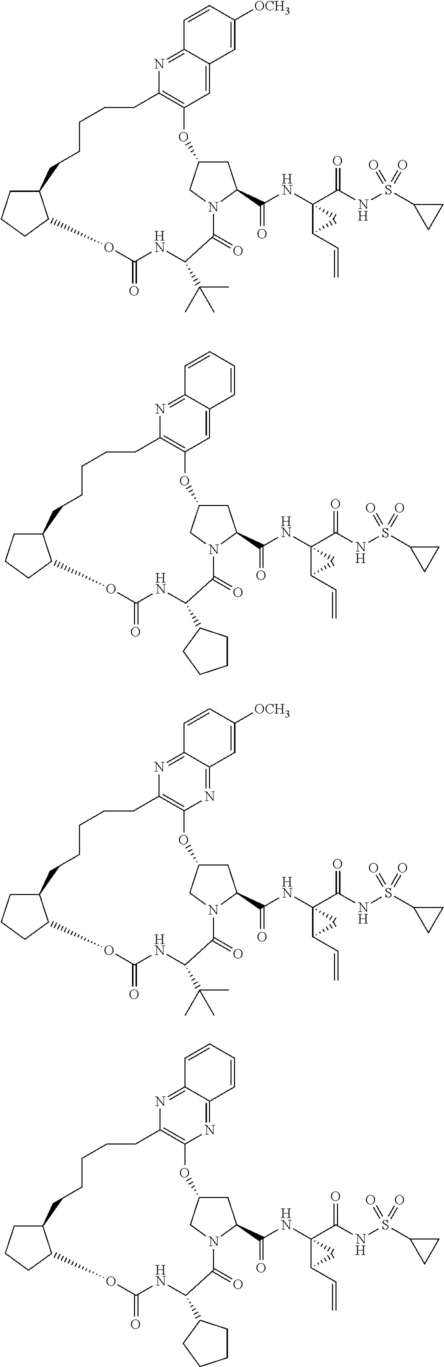

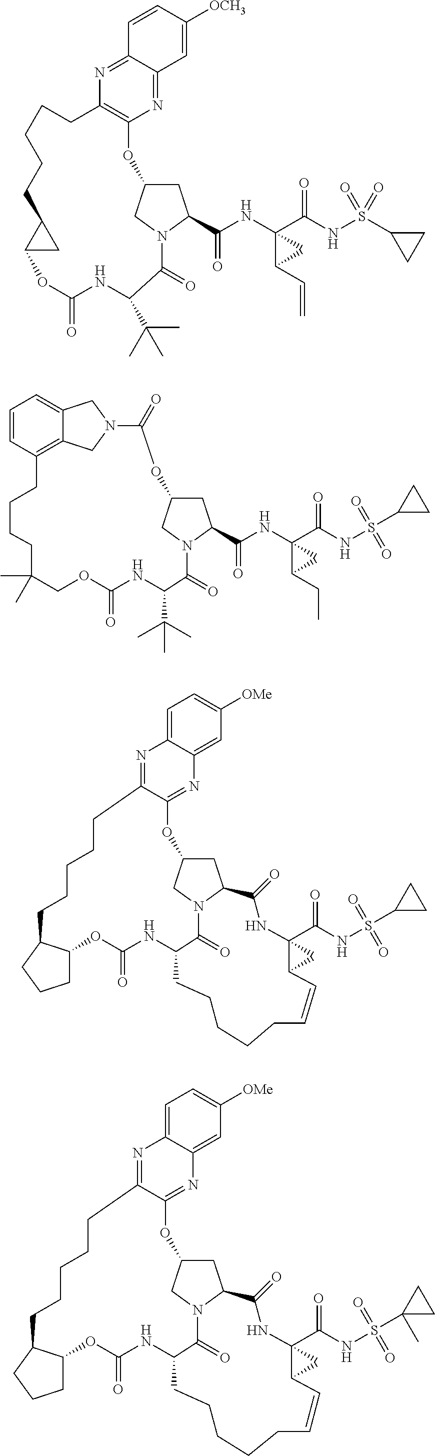

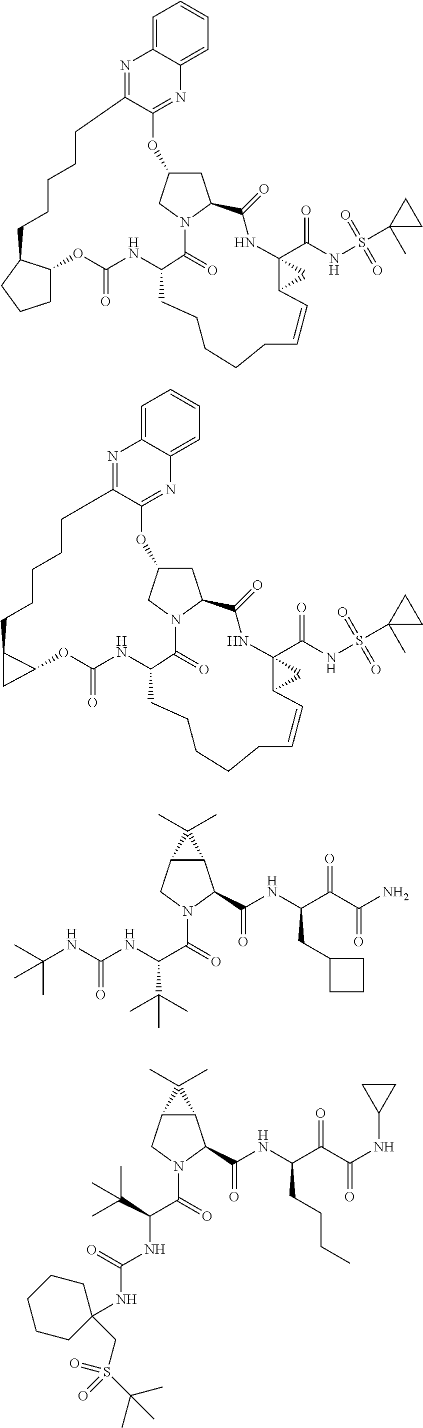

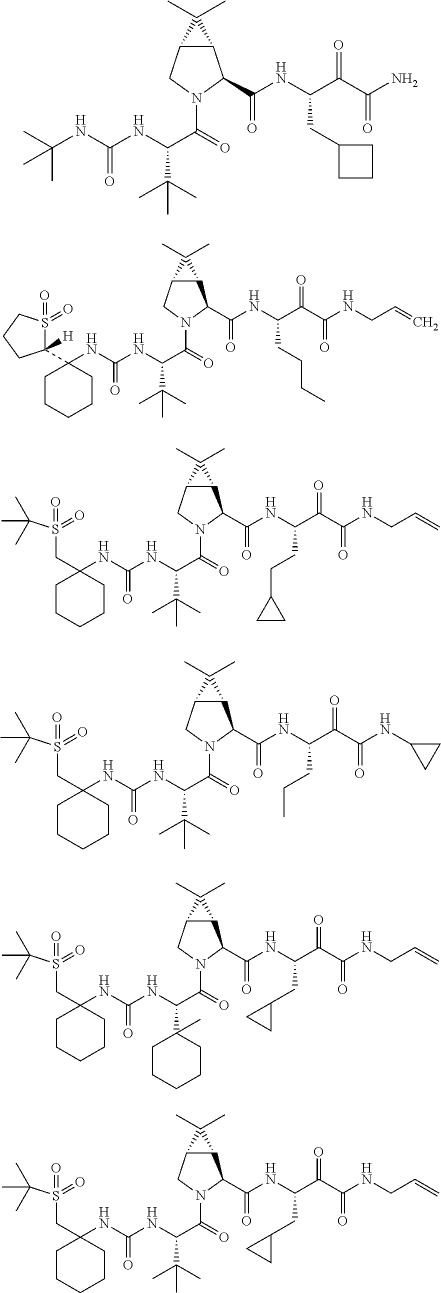

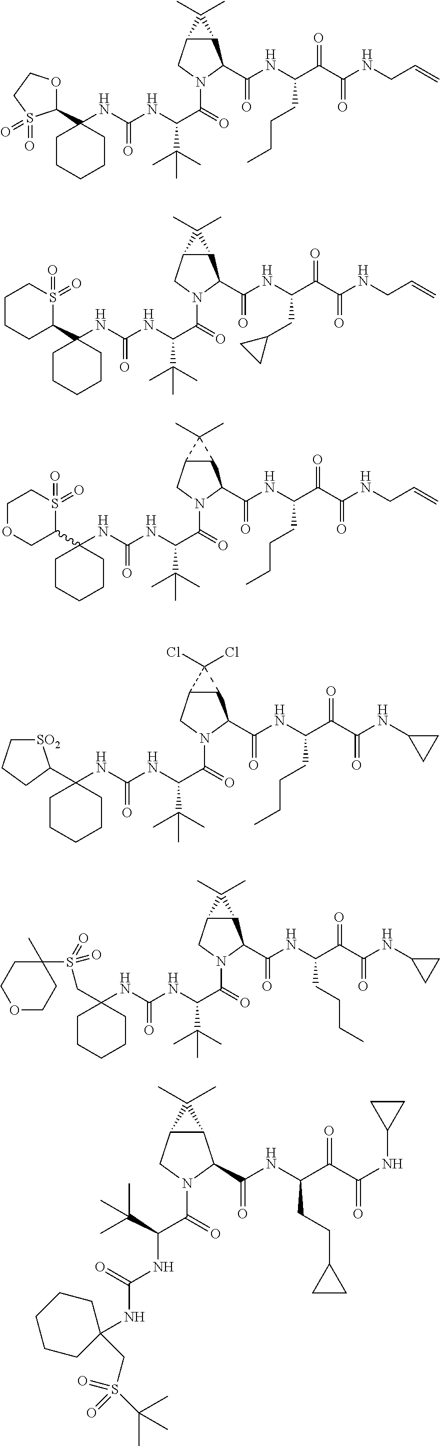

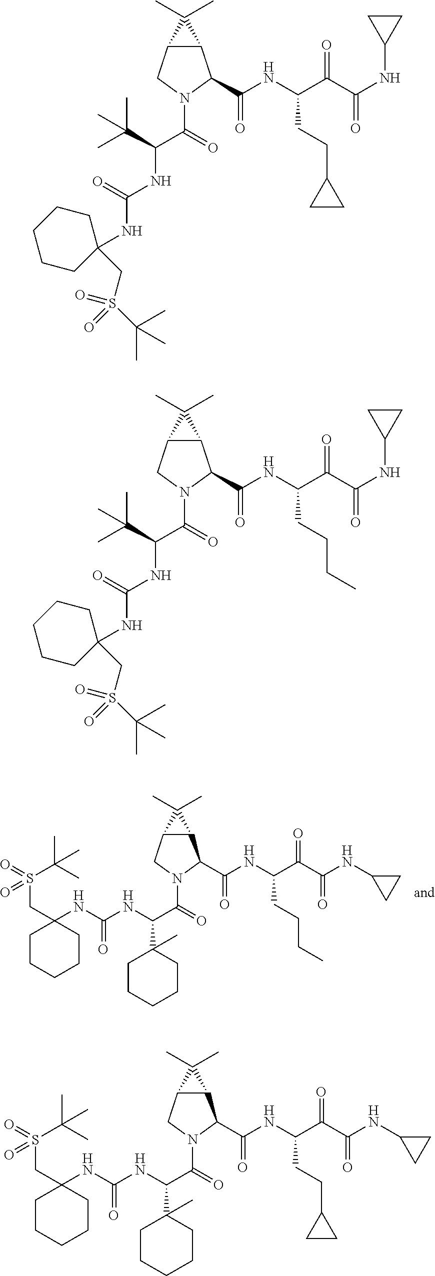

and pharmaceutically acceptable salts thereof.

The compounds of the present invention may also be combined for the treatment of HCV infection with nucleosides having anti-HCV properties, such as those disclosed in International Patent Application Publications WO 02/51425, WO 01/79246, WO 02/32920, WO 02/48165 and WO 2005/003147 (including R1656, (2′R)-2′-deoxy-2′-fluoro-2′-C-methylcytidine, shown as compounds 3-6 on page 77); WO 01/68663; WO 99/43691; WO 02/18404 and WO 2006/021341, and U.S. Patent Application Publication US 2005/0038240, including 4′-azido nucleosides such as R1626, 4′-azidocytidine; U.S. Patent Application Publications US 2002/0019363, US 2003/0236216, US 2004/0006007, US 2004/0063658 and US 2004/0110717; U.S. Pat. Nos. 7,105,499, 7,125,855, 7,202,224; and International Patent Application Publications WO 02/100415, WO 03/026589, WO 03/026675, WO 03/093290, WO 04/011478, WO 04/013300 and WO 04/028481; the content of each is incorporated herein by reference in its entirety.

For the treatment of HCV infection, the compounds of the present invention may also be administered in combination with an agent that is an inhibitor of HCV NS5B polymerase. Such HCV NS5B polymerase inhibitors that may be used as combination therapy include, but are not limited to, those disclosed in International Patent Application Publications WO 02/057287, WO 02/057425, WO 03/068244, WO 2004/000858, WO 04/003138 and WO 2004/007512; U.S. Pat. Nos. 6,777,392, 7,105,499, 7,125,855, 7,202,224 and U.S. Patent Application Publications US 2004/0067901 and US 2004/0110717; the content of each is incorporated herein by reference in its entirety.

In one embodiment, additional nucleoside HCV NS5B polymerase inhibitors that are used in combination with the present HCV NS5B inhibitors are selected from the following compounds: 4-amino-7-(2-C-methyl-β-D-arabinofuranosyl)-7H-pyrrolo[2,3-d]pyrimidine; 4-amino-7-(2-C-methyl-β-D-ribofuranosyl)-7H-pyrrolo[2,3-d]pyrimidine; 4-methylamino-7-(2-C-methyl-β-D-ribofuranosyl)-7H-pyrrolo[2,3-d]pyrimidine; 4-dimethylamino-7-(2-C-methyl-β-D-ribofuranosyl)-7H-pyrrolo[2,3-d]pyrimidine; 4-cyclopropylamino-7-(2-C-methyl-β-D-ribofuranosyl)-7H-pyrrolo[2,3-d]pyrimidine; 4-amino-7-(2-C-vinyl-β-D-ribofuranosyl)-7H-pyrrolo[2,3-d]pyrimidine; 4-amino-7-(2-C-hydroxymethyl-β-D-ribofuranosyl)-7H-pyrrolo[2,3-d]pyrimidine; 4-amino-7-(2-C-fluoromethyl-β-D-ribofuranosyl)-7H-pyrrolo[2,3-d]pyrimidine; 4-amino-5-methyl-7-(2-C-methyl-β-D-ribofuranosyl)-7H-pyrrolo[2,3-d]pyrimidine; 4-amino-7-(2-C-methyl-β-D-ribofuranosyl)-7H-pyrrolo[2,3-d]pyrimidine-5-carboxylic acid; 4-amino-5-bromo-7-(2-C-methyl-β-D-ribofuranosyl)-7H-pyrrolo[2,3-d]pyrimidine; 4-amino-5-chloro-7-(2-C-methyl-β-D-ribofuranosyl)-7H-pyrrolo[2,3-d]pyrimidine; 4-amino-5-fluoro-7-(2-C-methyl-β-D-ribofuranosyl)-7H-pyrrolo[2,3-d]pyrimidine; 2,4-diamino-7-(2-C-methyl-β-D-ribofuranosyl)-7H-pyrrolo[2,3-d]pyrimidine; 2-amino-7-(2-C-methyl-β-D-ribofuranosyl)-7H-pyrrolo[2,3-d]pyrimidine; 2-amino-4-cyclopropylamino-7-(2-C-methyl-β-D-ribofuranosyl)-7H-pyrrolo[2,3-d]pyrimidine; 2-amino-7-(2-C-methyl-β-D-ribofuranosyl)-7H-pyrrolo[2,3-d]pyrimidin-4(3H)-one; 4-amino-7-(2-C-ethyl-β-D-ribofuranosyl)-7H-pyrrolo[2,3-d]pyrimidine; 4-amino-7-(2-C,2-O-dimethyl-β-D-ribofuranosyl)-7H-pyrrolo[2,3-d]pyrimidine; 7-(2-C-methyl-β-D-ribofuranosyl)-7H-pyrrolo[2,3-d]pyrimidin-4(3H)-one; 2-amino-5-methyl-7-(2-C,2-O-dimethyl-β-D-ribofuranosyl)-7H-pyrrolo[2,3-d]pyrimidin-4(3H)-one; 4-amino-7-(3-deoxy-2-C-methyl-β-D-ribofuranosyl)-7H-pyrrolo[2,3-d]pyrimidine; 4-amino-7-(3-deoxy-2-C-methyl-β-D-arabinofuranosyl)-7H-pyrrolo[2,3-d]pyrimidine; 4-amino-2-fluoro-7-(2-C-methyl-β-D-ribofuranosyl)-7H-pyrrolo[2,3-d]pyrimidine; 4-amino-7-(3-C-methyl-β-D-ribofuranosyl)-7H-pyrrolo[2,3-d]pyrimidine; 4-amino-7-(3-C-methyl-β-D-xylofuranosyl)-7H-pyrrolo[2,3-d]pyrimidine; 4-amino-7-(2,4-di-C-methyl-β-D-ribofuranosyl)-7H-pyrrolo[2,3-d]pyrimidine; 4-amino-7-(3-deoxy-3-fluoro-2-C-methyl-β-D-ribofuranosyl)-7H-pyrrolo[2,3-d]pyrimidine; and the corresponding 5′-triphosphates; or a pharmaceutically acceptable salt thereof.

The compounds of the present invention may also be combined for the treatment of HCV infection with non-nucleoside inhibitors of HCV polymerase such as those disclosed in U.S. Patent Applciation Publications US 2006/0100262 and US 2009/0048239; International Patent Application Publications WO 01/77091, WO 01/47883, WO 02/04425, WO 02/06246, WO 02/20497, WO 2005/016927 (in particular JTK003), WO 2004/041201, WO 2006/066079, WO 2006/066080, WO 2008/075103, WO 2009/010783 and WO 2009/010785; the content of each is incorporated herein by reference in its entirety.

In one embodiment, additional non-nucleoside HCV NS5B polymerase inhibitors that are used in combination with the present HCV NS5B inhibitors are selected from the following compounds: 14-cyclohexyl-6-[2-(dimethylamino)ethyl]-7-oxo-5,6,7,8-tetrahydroindolo[2,1-a][2,5]benzodiazocine-11-carboxylic acid; 14-cyclohexyl-6-(2-morpholin-4-ylethyl)-5,6,7,8-tetrahydroindolo[2,1-a][2,5]benzodiazocine-11-carboxylic acid; 14-cyclohexyl-6-[2-(dimethylamino)ethyl]-3-methoxy-5,6,7,8-tetrahydroindolo[2,1-a][2,5]benzodiazocine-11-carboxylic acid; 14-cyclohexyl-3-methoxy-6-methyl-5,6,7,8-tetrahydroindolo[2,1-a][2,5]benzodiazocine-11-carboxylic acid; methyl({[(14-cyclohexyl-3-methoxy-6-methyl-5,6,7,8-tetrahydroindolo[2,1-a][2,5]benzodiazocin-11-yl)carbonyl]amino}sulfonyl)acetate; ({[(14-cyclohexyl-3-methoxy-6-methyl-5,6,7,8-tetrahydroindolo[2,1-a][2,5]benzodiazocin-11-yl)carbonyl]amino}sulfonyl)acetic acid; 14-cyclohexyl-N—[(dimethylamino)sulfonyl]-3-methoxy-6-methyl-5,6,7,8-tetrahydroindolo[2,1-a][2,5]benzodiazocine-11-carboxamide; 3-chloro-14-cyclohexyl-6-[2-(dimethylamino)ethyl]-7-oxo-5,6,7,8-tetrahydroindolo[2,1-a][2,5]benzodiazocine 11-carboxylic acid; N-(11-carboxy-14-cyclohexyl-7,8-dihydro-6H-indolo[1,2-e][1,5]benzoxazocin-7-yl)-N,N-dimethylethane-1,2-diaminium bis(trifluoroacetate); 14-cyclohexyl-7,8-dihydro-6H-indolo[1,2-e][1,5]benzoxazocine-11-carboxylic acid; 14-cyclohexyl-6-methyl-7-oxo-5,6,7,8-tetrahydroindolo[2,1-a][2,5]benzodiazocine-11-carboxylic acid; 14-cyclohexyl-3-methoxy-6-methyl-7-oxo-5,6,7,8-tetrahydroindolo[2,1-a][2,5]benzodiazocine-11-carboxylic acid; 14-cyclohexyl-642-(dimethylamino)ethyl]-3-methoxy-7-oxo-5,6,7,8-tetrahydroindolo[2,1-a][2,5]benzodiazocine-11-carboxylic acid; 14-cyclohexyl-6-[3-(dimethylamino)propyl]-7-oxo-5,6,7,8-tetrahydroindolo[2,1-a][2,5]benzodiazocine-11-carboxylic acid; 14-cyclohexyl-7-oxo-6-(2-piperidin-1-ylethyl)-5,6,7,8-tetrahydroindolo[2,1-a][2,5]benzodiazocine-11-carboxylic acid; 14-cyclohexyl-6-(2-morpholin-4-ylethyl)-7-oxo-5,6,7,8-tetrahydroindolo[2,1-a][2,5]benzodiazocine-11-carboxylic acid; 14-cyclohexyl-6-[2-(diethylamino)ethyl]-7-oxo-5,6,7,8-tetrahydroindolo[2,1-a][2,5]benzodiazocine-11-carboxylic acid; 14-cyclohexyl-6-(1-methylpiperidin-4-yl)-7-oxo-5,6,7,8-tetrahydroindolo[2,1-a][2,5]benzodiazocine-11-carboxylic acid; 14-cyclohexyl-N—[(dimethylamino)sulfonyl]-7-oxo-6-(2-piperidin-1-ylethyl)-5,6,7,8-tetrahydroindolo[2,1-a][2,5]benzodiazocine-11-carboxamide; 14-cyclohexyl-642-(dimethylamino)ethyl]-N—[(dimethylamino)sulfonyl]-7-oxo-5,6,7,8-tetrahydroindolo[2,1-a][2,5]benzodiazocine-11-carboxamide; 14-cyclopentyl-6-[2-(dimethylamino)ethyl]-7-oxo-5,6,7,8-tetrahydroindolo[2,1-a][2,5]benzodiazocine-11-carboxylic acid; 14-cyclohexyl-5,6,7,8-tetrahydroindolo[2,1-a][2,5]benzodiazocine-11-carboxylic acid; 6-allyl-14-cyclohexyl-3-methoxy-5,6,7,8-tetrahydroindolo[2,1-a][2,5]benzodiazocine-11-carboxylic acid; 14-cyclopentyl-6-[2-(dimethylamino)ethyl]-5,6,7,8-tetrahydroindolo[2,1-a][2,5]benzodiazocine-11-carboxylic acid; 14-cyclohexyl-6-[2-(dimethylamino)ethyl]-5,6,7,8-tetrahydroindolo[2,1-a][2,5]benzodiazocine-11-carboxylic acid; 13-cyclohexyl-5-methyl-4,5,6,7-tetrahydrofuro[3′,2′:6,7][1,4]diazocino[1,8-a]indole-10-carboxylic acid; 15-cyclohexyl-6,2-(dimethylamino)ethyl]-7-oxo-6,7,8,9-tetrahydro-5H-indolo[2,1-a][2,6]benzodiazonine-12-carboxylic acid; 15-cyclohexyl-8-oxo-6,7,8,9-tetrahydro-5H-indolo[2,1-a][2,5]benzodiazonine-12-carboxylic acid; 13-cyclohexyl-6-oxo-6,7-dihydro-5H-indolo[1,2-d][1,4]benzodiazepine-10-carboxylic acid; and pharmaceutically acceptable salts thereof.

In another embodiment, the present HCV NS5B polymerase inhibitors are used in combination with non-nucleoside HCV NS5A inhibitors and pharmaceutically acceptable salts thereof.

The HCV NS5B inhibitory activity of the present compounds may be tested using assays known in the art. The HCV NS5B polymerase inhibitors described herein have activities in a genotype 1b replicon assay as described in the Examples. The assay is performed by incubating a replicon harboring cell-line in the presence of inhibitor for a set period of time and measuring the effect of the inhibitor on HCV replicon replication either directly by quantifying replicon RNA level, or indirectly by measuring enzymatic activity of a co-encoded reporter enzyme such as luciferase or β-lactamase. By performing a series of such measurements at different inhibitor concentrations, the effective inhibitory concentration of the inhibitor (EC₅₀ or EC₉₀) is determined. See Jan M. Vrolijk et al., A replicons-based bioassay for the measurement of interferons in patients with chronic hepatitis C, 110 J. VIROLOGICAL METHODS 201 (2003). Such assays may also be run in an automated format for high through-put screening. See Paul Zuck et al., A cell-based β-lactamase reporter gene assay for the identification of inhibitors of hepatitis C virus replication, 334 ANALYTICAL BIOCHEMISTRY 344 (2004).

The present invention also includes processes for making Compounds of Formula (I). The compounds of the present invention can be readily prepared according to the following reaction schemes and examples, or modifications thereof, using readily available starting materials, reagents and conventional synthesis procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples. Unless otherwise indicated, all variables are as defined above. The following reaction schemes and examples serve only to illustrate the invention and its practice.

### General Schemes

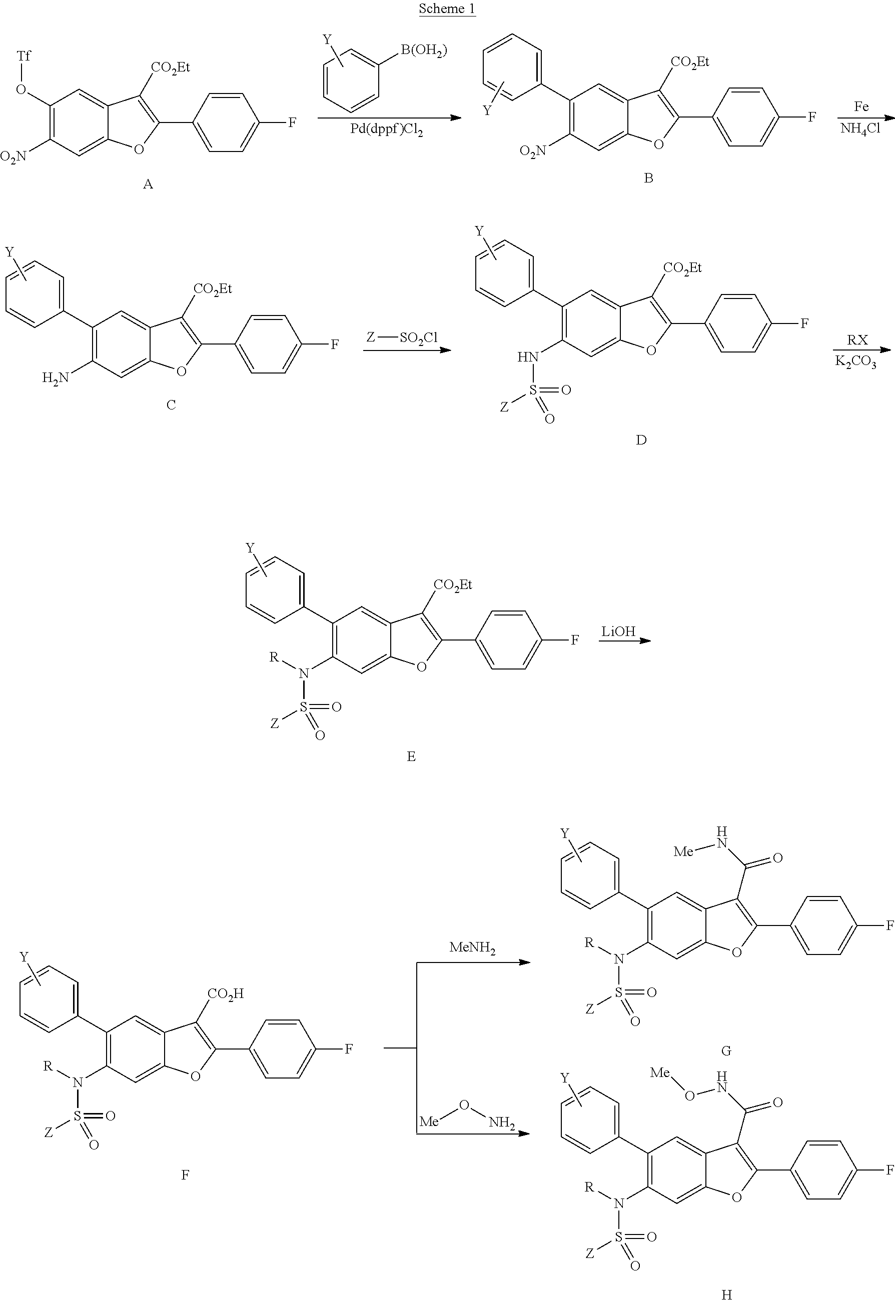

This scheme describes the preparation of compounds with the general structure of G and H. Starting from compound A (obtained according to procedure in WO 2004/041201 A2), coupling with a substituted or unsubstituted phenylboronic acid catalyzed by a transition metal, in this case Pd(dppf)Cl₂, furnishes compounds of the general structure B. This type of transition-metal-mediated cross-coupling is common and there are numerous conditions that one skilled in the art can use to execute such a transformation. Compounds of type C are next generated by reduction of the nitro group in compound B, which can be accomplished by exposure to common reducing conditions, in this case treatment by Fe in NH₄Cl solution under reflux. The amino group in compounds C is then sulfonylated with a sulphonyl chloride to give compounds of type D. The sulfonamide D can be coupled with an alkylating agent (an alkyl halide for example) in the presence of a suitable base, such as potassium carbonate, to provide compounds E. The ester functionality in compounds E is readily hydrolyzed by aqueous base to afford compounds F. The carboxylic acid of compound F was condensed with methanamine or O-methylhydroxylamine using common amide-forming reagents such as EDCI and HOBT to give compounds G or compounds H.

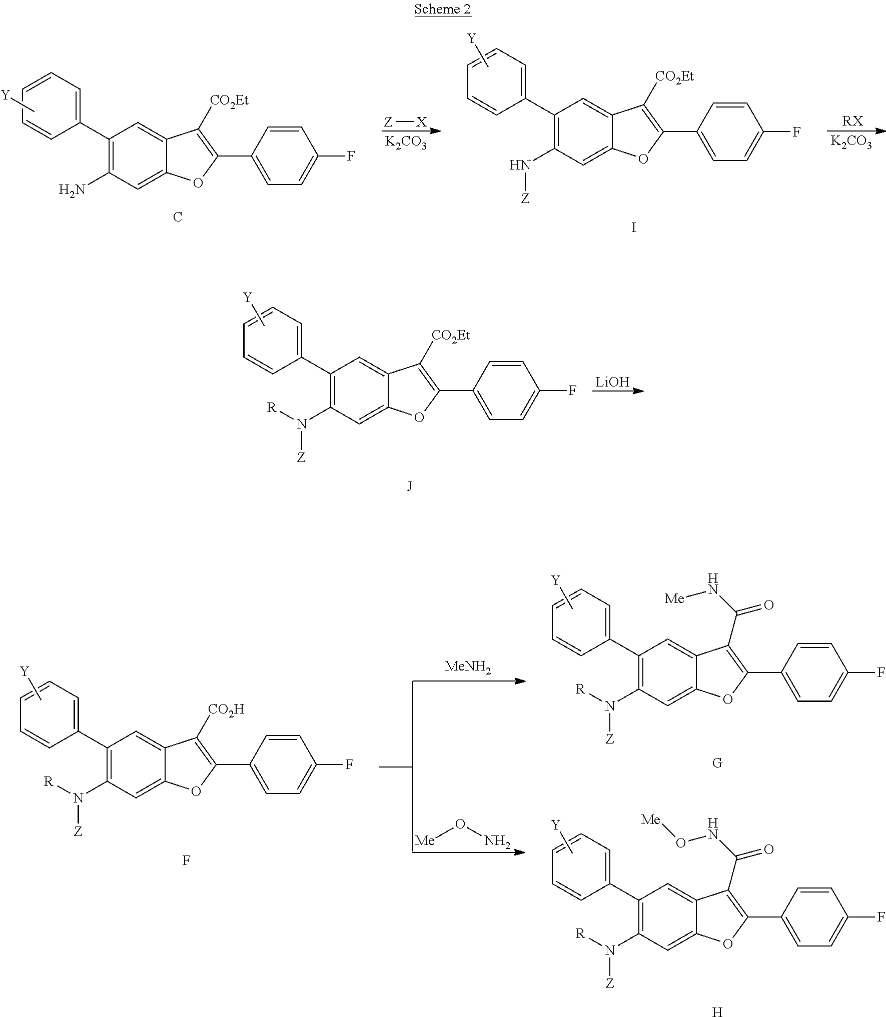

Compound C can be coupled with an alkylating agent (an alkyl halide for example) in the presence of a suitable base, such as potassium carbonate, to provide compounds I where Z represents an alkylated aniline. Alternatively C may be condensed with substituted carboxylic acid in the presence of coupling reagents, such as EDCI and HOBT, to afford compounds I where Z represents a substituted amide. Compounds J may be obtained from compounds I by further N-alkylation or N-acylation reaction. Compounds of general structure I or J are hydrolyzed by aqueous hydroxide to provide compounds F. The carboxylic acid of compound F may be condensed with an amine as shown in Scheme 1 to provide target compounds of general structure G and H.

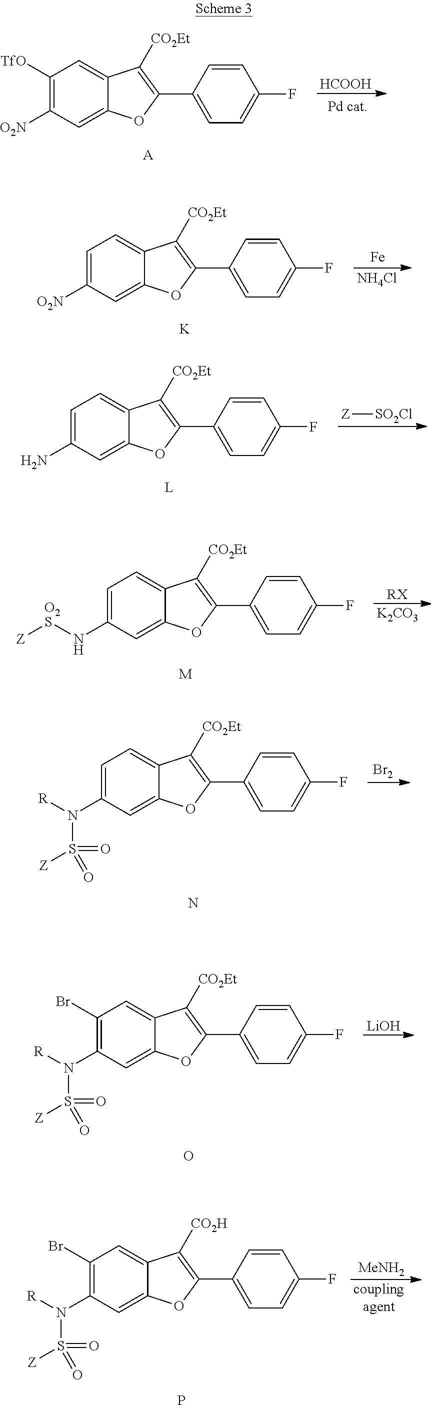

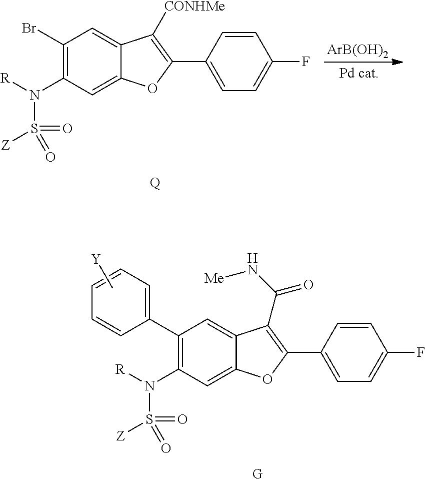

Compound A may be reduced by a catalyst in the presence of a hydrogen source (for example, Pd in the presence of formic acid) to afford compound K. Further reduction of K provides aniline L. The amino group of compound L is reacted with sulfonyl chloride to afford compound M, which can be further N-alkylated with a wide variety of alkylating agents in the presence of a suitable base, such as potassium carbonate, to provide compound N. Halogenation of compound N, in this case bromination with FeCl₃ and Br₂ in anhydrous CCl₄ gives compound O. Compounds of general structure O are hydrolyzed by aqueous hydroxide to provide compounds P. The carboxylic acid of compound P may be condensed with an amine as shown in Scheme 1 to provide compounds of general structure Q. Transition metal mediated coupling of compounds Q with a boronic acid (alternatively alkyl tin, silicon, or other types of coupling partners may be used) provides the target compounds of general structure G.

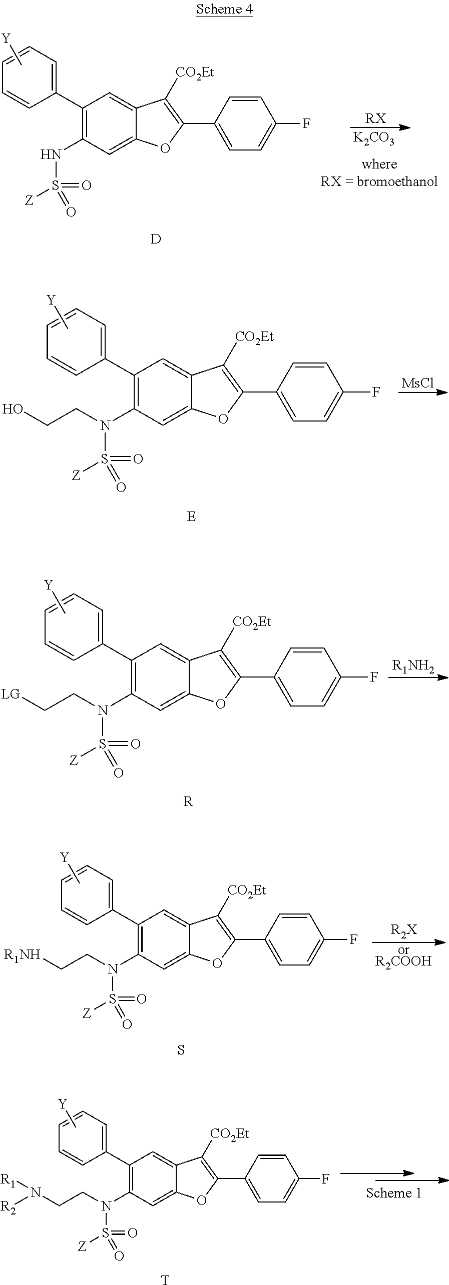

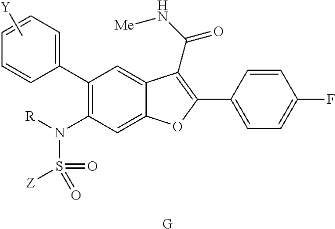

Compounds E that possess a hydroxyl group may be obtained from compounds D by reacting with 2-bromo ethanol. The hydroxyl group E can be converted to a leaving group (by reaction with MsCl for example) to afford compound R. Compound R may be treated with nucleophilic reagents such as an amine in the presence of a suitable base, such as triethylamine, to afford compound S. Compounds T can then be obtained from compound S by further N-alkylation or N-acylation. Compounds of structure T are readily converted to the target structures G following the general procedure described in Scheme 1.

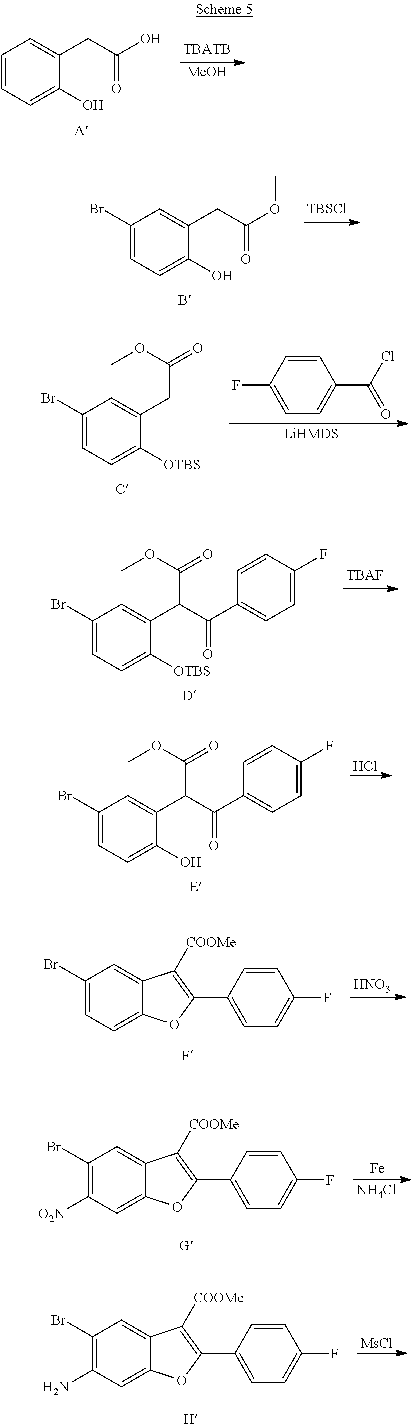

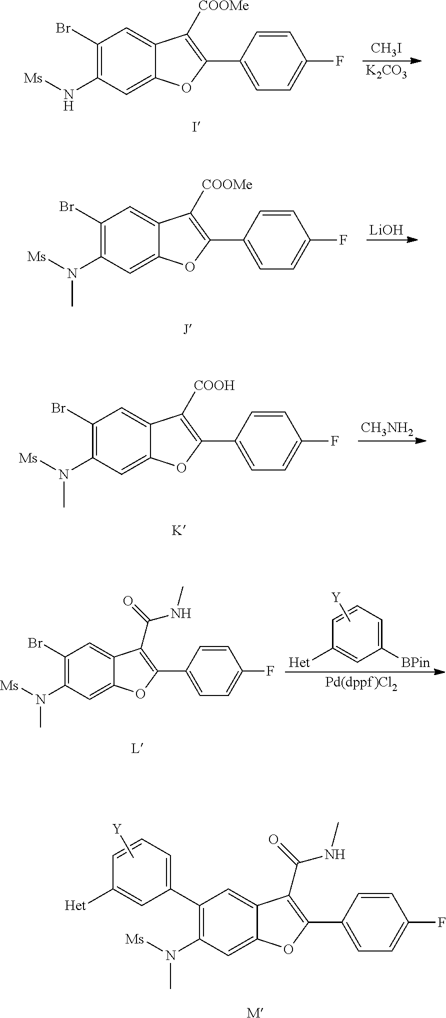

This scheme describes the preparation of compounds with the general structure of M′. Starting from compound A′, bromating and esterifying with TBATB in MeOH to afford compound B′. Protecting the phenol group of B′ with TBSCl provides compound C′, which can be C-acylated with 4-fluorobenzoyl chloride to give compound D′. After de-protection with TBAF and cyclizing by concentrated HCl, compound D′ affords compound E′ and F′ sequentially. Compound F′ can be converted to compound G′ by treated with fuming HNO₃. Compound H′ is generated by reduction of the nitro group in compound G′, and the amino group in compound H′ is then sulfonylated with MsCl to furnish compound I′. The sulfonamide I′ can be coupled with MeI in the presence of potassium carbonate to provide compound J′. The ester functionality in compound J′ is readily hydrolyzed by aqueous base to afford compound K′. The carboxylic acid of compound K′ was condensed with methanamine using common amide forming reagents such as EDCI and HOBT to give compound L′. Transition metal mediated coupling of compound L′ with a meta-heterocycle-substituted phenyl boronic ester (alternatively boronic acid, alkyl tin, silicon, or other types of coupling partners may be used) provides the target compounds of general structure M′.

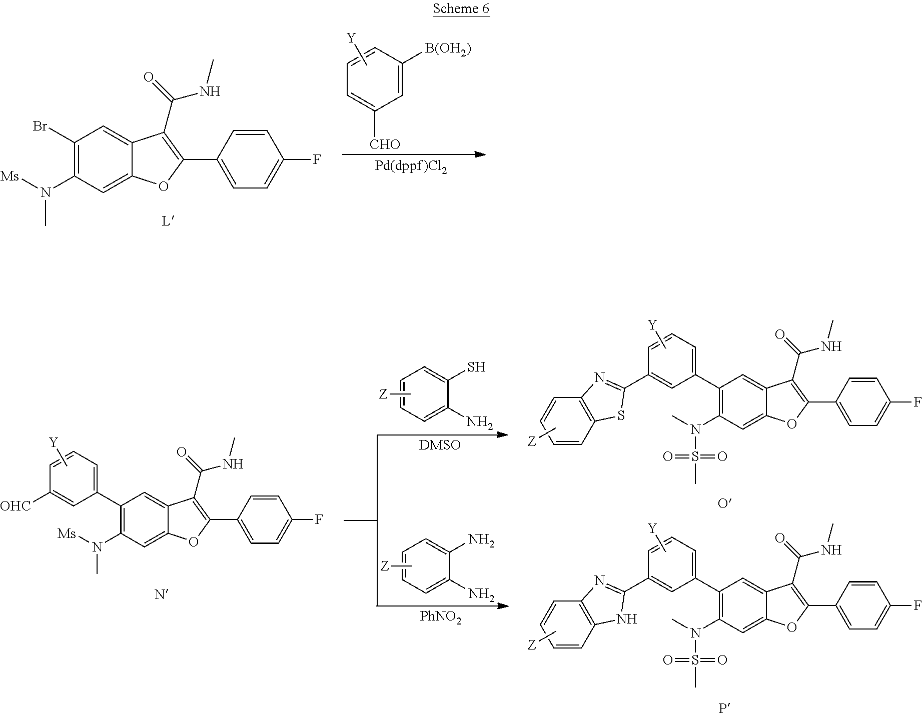

Coupling compound L′ with a substituted or unsubstituted 3-formylphenylboronic acid catalyzed by a transition metal, in this case Pd(dppf)Cl₂, furnishes compounds of the general structure N′. Compounds of type N′ were cyclized with ortho-amino anilines or ortho-amino thiophenols to provide the target compounds of general structure O′ or P′.

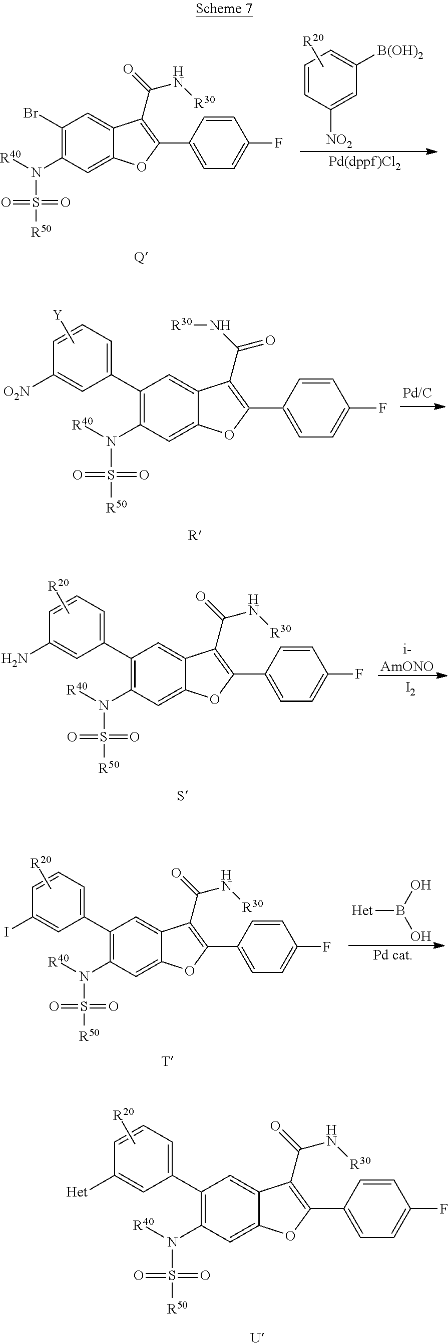

This scheme describes a method useful for making the compounds of formula U′, which correspond to the Compounds of Formula (II) wherein Het is a heterocyclyl or heteroaryl group; R⁶⁰ is para-F; and R²⁰, R³⁰, R⁴⁰ and R⁵⁰ are defined above for the Compounds of Formula (II).

A compound of formula Q′ can be coupled with a substituted or unsubstituted 3-nitrophenylboronic acid catalyzed by a transition metal, in this case Pd(dppf)Cl₂, to provide the compounds of formula R′. Compounds of formula R′ can then be hydrogenated to provide the amino compounds of formula S′, which are reacted with i-AmONO/I₂, to provide the iodo compounds of formula T′. Transition metal mediated coupling of T′ with a heterocyclic boronic acid (alternatively boronic ester, alkyl tin, silicon, or other types of coupling partners may be used) provides the target compounds of formula U′.

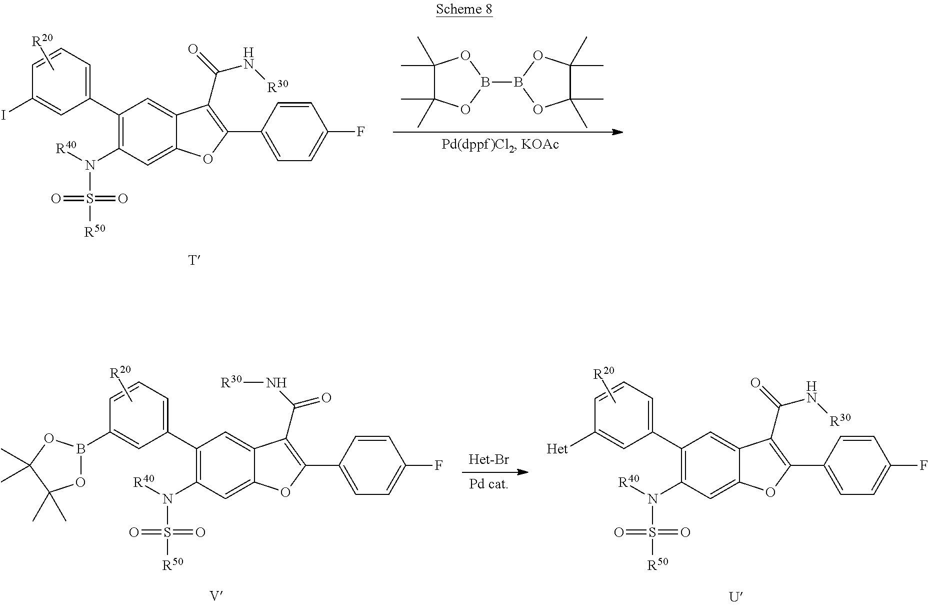

This scheme describes an alternate useful for making the compounds of formula U′, which correspond to the Compounds of Formula (II) wherein Het is a heterocyclyl or heteroaryl group; R⁶⁰ is para-F; and R²⁰, R³⁰, R⁴⁰ and R⁵⁰ are defined above for the Compounds of Formula (II).

An iodo compound of formula T′ can be converted to boronic ester compounds of formula V′ in the presence of Pd(dppf)Cl₂. A compound of formula V′ can then be coupled with and aryl bromide or heterocyclic bromide to provide the compounds of formula U′.

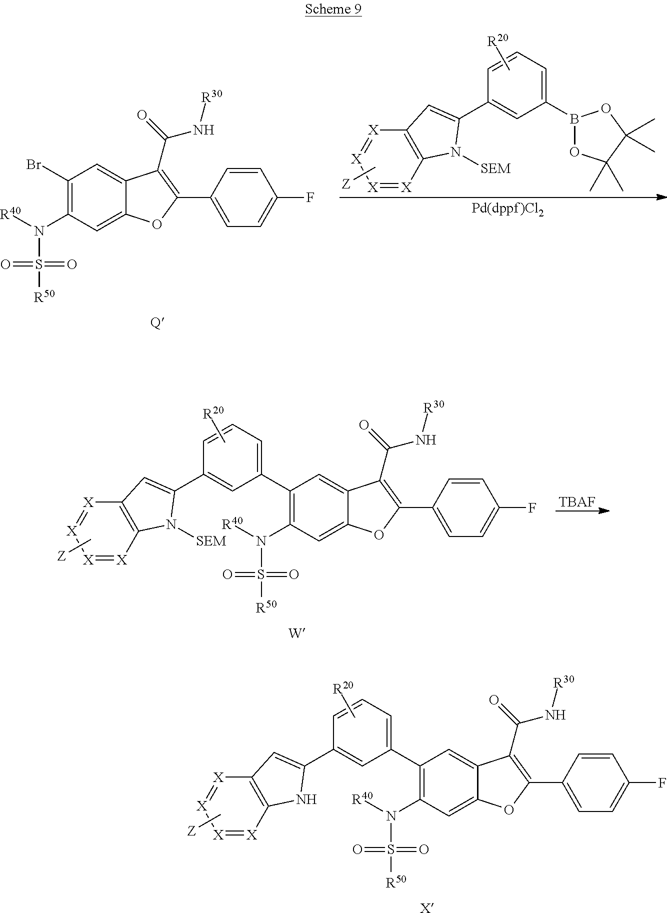

This scheme describes a method useful for making the compounds of formula W, which correspond to the Compounds of Formula (II) wherein R¹⁰ is indole or other bicyclic pyrrole derivative; R⁶⁰ is para-F; and R²⁰, R³⁰, R⁴⁰ and R⁵⁰ are defined above for the Compounds of Formula (II).

A transition metal-mediated coupling of a compound of a bromo compound of formula Q′ with a heterocycle substituted phenyl boronic ester (alternatively boronic acid, alkyl tin, silicon, or other types of coupling partners may be used) provides the compounds of formula W′. The SEM protecting group of a compound of formula W′ can subsequently be deproteted using TBAF to provide the compounds of formula X′.

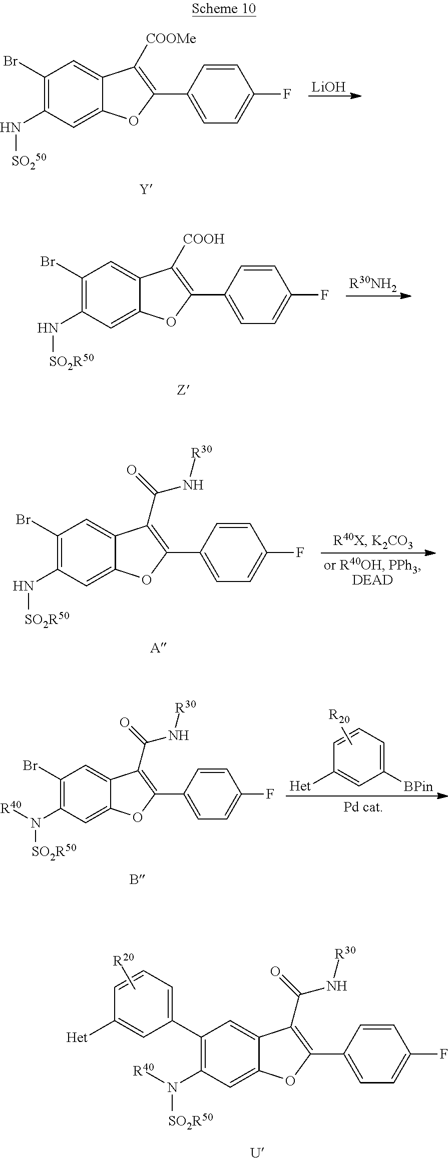

This scheme describes an alternate method useful for making the compounds of formula U′, which correspond to the Compounds of Formula (II) wherein Het is a heterocyclyl or heteroaryl group; R⁶⁰ is para-F; and R²⁰, R³⁰, R⁴⁰ and R⁵⁰ are defined above for the Compounds of Formula (II).

The ester group of a compound of formula Y′ can be hydrolyzed using aqueous base to provide a compound of formula Z′. The carboxylic acid moiety of Z′ can then be condensed with an amine of formula R³⁰NH₂ using common amide forming reagents, such as EDCI and HOBT, to provide the compounds of formula A″. The sulfonamide group of A″ can then be coupled with a reagent of formula R⁴⁰X in the presence of potassium carbonate or with a regent of formula R⁴⁰OH in the presence of PPh₃ and DEAD to provide compounds of formula B″. Transition metal mediated coupling of a compound of formula B″ with a heterocycle-substituted phenyl boronic ester (alternatively boronic acid, alkyl tin, silicon, or other types of coupling partners may be used) provides the compounds of formula U′.

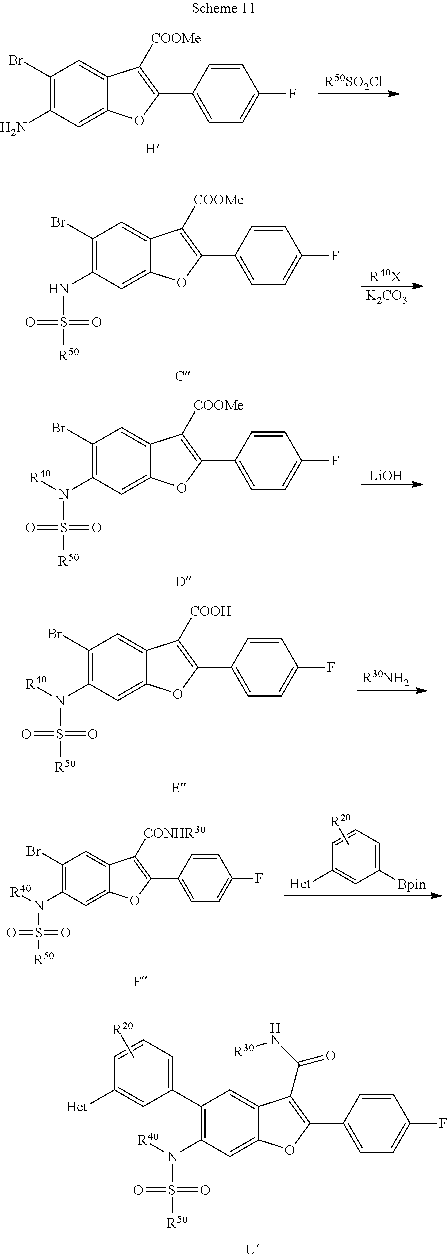

This scheme describes yet another alternate method useful for making the compounds of formula U′, which correspond to the Compounds of Formula (I) wherein Het is a heterocyclyl or heteroaryl group; R⁶⁰ is para-F; and R²⁰, R³⁰, R⁴⁰ and R⁵⁰ are defined above for the Compounds of Formula (I).

The amino group of a compound of formula H′ can be sulfonylated using a reagent of formula R⁵⁰SO₂Cl to provide the sulfonamide compounds of formula C″. A compound of formula C″ can then be coupled with a reactant of formula R⁴⁰X in the presence of potassium carbonate to provide the compounds of formula D″. The ester moiety of the compounds of formula D″ can be readily hydrolyzed using aqueous base to provide the compounds of formula E″. The carboxylic acid group of E″ is then condensed with an amine of formula R³⁰NH₂ using common amide forming reagents, such as EDCI and HOBT, to provide the compounds of formula to F″. Transition metal mediated coupling of a compound of formula F″ with a heterocycle-substituted phenyl boronic ester (alternatively boronic acid, alkyl tin, silicon, or other types of coupling partners may be used) provides the compounds of formula U′.

### LIST OF ABBREVIATIONS

AcOH Acetic acid
i-AmONO iso-Amylnitrite
n-BuLi n-butyllithium
Bu₃N Tributylamine
CCl₄ Carbon tetrachloride or tetrachloromethane
CDCl₃ Deuterated chloroform
MeCN, CH₃CN Acetonitrile
MeNH₂, CH₃NH₂ Methylamine
MeONH₂, CH₃ONH₂ Methoxyamine
Cs₂CO₃ Cesium carbonate
DCM Dichloromethane
DEAD Diethylazodicarboxylate
DMF Dimethylformamide
DMSO Dimethylsulfoxide
EDCI N-(3-Dimethylaminopropyl)-N′-ethylcarbodiimide (also EDC)
Et₃N Triethylamine
EtOAc Ethyl acetate
EtOH Ethanol
EtOOCCl, CICOOEt Ethyl chloroformate
HOBT 1-Hydroxy benzotriazole
¹H-NMR Proton Nuclear Magnetic Resonance
HPLC High Performance Liquid Chromatography
KOAc Potassium acetate
K₃PO₄ Potassium Phosphate
LDA Lithium diisopropylamide
LiHMDS Lithium bis(trimethylsilyl)amide
LiOH.H₂O Lithium hydroxide monohydrate
MeNH₂ Methanamine
MeCN Acetonitrile
MeOD Deuterated methanol
MeOH Methanol
MeONH₂ Methoxyamine
MS Mass spectroscopy
Ms Methanesulfonyl (mesyl)
MsCl Methanesulfonyl chloride
NBS N-Bromosuccinimide
NCS N-Chlorosuccinimide
PE Petroleum ether
PPh₃ Triphenylphosphine
Pd—C, Pd/C Palladium on carbon
Pd(dppf)Cl₂ 1,1′-bis(diphenylphosphino)ferrocene-palladium(II)dichloride
Pd(PPh₃)₂Cl₂ 1,1′-bis(tetrakis(triphenylphosphine))palladium(II)dichloride
Pd(PPh₃)₄ Tetrakis(triphenylphospine)palladium(0)
Ph Phenyl
PhB(OH)₂ Phenylboronic acid
PhNO₂ Nitrobenzene
PhSO₂Cl Benzenesulfonyl chloride
i-PrNH₂ Diisopropylamine
Py Pyridine
RT Room temperature, approximately 25° C.
SEM 2-(Trimethylsilyl)ethoxymethyl
TBAF Tetrabutyl ammonium fluoride
TBATB Tetrabutylammonium tribromide
TBS Tert-butyldimethylsilyl
TBSCl Tert-butyldimethylsilylchloride
Tf Trifluoromethanesulfonate (triflate)
THF Tetrahydrofuran
TLC Thin layer chromatography

### EXAMPLES

### Example 1

### 2-(4-fluorophenyl)-N-methyl-6-[methyl(methylsulfonyl)amino]-5-phenyl-1-benzofuran-3-carboxamide

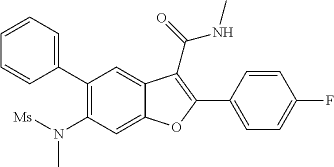

### Step 1: ethyl 2-(4-fluorophenyl)-6-nitro-5-phenyl-1-benzofuran-3-carboxylate

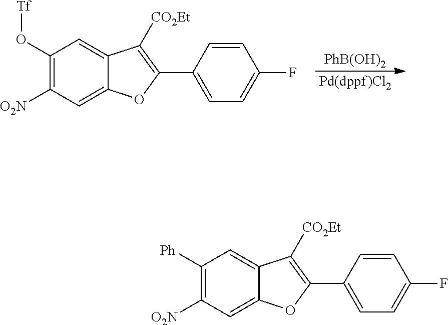

Phenylboronic acid (100 mg, 0.8 mmol) and K₃PO₄.3H₂O (119 mg, 0.8 mmol) were added to a suspension of ethyl 2-(4-fluorophenyl)-6-nitro-5-{[(trifluoromethyl)sulfonyl]oxy}-1-benzofuran-3-carboxylate (obtained according to procedure in WO 2004/041201 A2, 200 mg, 0.4 mmol) in dioxane (2 mL) and DMF (2 mL) under N₂ protection. Then, Pd(dppf)Cl₂ (5 mg, 0.08 mmol) was added to the mixture under N₂ protection. The reaction mixture was heated to 90° C. for 30 minutes. After cooling, the mixture was diluted with H₂O and extracted with EtOAc. The combined organic phases were washed with brine, dried over Na₂SO₄, filtered and evaporated. The crude product was purified by prep-TLC to give pure ethyl 2-(4-fluorophenyl)-6-nitro-5-phenyl-1-benzofuran-3-carboxylate (35 mg, yield: 23%).

¹H-NMR (400 MHz, CDCl₃) δ 7.88˜7.98 (m, 2H), 7.62 (s, 1H), 7.44˜7.48 (m, 4H), 7.32˜7.38 (m, 1H), 7.06˜7.12 (m, 2H), 6.78 (s, 1H), 4.29˜4.35 (m, 2H), 1.27˜1.30 (m, 3H)

### Step 2: ethyl 6-amino-2-(4-fluorophenyl)-5-phenyl-1-benzofuran-3-carboxylate

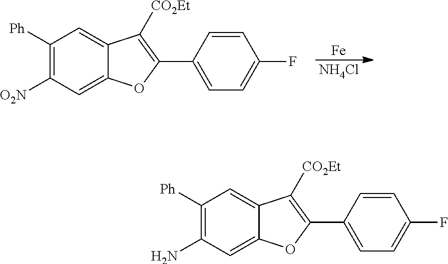

A mixture of ethyl 2-(4-fluorophenyl)-6-nitro-5-phenyl-1-benzofuran-3-carboxylate (110 mg, 0.27 mmol), Fe (120 mg, 2.16 mmol) and NH₄Cl (217 mg, 4.05 mmol) in H₂O/MeOH/THF (1 mL/1 mL/1 mL) was refluxed for 4 hours. Then, H₂O was added to quench the reaction, and the mixture was extracted with EtOAc. After washing with brine and dried, the solvent was removed by distillation. The pure product of ethyl 6-amino-2-(4-fluorophenyl)-5-phenyl-1-benzofuran-3-carboxylate was obtained (85 mg, yield: 85%) by prep-TLC.

¹H-NMR (400 MHz, CDCl₃) δ 8.00˜8.03 (m, 2H), 7.85 (d, J=7.2 Hz, 2H), 7.45˜7.49 (m, 3H), 7.29˜7.32 (m, 2H), 7.10˜7.14 (m, 2H), 4.29˜4.35 (m, 2H), 1.27˜1.30 (m, 3H).

### Step 3: ethyl 2-(4-fluorophenyl)-6-[(methylsulfonyl)amino]-5-phenyl-1-benzofuran-3-carboxylate

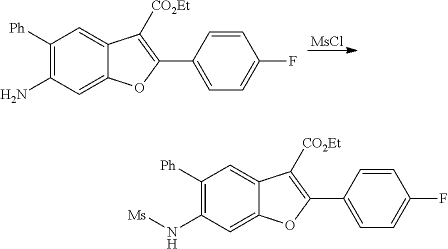

MsCl (66 mg, 0.6 mmol) was added to a solution of the product of Step 2 (85 mg, 0.23 mmol) and pyridine (73 mg, 0.92 mmol) in dry DCM (2 mL). The reaction mixture was stirred overnight at RT. After dilution with H₂O and extraction with DCM, the organic layer was washed with brine, dried over Na₂SO₄ and filtered, and the solvent was evaporated under reduced pressure. The crude product was purified by prep-TLC to give ethyl 2-(4-fluorophenyl)-6-[(methylsulfonyl)amino]-5-phenyl-1-benzofuran-3-carboxylate (90 mg, yield: 86%).

¹H-NMR (400 MHz, CDCl₃) δ 8.00˜8.03 (m, 2H), 7.85 (d, J=7.2 Hz, 2H), 7.45˜7.49 (m, 3H), 7.29˜7.32 (m, 2H), 7.10˜7.14 (m, 2H), 6.50 (s, 1H), 4.29˜4.35 (m, 2H), 2.80 (s, 3H), 1.27˜1.30 (m, 3H).

### Step 4: ethyl 2-(4-fluorophenyl)-6-[methyl(methylsulfonyl)amino]-5-phenyl-1-benzofuran-3-carboxylate

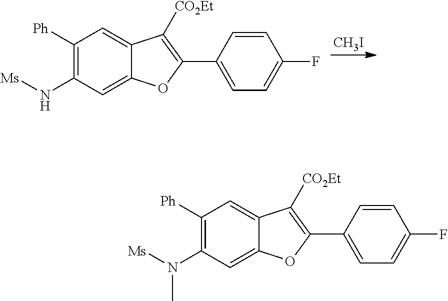

NaH (60% in oil, 20 mg, 0.5 mmol) and CH₃I (85 mg, 0.6 mmol) were added to a solution of the product of Step 3 (90 mg, 0.2 mmol) in dry DMF under N₂ protection. The mixture was stirred overnight at RT, and then ice-cold diluted AcOH was added to the mixture. After extraction with EtOAc, the organic solvent was washed with brine, dried over Na₂SO₄, filtered and the solvent was evaporated under reduced pressure. The crude product was purified by prep-TLC to give ethyl 2-(4-fluorophenyl)-6-[methyl(methylsulfonyl)amino]-5-phenyl-1-benzofuran-3-carboxylate (78 mg, yield: 84%).

¹H-NMR (400 MHz, CDCl₃) δ 8.00˜8.02 (m, 2H), 7.97˜7.98 (m, 1H), 7.55˜7.56 (m, 1H), 7.39˜7.40 (m, 4H), 7.32˜7.34 (m, 1H), 7.11˜7.15 (m, 2H), 4.32 (q, J=7.2 Hz, 2H), 3.11 (s, 3H), 2.45 (s, 3H), 1.26˜1.30 (t, J=6.8 Hz, 3H).

### Step 5: 2-(4-fluorophenyl)-6-[methyl(methylsulfonyl)amino]-5-phenyl-1-benzofuran-3-carboxylic acid

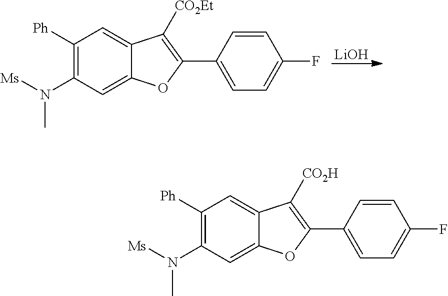

The product of Step 4 (78 mg, 0.17 mmol) was dissolved in THF (2 mL) and H₂O (2 mL). Then, LiOH (71 mg, 1.7 mmol) was added to the solution, and the mixture was stirred at RT overnight. After acidification with HCl (1 N) and extraction with EtOAc, the combined organic phases were washed with brine, dried over Na₂SO₄, filtered and evaporated to give the product of 2-(4-fluorophenyl)-6-[methyl(methylsulfonyl)amino]-5-phenyl-1-benzofuran-3-carboxylic acid (50 mg, yield: 67%). It was used for the next step without further purification.

### Step 6: 2-(4-fluorophenyl)-N-methyl-6-[methyl(methylsulfonyl)amino]-5-phenyl-1-benzofuran-3-carboxamide

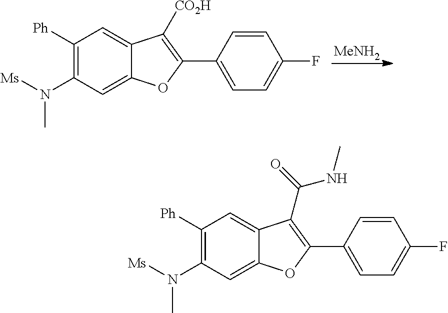

The product of Step 5 (50 mg, 0.11 mmol), HOBT (24.5 mg, 0.16 mmol) and EDCI (52 mg, 0.27 mmol) were dissolved in dry DMF (2 mL). The resulting solution was stirred for 30 minutes. Then, methanamine (HCl salt, 14 mg, 0.44 mmol) and Et₃N (50 mg, 0.47 mmol) were added to the mixture. After stirring overnight, the mixture was diluted with H₂O and extracted with EtOAc. The combined organic phases were washed with brine, dried over Na₂SO₄, filtered and evaporated. The crude product was purified by prep-TLC to give pure 2-(4-fluorophenyl)-N-methyl-6-[methyl(methylsulfonyl)amino]-5-phenyl-1-benzofuran-3-carboxamide (20 mg, yield: 40%).

¹H-NMR (400 MHz, CDCl₃) δ 7.92˜7.96 (m, 2H), 7.59 (s, 1H), 7.52˜7.54 (m, 1H), 7.29˜7.47 (m, 5H), 7.11˜7.18 (m, 2H), 5.84 (s, 1H), 3.25 (s, 3H), 2.98 (d, J=7.2 Hz, 3H), 2.61 (s, 3H).

### Examples 2-6

Examples 2 through 6 were prepared according to the general procedures of Example 1.

| Ex- | | | ¹H-NMR (400 MHz, | MS |
|---|---|---|---|---|
| ample | Structure | Name | CDCl₃) δ | (M + H)⁺ |
| 2 | 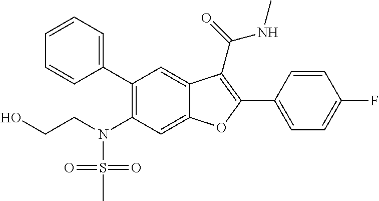 | 2-(4-fluorophenyl)-6- [(2-hydroxyethyl) (methylsulfonyl) amino]-N-methyl-5- phenyl-1-benzofuran- 3-carboxamide | 7.86~7.90 (m, 2H), 7.72 (s, 1H), 7.59 (s, 1H), 7.47~7.50 (m, 2H), 7.32~7.40 (m, 3H), 7.10~7.16 (m, 2H), 5.80 (s, 1H), 3.28~3.47 (m, 4H), 2.90 (s, 6H). | 483 |
| 3 | 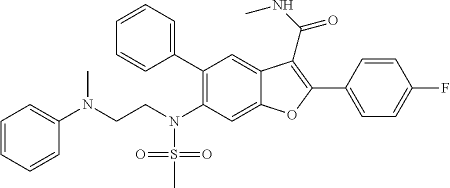 | 2-(4-fluorophenyl)-N- methyl-6-[{2- [methyl(phenyl) amino]ethyl} (methylsulfonyl) amino]-5-phenyl-1- benzofuran-3- carboxamide | 7.90~7.91 (m, 2H), 7.74 (s, 1H), 7.51 (s, 1H), 7.31~7.43 (m, 5H), 7.08~7.18 (m, 4H), 6.60~6.63 (m, 1H), 6.48~6.50 (m, 2H), 5.78 (s, 1H), 3.24~3.41 (m, 4H), 2.92 (d, J = 4.8 Hz, 3H), 2.74 (s, 3H), 2.70 (s, 3H). | 572 |
| 4 | 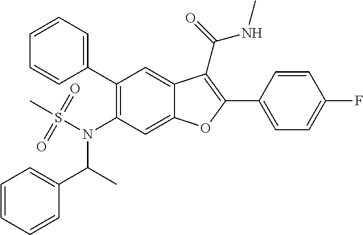 | 2-(4-fluorophenyl)-N- methyl-6- [(methylsulfonyl) (1- phenylethyl) amino]- 5-phenyl-1- benzofuran-3- carboxamide | 7.86~7.93 (m, 2H), 7.65 (d, J = 2 Hz, 2H), 7.36~7.60 (m, 5H), 7.08~7.26 (m, 5H), 6.99~7.05 (m, 2H), 6.87 (s, 1H), 2.90 (t, J = 5.2 Hz, 3H), 2.87 (t, J = 6 Hz, 3H), 1.30 (t, J = 6.8 Hz, 3H). | 543 |
| 5 | 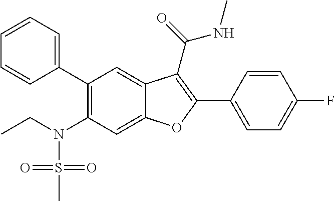 | 6-[ethyl (methylsulfonyl) amino]-2-(4- fluorophenyl)-N- methyl-5-phenyl-1- benzofuran-3- carboxamide | 7.88~7.92 (m, 2H), 7.77 (s, 1H), 7.5 (s, 1H), 7.32~7.42 (m, 5H), 7.14 (t, J = 8.8 Hz, 2H), 5.79 (s, 1H), 3.21~3.42 (m, 2H), 2.91 (d, J = 4.l Hz, 3H), 2.67 (s, 3H), 1.01 (t, J = 7.2 Hz, 3H). | 467 |
| 6 | 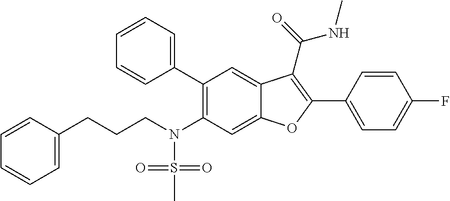 | 2-(4-fluorophenyl)-N- methyl-6- [(methylsulfonyl) (3- phenylpropyl) amino]- 5-phenyl-1- benzofuran-3- carboxamide | 7.86~7.90 (m, 2H), 7.67 (s, 1H), 7.48 (s, 1H), 7.31~7.35 (m, 5H), 7.09~7.16 (m, 5H), 6.94 (d, J = 7.2 Hz, 2H), 5.10 (d, J = 4.4 Hz, 1H), 3.37~3.41 (m, 2H), 2.89 (d, J = 5.2 Hz, 3H), 2.69 (s, 3H), 2.36 (d, J = 5.6 Hz, 2H), 1.56 (d, J = 11.2 Hz, 2H). | 557 |

### Example 7

### 2-(4-fluorophenyl)-N-methoxy-6-[methyl(methylsulfonyl)amino]-5-phenyl-1-benzofuran-3-carboxamide

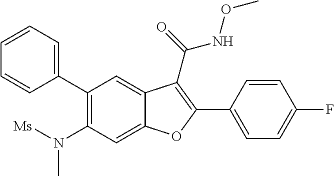

### Steps 1-5

Steps 1-5 were performed in accordance with Example 1, Steps 1-5.

### Step 6: 2-(4-fluorophenyl)-N-methoxy-6-[methyl(methylsulfonyl)amino]-5-phenyl-1-benzofuran-3-carboxamide

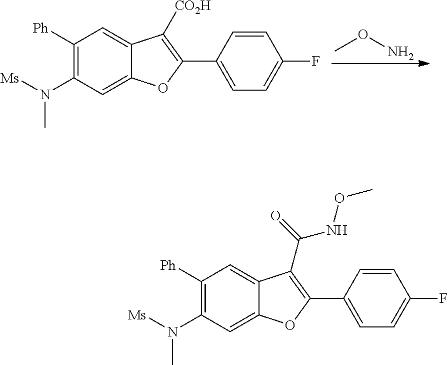

The product of Step 5 (50 mg, 0.11 mmol), HOBT (24.5 mg, 0.16 mmol) and EDCI (52 mg, 0.27 mmol) were dissolved in dry DMF (2 mL). The resulting solution was stirred for 30 minutes. Then, O-methylhydroxylamine (HCl salt, 36 mg, 0.44 mmol) and Et₃N (50 mg, 0.47 mmol) were added to the mixture. After stirred overnight, the mixture was diluted with H₂O and extracted with EtOAc. The combined organic phases were washed with brine, dried over Na₂SO₄, filtered and evaporated. The crude product was purified by prep-TLC to give pure product (20 mg, yield: 40%).

¹H-NMR (400 MHz, CDCl₃) δ 8.26˜8.27 (m, 1H), 7.70˜7.87 (m, 2H), 7.56 (s, 1H), 7.41 (s, 1H), 7.34˜7.39 (m, 5H), 7.12˜7.16 (m, 2H), 3.78 (s, 3H), 3.10 (s, 3H), 2.45 (s, 3H). MS (M+H)⁺: 469.

### Examples 8-12

Examples 8-12 were prepared according to the general procedures of Example 7.

| Ex- | | | ¹H-NMR (400 MHz, | MS |
|---|---|---|---|---|
| ample | Structure | Name | CDCl₃) δ | (M + H)⁺ |
| 8 | 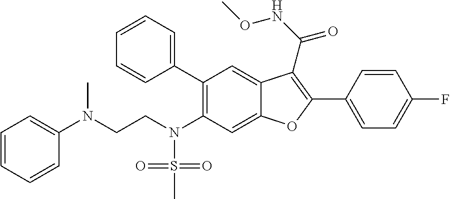 | 2-(4-fluorophenyl)- N-methoxy-6-[{2- [methyl(phenyl) amino]ethyl} (methylsulfonyl) amino]-5-phenyl-1- benzofuran-3- carboxamide | 8.40 (s, 1H), 7.87~7.91 (m, 2H), 7.71 (s, 1H), 7.50 (s, 1H), 7.32~7.40 (m, 5H), 7.23~7.25 (m, 2H), 7.12~7.19 (m, 2H), 6.93~6.95 (m, 1H), 6.82 (d, J = 8.0 Hz, 2H), 3.80 (s, 3H), 3.31~3.45 (m, 4H), 2.80 (d, J = 12.0 Hz, 3H), 2.74 (s, 3H). | 588 |
| 9 | 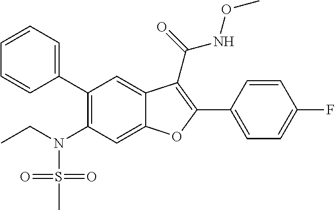 | 6-[ethyl (methylsulfonyl) amino]-2-(4- fluorophenyl)-N- methoxy-5-phenyl- 1-benzofuran-3- carboxamide | 8.35 (s, 1H), 7.87~7.90 (m, 2H), 7.68 (s, 1H), 7.51 (s, 1H), 7.32~7.41 (m, 5H), 7.14 (t, J = 8.8 Hz, 2H), 3.79 (s, 3H), 3.23~3.50 (m, 2H), 2.65 (s, 3H), 1.01 (t, J = 7.2 Hz, 3H). | 483 |
| 10 | 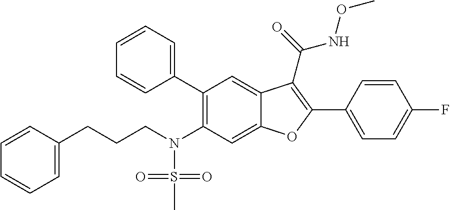 | 2-(4-fluorophenyl)- N-methoxy-6- [(methylsulfonyl) (3-phenylpropyl) amino]-5-phenyl-1- benzofuran-3- carboxamide | 8.35 (s, 1H), 7.86~7.89 (m, 2H), 7.64 (s, 1H), 7.48 (s, 1H), 7.31~7.35 (m, 5H), 7.09~7.19 (m, 5H), 6.94 (d, J = 7.2 Hz, 2H), 3.37 (s, 3H), 3.09~3.41 (m, 2H), 2.67 (s, 3H), 2.34 (d, J = 3.6 Hz, 2H), 1.68 (m, 2H). | 573 |
| 11 | 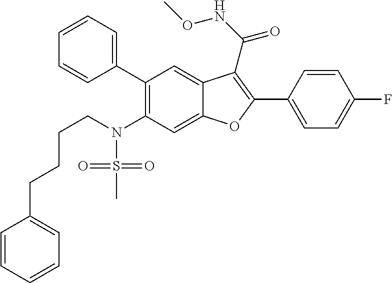 | 2-(4-fluorophenyl)- N-methoxy-6- [(methylsulfonyl) (4-phenylbutyl) amino]-5-phenyl-1- benzofuran-3- carboxamide | 7.93~7.97 (s, 2H), 7.72 (s, 1H), 7.54 (s, 1H), 7.14~7.45 (m, 10H), 7.04 (d, J = 7.2 Hz, 2H), 3.85 (s, 3H), 3.12~3.40 (m, 2H), 2.73 (s, 3H), 2.47 (s, 2H), 1.36~1.45 (s, 4H). | 587 |
| 12 | 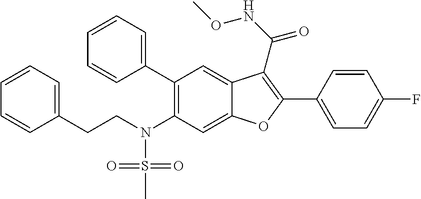 | 2-(4-fluorophenyl)- N-methoxy-6- [(methylsulfonyl) (2-phenylethyl) amino]-5-phenyl-1- benzofuran-3- carboxamide | 8.28 (s, 1H), 7.93~7.91 (m, 2H), 7.73 (s, 1H), 7.48~7.43 (m, 2H), 7.40~7.33 (m, 3H), 7.121~7.11 (m, 4H), 7.00~6.98 (m, 2H), 3.80 (s, 3H), 3.63~3.27 (m, 2H), 2.68~2.64 (m, 2H), 2.56 (s, 3H). | 559 |

### Example 13

### 6-[(cyclohexylmethyl)(methylsulfonyl)amino]-2-(4-fluorophenyl)-N-methyl-5-phenyl-1-benzofuran-3-carboxamide

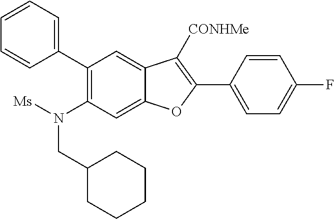

### Steps 1-3

Steps 1-3 were performed in accordance with Example 1, Steps 1-3.

### Step 4: 2-(4-fluorophenyl)-6-[(methylsulfonyl)amino]-5-phenyl-1-benzofuran-3-carboxylic acid

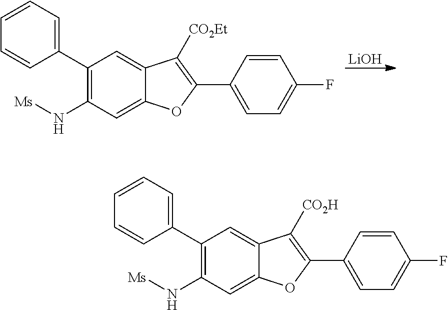

The compound prepared in Step 3 (1.3 g, 2.74 mmol) was dissolved in 1,4-dioxane (7 mL) and H₂O (7 mL). Then, LiOH (1.14, 27.4 mmol) was added to the solution, and the mixture was refluxed for 2 hours. After acidified with HCl (1 N) and extracted with EtOAc, the combined organic phases were washed with brine, dried over Na₂SO₄, filtered and evaporated to give the carboxylic acid (990 mg, yield: 85%). It was used for the next step without further purification.

### Step 5: 2-(4-fluorophenyl)-N-methyl-6-[7-methylsulfonyl)amino]-5-phenyl-1-benzofuran-3-carboxamide

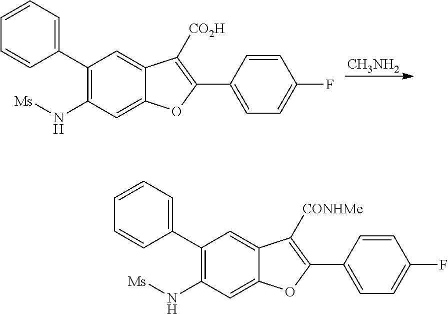

The carboxylic acid prepared in Step 4 (990 mg, 2.34 mmol), HOBT (631 mg, 4.7 mmol) and EDCI (900 mg, 4.7 mmol) were dissolved in dry DMF (10 mL). The resulting solution was stirred for 30 minutes. Then, methanamine (HCl salt, 640 mg, 9.4 mmol) and Et₃N (2 mL) were added to the mixture. After stirred overnight, the mixture was diluted with water and extracted with EtOAc. The combined organic phases were washed with brine, dried over Na₂SO₄, filtered and evaporated. The crude product was purified by column to give pure 2-(4-fluorophenyl)-N-methyl-6-[(methylsulfonyl)amino]-5-phenyl-1-benzofuran-3-carboxamide (900 mg, yield: 88%).

¹H-NMR (400 MHz, CDCl₃) δ 7.82˜7.86 (m, 2H), 7.79 (s, 1H), 7.63 (s, 1H), 7.41˜7.46 (m, 3H), 7.27˜7.33 (m, 21˜1), 7.10˜7.44 (m, 2H), 6.51 (br, 1H), 5.84 (br, 1H), 2.91 (d, J=4.8 Hz, 3H), 2.80 (s, 3H). MS (M+H)⁺: 439.

### Step 6: 6-[(cyclohexylmethyl)(methylsulfonyl)amino]-2-(4-fluorophenyl)-N-methyl-5-phenyl-1-benzofuran-3-carboxamide

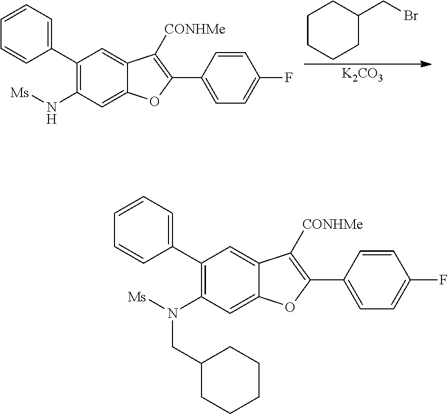

The compound prepared in Step 5 (35 mg, 0.08 mmol), (bromomethyl)cyclohexane (21 mg, 0.12 mmol), K₂CO₃ (22 mg, 0.16 mmol), KI (2 mg) in DMF (2 mL) was stirred at 90° C. for 16 hours under N₂. The mixture was concentrated, diluted with DCM, washed with brine, dried over Na₂SO₄, filtered and the solvent was evaporated. The residue was purified by prep-HPLC to give pure product (15 mg, yield: 35%).

¹H-NMR (400 MHz, CDCl₃) δ 7.97˜7.93 (m, 2H), 7.73 (s, 1H), 7.58 (s, 1H), 7.52˜7.50 (m, 2H), 7.44˜7.37 (m, 3H), 7.24˜7.16 (m, 2H), 5.84 (s, 1H), 3.18˜3.13 (m, 1H), 2.99˜2.97 (m, 4H), 2.95 (s, 3H), 1.74˜1.58 (m, 1H), 1.54˜1.51 (m, 2H), 1.43˜1.41 (m, 2H), 1.04˜0.91 (m, 4H), 0.89˜0.79 (m, 2H), 0.76˜0.56 (m, 1H). MS (M+H)⁺: 535.

### Examples 14-68

Examples 14-68 were prepared according to the general procedures of Example 13.

| Ex- | | | ¹H-NMR (400 MHz, | MS |
|---|---|---|---|---|
| ample | Structure | Name | CDCl₃) δ | (M + H)⁺ |
| 14 | 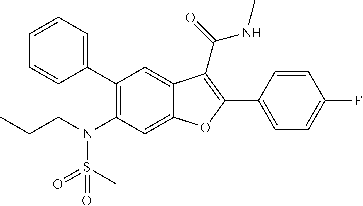 | 2-(4-fluorophenyl)- N-methyl-6- [(methylsulfonyl) (propyl)amino]-5- phenyl-1-benzofuran- 3-carboxamide | 7.85~7.89 (m, 2H), 7.66 (s, 1H), 7.48 (s, 1H), 7.31~7.42 (m, 5H), 7.12 (t, J = 8.4 Hz, 2H), 5.89 (d, J = 3.6 Hz, 1H), 3.06~3.11 (m, 2H), 2.89 (d, J = 5.2 Hz, 3H), 2.70 (s, 3H), 1.36 (d, J = 4.4 Hz, 2H), 0.67 (t, J = 7.2 Hz, 3H). | 481 |
| 15 | 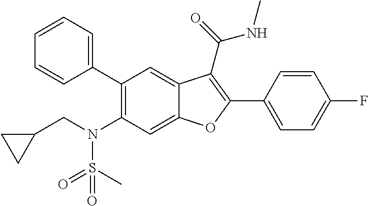 | 6- [(cyclopropylmethyl) (methylsulfonyl) amino]-2-(4- fluorophenyl)-N- methyl-5-phenyl-1- benzofuran-3- carboxamide | 7.95~7.99 (m, 2H), 7.76 (s, 1H), 7.65 (s, 1H), 7.50~7.52 (m, 2H), 7.40~7.43 (m, 3H), 7.18~7.26 (m, 2H), 5.88 (br s, 1H), 3.38~3.42 (m, 1H), 3.02~3.04 (m, 1H), 2.99 (d, J = 4.8 Hz, 3H), 0.88~0.92 (m, 1H), 0.44 (m, 2H), 0.11 (br s, 1H), 0.01 (br s, 1H), 0.65 (t, J = 7.2 Hz, 3H). | 493 |
| 16 | 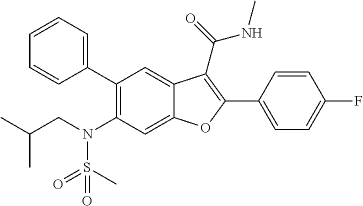 | 2-(4-fluorophenyl)- N-methyl-6-[(2- methylpropyl) (methylsulfonyl) amino]-5-phenyl-1- benzofuran-3- carboxamide | 7.89 (t, J = 4.8 Hz, 2H), 7.66 (s, 1H), 7.53 (s, 1H), 7.32~7.45 (m, 5H), 7.12 (t, J = 8.8 Hz, 2H), 5.82 (s, 1H), 3.06~3.11 (m, 1H), 2.80~2.91 (m, 7H), 1.33~1.40 (m, 1H), 0.73 (d, J = 6.4 Hz, 3H), 0.43 (d, J = 6.4 Hz, 3H). | 495 |
| 17 | 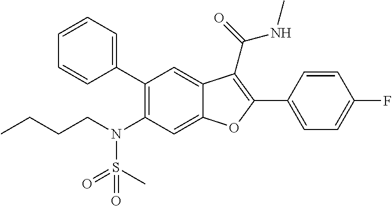 | 6-[butyl (methylsulfonyl) amino]-2-(4- fluorophenyl)-N- methyl-5-phenyl-1- benzofuran-3- carboxamide | 7.97~7.93 (m, 2H), 7.73 (s, 1H), 7.58 (s, 1H), 7.52~7.50 (m, 2H), 7.44~7.37 (m, 3H), 7.24~7.16 (m, 2H), 5.90 (s, 1H), 3.41~3.33 (m, 1H), 3.21~3.10 (m, 1H), 3.09~3.08 (d, J = 0.4 Hz, 3H), 2.84 (s, 3H), 1.38~1.37 (m, 2H), 1.24~1.12 (m, 2H), 0.80~0.76 (t, J = 1.6 Hz, 3H). | 495 |
| 18 | 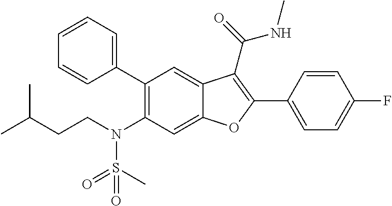 | 2-(4-fluorophenyl)- N-methyl-6-[(3- methylbutyl) (methylsulfonyl) amino]-5-phenyl-1- benzofuran-3- carboxamide | 7.90~7.94 (m, 2H), 7.72 (s, 1H), 7.53 (s, 1H), 7.37~7.47 (m, 5H), 7.17 (t, J = 8.4 Hz, 2H), 5.95 (d, J = 4.4 Hz, 1H), 3.13~3.69 (m, 4H), 2.96 (d, J = 4.8 Hz, 3H), 2.76 (s, 3H), 1.34~1.39 (m, 1H), 0.76 (d, J = 5.2 Hz, 6H). | 509 |
| 19 | 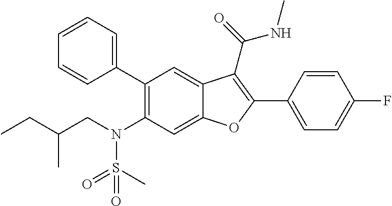 | 2-(4-fluorophenyl)- N-methyl-6-[(2- methylbutyl) (methylsulfonyl) amino]-5-phenyl-1- benzofuran-3- carboxamide | 7.67~7.92 (m, 2H), 7.53 (s, 1H), 7.46 (s, 1H), 7.23~7.43 (m, 5H), 7.11~7.16 (m, 2H), 5.78 (br, 1H), 2.93~3.02 (m, 2H), 2.91 (s, 3H), 2.86 (d, J = 7.2 Hz, 3H), 1.11~1.251 (m, 2H), 0.94~0.97 (m, 1H), 0.73 (d, J = 6.8 Hz, 3H), 0.59 (d, J = 7.2 Hz, 3H). | 509 |
| 20 | 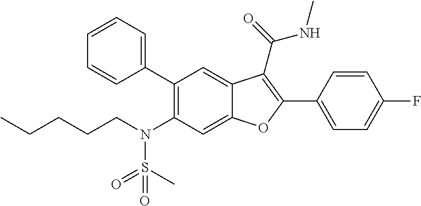 | 2-(4-fluorophenyl)- N-methyl-6- [(methylsulfonyl) (pentyl)amino]-5- phenyl-1-benzofuran- 3-carboxamide | 7.89~7.92 (m, 2H), 7.70 (s, 1H), 7.51 (s, 1H), 7.34~7.43 (m, 5H), 7.11~7.16 (m, 2H), 5.78 (br, 1H), 3.03~3.43 (m, 2H), 2.92 (d, J = 5.2 Hz, 3H), 2.72 (s, 3H), 0.97~1.38 (m, 6H), 0.73~0.75 (d, J = 7.2 Hz, 3H). | 509 |
| 21 | 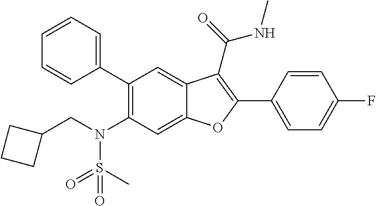 | 6- [(cyclobutylmethyl) (methylsulfonyl) amino]-2-(4- fluorophenyl)-N- methyl-5-phenyl-1- benzofuran-3- carboxamide | 7.88~7.92 (m, 2H), 7.68 (s, 1H), 7.47 (s, 1H), 7.34~7.45 (m, 5H), 7.11~7.16 (m, 2H), 5.78 (br, 1H), 3.07~3.43 (m, 2H), 2.92 (d, J = 5.2 Hz, 3H), 2.78 (m, 3H), 2.21~2.28 (m, 1H), 1.33~1.89 (m, 6H). | 507 |
| 22 | 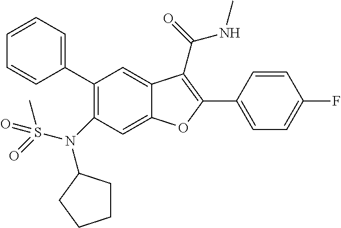 | 6-[cyclopentyl (methylsulfonyl) amino]-2-(4- fluorophenyl)-N- methyl-5-phenyl-1- benzofuran-3- carboxamide | 7.92~7.95 (dd, J = 4.0 Hz, 2H), 7.75 (s, 1H), 7.41~7.44 (m, 3H), 7.37~7.39 (m, 2H), 7.22 (s, 1H), 7.17~7.20 (m, 2H), 5.95 (s, 1H), 3.96~4.02 (m, 1H), 2.98~3.00 (d, J= 8.0 Hz, 3H), 2.79 (s, 3H), 1.91~1.96 (m, 1H), 1.39~1.56 (m, 6H), 1.11~1.16 (m, 1H). | 507 |
| 23 | 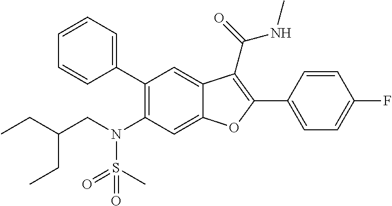 | 6-[(2-ethylbutyl) (methylsulfonyl) amino]-2-(4- fluorophenyl)-N- methyl-5-phenyl-1- benzofuran-3- carboxamide | 7.89~7.86 (m, 2H), 7.64 (s, 1H), 7.52 (s, 1H), 7.47~7.44 (m, 2H), 7.34~7.32 (m, 3H), 7.15~7.10 (m, 2H), 5.93 (s, 1H), 4.50~4.41 (m, 1H), 3.20~3.19 (m, 1H), 2.96~2.94 (d, J = 0.8 Hz, 3H), 2.88 (s, 3H), 1.21~1.16 (m, 2H), 1.13~1.04 (m, 1H), 1.00~0.92 (m, 2H), 0.67~0.64 (t, J = 1.2 Hz, 3H), 0.53~0.50 (t, J = 1.2 Hz, 3H). | 523 |
| 24 | 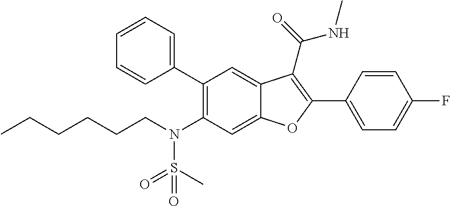 | 2-(4-fluorophenyl)-6- [hexyl (methylsulfonyl) amino]-N-methyl-5- phenyl-1- benzofuran-3- carboxamide | 7.91~7.90 (m, 2H), 7.73 (s, 1H), 7.58 (s, 1H), 7.52~7.50 (m, 2H), 7.44~7.37 (m, 3H), 7.24~7.16 (m, 2H), 6.06 (s, 1H), 3.39~3.38 (m, 1H), 3.15~3.14 (m, 1H), 2.98~2.97 (d, J = 0.4 Hz, 3H), 2.76 (s, 3H), 1.38~1.35 (m, 2H), 1.18~1.12 (m, 6H), 0.82~0.78 (t, J = 1.6 Hz, 3H). | 523 |
| 25 | 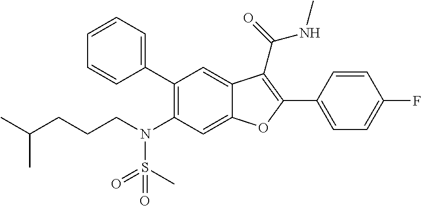 | 2-(4-fluorophenyl)- N-methyl-6-[(4- methylpentyl) (methylsulfonyl) amino]-5-phenyl-1- benzofuran-3- carboxamide | 7.91~7.90 (m, 2H), 7.73 (s, 1H), 7.58 (s, 1H), 7.52~7.50 (m, 2H), 7.44~7.37 (m, 3H), 7.24~7.16 (m, 2H), 5.85 (s, 1H), 3.39~3.38 (m, 1H), 3.17~3.14 (m, 1H), 2.98~2.97 (d, J = 0.4 Hz, 3H), 2.78 (s, 3H), 1.42~1.33 (m, 3H), 0.98~0.98 (m, 2H), 0.77~0.75 (t, J = 0.8 Hz, 6H). | 523 |
| 26 | 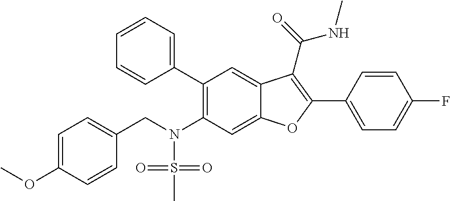 | 2-(4-fluorophenyl)-6- [(4-methoxybenzyl) (methylsulfonyl) amino]-N-methyl-5- phenyl-1-benzofuran- 3-carboxamide | 7.85~7.89 (m, 2H), 7.64 (s, 1H), 7.30~7.35 (m, 5H), 7.26 (m, 1H), 7.09~7.14 (m, 2H), 6.88~6.90 (m, 2H), 6.66~6.68 (m, 2H), 5.87 (br s, 1H), 4.09~4.48 (br ABq, 2H), 3.70 (s, 3H), 2.89 (d, J = 4.8 Hz, 3H), 2.63 (s, 3H). | 559 |
| 27 | 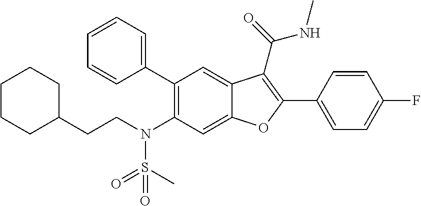 | 6-[(2- cyclohexylethyl) (methylsulfonyl) amino]-2-(4- fluorophenyl)-N- methyl-5-phenyl-1- benzofuran-3- carboxamide | 7.91~7.90 (m, 2H), 7.73 (s, 1H), 7.58 (s, 1H), 7.44~7.37 (m, 5H), 7.24~7.16 (m, 2H), 5.77 (s, 1H), 3.42~3.40 (m, 1H), 3.38~3.36 (m, 1H), 3.15~3.12 (d, J = 1.2 Hz, 3H), 2.71 (s, 3H), 1.56~1.50 (m, 5H), 1.24~1.23 (m, 2H), 1.11~1.01 (m, 4H), 0.95~0.72 (m, 2H). | 549 |
| 28 | 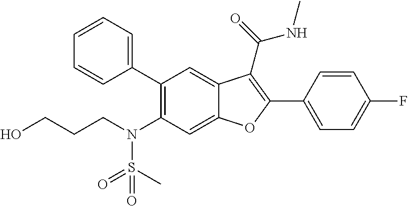 | 2-(4-fluorophenyl)-6- [(3-hydroxypropyl) (methylsulfonyl) amino]-N-methyl-5- phenyl-1-benzofuran- 3-carboxamide | 7.97~7.93 (m, 2H), 7.73 (s, 1H), 7.58 (s, 1H), 7.52~7.50 (m, 2H), 7.44~7.37 (m, 3H), 7.24~7.16 (m, 2H), 5.81 (s, 1H), 3.44~3.22 (m, 4H), 2.93~2.92 (d, J = 0.4 Hz, 3H), 2.79 (s, 3H), 1.19~1.17 (m, 2H). | 497 |
| 29 | 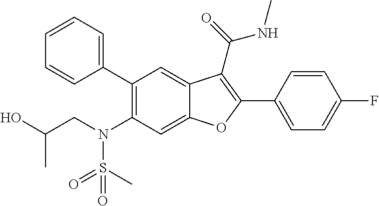 | 2-(4-fluorophenyl)-6- [(2-hydroxypropyl) (methylsulfonyl) amino]-N-methyl-5- phenyl-1-benzofuran- 3-carboxamidc | 7.91~7.90 (m, 2H), 7.73 (s, 1H), 7.58 (s, 1H), 7.44~7.37 (m, 5H), 7.24~7.16 (m, 2H), 5.82 (s, 1H), 3.71~3.68 (m, 1H), 3.43~3.38 (m, 3H), 2.98~2.92 (m, 2H), 2.76 (s, 3H), 0.93 (m, 3H). | 497 |
| 30 | 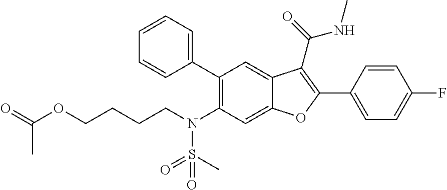 | 4-{[2-(4- fluorophenyl)-3- (methylcarbamoyl)- 5-phenyl-1- benzofuran-6-yl] (methylsulfonyl) amino}butyl acetate | 7.88~7.92 (m, 2H), 7.71 (s, 1H), 7.52 (s, 1H), 7.33~7.44 (m, 5H), 7.12~7.16 (m, 2H), 5.78 (br, 1H), 3.82~3.85 (m, 2H), 3.07~3.50 (m, 2H), 2.92 (d, J = 4.8 Hz, 3H), 2.76 (m, 3H), 1.95 (s, 3H), 1.28~1.57 (m, 4H). | 553 |
| 31 | 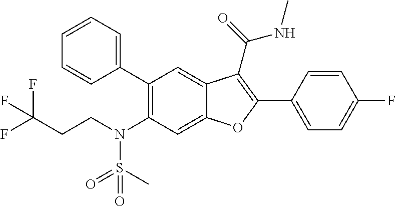 | 2-(4-fluorophenyl)- N-methyl-6- [(methylsulfonyl) (3,3,3- trifluoropropyl) amino]-5-phenyl-1- benzofuran-3- carboxamide | 7.91~7.90 (m, 2H), 7.73 (s, 1H), 7.58 (s, 1H), 7.44~7.37 (m, 5H), 7.24~7.16 (m, 2H), 5.99 (s, 1H), 3.61~3.38 (m, 2H), 2.98 (s, 3H), 2.82 (s, 3H), 2.24~2.17 (m, 2H). | 535 |
| 32 | 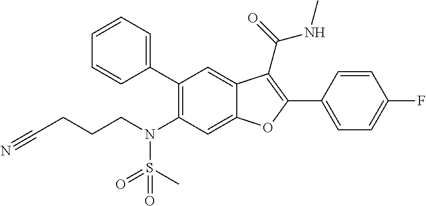 | 6-[(3-cyanopropyl) (methylsulfonyl) amino]-2-(4- fluorophenyl)-N- methyl-5-phenyl-1- benzofuran-3- carboxamide | 7.87~7.90 (m, 2H), 7.74 (s, 1H), 7.52 (s, 1H), 7.35~7.47 (m, 5H), 7.15 (t, J = 8.4 Hz, 2H), 5.79 (d, J = 3.6 Hz, 1H), 3.40 (t, J = 6.4 Hz, 2H), 2.91 (d, J = 5.2 Hz, 3H), 2.87 (s, 3H), 1.83 (d, J = 6.0 Hz, 2H), 1.59 (s, 2H). | 506 |
| 33 | 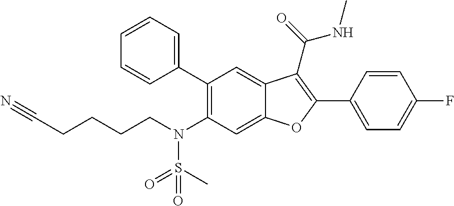 | 6-[(4-cyanobutyl) (methylsulfonyl) amino]-2-(4- fluorophenyl)-N- methyl-5-phenyl-1- benzofuran-3- carboxamide | 7.91~7.95 (m, 2H), 7.77 (s, 1H), 7.56 (s, 1H), 7.39~7.50 (m, 5H), 7.20 (t, J = 8.4 Hz, 2H), 5.27 (d, J = 4.4 Hz, 1H), 3.29~3.38 (m, 2H), 2.96 (d, J = 4.8 Hz, 3H), 2.86 (s, 3H), 2.15 (d, J = 7.6 Hz, 2H), 1.48 (s, 2H), 1.36 (d, J = 4.8 Hz, 2H). | 520 |
| 34 | 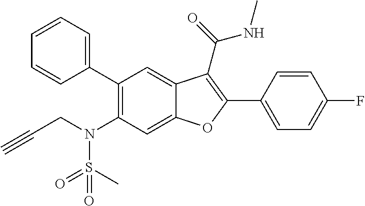 | 2-(4-fluorophenyl)- N-methyl-6- [(methylsulfonyl) (prop-2-yn-1- yl)amino]-5-phenyl- 1-benzofuran-3- carboxamide | 7.97~7.93 (m, 2H), 7.73 (s, 1H), 7.58 (s, 1H), 7.52~7.50 (m, 2H), 7.44~7.37 (m, 3H), 7.24~7.16 (m, 2H), 5.96 (s, 1H), 4.36~4.28 (m, 1H), 3.94~3.77 (m, 1H), 2.98~2.96 (m, 6H), 2.38 (s, 1H). | 477 |
| 35 | 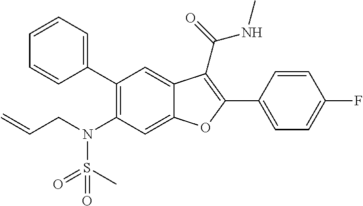 | 2-(4-fluorophenyl)- N-methyl-6- [(methylsulfonyl) (prop-2-en-1- yl)amino]-5-phenyl- 1-benzofuran-3- carboxamide | 7.85~7.89 (m, 2H) 7.68 (s, 1H), 7.36~7.46 (m, 6H), 7.11~7.19 (m, 2H), 5.86 (d, J = 4.0 Hz, 1H), 5.70~5.77 (m, 1H), 5.00~5.08 (m, 2H), 3.70~4.02 (m, 2H), 2.91 (d, J = 4.8 Hz, 3H), 2.67 (s, 3H). | 479 |
| 36 | 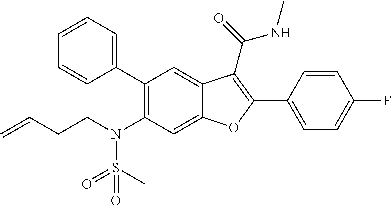 | 6-[but-3-en-1-yl (methylsulfonyl) amino]-2-(4- fluorophenyl)-N- methyl-5-phenyl-1- benzofuran-3- carboxamide | 7.92~7.96 (m, 2H), 7.75 (s, 1H), 7.55 (s, 1H), 7.44~7.50 (m, 2H), 7.39~7.42 (m, 3H), 7.16~7.21 (m, 2H), 5.97 (br s, 1H), 5.51~5.62 (m, 1H), 4.96~5.01 (m, 2H), 3.49 (br s, 1H), 3.17 (br s, 1H), 2.96 (d, J = 4.8 Hz, 3H), 2.81 (s, 3H), 2.15~2.17 (m, 2H). | 493 |
| 37 | 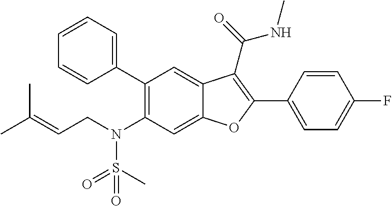 | 2-(4-fluorophenyl)- N-methyl-6-[(3- methylbut-2-en-1- yl)(methylsulfonyl) amino]-5-phenyl-1- benzofuran-3- carboxamide | 7.91~7.95 (m, 2H), 7.77 (s, 1H), 7.56 (s, 1H), 7.39~7.50 (m, 5H), 7.17 (t, J = 8.4 Hz, 2H), 5.94 (s, 1H), 5.11 (t, J = 3.2 Hz, 1H), 3.76~4.02 (m, 2H), 2.95 (d, J = 4.8 Hz, 3H), 2.75 (s, 3H), 1.65 (s, 3H), 1.29 (s, 3H). | 507 |
| 38 | 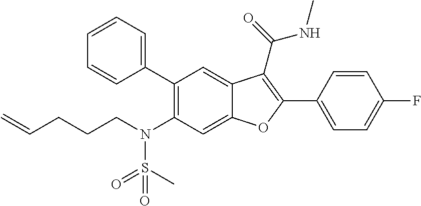 | 2-(4-fluorophenyl)- N-methyl-6- [(methylsulfonyl) (pent-4-en-1- yl)amino]-5-phenyl- 1-benzofuran-3- carboxamide | 7.93~7.96 (m, 2H), 7.74 (s, 1H), 7.56 (s, 1H), 7.37~7.47 (m, 5H), 7.16~7.24 (m, 2H), 5.82 (s, 1H), 5.56~5.65 (m, 1H), 4.86~4.91 (m, 2H) 3.18~3.47 (m, 2H), 3.10 (d, J = 8.4 Hz, 3H), 2.77 (s, 3H). 1.82~1.85 (m, 2H), 1.50 (m, 2H). | 507 |
| 39 | 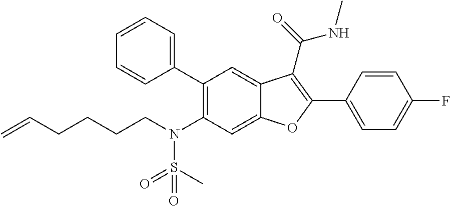 | 2-(4-fluorophenyl)-6- (hex-5-en-1- yl(methylsulfonyl) amino]-N-methyl-5- phenyl-1-benzofuran- 3-carboxamide | 7.93~7.97 (m, 2H), 7.74 (s, 1H), 7.55 (s, 1H), 7.37~7.44 (m, 5H), 7.16~7.24 (m, 2H), 5.81 (s, 1H), 5.61~5.68 (m, 1H), 4.87~4.92 (m, 2H), 3.38~3.47 (m, 2H), 3.17 (d, J = 12 Hz, 3H), 2.77 (s, 3H), 1.88~1.92 (m, 2H), 1.39 (m, 2H), 1.18 (m, 2H). | 521 |
| 40 | 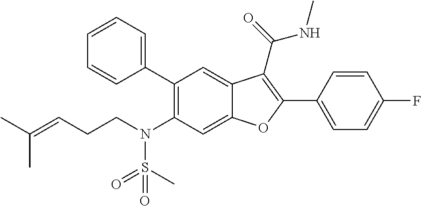 | 2-(4-fluorophenyl)- N-methyl-6-[(4- methylpent-3-en-1- yl)(methylsulfonyl) amino]-5-phenyl-1- benzofuran-3- carboxamide | 7.86~7.90 (m, 2H), 7.68 (s, 1H), 7.34~7.49 (m, 6H), 7.11~7.16 (m, 2H), 5.88 (s, 1H), 4.82 (s, 1H), 3.34~3.47 (m, 2H), 2.73~2.93 (m, 6H), 2.00~2.04 (m, 2H), 1.56 (s, 3H), 1.41 (s, 3H). | 521 |
| 41 | 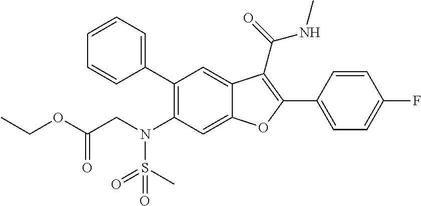 | ethyl N-[2-(4- fluorophenyl)-3- (methylcarbamoyl)- 5-phenyl-1- benzofuran-6-yl]-N- (methylsulfonyl) glycinate | 7.86~7.90 (dd, J = 8.0 Hz, 2H), 7.82 (s, 1H), 7.69 (s, 1H), 7.48~7.50 (m, 2H), 7.34~7.38 (m, 3H), 7.11~7.15 (m, 2H), 5.86 (s, 1H), 4.90~4.24 (m, 2H), 4.03~4.08 (dd, J = 8.0 Hz, 2H), 3.18 (s, 3H), 2.91~2.93 (d, J = 8.0 Hz, 3H), 1.12~1.16 (m, 3H). | 525 |
| 42 | 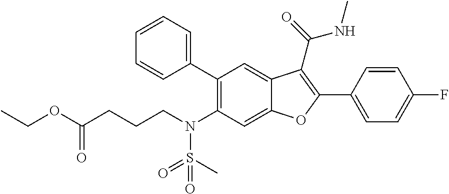 | ethyl 4-{[2-(4- fluorophenyl)-3- (methylcarbamoyl)- 5-phenyl-1- benzofuran-6- yl](methylsulfonyl) amino}butanoate | 7.97~7.93 (m, 2H), 7.73 (s, 1H), 7.58 (s, 1H), 7.52~7.50 (m, 2H), 7.44~7.37 (m, 3H), 7.24~7.16 (m, 2H), 5.76 (s, 1H), 4.02~3.99 (q, J = 1.2 Hz, 2H), 3.45~3.37 (m, 1H), 3.22~3.19 (m, 1H), 2.93~2.91 (d, J = 0.8 Hz, 3H), 2.77 (s, 3H), 2.00~1.95 (m, 2H), 1.69~1.63 (m, 2H), 1.16~1.13 (t, J = 1.2 Hz, 3H). | 553 |
| 43 | 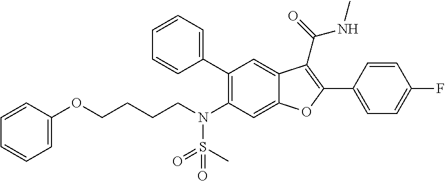 | 2-(4-fluorophenyl)- N-methyl-6- [(methylsulfonyl)(4- phenoxybutyl)amino]- 5-phenyl-1- benzofuran-3- carboxamide | 7.93~7.97 (m, 2H), 7.74 (s, 1H), 7.57 (s, 1H), 7.46~7.48 (m, 2H), 7.36~7.42 (m, 3H), 7.16~7.23 (m, 4H), 6.88~6.92 (m, 1H), 6.79 (d, J = 8.0 Hz, 2H), 5.81 (s, 1H), 3.79 (s, 2H), 3.47~3.54 (m, 1H), 3.22~3.30 (m, 1H), 2.97 (d, J = 4.0 Hz, 3H), 2.80 (s, 3H), 1.53 (m, 4H). | 587 |
| 44 | 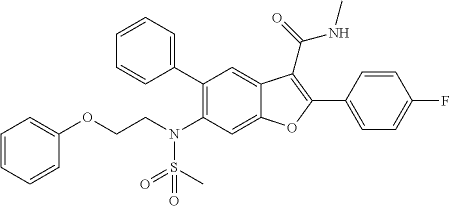 | 2-(4-fluorophenyl)- N-methyl-6- [(methylsulfonyl)(2- phenoxyethyl)amino]- 5-phenyl-1- benzofuran-3- carboxamide | 7.91~7.94 (m, 2H), 7.77 (s, 1H), 7.51~7.54 (m, 3H), 7.36~7.42 (m, 3H), 7.34 (s, 1H), 7.22 (s, 1H), 7.15~7.19 (m, 2H), 6.93~6.96 (m, 1H), 6.75 (d, J = 8.0 Hz, 2H), 5.81 (s, 1H), 3.96 (s, 1H), 3.82 (s, 2H), 3.26 (s, 1H), 3.09 (s, 3H), 2.97 (d, J = 4.0 Hz, 3H). | 559 |
| 45 | 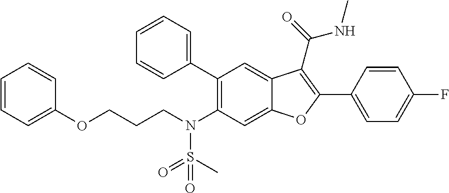 | 2-(4-fluorophenyl)- N-methyl-6- [(methylsulfonyl)(3- phenoxypropyl) amino]-5-phenyl-1- benzofuran-3- carboxamide | 7.93~7.96 (m, 2H), 7.77 (s, 1H), 7.63 (s, 1H), 7.49~7.51 (m, 2H), 7.39~7.44 (m, 3H), 7.16~7.19 (m, 4H), 6.88~6.91 (m, 1H), 6.69 (d, J = 8.0 Hz, 2H), 5.80 (s, 1H), 3.54~3.57 (m, 3H), 3.43 (s, 1H), 2.98 (d, J = 8.0 Hz, 3H), 2.79 (s, 3H), 1.84 (s, 1H), 1.81 (s, 3H). | 573 |
| 46 | 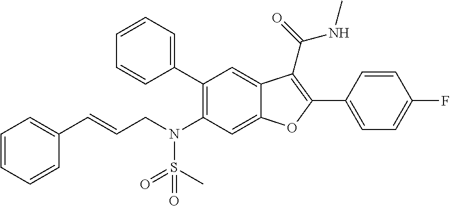 | 2-(4-fluorophenyl)- N-methyl-6- {(methylsulfonyl)[(2 E)-3-phenylprop-2- en-1-yl]amino}-5- phenyl-1-benzofuran- 3-carboxamide | 7.86~7.91 (m, 2H), 7.69 (s, 1H), 7.49 (s, 1H), 7.32~7.42 (m, 5H), 7.22~7.24 (m, 4H), 7.09~7.20 (m, 2H), 6.29 (d, J = 16 Hz, 1H), 5.96~6.04 (m, 1H), 5.73 (s, 1H), 4.13 (s, 1H), 4.15 (s, 1H), 2.91 (d, J = 4.0 Hz, 3H), 2.72 (s, 3H). | 555 |
| 47 | 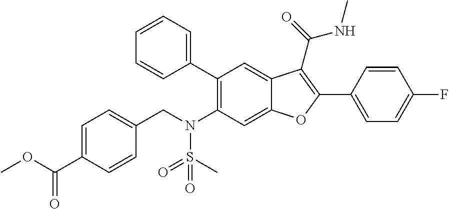 | methyl 4-({[2-(4- fluorophenyl)-3- (methylcarbamoyl)- 5-phenyl-1- benzofuran-6- yl](methylsulfonyl) amino}methyl) benzoate | 7.85~7.89 (m, 2H), 7.78~7.82 (m, 2H), 7.66 (s, 1H), 7.72~7.35 (m, 6H), 7.10~7.14 (m, 2H), 7.05 (d, J = 8.0 Hz, 2H), 5.74 (m, 1H), 4.55 (s, 1H), 4.23 (s, 1H), 3.83 (s, 3H), 2.91 (d, J = 4.0 Hz, 3H), 2.70 (s, 3H). | 587 |
| 48 | 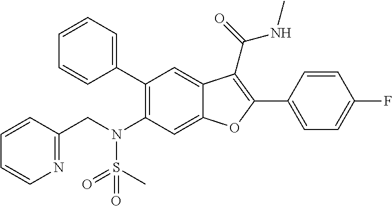 | 2-(4-fluorophenyl)- N-methyl-6- [(methylsulfonyl) (pyridin-2- ylmethyl)amino]-5- phenyl-1-benzofuran- 3-carboxamide | 8.49 (d, J = 4.0 Hz, 1H), 7.85~7.89 (m, 2H), 7.68~7.72 (m, 1H), 7.62 (m, 1H), 7.30~7.39 (m, 7H), 7.21 (s, 1H), 7.09~7.14 (m, 2H), 5.84 (m, 1H), 4.73 (s, 2H), 3.05 (s, 3H), 2.91 (d, J = 8.0 Hz, 3H). | 530 |
| 49 | 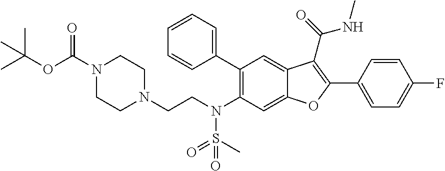 | tert-butyl 4-(2-{[2- (4-fluorophenyl)-3- (methylcarbamoyl)- 5-phenyl-1- benzofuran-6- yl](methylsulfonyl) amino}ethyl) piperazine-1- carboxylate | 7.93 (m, 2H), 7.73 (s, 1H), 7.51~7.54 (m, 3H), 7.36~7.41 (m, 3H), 7.14~7.18 (m, 2H) 6.08 (br s, 1H), 3.67 (br s, 1H), 3.36 (s, 4H), 3.04 (s, 4H), 2.93 (d, J = 4.8 Hz, 3H), 2.36 (br s, 3H), 2.22 (br s, 3H), 1.42 (s, 9H). | 651 |
| 50 | 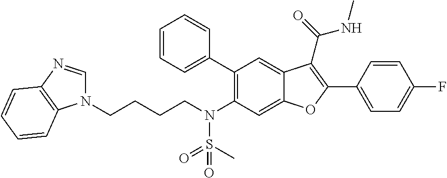 | 6-{[4-(1H- benzimidazol-1- yl)butyl] (methylsulfonyl) amino}-2-(4- fluorophenyl)-N- methyl-5-phenyl-1- benzofuran-3- carboxamide | 9.12 (s, 1H), 7.89~7.81 (m, 3H), 7.67 (s, 1H), 7.51~7.41 (m, 3H), 7.32~7.28 (m, 3H), 7.25~7.21 (m, 2H), 7.21 (s, 1H), 7.15~7.11 (m, 2H), 6.08 (s, 1H), 4.52~4.42 (m, 1H), 4.20~4.12 (m, 2H), 3.97~3.91 (m, 1H), 3.33~3.31 (m, 2H), 2.92 (s, 3H), 2.85 (s, 3H), 1.34~1.31 (m, 2H). | 611 |
| 51 | 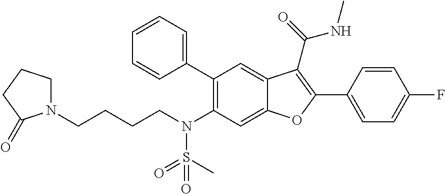 | 2-(4-fluorophenyl)- N-methyl-6- {(methylsulfonyl)[4- (2-oxopyrrolidin-1- yl)butyl]amino}-5- phenyl-1-benzofuran- 3-carboxamide | 7.88~7.92 (m, 2H), 7.69 (s, 1H), 7.50 (s, 1H), 7.41~7.43 (m, 2H), 7.31~7.38 (m, 3H), 7.11~7.15 (m, 2H), 5.84 (s, 1H), 3.08~3.36 (m, 6H), 2.92 (d, J = 8.0 Hz, 3H), 2.76 (s, 3H), 2.30 (t, J = 8.0 Hz, 2H), 1.18~1.19 (m, 2H), 1.27 (m, 4H). | 578 |
| 52 | 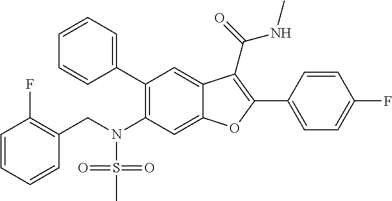 | 6-[(2- fluorobenzyl)(methyl sulfonyl)amino]-2-(4- fluorophenyl)-N- methyl-5-phenyl-1- benzofuran-3- carboxamide | 7.91~7.90 (m, 2H), 7.73 (s, 1H), 7.44~7.37 (m, 6H), 7.17~6.96 (m, 3H), 6.95~6.91 (m, 1H), 6.90~6.73 (m, 2H), 5.81 (s, 1H), 4.90~4.24 (m, 2H), 2.96 (s, 3H), 2.76 (s, 3H). | 547 |
| 53 | 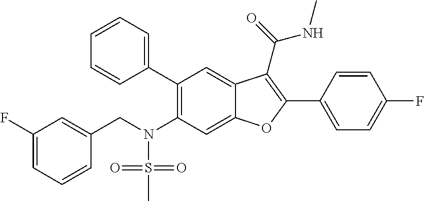 | 6-[(3- fluorobenzyl)(methyl- sulfonyl)amino]-2-(4- fluorophenyl)-N- methyl-5-phenyl-1- benzofuran-3- carboxamide | 7.86~7.88 (m, 2H), 7.59 (s, 1H), 7.36 (s, 1H), 7.29~7.31 (m, 3H), 7.11~7.16 (m, 5H), 6.80~6.96 (m, 3H), 5.87 (br, 1H), 4.28~4.60 (m, 2H), 2.92 (d, J = 5.2 Hz, 3H), 2.78 (m, 3H). | 547 |
| 54 | 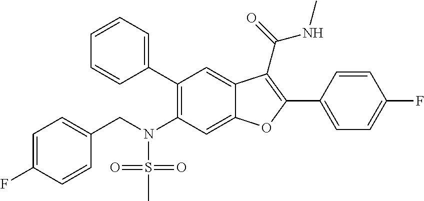 | 6-[(4- fluorobenzyl)(methyl- sulfonyl)amino]-2-(4- fluorophenyl)-N- methyl-5-phenyl-1- benzofuran-3- carboxamide | 7.88~7.91 (m, 2H), 7.69 (s, 1H), 7.39~7.40 (m, 3H), 7.35~7.36 (m, 2H), 7.33~7.34 (m, 1H), 7.16~7.20 (m, 2H), 6.97~7.01 (m, 2H), 6.86~6.90 (m, 2H), 6.02 (br s, 1H), 4.20~4.60 (br ABq, 2H), 2.96 (d, J = 4.8 Hz, 3H), 2.74 (s, 3H). | 547 |
| 55 | 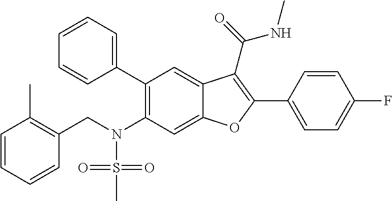 | 2-(4-fluorophenyl)- N-methyl-6-[(2- methylbenzyl) (methylsulfonyl) amino]-5-phenyl-1- benzofuran-3- carboxamide | 7.91~7.90 (m, 2H), 7.52~7.23 (m, 2H), 7.21~7.18 (m, 3H), 7.14~7.03 (m, 5H), 6.93~6.91 (m, 2H), 6.88~6.75 (m, 1H), 5.85 (s, 1H), 4.43 (m, 2H), 2.89 (s, 3H), 2.81 (s, 3H), 1.81 (s, 3H). | 543 |
| 56 | 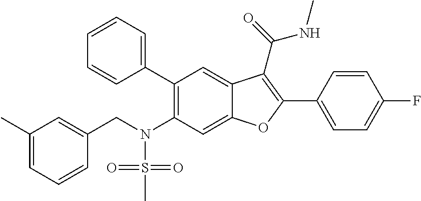 | 2-(4-fluorophenyl)- N-methyl-6-[(3- methylbenzyl) (methylsulfonyl) amino]-5-phenyl-1- benzofuran-3- carboxamide | 7.89~7.85 (m, 2H), 7.63 (s, 1H), 7.34~7.27 (m, 6H), 7.14~7.10 (m, 2H), 7.05~7.00 (m, 2H), 6.98~6.74 (m, 2H), 5.82 (s, 1H), 4.43~4.18 (m, 2H), 2.91 (s, 3H), 2.63 (s, 3H), 2.18 (s, 3H). | 543 |
| 57 | 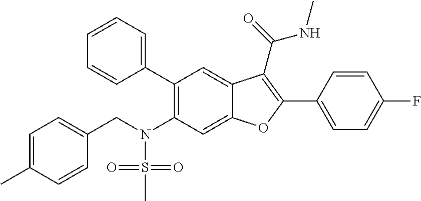 | 2-(4-fluorophenyl)- N-methyl-6-[(4- methylbenzyl) (methylsulfonyl) amino]-5-phenyl-1- benzofuran-3- carboxamide | 7.89~7.85 (m, 2H), 7.63 (s, 1H), 7.34~7.27 (m, 6H), 7.14~7.10 (m, 2H), 7.05~7.00 (m, 2H), 6.98~6.74 (m, 2H), 5.79 (s, 1H), 4.46~4.13 (m, 2H), 2.91 (s, 3H), 2.63 (s, 3H), 2.23 (s, 3H). | 543 |
| 58 | 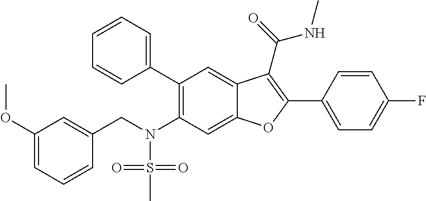 | 2-(4-fluorophenyl)-6- [(3-methoxybenzyl) (methylsulfonyl) amino)-N-methyl-5- phenyl-1-benzofuran- 3-carboxamide | 7.91~7.89 (m, 2H), 7.69 (s, 1H), 7.42~7.33 (m, 6H), 7.19~7.10 (m, 3H), 6.79~6.77 (m, 1H), 6.61~6.59 (m, 2H), 5.91 (s, 1H), 4.52~4.21 (m, 2H), 3.69 (s, 3H), 2.96 (s, 3H), 2.72 (s, 3H). | 559 |
| 59 | 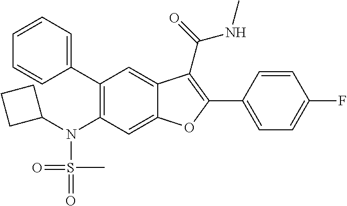 | 6-[cyclobutyl (methylsulfonyl) amino]-2-(4- fluorophenyl)-N- methyl-5-phenyl-1- benzofuran-3- carboxamide | 7.92~7.96 (m, 2H), 7.75 (d, J = 10.8 Hz, 1H), 7.56 (d, J = 9.6 Hz, 1H), 7.37~7.49 (m, 5H), 7.19 (t, J = 8.4 Hz, 2H), 5.95 (d, J = 3.6 Hz, 1H), 3.37~3.49 (m, 2H), 2.98 (d, J = 4.8 Hz, 3H), 2.78 (d, J = 12.8 Hz, 3H), 1.53~2.11 (m, 1H), 0.86~0.92 (m, 1H), 0.43 (s, 2H), 0.08 (d, J = 9.6 Hz, 1H). | 493 |
| 60 | 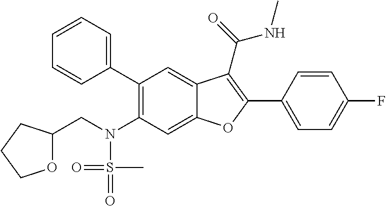 | 2-(4-fluorophenyl)- N-methyl-6- [(methylsulfonyl) (tetrahydrofuran-2- ylmethyl)amino]-5- phenyl-1-benzofuran- 3-carboxamide | 7.94~7.98 (m, 2H), 7.75 (s, 1H), 7.60 (d, J = 6.8 Hz, 1H), 7.37~7.45 (m, 5H), 7.20 (t, J = 8.8 Hz, 2H), 5.84 (d, J = 2.8 Hz, 1H), 3.80~3.86 (m, 1H), 3.70 (d, J = 6.8 Hz, 2H), 3.47~3.54 (m, 1H), 3.27 (s, 3H), 2.96 (d, J = 5.2 Hz, 3H), 2.69~2.77 (m, 1H), 1.77~1.82 (m, 2H), 1.62~1.67 (m, 1H), 1.19~1.26(m, 1H). | 523 |
| 61 | 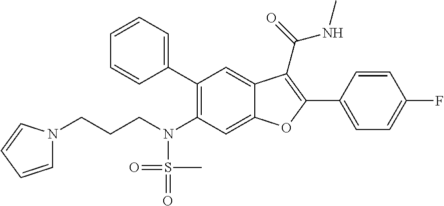 | 2-(4-fluorophenyl)- N-methyl-6- {(methylsulfonyl)[3- (1H-pyrrol-1- yl)propyl]amino}-5- phenyl-1-benzofuran- 3-carboxamide | 7.94~7.97 (m, 2H), 7.77 (s, 1H), 7.56 (s, 1H), 7.39~7.47 (m, 5H), 7.18~7.22 (m, 2H), 6.46 (s, 2H), 6.08 (s, 2H), 5.85 (s, 1H), 3.56 (m, 2H), 3.21~3.22 (m, 2H), 2.98~2.99 (d, J = 4.0 Hz, 3H), 2.80 (s, 3H), 1.84 (m, 2H). | 546 |
| 62 | 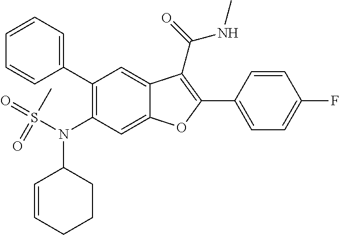 | 6-[cyclohex-2-en-1- yl(methylsulfonyl) amino]-2-(4- fluorophenyl)-N- methyl-5-phenyl-1- benzofuran-3- carboxamide | 7.88~7.91 (m, 2H), 7.66 (s, 1H), 7.55 (s, 1H), 7.47~7.47 (m, 2H), 7.31~7.37 (m, 3H), 7.10~7.14 (m, 2H), 5.81~5.82 (m, 2H), 4.28 (s, 1H), 2.98 (s, 1H), 2.90 (d, J = 4.8 Hz, 3H), 2.67 (s, 3H), 1.60~1.90 (m, 2H), 1.18~1.49 (m, 4H). | 519 |
| 63 | 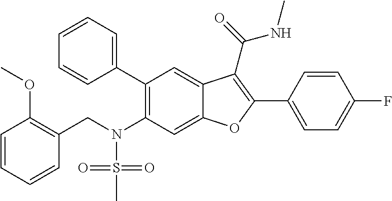 | 2-(4-fluorophenyl)-6- [(2-methoxybenzyl) (methylsulfonyl) amino]-N-methyl-5- phenyl-1-benzofuran- 3-carboxamide | 7.91~7.89 (m, 2H), 7.52 (s, 1H), 7.43 (s, 1H), 7.25~7.24 (m, 3H), 7.16~7.12 (m, 3H), 7.10~7.04 (m, 2H), 6.68~6.60 (m, 3H), 5.75 (s, 1H), 4.45~4.41 (m, 2H), 3.52 (s, 3H), 2.90 (s, 3H), 2.77 (s, 3H). | 559 |
| 64 | 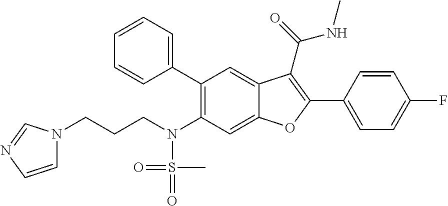 | 2-(4-fluorophenyl)-6- {[3-(1H-imidazol-1- yl)propyl] (methylsulfonyl) amino}-N-methyl-5- phenyl-1-benzofuran- 3-carboxamide | 8.62 (s, 1H), 7.96~7.94 (m, 2H), 7.85 (s, 1H), 7.65~7.60 (m, 3H), 7.43 (s, 1H), 7.42~7.40 (m, 4H), 7.27~7.24 (m, 2H), 3.63~3.62 (m, 2H), 3.43~3.42 (m, 2H), 3.29~3.27 (m, 2H), 3.11 (s, 3H), 2.91 (s, 3H). | 547 |
| 65 | 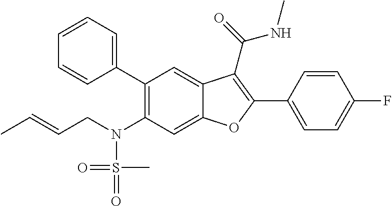 | 6-[(2E)-but-2-en-1- yl(methylsulfonyl) amino]-2-(4- fluorophenyl)-N- methyl-5-phenyl-1- benzofuran-3- carboxamide | Not available | 493 |
| 66 | 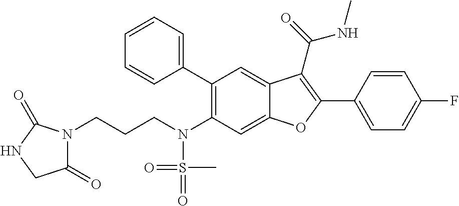 | 6-{[3-(2,5- dioxoimidazolidin-1- yl)propyl] (methylsulfonyl) amino}-2-(4- fluorophenyl)-N- methyl-5-phenyl-1- benzofuran-3- carboxamide | Not available | 579 |
| 67 | 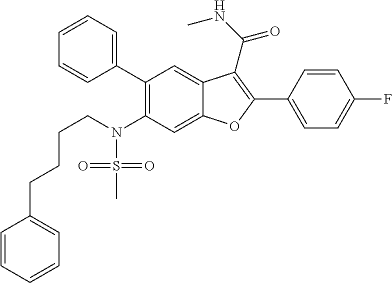 | 2-(4-fluorophenyl)- N-methyl-6- [(methylsulfonyl)(4- phenylbutyl)amino]- 5-phenyl-1- benzofuran-3- carboxamide | 7.89~7.92 (m, 2H), 7.68 (s, 1H), 7.48 (s, 1H), 7.31~7.40 (m, 5H), 7.06~7.16 (m, 5H), 6.99 (d, J = 6.4 Hz, 2H), 5.77 (s, 1H), 3.04~3.20 (m, 2H), 2.92 (d, J = 4.8 Hz, 3H), 2.70 (s, 3H), 2.36~2.45 (m, 2H), 1.33~1.42 (m, 4H). | 571 |
| 68 | 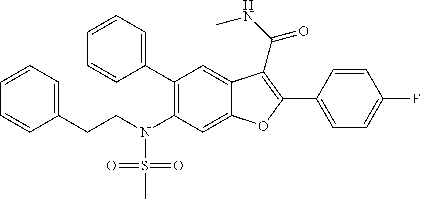 | 2-(4-fluorophenyl)- N-methyl-6- [(methylsulfonyl)(2- phenylethyl)amino]- 5-phenyl-1- benzofuran-3- carboxamide | 7.90~7.86 (m, 2H), 7.70 (s, 1H), 7.44~7.43 (m, 3H), 7.37~7.30 (m, 3H), 7.18~7.09 (m, 5H), 6.98~6.96 (m, 2H), 5.99 (s, 1H), 3.60~3.18 (m, 2H), 2.77 (s, 3H), 2.64~2.60 (m, 2H), 2.32 (s, 3H). | 543 |

### Example 69

### 2-(4-fluorophenyl)-N-methyl-5-phenyl-6-[(1-phenylethyl)amino]-1-benzofuran-3-carboxamide

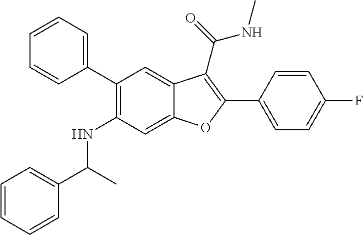

### Steps 1-3

Steps 1-3 were performed in accordance with Example 1, Steps 1-3.

### Step 4: ethyl 2-(4-fluorophenyl)-5-phenyl-6-[(1-phenylethyl)amino]-1-benzofuran-3-carboxylate

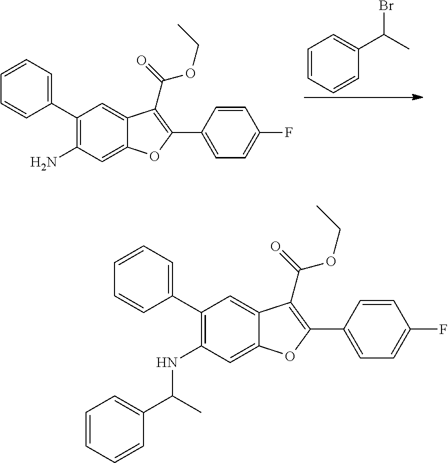

A mixture of the product of Step 3 (400 mg, 1.06 mmol), (1-bromoethyl)benzene (197 mg, 1.06 mmol) and Cs₂CO₃ (7.8 g, 24 mmol) in dry DMF (100 mL) was stirred at 140° C. for 4 hours. After the mixture was concentrated, the residue was diluted with DCM, washed with water, dried over Na₂SO₄ and concentrated. The residue was purified by prep-TLC to give the product (200 mg, yield: 39%).

¹H-NMR (400 MHz, CDCl₃) δ 7.90˜7.88 (m, 2H), 7.62 (s, 1H), 7.47˜7.45 (m, 2H), 7.45˜7.44 (m, 1H), 7.26˜7.25 (m, 5H), 7.18˜7.17 (m, 2H), 7.04˜7.03 (m, 2H), 6.45 (s, 1H), 4.42˜4.41 (m, 1H), 4.28˜4.26 (q, J=8.0 Hz, 2H), 1.36˜134 (d, J=8.0 Hz, 3H), 1.26˜1.24 (t, J=8.0 Hz, 3H). MS (M+H)⁺: 480.

### Step 5: 2-(4-fluorophenyl)-5-phenyl-6-[(1-phenylethyl)amino]-1-benzofuran-3-carboxylic acid

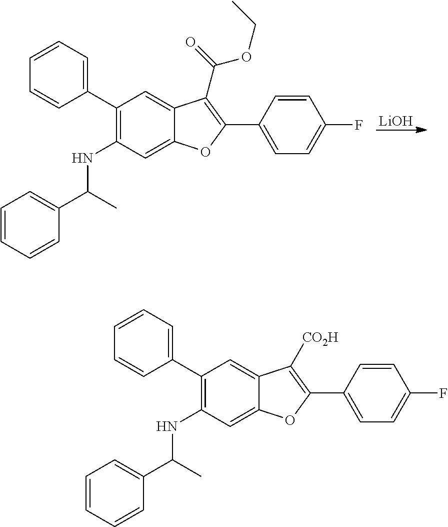

The product (110 mg, yield: 58.4%) was prepared in an analogous manner to Example 13 using the general procedure in Example 13, Step 4. The crude product was used in the next step without further purification.

¹H-NMR (400 MHz, CDCl₃) δ 7.93˜7.89 (m, 2H), 7.70 (s, 1H), 7.46˜7.45 (m, 4H), 7.42˜7.40 (m, 1H), 7.38˜7.35 (m, 4H), 7.07˜7.03 (m, 3H), 6.50 (s, 1H), 4.44˜4.39 (m, 1H), 1.37˜1.36 (d, J=4.0 Hz, 3H). MS (M+H)⁺: 452.

### Step 6: 2-(4-fluorophenyl)-N-methyl-5-phenyl-6-[(1-phenylethyl)amino]-1-benzofuran-3-carboxamide

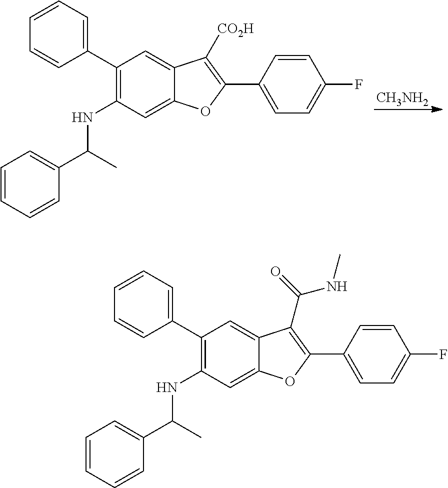

Example 69 (20 mg, yield: 48.6%) was prepared according to the general procedure in Example 1, Step 6.

¹H-NMR (400 MHz, CDCl₃) δ 7.82˜7.78 (m, 2H), 7.45˜7.44 (m, 4H), 7.36˜7.35 (m, 2H), 7.27˜7.25 (m, 4H), 7.18˜7.16 (m, 2H), 7.05˜7.01 (m, 2H), 6.48 (s, 1H), 5.72 (s, 1H), 4.44˜4.39 (m, 1H), 2.89˜2.87 (s, 3H), 1.37˜1.35 (d, J=8.0 Hz, 3H). MS (M+H)⁺: 465.

### Example 70

### 2-(4-fluorophenyl)-N-methyl-6-({2-[methyl(phenyl)amino]ethyl}amino)-5-phenyl-1-benzofuran-3-carboxamide

Example 70 was prepared according to the general procedures of Example 69.

| Ex- | | | | MS |
|---|---|---|---|---|
| ample | Structure | Name | ¹H-NMR (400 MHz, CDCl₃) δ | (M + H)⁺ |
| 70 | 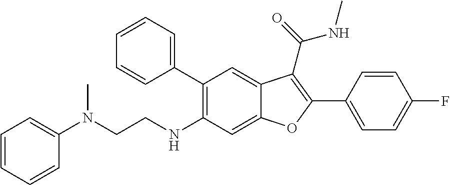 | 2-(4-fluorophenyl)-N- methyl-6-({2- [methyl(phenyl)amino] ethyl}amino)-5-phenyl- 1-benzofuran-3- carboxamide | 7.91~8.08 (m, 2H), 7.65 (s, 1H), 7.25~7.35 (m, 5H), 7.10~7.15 (m, 4H), 6.71 (s, 1H) 6.60~6.70 (m, 3H), 4.25~4.31 (m, 2H), 3.41~3.50 (m, 2H), 2.75 (s, 3H), 1.30~1.33 (t, J = 12.0 Hz, 3H). | 494 |

### Example 71

### 2-(4-fluorophenyl)-N-methyl-6-[methyl(1-phenylethyl)amino]-5-phenyl-1-benzofuran-3-carboxamide

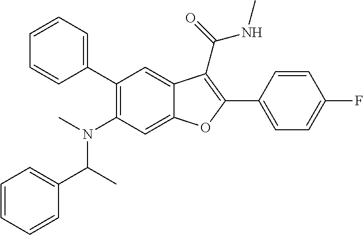

### Steps 1-4

Steps 1-4 were performed in accordance with Example 69, Steps 1-4.

### Step 5: ethyl 2-(4-fluorophenyl)-6-[methyl(1-phenylethyl)amino]-5-phenyl-1-benzofuran-3-carboxylate

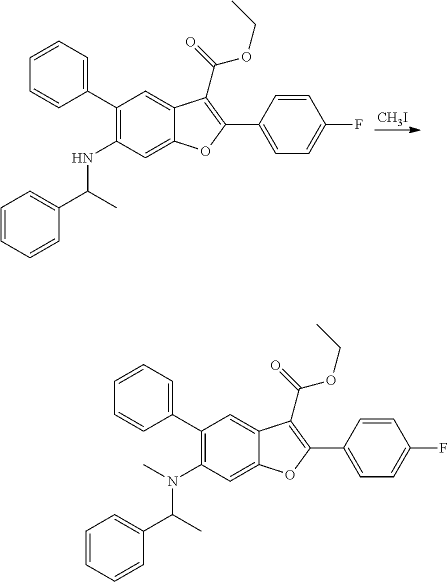

The product of Step 4 (62 mg, 0.13 mmol), CH₃I (29 mg, 0.20 mmol), K₂CO₃ (37 mg, 0.27 mmol) in DMF (2 mL) was stirred at 90° C. for 16 hours. The mixture was quenched with water, diluted with DCM, dried over Na₂SO₄, filtered, and the solvent was evaporated. The residue was purified by prep-TLC to give pure compound product (49 mg, yield: 77.7%) as a yellow solid.

¹H-NMR (400 MHz, CDCl₃) δ 7.90˜7.88 (m, 2H), 7.62 (s, 1H), 7.47˜7.45 (m, 2H), 7.45˜7.44 (m, 1H), 7.26˜7.25 (m, 5H), 7.18˜7.17 (m, 2H), 7.04˜7.03 (m, 2H), 6.45 (s, 1H), 4.33˜4.28 (q, J=2.0 Hz, 2H), 4.17˜4.12 (m, 1H), 2.50 (s, 3H), 1.32˜1.27 (t, J=2.0 Hz, 3H), 1.32˜1.34 (d, J=0.8 Hz, 3H). MS (M+H)⁺: 494.

### Step 6: 2-(4-fluorophenyl)-6-[methyl(1-phenylethyl)amino]-5-phenyl-1-benzofuran-3-carboxylic acid

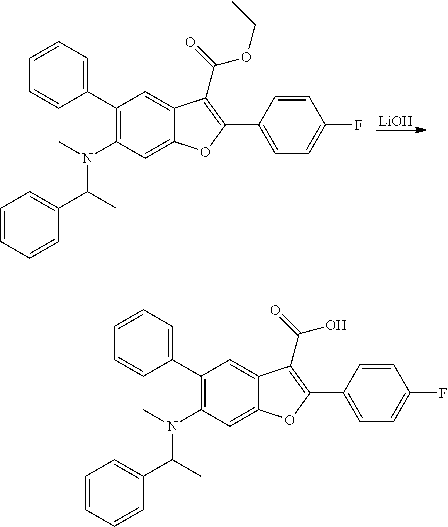

The carboxylic acid (75 mg, yield: 90%) was prepared in an analogous manner to Example 13 using the general procedure in Example 13, Step 4. The carboxylic acid was used in the next step without further purification.

¹H-NMR (400 MHz, CDCl₃) δ 7.90˜7.88 (m, 2H), 7.62 (s, 1H), 7.47˜7.45 (m, 2H), 7.45˜7.44 (m, 1H), 7.26˜7.25 (m, 5H), 7.18˜7.17 (m, 2H), 7.04˜7.03 (m, 2H), 6.45 (s, 1H), 4.17˜4.12 (m, 1H), 2.50 (s, 3H), 1.32˜1.34 (d, J=0.8 Hz, 3H). MS (M+H)⁺: 466.

### Step 7: 2-(4-fluorophenyl)-N-methyl-6-[methyl(1-phenylethyl)amino]-5-phenyl-1-benzofuran-3-carboxamide

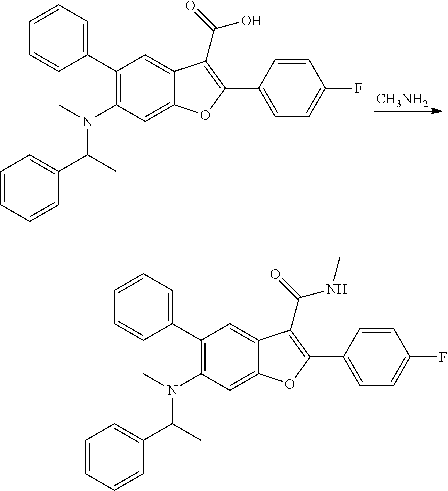

The product (30 mg, yield: 38.9%) was prepared according to the general procedure in Example 1, Step 6.

¹H-NMR (400 MHz, CDCl₃) δ 7.86˜7.83 (m, 2H), 7.66 (s, 1H), 7.50˜7.45 (m, 4H), 7.34˜7.33 (m, 2H), 7.25˜7.20 (m, 2H), 7.17˜7.13 (m, 2H), 6.95˜6.93 (m, 2H), 6.96 (s, 1H), 4.55 (m, 1H), 2.93 (s, 3H), 2.85 (s, 3H), 1.35 (s, 3H). MS (M+H)⁺: 479.

### Example 72

### ethyl[2-(4-fluorophenyl)-3-(methylcarbamoyl)-5-phenyl-1-benzofuran-6-yl]{2-[methyl(phenyl)amino]ethyl}carbamate

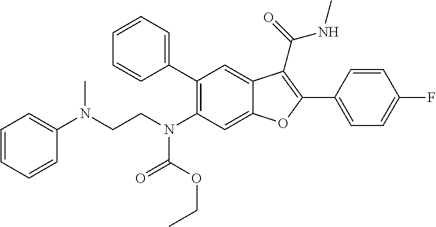

### Steps 1-3

Steps 1-3 were performed in accordance with Example 1, Steps 1-3.

### Step 4: ethyl 6-[(ethoxycarbonyl)amino]-2-(4-fluorophenyl)-5-phenyl-1-benzofuran-3-carboxylate

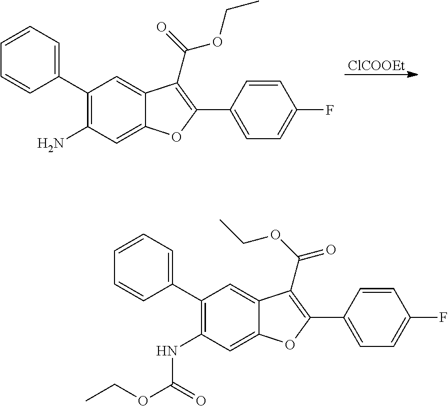

A mixture of the product of Step 3 (64 mg, 0.17 mmol), EtOOCCl (22 mg, 0.21 mmol), Py (23 mg, 0.31 mmol) in DCM (3 mL) was stirred at RT for 2 hours. The mixture was quenched with H₂O, diluted with DCM, dried over Na₂SO₄, filtered, and the solvent was evaporated. The residue was purified by prep-TLC to give pure carbamate (63 mg, yield: 83.3%) as a white solid.

¹H-NMR (400 MHz, CDCl₃) δ 8.02˜8.00 (m, 2H), 7.78 (s, 1H), 7.49˜7.47 (m, 2H), 7.45˜7.34 (m, 3H), 7.14˜7.09 (m, 2H), 6.67 (m, 1H), 4.34˜4.30 (q, J=1.6 Hz, 2H), 4.16˜4.11 (q, J=2.0 Hz, 2H), 2.18˜2.14 (t, J=1.6 Hz, 3H), 2.13˜1.98 (t, J=2.0 Hz, 3H). MS (M+H)⁺: 448.

### Step 5: ethyl 6-[(ethoxycarbonyl){2-[methyl(phenyl)amino]ethyl}amino]-2-(4-fluorophenyl)-5-phenyl-1-benzofuran-3-carboxylate

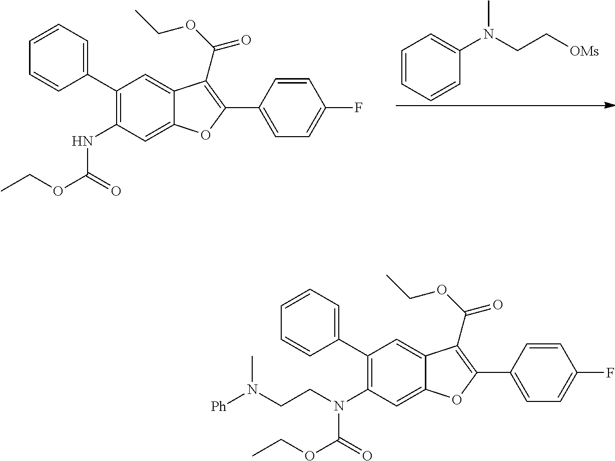

The product of Step 4 (474 mg, 1.06 mmol), 2-(methyl(phenyl)amino)ethyl methanesulfonate (243 mg, 1.06 mmol) and Cs₂CO₃ (7.8 g, 24 mmol) in dry DMF (100 mL) was stirred at 140° C. for 4 hours. After the mixture was concentrated, the residue was diluted with DCM, washed with water, dried over Na₂SO₄ and concentrated. The residue was purified by prep-TLC to give the desired amino carbamate (335 mg, yield: 54.6%). MS (M+H)⁺: 581.

### Step 6: 6-[(ethoxycarbonyl){2-[methyl(phenyl)amino]ethyl}amino]-2-(4-fluorophenyl)-5-phenyl-1-benzofuran-3-carboxylic acid

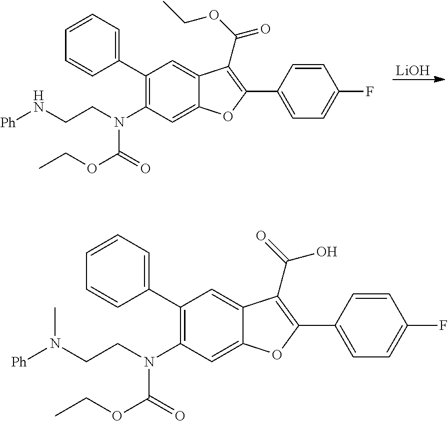

The product of Step 5 (25 mg, yield: 90%) was prepared in an analogous manner to Example 13 using the general procedure in Example 13, Step 4. The carboxylic acid was used directly in the next step without further purification.

### Step 7: ethyl[2-(4-fluorophenyl)-3-(methylcarbamoyl)-5-phenyl-1-benzofuran-6-yl]{2-[methyl(phenyl)amino]ethyl}carbamate

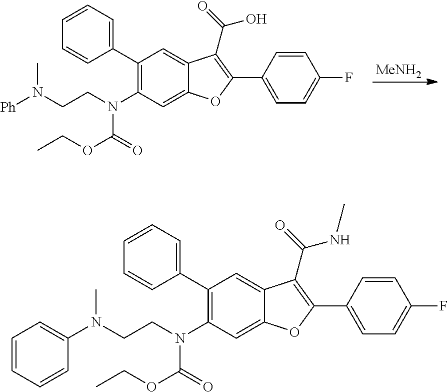

Example 72 (15 mg, yield: 48.7%) was prepared according to the general procedure in Example 1, Step 6.

¹H-NMR (400 MHz, CDCl₃) δ 7.89˜7.87 (m, 2H), 7.65 (s, 1H), 7.40˜7.36 (m, 2H), 7.32˜7.20 (m, 6H), 7.19˜7.18 (m, 3H), 7.15˜7.10 (m, 2H), 6.09 (m, 1H), 4.09˜4.04 (m, 2H), 3.35˜3.36 (m, 2H), 3.19˜3.07 (m, 2H), 2.97˜2.89 (m, 6H), 1.21˜1.10 (m, 3H). MS (M+H)⁺: 566.

### Example 73

### 2-(4-fluorophenyl)-N-methyl-6-(N-methyl-N-phenylglycyl)amino]-5-phenyl-1-benzofuran-3-carboxamide

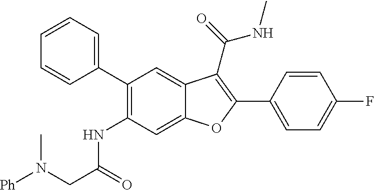

### Steps 1-2

Steps 1-2 were performed in accordance with Example 1, Steps 1-2.

### Step 3: ethyl 2-(4-fluorophenyl)-6-(N-methyl-N-phenylglycyl)amino]-5-phenyl-1-benzofuran-3-carboxylate

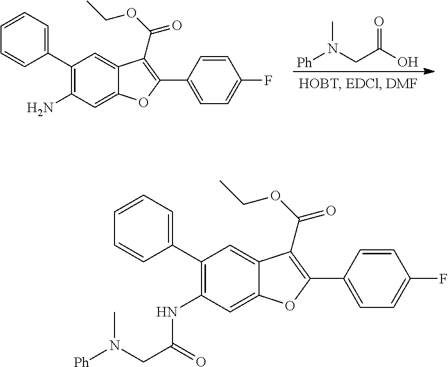

The amide (75 mg, yield: 50%) was prepared from the product of Step 2 according to the general procedure in Example 1, Step 6.

¹H-NMR (400 MHz, CDCl₃) δ 8.41˜8.48 (m, 2H), 8.01˜8.09 (m, 2H), 7.78 (s, 1H), 7.016˜7.15 (m, 8H), 6.71˜6.75 (m, 1H), 6.50 (t, J=12.0 Hz, 2H), 4.31˜4.35 (m, 2H), 3.24 (s, 3H), 2.61 (m, 2H), 1.30˜1.33 (t, J=12.0 Hz, 3H). MS (M+H)⁺: 523.

### Step 4: 2-(4-fluorophenyl)-6-(N-methyl-N-phenylglycyl)amino]-5-phenyl-1-benzofuran-3-carboxylic acid

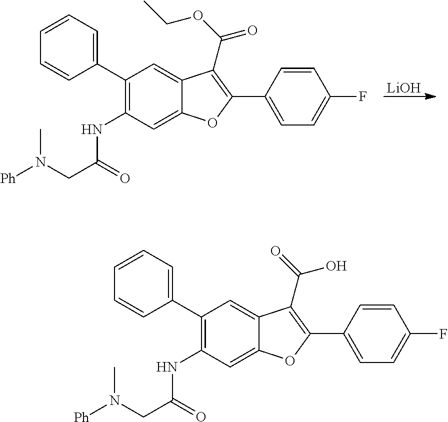

The carboxylic acid (50 mg, yield: 75%) was prepared in an analogous manner to Example 13 using the general procedure in Example 13, Step 4. The carboxylic acid was used in the next step without further purification.

### Step 5: 2-(4-fluorophenyl)-N-methyl-6-(N-methyl-N-phenylglycyl)amino]-5-phenyl-1-benzofuran-3-carboxamide

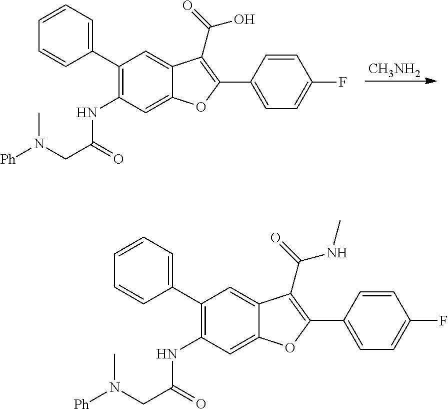

The amide (35 mg, yield: 78%) was prepared according to the general procedure in Example 1, Step 6.

¹H-NMR (400 MHz, CDCl₃) δ 8.85 (s, 3H), 8.71 (s, 3H), 7.81˜7.89 (m, 2H), 7.55 (s, 1H), 7.23˜7.25 (m, 5H), 7.01˜7.12 (m, 2H), 6.71˜6.75 (m, 1H), 6.50 (d, J=12.0 Hz, 2H), 5.71˜5.75 (m, 2H), 3.78 (s, 3H), 2.58 (s, 3H). MS (M+H)⁺: 508.

### Example 74

### 2-(4-fluorophenyl)-N-methyl-6-[methyl(N-methyl-N-phenylglycyl)amino]-5-phenyl-1-benzofuran-3-carboxamide

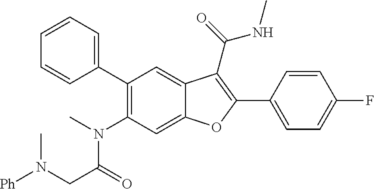

### Steps 1-3

Steps 1-3 were performed in accordance with Example 73, Steps 1-3.

### Step 4: ethyl 2-(4-flourophenyl)-6-[methyl(N-methyl-N-phenylglycyl)amino]-5-phenyl-1-benzofuran-3-carboxylate

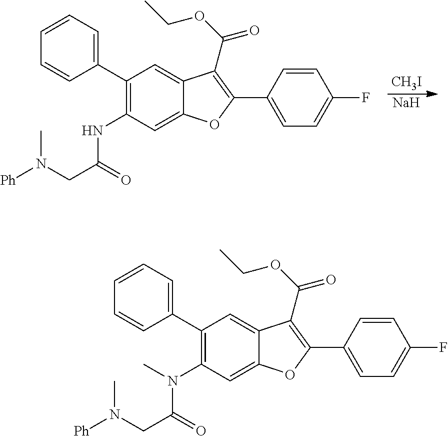

The alkylated amide (90 mg, yield: 90%) was prepared in an analogous manner to the compound prepared in Example 1, Step 4.

¹H-NMR (400 MHz, CDCl₃) δ 8.09 (s, 1H), 8.01˜8.05 (m, 2H), 7.36˜7.45 (m, 6H), 7.13˜7.18 (m, 2H), 6.96˜7.02 (m, 2H), 6.53˜6.61 (m, 1H), 6.53˜6.61 (t, J=4.0 Hz, 2H), 4.31˜4.39 (m, 2H), 3.58˜3.66 (m, 2H), 3.24 (s, 3H), 2.70 (s, 3H), 1.30˜1.33 (t, J=12.0 Hz, 3H). MS (M+H)⁺: 537.

### Step 5: 2-(4-fluorophenyl)-6-[methyl(N-methyl-N-phenylglycyl)amino]-5-phenyl-1-benzofuran-3-carboxylic acid

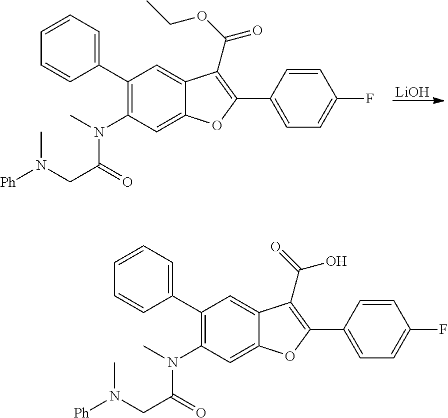

The carboxylic acid (85 mg, yield: 95%) was prepared in an analogous manner to Example 13 using the general procedure in Example 13, Step 4. The carboxylic acid was used in the next step without further purification.

### Step 6: 2-(4-fluorophenyl)-N-methyl-6-[methyl(N-methyl-N-phenylglycyl)amino]-5-phenyl-1-benzofuran-3-carboxamide

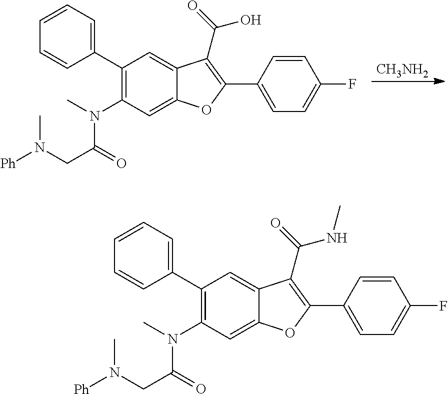

The amide was prepared in an analogous manner to Example 1, Step 6 (25 mg, yield: 68%).

¹H-NMR (400 MHz, CDCl₃) δ 7.89˜7.91 (m, 2H), 7.86 (s, 1H), 7.39˜7.42 (m, 4H), 7.34˜7.38 (m, 2H), 7.13˜7.18 (m, 2H), 7.00˜7.09 (m, 2H), 6.55˜6.57 (m, 1H), 6.16 (d, J=4.0 Hz, 2H), 5.71˜5.73 (m, 1H), 3.48˜3.56 (m, 2H), 3.24 (s, 3H), 2.94 (d, J=8.0 Hz, 3H), 2.69 (s, 3H). MS (M+H)⁺: 522.

### Examples 75-76

Examples 75 and 76 were prepared according to the general procedures of Example 74.

| | | | | MS |
|---|---|---|---|---|
| Example | Structure | Name | ¹H-NMR (400 MHz, CDCl₃) δ | (M + H)⁺ |
| 75 | 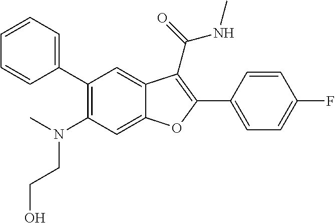 | 2-(4-fluorophenyl)-6- [(2- hydroxyethyl)(methyl) amino]-N-methyl-5- phenyl-1-benzofuran-3- carboxamide | 7.84~7.88 (m, 2H), 7.70 (s, 1H), 7.62 (s, 1H), 7.33~7.41 (m, 5H), 7.13~7.19 (m, 2H), 5.93 (br, 1H), 3.60 (s, 2H), 3.38 (s, 2H), 2.97 (s, 3H), 2.91 (d, J = 4.8 Hz, 3H). | 419 |
| 76 | 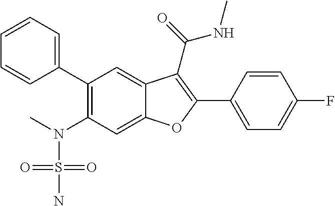 | 2-(4-fluorophenyl)-N- methyl-6- [methyl(sulfamoyl) amino]-5-phenyl-1- benzofuran-3- carboxamide | 7.84~7.88 (m, 2H), 7.32~7.43 (m, 6H), 7.07~7.19 (m, 2H), 6.85 (s, 1H), 5.89 (br, 1H), 2.92 (d, J = 4.8 Hz, 3H), 2.79 (s, 3H). | 454 |

### Example 77

### 2-(4-fluorophenyl)-N-methyl-6-[(4S,5R)-4-methyl-2-oxo-5-phenyl-1,3-oxazolidin-3-yl]-5-phenyl-1-benzofuran-3-carboxamide

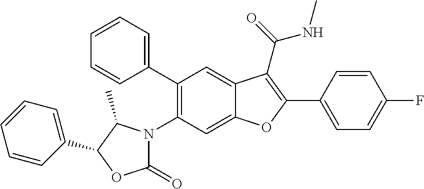

### Steps 1-3

Steps 1-3 were performed in accordance with Example 1, Steps 1-3.

### Step 4: ethyl 2-(4-fluorophenyl)-6-iodo-5-phenyl-1-benzofuran-3-carboxylate

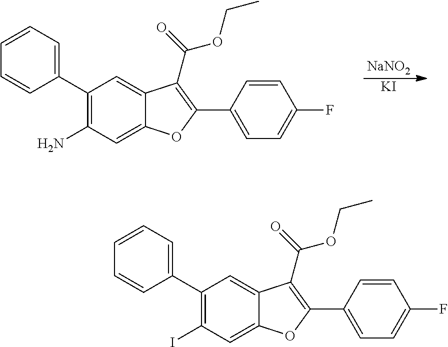

A solution of the product of Step 3 (100 mg, 0.27 mmol) in 30% H₂SO₄ aqueous solution was cooled at 0° C. Then the solution of NaNO₂ in 1 mL H₂O was added dropwise to amine solution over a period of 1 minute with keeping the temperature at 0° C. The resulting mixture was stirred for an additional 30 minutes at 0° C. An aqueous solution of KI was added dropwise over 5 minutes. The reaction mixture was stirred for 3 hours at RT, giving a dark brown solution. The solution was extracted with EtOAc. The organic layer was washed with Na₂SO₃ solution and concentrated to give the crude iodide (40 mg, yield: 31%).

¹H-NMR (400 MHz, CDCl₃) δ 8.12 (s, 1H), 8.06˜8.10 (m, 2H), 7.99 (s, 1H), 7.38˜7.48 (m, 5H), 7.17˜7.22 (m, 2H), 4.39 (q, J=7.2 Hz, 2H), 1.35 (t, J=7.2 Hz, 3H). MS (M+H)⁺: 487.

### Step 5: ethyl 2-(4-fluorophenyl)-6-[(4S,5R)-4-methyl-2-oxo-5-phenyl-1,3-oxazolidin-3-yl]-5-phenyl-1-benzofuran-3-carboxylate

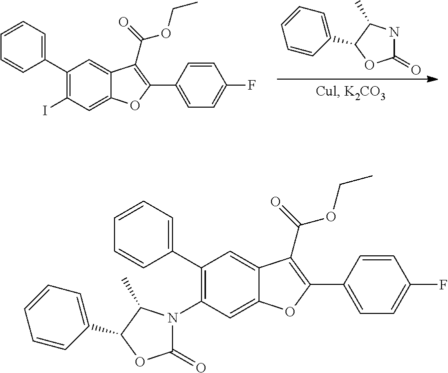

The iodide (30 mg, 0.06 mmol), (4S,5R)-4-methyl-5-phenyloxazolidin-2-one (17 mg, 0.9 mmol), CuI (15 mg, 0.08 mmol) and K₂CO₃ (20 mg, 0.14 mmol) in dry nitrobenzene (1 mL) was heated to 180° C. for 6 hours. When TLC showed the reaction was completed, H₂O was added to the mixture and the aqueous phase was extracted by EtOAc. The combined organic phase was washed with brine, dried over Na₂SO₄ and concentrated under reduced pressure. The residue was purified by prep-TLC to give the N-aryl oxizolidinone (10 mg, yield: 30%).

¹H-NMR (400 MHz, CDCl₃) δ 8.00˜8.02 (m, 3H), 7.99 (s, 1H), 7.39˜7.55 (m, 5H), 7.07˜7.27 (m, 7H), 5.26 (d, J=8.0 Hz, 1H), 4.33 (q, J=7.2 Hz, 2H), 3.61 (br s, 1H), 1.31 (t, J=7.2 Hz, 3H), 0.45 (d, J=6.8 Hz, 3H). MS (M+H)⁺: 536.

### Step 6: 2-(4-fluorophenyl)-6-[(4S,5R)-4-methyl-2-oxo-5-phenyl-1,3-oxazolidin-3-yl]-5-phenyl-1-benzofuran-3-carboxylic acid

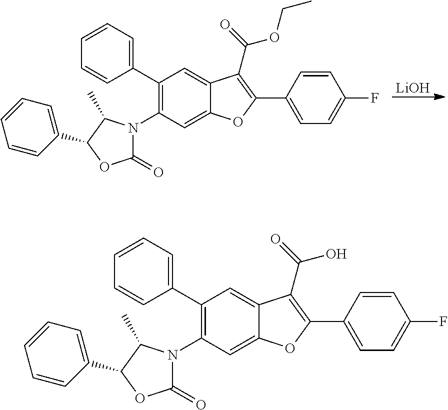

To a stirred solution of ester (40 mg, 0.07 mmol) in dioxane/H₂O (1:1, 2 mL) was added LiOH (20 mg, 0.48 mmol), and the mixture was stirred at 100° C. for 3 hours. The mixture was concentrated in vacuo. The residue was dissolved in H₂O, 1N HCl was added until pH to 3, and the mixture was extracted with EtOAc. The organic solvent was washed with brine, dried over Na₂SO₄ and filtered, and the solvent was evaporated. The solvent was removed by distillation to provide the crude carboxylic acid (35 mg, yield: 92%). It was used for the next step without further purification.

### Step 7: 2-(4-fluorophenyl)-N-methyl-6-[(4S,5R)-4-methyl-2-oxo-5-phenyl-1,3-oxazolidin-3-yl]-5-phenyl-1-benzofuran-3-carboxamide

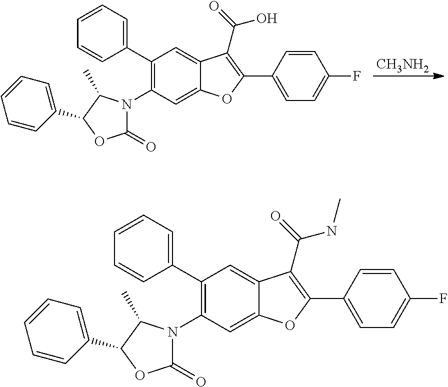

A solution of carboxylic acid (35 mg, 0.07 mmol), HOBT (40 mg, 0.30 mmol) and EDCI (50 mg, 0.32 mmol) in dry DMF (2 mL) was stirred at RT. After 30 minutes, Et₃N (0.2 mL) and CH₃NH₂ (HCl salt, 40 mg, 0.59 mmol) was added to the mixture, and the mixture was stirred overnight. After the solvent was removed, H₂O was added, and the mixture was extracted with EtOAc. The combined organic layer was washed with H₂O, brine and concentrated. The residue was purified by prep-TLC to give the product of Example 77 (20 mg., yield: 56%).

¹H-NMR (400 MHz, CDCl₃) δ 7.86˜7.89 (m, 2H), 7.74 (s, 1H), 7.52 (s, 1H), 7.40˜7.42 (m, 5H), 7.25˜7.26 (m, 3H), 7.06˜7.14 (m, 4H), 5.84 (br s, 1H), 5.25 (d, J=8.0 Hz, 1H), 3.62 (br s, 1H), 2.91 (d, J=4.8 Hz, 3H), 0.44 (d, J=6.8 Hz, 3H). MS (M+H)⁺: 521.

### Example 78

### 5-(2-fluorophenyl)-2-(4-fluorophenyl)-N-methyl-6-[methyl(methylsulfonyl)amino]-1-benzofuran-3-carboxamide

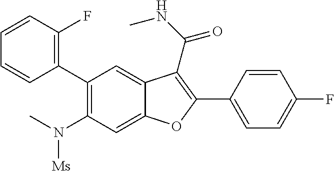

### Step 1: ethyl 5-(2-fluorophenyl)-2-(4-fluorophenyl)-6-nitro-1-benzofuran-3-carboxylate

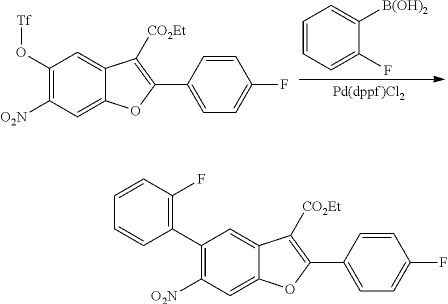

2-Fluorophenylboronic acid (obtained according to procedure in WO 2004/041201 A2; 283 mg, 2.10 mmol) and K₃PO₄.3H₂O (556 mg, 2.10 mmol) were added to a suspension of triflate (described in Example 1) (500 mg, 1.05 mmol) in dry DMF (2 mL) under N₂. Then Pd(dppf)Cl₂ (5 mg, 0.08 mmol) was added to the mixture under N₂. The reaction mixture was heated to 80° C. for 6 hours. The mixture was cooled, diluted with water and extracted with EtOAc. The combined organic phases were washed with brine, dried over Na₂SO₄, filtered and evaporated. The crude product was purified by column to give pure aryl fluoride (250 mg, yield: 55%).

¹H-NMR (400 MHz, CDCl₃) δ 8.02 (s, 1H), 8.00˜8.01 (m, 3H), 7.31˜7.35 (m, 2H), 7.20˜7.22 (m, 3H), 7.03˜7.05 (m, 1H), 4.30˜4.36 (dd, J=8.0 Hz, 2H), 1.27˜1.31 (m, 3H). MS (M+H)⁺: 424.

### Step 2: ethyl 6-amino-5-(2-fluorophenyl)-2-(4-fluorophenyl)-1-benzofuran-3-carboxylate

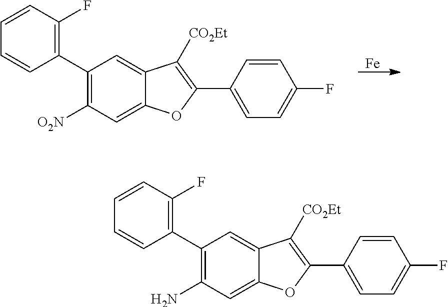

A mixture of nitro arene (250 mg, 0.59 mmol), Fe (264 mg, 4.70 mmol) and NH₄Cl (475 mg, 8.85 mmol) in H₂O/MeOH/THF (2 mL/2 mL/2 mL) was refluxed for 3 hours. Then, H₂O was added to quench the reaction, which was filtered and extracted with EtOAc, washed with brine and dried over Na₂SO₄. The solvent was removed by distillation. After purification by column, the desired aninline was obtained (180 mg, yield: 77%).

¹H-NMR (400 MHz, CDCl₃) δ 7.94˜7.97 (m, 2H), 7.74 (s, 1H), 7.32˜7.35 (m, 2H), 7.05˜7.20 (m, 4H), 6.67 (s, 1H), 4.26˜4.30 (dd, J=8.0 Hz, 2H), 1.18˜1.27 (m, 3H). MS (M+H)⁺: 394.

### Step 3: ethyl 5-(2-fluorophenyl)-2-(4-fluorophenyl)-6-[(methylsulfonyl)amino]-1-benzofuran-3-carboxylate

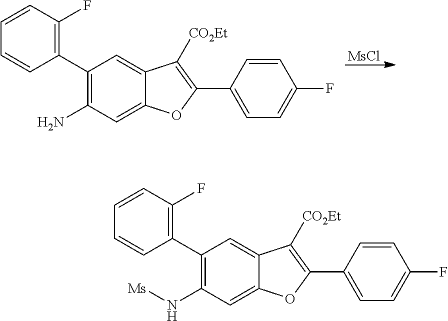

MsCl (65 mg, 0.60 mmol) was added to a solution of aniline (180 mg, 0.50 mmol) and pyridine (79 mg, 1.00 mmol) in dry DCM (2 mL). The reaction mixture was stirred overnight at RT. After diluted with H₂O and extracted with DCM, the mixture was washed with brine, dried over Na₂SO₄ and filtered, and the solvent was evaporated under reduced pressure. The crude product was purified by prep-TLC to give sulfonamide (150 mg, yield: 75%).

¹H-NMR (400 MHz, CDCl₃) δ 7.94˜7.97 (m, 2H), 7.74 (s, 1H), 7.71 (s, 1H), 7.32˜7.35 (m, 2H), 7.05˜7.20 (m, 4H), 4.26˜4.30 (dd, J=8.0 Hz, 2H), 2.95 (s, 3H), 1.18˜1.27 (m, 3H). MS (M+H)⁺: 472.

### Step 4: ethyl 5-(2-fluorophenyl)-2-(4-fluorophenyl)-6-[methyl(methylsulfonyl)amino]-1-benzofuran-3-carboxylate

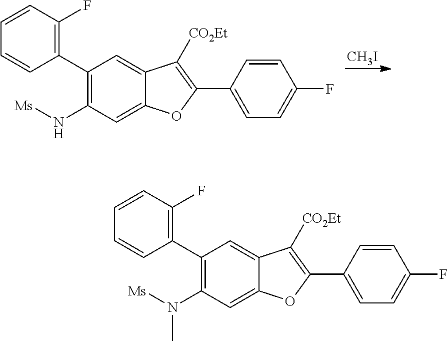

KI (4 mg, 0.02 mmol), K₂CO₃ (60 mg, 0.40 mmol), and CH₃I (113 mg, 0.80 mmol) were added to a solution of sulfonamide (100 mg, 0.20 mmol) in dry DMF (5 mL) under N₂. The mixture was heated to 80° C. overnight. The mixture was cooled, diluted with H₂O, and extracted with EtOAc; the organic solvent was washed with brine, dried over Na₂SO₄ and filtered; and the solvent was evaporated under reduced pressure. The crude was purified by prep-TLC and the desired alkyl sulfonamide was obtained (90 mg, yield: 87%).

¹H-NMR (400 MHz, CDCl₃) δ ppm 8.03˜8.05 (m, 2H), 8.01 (s, 1H), 7.63 (s, 1H), 7.37˜7.44 (m, 2H), 7.12˜7.27 (m, 4H), 4.34˜4.40 (dd, J=8.0 Hz, 2H), 3.23 (s, 3H), 2.48 (s, 3H), 1.34˜1.36 (m, 3H). MS (M+H)⁺: 486.

### Step 5: 5-(2-fluorophenyl)-2-(4-fluorophenyl)-6-[methyl(methylsulfonyl)amino]-1-benzofuran-3-carboxylic acid

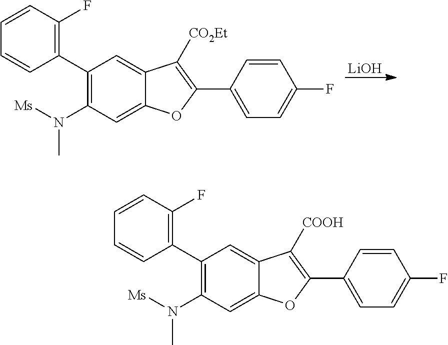

The ester (90 mg, 0.20 mmol) was dissolved in 1,4-dioxane (2 mL) and H₂O (2 mL). Then LiOH (84 mg, 2.00 mmol) was added to the solution, and the mixture was refluxed for 2 hours. After acidified with HCl (1 N) and extracted with EtOAc, the combined organic phases were washed with brine, dried over Na₂SO₄, filtered and evaporated to give the carboxylic acid (80 mg, yield: 90%). It was used for the next step without further purification.

### Step 6: 5-(2-fluorophenyl)-2-(4-fluorophenyl)-N-methyl-6-[methyl(methylsulfonyl)amino]-1-benzofuran-3-carboxamide

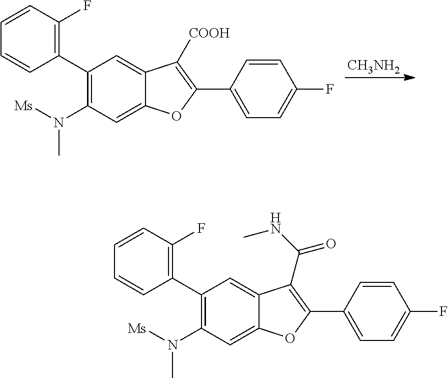

The carboxylic acid (75 mg, 0.16 mmol), HOBT (37 mg, 0.24 mmol) and EDCI (77 mg, 0.40 mmol) were dissolved in dry DMF (2 mL). The resulting solution was stirred for 30 minutes. Then, methanamine HCl salt (43 mg, 0.64 mmol) and Et₃N (73 mg, 0.72 mmol) was added to the mixture. After stirred overnight, the mixture was diluted with water and extracted with EtOAc. The combined organic phases were washed with brine, dried over Na₂SO₄, filtered and evaporated. The crude product was purified by prep-TLC to give pure Example 78 (35 mg, yield: 47%).

¹H-NMR (400 MHz, CDCl₃) δ 7.88˜7.92 (m, 2H), 7.74 (s, 1H), 7.60 (s, 1H), 7.34˜7.40 (m, 2H), 7.10˜7.24 (m, 4H), 5.92 (s, 1H), 3.20 (s, 3H), 2.94˜2.95 (d, J=4.0 Hz, 3H), 2.47 (s, 3H). MS (M+H)⁺: 471

### Examples 79-89

Examples 79-89 were prepared according to the general procedures of Example 78.

| | | | | MS |
|---|---|---|---|---|
| Example | Structure | Name | ¹H-NMR (400 MHz, CDCl₃) δ | (M + H)⁺ |
| 79 | 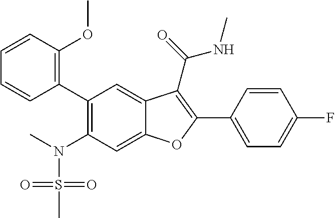 | 2-(4-fluorophenyl)-5- (2-methoxyphenyl)- N-methyl-6-[methyl (methylsulfonyl) amino]-1- benzofuran-3- carboxamide | 7.88~7.91 (m, 2H), 7.62 (s, 1H), 7.53 (s, 1H), 7.28~7.32 (m, 1H), 7.23 (d, J = 4.0 Hz, 1H), 7.09~7.13 (m, 2H), 6.97~6.99 (t, J = 8.0 Hz, 1H), 6.88~6.90 (t, J = 8.0 Hz, 1H), 5.79~5.80 (m, 1H), 3.69 (s, 3H), 3.11 (s, 3H), 2.89~2.91 (d, J = 8.0 Hz, 3H), 2.34 (s, 3H). | 483 |
| 80 | 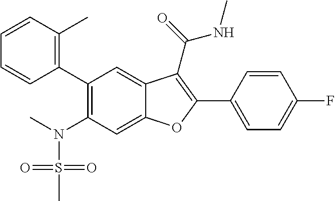 | 2-(4-fluorophenyl)- N-methyl-6-[methyl (methylsulfonyl) amino]-5-(2- methylphenyl)-1- benzofuran-3- carboxamide | 7.91 (t, J = 4.2 Hz, 2H), 7.63 (d, J = 3.6 Hz, 1H), 7.57 (d, J = 4.0 Hz, 1H), 7.13~7.27 (m, 6H), 5.92 (s, 1H), 3.11 (d, J = 2.8 Hz, 3H), 2.92 (s, 3H), 2.36 (d, J = 2.4 Hz, 3H), 2.16 (d, J = 3.2 Hz, 3H). | 467 |
| 81 | 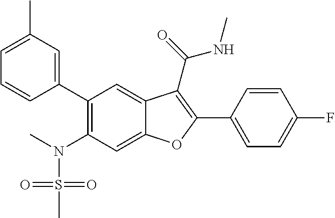 | 2-(4-fluorophenyl)- N-methyl-6-[methyl (methylsulfonyl) amino]-5-(3- methylphenyl)-1- benzofuran-3- carboxamide | 7.85~7.88 (m, 2H), 7.68 (s, 1H), 7.53 (s, 1H) 7.25 (t, J = 7.6 Hz, 1H), δ 7.10~7.18 (m, 5H), 5.79 (s, 1H), 3.07 (s, 3H), 2.91 (d, J = 4.8 Hz, 3H), 2.48 (s, 3H), 2.35 (s, 3H). | 467 |
| 82 | 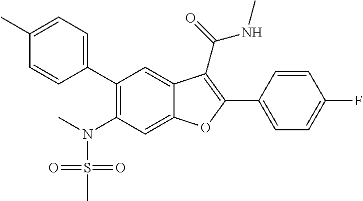 | 2-(4-fluorophenyl)- N-methyl-6-[methyl (methylsulfonyl) amino]-5-(4- methylphenyl)-1- benzofuran-3- carboxamide | 7.94~7.97 (m, 2H), 7.74 (s, 1H), 7.58 (s, 1H), 7.32~7.34 (m, 2H), 7.24~7.26 (m, 2H), 7.16~7.19 (m, 2H), 5.86 (s, 1H), 3.14 (s, 3H), 2.97~2.98 (d, J = 4.0 Hz, 3H), 2.58 (s, 3H), 2.41 (s, 3H). | 467 |
| 83 | 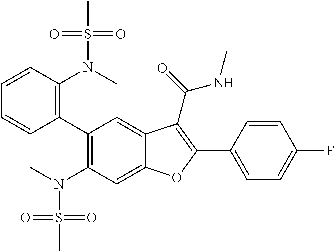 | 2-(4-fluorophenyl)- N-methyl-6-[methyl (methylsulfonyl) amino]-5-{2-[methyl (methylsulfonyl) amino]phenyl}-1- benzofuran-3- carboxamide | 7.99~8.01 (m, 2H), 7.98 (s, 1H), 7.50~7.55 (m, 2H), 7.41~7.49 (m, 3H), 7.22~7.26 (m, 2H), 3.23 (s, 6H), 3.07 (s, 3H), 2.93 (d, J = 4.0 Hz, 6H), 2.74 (s, 3H). | 560 |
| 84 | 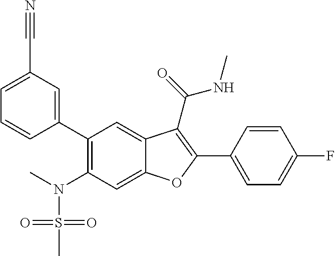 | 5-(3-cyanophenyl)-2- (4-fluorophenyl)-N- methyl-6-[methyl (methylsulfonyl) amino]-1- benzofuran-3- carboxamide | 7.83~7.87 (m, 2H), 7.75 (s, 1H), 7.65~7.68 (m, 3H), 7.63 (s, 1H), 7.48~7.54 (m, 1H), 7.13~7.17 (m, 2H), 5.75~5.76 (m, 1H), 3.09 (s, 3H), 2.92 (d, J = 4.0 Hz, 3H), 2.68 (s, 3H). | 478 |
| 85 | 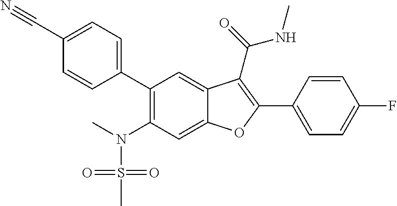 | 5-(4-cyanophenyl)-2- (4-fluorophenyl)-N- methyl-6-[methyl (methylsulfonyl) amino]-1- benzofuran-3- carboxamide | 7.82~7.85 (m, 2H), 7.76 (s, 1H), 7.66~7.68 (m, 2H), 7.51~7.53 (m, 3H), 7.13~7.17 (m, 2H), 5.65 (s, 1H), 3.07 (s, 3H), 2.92 (s, 3H), 2.70 (s, 3H). | 478 |
| 86 | 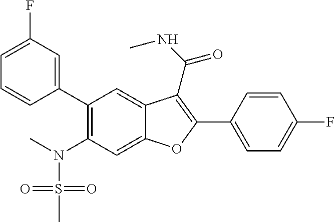 | 5-(3-fluorophenyl)-2- (4-fluorophenyl)-N- methyl-6-[methyl (methylsulfonyl) amino]-1- benzofuran-3- carboxamide | 7.94~7.98 (m, 2H), 7.83 (s, 1H), 7.64 (s, 1H), 7.42~7.47 (m, 1H), 7.10~7.26 (m, 5H), 5.87~5.88 (m, 1H), 3.19 (s, 3H), 3.03 (d, J = 5.2 Hz, 3H), 2.69 (s, 3H). | 471 |
| 87 | 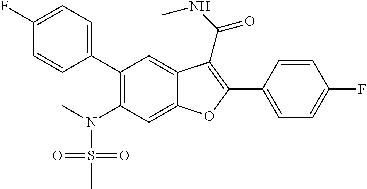 | 2,5-bis(4- fluorophenyl)-N- methyl-6-[methyl (methylsulfonyl) amino]-1- benzofuran-3- carboxamide | 7.84~7.88 (m, 2H), 7.71 (s, 1H), 7.33~7.37 (m, 2H), 7.05~7.19 (m, 5H), 5.75 (s, 1H), 3.05 (s, 3H), 2.91 (d, J = 4.0 Hz, 3H), 2.60 (s, 3H). | 471 |
| 88 | 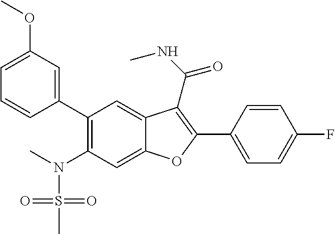 | 2-(4-fluorophenyl)-5- (3-methoxyphenyl)- N-methyl-6-[methyl (methylsulfonyl) amino]-1- benzofuran-3- carboxamide | 7.91~7.95 (m, 2H), 7.76 (s, 1H), 7.57 (s, 1H), 7.31~7.35 (m, 1H), 7.16~7.20 (m, 2H), 6.99~7.01 (m, 2H), 6.90~6.93 (m, 1H), 5.85 (s, 1H), 3.83 (s, 3H), 3.11 (s, 3H), 2.97~2.98 (d, J = 4.0 Hz, 3H), 2.62 (s, 3H). | 483 |
| 89 | 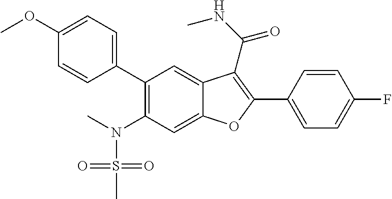 | 2-(4-fluorophenyl)-5- (4-methoxyphenyl)- N-methyl-6-[methyl (methylsulfonyl) amino]-1- benzofuran-3- carboxamide | 7.91~7.89 (m, 2H), 7.87 (s, 1H), 7.53 (s, 1H), 7.32~7.29 (m, 2H), 7.19~7.10 (m, 2H), 6.93~6.90 (m, 2H), 5.75 (s, 1H), 3.80 (s, 3H), 3.09 (s, 3H), 2.93 (s, 3H), 2.53 (s, 3H). | 483 |

### Example 90

### 5(2-fluorophenyl)-2-(4-fluorophenyl)-N-methoxy-6-[methyl(methylsulfonyl)amino]-1-benzofuran-3-carboxamide

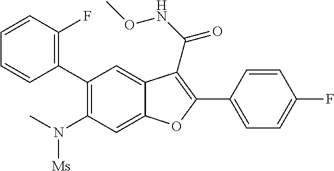

### Steps 1-5

Steps 1-5 were performed in accordance with Example 78, Steps 1-5.

### Step 6: 5-(2-fluorophenyl)-2-(4-fluorophenyl)-N-methoxy-6-[methyl(methylsulfonyl)amino]-1-benzofuran-3-carboxamide

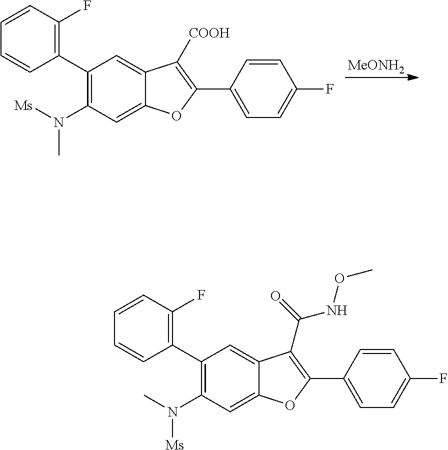

Example 90 was prepared using conditions analogous to the coupling reaction described in Example 7, Step 6 (40 mg, yield: 51%).

¹H-NMR (400 MHz, CDCl₃) δ 8.43 (s, 1H), 7.90˜7.93 (m, 2H), 7.74 (s, 1H), 7.62 (s, 1H), 7.36=7.38 (m, 2H), 7.13˜7.25 (m, 4H), 3.83 (s, 3H), 3.21 (s, 3H), 2.46 (s, 3H). MS (M+H)⁺: 487.

### Examples 91-98

Examples 91-98 were prepared according to the general procedures of Example 90.

| | | | | MS |
|---|---|---|---|---|
| Example | Structure | Name | ¹H-NMR (400 MHz, CDCl₃) δ | (M + H)⁺ |
| 91 | 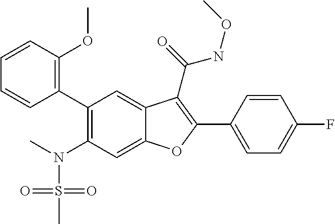 | 2-(4-fluorophenyl)-N- methoxy-5-(2- methoxyphenyl)-6- [methyl (methylsulfonyl)amino]- 1-benzofuran-3- carboxamide | 7.56 (s, 1H), 7.81~7.85 (m, 2H), 7.62 (s, 1H), 7.55 (s, 1H), 7.30~7.35 (m, 1H), 7.21~7.24 (m, 1H), 7.10~7.18 (m, 1H), 6.95~7.01 (m, 1H), 6.89~6.91 (d, J = 4.0 Hz, 1H), 3.80 (s, 3H), 3.70 (s, 3H), 3.11 (s, 3H), 2.34 (s, 3H). | 450 |
| 92 | 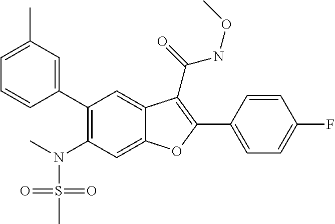 | 2-(4-fluorophenyl)-N- methoxy-6-[methyl (methylsulfonyl)amino]- 5-(3-methylphenyl)-1- benzofuran-3- carboxamide | 8.36 (s, 1H), 7.94~7.98 (m, 2H), 7.75 (s, 1H), 7.62 (s, 1H) 7.33 (t, J = 7.6 Hz, 1H), 7.18~7.26 (m, 5H), 3.87 (s, 3H), 3.15 (s, 3H), 2.53 (s, 3H), 2.42 (s, 3H). | 483 |
| 93 | 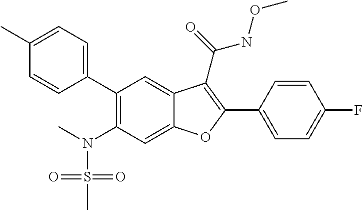 | 2-(4-fluorophenyl)-N- methoxy-6-[methyl (methylsulfonyl)amino]- 5-(4-methylphenyl)-1- benzofuran-3- carboxamide | 8.39 (s, 1H), 7.92~7.96 (m, 2H), 7.72 (s, 1H), 7.59 (s, 1H), 7.30~7.32 (m, 2H), 7.24~7.25 (m, 2H), 7.16~7.21 (m, 2H), 3.84 (s, 3H), 3.13 (s, 3H), 2.56 (s, 3H), 2.40 (s, 3H). | 483 |
| 94 | 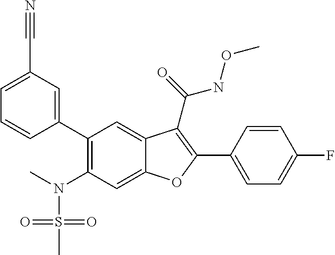 | 5-(3-cyanophenyl)-2- (4-fluorophenyl)-N- methoxy-6-[methyl (methylsulfonyl)amino]- 1-benzofuran-3- carboxamide | 8.37 (s, 1H), 7.84~7.88 (m, 2H), 7.72 (s, 1H), 7.62~7.68 (m, 3H), 7.56 (s, 1H), 7.48~7.54 (m, 1H), 7.13~7.17 (m, 2H), 3.80 (s, 3H), 3.09 (s, 3H), 2.68 (s, 3H). | 494 |
| 95 | 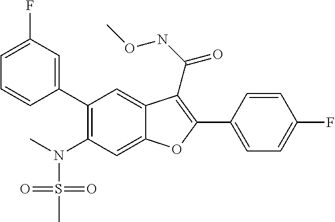 | 5-(3-fluorophenyl)-2- (4-fluorophenyl)-N- methoxy-6-[methyl (methylsulfonyl)amino]- 1-benzofuran-3- carboxamide | 8.29 (s, 1H), 7.91~7.95 (m, 2H), 7.76 (s, 1H), 7.61 (s, 1H), 7.38~7.44 (m, 1H), 7.07~7.23 (m, 5H), 3.85 (s, 3H), 3.14 (s, 3H), 2.63 (s, 3H). | 487 |
| 96 | 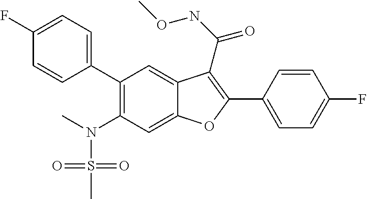 | 2,5-bis(4- fluorophenyl)-N- methoxy-6-[methyl (methylsulfonyl)amino]- 1-benzofuran-3- carboxamide | 8.25 (s, 1H), 7.85~7.89 (m, 2H), 7.69 (s, 1H), 7.54 (s, 1H), 7.33~7.37 (m, 2H), 7.06~7.17 (m, 4H), 3.80 (s, 3H), 3.07 (d, J = 4.0 Hz, 3H), 2.59 (s, 3H). | 487 |
| 97 | 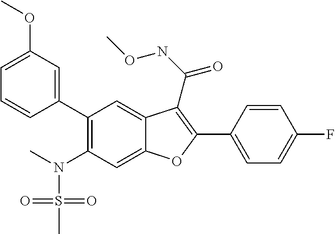 | 2-(4-fluorophenyl)-N- methoxy-5-(3- methoxyphenyl)-6- [methyl (methylsulfonyl)amino]- 1-benzofuran-3- carboxamide | 8.49 (s, 1H), 7.89~7.93 (m, 2H), 7.72 (s, 1H), 7.57 (s, 1H), 7.31~7.35 (m, 1H), 7.15~7.19 (m, 2H), 6.97~6.99 (m, 2H), 6.91~6.93 (m, 1H), 3.83 (s, 6H), 3.10 (s, 3H), 2.60 (s, 3H). | 499 |
| 98 | 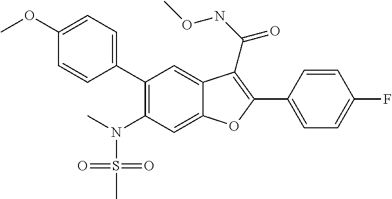 | 2-(4-fluorophenyl)-N- methoxy-5-(4- methoxyphenyl)-6- [methyl (methylsulfonyl)amino]- 1-benzofuran-3- carboxamide | 8.29 (s, 1H), 7.91~7.89 (m, 2H), 7.87 (s, 1H), 7.53 (s, 1H), 7.32~7.29 (m, 2H), 7.19~7.10 (m, 2H), 6.93~6.90 (m, 2H), 3.80 (s, 3H), 3.09 (s, 3H), 2.93 (s, 3H), 2.53 (s, 3H). | 499 |

### Example 99

### 2-(4-fluorophenyl)-N-methoxy-5-[3-(methoxycarbamoyl)phenyl]-6-[methyl(methylsulfonyl)amino]-1-benzofuran-3-carboxamide

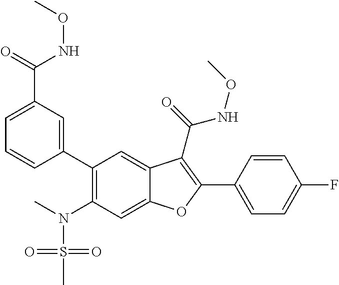

### Steps 1-4: ethyl 5-(3-cyanophenyl)-2-(4-fluorophenyl)-6-[methyl(methylsulfonyl)amino]-1-benzofuran-3-carboxylate

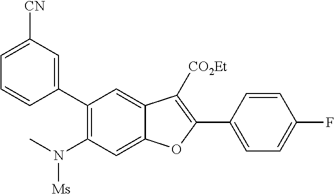

Steps 1-4 were performed in an analogous manner to Example 1, Steps 1-4.

### Step 5: 5-(3-cyanophenyl)-2-(4-fluorophenyl)-6-[methyl(methylsulfonyl)amino]-1-benzofuran-3-carboxylic acid and 5-(3-carboxyphenyl)-2-(4-fluorophenyl)-6-[methyl(methylsulfonyl)amino]-1-benzofuran-3-carboxylic acid

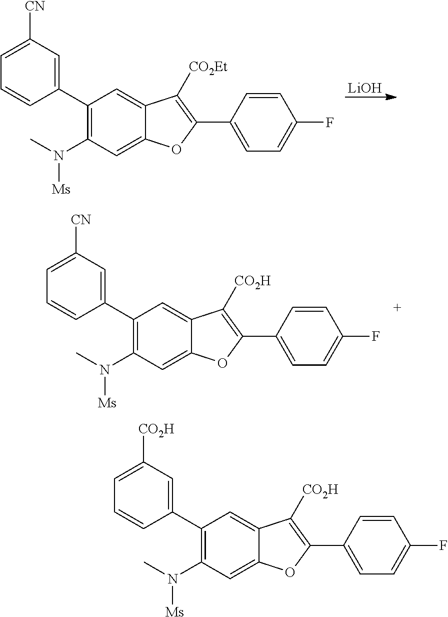

The ester (450 mg, 0.92 mmol) was dissolved in dioxane (5 mL). Then LiOH (96 mg, 4 mmol) was added to the solution, and the mixture was stirred at RT overnight. After acidifing with HCl (1 N) and extracting with EtOAc, the combined organic phases were washed with brine, dried over Na₂SO₄, filtered and evaporated to give the cyano carboxylic acid (300 mg, yield: 50%) and dicarboxylic acid (100 mg, yield: 30%). The crude mixture was used for the next step without further purification.

### Step 6: 2-(4-fluorophenyl)-N-methoxy-5-(3-(methoxycarbamoyl)phenyl]-6-[methyl(methylsulfonyl)amino]-1-benzofuran-3-carboxamide

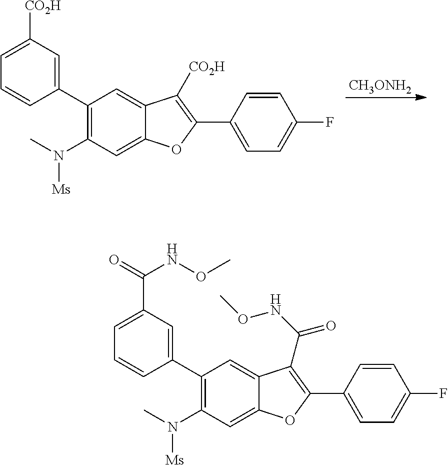

Example 99 was prepared using condition analogous to the coupling reaction described in Example 7, Step 6 (55 mg, yield: 73%).

¹H-NMR (400 MHz, CDCl₃) δ 9.49˜9.54 (m, 1H), 8.39 (s, 1H), 7.86˜7.89 (m; 2H), 7.83˜7.85 (m, 2H), 7.79 (s, 1H), 7.45˜7.51 (m, 3H), 7.13˜7.17 (m, 2H), 3.81˜3.82 (m, 6H), 2.99 (s, 3H), 2.78 (s, 3H). MS (M+H)⁺: 542.

### Example 100

### 2-(4-fluorophenyl)-N-methyl-5-[3-(methylcarbamoyl)phenyl]-6-[methyl(methylsulfonyl)amino]-1-benzofuran-3-carboxamide

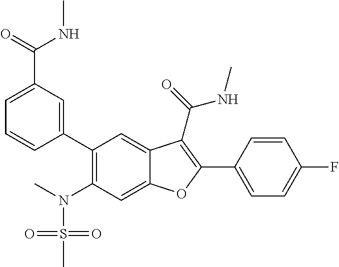

### Steps 1-5: 5-(3-carboxyphenyl)-2-(4-fluorophenyl)-6-[methyl(methylsulfonyl)amino]-1-benzofuran-3-carboxylic acid

Steps 1-5 were performed according to the general procedures in Example 99, Steps 1-5.

### Step 6: 2-(4-fluorophenyl)-N-methyl-5-[3-(methylcarbamoyl)phenyl]-6-[methyl(methylsulfonyl)amino]-1-benzofuran-3-carboxamide

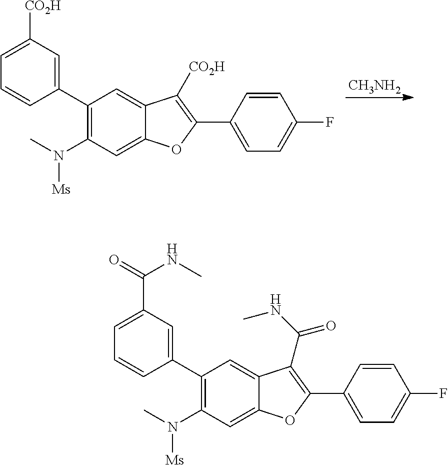

Example 100 was prepared according to the general procedure in Example 1, Step 6.

¹H-NMR (400 MHz, CDCl₃) δ 7.86˜7.89 (m, 2H), 7.78˜7.81 (m, 2H), 7.44˜7.51 (m, 3H), 7.12˜7.16 (t, J=12.0 Hz, 2H), 6.72˜6.73 (m, 1H), 5.81˜5.82 (m, 1H), 2.92˜2.95 (m, 6H), 2.90 (s, 3H), 2.84 (s, 3H). MS (M+H)⁺: 510.

### Example 101

### 5-[3-(aminomethyl)phenyl]-2-(4-fluorophenyl)-N-methyl-6-[methyl(methylsulfonyl)amino]-1-benzofuran-3-carboxamide

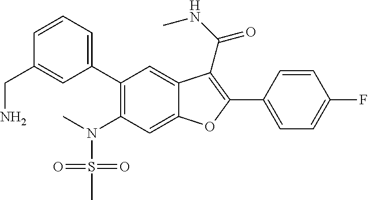

### Step 1: 5-[3-(aminomethyl)phenyl]-2-(4-fluorophenyl)-N-methyl-6-[methyl(methylsulfonyl)amino]-1-benzofuran-3-carboxamide

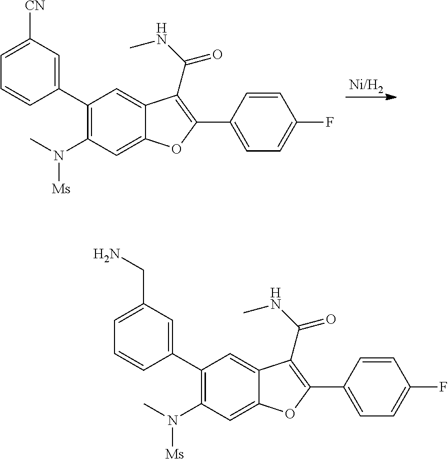

Raney-Ni (100 mg) and ammonia (conc. 0.5 mL) were added to a solution of the compound of Example 84 (58 mg, 0.13 mmol) in MeOH (20 mL). And then the mixture was degassed and stirred under 30 psi of H₂ overnight at RT. After filtered through CELITE, the filtrate was concentrated to give the desired benzylic amine (50 mg, yield: 85%).

¹H-NMR (400 MHz, CDCl₃) δ 7.79˜7.82 (m, 2H), 7.57 (s, 1H), 7.29 (d, J=8.0 Hz, 2H), 7.06˜7.10 (t, J=16.0 Hz, 2H), 6.58˜6.59 (m, 3H), 3.98 (s, 2H), 2.93 (s, 3H), 2.71 (d, J=4.0 Hz, 3H), 2.49 (s, 3H). MS (M+H)⁺: 482.

### Example 102

### 2-(4-fluorophenyl)-N-methyl-6-[methyl(methylsulfonyl)amino]-5-(3-{[(methylsulfonyl)amino]methyl}phenyl)-1-benzofuran-3-carboxamide

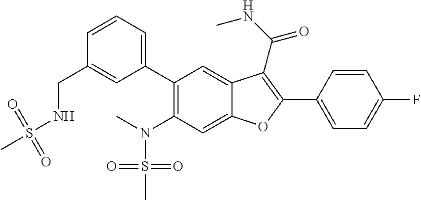

### Steps 1-2

Steps 1-2 were performed according to the general procedures in Example 1, Steps 1-2.

### Step 3: 2-(4-fluorophenyl)-N-methyl-6-[methyl(methylsulfonyl)amino]-5-(3-{[(methylsulfonyl)amino]methyl}phenyl)-1-benzofuran-3-carboxamide

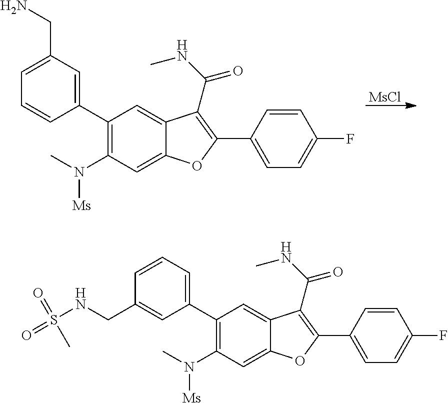

Example 102 was prepared in an analogous manner to the sulfonamide synthesis described in Example 1, Step 3 (20 mg, yield: 60%).

¹H-NMR (400 MHz, CDCl₃) δ 7.85˜7.88 (m, 2H), 7.76 (s, 1H), 7.52 (s, 1H), 7.46 (s, 1H), 7.36˜7.38 (m, 1H), 7.28˜7.32 (m, 2H), 7.12˜7.16 (m, 2H), 5.78˜5.79 (m, 1H), 4.95˜4.96 (m, 1H), 4.31 (d, J=8.0 Hz, 2H), 2.91˜2.93 (m, 6H), 2.86 (s, 3H), 2.79 (s, 3H). MS (M+H)⁺: 560.

### Example 103

Example 103 was prepared according to the general procedures of Example 102.

| | | | | MS |
|---|---|---|---|---|
| Example | Structure | Name | ¹H-NMR (400 MHz, CDCl₃) δ | (M + H)⁺ |
| 103 | 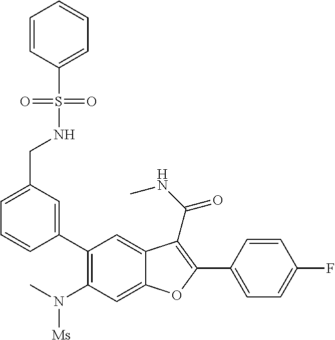 | 2-(4-fluorophenyl)-N- methyl-6- [methyl(methylsulfonyl) amino]-5-(3- {[(phenylsulfonyl)amino] methyl}phenyl)-1- benzofuran-3-carboxamide | 7.82~7.85 (m, 4H), 7.68 (s, 1H), 7.48~7.52 (m, 4H), 7.46 (s, 1H), 7.41~7.44 (m, 2H), 7.11~7.30 (m, 3H), 5.82~5.87 (m, 1H), 5.15~5.18 (m, 1H), 4.09~4.10 (m, 2H), 2.90~2.93 (m, 6H), 2.71 (s, 3H). | 622 |

### Example 104

### 2-(4-fluorophenyl)-N-methyl-6-dimethyl(methylsulfonyl)amino]-5-(4-{[(methylsulfonyl)amino]methyl}phenyl)-1-benzofuran-3-carboxamide

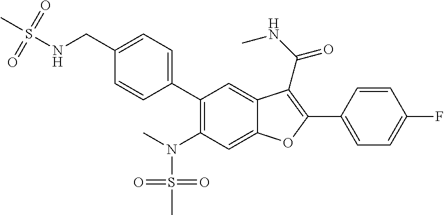

### Step 1: 5-[4-(aminomethyl)phenyl]-2-(4-fluorophenyl)-N-methyl-6-[methyl(methylsulfonyl)amino]-1-benzofuran-3-carboxamide

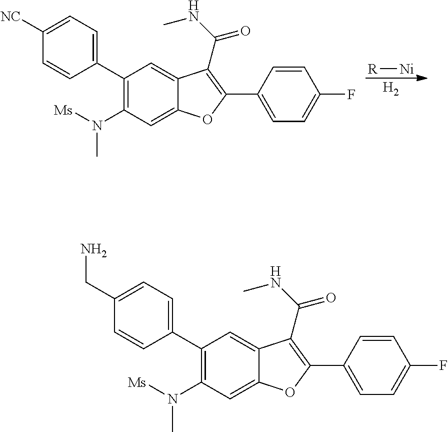

To a solution of the Compound of Example 85 (400 mg, 83.8 mmol) in MeOH (10 mL), and Raney-Ni (30 mg) was added. The reaction was degassed and then was shaken under 30 psi H₂ overnight. The reaction mixture was filtered, washed with MeOH. The solvent was evaporated to give the desired benzylic amine (350 mg, yield: 87%).

¹H-NMR (400 MHz, CDCl₃) 7.82˜7.85 (m, 2H), 7.47˜7.52 (m, 3H), 7.45 (s, 1H), 7.31˜7.37 (m, 2H), 6.99˜7.11 (m, 2H), 6.41 (s, 1H), 4.12 (s, 2H), 2.88 (s, 3H), 2.72 (d, J=4.0 Hz, 3H), 2.52 (s, 3H). MS (M+H)⁺: 482.

### Step 2: 2-(4-fluorophenyl)-N-methyl-6-[methyl(methylsulfonyl)amino]-5-(4-{[(methylsulfonyl)amino]methyl}phenyl)-1-benzofuran-3-carboxamide

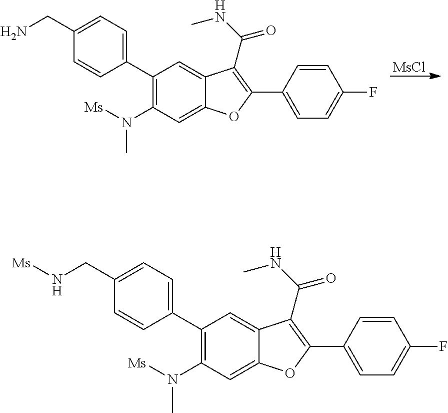

Example 104 was prepared in an analogous manner to the sulfonamide prepared in Example 1, Step 3 (20 mg, yield: 60%).

¹H-NMR (400 MHz, CDCl₃) 7.85˜7.88 (m, 2H), 7.69 (s, 1H), 7.51 (s, 1H), 7.37 (s, 4H), 7.14˜7.19 (m, 2H), 4.21 (s, 2H), 3.04 (s, 3H), 2.83 (s, 3H), 2.75 (s, 3H) 2.70 (s, 3H).

MS (M+H)⁺: 560.

### Examples 105-107

Examples 105-107 were prepared according to the general procedures of Example 104.

| Ex- | | | ¹H-NMR (400 MHz, | MS |
|---|---|---|---|---|
| ample | Structure | | CDCl₃) δ | (M + H)⁺ |
| 105 | 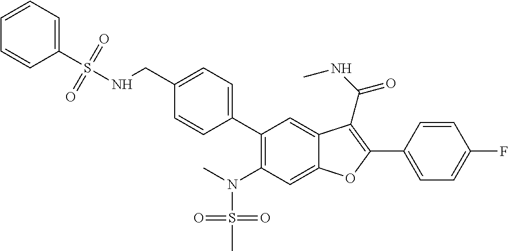 | 2-(4-fluorophenyl)-N- methyl-6-[methyl (methylsulfonyl)amino]- 5-(4- {[(phenylsulfonyl) amino]methyl}phenyl)- 1-benzofuran-3- carboxamide | 7.84~7.88 (m, 4H), 7.68 (s, 1H), 7.47~7.56 (m, 4H), 7.30~7.32 (m, 2H), 7.15~7.23 (m, 2H), 7.11~7.19 (m, 2H), 5.76 (s, 1H), 4.69 (s, 1H), 4.14 (d, J = 4.0 Hz, 2H), 3.04 (s, 3H), 2.88 (d, J = 8.0 Hz, 3H), 2.81 (s, 3H). | 622 |
| 106 | 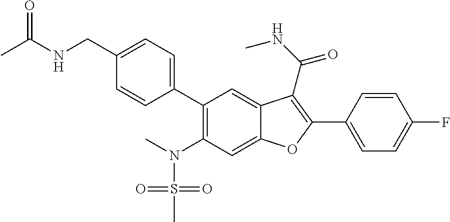 | 5-{4- [(acetylamino)methyl] phenyl}-2-(4- fluorophenyl)-N- methyl-6-[methyl (methylsulfonyl)amino]- 1-benzofuran-3- carboxamide | 7.84~7.87 (m, 2H), 7.66 (s, 1H), 7.492 (s, 1H), 7.32~7.34 (m, 2H), 7.25~7.27 (m, 2H), 7.09~7.13 (m, 2H), 5.94 (s, 1H), 4.39 (d, J = 8.0 Hz, 2H), 3.05 (s, 3H), 2.89 (d, J = 8.0 Hz, 3H), 2.57 (s, 3H), 1.96 (s, 3H). | 524 |
| 107 | 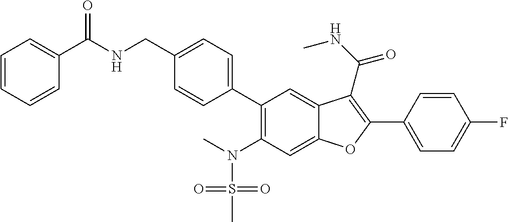 | 2-(4-fluorophenyl)-N- methyl-6-[methyl (methylsulfonyl)amino]- 5-(4- {[(phenylcarbonyl) amino]methyl}phenyl)- 1-benzofuran-3- carboxamide | 7.86~7.88 (m, 2H), 7.74~7.75 (m, 2H), 7.67 (s, 1H), 7.50 (s, 1H), 7.37~7.46 (m, 7H), 7.09~7.14 (m, 2H), 6.54 (s, 1H), 5.87 (s, 1H), 4.62 (d, J = 8.0 Hz, 2H), 3.06 (s, 3H), 2.89 (d, J = 4.0 Hz, 3H), 2.56 (s, 3H). | 586 |

### Example 108

### 2-(4-fluorophenyl)-N-methyl-6-[methyl(methylsulfonyl)amino]-5-[4-(trifluoromethyl)phenyl]-1-benzofuran-3-carboxamide

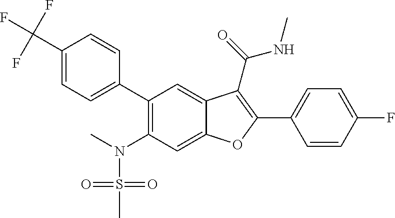

### Step 1: ethyl 2-(4-fluorophenyl)-6-nitro-1-benzofuran-3-carboxylate

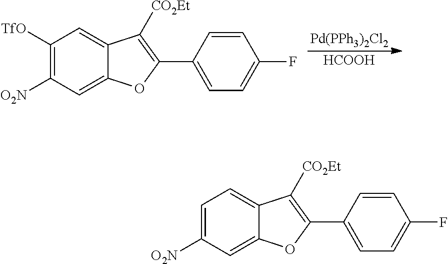

HCOOH (2.4 g, 71.23 mmol), Bu₃N (11.6 g, 85.47 mmol) and Pd(PPh₃)₂Cl₂ (197 mg, 0.28 mmol) were added to a solution of triflate (obtained according to procedure in WO 2004/041201 A2, 9 g, 28.49 mmol) in DMF (90 mL). The mixture was heated to 110° C. under N₂ protection. After stirred for 0.5 hour, the mixture was diluted with H₂O and extracted with ether. The combined organic layers were washed with brine, dried over Na₂SO₄, filtered and the solvent was evaporated. The crude product was purified by column to give pure nitro arene (4.78 g, yield: 51%).

¹H-NMR (400 MHz, CDCl₃) δ 8.36 (d, J=2 Hz, 1H), 8.20˜8.23 (m, 1H), 8.11 (d, J=8.8 Hz, 1H), 8.03˜8.07 (m, 2H), 7.13˜7.18 (m, 2H), 4.36˜4.41 (m, 2H), 1.37 (t, 1=7.2 Hz, 3H). MS (M+H)⁺: 330.

### Step 2: ethyl 6-amino-2-(4-fluorophenyl)-1-benzofuran-3-carboxylate

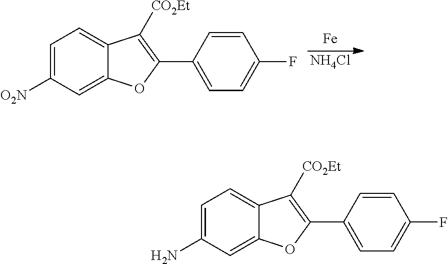

A mixture of the product of Step 1 (4.78 g, 14.5 mmol), Fe (4.06 g, 72.6 mmol) and NH₄Cl (6.20 g, 116 mmol) in H₂O/MeOH/THF (50 mL/50 mL/50 mL) was refluxed for 4 hours. Then, H₂O was added to quench the reaction, and the mixture was extracted with EtOAc. After washing with brine and dried, the solvent was removed by distillation. The pure aniline was obtained (3.47 g, yield: 80%) by prep-TLC.

¹H-NMR (400 MHz, CDCl₃) δ 7.94˜7.98 (m, 2H), 7.73 (d, J=8 Hz, 1H), 7.08 (t, J=8.8 Hz, 2H), 6.77 (s, 1H), 6.68 (d, J=6.8 Hz, 1H), 4.30˜4.35 (m, 2H), 1.34 (t, J=7.2 Hz, 3H). MS (M+H)⁺: 300.

### Step 3: ethyl 2-(4-fluorophenyl)-6-[(methylsulfonyl)amino]-1-benzofuran-3-carboxylate

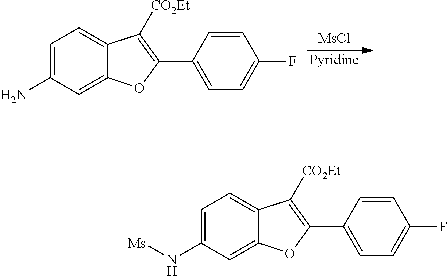

MsCl (122 mg, 1.06 mmol) was added to a solution of aniline (200 mg, 0.67 mmol) and pyridine (107 mg, 1.35 mmol) in dry DCM (2 mL). After stirred overnight at RT, the mixture was diluted with H₂O and extracted with DCM. The organic layer was washed with brine, dried over Na₂SO₄ and filtered, and the solvent was evaporated. The crude product was purified by prep-TLC to give the desired sulfonamide (200 mg, yield: 78.5%).

¹H-NMR (400 MHz, CDCl₃) δ 7.97˜8.06 (m, 3H), 7.53˜7.54 (m, 1H), 7.11˜7.19 (m, 3H), 6.74 (s, 1H), 4.30˜4.35 (m, 2H), 3.93 (s, 3H), 1.34 (t, J=7.2 Hz, 3H). MS (M+H)⁺: 378.

### Step 4: ethyl 2-(4-fluorophenyl)-6-[methyl(methylsulfonyl)amino]-1-benzofuran-3-carboxylate

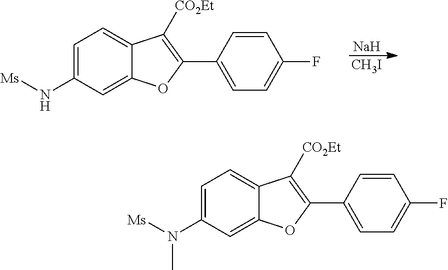

NaH (60% in oil, 111 mg, 2.78 mmol) and CH₃I (395 mg, 2.78 mmol) were added to a solution of sulfonamide (211 mg, 0.56 mmol) in dry DMF (4 mL) under N₂. After stirred overnight at RT, ice cold diluted AcOH was added, and the mixture was extracted with EtOAc. The organic layer was washed with brine, dried over Na₂SO₄ and filtered, and the solvent was evaporated under reduced pressure. The crude product was used for the next step without further purification (210 mg, yield: 96%).

### Step 5: ethyl 5-bromo-2-(4-fluorophenyl)-6-[methyl(methylsulfonyl)amino]-1-benzofuran-3-carboxylate

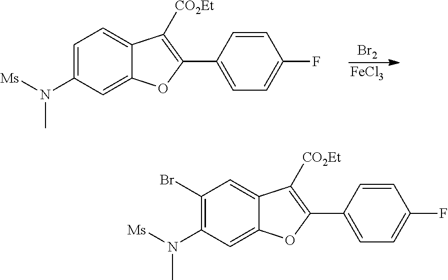

A stirred solution of sulfonamide (500 mg, 1.3 mmol) and FeCl₃ (210 mg, 0.78 mmol) in dry CCl₄ (5 mL) was added Br₂ (210 mg, 1.3 mmol) in dry CCl₄ (2 mL). The mixture was allowed to stir at 50° C. for 4 hours. The mixture was cooled, diluted with H₂O, and extracted with DCM; the organic solvent was washed with brine, dried over Na₂SO₄ and filtered; and the solvent was evaporated under reduced pressure. The crude was purified by column chromatography to give aryl bromide (240 mg, yield: 30%).

¹H-NMR (400 MHz, CDCl₃) δ 8.25 (s, 1H), 7.91˜8.05 (m, 2H), 7.62 (s, 1H), 7.02˜7.15 (m, 2H), 4.32˜4.46 (m, 2H), 3.37 (s, 3H), 3.02 (s, 3H), 1.35 (t, J=4.4 Hz, 3H). MS (M+H)⁺: 470.

### Step 6: 5-bromo-2-(4-fluorophenyl)-6-[methyl(methylsulfonyl)amino]-1-benzofuran-3-carboxylic acid

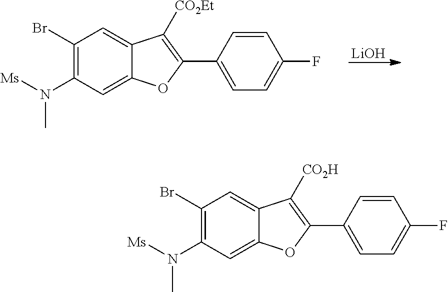

The ester (210 mg, yield: 80%) was hydrolysed in an analogous manner to the general procedure of Example 78, Step 5. The carboxylic acid was used in the next step without further purification.

### Step 7: 5-bromo-2-(4-fluorophenyl)-N-methyl-6-[methyl(methylsulfonyl)amino]-1-benzofuran-3-carboxamide

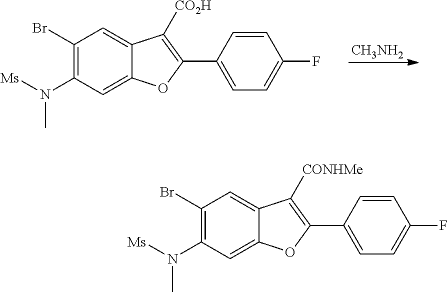

The amide was prepared according to the general procedure in Example 1, Step 6 (180 mg, yield: 75%).

¹H-NMR (400 MHz, CDCl₃) δ 8.09 (s, 1H), 7.81˜7.85 (m, 2H), 7.63 (s, 1H), 7.12˜7.19 (m, 2H), 5.71 (br, 1H), 3.27 (s, 3H), 3.02 (s, 3H), 2.93 (d, J=4.4 Hz, 3H). MS (M+H)⁺: 455.

### Step 8: 2-(4-fluorophenyl)-N-methyl-64-methyl(methylsulfonyl)amino]-5-[4-(trifluoromethyl)phenyl]-1-benzofuran-3-carboxamide

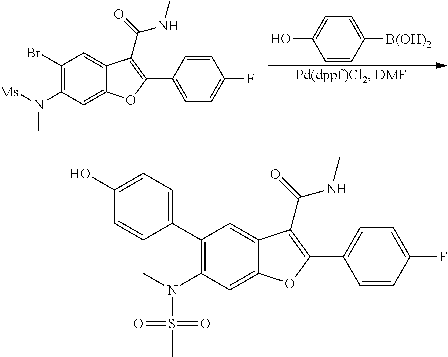

To a solution of 5-bromo-2-(4-fluorophenyl)-N-methyl-6-[methyl(methylsulfonyl)amino]-1-benzofuran-3-carboxamide (30 mg, 0.066 mmol) in DMF (2 mL) were added 4-hydroxy-phenyl boronic acid (21 mg, 0.13 mmol) and K₃PO₄.3H₂O (36.5 mg, 0.13 mmol). Then, Pd(dppf)Cl₂ (3.4 mg, 0.004 mmol) was added under N₂. The resulting mixture was heated to 90° C. for 12 hours. The mixture was cooled to RT, then filtered and purified by prep-HPLC to give 2-(4-fluorophenyl)-5-(4-hydroxyphenyl)-N-methyl-6-[methyl(methylsulfonyl)amino]-1-benzofuran-3-carboxamide. (4.8 mg, Yield; 15.5%).

MS (M+H)⁺: 469.

### Examples 109-122

Examples 109-122 were prepared according to the general procedures of Example 108.

| | | | MS |
|---|---|---|---|
| Example | Structure | Name | (M + H)⁺ |
| 109 | 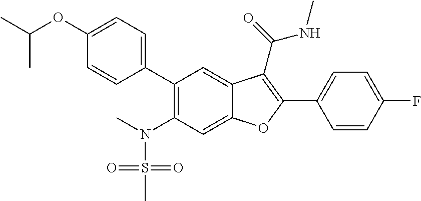 | 2-(4-fluorophenyl)-N-methyl-6- [methyl(methylsulfonyl)amino]-5- [4-(propan-2-yloxy)phenyl]-1- benzofuran-3-carboxamide | 511 |
| 110 | 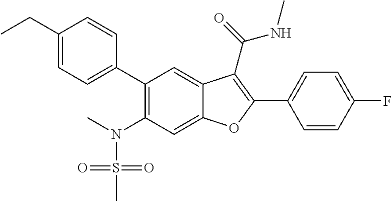 | 5-(4-ethylphenyl)-2-(4- fluorophenyl)-N-methyl-6- [methyl(methylsulfonyl)amino]-1- benzofuran-3-carboxamide | 481 |
| 111 | 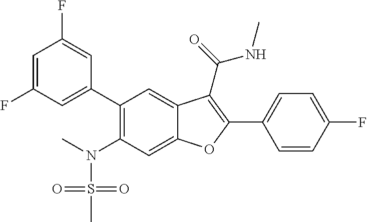 | 5-(3,5-difluorophenyl)-2-(4- fluorophenyl)-N-methyl-6- [methyl(methylsulfonyl)amino]-1- benzofuran-3-carboxamide | 489 |
| 112 | 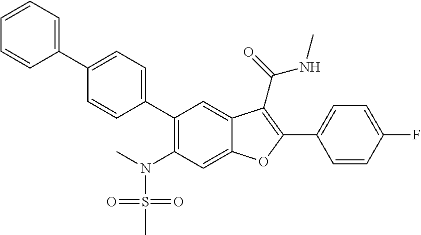 | 5-(biphenyl-4-yl)-2-(4- fluorophenyl)-N-methyl-6- [methyl(methylsulfonyl)amino]-1- benzofuran-3-carboxamide | 529 |
| 113 | 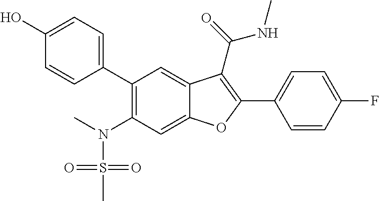 | 2-(4-fluorophenyl)-5-(4- hydroxyphenyl)-N-methyl-6- [methyl(methylsulfonyl)amino]-1- benzofuran-3-carboxamide | 469 |
| 114 | 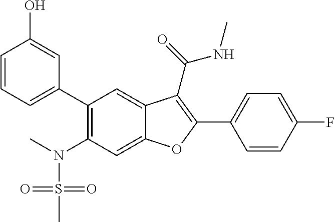 | 2-(4-fluorophenyl)-5-(3- hydroxyphenyl)-N-methyl-6- [methyl(methylsulfonyl)amino]-1- benzofuran-3-carboxamide | 469 |
| 115 | 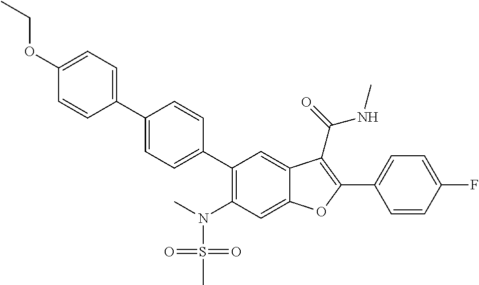 | 5-(4′-ethoxybiphenyl-4-yl)-2-(4- fluorophenyl)-N-methyl-6- [methyl(methylsulfonyl)amino]- 1-benzofuran-3-carboxamide | 573 |
| 118 | 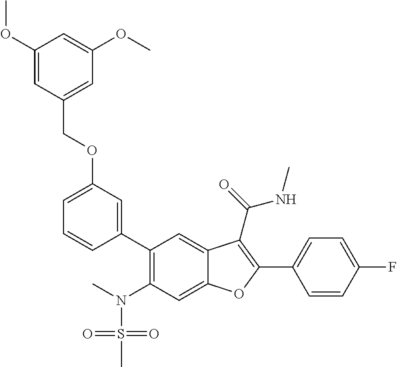 | 5-{3-[(3,5- dimethoxybenzyl)oxy]phenyl}-2- (4-fluorophenyl)-N-methyl-6- [methyl(methylsulfonyl)amino]-1- benzofuran-3-carboxamide | 619 |
| 119 | 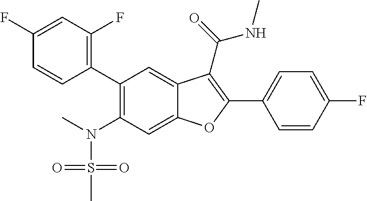 | 5-(2,4-difluorophenyl)-2-(4- fluorophenyl)-N-methyl-6- [methyl(methylsulfonyl)amino]-1- benzofuran-3-carboxamide | 489 |
| 121 | 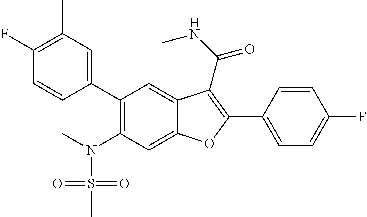 | 5-(4-fluoro-3-methylphenyl)-2-(4- fluorophenyl)-N-methyl-6- [methyl(methylsulfonyl)amino]-1- benzofuran-3-carboxamide | 485 |
| 122 | 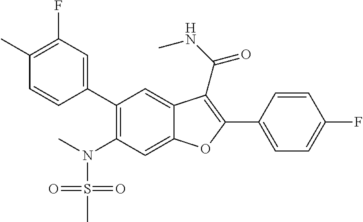 | 5-(3-fluoro-4-methylphenyl)-2-(4- fluorophenyl)-N-methyl-6- [methyl(methylsulfonyl)amino]-1- benzofuran-3-carboxamide | 485 |

### Example 123

### 6-{[2-(benzylamino)ethyl](methylsulfonyl)amino}-2-(4-fluorophenyl)-N-methyl-5-phenyl-1-benzofuran-3-carboxamide

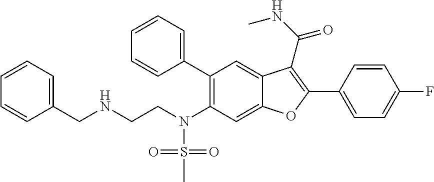

### Steps 1-4

Steps 1-4 were performed in an analogous manner to Example 1, Steps 1-4.

### Step 5: ethyl 2-(4-fluorophenyl)-6-[(methylsulfonyl){2-[(methylsulfonyl]oxy)ethyl}amino]-5-phenyl-1-benzofuran-3-carboxylate

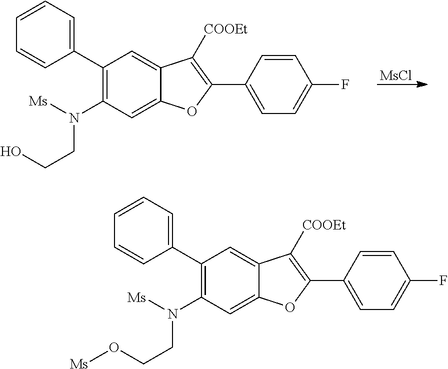

MsCl (0.2 mL, 3.0 mmol) was added to a solution of alcohol (1 g, 2.0 mmol) and Et₃N (0.6 mL, 4.0 mmol) in dry DCM (10 mL), in a manner similar to that of Example 1, Step 4. The reaction mixture was stirred overnight at RT. After dilution with H₂O and extraction with DCM, the mixture was washed with brine, dried over Na₂SO₄ and filtered, and the solvent was evaporated under reduced pressure. The crude product was purified by column to give the mesylate (800 mg, yield: 75%).

¹H-NMR (400 MHz, CDCl₃) δ 8.00˜8.03 (m, 2H), 7.99 (s, 1H), 7.57 (s, 1H), 7.48˜7.50 (m, 2H), 7.35˜7.43 (m, 3H), 7.11˜7.16 (m, 2H), 4.30˜4.35 (dd, J=8.0 Hz, 2H), 4.02˜4.05 (m, 2H), 3.21˜3.83 (m, 2H), 2.98 (s, 3H), 2.90 (s, 3H), 1.27˜1.30 (m, 3H). MS (M+H)⁺: 576.

### Step 6: ethyl 6-{[2-(benzylamino)ethyl](methylsulfonyl)amino}-2-(4-fluorophenyl)-5-phenyl-1-benzofuran-3-carboxylate

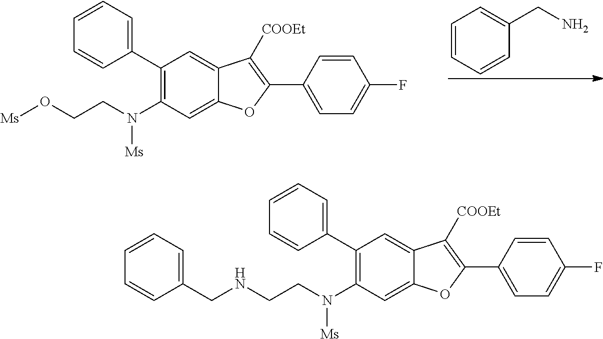

Benzylamine (0.5 mL, 0.27 mmol)) was added to a solution of mesylate (50 mg, 0.09 mmol) in Et₃N (1 mL) and MeCN (1 mL). The reaction mixture was stirred overnight at 60° C. After dilution with H₂O and extraction with EtOAc, the mixture was washed with brine, dried over Na₂SO₄ and filtered, and the solvent was evaporated under reduced pressure. The crude product was purified by prep-TLC to give the benzylic amine (30 mg, yield: 58%).

¹H-NMR (400 MHz, CDCl₃) δ 8.00˜8.03 (m, 2H), 7.99 (s, 1H), 7.57 (s, 1H), 7.48˜7.50 (m, 2H), 7.35˜7.43 (m, 7H), 7.11˜7.16 (m, 3H), 4.30˜4.35 (dd, J=8.0 Hz, 2H), 4.02˜4.05 (m, 2H), 3.21˜3.83 (m, 2H), 2.98 (s, 3H), 2.32 (d, J=8.0 Hz, 2H), 1.27˜1.30 (m, 3H).

MS (M+H)⁺: 587.

### Step 7: 6-{[2-(benzylamino)ethyl](methylsulfonyl)amino}-2-(4-fluorophenyl)-5-phenyl-1-benzofuran-3-carboxylic acid

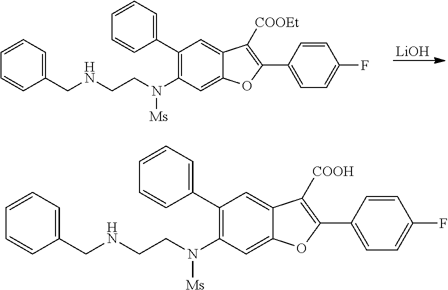

The ester (30 mg, 0.05 mmol) was dissolved in 1,4-dioxane (1 mL) and H₂O (1 mL). Then LiOH (21 mg, 0.5 mmol) was added to the solution, and the mixture was refluxed for 2 hours. After being acidified with HCl (1 N) and extracted with EtOAc, the combined organic phases were washed with brine, dried over Na₂SO₄, filtered and evaporated to give the carboxylic acid (22 mg, yield: 79%). The acid was used in the next step without further purification.

### Step 8: 6-{[2-(benzylamino)ethyl](methylsulfonyl)amino}-2-(4-fluorophenyl)-N-methyl-5-phenyl-1-benzofuran-3-carboxamide

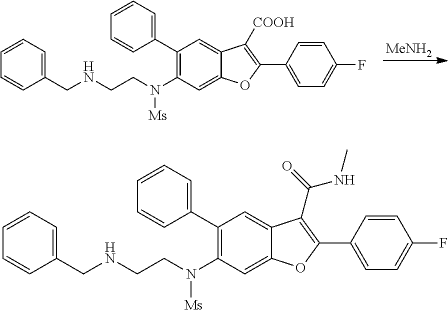

Carboxylic acid (22 mg, 0.04 mmol), HOBT (10 mg, 0.06 mmol) and EDCI (19 mg, 0.10 mmol) were dissolved in dry DMF (1 mL). The resulting solution was stirred for 30 minutes. Then, methanamine HCl salt (11 mg, 0.16 mmol) and Et₃N (18 mg, 0.18 mmol) was added to the mixture. After stirred overnight, the mixture was diluted with H₂O and extracted with EtOAc. The combined organic phases were washed with brine, dried over Na₂SO₄, filtered and evaporated. The crude product was purified by prep-HPLC to give pure amide (Example 124) (20 mg, yield: 70%).

¹H-NMR (400 MHz, CDCl₃) δ 7.87˜7.88 (m, 2H), 7.68 (s, 1H), 7.44 (s, 1H), 7.38˜7.42 (m, 2H), 7.23˜7.25 (m, 6H), 7.13˜7.19 (m, 4H), 5.87 (s, 1H), 3.58˜3.61 (m, 2H), 3.51˜3.52 (m, 2H), 3.06 (s, 3H), 2.91 (s, 3H), 2.53˜2.59 (m, 2H). MS (M+H)⁺: 572.

### Examples 124-132

Examples 124-132 were prepared according to the general procedures of Example 123.

| Ex- | | | | MS |
|---|---|---|---|---|
| ample | Structure | Name | ¹H-NMR (400 MHz, CDCl₃) δ | (M + H)⁺ |
| 124 | 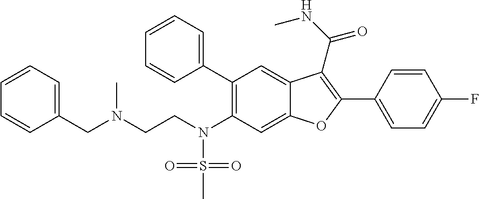 | 6-[{2- [benzyl(methyl) amino]ethyl} (methylsulfonyl) amino]-2-(4- fluorophenyl)-N- methyl-5-phenyl-1- benzofuran-3- carboxamide | 7.88~7.91 (m, 2H), 7.73 (s, 1H), 7.45 (s, 1H), 7.38~7.42 (m, 2H), 7.34~7.36 (m, 6H), 7.12~7.17 (m, 4H), 5.84 (s, 1H), 3.70~3.81 (m, 4H), 2.88~2.93 (m, 8H), 2.43 (s, 3H). | 586 |
| 125 | 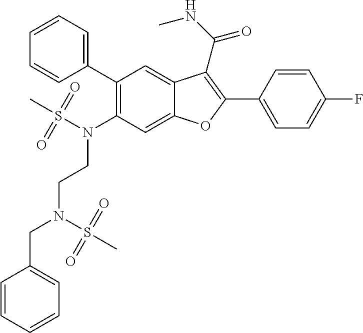 | 6-[{2-[benzyl (methylsulfonyl) amino]ethyl} (methylsulfonyl) amino]-2-(4- fluorophenyl)-N- methyl-5-phenyl-1- benzofuran-3- carboxamide | 7.86~7.89 (m, 2H), 7.65 (s, 1H), 7.41 (s, 1H), 7.31~7.34 (m, 5H), 7.19~7.21 (m, 3H), 7.12~7.16 (m, 4H), 5.84 (s, 1H), 4.16~4.18 (m, 2H), 3.28~3.33 (m, 2H), 3.11~3.14 (m, 2H), 2.95 (s, 3H), 2.66 (s, 3H), 2.63 (s, 3H). | 650 |
| 126 | 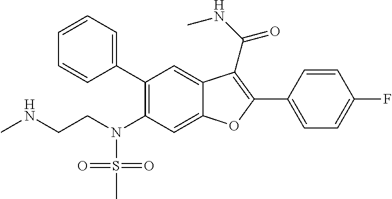 | 2-(4-fluorophenyl)- N-methyl-6-{[2- (methylamino)ethyl] (methylsulfonyl) amino}-5-phenyl-1- benzofuran-3- carboxamide | 7.78~7.92 (m, 2H), 7.70 (s, 1H), 7.57 (s, 1H), 7.41~7.55 (m, 4H), 7.35~7.39 (m, 1H), 7.13~7.16 (m, 2H), 6.06 (s, 1H), 3.43~3.52 (m, 2H), 3.24~3.27 (m, 2H), 2.95 (s, 3H), 2.86 (s, 3H), 2.76 (s, 3H). | 496 |
| 127 | 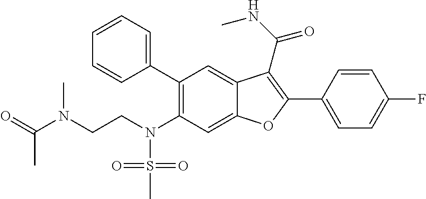 | 6-[{2-[acetyl(methyl) amino]ethyl} (methylsulfonyl) amino]-2-(4- fluorophenyl)-N- methyl-5-phenyl-1- benzofuran-3- carboxamide | 7.95~7.98 (m, 2H), 7.84 (s, 1H), 7.76 (s, 1H), 7.43~7.46 (m, 5H), 7.18~7.22 (m, 2H), 5.83 (s, 1H), 3.56~3.65 (m, 2H), 3.49~3.52 (m, 2H), 2.98 (s, 3H), 2.95 (s, 3H), 2.88 (s, 3H), 1.97 (s, 3H). | 538 |
| 128 | 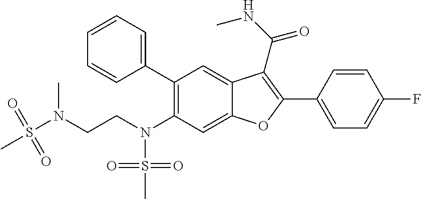 | 2-(4-fluorophenyl)- N-methyl-6-[{2- [methyl (methylsulfonyl) amino]ethyl} (methylsulfonyl) amino]-5-phenyl-1- benzofuran-3- carboxamide | 7.95~7.99 (m, 2H), 7.79 (s, 1H), 7.75 (s, 1H), 7.57~7.59 (m, 2H), 7.44~7.47 (m, 3H), 7.17~7.22 (m, 2H), 5.84 (s, 1H), 3.76~3.80 (m, 2H), 3.30~3.34 (m, 2H), 3.14 (s, 3H), 3.09 (s, 3H), 2.84 (s, 3H), 2.73 (s, 3H). | 574 |
| 129 | 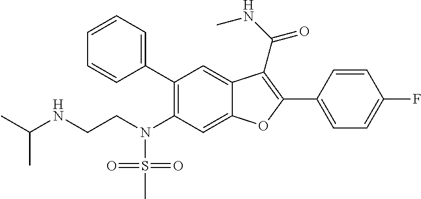 | 2-(4-fluorophenyl)- N-methyl-6- {(methylsulfonyl)[2- (propan-2- ylamino)ethyl] amino}-5-phenyl-1- benzofuran-3- carboxamide | 7.95~8.10 (m, 2H), 7.91 (s, 1H), 7.36~7.40 (m, 3H), 7.15~7.26 (m, 3H), 6.96~7.01 (m, 2H), 5.95 (s, 1H), 3.99~4.03 (m, 2H), 3.05 (s, 3H), 2.93 (s, 3H), 2.84~2.86 (m, 2H), 2.59~2.60 (m, 1H), 1.17~1.17 (m, 6H). | 524 |
| 130 | 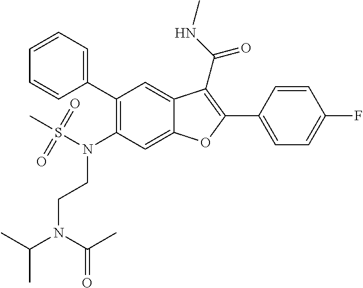 | 6-[{2-[acetyl (propan-2-yl) amino]ethyl} (methylsulfonyl) amino]-2-(4- fluorophenyl)-N- methyl-5-phenyl-1- benzofuran-3- carboxamide | 8.05~8.09 (m, 2H), 8.04 (s, 1H), 7.61 (s, 1H), 7.37~7.49 (m, 5H), 7.17~7.21 (m, 2H), 6.03 (s, 1H), 3.85~3.91 (m, 1H), 3.41~3.46 (m, 2H), 3.22~3.29 (m, 2H), 3.13 (s, 3H), 2.87 (s, 3H), 2.03 (s, 3H), 0.87~1.06 (m, 6H). | 566 |
| 131 | 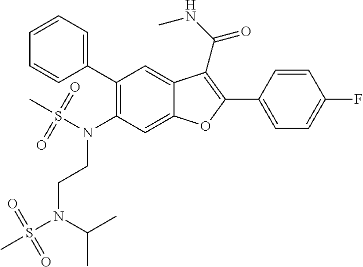 | 2-(4-fluorophenyl)- N-methyl-6- [(methylsulfonyl){2- [(methylsulfonyl) (propan-2- yl)amino]ethyl} amino]-5-phenyl-1- benzofuran-3- carboxamide | 7.94~7.98 (m, 2H), 7.80 (s, 1H), 7.59 (s, 1H), 7.50~7.52 (m, 2H), 7.42~7.47 (m, 3H), 7.18~7.23 (m, 2H), 5.85 (s, 1H), 3.91~3.98 (m, 1H), 3.48~3.54 (m, 2H), 2.98~2.99 (m, 2H), 2.84 (s, 3H), 2.72 (s, 3H), 2.61 (s, 3H), 1.04~1.06 (m, 6H). | 602 |
| 132 | 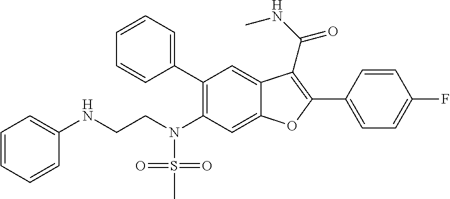 | 2-(4-fluorophenyl)- N-methyl-6- amethylsulfonyl)[2- (phenylamino)ethyl] amino}-5-phenyl-1- benzofuran-3- carboxamide | 7.90~7.86 (m, 2H), 7.75 (s, 1H), 7.57 (s, 1H), 7.50~7.48 (m, 2H), 7.43~7.42 (m, 2H), 7.40~7.38 (m, 3H), 7.16~7.07 (m, 2H), 6.71~6.37 (m, 1H), 6.39~6.37 (m, 2H), 5.79 (s, 1H), 3.55~3.50 (m, 2H), 3.13~3.12 (m, 1H), 2.94 (s, 3H), 2.93~2.92 (m, 1H), 2.71 (s, 3H). | 558 |

### Example 133

### 2-(4-fluorophenyl)-6-[(2-hydroxyethyl)(methylsulfonyl)amino]-N-methyl-5-phenyl-1-benzofuran-3-carboxamide

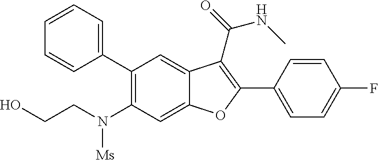

### Steps 1-3

Steps 1-3 were performed in accordance with Example 1, Steps 1-3.

### Step 4: ethyl 2-(4-fluorophenyl)-6-[(2-hydroxyethyl)(methylsulfonyl)amino]-5-phenyl-1-benzofuran-3-carboxylate

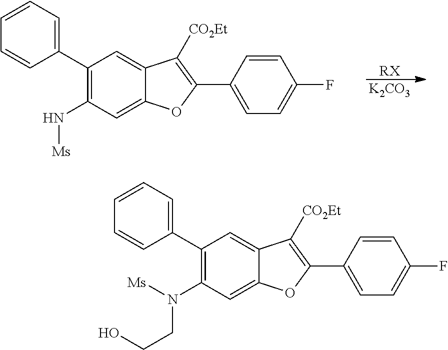

KI (6 mg, 0.036 mmol), K₂CO₃ (46 mg, 0.33 mmol), and 2-bromo ethanol (80 mg, 0.563 mmol) were added to a solution of ethyl 2-(4-fluorophenyl)-6-[(methylsulfonyl)amino]-5-phenyl-1-benzofuran-3-carboxylate (50 mg, 0.131 mmol) in dry DMF under N₂ protection. The mixture was stirred at 60° C. overnight. After dilution with H₂O and extraction with EtOAc, the organic solvent was washed with brine, dried over Na₂SO₄ and filtered, and the solvent was evaporated under reduced pressure. The crude was purified by prep-TLC to give the desired product of ethyl 2-(4-fluorophenyl)-6-[(2-hydroxyethyl) (methylsulfonyl)amino]-5-phenyl-1-benzofuran-3-carboxylate (60 mg, yield: 91%).

¹H-NMR (400 MHz, CDCl₃) δ 8.00˜8.03 (m, 3H), 7.61 (s, 1H), 7.50-7.52 (2H), 7.35˜7.44 (m, 3H), 7.11˜7.16 (m, 2H), 4.30˜4.36 (m, 2H), 3.21˜3.56 (m, 4H), 2.91 (s, 3H), 1.29 (t, J=7.2 Hz, 3H).

### Step 5: 2-(4-fluorophenyl)-6-[(2-hydroxyethyl)(methylsulfonyl)amino]-5-phenyl-1-benzofuran-3-carboxylic acid

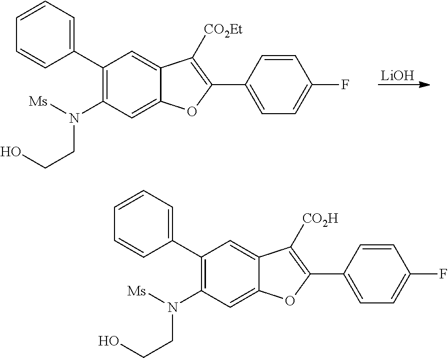

To a solution of the product of Step 4 (60 mg, 0.12 mmol) in dioxane (1 mL) was added LiOH.H₂O (40 mg, 0.952 mmol) and H₂O (1 mL), and the resultant solution was stirred for 2 hours at 60° C. H₂O was added, and then 2N aqueous HCl was added to adjust pH=4˜5. After extraction with EtOAc, the combined organic layer was washed with brine, dried over Na₂SO₄, and evaporated to provide the crude product. The crude was purified by prep-TLC. The desired product of 2-(4-fluorophenyl)-6-[(2-hydroxyethyl)(methylsulfonyl)amino]-5-phenyl-1-benzofuran-3-carboxylic acid was obtained (50 mg, yield: 88%).

¹H-NMR (400 MHz, CDCl₃) δ 8.05 (s, 1H), 7.99˜8.03 (m, 2H), 7.62 (s, 1H), 7.48˜7.49 (m, 2H), 7.38˜7.43 (m, 3H), 7.11˜7.15 (m, 2H), 3.19˜3.59 (m, 4H), 2.90 (s, 3H).

### Step 6: 2-(4-fluorophenyl)-6-[(2-hydroxyethyl)(methylsulfonyl)amino]-N-methyl-5-phenyl-1-benzofuran-3-carboxamide

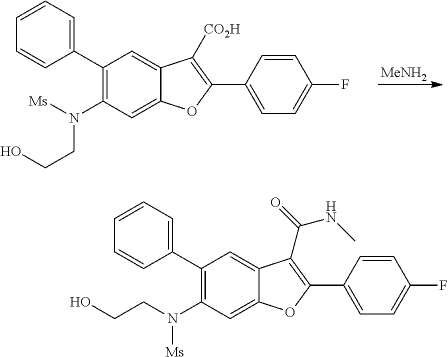

The product of Step 5 (20 mg, 0.043 mmol), HOBT (12 mg, 0.08 mmol) and EDCI (26 mg, 0.13 mmol) were dissolved in dry DMF (1 mL). The resulting solution was stirred for 30 minutes. Then, methanamine (HCl salt, 7 mg, 0.22 mmol) and Et₃N (25 mg, 0.24 mmol) were added to the mixture. After stirring overnight, the mixture was diluted with H₂O and extracted with EtOAc. The combined organic phases were washed with brine, dried over Na₂SO₄, filtered and evaporated. The crude product was purified by prep-TLC to give pure 2-(4-fluorophenyl)-6-[(2-hydroxyethyl)(methylsulfonyl)amino]-N-methyl-5-phenyl-1-benzofuran-3-carboxamide (10 mg, yield: 48%).

¹H-NMR (400 MHz, CDCl₃) δ 7.86˜7.90 (m, 2H), 7.72 (s, 1H), 7.59 (s, 1H), 7.47˜7.50 (m, 2H), 7.32˜7.40 (m, 3H), 7.10˜7.16 (m, 2H), 5.80 (s, 1H), 3.28˜3.47 (m, 4H), 2.90 (s, 6H).

### Example 134

### 2-(4-fluorophenyl)-N-methyl-6-[{2-[methyl(phenyl)amino]ethyl}(methylsulfonyl)amino]-5-phenyl-1-benzofuran-3-carboxamide

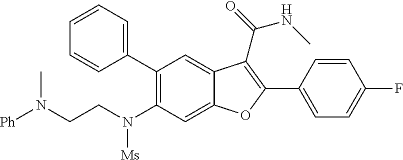

### Steps 1-3

Steps 1-3 were performed in accordance with Example 1, Steps 1-3.

### Step 4: ethyl 2-(4-fluorophenyl)-6-[{2-[methyl(phenyl)amino]ethyl}(methylsulfonyl)amino]-5-phenyl-1-benzofuran-3-carboxylate

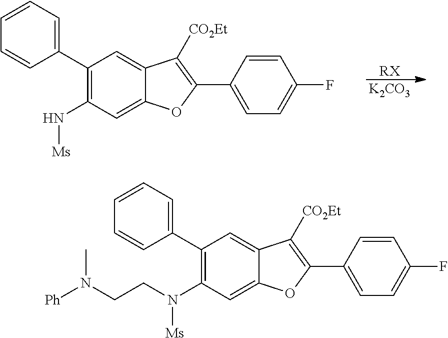

Step 4 was performed in an analogous manner to Example 133, Step 4. The crude product was purified by prep-TLC to give pure ethyl 2-(4-fluorophenyl)-6-[{2-[methyl(phenyl)amino]ethyl}(methylsulfonyl)amino]-5-phenyl-1-benzofuran-3-carboxylate (60 mg, yield: 77%).

¹H-NMR (400 MHz, CDCl₃) δ 8.06˜8.10 (m, 3H), 7.59 (s, 1H), 7.49˜7.51 (m, 2H), 7.39˜7.46 (m, 3H), 7.14˜7.22 (m, 4H), 6.66˜6.70 (m, 1H), 6.54˜6.56 (m, 2H), 4.37˜4.42 (m, 2H), 3.23˜3.67 (m, 4H), 2.81 (s, 3H), 2.75 (s, 3H), 1.35 (t, J=7.2 Hz, 3H).

### Step 5: 2-(4-fluorophenyl)-6-[{2-[methyl(phenyl)amino]ethyl}(methylsulfonyl)amino-5-phenyl-1-benzofuran-3-carboxylic acid

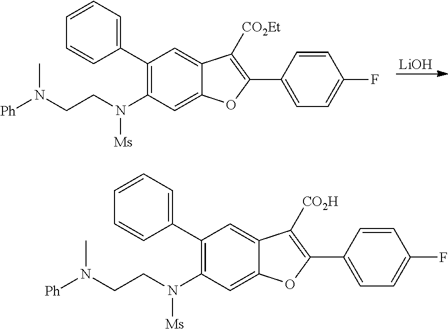

Step 5 was performed in an analogous manner to Example 133, Step 5. The crude product was purified by prep-TLC to give pure 2-(4-fluorophenyl)-6-[{2-[methyl(phenyl)amino]ethyl}(methylsulfonyl)amino-5-phenyl-1-benzofuran-3-carboxylic acid (50 mg, yield: 87%).

¹H-NMR (400 MHz, CDCl₃) δ 7.80˜7.89 (m, 3H), 7.50 (s, 1H), 7.07=7.42 (m, 10H), 6.97˜7.01 (m, 2H), 3.41˜3.67 (m, 4H), 2.94 (s, 3H), 2.71 (s, 3H).

### Step 6: 2-(4-fluorophenyl)-N-methyl-6-[{2-(methyl(phenyl)amino]ethyl}(methylsulfonyl)amino]-5-phenyl-1-benzofuran-3-carboxamide

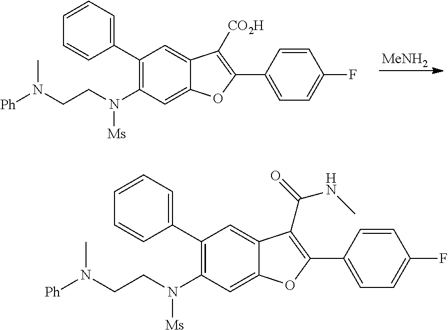

Step 6 was performed in an analogous manner to Example 133, Step 6. The crude product was purified by prep-TLC to give pure 2-(4-fluorophenyl)-N-methyl-6-[{2-[methyl(phenyl)amino]ethyl}(methylsulfonyl)amino]-5-phenyl-1-benzofuran-3-carboxamide (13 mg, yield: 42%).

¹H-NMR (400 MHz, CDCl₃) δ 7.90˜7.91 (m, 2H), 7.74 (s, 1H), 7.51 (s, 1H), 7.31˜7.43 (m, 5H), 7.08˜7.18 (m, 4H), 6.60˜6.63 (m, 1H), 6.48˜6.50 (m, 2H), 5.78 (s, 1H), 3.24˜3.41 (m, 4H), 2.92 (d, J=4.8 Hz, 3H), 2.74 (s, 3H), 2.70 (s, 3H).

### Example 135

### 5-(3-(benzo[d]thiazol-2-yl)phenyl)-2-(4-fluorophenyl)-N-methyl-6-(N-methylmethylsulfonamido)-1-benzofuran-3-carboxamide

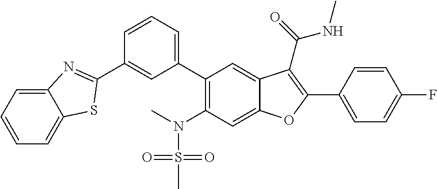

### Step 1: Methyl 2-(5-bromo-2-hydroxyphenyl)acetate

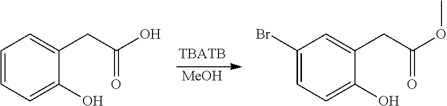

2-(2-hydroxyphenyl)acetic acid (100 g, 0.66 mol) was dissolved in MeOH, and then TBATB (320 g, 0.66 mmol) was added to the solution. The resulting mixture was stirred at RT for 18 hours. After evaporation of solvent, the residue was dissolved in diethyl ether. The organic layer was washed with 1 N HCl, 2 M sodium bisulfate, H₂O and brine, dried and evaporated to yield methyl 2-(5-bromo-2-hydroxyphenyl)acetate (145 g, yield: 90%).

¹H-NMR (400 MHz, CDCl₃) δ 7.48 (br s, 1H), 7.20˜7.25 (m, 2H), 6.75˜6.78 (m, 1H), 3.74 (s, 3H), 3.62 (s, 2H). MS (M+H)⁺: 245.

### Step 2: Methyl 2-(5-bromo-2-(tert-butyldimethylsilyloxy)phenyl)acetate

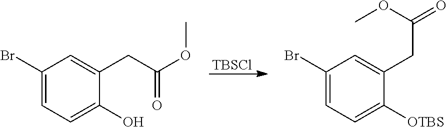

To a stirred solution of the product of Step 1 (1 g, 4.1 mmol) in DCM (5 mL) was added imidazole (0.56 g, 8.23 mmol) and TBSCl (0.93 g, 6.17 mmol) at 0° C. After stirred overnight at RT, the reaction mixture was washed with H₂O, brine and concentrated in vacuo, the residue was purified by column chromatography to furnish the pure product of methyl 2-(5-bromo-2-(tert-butyldimethylsilyloxy)phenyl)acetate (1.4 g, yield: 95%).

¹H-NMR (400 MHz, CDCl₃) δ 7.23 (d, J=2.4 Hz, 1H), 7.17 (dd, J₁=8.4 Hz, J₂=2.4 Hz, 1H), 6.61 (d, J=8.4 Hz, 1H), 3.61 (s, 3H), 3.50 (s, 2H), 0.91 (s, 9H), 0.15 (s, 6H). MS (M+H)⁺: 359.

### Step 3: Methyl 2-(5-bromo-2-(tert-butyldimethylsilyloxy)phenyl)-3-(4-fluorophenyl)-3-oxopropanoate

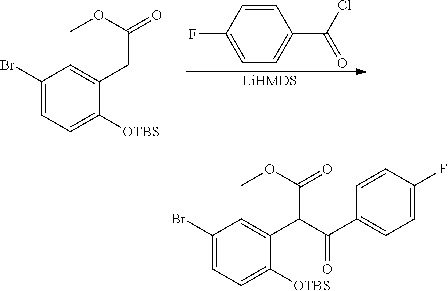

A solution of the product of Step 2 (500 mg, 1.4 mmol) in THF (10 mL) at −78° C. was treated dropwise with lithium bis(trimethylsilyl)amide (1.7 mL, 1.7 mmol, 1 N in THF). After stirred 30 minutes, a solution of 4-fluorobenzoyl chloride (250 mg, 1.6 mmol) in THF was added dropwise. The reaction mixture was stirred at −78° C. for 1 hour and at 0° C. for another 1 hour. The mixture was quenched with 1 N HCl, THF was removed in vacuo, and the residue was extracted with EtOAc. The organic layer was concentrated and purified by column chromatography to afford the pure product of methyl 2-(5-bromo-2-(tert-butyldimethylsilyloxy)phenyl)-3-(4-fluorophenyl)-3-oxopropanoate (550 mg, yield: 82%).

¹H-NMR (400 MHz, CDCl₃) δ 7.83˜7.87 (m, 2H), 7.28 (d, J=2.4 Hz, 1H), 7.16 (dd, J₁=8.4 Hz, J₂=2.4 Hz, 1H), 6.93˜6.98 (m, 2H), 6.63 (d, J=8.4 Hz, 1H), 5.86 (s, 1H), 3.65 (s, 3H), 0.91 (s, 9H), 0.18 (s, 3H), 0.10 (s, 3H). MS (M+H)⁺: 481.

### Step 4: Methyl 2-(5-bromo-2-hydroxyphenyl)-3-(4-fluorophenyl)-3-oxopropanoate

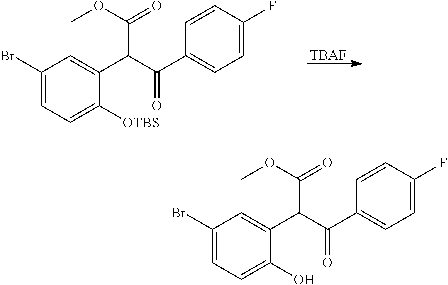

To a solution of the product of Step 3 (300 mg, 0.6 mmol) in THF (10 mL), TBAF (500 mg, 1.9 mmol) was added and the mixture was stirred at 0° C. for 1 hour. After concentrated in vacuo, the mixture was suspended in H₂O and extracted with EtOAc. The organic layer was washed with H₂O, brine and concentrated. The residue was purified by column chromatography to give the product of methyl 2-(5-bromo-2-hydroxyphenyl)-3-(4-fluorophenyl)-3-oxopropanoate (200 mg, yield: 87%).

¹H-NMR (400 MHz, CDCl₃) δ 7.99 (m, 2H), 7.33 (s, 1H), 7.18 (d, J=8.0 Hz, 1H), 7.07 (m, 2H), 6.68 (d, J=8.0 Hz, 1H), 5.93 (s, 1H), 3.77 (s, 3H). MS (M+H)⁺: 367.

### Step 5: Methyl 5-bromo-2-(4-fluorophenyl)-1-benzofuran-3-carboxylate

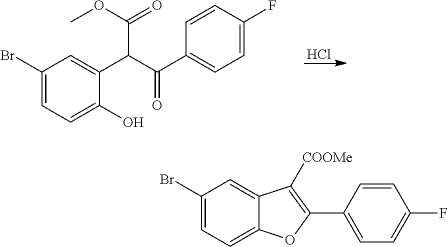

To a solution of the product of Step 4 (100 mg, 0.3 mmol) in acetone (4 mL) was added concentrated HCl, and the mixture was heated under reflux for 30 minutes. Then, the reaction mixture was concentrated in vacuo, suspended in H₂O and extracted with EtOAc. The organic layer was washed with H₂O, brine and concentrated. The residue was purified by prep-TLC to give pure methyl 5-bromo-2-(4-fluorophenyl)-1-benzofuran-3-carboxylate (70 mg, yield: 73%).

¹H-NMR (400 MHz, CDCl₃) δ 8.15 (s, 1H), 8.05 (m, 2H), 7.43 (m, 1H), 7.37 (m, 1H), 7.16 (m, 2H), 3.94 (s, 3H). MS (M+H)⁺: 349.

### Step 6: Methyl 5-bromo-2-(4-fluorophenyl)-6-nitro-1-benzofuran-3-carboxylate

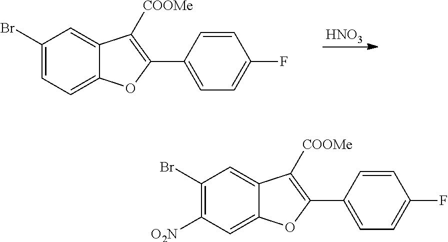

To a solution of the product of Step 5 (0.5 g, 1.4 mmol) in CHCl₃ (4 mL), fuming HNO₃ (1 mL) was added dropwise at RT, and the mixture was stirred for 4 hours. The reaction mixture was poured into ice water and extracted with EtOAc. The organic layer was washed with NaHCO₃ and brine. The solvent was removed by concentration to provide the crude product of methyl 5-bromo-2-(4-fluorophenyl)-6-nitro-1-benzofuran-3-carboxylate (0.4 g, yield: 70%). It was used for the next step without further purification.

### Step 7: Methyl 6-amino-5-bromo-2-(4-fluorophenyl)-1-benzofuran-3-carboxylate

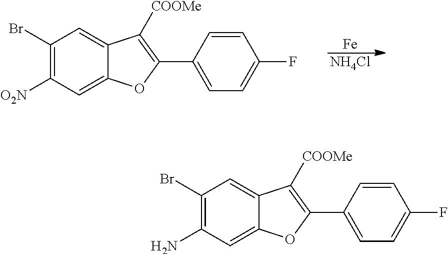

A mixture of the product of Step 6 (200 mg, 0.5 mmol), iron filings (200 mg, 3.58 mmol) and NH₄Cl (300 mg, 5.61 mmol) in MeOH:THF:H₂O (1:1:1, 20 mL) was stirred at reflux for 3 hours. After filtered and concentrated in vacuo, the residue was purified by column chromatography to furnish the pure methyl 6-amino-5-bromo-2-(4-fluorophenyl)-1-benzofuran-3-carboxylate (150 mg, yield: 81%).

¹H-NMR (400 MHz, CDCl₃) δ 7.99 (s, 1H), 7.96 (m, 2H), 7.05˜7.10 (m, 2H), 6.82 (s, 1H), 4.18 (br s, 2H), 3.86 (s, 3H). MS (M+H)⁺: 364.

### Step 8: Methyl 5-bromo-2-(4-fluorophenyl)-6-(methylsulfonamido)-1-benzofuran-3-carboxylate

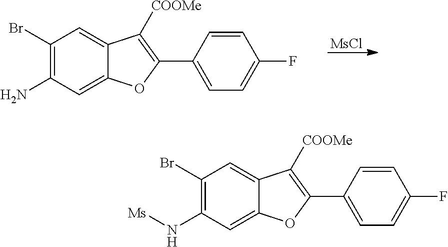

MsCl (60 μL, 0.77 mmol) was added to a solution of the product of Step 7 (150 mg, 0.41 mmol) and pyridine (0.34 mL) in dry DCM (10 mL) at 0° C. After stirring overnight at RT, the mixture was diluted with water, and extracted with DCM. The organic layer was washed with brine, dried over Na₂SO₄, filtered and concentrated in vacuo, and the residue was purified by prep-TLC to afford the pure product of methyl 5-bromo-2-(4-fluorophenyl)-6-(methylsulfonamido)-1-benzofuran-3-carboxylate (150 mg, yield: 82%).

¹H-NMR (400 MHz, CDCl₃) δ 8.21 (s, 1H), 7.99˜8.03 (m, 2H), 7.83 (s, 1H), 7.11˜7.16 (m, 2H), 6.82 (br s, 1H), 3.90 (s, 3H), 2.96 (s, 3H). MS (M+H)⁺: 442.

### Step 9: Methyl 5-bromo-2-(4-fluorophenyl)-6-(N-methylmethylsulfonamido)-1-benzofuran-3-carboxylate

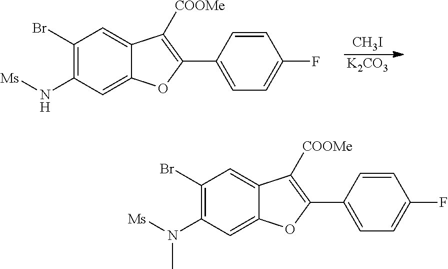

CH₃I (0.8 mL, 12.85 mmol) was added to a mixture of the product of Step 8 (5.0 g, 11.31 mmol), K₂CO₃ (3.2 g, 23.15 mmol) and KI (1.9 mg, 11.45 mmol) in DMF (40 mL) under N₂ protection. The mixture was stirred at reflux overnight. After filtered and concentrated in vacuo, the residue was purified by column chromatography to give the product of methyl 5-bromo-2-(4-fluorophenyl)-6-(N-methylmethylsulfonamido)-1-benzofuran-3-carboxylate (5 g, yield: 96%).

¹H-NMR (400 MHz, CDCl₃) δ 8.32 (s, 1H), 8.05˜8.09 (m, 2H), 7.72 (s, 1H), 7.17˜7.22 (m, 2H), 3.96 (s, 3H), 3.35 (s, 3H), 3.10 (s, 3H). MS (M+H)⁺: 456.

### Step 10: 5-bromo-2-(4-fluorophenyl)-6-(N-methylmethylsulfonamido)-1-benzofuran-3-carboxylic acid

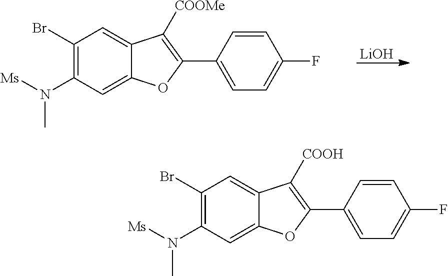

To a Solution of the product of Step 9 (5 g, 0.11 mol) in dioxane/H₂O (1:1, 100 mL) was added LiOH.H₂O (4.6 g, 0.11 mol), and the mixture was stirred at 100° C. for 2 hours. After concentration, the residue was dissolved in H₂O, 1 N HCl was added until pH reached 3, and the mixture was extracted with EtOAc. The organic layer was washed with brine, dried over Na₂SO₄ and filtered. The solvent was removed by distillation to provide the crude product of 5-bromo-2-(4-fluorophenyl)-6-(N-methylmethylsulfonamido)-1-benzofuran-3-carboxylic acid (4.5 g, yield: 97%). It was used for the next step without further purification.

### Step 11: 5-bromo-2-(4-fluorophenyl)-N-methyl-6-(N-methylmethylsulfonamido)-1-benzofuran-3-carboxamide

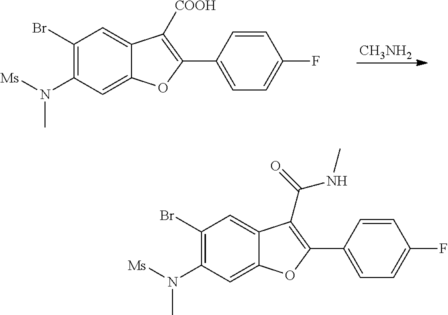

A solution of the product of Step 10 (5 g, 11.31 mmol), HOBT (3.2 g, 23.7 mmol) and EDCI (5.0 g, 26.1 mmol) in dry DMF (100 mL) was stirred at RT. After 30 minutes, Et₃N (16 mL) and CH₃NH₂ (HCl salt, 3.7 g, 56.5 mmol) was added to the mixture, and the mixture was stirred overnight. After the solvent was removed, H₂O was added, and the mixture was extracted with EtOAc. The combined organic layer was washed with H₂O and brine and concentrated. The residue was purified by column chromatography to give the product of 5-bromo-2-(4-fluorophenyl)-N-methyl-6-(N-methyl methylsulfonamido)-1-benzofuran-3-carboxamide (4.8 g, yield: 93%).

¹H-NMR (400 MHz, CDCl₃) δ 8.16 (s, 1H), 7.88˜7.92 (m, 2H), 7.70 (s, 1H), 7.18˜7.23 (m, 2H), 5.78 (br s, 1H), 3.34 (s, 3H), 3.09 (s, 3H), 3.00 (d, J=4.8 Hz, 3H). MS (M+H)⁺: 455.

### Step 12: 5-(3-(benzo[d]thiazol-2-yl)phenyl)-2-(4-fluorophenyl)-N-methyl-6-(N-methylmethylsulfonamido)-1-benzofuran-3-carboxamide

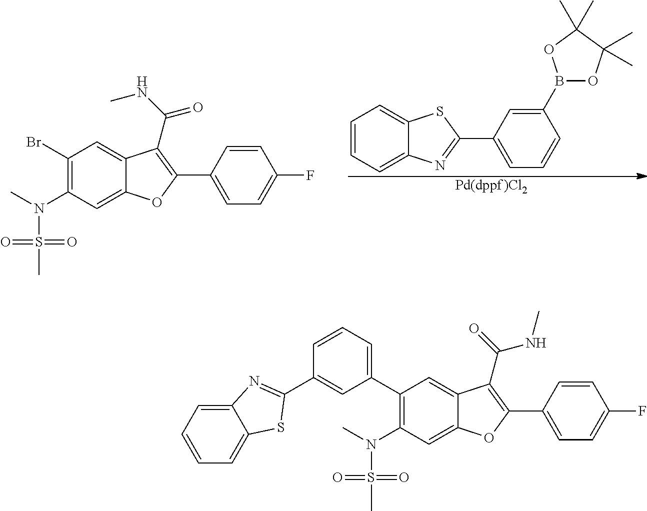

A mixture of Pd(dppf)Cl₂(10 mg), the product of Step 11 (50 mg, 0.11 mmol), K₃PO₄ (60 mg, 0.28 mmol) and 2-(3-(4,4,5,5-tetramethyl-1,3,2-dioxaborolan-2-yl)phenyl)benzo[d]thiazole (100 mg, 0.30 mmol) in DMF (2 mL) was stirred at 100° C. under N₂ protection overnight. Then, the solvent was removed, and H₂O was added. After extracted with EtOAc, the combined organic layer was dried over Na₂SO₄ and evaporated. The residue was purified by prep-HPLC to give the product of 5-(3-(benzo[d]thiazol-2-yl)phenyl)-2-(4-fluorophenyl)-N-methyl-6-(N-methylmethylsulfonamido)-1-benzofuran-3-carboxamide (20 mg, yield: 31%).

¹H-NMR (400 MHz, CDCl₃) δ 8.19 (s, 1H), 8.12 (d, J=7.2 Hz, 1H), 8.06 (d, J=8.4 Hz, 1H), 7.91˜7.96 (m, 3H), 7.86 (s, 1H), 7.58˜7.64 (m, 3H), 7.48˜7.53 (m, 1H), 7.38˜7.42 (m, 1H), 7.17˜7.22 (m, 2H), 6.03 (br s, 1H), 3.17 (s, 3H), 2.99 (d, J=4.8 Hz, 3H), 2.71 (s, 3H).

MS (M+H)⁺: 586.

### Examples 136-142

Examples 136-142 were prepared according to the general procedures of Example 135.

| Ex- | | | ¹H-NMR | MS |
|---|---|---|---|---|
| ample | Structure | Name | (400 MHz) | (M + H)⁺ |
| 136 | 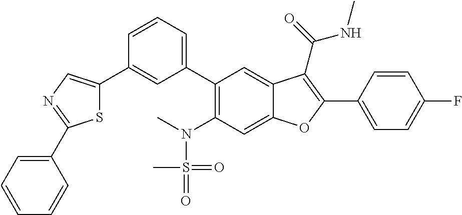 | 2-(4-fluorophenyl)- N-methyl-6-[methyl (methylsulfonyl) amino]-5-[3-(2- phenyl-1,3-thiazol- 5-yl)phenyl]-1- benzofuran-3- carboxamide | (CDCl₃) δ 8.27 (s, 1H), 7.83~7.93 (m, 4H), 7.74 (s, 1H), 7.65 (s, 1H), 7.60~7.63 (m, 2H), 7.47~7.54 (m, 5H), 7.22 (t, J = 6.4 Hz, 2H), 6.00 (d, J = 4.4 Hz, 1H), 3.13 (s, 3H), 2.99 (d, J = 5.2 Hz, 3H), 2.75 (s, 3H). | 612 |
| 137 | 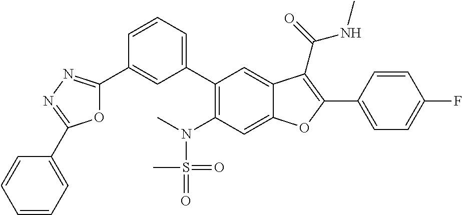 | 2-(4-fluorophenyl)- N-methyl-6-[methyl (methylsulfonyl) amino]-5-[3- (5-phenyl-1,3,4- oxadiazol-2- yl)phenyl]-1- benzofuran-3- carboxamide | (CDCl₃) δ 8.17 (s, 1H), 8.08~8.12 (m, 3H), 7.86~7.90 (m, 2H), 7.82 (s, 1H), 7.55~7.64 (m, 3H), 7.45~7.49 (m, 3H), 7.12~7.17 (m, 2H), 5.84 (s, 1H), 3.11 (s, 3H), 2.93~2.94 (d, J = 4.4 Hz, 3H), 2.67 (s, 3H). | 597 |
| 138 | 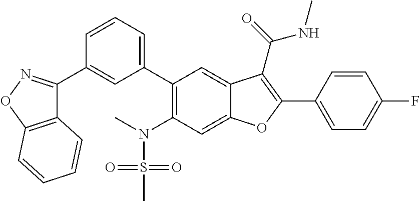 | 5-[3-(1,2- benzisoxazol-3- yl)phenyl]-2-(4- fluorophenyl)-N- methyl-6-[methyl (methylsulfonyl) amino]-1- benzofuran-3- carboxamide | (CDCl₃) δ 8.06 (s, 1H), 7.98~8.02 (m, 2H), 7.95~7.97 (m, 2H), 7.88 (s, 1H), 7.60~7.69 (m, 5H), 7.40 (t, J = 8.0 Hz, 1H), 7.22 (t, J = 8.4 Hz, 2H), 5.88 (s, 1H), 3.21 (s, 3H), 3.00 (d, J = 4.8 Hz, 3H), 2.68 (s, 3H). | 570 |
| 139 | 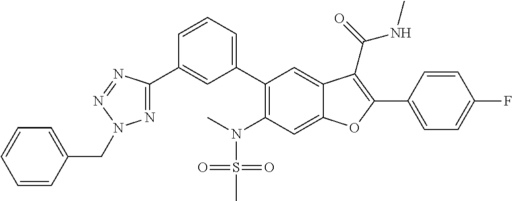 | 5-[3-(2-benzyl-2H- tetrazol-5-yl) phenyl]-2-(4- fluorophenyl)-N- methyl-6-[methyl (methylsulfonyl) amino]-1- benzofuran-3- carboxamide | (CDCl₃) δ 8.08~8.14 (m, 2H), 7.87~7.91 (m, 2H), 7.77 (s, 1H), 7.58 (s, 1H), 7.49~7.51 (m, 2H), 7.30~7.38 (m, 5H), 7.14 (t, J = 8.8 Hz, 2H), 5.79 (d, J = 4.4 Hz, 1H), 5.23 (s, 2H), 3.09 (s, 3H), 2.92 (d, J = 4.8 Hz, 3H), 2.54 (s, 3H). | 611 |
| 140 | 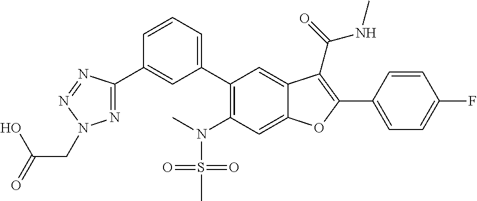 | [5-(3-{2-(4- fluorophenyl)-3- (methylcarbamoyl)- 6-[methyl (methylsulfonyl) amino]-1- benzofuran-5- yl}phenyl)-2H- tetrazol-2-yl]acetic acid | (MeOD) δ 8.25 (s, 1H), 8.17 (d, J = 6.4 Hz, 1H), 7.98~8.02 (m, 2H), 7.85 (s, 1H), 7.61~7.72 (m, 3H),7.29 (t, J = 8.4 Hz, 2H), 5.62 (s, 2H), 3.21 (s, 3H), 2.95 (s, 3H), 2.82 (s, 3H). | 579 |
| 141 | 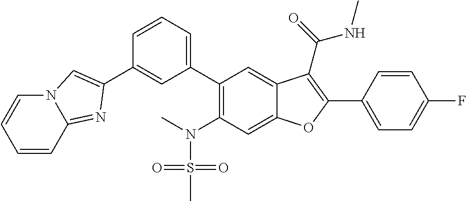 | 2-(4-fluorophenyl)- 5-[3-(imidazo[1,2- a]pyridin-2- yl)phenyl]-N- methyl-6-[methyl (methylsulfonyl) amino]-1- benzofuran-3- carboxamide | (CDCl₃) δ 8.35 (d, J = 5.2 Hz, 1H), 8.06 (s, 1H), 8.00 (d, J = 8.4 Hz, 2H), 7.86~7.89 (m, 2H), 7.66~7.73 (m, 3H), 7.50 (d, J = 7.2 Hz, 1H), 7.46 (s, 1H), 7.41 (t, J = 3.6 Hz, 1H), 7.24 (t, J = 6.0 Hz, 1H), 7.11 (t, J = 8.4 Hz, 2H), 6.98 (d, J = 3.6 Hz, 1H), 3.02 (s, 3H), 2.93 (d, J = 4.0 Hz, 3H), 2.86 (s, 3H). | 569 |
| 142 | 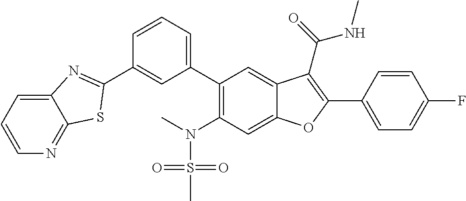 | 2-(4-fluorophenyl)- N-methyl-6-[methyl (methylsulfonyl) amino]-5-[3- ([1,3]thiazolo[5,4- b]pyridin-2- yl)phenyl]-1- benzofuran-3- carboxamide | (CDCl₃) δ 8.52 (s, 1H), 8.28~8.26 (d, J = 6.8 Hz, 1H), 8.26 (s, 1H), 8.18~8.12 (d, J = 4.8 Hz, 1H), 8.11~8.09 (m, 2H), 7.95 (s, 1H), 7.94~7.57 (m, 3H), 7.46~7.43 (m, 1H), 7.22~7.17 (t, J = 8.4 Hz, 2H), 5.89~5.88 (d, J = 4.0 Hz, 1H), 3.17 (s, 3H), 2.98~2.97 (d, J = 4.8 Hz, 3H), 2.69 (s, 3H). | 587 |

### Example 143

### 2-(4-fluorophenyl)-N-methyl-5-[3-(6-methyl-1,3-benzothiazol-2-yl)phenyl]-6-[methyl(methylsulfonyl)amino]-1-benzofuran-3-carboxamide

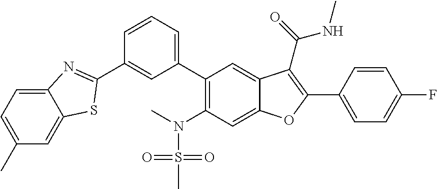

### Steps 1-11

Steps 1-11 were performed in an analogous manner to Example 135, Steps 1-11.

### Step 12: 2-(4-fluorophenyl)-5-(3-formylphenyl)-N-methyl-6-[methyl(methylsulfonyl)amino]-1-benzofuran-3-carboxamide

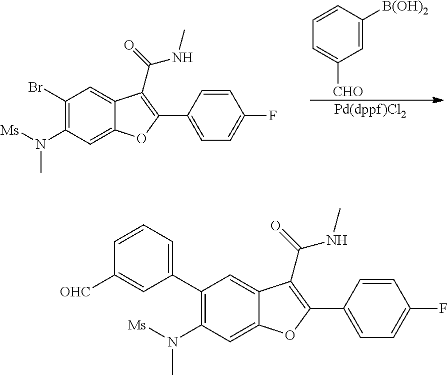

The aryl aldehyde (45 mg, yield: 73%) was prepared in an analogous manner to Example 136, Step 12.

### Step 13: 2-(4-fluorophenyl)-N-methyl-5-[3-(6-methyl-1,3-benzothiazol-2-yl)phenyl]-6-[methyl(methylsulfonyl)amino]-1-benzofuran-3-carboxamide

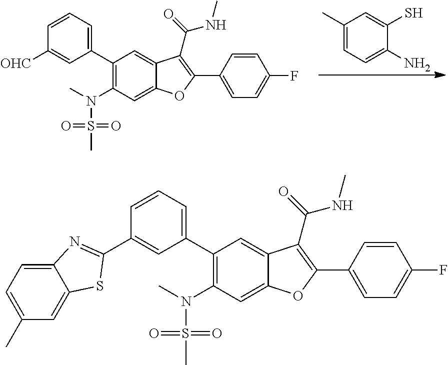

A mixture of 2-amino-5-methylbenzenethiol (50 mg, 0.10 mmol) and the aryl aldehyde (50 mg, 0.36 mmol) in DMSO was stirred at 200° C. for 1 hour. After cooling, 20 mL H₂O was added, and the mixture was extracted with EtOAc. The organic layer was washed with brine, dried over Na₂SO₄ and filtered. The solvent was removed, and the crude product was purified by prep-TLC to give pure 2-(4-fluorophenyl)-N-methyl-5-[3-(6-methyl-1,3-benzothiazol-2-yl)phenyl]-6-[methyl(methylsulfonyl)amino]-1-benzofuran-3-carboxamide (50 mg, yield: 82%).

¹H-NMR (400 MHz, CDCl₃) δ 8.19 (s, 1H), 8.12 (d, J=6.8 Hz, 1H), 7.94˜7.99 (m, 3H), 7.87 (s, 1H), 7.73 (s, 1H), 7.66 (s, 1H), 7.57˜7.61 (m, 2H), 7.33 (d, J=8.4 Hz, 1H), 7.19˜7.24 (m, 2H), 6.05 (br s, 1H), 3.19 (s, 3H), 3.01 (d, J=4.8 Hz, 3H), 2.72 (s, 3H), 2.53 (s, 3H). MS (M+H)⁺: 600.

### Examples 144-149

Examples 144-149 were prepared according to the general procedures of Example 143.

| Ex- | | | ¹H-NMR (400 MHz), | MS |
|---|---|---|---|---|
| ample | Structure | Name | (CDCl₃) δ | (M + H)⁺ |
| 144 | 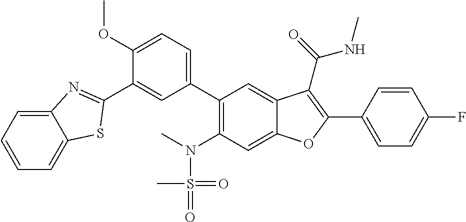 | 5-[3-(1,3- benzothiazol-2- yl)-4- methoxyphenyl]- 2-(4- fluorophenyl)- N-methyl- 6-[methyl (methylsulfonyl) amino]-1- benzofuran-3- carboxamide | 8.56 (s, 1H), 7.97~7.99 (d, J = 8.0 Hz, 1H), 7.87~7.92 (m, 3H), 7.79 (s, 1H), 7.59~7.61 (d, J = 8.0 Hz, 1H), 7.56 (s, 1H), 7.41~7.44 (m, 1H), 7.31~7.34 (m, 1H), 7.10~7.15 (m, 3H), 6.01 (s, 1H), 4.07 (s, 3H), 3.08~3.09 (d, J = 1.6 Hz, 3H), 2.93 (s, 3H), 2.75 (s, 3H). | 616 |
| 145 | 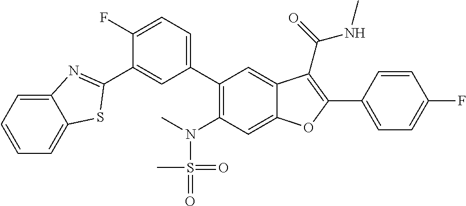 | 5-[3-(1,3- benzothiazol- 2-yl)-4- fluorophenyl]-2- (4-fluorophenyl)- N-methyl- 6-[methyl (methylsulfonyl) amino]-1- benzofuran-3- carboxamide | 8.41~8.43 (m, 1H), 8.03 (d, J = 8.0 Hz, 1H), 7.91~7.94 (m, 3H), 7.79 (s, 1H), 7.57~7.61 (m, 2H), 7.45~7.49 (m, 1H), 7.37~7.41 (m, 1H), 7.28~7.32 (m, 1H), 7.15 (t, J = 8.8 Hz, 2H), 3.12 (s, 3H), 2.94 (s, 3H), 2.79 (s, 3H). | 604 |
| 146 | 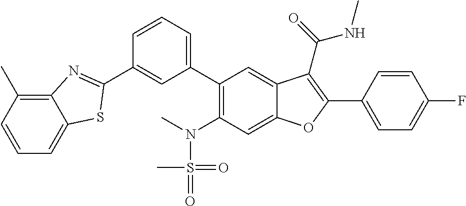 | 2- (4-fluorophenyl)- N-methyl-5-[3- (4-methyl-1,3- benzothiazol-2-yl) phenyl]-6-[methyl (methylsulfonyl) amino]-1- benzofuran-3- carboxamide | 8.16 (s, 1H), 8.04~8.06 (m, 1H), 7.87~7.91 (m, 2H), 7.82 (s, 1H), 7.67 (t, J = 4.4 Hz, 1H), 7.60 (s, 1H), 7.51~7.53 (m, 2H), 7.22 (d, J = 5.2 Hz, 2H), 7.12~7.16 (m, 2H), 5.82 (d, J = 4.4 Hz, 1H), 3.12 (s, 3H), 2.93 (d, J = 4.8 Hz, 3H), 2.72 (s, 3H), 2.63 (s, 3H). | 600 |
| 147 | 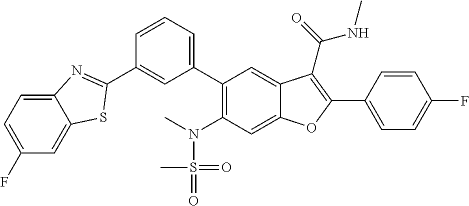 | 5-[3-(6-fluoro- 1,3-benzothiazol- 2-yl)phenyl]-2-(4- fluorophenyl)-N- methyl-6-[methyl (methylsulfonyl) amino]-1- benzofuran-3- carboxamide | 8.20 (s, 1H), 8.12 (d, J = 6.8 Hz, 1H), 8.02~8.05 (m, 1H), 7.97~8.00 (m, 2H), 7.91 (s, 1H), 7.68 (s, 1H), 7.66~7.60 (m, 3H), 7.24 (t, J = 8.8 Hz, 3H), 5.94 (d, J = 4.0 Hz, 1H), 3.20 (s, 3H), 3.03~3.02 (m, 3H), 2.75 (s, 3H). | 604 |
| 148 | 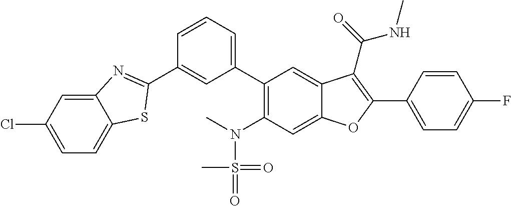 | 5-[3-(5-chloro- 1,3-benzothiazol- 2-yl)phenyl]-2-(4- fluorophenyl)-N- methyl-6-[methyl (methylsulfonyl) amino]-1- benzofuran-3- carboxamide | 8.20 (s, 1H), 8.11~8.08 (m, 1H), 8.04~8.02 (d, J = 2.0 Hz, 1H), 7.96~7.92 (m, 2H), 7.86 (s, 1H), 7.82~7.79 (d, J = 8.4 Hz, 1H), 7.63~7.56 (m, 3H), 7.37~7.34 (m, 1H), 7.22~7.17 (m, 2H), 5.88~5.86 (m, 1H), 3.15 (s, 3H), 2.98 (d, J = 5.2 Hz, 3H), 2.71 (s, 3H). | 620 |
| 149 | 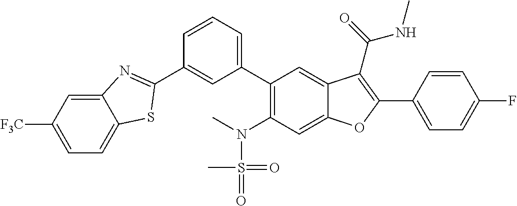 | 2- (4-fluorophenyl)- N-methyl- 6-[methyl (methylsulfonyl) amino]-5-{3-[5- (trifluoromethyl)- 1,3-benzothiazol- 2-yl]phenyl}-1- benzofuran-3- carboxamide | 8.31 (s, 1H), 8.21 (s, 1H), 8.20~8.11 (m, 1H), 8.02~8.00 (d, J = 8.4 Hz, 1H), 7.96~7.94 (m, 2H), 7.88 (s, 1H), 7.64~7.59 (m, 4H), 7.23~7.17 (m, 2H), 5.87~5.85 (m, 1H), 3.15 (s, 3H), 2.98 (d, J = 4.8 Hz, 3H), 2.73 (s, 3H). | 654 |

### Example 150

### 5-[3-(5-fluoro-1H-benzimidazol-2-yl)phenyl]-2-(4-fluorophenyl)-N-methyl-6-[methyl(methylsulfonyl)amino]-1-benzofuran-3-carboxamide

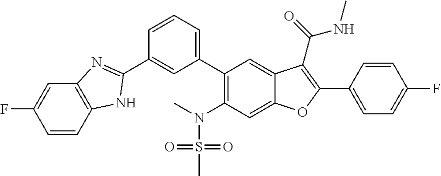

### Steps 1-12

Steps 1-12 were performed in an analogous manner to Example 143, Steps 1-12.

### Step 13: 5-[3-(5-fluoro-1H-benzimidazol-2-yl)phenyl]-2-(4-fluorophenyl)-N-methyl-6-[methyl(methylsulfonyl)amino]-1-benzofuran-3-carboxamide

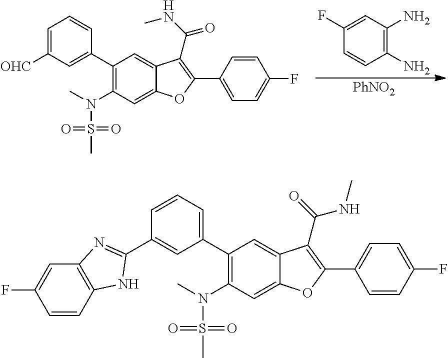

The aryl aldehyde of Example 143, Step 12 (100 mg, 0.21 mmol) and 4-fluorobenzene-1,2-diamine (32 mg, 0.25 mmol) were added in PhNO₂ (4 mL) and the mixture was heated to 120° C. and stirred overnight. The mixture was concentrated, and H₂O (30 mL) was added. After extraction with EtOAc, the organic layer was washed with brine and concentrated. The residue was purified by prep-HPLC to give pure 5-[3-(5-fluoro-1H-benzimidazol-2-yl)phenyl]-2-(4-fluorophenyl)-N-methyl-6-[methyl(methyl sulfonyl)amino]-1-benzofuran-3-carboxamide (30 mg, yield: 41.5%).

¹H-NMR: (400 MHz, CDCl₃) δ 8.16 (s, 1H), 8.00 (d, J=6.8 Hz, 1H), 7.87 (m, 2H), 7.72 (s, 1H), 7.56˜7.58 (m, 1H), 7.48˜7.50 (m, 1H), 7.41 (s, 1H), 7.28˜7.35 (m, 2H), 7.04˜7.14 (m, 3H), 6.62˜6.68 (m, 1H), 2.93˜2.96 (m, 9H). MS (M+H)⁺: 587.

### Examples 151-154

Examples 151-154 were prepared according to the general procedures of Example 150.

| Ex- | | | | MS |
|---|---|---|---|---|
| ample | Structure | Name | ¹H-NMR (400 MHz) | (M + H)⁺ |
| 151 | 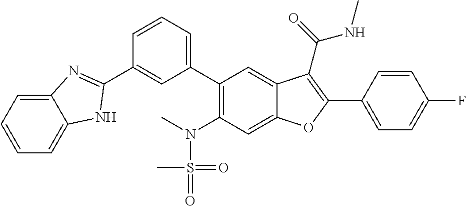 | 5-[3-(1H- benzimidazol-2- yl)phenyl]-2-(4- fluorophenyl)-N- methyl-6-[methyl (methylsulfonyl) amino]-1- benzofuran-3- carboxamide | (MeOD) δ 8.13 (s, 1H), 8.05 (d, J = 8.4 Hz, 1H), 7.86 (t, J = 7.6 Hz, 3H), 7.82 (d, J = 7.6 Hz, 1H), 7.71~7.74 (m, 4H), 7.53 (t, J = 3.2 Hz, 2H), 7.20 (t, J = 8.4 Hz, 2H), 3.15 (s, 3H), 2.86 (s, 3H), 2.84 (s, 3H). | 569 |
| 152 | 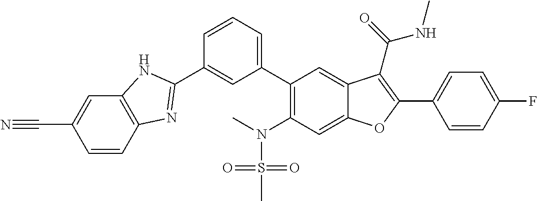 | 5-[3-(6-cyano-1H- benzimidazol-2- yl)phenyl]-2-(4- fluorophenyl)-N- methyl-6-[methyl (methylsulfonyl) amino]-1- benzofuran-3- carboxamide | (CDCl₃) δ 8.18 (s, 1H), 8.11 (m, 1H), 7.92 (s, 1H), 7.81~7.86 (m, 3H), 7.67 (d, J = 8.4 Hz, 1H), 7.47~7.54 (m, 3H), 7.42 (s, 1H), 7.12~7.19 (m, 2H), 6.18 (br s, 1H), 3.06 (s, 3H), 2.95 (s, 3H), 2.87 (s, 3H). | 594 |
| 153 | 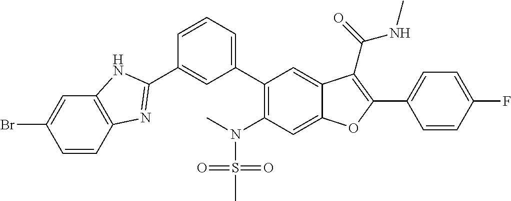 | 5-[3-(6-bromo-1H- benzimidazol-2- yl)phenyl]-2-(4- fluorophenyl)-N- methyl-6-[methyl (methylsulfonyl) amino]-1- benzofuran-3- carboxamide | (CDCl₃) δ 8.14 (s, 1H), 8.06 (d, J = 6.4 Hz, 1H), 7.91 (s, 2H), 7.72~7.63 (m, 2H), 7.33~7.47 (m, 5H), 7.18 (t, J = 8.0 Hz, 2H), 6.60 (s, 1H), 3.00 (s, 3H), 2.99 (s, 3H), 2.93 (s, 3H). | 647 |
| 154 | 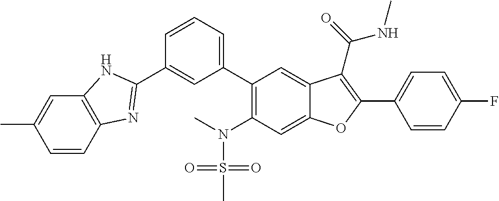 | 2-(4-fluorophenyl)- N-methyl-5-[3-(6- methyl-1H- benzimidazol-2- yl)phenyl]-6-[methyl (methylsulfonyl) amino]-1- benzofuran-3- carboxamide | (CDCl₃) δ 8.09 (s, 1H), 7.95~7.97 (d, J = 7.6 Hz, 1H), 7.77~7.81 (m, 2H), 7.68 (s, 1H), 7.63~7.65 (d, J = 8.0 Hz, 1H), 7.51 (s, 1H), 7.48~7.50 (d, J = 8.4 Hz, 2H), 7.39 (s, 1H), 7.19~7.21 (d, J = 7.2 Hz, 1H), 7.04~7.09 (m, 2H), 3.04 (s, 3H), 2.81 (s, 3H), 2.75 (s, 3H), 2.40 (s, 3H). | 583 |

### Example 155

### 2-(4-fluorophenyl)-N-methyl-6-(N-methylmethylsulfonamido)-5-(3-(oxazolo[4,5-b]pyridin-2-yl)phenyl)benzofuran-3-carboxamide

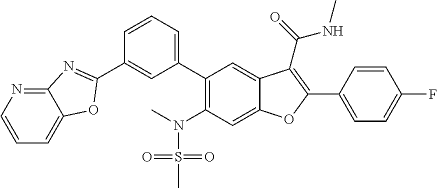

### Step 1: Methyl 2-(5-bromo-2-hydroxyphenyl)acetate

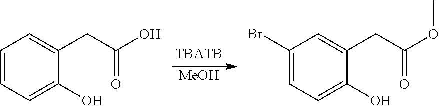

2-(2-hydroxyphenyl)acetic acid (484 g, 3.18 mol) was dissolved in methanol, and then tetrabutylammonium tribromide (1549 g, 3.18 mol) was added to the solution. The resulting mixture was allowed to stir at room temperature for 18 hours. After evaporation of solvent in vacuo, the residue obtained was dissolved in EtOAc. The organic layer was washed with 1 N HCl, water and brine, dried and concentrated, the residue obtained was purified using flash column chromatography on silica gel (eluted with PE/EtOAc=10/1) to give pure methyl 2-(5-bromo-2-hydroxyphenyl)acetate (750 g, 94%). ¹H-NMR (400 MHz, CDCl₃) δ 7.48 (br s, 1H), 7.20˜7.25 (m, 2H), 6.75˜6.78 (m, 1H), 3.74 (s, 3H), 3.62 (s, 2H). MS (M+H)⁺: 245.

### Step 2: Methyl 2-(5-bromo-2-(tert-butyldimethylsilyloxy)phenyl)acetate

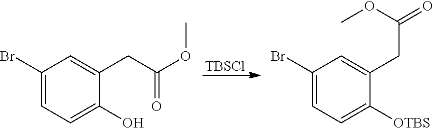

To a stirring solution of methyl 2-(5-bromo-2-hydroxyphenyl)acetate (750 g, 3.06 mol) in dichloromethane (4 L) was added imidazole (416 g, 6.1 mol) and TBSCl (692 g, 4.6 mol) at 0° C. After stirred for about 15 hours at room temperature, the reaction mixture was washed with water, brine and concentrated in vacuo, the residue obtained was purified using flash column chromatography on silica gel (eluted with PE/EtOAc=30/1) to furnish pure product of methyl 2-(5-bromo-2-(tertbutyldimethylsilyloxy)phenyl)acetate (880 g, 80%). ¹H-NMR (400 MHz, CDCl₃) δ 7.23 (d, J=2.4 Hz, 1H), 7.17 (dd, J₁=8.4 Hz, J₂=2.4 Hz, 1H), 6.61 (d, J=8.4 Hz, 1H), 3.61 (s, 3H), 3.50 (s, 2H), 0.91 (s, 9H), 0.15 (s, 6H). MS (M+H)⁺: 359.

### Step 3: Methyl 2-(5-bromo-2-(tert-butyldimethylsilyloxy)phenyl)-3-(4-fluorophenyl)-3

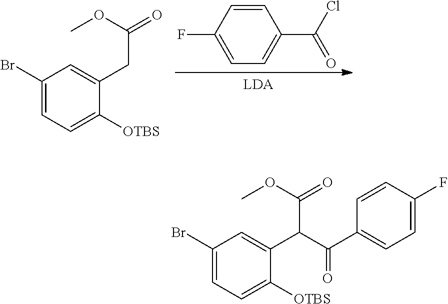

A solution of methyl 2-(5-bromo-2-(tert-butyldimethylsilyloxy)phenyl)acetate (220 g, 0.62 mol) in THF (1.5 L) at −78° C. was treated dropwise with a THF solution of LDA (0.74 mol, freshly prepared from i-Pr₂NH and n-BuLi). After stirred for 1 hour, a solution of 4-fluorobenzoyl chloride (106 g, 0.68 mol) in THF was added dropwise. The reaction mixture was allowed to stir at −78° C. for 1 hour and at 0° C. for another 1 hours. The mixture was quenched with 1 N HCl, and then THF was removed in vacuo, the residue obtained was extracted with EtOAc. The organic layer was concentrated and purified using flash column chromatography on silica gel (eluted with PE/EtOAc=10/1) to provide pure product of methyl 2-(5-bromo-2-(tert-butyldimethylsilyloxy)phenyl)-3-(4-fluorophenyl)-3-oxopropanoate (236 g, 80%). ¹H-NMR (400 MHz, CDCl₃) δ 7.83˜7.87 (m, 2H), 7.28 (d, J=2.4 Hz, 1H), 7.16 (dd, J₁=8.4 Hz, J₂=2.4 Hz, 1H), 6.93˜6.98 (m, 2H), 6.63 (d, J=8.4 Hz, 1H), 5.86 (s, 1H), 3.65 (s, 3H), 0.91 (s, 9H), 0.18 (s, 3H), 0.10 (s, 3H). MS (M+H)⁺: 481.

### Step 4: Methyl 2-(5-bromo-2-hydroxyphenyl)-3-(4-fluorophenyl)-3-oxopropanoate

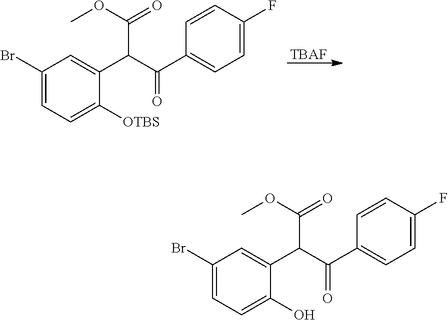

TBAF (217.5 g, 0.83 mol) was added to a solution of methyl 2-(5-bromo-2-(tert-butyldimethylsilyloxy)phenyl)-3-(4-fluorophenyl)-3-oxopropanoate (267 g, 554.6 mol) in THF (2 L), and the mixture was allowed to stir at 0° C. for 1 hours. The reaction mixture was then concentrated in vacuo and the resulting residue was suspended in H₂O and extracted with ethyl acetate. The organic layer was washed with H₂O, brine and concentrated in vacuo. The residue obtained was purified using flash column chromatography on silica gel (eluted with PE/EtOAc from 10/1 to 5/1) to provide methyl 2-(5-bromo-2-hydroxyphenyl)-3-(4-fluorophenyl)-3-oxopropanoate (178.6 g, 88%). ¹H-NMR (400 MHz, CDCl₃) δ 7.99 (m, 2H), 7.33 (s, 1H), 7.18 (d, J=8.0 Hz, 1H), 7.07 (m, 2H), 6.68 (d, J=8.0 Hz, 1H), 5.93 (s, 1H), 3.77 (s, 3H). MS (M+H)⁺: 367.

### Step 5: Methyl 5-bromo-2-(4-fluorophenyl)-1-benzofuran-3-carboxylate

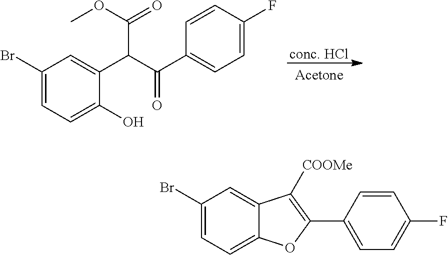

To a solution of methyl 2-(5-bromo-2-hydroxyphenyl)-3-(4-fluorophenyl)-3-oxopropanoate (50 g, 136.1 mmol) in acetone (200 mL) was added concentrated hydrochloric acid and the mixture was heated to reflux for 1 hour. Then the reaction mixture was concentrated in vacuo, suspended in H₂O and extracted with ethyl acetate. The organic layer was washed with aq. NaHCO₃ and brine. Then the organic layer was concentrated to provide the crude product of methyl 5-bromo-2-(4-fluorophenyl)-1-benzofuran-3-carboxylate. It was used for the next step without further purification. ¹H-NMR (400 MHz, CDCl₃) δ 8.15 (s, 1H), 8.05 (m, 2H), 7.43 (m, 1H), 7.37 (m, 1H), 7.16 (m, 2H), 3.94 (s, 3H). MS (M+H)⁺: 349.

### Step 6: Methyl 5-bromo-2-(4-fluorophenyl)-6-nitro-1-benzofuran-3-carboxylate

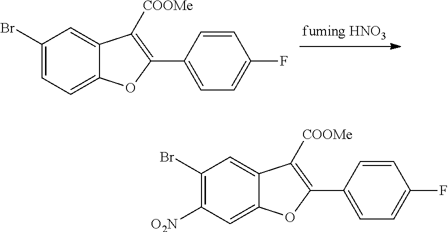

To a solution of methyl 5-bromo-2-(4-fluorophenyl)-1-benzofuran-3-carboxylate (50 g, 143.2 mmol) in CHCl₃ (300 mL) at room temperature, was added dropwise fuming HNO₃ (50 mL) and the reaction was allowed to stir for 4 hours. The reaction mixture was poured into ice water and extracted with ethyl acetate. The organic layer was washed with NaHCO₃ and brine, then concentrated in vacuo to provide methyl 5-bromo-2-(4-fluorophenyl)-6-nitro-1-benzofuran-3-carboxylate, which was used without further purification.

### Step 7: Methyl 6-amino-5-bromo-2-(4-fluorophenyl)-1-benzofuran-3-carboxylate

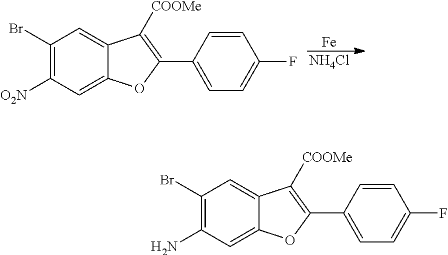

A mixture of methyl 5-bromo-2-(4-fluorophenyl)-6-nitro-1-benzofuran-3-carboxylate (100 g, crude), iron filings (100 g, 1.79 mol) and NH₄Cl (200 g, 3.74 mol) in MeOH/THF/H₂O (8/8/5, 1 L) was heated to reflux and allowed to stir at this temperature for 3 hours. The reaction mixture was then filtered and concentrated in vacuo, the residue obtained was purified using flash column chromatography on silica gel (eluted with PE/EtOAc=10/1 and then with pure dichloromethane) to furnish pure product of methyl 6-amino-5-bromo-2-(4-fluorophenyl)-1-benzofuran-3-carboxylate (41.2 g, 44.5%, 3 steps overall). ¹H-NMR (400 MHz, CDCl₃) δ 7.99 (s, 1H), 7.96 (m, 2H), 7.05˜7.10 (m, 2H), 6.82 (s, 1H), 4.18 (br s, 2H), 3.86 (s, 3H). MS (M+H)⁺: 364.

### Step 8: Methyl 5-bromo-2-(4-fluorophenyl)-6-(methylsulfonamido)-1-benzofuran-3-carboxylate

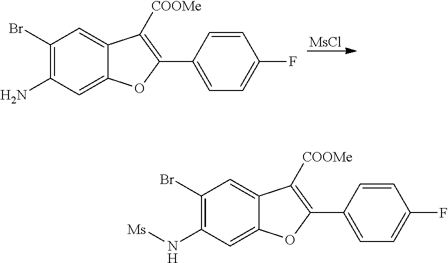

MSCl (25.2 g, 219.7 mmol) was added to a solution of methyl 6-amino-5-bromo-2-(4-fluorophenyl)-1-benzofuran-3-carboxylate (40 g, 109.8 mmol) and pyridine (26.1 g, 329.5 mmol) in dry dichloromethane (300 mL) at 0° C. After stirred for about 15 hours at room temperature, the mixture was diluted with water, and extracted with dichloromethane. The organic layer was washed with brine, dried over Na₂SO₄, filtered and concentrated in vacuo. The residue obtained was crystallized from EtOAc to provide the product of methyl 5-bromo-2-(4-fluorophenyl)-6-(methylsulfonamido)-1-benzofuran-3-carboxylate (38.2 g, 78.6%). ¹H-NMR (400 MHz, CDCl₃) δ 8.21 (s, 1H), 7.99˜8.03 (m, 2H), 7.83 (s, 1H), 7.11˜7.16 (m, 2H), 6.82 (br s, 1H), 3.90 (s, 3H), 2.96 (s, 3H). MS (M+H)⁺: 442.

### Step 9: Methyl 5-bromo-2-(4-fluorophenyl)-6-(N-methylmethylsulfonamido)-1-benzofuran-3-carboxylate

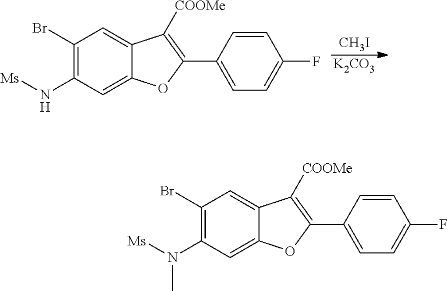

CH₃I (3.53 g, 24.9 mmol) was added to a mixture of methyl 5-bromo-2-(4-fluorophenyl)-6-(methylsulfonamido)-1-benzofuran-3-carboxylate (10 g, 22.61 mmol), K₂CO₃ (6.25 g, 45.2 mmol) and KI (1.88 g, 11.31 mmol) in DMF (100 mL) under N₂ protection. The mixture was allowed to stir at reflux for about 15 hours. After concentrated, H₂O was added and the mixture was extracted with dichloromethane. The combined organic layer was washed with H₂O, brine and concentrated in vacuo. The residue obtained was crystallized from EtOAc to provide methyl 5-bromo-2-(4-fluorophenyl)-6-(N-methylmethylsulfonamido)-1-benzofuran-3-carboxylate (9.6 g, 93%). ¹H-NMR (400 MHz, CDCl₃) δ 8.32 (s, 1H), 8.05˜8.09 (m, 2H), 7.72 (s, 1H), 7.17=7.22 (m, 2H), 3.96 (s, 3H), 3.35 (s, 3H), 3.10 (s, 3H). MS (M+H)⁺: 456.

### Step 10—5-bromo-2-(4-fluorophenyl)-6-(N-methylmethylsulfonamido)-1-benzofuran-3-carboxylic acid

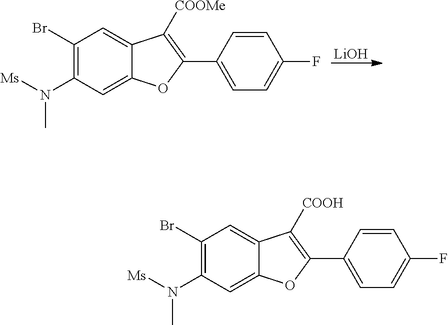

To a solution of methyl 5-bromo-2-(4-fluorophenyl)-6-(N-methylmethylsulfonamido)-1-benzofuran-3-carboxylate (20 g, 43.8 mmol) in dioxane/H₂O (1/1,100 mL) was added LiOH.H₂O (18.39 g, 0.44 mol), and the mixture was heated to reflux for 3 hours, filtered and concentrated in vacuo. The residue obtained was dissolved in H₂O, 1 N HCl was added until pH reached 3, and the mixture was extracted with dichloromethane. The organic layer was washed with brine, dried over Na₂SO₄ and filtered. The solvent was removed by concentration to provide the crude product of 5-bromo-2-(4-fluorophenyl)-6-(N-methylmethylsulfonamido)-1-benzofuran-3-carboxylic acid (18.2 g, 93.8%). It was used for the next step without further purification.

Step 11—5-bromo-2-(4-fluorophenyl)-N-methyl-6-(N-methylmethylsulfonamido)-1-benzofuran-3-carboxamide (Compound L)

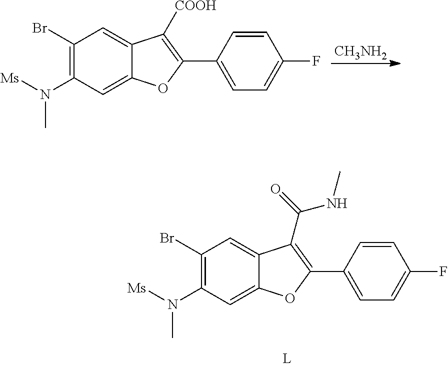

A solution of 5-bromo-2-(4-fluorophenyl)-6-(N-methylmethylsulfonamido)-1-benzofuran-3-carboxylic acid (21 g, 47.5 mmol), HOBT (7.06 g, 52.2 mmol) and EDCI (9 g, 47.5 mmol) in dry DMF (200 mL) was allowed to stir at room temperature. After 30 minutes, Et₃N (16 mL) and CH₃NH₂ (HCl salt, 6.41 g, 95 mmol) was added to the mixture, and the mixture was allowed to stir for about 15 hours. After the solvent was removed, H₂O was added and the mixture was extracted with dichloromethane. The combined organic layer was washed with H₂O, brine and concentrated in vacuo. The residue obtained was crystallized from EtOAc to provide compound L (19.5 g, 90%). ¹H-NMR (400 MHz, CDCl₃) δ 8.16 (s, 1H), 7.88˜7.92 (m, 2H), 7.70 (s, 1H), 7.18˜7.23 (m, 2H), 5.78 (br s, 1H), 3.34 (s, 3H), 3.09 (s, 3H), 3.00 (d, J=4.8 Hz, 3H). MS (M+H)⁺: 455.

### Step 12—2-(4-fluorophenyl)-N-methyl-6-(N-methylmethylsulfonamido)-5-(3-(oxazolo[4,5-b]pyridin-2-yl)phenyl)benzofuran-3-carboxamide

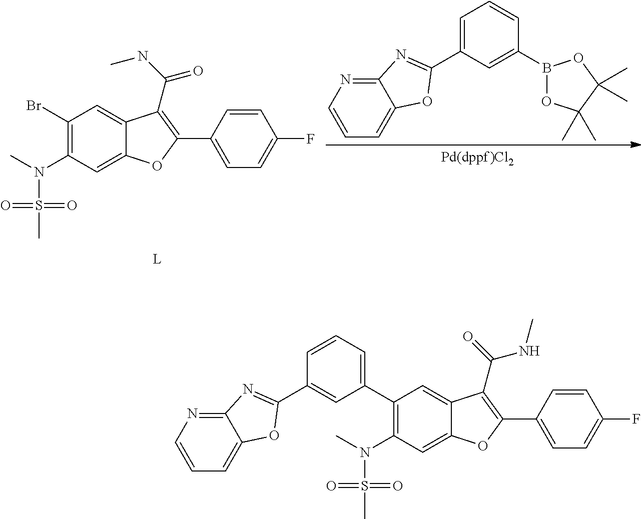

To a degassed solution of 2-[3-(4,4,5,5-Tetramethyl-[1,3,2]dioxaborolan-2-yl)-phenyl]-oxazolo[4,5-b]pyridine (prepared from corresponding bromide, 587 mg, 1.82 mmol) was added a solution of Compound L (635 mg, 1.40 mmol) and K₃PO₄ (771 mg, 3.64 mmol) in dry DMF (6 mL). To the resulting solution was added Pd(dppf)Cl₂ (30 mg) and the reaction mixture was placed under N₂ atmosphere, heated to 100° C. and allowed to stir at this temperature for 6 hours. After cooled to room temperature and filtered, the filtrate was washed with H₂O, brine, and dried over Na₂SO₄, filtered and concentrated in vacuo. The residue obtained was purified using column chromatography (PE:EtOAc=1:1) to provide the target compound (430 mg, 53.9%) as white solid. ¹H-NMR (CDCl₃, 400 MHz) δ 8.60˜8.61 (m, 1H), 8.39 (s, 1H), 8.33 (d, J=6.8 Hz, 1H), 7.91˜7.95 (m, 3H), 7.88 (s, 1H), 7.72 (d, J=7.6 Hz, 1H), 7.62˜7.66 (m, 2H), 7.35˜7.38 (m, 1H), 7.20 (d, J=8.8 Hz, 2H), 5.93˜5.94 (m, 1H), 3.18 (s, 3H), 2.99 (d, J=4.8 Hz, 3H), 2.71 (s, 3H). MS (M+H)⁺: 571.

The following compounds of the present invention were prepared using the method described in Example 155 and substituting the appropriate reactants and/or reagents.

| | | | MS |
|---|---|---|---|
| Example | Structure | NMR | (M + H)⁺ |
| 156 | 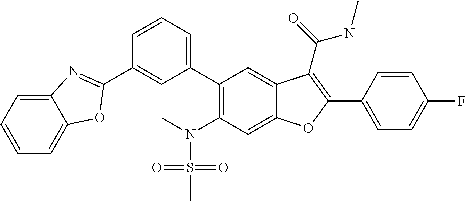 | ¹H-NMR (CDCl₃, 400 MHz) δ 8.30~8.36 (m, 2H), 7.98 (s, 2H), 7.90 (s, 1H), 7.80 (s, 1H), 7.62~7.68 (m, 4H), 7.40 (s, 2H), 7.21~7.25 (m, 2H), 5.97 (s, 1H), 3.21 (s, 3H), 3.03 (s, 3H), 2.71 (s, 3H). | 570 |
| 157 | 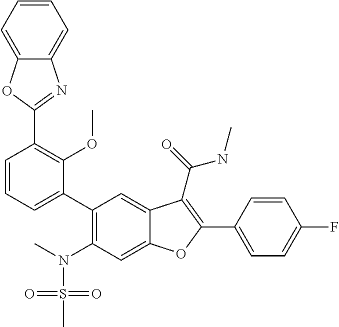 | ¹H-NMR (CDCl₃, 400 MHz) δ 8.10 (d, J = 8.0 Hz, 1H), 7.79~7.85 (m, 4H), 7.52~7.59 (m, 3H), 7.31~7.37 (m, 3H), 7.16 (t, J = 8.8 Hz, 2H), 5.91 (d, J = 4.8 Hz, 1H), 3.45 (s, 3H), 3.19 (s, 3H), 2.94 (d, J = 4.8 Hz, 3H), 2.42 (s, 3H). | 600 |
| 158 | 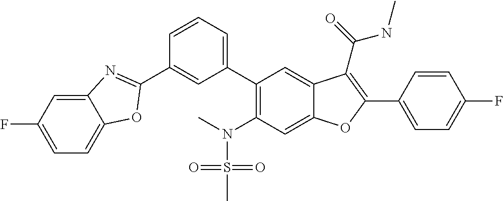 | ¹H-NMR (CDCl₃, 400 MHz) δ 7.92~7.95 (m, 2H), 7.87 (s, 1H), 7.59~7.68 (m, 3H), 7.49~7.52 (m, 2H), 7.42~7.45 (m, 2H), 7.20 (t, J = 8.8 Hz, 2H), 7.06~7.11 (m, 1H), 5.87 (d, J = 4.4 Hz, 1H), 3.17 (s, 3H), 2.97 (d, J = 4.8 Hz, 3H), 2.68 (s, 3H). | 588 |
| 159 | 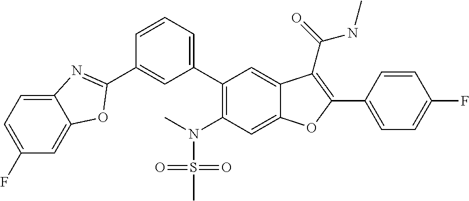 | ¹H-NMR (CDCl₃, 400 MHz) δ 8.33 (s, 1H), 8.27 (d, J = 5.2 Hz, 1H), 7.96~8.00 (m, 2H), 7.91 (s, 1H), 7.65~7.76 (m, 3H), 7.34~7.37 (m, 2H), 7.22~7.27 (m, 2H), 7.13~7.18 (m, 1H), 5.87 (s, 1H), 3.22 (s, 3H), 3.04 (d, J = 4.8 Hz, 3H), 2.72 (s, 3H). | 588 |
| 160 | 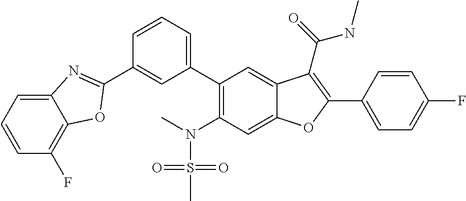 | ¹H-NMR (CDCl₃, 400 MHz) δ 8.37 (s, 1H), 8.33 (d, J = 7.6 Hz, 1H), 7.96~7.99 (m, 2H), 7.90 (s, 1H), 7.64~7.74 (m, 3H), 7.59 (d, J = 8.0 Hz, 1H), 7.31~7.36 (m, 1H), 7.23 (t, J = 8.4 Hz, 2H), 7.15 (t, J = 9.2 Hz, 1H), 6.03 (d, J = 4.4 Hz, 1H), 3.22 (s, 3H), 3.02 (d, J = 4.8 Hz, 3H), 2.73 (s, 3H). | 588 |
| 161 | 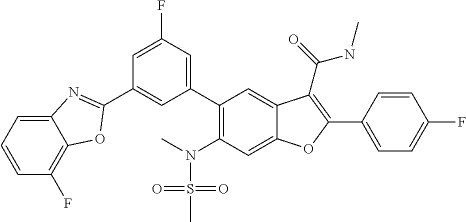 | ¹H-NMR (CDCl₃, 400 MHz) δ 8.07 (s, 1H), 7.82~7.93 (m, 4H), 7.58 (s, 1H), 7.50 (d, J = 8.0 Hz, 1H), 7.35 (d, J = 8.8 Hz, 1H), 7.23~7.28 (m, 1H), 7.14 (t, J = 8.8 Hz, 2H), 7.08 (t, J = 8.6 Hz, 1H), 5.87 (d, J = 4.4 Hz, 1H), 3.14 (s, 3H), 2.93 (d, J = 4.8 Hz, 3H), 2.73 (s, 3H). | 606 |
| 162 | 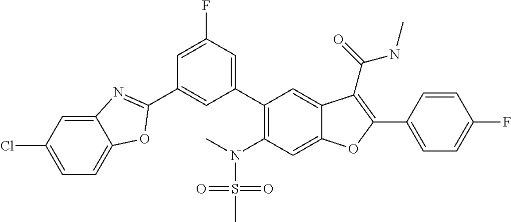 | ¹H-NMR (DMSO, 400 MHz) δ 8.54 (s, 1H), 8.10~8.11 (d, J = 4.0 Hz, 2H), 8.00~8.03 (m, 4H), 7.97~7.98 (d, J = 1.6 Hz, 1H), 7.85~7.87 (d, J = 8.8 Hz, 1H), 7.72 (s, 1H), 7.60~7.62 (d, J = 9.6 Hz, 1H), 7.52~7.53 (d, J = 2.0 Hz, 1H), 3.19 (s, 3H), 3.02 (s, 3H), 2.81~2.82 (d, J = 4.4 Hz, 3H). | 622 |
| 163 | 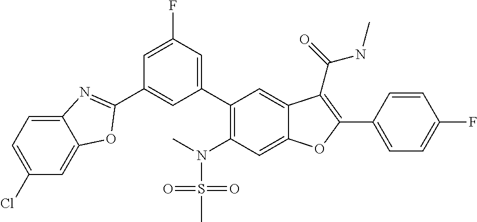 | ¹H-NMR (CDCl₃, 400 MHz) δ 8.15 (s, 1H), 7.94~7.99 (m, 4H), 7.68~7.73 (m, 2H), 7.64 (d, J = 1.6 Hz, 1H), 7.38~7.45 (m, 2H), 7.23~7.27 (m, 2H), 5.88 (d, J = 3.6 Hz, 1H), 3.23 (s, 3H), 3.03 (d, J = 5.2 Hz, 3H), 2.82 (s, 3H). | 622 |
| 164 | 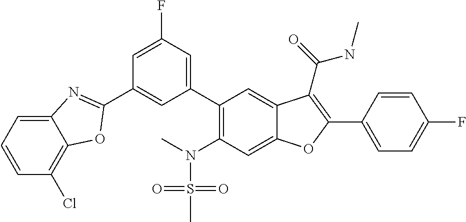 | ¹H-NMR (CDCl₃, 400 MHz) δ 7.93 (s, 1H), 7.89~7.91 (m, 1H), 7.87~7.89 (m, 1H), 7.83 (s, 2H), 7.64~7.68 (m, 2H), 7.36~7.39 (m, 2H), 7.25~7.27 (m, 2H), 7.15~7.18 (m, 1H), 5.84~5.86 (m, 1H), 3.15 (s, 3H), 2.94 (d, J = 4.8 Hz, 3H), 2.70 (s, 3H). | 622 |
| 165 | 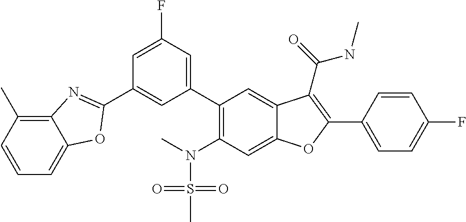 | ¹H-NMR (CDCl₃, 400 MHz) δ 8.64 (d, J = 4.8 Hz, 1H), 8.18 (d, J = 3.2 Hz, 2H), 8.12~8.05 (m, 3H), 7.83 (s, 1H), 7.71 (t, J = 22.0 Hz, 2H), 7.53 (t, J = 18.0 Hz, 2H), 7.46 (t, J = 12.0 Hz, 1H), 3.28 (s, 3H), 3.12 (s, 3H), 2.92 (d, J = 4.8 Hz, 3H), 2.69 (s, 3H). | 602 |
| 166 | 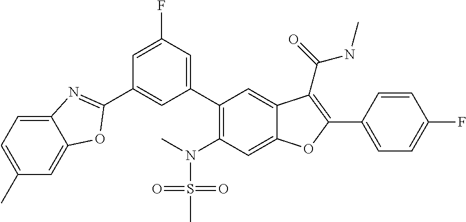 | ¹H-NMR (CDCl₃, 400 MHz) δ 8.06 (s, 1H), 7.86~7.89 (m, 2H), 7.81 (s, 2H), 7.59~7.62 (m, 2H), 7.13~7.37 (m, 5H), 6.02~6.21 (m, 1H), 3.16 (s, 3H), 2.98 (d, J = 4.4 Hz, 3H), 2.76 (s, 3H), 2.49 (s, 3H). | 602 |
| 167 | 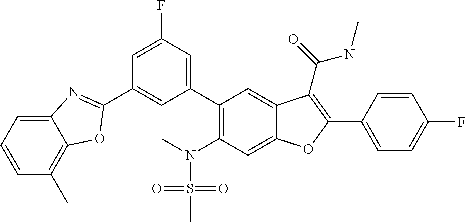 | ¹H-NMR (CDCl₃, 400 MHz) δ 8.08 (s, 1H), 7.84~7.93 (m, 4H), 7.59 (s, 1H), 7.53 (d, J = 8.0 Hz, 1H), 7.31 (d, J = 12.0 Hz, 1H), 7.20~7.22 (m, 1H), 7.10~7.17 (m, 3H), 5.81 (d, J = 4.0 Hz, 1H), 3.13 (s, 3H), 2.93 (d, J = 4.8 Hz, 3H), 2.71 (s, 3H), 2.52 (s, 3H). | 602 |
| 168 | 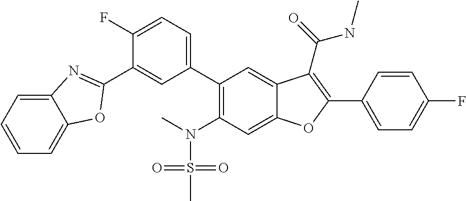 | ¹H-NMR (CDCl₃, 400 MHz) δ 9.73 (s, 1H), 8.45 (d, J = 4.8 Hz, 1H), 8.13~8.15 (m, J = 7.6 Hz, 1H), 7.19~8.13 (m, 2H), 7.92 (s, 1H), 7.69 (s, 1H), 7.60~7.67 (m, 2H), 7.30~7.47 (m, 2H), 7.21~7.26 (m, 2H), 5.31 (s, 1H), 3.22 (s, 3H), 3.03 (d, J = 4.8 Hz, 3H), 2.82 (s, 3H). | 588 |
| 169 | 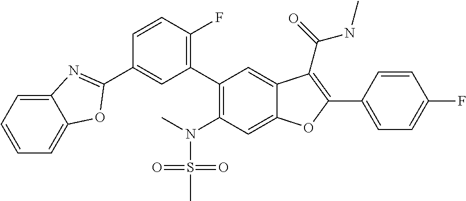 | ¹H-NMR (CDCl₃, 400 MHz) δ 8.21~8.26 (m, 2H), 7.86~7.89 (m, 2H), 7.80 (s, 1H), 7.69 (t, J = 4.4 Hz, 1H), 7.60 (s, 1H), 7.50~7.52 (m, 1H), 7.22~7.31 (m, 3H), 7.13 (t, J = 8.4 Hz, 2H), 6.01 (d, J = 3.6 Hz, 1H), 3.22 (s, 3H), 2.92 (d, J = 4.4 Hz, 3H), 2.55 (s, 3H). | 588 |
| 170 | 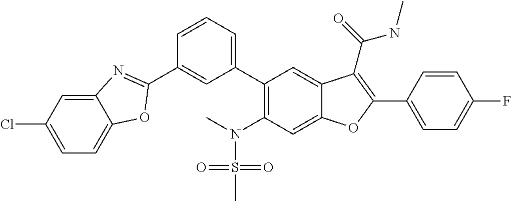 | ¹H-NMR (DMSO, 400 MHz) δ 8.54~8.55 (d, J = 4.4 Hz, 1H), 8.25 (s, 1H), 8.20~8.22 (d, J = 6.4 Hz, 1H), 8.06 (s, 1H), 7.99~8.03 (m, 1H), 7.94~7.95 (d, J = 1.6 Hz, 1H), 7.84~7.86 (d, J = 8.8 Hz, 2H), 7.75 (s, 1H), 7.67~7.73 (m, 1H), 7.49~7.50 (d, J = 2.0 Hz, 1H), 7.47~7.48 (d, J = 2.0 Hz, 1H), 7.40~7.44 (m, 1H), 3.15 (s, 3H), 2.96, (s, 3H), 2.80~2.81 (d, J = 4.4 Hz, 3H). | 604 |
| 171 | 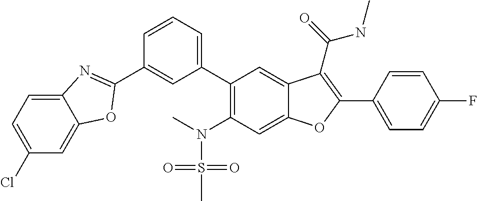 | ¹H-NMR (CDCl₃, 400 MHz) δ 8.33 (s, 1H), 8.28 (d, J = 7.6 Hz, 1H), 7.95~7.98 (m, 2H), 7.90 (s, 1H), 7.71 (d, J = 8.0 Hz, 2H), 7.63~7.67 (m, 3H), 7.38 (d, J = 8.4 Hz, 1H), 7.21~7.26 (m, 2H), 5.98 (s, 1H), 3.21 (s, 3H), 3.02 (d, J = 8.8 Hz, 3H), 2.73 (s, 3H). | 604 |
| 172 | 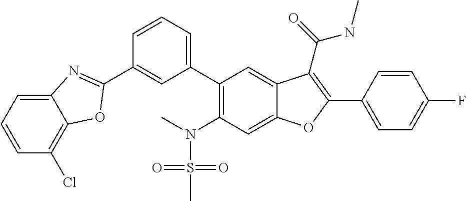 | ¹H-NMR (CDCl₃, 400 MHz) δ 8.36~8.39 (m, 2H), 7.97~7.99 (m, 2H), 7.90 (s, 1H), 7.67~7.69 (m, 3H), 7.37~7.39 (m, 1H), 7.29~7.32 (m, 2H), 7.20~7.23 (m, 2H), 5.84 (t, J = 7.6 Hz, 1H), 3.15 (s, 3H), 2.94 (d, J = 4.8 Hz, 3H), 2.70 (s, 3H). | 604 |
| 173 | 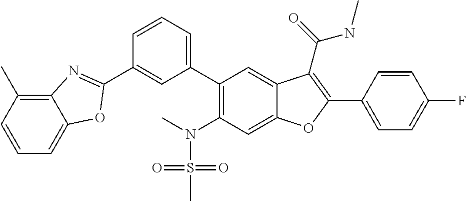 | ¹H-NMR (CDCl₃, 400 MHz) δ 8.55 (d, J = 4.8 Hz, 1H), 8.24 (t, J = 12.0 Hz, 2H), 8.05~8.00 (m, 3H), 7.72 (t, J = 16.4 Hz, 3H), 7.60 (d, J = 8.4 Hz, 1H), 7.44 (t, J = 18 Hz, 1H), 7.34 (t, J = 16.8 Hz, 1H), 7.24 (d, J = 7.6 Hz, 1H), 3.15 (s, 3H), 2.96 (s, 3H), 2.82 (d, J = 4.8 Hz, 3H), 2.60 (s, 3H). | 584 |
| 174 | 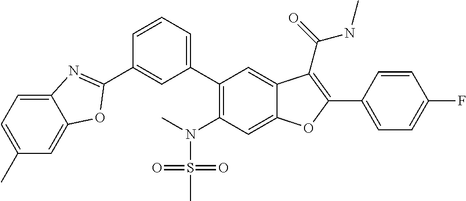 | ¹H-NMR (CDCl₃, 400 MHz) δ 8.22 (s, 1H), 8.17 (d, J = 7.6 Hz, 1H), 7.85~7.88 (m, 2H), 7.77 (s, 1H), 7.50~7.58 (m, 4H), 7.31 (s, 1H), 7.09~7.14 (m, 3H), 5.97~5.98 (m, 1H), 3.10 (s, 3H), 2.91 (d, J = 4.8 Hz, 3H), 2.60 (s, 3H), 2.44 (s, 3H). | 584 |
| 175 | 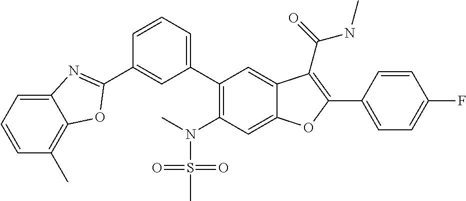 | ¹H-NMR (CDCl₃, 400 MHz) δ 8.24~8.22 (m, 2H), 7.90~7.81 (m, 3H), 7.61~7.52 (m, 4H), 7.21~7.09 (m, 4H), 5.90 (d, J = 4.4 Hz, 1H), 3.12 (s, 3H), 2.94 (d, J = 5.2 Hz, 3H), 2.62 (s, 3H), 2.53 (s, 3H). | 584 |
| 176 | 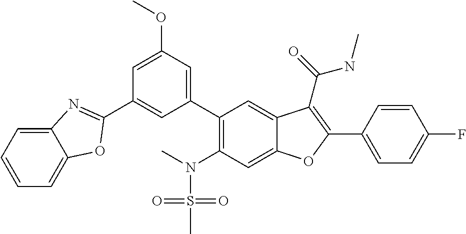 | ¹H-NMR (CDCl₃, 400 MHz) δ 7.79~7.95 (m, 6H), 7.58~7.61 (m, 2H), 7.36~7.38 (m, 2H), 7.16~7.27 (m, 3H), 6.04~6.05 (m, 1H), 3.95 (s, 3H), 3.14 (s, 3H), 2.98 (d, J = 4.4 Hz, 3H), 2.77 (s, 3H). | 600 |
| 177 | 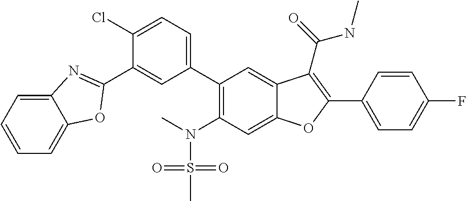 | ¹H-NMR (CDCl₃, 400 MHz) δ 8.18 (d, J = 2.0 Hz, 1H), 7.86~7.90 (m, 2H), 7.82 (s, 1H), 7.78 (d, J = 8.8 Hz, 1H), 7.54~7.61 (m, 4H), 7.32~7.35 (m, 2H), 7.15 (t, J = 8.4 Hz, 2H), 5.70 (br s, 1H), 3.10 (s, 3H), 2.93 (d, J = 5.2 Hz, 3H), 2.76 (s, 3H). | 604 |
| 178 | 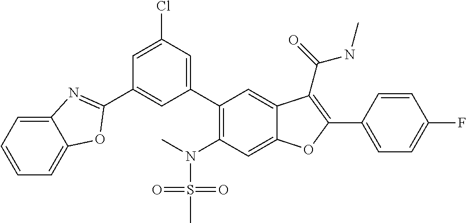 | ¹H-NMR (CDCl₃, 400 MHz) δ 8.21 (s, 1H), 8.18 (s, 1H), 7.87 (t, J = 1.4 Hz, 2H), 7.85 (s, 1H), 7.70~7.81 (m, 1H), 7.54~7.58 (m, 2H), 7.52~7.53 (m, 1H), 7.29~7.33 (m, 2H), 7.12~7.18 (m, 2H), 5.84 (s, 1H), 3.12 (s, 3H), 2.93 (d, J = 4.8 Hz, 3H), 2.72 (s, 3H). | 604 |
| 179 | 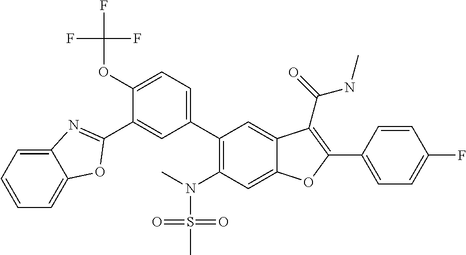 | ¹H-NMR (CDCl₃, 400 MHz) δ 8.31 (s, 1H), 8.43 (s, 1H), 7.84~7.92 (m, 4H), 7.71~7.74 (m, 1H), 7.59 (s, 1H), 7.48~7.50 (m, 2H), 7.29~7.31 (m, 1H), 7.14~7.16 (m, 2H), 5.79~5.80 (m, 1H), 3.12 (s, 3H), 2.93~2.94 (m, 3H), 2.72 (s, 3H). | 654 |
| 180 | 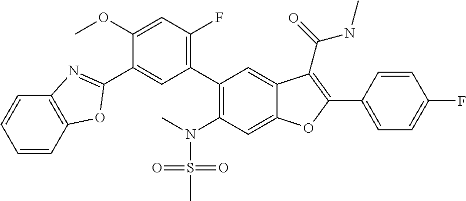 | ¹H-NMR (CDCl₃, 400 MHz) δ 8.21 (d, J = 8.0 Hz, 1H), 7.94~7.97 (m, 2H), 7.86 (s, 1H), 7.81 (d, J = 4.0 Hz, 1H), 7.69 (s, 1H), 7.55~7.58 (m, 1H), 7.34~7.36 (m, 2H), 7.19~7.13 (m, 2H), 6.90 (d, J = 12.0 Hz, 1H), 5.89 (s, 1H), 4.07 (s, 3H), 3.28 (s, 3H), 3.00 (d, J = 8.0 Hz, 3H), 2.67 (s, 3H). | 618 |
| 181 | 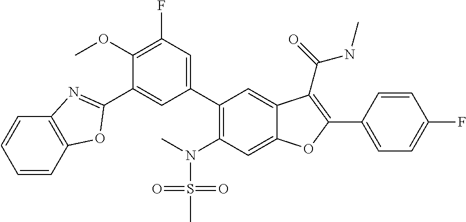 | ¹H-NMR (CDCl₃, 400 MHz) δ 7.92 (s, 1H), 7.85~7.88 (m, 2H), 7.79 (s, 1H), 7.73~7.75 (m, 1H), 7.53~7.56 (m, 2H), 7.37~7.41 (m, 1H), 7.30~7.33 (m, 2H), 7.11~7.15 (m, 2H), 5.87 (d, J = 4.0 Hz, 1H), 4.09 (d, J = 1.6 Hz, 3H), 3.12 (s, 3H), 2.94 (d, J = 4.8 Hz, 3H), 2.77 (s, 3H). | 618 |
| 182 | 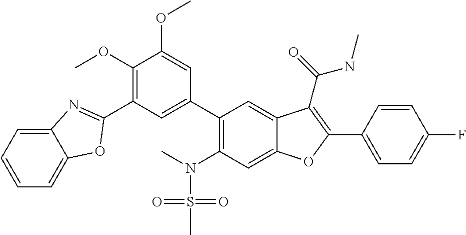 | ¹H-NMR (CDCl₃, 400 MHz) δ 7.89~7.91 (m, 2H), 7.82 (s, 1H), 7.73~7.76 (m, 1H), 7.69 (d, J = 2.0 Hz, 1H), 7.54~7.56 (m, 1H), 7.52 (s, 1H), 7.36 (d, J = 2.0 Hz, 1H), 7.30~7.33 (m, 2H), 7.12~7.18 (m, 2H), 5.77~5.82 (m, 1H), 4.01 (s, 3H), 3.92 (s, 3H), 3.02 (s, 3H), 2.94 (d, J = 4.4 Hz, 3H), 2.87 (s, 3H). | 630 |
| 183 | 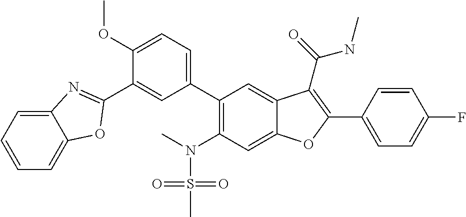 | ¹H-NMR (CDCl₃, 400 MHz) δ 8.43 (d, J = 4.8 Hz, 1H), 8.12~8.14 (m, 1H), 7.96~8.10 (m, 2H), 7.90 (s, 1H), 7.72 (s, 1H), 7.56~7.66 (m, 2H), 7.36~7.46 (m, 2H), 7.20~7.24 (m, 3H), 5.96 (s, 1H), 3.22 (s, 3H), 3.03 (d, J = 4.8 Hz, 3H), 2.82 (s, 3H), 2.73 (s, 3H). | 600 |
| 184 | 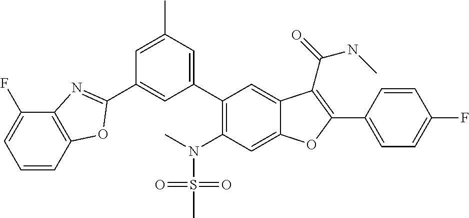 | ¹H-NMR (CDCl₃, 400 MHz) δ 8.18 (s, 2H), 7.97~8.18 (m, 2H), 7.90 (s, 1H), 7.68 (s, 1H), 7.54 (s, 1H), 7.44 (d, J = 7.6 Hz, 1H), 7.34~7.37 (m, 1H), 7.22~7.27 (m, 2H), 7.10~7.15 (m, 1H), 5.93~5.95 (br s, 1H), 3.21 (s, 3H), 3.04 (d, J = 4.8 Hz, 3H), 2.74 (s, 3H), 2.56 (s, 3H). | 602 |
| 185 | 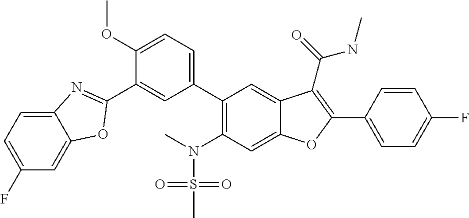 | ¹H-NMR (CDCl₃, 400 MHz) δ 8.04 (s, 1H), 7.84~7.9 (m, 4H), 7.63~7.66 (m, 1H), 7.59 (s, 1H), 7.32~7.34 (m, 1H), 7.25~7.27 (m, 1H), 7.14~7.16 (m, 2H), 7.04~7.09 (m, 1H), 5.76 (s, 1H), 3.14 (s, 3H), 2.94 (m, 3H), 2.72 (s, 3H), 2.10 (s, 3H). | 618 |
| 186 | 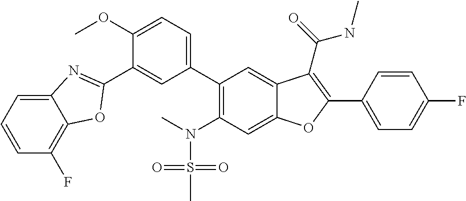 | ¹H-NMR (CDCl₃, 400 MHz) δ 8.15 (d, J = 2.0 Hz, 1H), 7.87~7.89 (m, 2H), 7.80 (s, 1H), 7.63 (d, J = 8.0 Hz, 1H), 7.57 (s, 1H), 7.53 (d, J = 7.6 Hz, 1H), 7.19~7.22 (m, 1H), 7.14 (t, J = 8.8 Hz, 3H), 7.04 (t, J = 8.4 Hz, 1H), 5.81 (s, 1H), 4.03 (s, 3H), 3.11 (s, 3H), 2.93 (d, J = 4.4 Hz, 3H), 2.71 (s, 3H). | 618 |
| 187 | 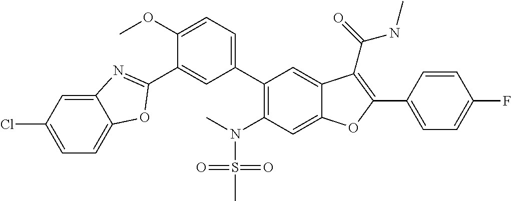 | ¹H-NMR (CDCl₃, 400 MHz) δ 8.11 (s, 1H), 7.83~7.87 (m, 2H), 7.77 (s, 1H), 7.69 (s, 1H), 7.60 (d, J = 3.6 Hz, 1H), 7.52 (s, 1H), 7.42 (d, J = 8.4 Hz, 1H), 7.24 (d, J = 8.4 Hz, 1H), 7.41~7.43 (m, 3H), 5.95~5.96 (d, J = 4.8 Hz, 1H), 4.00 (s, 3H), 3.08 (s, 3H), 2.90~2.91 (d, J = 4.4 Hz, 3H), 2.70 (s, 3H). | 634 |
| 188 | 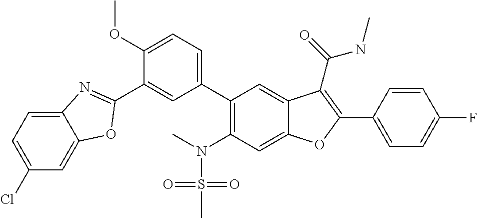 | ¹H-NMR (CDCl₃, 400 MHz) 8.16 (s, 1H), 7.91~7.93 (m, 2H), 7.83 (s, 1H), 7.65~7.71 (m, 2H), 7.59 (d, J = 5.6 Hz, 2H), 7.31 (d, J = 7.6 Hz, 1H), 7.15~7.20 (m, 3H), 5.88 (d, J = 4.0 Hz, 1H), 4.06 (s, 3H), 3.14 (s, 3H), 2.97 (d, J = 4.8 Hz, 3H), 2.76 (s, 3H). | 634 |
| 189 | 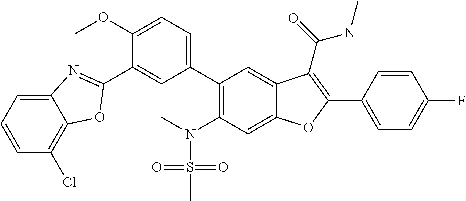 | ¹H-NMR (CDCl₃, 400 MHz) δ 8.17 (s, 1H), 7.90 (m, 2H), 7.80 (s, 1H), 7.65~7.68 (m, 2H), 7.58 (s, 1H), 7.24~7.27 (m, 2H), 7.16~7.19 (m, 3H), 5.84 (t, J = 4.8 Hz, 1H), 4.03 (s, 3H), 3.15 (s, 3H), 2.94 (d, J = 4.8 Hz, 3H), 2.70 (s, 3H). | 634 |
| 190 | 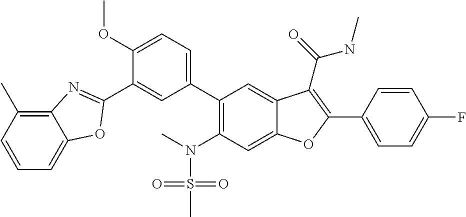 | ¹H-NMR (DMSO, 400 MHz) δ 8.62 (d, J = 4.4 Hz, 1H), 8.15 (d, J = 2.4 Hz, 1H), 8.10 (t, J = 14.4 Hz, 3H), 7.79~7.76 (m, 1H), 7.71 (s, 1H), 7.66 (d, J = 8.0 Hz, 1H), 7.51~7.42 (m, 2H), 7.39 (t, J = 15.6 Hz, 1H), 7.30 (d, J = 7.6 Hz, 1H), 4.06 (s, 3H), 3.21 (s, 3H), 3.09 (s, 3H), 2.89 (d, J = 4.4 Hz, 3H), 2.65 (s, 3H). | 614 |
| 191 | 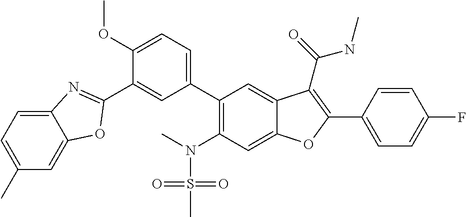 | ¹H-NMR (CDCl₃, 400 MHz) δ 8.12 (d, J = 2.4 Hz, 1H), 7.91~7.87 (m, 2H), 7.78 (s, 1H), 7.65~7.55 (m, 3H), 7.33 (s, 1H), 7.16~7.10 (m, 4H), 5.88 (d, J = 4.8 Hz, 1H), 4.00 (s, 3H), 3.09 (s, 3H), 2.94 (d, J = 4.8 Hz, 3H), 2.70 (s, 3H), 2.44 (s, 3H). | 614 |
| 192 | 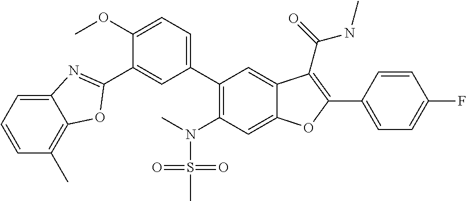 | ¹H-NMR (CDCl₃, 400 MHz) δ 8.22 (s, 1H), 7.93~7.94 (m, 2H), 7.87 (s, 1H), 7.65~7.68 (m, 3H), 7.16~7.25 (m, 5H), 5.91 (d, J = 4.4 Hz, 1H), 4.08 (s, 3H), 3.17 (s, 3H), 3.00 (d, J = 8.0 Hz, 3H), 2.77 (s, 3H), 2.58 (s, 3H). | 614 |
| 193 | 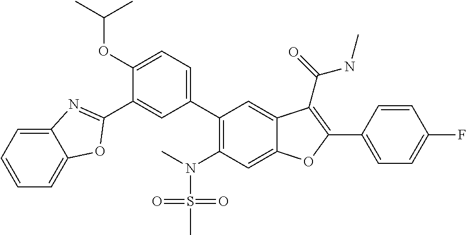 | ¹H-NMR (CDCl₃, 400 MHz) δ 8.05~8.06 (m, 1H), 7.86~7.90 (m, 2H), 7.76~7.78 (m, 2H), 7.53~7.61 (m, 3H), 7.32~7.34 (m, 2H), 7.10~7.14 (m, 3H), 6.01~6.02 (m, 1H), 4.66~4.72 (m, 1H), 3.09 (s, 3H), 2.94 (d, J = 4.8 Hz, 3H), 2.70 (s, 3H), 1.41 (d, J = 6.0 Hz, 6H). | 628 |
| 194 | 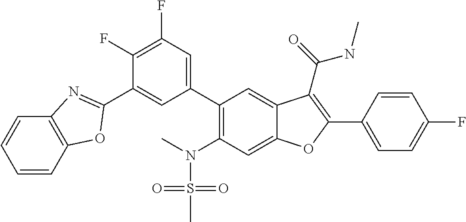 | ¹H-NMR (CDCl₃, 400 MHz) δ 8.04 (s, 1H), 7.77~7.88 (m, 4H), 7.58~7.63 (m, 2H), 7.50 (br s, 1H), 7.39 (br s, 2H), 7.14~7.18 (m, 2H), 6.23 (br s, 1H), 3.17 (s, 3H), 3.00 (br s, 3H), 2.87 (s, 3H). | 606 |
| 195 | 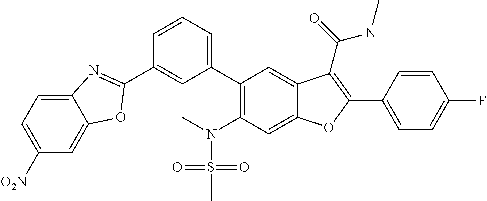 | ¹H-NMR (CDCl₃, 400 MHz) δ 8.44 (s, 1H), 8.32 (s, 1H), 8.24~8.28 (m, 2H), 7.85~7.89 (m, 3H), 7.78~7.80 (d, J = 8.4 Hz, 1H), 7.68~7.70 (m, 1H), 7.58~7.62 (m, 2H), 7.13~7.18 (m, 2H), 5.77~5.78 (m, 1H), 3.03 (s, 3H), 2.92~2.93 (d, J = 4.0 Hz, 3H), 2.68 (s, 3H). | 615 |
| 196 | 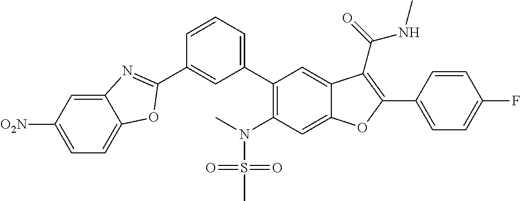 | ¹H-NMR (CDCl₃, 400 MHz) δ 8.71 (s, 1H), 8.33~8.39 (m, 2H), 7.96~7.99 (m, 3H), 7.77 (d, J = 8.0 Hz, 1H), 7.71~7.75 (m, 2H), 7.69 (d, J = 4.8 Hz, 2H), 7.24~7.29 (m, 2H), 5.89 (d, J = 5.2 Hz, 1H), 3.23 (s, 3H), 3.03 (d, J = 5.2 Hz, 3H), 2.78 (s, 3H). | 615 |
| 197 | 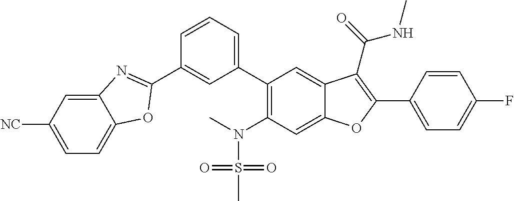 | ¹H-NMR (CDCl₃, 400 MHz) δ 8.27 (s, 1H), 8.21 (d, J = 7.6 Hz, 1H), 8.01 (s, 1H), 7.83~7.88 (m, 2H), 7.67 (s, 1H), 7.64 (d, J = 5.2 Hz, 1H), 7.56~7.61 (s, 4H), 7.14 (t, J = 8.8 Hz, 2H), 5.87 (d, J = 4.4 Hz, 1H), 3.12 (s, 3H), 2.91 (d, J = 5.2 Hz, 3H), 2.67 (s, 3H). | 595 |
| 198 | 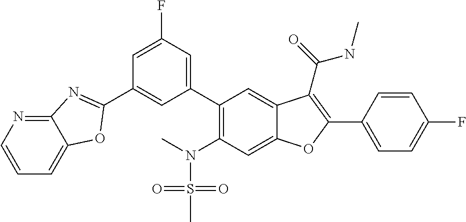 | ¹H-NMR (CDCl₃, 400 MHz) δ 8.73 (s, 1H), 8.24 (s, 1H), 8.11 (d, J = 8.0 Hz, 1H), 8.02 (d, J = 8.4 Hz, 1H), 7.90~7.94 (m, 3H), 7.66 (s, 1H), 7.47~7.62 (m, 2H), 7.23 (t, J = 8.4 Hz, 2H), 6.29 (d, J = 4.0 Hz, 1H), 3.23 (s, 3H), 3.05 (d, J = 4.4 Hz, 3H), 2.87 (s, 3H). | 589 |
| 199 | 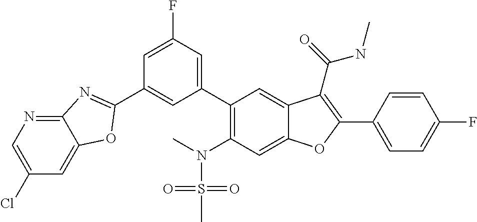 | ¹H-NMR (CDCl₃, 400 MHz) δ 8.44 (s, 1H), 8.23 (s, 2H), 8.17~8.19 (m, 1H), 7.80~7.83 (m, 2H), 7.64 (s, 1H), 7.59~7.61 (m, 1H), 7.41~7.43 (m, 1H), 7.15 (s, 2H), 5.67 (s, 1H), 3.10 (s, 3H), 2.84 (d, J = 4.8 Hz, 3H), 2.55 (s, 3H). | 623 |
| 200 | 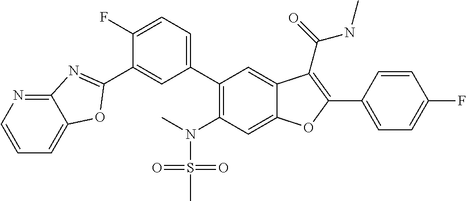 | ¹H-NMR (CDCl₃, 400 MHz) δ 8.62~8.61 (m, 1H), 8.41~8.39 (m, 1H), 7.95~7.91 (m, 3H), 7.88 (s, 1H), 7.74~7.70 (m, 1H), 7.62 (s, 1H), 7.38~7.33 (m, 2H), 7.22~7.18 (m, 2H), 5.86~5.84 (m, 1H), 3.19 (s, 3H), 2.99 (d, J = 4.8 Hz, 3H), 2.81 (s, 3H). | 589 |
| 201 | 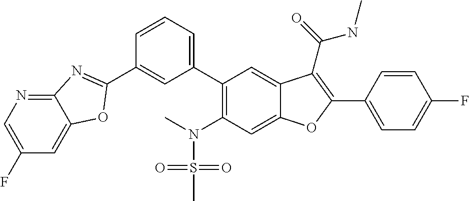 | ¹H-NMR (CDCl₃, 400 MHz) δ 8.43 (s, 1H), 8.31 (s, 1H), 8.25 (d, J = 7.6 Hz, 1H), 7.86~7.90 (m, 2H), 7.84 (s, 1H), 7.66~7.68 (m, 1H), 7.57~7.61 (m, 3H), 7.13~7.18 (m, 2H), 5.81 (br s, 1H), 3.14 (s, 3H), 2.94 (d, J = 4.8 Hz, 3H), 2.65 (s, 3H). | 589 |
| 202 | 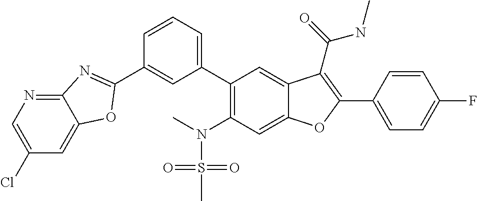 | ¹H-NMR (CDCl₃, 400 MHz) δ 8.49 (s, 1H), 8.32 (s, 2H), 8.27~8.29 (m, 1H), 7.90~7.93 (m, 2H), 7.84 (s, 1H), 7.69~7.72 (m, 1H), 7.61~7.65 (m, 2H), 7.15 (s, 2H), 5.77 (s, 1H), 3.13 (s, 3H), 2.94 (d, J = 4.8 Hz, 3H), 2.65 (s, 3H). | 605 |
| 203 | 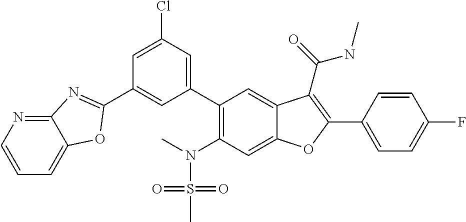 | ¹H-NMR (CDCl₃, 400 MHz) δ 8.57 (d, J = 4.8 Hz, 1H), 8.27 (t, J = 5.2 Hz, 2H), 7.85~7.89 (m, 4H), 7.64 (d, J = 2.0 Hz, 1H), 7.60 (s, 1H), 7.30~7.33 (m, 1H), 7.15 (d, J = 8.8 Hz, 2H), 5.79 (d, J = 4.4 Hz, 1H), 3.15 (s, 3H), 2.94 (d, J = 4.8 Hz, 3H), 2.75 (s, 3H). | 605 |
| 204 | 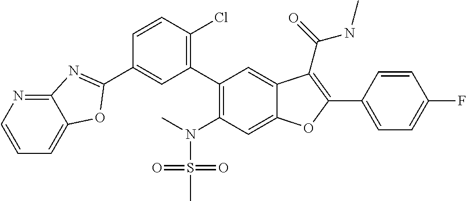 | ¹H-NMR (CDCl₃, 400 MHz) δ 8.63~8.67 (m, 3H), 8.29~8.31 (m, 2H), 7.90~7.92 (m, 2H), 7.56~7.58 (m, 2H), 7.33~7.36 (m, 3H), 3.20 (s, 3H), 3.01 (s, 3H), 2.85 (s, 3H). | 605 |
| 205 | 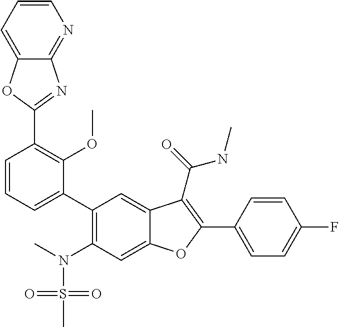 | ¹H-NMR (CDCl₃, 400 MHz) δ 8.75 (d, J = 4.4 Hz, 1H), 8.33~8.35 (m, 1H), 8.17 (d, J = 7.2 Hz, 1H), 7.93~7.97 (m, 3H), 7.70 (d, J = 5.6 Hz, 2H), 7.56~7.59 (m, 1H), 7.46 (t, J = 7.6 Hz, 1H), 7.28 (t, J = 8.8 Hz, 2H), 6.07 (t, J = 4.4 Hz, 1H), 3.63 (s, 3H), 3.28 (s, 3H), 3.06 (d, J = 4.8 Hz, 3H), 2.59 (s, 3H). | 570 |
| 206 | 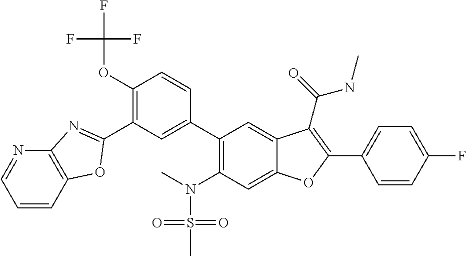 | ¹H-NMR (CDCl₃, 400 MHz) δ 8.56 (s, 1H), 8.43 (s, 1H), 7.84~7.90 (m, 4H), 7.71~7.74 (m, 1H), 7.59 (s, 1H), 7.48~7.50 (m, 1H), 7.29~7.31 (m, 1H), 7.14~7.16 (m, 2H), 5.79~5.81 (m, 1H), 3.14 (s, 3H), 2.93~2.94 (m, 3H), 2.72 (s, 3H). | 655 |
| 207 | 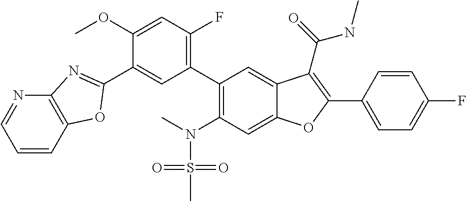 | ¹H-NMR (CDCl₃, 400 MHz) δ 8.49 (d, J = 4.0 Hz, 1H), 8.22 (d, J = 8.0 Hz, 1H), 7.87~7.90 (m, 2H), 7.78~7.81 (m, 2H), 7.59 (s, 1H), 7.39~7.46 (m, 1H), 7.12~7.16 (m, 2H), 6.83 (d, J = 12.0 Hz, 1H), 5.89 (s, 1H), 4.00 (s, 3H), 3.22 (s, 3H), 2.94 (d, J = 8.0 Hz, 3H), 2.63 (s, 3H). | 619 |
| 208 | 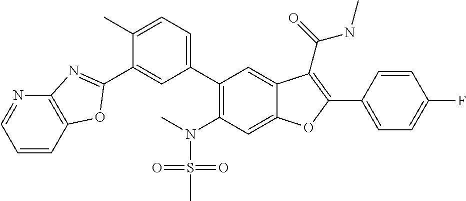 | ¹H-NMR (CDCl₃, 400 MHz) δ 8.55 (d, J = 1.2 Hz, 1H), 8.27 (s, 1H), 7.90~7.81 (m, 4H), 7.57~7.53 (m, 2H), 7.41 (d, J = 8.0 Hz, 1H), 7.31~7.28 (m, 2H), 7.16~7.12 (m, 1H), 5.82 (d, J = 4.4 Hz, 1H), 3.12 (s, 3H), 2.93 (d, J = 4.8 Hz, 3H), 2.85 (s, 3H), 2.68 (s, 3H). | 585 |
| 209 | 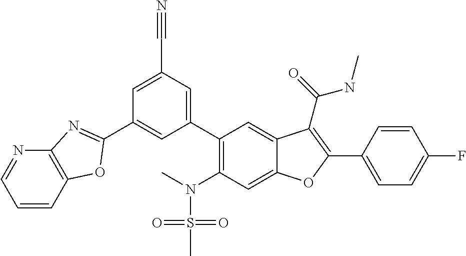 | ¹H-NMR (CDCl₃, 400 MHz) δ 8.56~8.58 (m, 2H), 8.53 (s, 1H), 7.85~7.89 (m, 5H), 7.59 (s, 1H), 7.29~7.33 (m, 1H), 7.17 (t, J = 8.4 Hz, 2H), 5.80 (t, J = 4.0 Hz, 1H), 3.20 (s, 3H), 2.94 (d, J = 4.8 Hz, 3H), 2.78 (s, 3H). | 596 |
| 210 | 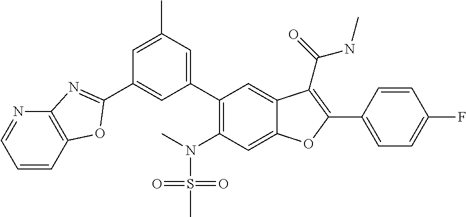 | ¹H-NMR (CDCl₃, 400 MHz) δ 8.59 (d, J = 4.4 Hz, 1H), 8.17 (d, J = 10.0 Hz, 2H), 7.91~7.95 (m, 3H), 7.87 (d, J = 8.0 Hz, 1H), 7.63 (s, 1H), 7.53 (s, 1H), 7.31~7.34 (m, 1H), 7.20 (t, J = 8.4 Hz, 2H), 5.87 (s, 1H), 3.17 (s, 3H), 2.99 (d, J = 4.8 Hz, 3H), 2.71 (s, 3H), 2.53 (s, 3H). | 585 |
| 211 | 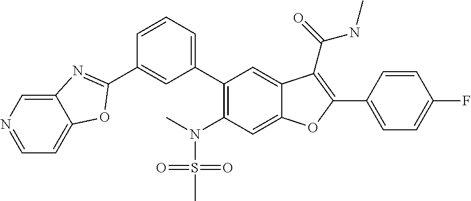 | ¹H-NMR (CDCl₃, 400 MHz) δ 9.25 (s, 1H), 8.82 (d, J = 6.0 Hz, 1H), 8.36 (s, 1H), 8.27 (d, J = 8.0 Hz, 1H), 7.81~7.92 (m, 4H), 7.74~7.76 (d, J = 8.0 Hz, 1H), 7.61~7.65 (m, 1H), 7.56 (s, 1H), 7.14~7.16 (m, 2H), 5.83 (s, 1H), 3.13 (s, 3H), 2.92 (d, J = 4.8 Hz, 3H), 2.68 (s, 3H). | 571 |
| 212 | 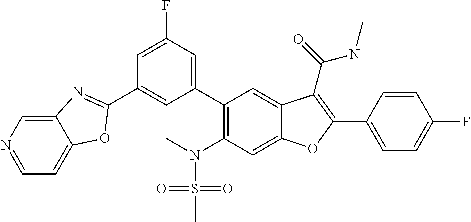 | ¹H-NMR (CDCl₃, 400 MHz) δ 9.21 (s, 1H), 8.75 (d, J = 6.0 Hz, 1H), 8.17 (s, 1H), 7.95 (d, J = 7.6 Hz, 1H), 7.90 (s, 1H), 7.81~7.87 (m, 3H), 7.57 (s, 1H), 7.43~7.46 (m, 1H), 7.15~7.17 (m, 2H), 5.76 (br s, 1H), 3.16 (s, 3H), 2.93 (d, J = 4.8 Hz, 3H), 2.80 (s, 3H). | 589 |
| 213 | 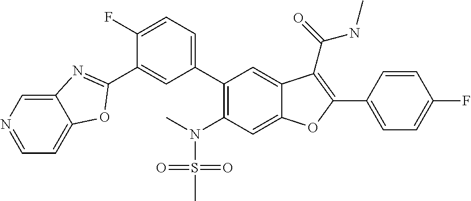 | ¹H-NMR (CDCl₃, 400 MHz) δ 8.95 (s, 1H), 8.43 (d, J = 4.0 Hz, 1H), 8.20 (d, J = 5.6 Hz, 2H), 7.78~7.8 (m, 2H), 7.67 (s, 1H), 7.57~7.58 (m, 2H), 7.52 (s, 1H), 7.05~7.09 (m, 2H), 5.83 (s, 1H), 3.06 (s, 3H), 2.82 (d, J = 4.8 Hz, 3H), 2.75 (s, 3H). | 589 |
| 214 | 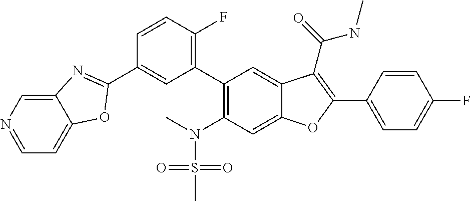 | ¹H-NMR (CDCl₃, 400 MHz) δ 8.53 (d, J = 5.2 Hz, 1H), 8.29~8.23 (m, 2H), 7.89~7.85 (m, 2H), 7.56~7.61 (m, 3H), 7.49~7.47 (m, 1H), 7.27 (t, J = 17.6 Hz, 2H), 7.19 (s, 1H), 5.83 (s, 1H), 3.16 (s, 3H), 2.97 (d, J = 4.8 Hz, 3H), 2.77 (s, 3H). | 589 |
| 215 | 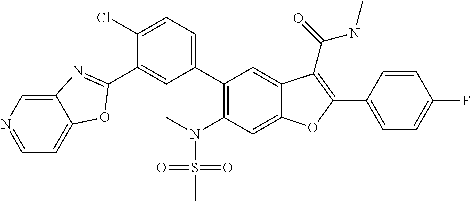 | ¹H-NMR (CDCl₃, 400 MHz) δ 9.45 (s, 1H), 8.90~8.94 (m, 1H), 8.41 (s, 1H), 8.04 (d, J = 5.6 Hz, 1H), 7.99 (s, 1H), 7.91~7.95 (m, 2H), 7.76 (s, 2H), 7.65 (s, 1H), 7.25~7.27 (m, 2H), 5.96 (s, 1H), 3.22 (s, 3H), 3.02 (d, J = 4.8 Hz, 3H), 2.95 (s, 3H). | 605 |
| 216 | 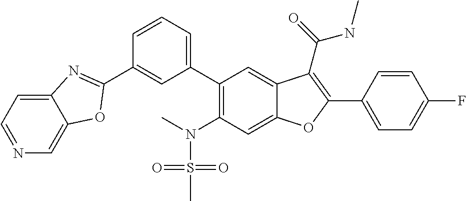 | ¹H-NMR (CDCl₃, 400 MHz) δ 8.59 (s, 1H), 8.43 (d, J = 8.0 Hz, 1H), 8.01 (d, J = 7.2 Hz, 1H), 7.70~7.77 (m, 4H), 7.51~7.57 (m, 4H), 7.28~7.30 (m, 2H), 3.30 (s, 3H), 3.12 (s, 3H), 2.85 (s, 3H). | 571 |
| 217 | 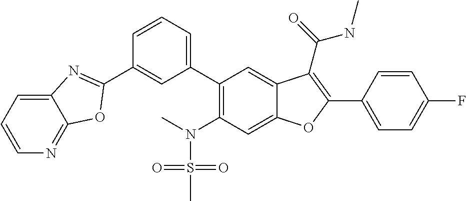 | ¹H-NMR (CDCl₃, 400 MHz) δ 8.35~8.29 (m, 3H), 8.08~8.05 (m, 1H), 7.96~7.92 (m, 2H), 7.86 (s, 1H), 7.71 (d, J = 7.6 Hz, 1H), 7.65~7.61 (m, 2H), 7.37~7.34 (m, 1H), 7.19 (t, J = 8.8 Hz, 2H), 5.88~5.87 (m, 1H), 3.19 (s, 3H), 2.99 (d, J = 4.8 Hz, 3H), 2.68 (s, 3H). | 571 |
| 218 | 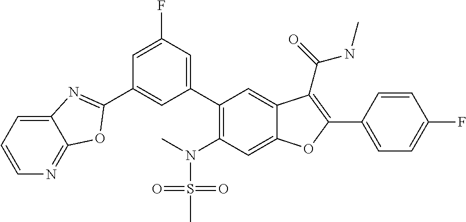 | ¹H-NMR (CDCl₃, 400 MHz) δ 8.42 (d, J = 4.8 Hz, 1H), 8.18 (s, 1H), 8.14 (d, J = 8.0 Hz, 1H), 8.02 (d, J = 4.8 Hz, 1H), 7.97~7.93 (m, 2H), 7.91 (s, 1H), 7.67 (s, 1H), 7.45~7.42 (m, 2H), 7.26~7.21 (m, 2H), 5.93 (s, 1H), 3.25 (s, 3H), 3.04 (d, J = 4.8 Hz, 3H), 2.82 (s, 3H). | 589 |
| 219 | 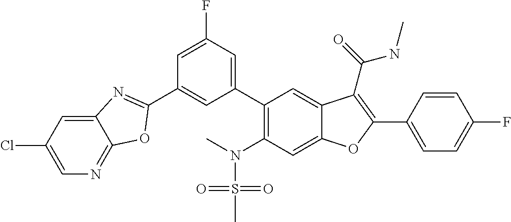 | ¹H-NMR (CDCl₃, 400 MHz) δ 8.27 (s, 1H), 8.08 (s, 1H), 7.99 (s, 1H), 7.83~7.92 (m, 4H), 7.57 (s, 1H), 7.38 (d, J = 8.0 Hz, 1H), 7.13~7.17 (m, 2H), 5.83 (s, 1H), 3.15 (s, 3H), 2.93 (d, J = 4.0 Hz, 3H), 2.73 (s, 3H). | 623 |
| 220 | 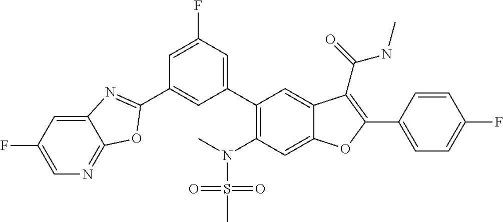 | ¹H-NMR (CDCl₃, 400 MHz) δ 8.38 (s, 1H), 8.34 (d, J = 7.6 Hz, 1H), 8.27 (s, 1H), 7.97~8.00 (m, 2H), 7.93 (s, 1H), 7.81~7.84 (m, 1H), 7.67~7.78 (m, 2H), 7.25 (t, J = 8.4 Hz, 2H), 5.88 (s, 1H), 3.24 (s, 3H), 3.03 (d, J = 4.8 Hz, 3H), 2.74 (s, 3H). | 607 |
| 221 | 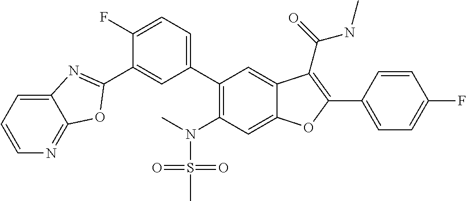 | ¹H-NMR (CDCl₃, 400 MHz) δ 8.36~8.35 (m, 1H), 8.29~8.27 (m, 1H), 8.11~8.09 (m, 1H), 7.93~7.89 (m, 2H), 7.84 (s, 1H), 7.70~7.66 (m, 1H), 7.59 (s, 1H), 7.37~7.32 (m, 2H), 7.18 (t, J = 8.8 Hz, 2H), 5.81 (s, 1H), 3.16 (s, 3H), 2.97 (d, J = 4.8 Hz, 3H), 2.77 (s, 3H). | 589 |
| 222 | 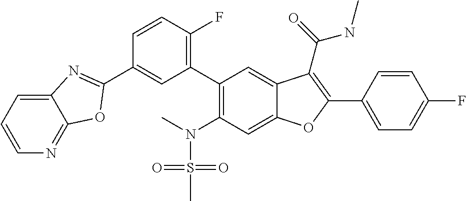 | ¹H-NMR (CDCl₃, 400 MHz) δ 8.25~8.30 (m, 3H), 8.01 (t, J = 6.8 Hz, 1H), 7.88~7.92 (m, 2H), 7.83 (s, 1H), 7.62 (s, 1H), 7.26~7.32 (m, 2H), 7.15 (t, J = 8.4 Hz, 2H), 5.81 (d, J = 4.8 Hz, 1H), 3.23 (s, 3H), 2.95 (d, J = 4.8 Hz, 3H), 2.58 (s, 3H). | 589 |
| 223 | 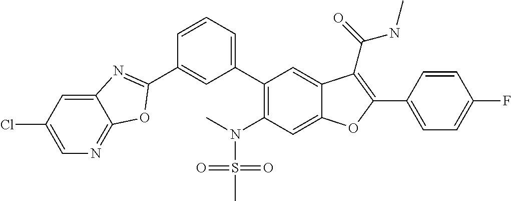 | ¹H-NMR (CDCl₃, 400 MHz) δ 8.22~8.28 (m, 3H), 7.98~7.99 (d, J = 4.0 Hz, 1H), 7.86~7.90 (m, 2H), 7.82 (s, 1H), 7.66~7.68 (d, J = 8.0 Hz, 1H), 7.57~7.61 (m, 2H), 7.13~7.15 (m, 2H), 5.79~7.80 (m, 1H), 3.14 (s, 3H), 2.94 (d, J = 4.0 Hz, 3H), 2.64 (s, 3H). | 605 |
| 224 | 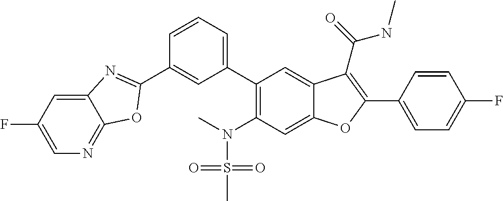 | ¹H-NMR (CDCl₃, 400 MHz) δ 8.38 (s, 1H), 8.34 (d, J = 7.6 Hz, 1H), 8.27 (s, 1H), 7.97~8.00 (m, 2H), 7.93 (s, 1H), 7.67~7.84 (m, 4H), 7.25 (t, J = 8.4 Hz, 2H), 5.88 (br, s, 1H), 3.24 (s, 3H), 3.03 (d, J = 4.8 Hz, 3H), 2.74 (s, 3H). | 589 |
| 225 | 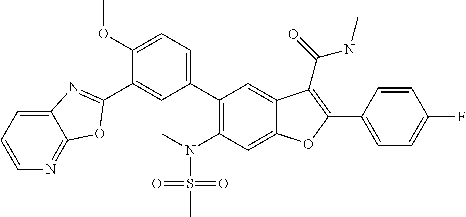 | ¹H-NMR (CDCl₃, 400 MHz) δ 8.37 (d, J = 4.0 Hz, 1H), 8.25 (d, J = 4.0 Hz, 1H), 8.15 (d, J = 8.0 Hz, 1H), 7.94~7.98 (m, 2H), 7.86 (s, 1H), 7.72~7.74 (m, 1H), 7.63 (s, 1H), 7.36~7.39 (m, 1H), 7.19~7.23 (m, 3H), 4.17 (s, 3H), 3.21 (s, 3H), 3.01 (d, J = 4.0 Hz, 3H), 2.78 (s, 3H). | 601 |
| 226 | 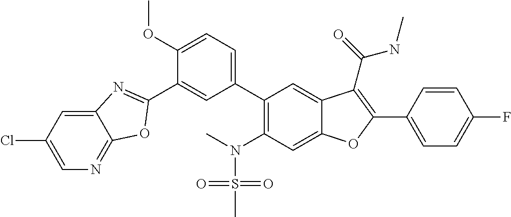 | ¹H-NMR (CDCl₃, 400 MHz) δ 8.23 (s, 1H), 8.16 (d, J = 4.0 Hz, 1H), 8.01 (d, J = 4.0 Hz, 1H), 7.87~7.89 (m, 2H), 7.86 (s, 1H), 7.65~7.79 (m, 1H), 7.55 (s, 1H), 7.12~7.16 (m, 3H), 5.81 (s, 1H), 4.03 (s, 3H), 3.12 (s, 3H), 2.93 (d, J = 4.0 Hz, 3H), 2.72 (s, 3H). | 635 |
| 227 | 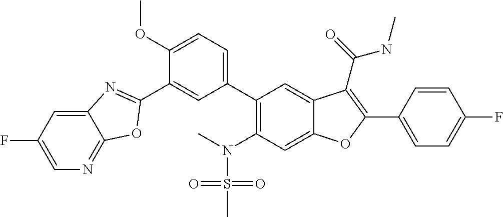 | ¹H-NMR (CDCl₃, 400 MHz) δ 8.17 (d, J = 8.4 Hz, 2H), 7.77~7.86 (m, 4H), 7.68 (d, J = 8.0 Hz, 1H), 7.56 (s, 1H), 7.13~7.17 (m, 3H), 5.82 (s, 1H), 4.03 (s, 3H), 3.13 (s, 3H), 2.93 (d, J = 4.8 Hz, 3H), 2.72 (s, 3H). | 619 |
| 228 | 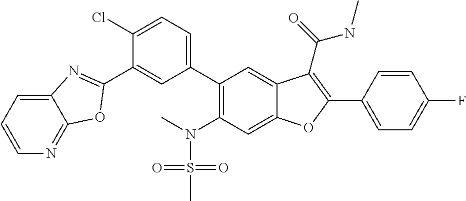 | ¹H-NMR (CDCl₃, 400 MHz) δ 8.43 (d, J = 3.2 Hz, 1H), 8.35 (t, J = 1.2 Hz, 1H), 8.21 (d, J = 2.0 Hz, 1H), 8.06 (s, 1H), 8.00~7.96 (m, 1H), 7.79 (d, J = 8.4 Hz, 2H), 7.72~7.67 (m, 2H), 7.55~7.52 (m, 1H), 7.39 (t, J = 17.6 Hz, 2H), 3.16 (s, 3H), 2.98 (s, 3H), 2.78 (d, J = 4.4 Hz, 3H). | 605 |
| 229 | 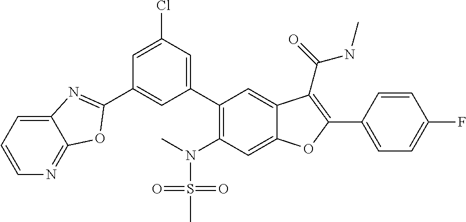 | ¹H-NMR (CDCl₃, 400 MHz) δ 8.42 (d, J = 3.6 Hz, 1H), 8.34 (s, 1H), 8.29 (s, 2H), 8.13 (d, J = 7.6 Hz, 1H), 7.96~7.99 (m, 1H), 7.93 (s, 1H), 7.70 (d, J = 10.0 Hz, 2H), 7.41~7.44 (m, 1H), 7.25 (t, J = 8.4 Hz, 2H), 5.90 (s, 1H), 3.25 (s, 3H), 3.04 (d, J = 4.8 Hz, 3H), 2.83 (s, 3H). | 605 |
| 230 | 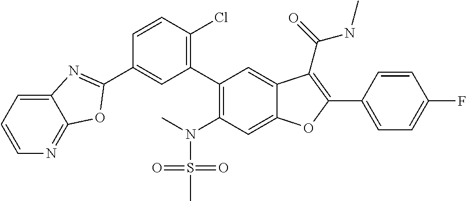 | ¹H-NMR (CDCl₃, 400 MHz) δ 8.30 (s, 2H), 8.17~8.20 (m, 1H), 8.00 (d, J = 7.6 Hz, 1H), 7.89~7.93 (m, 2H), 7.75 (s, 1H), 7.61 (d, J = 6.4 Hz, 2H), 7.29~7.32 (m, 1H), 7.15 (t, J = 8.8 Hz, 2H), 5.81 (s, 1H), 3.22 (s, 3H), 2.93 (d, J = 4.8 Hz, 3H), 2.57 (s, 3H). | 605 |
| 231 | 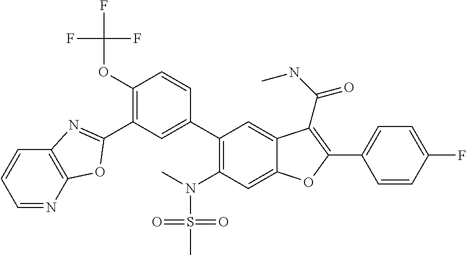 | ¹H-NMR (CDCl₃, 400 MHz) δ 8.56 (s, 1H), 8.43 (s, 1H), 7.84~7.90 (m, 4H), 7.71~7.74 (m, 1H), 7.59 (s, 1H), 7.48~7.50 (m, 1H), 7.29~7.31 (m, 1H), 7.14~7.16 (m, 2H), 5.79~5.80 (m, 1H), 3.14 (s, 3H), 2.93~2.94 (m, 3H), 2.72 (s, 3H). | 655 |
| 232 | 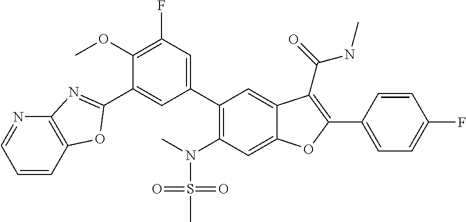 | ¹H-NMR (CDCl₃, 400 MHz) δ 8.61 (d, J = 4.4 Hz, 1H), 8.06 (s, 1H), 7.90~7.96 (m, 3H), 7.85 (s, 1H), 7.61 (s, 1H), 7.50 (d, J = 3.6 Hz, 1H), 7.35~7.38 (m, 1H), 7.17~7.21 (m, 2H), 5.93 (s, 1H), 4.12 (d, J = 1.2 Hz, 3H), 3.19 (s, 3H), 2.99 (d, J = 4.4 Hz, 3H), 2.85 (s, 3H). | 619 |
| 233 | 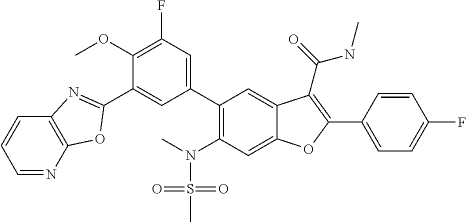 | ¹H-NMR (CDCl₃, 400 MHz) δ 8.34 (d, J = 4.0 Hz, 1H), 8.05~8.32 (m, 1H), 7.93 (s, 1H), 7.86~7.89 (m, 2H), 7.80 (s, 1H), 7.57 (s, 1H), 7.42~7.45 (m, 1H), 7.30~7.33 (m, 1H), 7.12~7.16 (m, 2H), 5.89 (s, 1H), 4.14 (s, 3H), 3.15 (s, 3H), 2.94 (d, J = 4.0 Hz, 3H), 2.78 (s, 3H). | 619 |
| 234 | 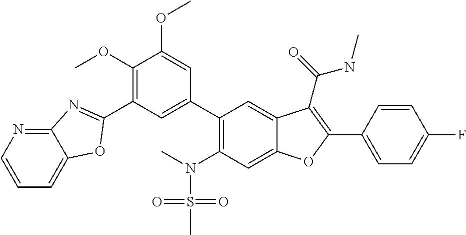 | ¹H-NMR (CDCl₃, 400 MHz) δ 8.58~8.61 (m, 1H), 7.89~7.98 (m, 4H), 7.84 (d, J = 2.0 Hz, 1H), 7.59 (s, 1H), 7.46 (d, J = 2.0 Hz, 1H), 7.31~7.35 (m, 1H), 7.19~7.23 (m, 2H), 5.94~5.95 (m, 1H), 4.11 (s, 3H), 4.00 (s, 3H), 3.11 (s, 3H), 3.02 (d, J = 4.8 Hz, 3H), 2.94 (s, 3H). | 631 |
| 235 | 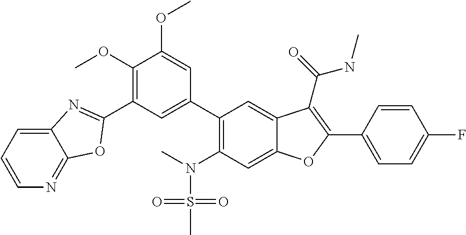 | ¹H-NMR (CDCl₃, 400 MHz) δ 8.30~8.32 (m, 1H), 8.03~8.06 (m, 1H), 7.89~7.92 (m, 2H), 7.81 (s, 1H), 7.71 (d, J = 2.0 Hz, 1H), 7.52 (s, 1H), 7.39 (d, J = 2.0 Hz, 1H), 7.29~7.32 (m, 1H), 7.12~7.16 (m, 2H), 5.79~5.81 (m, 1H), 4.06 (s, 3H), 3.93 (s, 3H), 3.04 (s, 3H), 2.94 (d, J = 4.8 Hz, 3H), 2.87 (s, 3H). | 631 |
| 236 | 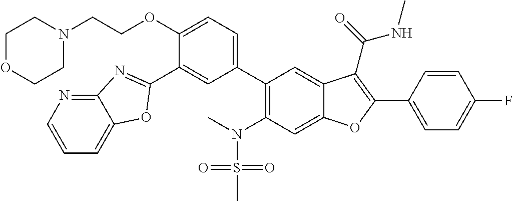 | ¹H-NMR (CDCl₃, 400 MHz) δ 8.54 (d, J = 4.4 Hz, 1H), 8.27 (s, 1H), 7.83~7.93 (m, 4H), 7.65 (d, J = 8.0 Hz, 1H), 7.59 (s, 1H), 7.28~7.31 (m, 1H), 7.14~7.19 (m, 3H), 6.25 (br s, 1H), 4.46 (br s, 2H), 3.82 (br s, 4H), 3.15 (br s, 5H), 2.99 (d, J = 4.8 Hz, 3H), 2.93 (br s, 4H), 2.81 (s, 3H). | 700 |
| 237 | 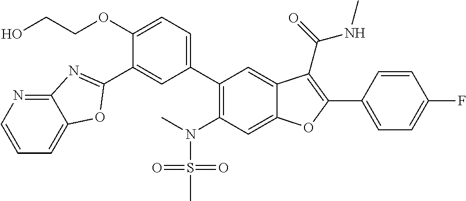 | ¹H-NMR (CDCl₃, 400 MHz) δ 8.55 (br s, 1H), 8.22 (s, 1H), 7.83~7.95 (m, 4H), 7.67 (d, J = 8.0 Hz, 1H), 7.60 (s, 1H), 7.27~7.29 (m, 1H), 7.17~7.22 (m, 3H), 6.14 (br s, 1H), 4.36 (br s, 2H), 4.04 (br s, 2H), 3.14 (s, 3H), 3.00 (d, J = 4.8 Hz, 3H), 2.81 (s, 3H). | 631 |
| 238 | 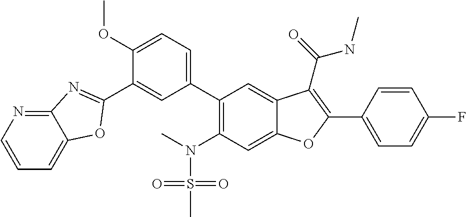 | ¹H-NMR (CDCl₃, 400 MHz) δ 8.53 (s, 1H), 8.25 (s, 1H), 7.84~7.91 (m, 3H), 7.80 (s, 1H), 7.66 (d, J = 8.0 Hz, 1H), 7.56 (s, 1H), 7.25 (t, J = 5.2 Hz, 1H), 7.14 (t, J = 8.8 Hz, 3H), 5.83 (s, 1H), 4.02 (s, 3H), 3.12 (s, 3H), 2.94 (d, J = 4.8 Hz, 3H), 2.73 (s, 3H). | 601 |
| 239 | 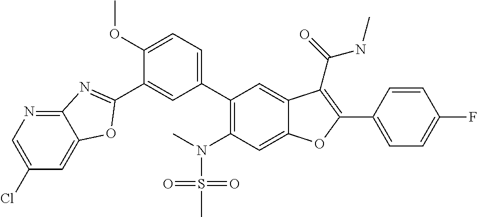 | ¹H-NMR (CDCl₃, 400 MHz) δ 8.85 (s, 1H), 8.54~8.57 (m, 2H), 8.13 (s, 1H), 8.02~8.04 (m, 3H), 7.75~7.78 (m, 1H), 7.62 (s, 1H), 7.42~7.45 (m, 2H), 5.80 (br s, 1H), 4.00 (s3H), 3.15 (s, 3H), 2.99 (s, 3H), 2.80 (d, J = 4.8 Hz, 3H). | 635 |
| 240 | 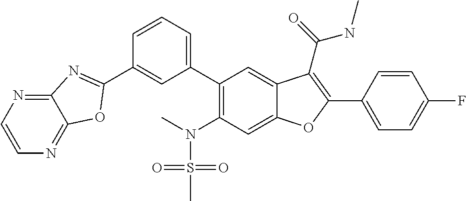 | ¹H-NMR (CDCl₃, 400 MHz) δ 8.52 (d, J = 2.8 Hz, 1H), 8.38 (s, 1H), 8.33 (d, J = 7.6 Hz, 1H), 8.26 (d, J = 2.4 Hz, 1H), 7.87~7.91 (m, 2H), 7.84 (s, 1H), 7.73 (d, J = 8.0 Hz, 1H), 7.59~7.64 (m, 2H), 7.15 (t, J = 8.4 Hz, 2H), 5.78 (br, s, 1H), 3.16 (s, 3H), 2.93 (d, J = 5.2 Hz, 3H), 2.66 (s, 3H). | 572 |
| 241 | 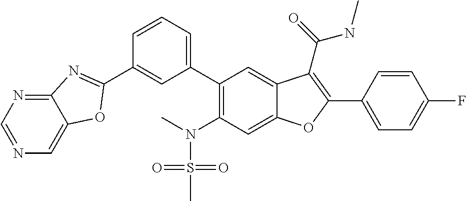 | ¹H-NMR (CDCl₃, 400 MHz) δ 9.14 (s, 1H), 8.97 (s, 1H), 8.40 (s, 1H), 8.30~8.34 (m, 1H), 7.84~7.89 (m, 3H), 7.87 (d, J = 7.6 Hz, 1H), 7.63 (t, J = 7.6 Hz, 1H), 7.58 (s, 1H), 7.16 (t, J = 8.0 Hz, 2H), 5.80 (br, s, 1H), 3.14 (s, 3H), 2.93 (d, J = 4.8 Hz, 3H), 2.69 (s, 3H). | 572 |
| 242 | 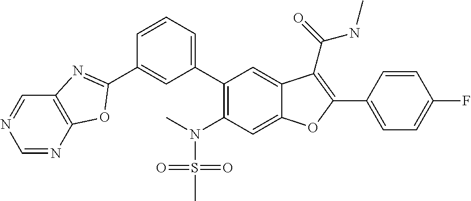 | ¹H-NMR (CDCl₃, 400 MHz) δ 9.09 (s, 1H), 8.98 (s, 1H), 8.31 (s, 2H), 8.27~8.29 (m, 1H), 7.88~7.89 (m, 2H), 7.84 (s, 1H), 7.72~7.74 (m, 1H), 7.63~7.65 (m, 1H), 7.58 (s, 1H), 7.16~7.19 (m, 1H), 5.77 (s, 1H), 3.15 (s, 3H), 2.94 (d, J = 4.8 Hz, 3H), 2.67 (s, 3H). | 572 |
| 243 | 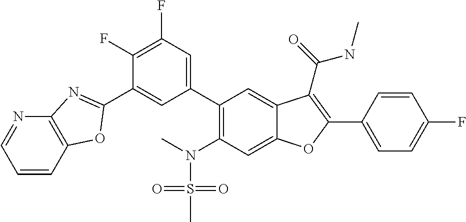 | ¹H-NMR (DMSO, 400 MHz) δ 8.62 (s, 1H), 8.56 (d, J = 4.8 Hz, 1H), 8.32~8.36 (m, 1H), 8.12 (s, 2H), 8.00~8.04 (m, 2H), 7.85~7.90 (m, 1H), 7.75 (s, 1H), 7.53~7.57 (m, 1H), 7.40~7.43 (m, 2H), 3.22 (s, 3H), 3.05 (s, 3H), 2.83 (d, J = 4.4 Hz, 3H). | 607 |
| 244 | 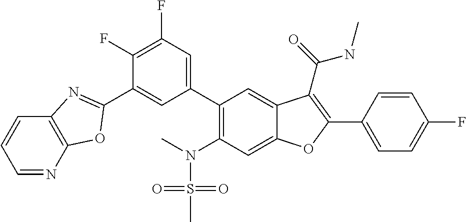 | ¹H-NMR (CDCl₃, 400 MHz) δ 8.42 (s, 1H), 8.17 (d, J = 8.0 Hz, 1H), 8.10 (d, J = 4.8 Hz, 1H), 7.92~7.96 (m, 2H), 7.90 (s, 1H), 7.64 (s, 1H), 7.56~7.61 (m, 1H), 7.40~7.44 (m, 1H), 7.20~7.23 (m, 2H), 5.86 (br s, 1H), 3.22 (s, 3H), 3.01 (d, J = 4.8 Hz, 3H), 2.88 (s, 3H). | 607 |
| 245 | 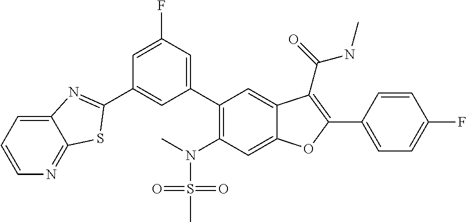 | ¹H-NMR (CDCl₃, 400 MHz) δ 8.54~8.55 (m, 1H), 8.24~8.26 (m, 1H), 7.80~7.91 (m, 5H), 7.58 (s, 1H), 7.41~7.44 (m, 1H), 7.30 (d, J = 8.4 Hz, 1H), 7.13~7.17 (m, 2H), 5.83~5.84 (m, 1H), 3.13 (s, 3H), 2.94 (d, J = 4.8 Hz, 3H), 2.74 (s, 3H). | 605 |
| 246 | 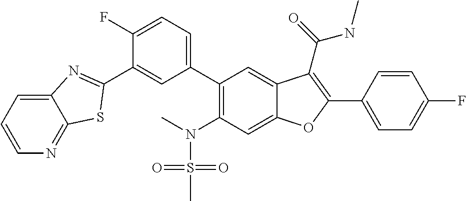 | ¹H-NMR (CDCl₃, 400 MHz) δ 9.51 (s, 1H), 8.75 (s, 1H), 8.57 (dd, J = 2.0 Hz, J = 2.0 Hz, 1H), 8.36 (d, J = 5.6 Hz, 1H), 7.93 (s, 1H), 7.87~7.90 (m, 2H), 7.76~7.80 (m, 1H), 7.60 (s, 1H), 7.37 (m, 1H), 7.22~7.18 (m, 2H), 5.96 (s, 1H), 3.21 (s, 3H), 2.98 (d, J = 4.8 Hz, 3H), 2.88 (s, 3H). | 605 |
| 247 | 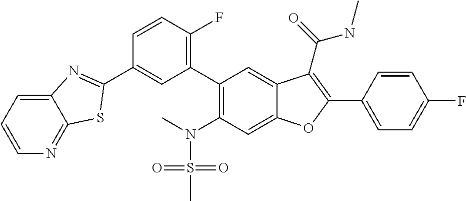 | ¹H-NMR (CDCl₃, 400 MHz) δ 8.55~8.62 (m, 1H), 8.29~8.32 (m, 1H), 8.15~8.20 (m, 2H), 7.94~7.98 (m, 2H), 7.91 (s, 1H), 7.68 (s, 1H), 7.46~7.50 (m, 1H), 7.29~7.34 (m, 1H), 7.20~7.27 (m, 2H), 5.93 (br s, 1H), 3.29 (s, 3H), 3.00 (d, J = 4.8 Hz, 3H), 2.67 (s, 3H). | 605 |
| 248 | 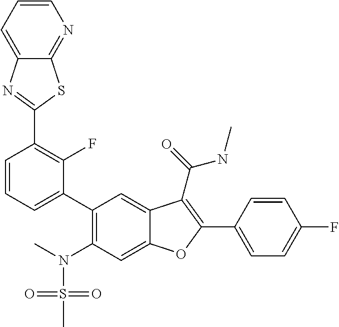 | ¹H NMR: (CDCl₃, 400 MHz) δ 9.78 (s, 1H), 7.84~7.88 (m, 2H), 7.81 (s, 1H), 7.56 (s, 1H), 7.48~7.50 (m, 2H), 7.17~7.31 (m, 3H), 7.15 (t, J = 8.8 Hz, 2H), 5.70 (s, 1H), 3.28 (s, 3H), 2.93 (d, J = 4.8 Hz, 3H), 2.69 (s, 3H). | 605 |
| 249 | 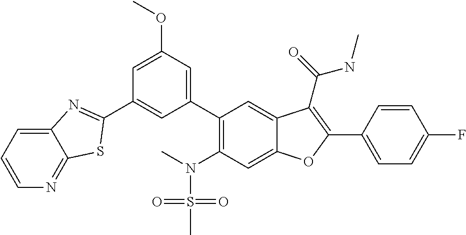 | ¹H-NMR (CDCl₃, 400 MHz) δ 8.63~8.64 (m, 1H), 8.36 (d, J = 8.4 Hz, 1H), 7.98~7.80 (m, 2H), 7.96 (s, 1H), 7.91 (s, 1H), 7.72 (s, 1H), 7.66 (s, 1H), 7.51~7.55 (m, 1H), 7.22~7.29 (m, 3H), 5.96~5.97 (m, 1H), 4.00 (s, 3H), 3.19 (s, 3H), 3.02 (d, J = 4.8 Hz, 3H), 2.84 (s, 3H). | 617 |
| 250 | 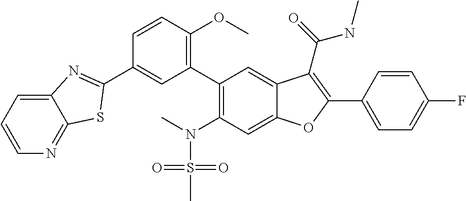 | ¹H-NMR (CDCl₃, 400 MHz) δ 9.66 (s, 1H), 9.36 (d, J = 8.4 Hz, 1H), 9.28~9.26 (m, 1H), 9.18 (d, J = 2.0 Hz, 1H), 9.11~9.08 (m, 2H), 8.93 (s, 1H), 8.77 (s, 1H), 8.57~8.54 (m, 1H), 8.33 (t, J = 8.8 Hz, 1H), 8.23 (d, J = 8.8 Hz, 1H), 7.01 (d, J = 3.6 Hz, 1H), 4.99 (s, 3H), 4.32 (s, 3H), 4.12 (d, J = 4.8 Hz, 3H), 3.73 (s, 3H). | 617 |
| 251 | 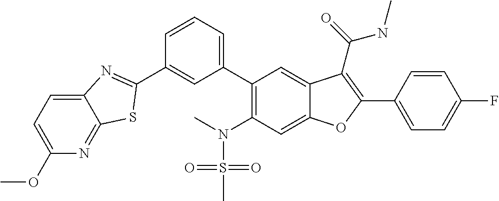 | ¹H-NMR (CDCl₃, 400 MHz) δ 8.18 (d, J = 8.0 Hz, 2H), 8.06 (d, J = 8.0 Hz, 1H), 7.95~7.99 (m, 2H), 7.90 (s, 1H), 7.52~7.65 (m, 3H), 7.19~7.24 (m, 2H), 6.89 (d, J = 8.0 Hz, 1H), 5.96 (s, 1H), 4.04 (s, 3H), 3.19 (s, 3H), 3.01 (d, J = 4.0 Hz, 3H), 2.72 (s, 3H). | 617 |
| 252 | 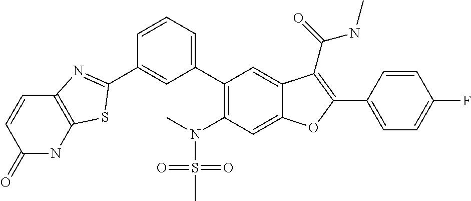 | ¹H-NMR (CDCl₃, 400 MHz) δ 8.42 (d, J = 4.0 Hz, 1H), 8.09 (s, 1H), 7.91~7.96 (m, 4H), 7.64 (s, 2H), 7.39 (s, 1H), 7.34~7.36 (m, 2H), 6.80 (s, 1H), 4.80 (d, J = 4.0 Hz, 1H), 3.24 (s, 3H), 2.76 (d, J = 4.0 Hz, 3H), 2.40 (s, 3H). | 603 |
| 253 | 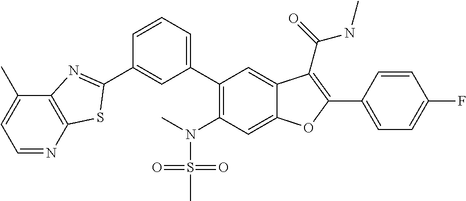 | ¹H-NMR (CDCl₃, 400 MHz) δ 8.45 (d, J = 4.8 Hz, 1H), 8.13~8.15 (m, J = 7.6 Hz, 1H), 7.99~8.13 (m, 2H), 7.92 (s, 1H), 7.69 (s, 1H), 7.60~7.67 (m, 2H), 7.40~7.57 (m, 1H), 7.21~7.26 (m, 3H), 5.31 (s, 1H), 3.22 (s, 3H), 3.03 (d, J = 4.8 Hz, 3H), 2.82 (s, 3H), 2.73 (s, 3H). | 601 |
| 254 | 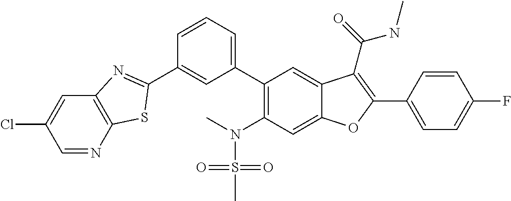 | ¹H-NMR (CDCl₃, 400 MHz) δ 8.53 (s, 1H), 8.24 (s, 1H), 8.17 (s, 1H), 8.08 (d, J = 8.0 Hz, 1H), 7.93 (dd, J = 8.0 Hz, 2H), 7.88 (s, 1H), 7.62~7.67 (m, 3H), 7.21 (t, J = 4.0 Hz, 2H), 5.86 (d, J = 4.0 Hz, 1H), 3.16 (s, 3H), 2.97 (d, J = 4.0 Hz, 3H), 2.72 (s, 3H). | 621 |
| 255 | 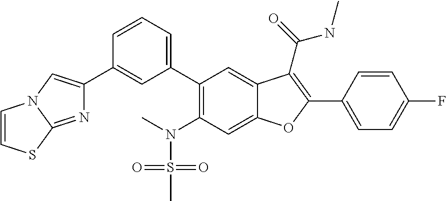 | ¹H-NMR (CDCl₃, 400 MHz) δ 7.90~7.93 (m, 2H), 7.88 (s, 1H), 7.82 (d, J = 7.6 Hz, 1H), 7.77 (s, 2H), 7.55 (s, 1H), 7.39~7.44 (m, 2H), 7.30 (d, J = 7.2 Hz, 1H), 7.13 (t, J = 8.4 Hz, 2H), 6.79 (d, J = 4.4 Hz, 1H), 5.86 (s, 1H), 3.07 (s, 3H), 2.94 (d, J = 4.8 Hz, 3H), 2.61 (s, 3H). | 575 |
| 256 | 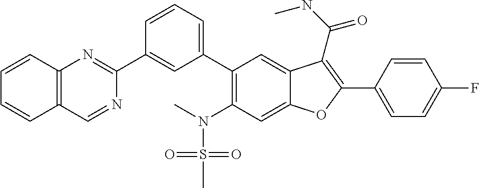 | ¹H-NMR (CDCl₃, 400 MHz) δ 9.53 (s, 1H), 8.69 (s, 1H), 8.63~8.65 (m, 1H), 8.09~8.12 (m, 1H), 7.93~7.97 (m, 4H), 7.88 (s, 1H), 7.60~7.68 (m, 4H), 7.12~7.20 (m, 2H), 5.91 (br s, 1H), 3.20 (s, 3H), 3.02 (s, 3H), 2.62 (s, 3H). | 502 |
| 257 | 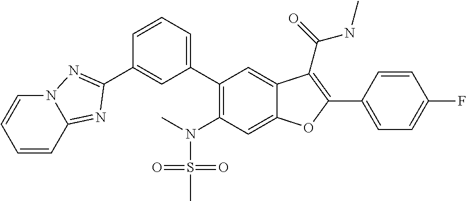 | ¹H-NMR (CDCl₃, 400 MHz) δ 8.60 (d, J = 6.8 Hz, 1H), 8.28 (s, 1H), 8.22 (d, J = 7.2 Hz, 1H), 7.99 (d, J = 9.2 Hz, 1H), 7.91~7.94 (m, 2H), 7.80 (s, 1H), 7.68 (d, J = 7.6 Hz, 1H), 7.57~7.64 (m, 3H), 7.14 (t, J = 8.4 Hz, 3H), 6.18 (s, 1H), 3.13 (s, 3H), 2.94 (d, J = 4.4 Hz, 3H), 2.60 (s, 3H). | 570 |
| 258 | 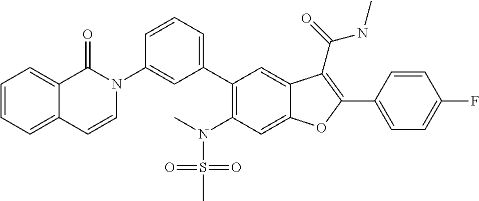 | ¹H-NMR (CDCl₃, 400 MHz) δ 8.38 (d, J = 8.0 Hz, 1H), 7.87~7.90 (m, 2H), 7.77 (s, 1H), 7.61 J = 8.0 Hz, 1H), 7.39~7.54 (m, 8H), 7.14 (t, J = 8.0 Hz, 2H), 6.54 (d, J = 8.0 Hz, 1H), 5.81 (s, 1H), 3.15 (s, 3H), 2.93 (d, J = 4.0 Hz, 3H), 2.67 (s, 3H). | 596 |
| 259 | 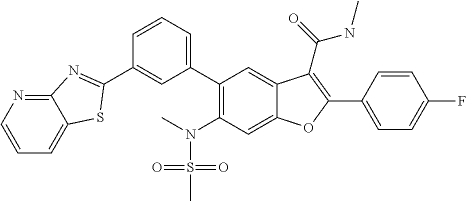 | ¹H-NMR (CDCl₃, 400 MHz) δ 8.71 (d, J = 4.0 Hz, 1H), 8.28 (s, 2H), 8.16 (d, J = 8.0 Hz, 1H), 7.88~7.92 (m, 2H), 7.82 (s, 1H), 7.63~7.54 (m, 3H), 7.30~7.34 (m, 1H), 7.13~7.17 (m, 2H), 5.84~5.85 (s, 1H), 3.12 (s, 3H), 2.94 (d, J = 4.0 Hz, 3H), 2.68 (s, 3H). | 587 |
| 260 | 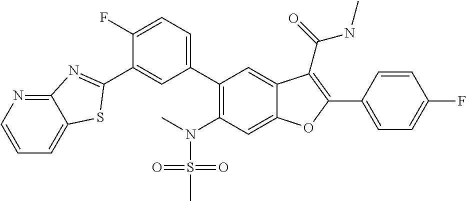 | ¹H-NMR (CDCl₃, 400 MHz) δ 8.83~8.84 (m, 1H), 8.67~8.70 (m, 1H), 8.43~8.45 (m, 1H), 7.95~7.99 (m, 2H), 7.89 (s, 1H), 7.73~7.76 (m, 2H), 7.35~7.47 (m, 2H), 7.19~7.24 (m, 2H), 5.96~5.97 (m, 1H), 3.24 (s, 3H), 3.02 (d, J = 4.4 Hz, 3H), 2.84 (s, 3H). | 605 |
| 261 | 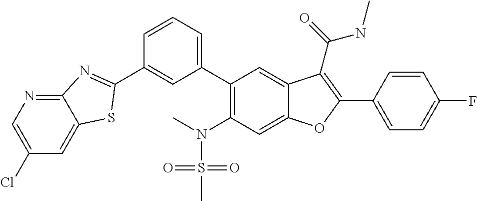 | ¹H-NMR (CDCl₃, 400 MHz) δ 8.60 (d, J = 4.0 Hz, 1H), 8.21 (s, 1H), 8.18 (s, 1H), 8.13 (d, J = 8.0 Hz, 1H), 7.90 (dd, J = 4.0 Hz, 2H), 7.81 (s, 1H), 7.53~7.62 (m, 3H), 7.15 (t, J = 8.0 Hz, 2H), 5.85 (s, 1H), 3.10 (s, 3H), 2.93 (d, J = 4.0 Hz, 3H), 2.69 (s, 3H). | 621 |
| 262 | 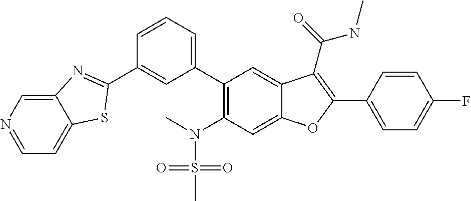 | ¹H-NMR (CDCl₃, 400 MHz) δ 9.50 (s, 1H), 8.75 (d, J = 8.0 Hz, 1H), 8.36 (s, 1H), 8.31 (d, J = 4.0 Hz, 1H), 8.24 (d, J = 8.0 Hz, 1H), 7.93~7.97 (m, 3H), 7.78 (s, 1H), 7.70~7.76 (m, 1H), 7.68 (s, 1H), 7.26~7.30 (m, 2H), 6.00 (d, J = 4.0 Hz, 1H), 3.17 (s, 3H), 3.02 (d, J = 4.0 Hz, 3H), 2.92 (s, 3H). | 587 |
| 263 | 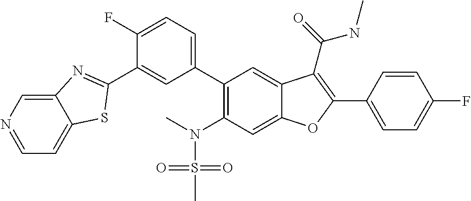 | ¹H-NMR (CDCl₃, 400 MHz) δ 9.45 (s, 1H), 8.55~8.59 (m, 2H), 7.94~7.99 (m, 4H), 7.71~7.75 (m, 2H), 7.67 (s, 1H), 7.23~7.27 (m, 2H), 5.89 (s, 1H), 3.21 (s, 3H), 3.03 (d, J = 4.8 Hz, 3H), 2.89 (s, 3H). | 605 |
| 264 | 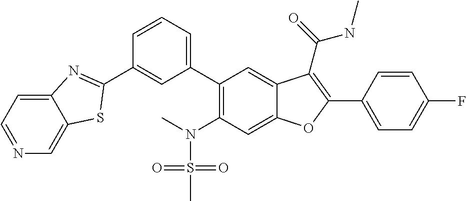 | ¹H-NMR (CDCl₃, 400 MHz) δ 9.47 (d, J = 5.6 Hz, 1H), 8.68 (d, J = 5.2 Hz, 1H), 8.32 (s, 1H), 8.18~8.21 (m, 2H), 7.89 (s, 1H), 7.84~7.89 (m, 2H), 7.72 (d, J = 8.0 Hz, 1H), 7.60~7.64 (m, 1H), 7.60 (s, 1H), 7.15~7.17 (m, 2H), 5.77 (br s, 1H), 3.09 (s, 3H), 2.92 (d, J = 4.0 Hz, 3H), 2.81 (s, 3H). | 587 |
| 265 | 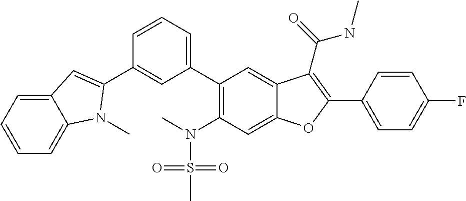 | ¹H-NMR (CDCl₃, 400 MHz) δ 7.96~7.99 (m, 2H), 7.88 (s, 1H), 7.62~7.68 (m, 3H), 7.57 (s, 2H), 7.50 (s, 1H), 7.40 (d, J = 8.0 Hz, 1H), 7.16~7.26 (m, 4H), 6.65 (s, 1H), 5.88 (s, 1H), 3.83 (s, 3H), 3.25 (s, 3H), 3.01 (d, J = 4.8 Hz, 3H), 2.67 (s, 3H). | 582 |
| 266 | 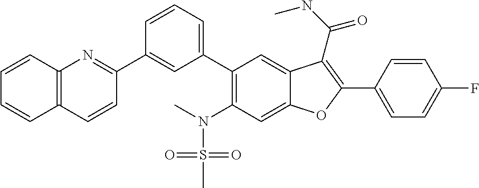 | ¹H-NMR (CDCl₃, 400 MHz) δ 8.79 (d, J = 8.0 Hz, 1H), 8.63 (d, J = 8.0 Hz, 1H), 8.35 (s, 1H), 8.24 (d, J = 8.4 Hz, 1H), 8.03~8.14 (m, 5H), 7.92 (s, 1H), 7.83~7.88 (m, 2H), 7.75 (t, J = 7.6 Hz, 1H), 7.62 (s, 1H), 7.23 (t, J = 8.4 Hz, 2H), 6.77 (s, 1H), 3.13 (s, 3H), 3.06 (d, J = 7.2 Hz, 3H), 2.93 (s, 3H). | 580 |
| 267 | 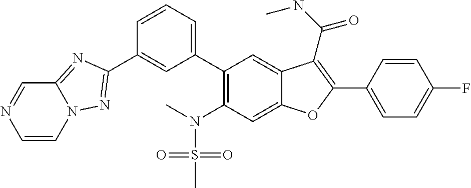 | ¹H-NMR (CDCl₃, 400 MHz) δ 9.24 (s, 1H), 8.50~8.51 (m, 1H), 8.33 (s, 1H), 8.27~8.29 (m, 1H), 8.14~8.15 (m, 1H), 7.88~7.92 (m, 2H), 7.82 (s, 1H), 7.55~7.60 (m, 3H), 7.56 (t, J = 8.4 Hz, 2H), 5.79~5.80 (m, 1H), 3.14 (s, 3H), 2.93 (d, J = 5.2 Hz, 3H), 2.58 (s, 3H). | 571 |
| 268 | 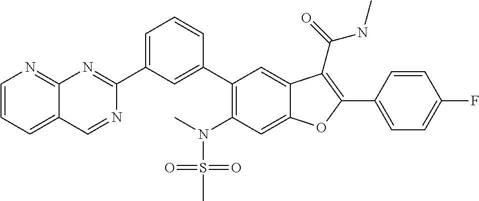 | ¹H-NMR (CDCl₃, 400 MHz) δ 9.56 (s, 1H), 9.28 (d, J = 2.4 Hz, 1H), 8.74~8.80 (m, 2H), 8.36~8.39 (m, 1H), 7.95~7.99 (m, 2H), 7.87 (s, 1H), 7.60~7.65 (m, 4H), 7.16~7.21 (m, 2H), 6.16 (br s, 1H), 3.21 (s, 3H), 3.00 (d, J = 4.8 Hz, 3H), 2.63 (s, 3H). | 582 |
| 269 | 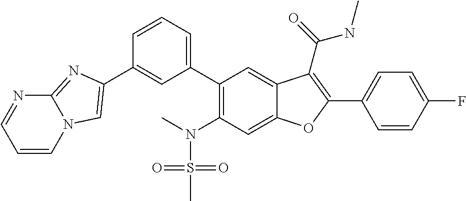 | ¹H-NMR (CDCl₃, 400 MHz) δ 8.68 (d, J = 3.2 Hz, 1H), 8.62 (d, J = 3.2 Hz, 1H), 8.03 (d, J = 9.6 Hz, 2H), 7.90~7.93 (m, 2H), 7.73~7.74 (d, J = 6.0 Hz, 1H), 7.35~7.46 (m, 3H), 7.09~7.13 (m, 3H), 6.79 (d, J = 4.4 Hz, 1H), 3.06 (s, 3H), 2.94 (d, J = 4.8 Hz, 3H), 2.80 (s, 3H). | 570 |
| 270 | 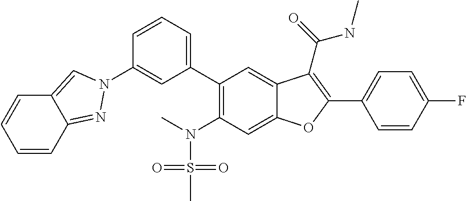 | ¹H-NMR (CDCl₃, 400 MHz) δ 8.55 (s, 1H), 8.08 (s, 1H), 7.94~7.98 (m, 3H), 7.90 (s, 1H), 7.70~7.80 (m, 2H), 7.61~7.68 (m, 2H), 7.49~7.51 (m, 1H), 7.32~7.38 (m, 1H), 7.20~7.26 (m, 2H), 7.08~7.14 (m, 1H), 5.85 (s, 1H), 3.24 (s, 3H), 2.30 (d, 3H), 2.80 (s, 3H). | 569 |
| 271 | 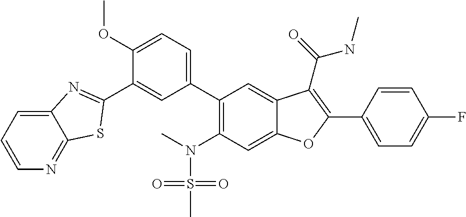 | ¹H-NMR (CDCl₃, 400 MHz) δ 8.66 (s, 2H), 8.37 (d, J = 7.6 Hz, 1H), 7.98~8.01 (m, 2H), 7.92 (s, 1H), 7.76 (t, J = 8.8 Hz, 1H), 7.67 (s, 1H), 7.54 (d, J = 5.6 Hz, 1H), 7.24 (t, J = 8.8 Hz, 3H), 5.99 (s, 1H), 4.19 (s, 3H), 3.19 (s, 3H), 3.03 (d, J = 5.2 Hz, 3H), 2.86 (s, 3H). | 617 |
| 272 | 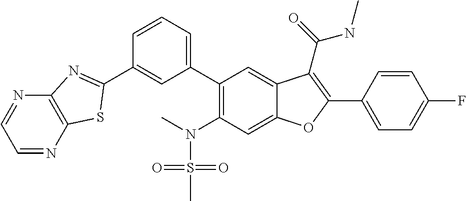 | ¹H-NMR (DMSO, 400 MHz) δ 8.82 (d, J = 2.4 Hz, 1H), 8.69 (d, J = 2.4 Hz, 1H), 8.56 (d, J = 4.4 Hz, 1H), 8.27 (s, 1H), 8.22~8.24 (m, 1H), 8.08 (s, 1H), 8.01~8.05 (m, 2H), 7.71~7.78 (m, 3H), 7.40~7.45 (m, 2H), 3.17 (s, 3H), 3.02 (s, 3H), 2.83 (d, J = 4.4 Hz, 3H). | 588 |
| 273 | 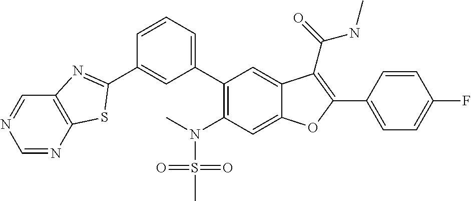 | ¹H-NMR (DMSO, 400 MHz) δ 9.27 (s, 1H), 9.05 (s, 1H), 8.17 (s, 1H), 8.08~8.09 (m, 1H), 7.85~7.89 (m, 3H), 7.63~7.65 (m, 1H), 7.57~7.59 (m, 2H), 7.14~7.18 (m, 2H), 5.78~5.79 (m, 1H), 3.11 (s, 3H), 2.93 (d, J = 8.0 Hz, 3H), 2.70 (s, 3H). | 588 |
| 274 | 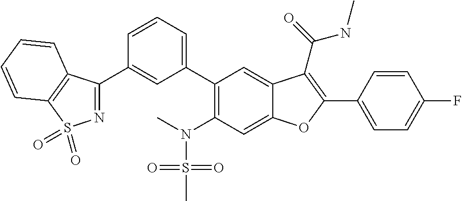 | ¹H-NMR (MeOD, 400 MHz) δ 7.94~7.98 (m, 4H), 7.86~7.89 (m, 2H), 7.79 (s, 1H), 7.67~7.72 (m, 2H), 7.63 (t, J = 8.0 Hz, 2H), 7.57 (s, 1H), 7.15 (t, J = 8.8 Hz, 2H), 5.79 (s, 1H), 3.16 (s, 3H), 2.92 (d, J = 4.8 Hz, 3H), 2.71 (s, 3H). | 618 |
| 275 | 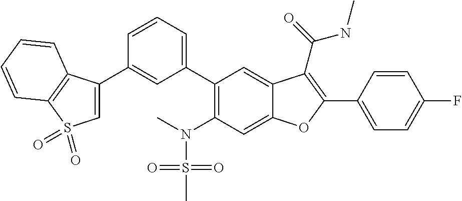 | ¹H NMR: (CDCl₃, 400 MHz) δ 7.85~7.87 (m, 2H), 7.78 (s, 1H), 7.72~7.74 (m, 1H), 7.46~7.57 (m, 8H), 7.12~7.16 (m, 2H), 6.70 (s, 1H), 5.77 (s, 1H), 3.12 (s, 3H), 2.92 (d, J = 4.8 Hz, 3H), 2.71 (s, 3H). | 617 |
| 276 | 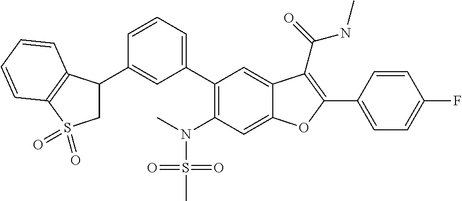 | ¹H NMR: (CDCl₃, 400 MHz) δ 7.84~7.88 (m, 2H), 7.71~7.74 (m, 2H), 7.28~7.53 (m, 6H), 7.22 (s, 1H), 7.12~7.16 (m, 3H), 5.76 (s, 1H), 4.80 (t, J = 8.0 Hz, 1H), 3.88~3.93 (m, 1H), 3.49~3.54 (m, 1H), 2.99 (s, 3H), 2.92 (d, J = 4.8 Hz, 3H), 2.60 (s, 3H). | 619 |
| 277 | 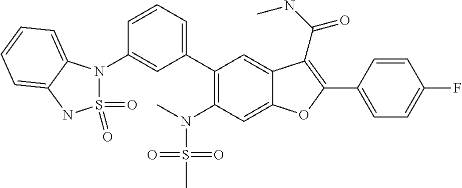 | ¹H-NMR (CDCl₃, 400 MHz) δ 7.87~7.90 (m, 2H), 7.77 (s, 1H), 7.54~7.59 (m, 5H), 7.12~7.17 (m, 2H), 6.93~6.97 (m, 4H), 6.64~6.66 (br s, 1H), 5.80~5.82 (m, 1H), 3.13 (s, 3H), 2.95 (d, J = 4.8 Hz, 3H), 2.58 (s, 3H). | 621 |
| 278 | 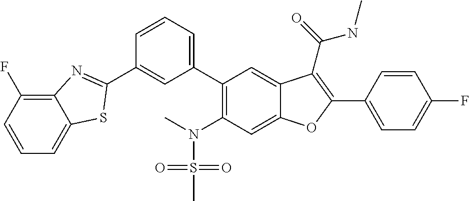 | ¹H-NMR (CDCl₃, 400 MHz) δ 8.21 (s, 1H), 8.12 (d, J = 7.2 Hz, 1H), 7.92~7.96 (m, 2H), 7.85 (s, 1H), 7.55~7.67 (m, 4H), 7.31~7.36 (m, 1H), 7.18 (t, J = 8.4 Hz, 3H), 5.92 (s, 1H), 3.14 (s, 3H), 2.98 (d, J = 4.8 Hz, 3H), 2.73 (s, 3H). | 604 |
| 279 | 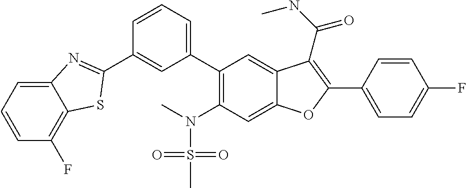 | ¹H-NMR (CDCl₃, 400 MHz) δ 8.18 (s, 1H), 8.08~8.11 (m, 1H), 7.90~7.94 (m, 2H), 7.84~7.85 (m, 2H), 7.55~7.64 (m, 3H), 7.42~7.48 (m, 1H), 7.09~7.20 (m, 3H), 6.06 (br s, 1H), 3.16 (s, 3H), 2.97 (d, J = 4.8 Hz, 3H), 2.62 (s, 3H). | 604 |
| 280 | 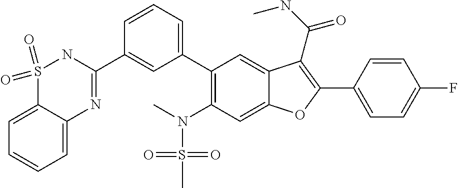 | ¹H-NMR (CDCl3, 400 MHz) δ 10.00 (s, 1H), 8.08 (d, J = 8.0 Hz, 2H), 7.85~8.05 (m, 4H), 7.49~7.59 (m, 3H), 7.33~7.42 (m, 1H), 7.16~7.19 (m, 2H), 7.12~7.14 (m, 2H), 5.93 (d, J = 4.0 Hz, 1H), 3.22 (s, 3H), 2.93 (d, J = 4.0 Hz, 3H), 2.70 (s, 3H). | 633 |
| 343 | 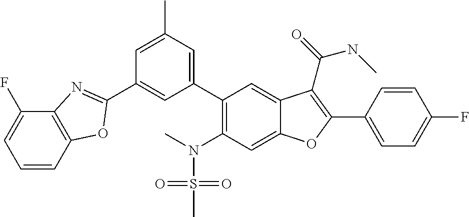 | ¹H-NMR (CDCl₃, 400 MHz) δ 8.18 (s, 2H), 7.97~8.18 (m, 2H), 7.90 (s, 1H), 7.68 (s, 1H), 7.54 (s, 1H), 7.44 (d, J = 7.6 Hz, 1H), 7.34~7.37 (m, 1H), 7.22~7.27 (m, 2H), 7.10~7.15 (m, 1H), 5.93~5.95 (br s, 1H), 3.21 (s, 3H), 3.04 (d, J = 4.8 Hz, 3H), 2.74 (s, 3H), 2.56 (s, 3H). | 602 |
| 404 | 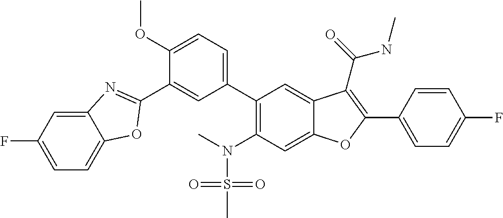 | ¹H-NMR (CDCl₃, 400 MHz) δ 8.13 (s, 1H), 7.85~7.88 (m, 2H), 7.79 (s, 1H), 7.62~7.66 (m, 1H), 7.55 (s, 1H), 7.45~7.48 (m, 2H), 7.05~7.15 (m, 3H), 7.02~7.05 (m, 1H), 5.85 (s, 1H), 4.00 (s, 3H), 3.09 (s, 3H), 2.94 (d, J = 4.8 Hz, 3H), 2.73 (s, 3H). | 618 |
| 405 | 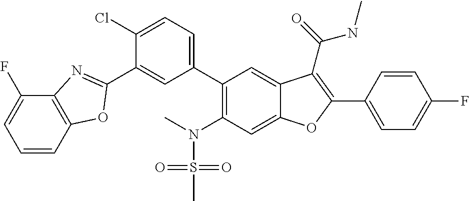 | ¹H-NMR (CDCl₃, 400 MHz) δ 8.26 (d, J = 1.2 Hz, 1H), 7.91~7.94 (m, 2H), 7.86 (s, 1H), 7.62~7.63 (m, 3H), 7.42~7.44 (m, 1H), 7.32~7.37 (m, 1H), 7.17~7.22 (m, 2H), 7.08~7.12 (m, 1H), 5.86 (s, 1H), 3.15 (s, 3H), 2.98 (d, J = 4.8 Hz, 3H), 2.83 (s, 3H). | 622 |
| 406 | 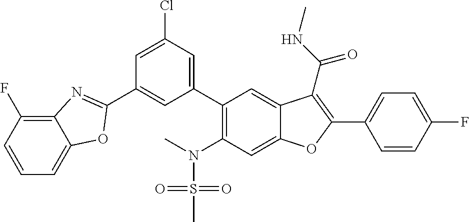 | ¹H-NMR (CDCl₃, 400 MHz) δ 8.15 (s, 1H), 8.10 (s, 1H), 7.86~7.89 (m, 2H), 7.82 (s, 1H), 7.60 (t, J = 2.8 Hz, 2H), 7.34~7.36 (m, 1H), 7.25~7.31 (m, 1H), 7.13~7.17 (m, 2H), 7.04 (t, J = 8.8 Hz, 1H), 5.86 (d, J = 4.4 Hz, 1H), 3.13 (s, 3H), 2.94 (d, J = 4.8 Hz, 3H), 2.74 (s, 3H). | 622 |
| 407 | 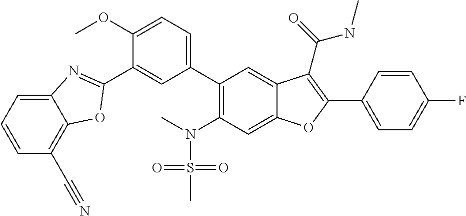 | ¹H-NMR (CDCl₃, 400 MHz) δ 8.18 (m, 1H), 8.16 (d, J = 4.8 Hz, 1H), 8.10 (s, 1H), 7.96~8.00 (m, 3H), 7.89~7.91 (m, 1H), 7.71~7.74 (m, 1H), 7.61 (s, 1H), 7.53~7.57 (m, 1H), 7.36~7.41 (m, 2H), 3.99 (s, 3H), 3.13 (s, 3H), 2.97 (s, 3H), 2.78 (d, J = 8.0 Hz, 3H). | 625 |
| 408 | 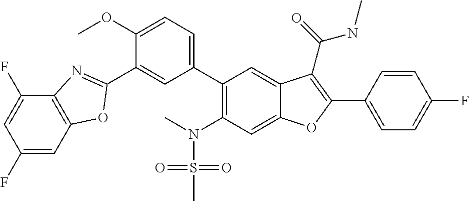 | ¹H-NMR (CDCl₃, 400 MHz) δ 8.17 (d, J = 2.0 Hz, 1H), 7.89~7.93 (m, 2H), 7.83 (s, 1H), 7.65~7.68 (m, 1H), 7.59 (s, 1H), 7.13~7.19 (m, 4H), 6.86~6.91 (m, 1H), 6.34 (d, J = 4.8 Hz, 1H), 4.06 (s, 3H), 3.15 (s, 3H), 3.01 (d, J = 4.8 Hz, 3H), 2.82 (s, 3H). | 636 |
| 409 | 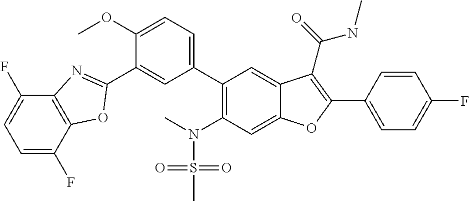 | ¹H-NMR (CDCl₃, 400 MHz) δ 8.26 (s, 1H), 7.95~7.98 (m, 2H), 7.88 (s, 1H), 7.74 (d, J = 8.4 Hz, 1H), 7.65 (s, 1H), 7.20~7.23 (m, 3H), 7.02~7.11 (m, 2H), 6.07 (s, 1H), 4.11 (s, 3H), 3.20 (s, 3H), 3.05 (d, J = 4.8 Hz, 3H), 2.57 (s, 3H). | 636 |
| 410 | 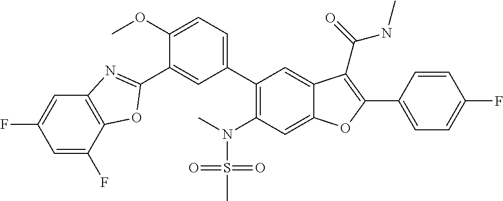 | ¹H-NMR (CDCl₃, 400 MHz) δ 8.19 (d, J = 2.0 Hz, 1H), 7.91~7.93 (m, 2H), 7.90 (s, 1H), 7.70~7.72 (m, 1H), 7.62 (s, 1H), 7.34 (d, J = 8.0 Hz, 1H), 7.18~7.22 (m, 3H), 6.89~6.94 (m, 1H), 6.01 (d, J = 4.0 Hz, 1H), 4.07 (s, 3H), 3.17 (s, 3H), 2.99 (d, J = 4.0 Hz, 3H), 2.80 (s, 3H). | 636 |

### Example 281

### 2-(4-fluorophenyl)-N-methyl-6-(N-methylmethylsulfonamido)-5-(3-(6-(methylsulfonamido)benzo[d]oxazol-2-yl)phenyl)benzofuran-3-carboxamide

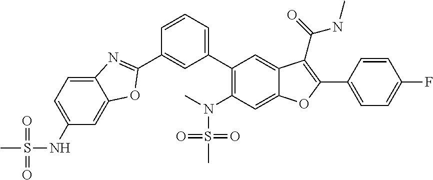

### Step 1: 5-(3-(6-aminobenzo[d]oxazol-2-yl)phenyl)-2-(4-fluorophenyl)-N-methyl-6-(N-methylmethylsulfonamido)benzofuran-3-carboxamide

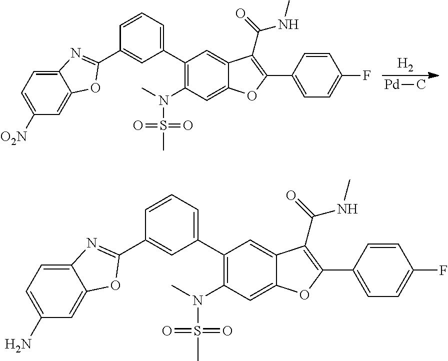

To a solution of the compound of Example 195 (530 mg, 0.13 mmol) in MeOH (10 mL), Pd/C (10 mg) was added, and the resulting reaction mixture was allowed to stir under 40 psi of H₂ atmosphere for 24 hours at 25° C. The reaction mixture was filtered, concentrated in vacuo and the residue obtained was purified using flash column chromatography (PE:EtOAc=2:1) to provide 5-(3-(6-aminobenzo[d]oxazol-2-yl)phenyl)-2-(4-fluorophenyl)-N-methyl-6-(N-methylmethylsulfonamido)benzofuran-3-carboxamide (420 mg, 85%). ¹H-NMR (DMSO, 400 MHz) δ 8.55 (s, 1H), 8.00˜8.11 (m, 5H), 7.59˜7.63 (m, 3H), 7.38˜7.40 (m, 3H), 6.80 (s, 1H), 6.62˜6.64 (d, J=8.4 Hz, 1H), 5.47 (s, 2H), 3.12 (s, 3H), 2.93 (s, 3H), 2.79˜2.80 (d, J=4.0 Hz, 3H). MS (M+H)⁺: 585.

### Step 2: 2-(4-fluorophenyl)-N-methyl-6-(N-methylmethylsulfonamido)-5-(3-(6-(methylsulfonamido)benzo[d]oxazol-2-yl)phenyl)benzofuran-3-carboxamide

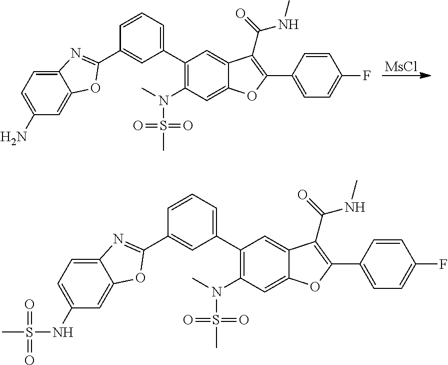

To a solution of 5-(3-(6-aminobenzo[d]oxazol-2-yl)phenyl)-2-(4-fluorophenyl)-N-methyl-6-(N-methylmethylsulfonamido)benzofuran-3-carboxamide (50 mg, 0.13 mmol) and pyridine (0.2 mL) in 1 mL of dry dichloromethane, MsCl (50 mg, 0.44 mmol) was added dropwise at 0° C. After stirred at room temperature for 4 hours, the mixture was quenched with 20% aq. NH₄Cl, then extracted with dichloromethane and washed with brine, dried over Na₂SO₄, filtered and concentrated in vacuo. The residue obtained was purified using preparative HPLC to provide 2-(4-fluorophenyl)-N-methyl-6-(N-methylmethylsulfonamido)-5-(3-(6-(methylsulfonamido)benzo[d]oxazol-2-yl)phenyl)benzofuran-3-carboxamide (43 mg, 90.1%).

¹H-NMR (CDCl₃, 400 MHz) δ 8.17˜8.23 (m, 3H), 7.88=7.92 (m, 2H), 7.80 (s, 1H), 7.55=7.60 (m, 4H), 7.25 (s, 1H), 7.12˜7.14 (m, 2H), 7.06˜7.08 (m, 1H), 5.79 (s, 1H), 3.13 (s, 3H), 2.93˜2.94 (d, J=4.8 Hz, 3H), 2.60 (s, 3H), 2.56 (s, 3H). MS (M+H)⁺: 663.

The following compounds of the present invention were prepared using the method described in Example 281 and substituting the appropriate reactants and/or reagents.

| | | | MS |
|---|---|---|---|
| Compound | Structure | NMR | (M + H)⁺ |
| 282 | 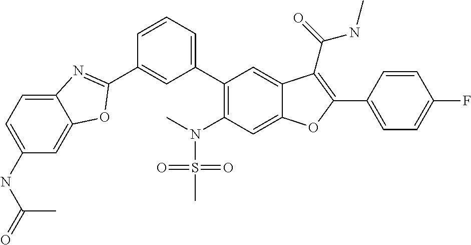 | ¹H-NMR (CDCl₃, 400 MHz) δ 8.17~8.23 (m, 3H), 7.88~7.92 (m, 2H), 7.80 (s, 1H), 7.55~7.60 (m, 4H), 7.25 (s, 1H), 7.12~7.14 (m, 2H), 7.06~7.08 (m, 1H), 5.79 (s, 1H), 3.13 (s, 3H), 2.94 (d, J = 4.8 Hz, 3H), 2.60 (s, 3H), 2.16 (s, 3H). | 628 |
| 283 | 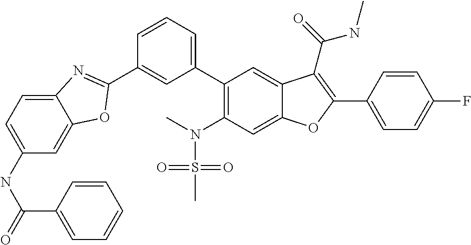 | ¹H-NMR (CDCl₃, 400 MHz) δ 10.61 (s, 1H), 8.60~8.61 (m, 1H), 8.48 (s, 1H), 8.33 (s, 1H), 8.27~ 8.29 (m, 1H), 8.04~8.12 (m, 5H), 7.46~7.89 (m, 10H), 3.23 (s, 3H), 3.04 (s, 3H), 2.89 (d, J = 4.4 Hz, 3H). | 690 |
| 284 | 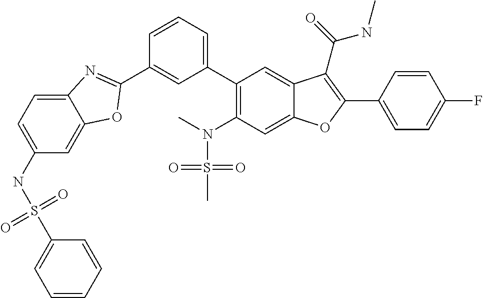 | ¹H-NMR (CDCl₃, 400 MHz) δ 8.15 (m, 2H), 7.86~7.90 (m, 2H), 7.81 (s, 1H), 7.67~7.70 (m, 2H), 7.58~7.61 (m, 2H), 7.49~7.56 (m, 2H), 7.42~7.47 (m, 2H), 7.35~7.39 (m, 2H), 7.13~7.15 (m, 2H), 6.81~6.86 (m, 1H), 6.54 (s, 1H), 5.81 (d, J = 4.8 Hz, 1H) 3.13 (s, 3H), 2.94 (d, J = 4.8 Hz, 3H), 2.62 (s, 3H). | 726 |
| 285 | 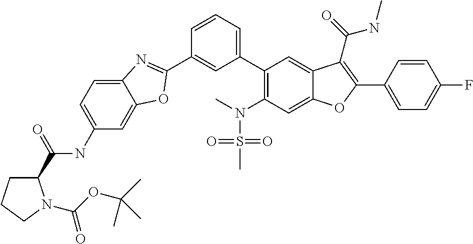 | ¹H-NMR (CDCl₃, 400 MHz) δ 9.87 (br s, 1H), 8.17~8.24 (m, 3H), 7.92~7.96 (m, 2H), 7.83 (s, 1H), 7.57~7.63 (m, 4H), 7.14~7.21 (m, 3H), 6.08 (br s, 1H), 4.52 (br s, 1H), 3.37~3.47 (m, 2H), 3.18 (s, 3H), 2.98 (d, J = 4.8 Hz, 3H), 2.65 (s, 3H), 2.53 (br s, 1H), 1.95 (br s, 3H), 1.52 (s, 9H). | 783 |
| 286 | 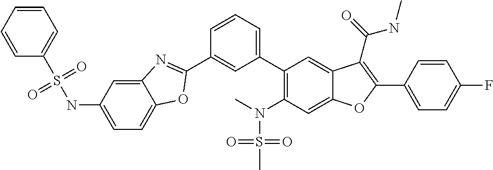 | ¹H-NMR (CDCl₃, 400 MHz) δ 8.25 (s, 1H), 8.15 (d, J = 8.0 Hz, 1H), 7.87~7.90 (m, 2H), 7.82 (s, 1H), 7.62~7.68 (m, 2H), 7.52~7.60 (m, 3H), 7.45~7.48 (m, 1H), 7.34~7.40 (m, 4H), 7.15 (t, J = 8.8 Hz, 2H), 7.04~7.07 (m, 1H), 6.44 (s, 1H), 5.77 (d, J = 3.6 Hz, 1H), 3.12 (s, 3H), 2.92 (d, J = 4.8 Hz, 3H), 2.63 (s, 3H). | 726 |
| 287 | 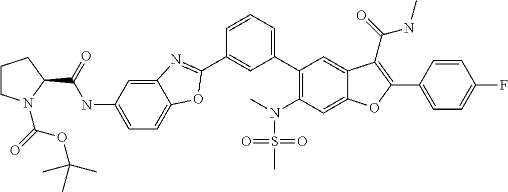 | ¹H-NMR (CDCl₃, 400 MHz) δ 8.25 (s, 1H), 8.20 (d, J = 8.0 Hz, 1H), 7.91 (t, J = 8.0 Hz, 3H), 7.81 (s, 1H), 7.59 (t, J = 9.6 Hz, 2H), 7.54 (d, J = 7.6 Hz, 1H), 7.45 (s, 2H), 7.15 (t, J = 8.4 Hz, 2H), 5.79 (d, J = 4.4 Hz, 1H), 4.44~4.48 (m, 1H), 3.27~3.41 (m, 2H), 3.12 (s, 3H), 2.94 (d, J = 5.2 Hz, 3H), 2.62 (s, 3H), 2.50~2.57 (m, 1H), 1.86~1.90 (m, 3H), 1.45 (s, 9H). | 783 |

### Example 288

### 5-(3-(6-cyanobenzo[d]thiazol-2-yl)phenyl)-2-(4-fluorophenyl)-N-methyl-6-(N-methylmethylsulfonamido)benzofuran-3-carboxamide

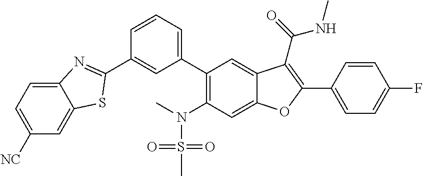

### Step 1: 2-(4-fluorophenyl)-5-(3-formylphenyl)-N-methyl-6-(N-methylmethylsulfonamido)benzofuran-3-carboxamide (Q1)

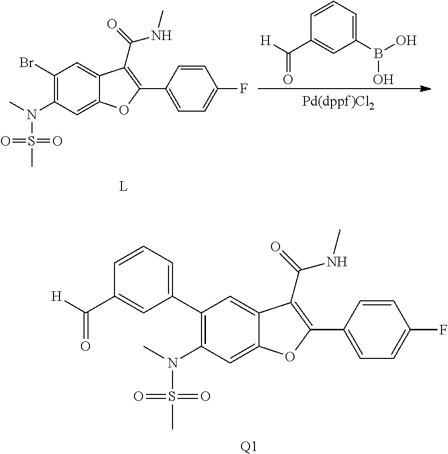

To a degassed solution of 3-formylphenylboronic acid (440 mg, 2.64 mmol) in dry DMF (20 mL) was added Compound L (1.0 g, 2.20 mmol), K₃PO₄ (1.2 g, 4.40 mmol) and Pd(dppf)Cl₂(20 mg). Then the reaction mixture was placed under N₂ atmosphere and stirred at 100° C. for 6 hours. After cooled to room temperature and filtered, the filtrate was washed with H₂O, brine, and dried over Na₂SO₄, filtered and concentrated in vacuo. The residue obtained was purified using column chromatography (PE:EtOAc=3:1) to provide aryl aldehyde Q1 (760 mg, 72.1%) as white solid. ¹H-NMR (CDCl₃, 400 MHz) δ 10.05 (s, 1H), 7.98˜7.88 (m, 4H), 7.82 (s, 1H), 7.75 (s, 1H), 7.62˜7.59 (m, 2H), 7.59˜7.16 (m, 2H), 5.96 (s, 1H), 3.10 (s, 3H), 2.96 (s, 3H), 2.69 (s, 3H). MS (M+H)⁺: 481.5.

### Step 2: 5-(3-(6-cyanobenzo[d]thiazol-2-yl)phenyl)-2-(4-fluorophenyl)-N-methyl-6-(N-methylmethylsulfonamido)benzofuran-3-carboxamide

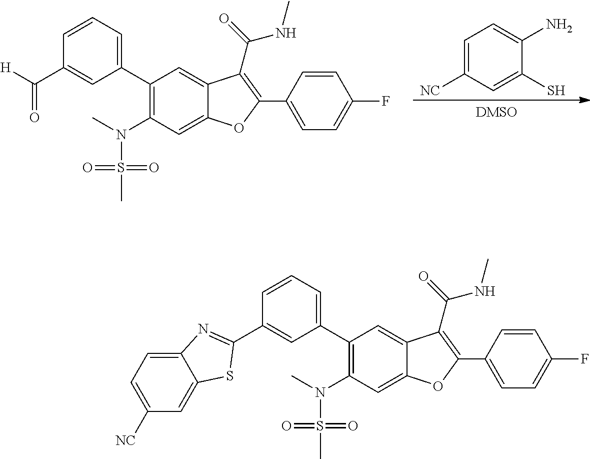

A mixture of the aryl aldehyhyde Q1 (150 mg, 0.31 mmol) and 4-amino-3-mercaptobenzonitrile (56 mg, 0.37 mmol) in DMSO (3 mL) was allowed to stir at 200° C. for 2 hours. After cooled, the mixture was diluted with water and extracted with EtOAc. The combined organic phases were washed with brine, dried over Na₂SO₄, filtered and concentrated in vacuo. The residue was purified using preparative HPLC to provide the target compound (150 mg, 79%). ¹H-NMR (CDCl₃, 400 MHz) δ 8.27˜8.28 (m, 2H), 8.14˜8.19 (m, 2H), 7.94˜7.99 (m, 3H), 7.76˜7.84 (m, 1H), 7.63˜7.72 (m, 3H), 7.23˜7.25 (m, 2H), 5.91˜5.92 (m, 1H), 3.19 (s, 3H), 3.20 (d, J=4.4 Hz, 3H), 2.81 (s, 3H). MS (M+H)⁺: 611.

The following compounds of the present invention were prepared using the method described in Example 288 and substituting the appropriate reactants and/or reagents.

| | | | MS |
|---|---|---|---|
| Compound | Structure | NMR | (M + H)⁺ |
| 289 | 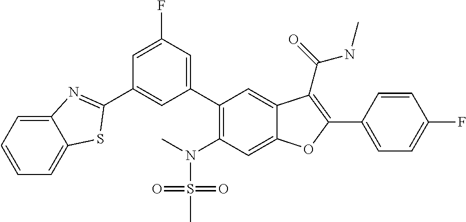 | ¹H-NMR (CDCl₃, 400 MHz) δ 8.01~8.10 (m, 1H), 7.89~7.98 (m, 6H), 7.68 (s, 1H), 7.53~7.57 (m, 1H), 7.43~7.48 (m, 1H), 7.34~7.37 (m, 1H), 7.24 (t, J = 8.8 Hz, 2H), 5.97 (br s, 1H), 3.21 (s, 3H), 3.04 (d, J = 4.8 Hz, 3H), 2.84 (s, 3H). | 604 |
| 290 | 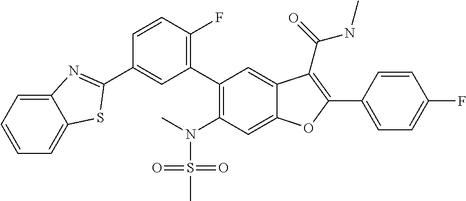 | ¹H-NMR (CDCl₃, 400 MHz) δ 8.13~8.16 (m, 2H), 8.02~8.05 (m, 1H), 7.90~7.96 (m, 2H), 7.88 (d, J = 4.0 Hz, 2H), 7.67 (s, 1H), 7.47~7.51 (m, 1H), 7.37~7.41 (m, 1H), 7.26~7.31 (m, 1H), 7.17~7.12 (m, 2H), 6.00 (br s, 1H), 3.25 (s, 3H), 2.99 (d, J = 4.8 Hz, 3H), 2.64 (s, 3H). | 604 |
| 291 | 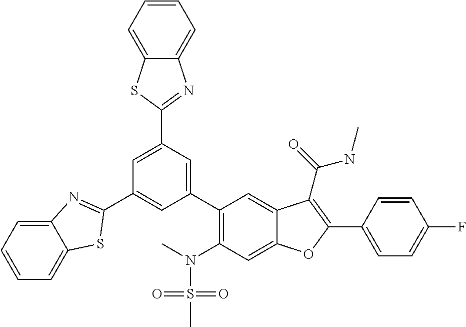 | ¹H-NMR (CDCl₃, 400 MHz) δ 8.78 (s, 2H), 8.29 (s, 2H), 8.05 (d, J = 8.0 Hz, 2H), 7.90~7.95 (m, 4H), 7.86 (s, 1H), 7.65 (s, 1H), 7.47~7.51 (m, 2H), 7.38~7.41 (m, 2H), 7.13~7.18 (m, 2H), 3.17 (s, 3H), 2.94 (s, 3H), 2.82 (s, 3H). | 719 |
| 292 | 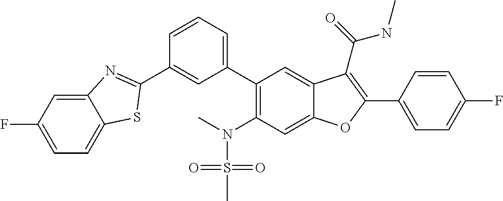 | ¹H-NMR (CDCl₃, 400 MHz) δ 8.15 (s, 1H), 8.04~8.06 (m, 1H), 7.87~7.91 (m, 2H), 7.82 (s, 1H), 7.76~7.79 (m, 1H), 7.66~7.69 (m, 1H), 7.57 (d, J = 9.2 Hz, 1H), 7.51~7.56 (m, 2H), 7.09~7.17 (m, 3H), 5.80 (d, J = 3.6 Hz, 1H), 3.11 (s, 3H), 2.92 (d, J = 5.2 Hz, 3H), 2.66 (s, 3H). | 604 |
| 293 | 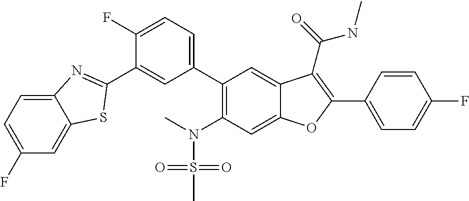 | ¹H-NMR (CDCl₃, 400 MHz) δ 8.39~8.41 (m, 1H), 7.94~7.97 (m, 1H), 7.87~7.90 (m, 2H), 7.82 (s, 1H), 7.56~7.58 (m, 3H), 7.25~7.30 (m, 1H), 7.13~7.17 (m, 3H), 5.78 (s, 1H), 3.09 (s, 3H), 2.93 (d, J = 4.8 Hz, 3H), 2.77 (s, 3H). | 622 |
| 294 | 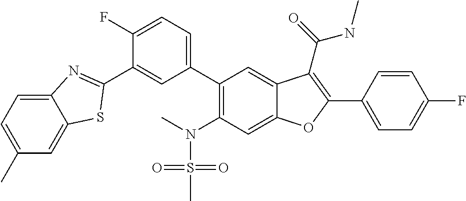 | ¹H-NMR (CDCl₃, 400 MHz) δ 8.41 (d, J = 5.6 Hz, 1H), 7.88~7.92 (m, 3H), 7.81 (s, 1H), 7.68 (s, 1H), 7.55~7.58 (m, 2H), 7.25~7.29 (m, 2H), 7.12~7.15 (m, 2H), 5.81 (br s, 1H), 3.08 (s, 3H), 2.94 (d, J = 4.8 Hz, 3H), 2.76 (s, 3H), 2.46 (s, 3H). | 618 |
| 295 | 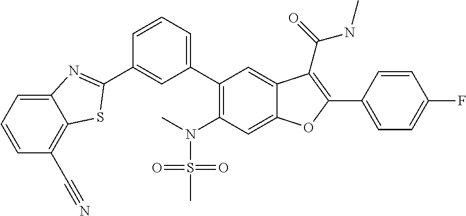 | ¹H-NMR (CDCl₃, 400 MHz) δ 8.22~8.24 (m, 1H), 8.12 (s, 1H), 8.03~8.05 (m, 1H), 7.81~7.85 (m, 2H), 7.78 (s, 1H), 7.68~7.70 (m, 1H), 7.52~7.62 (m, 4H), 7.13~ 7.18 (m, 2H), 6.12~6.13 (m, 1H), 3.12 (s, 3H), 2.96 (d, J = 5.2 Hz, 3H), 2.68 (s, 3H). | 611 |
| 296 | 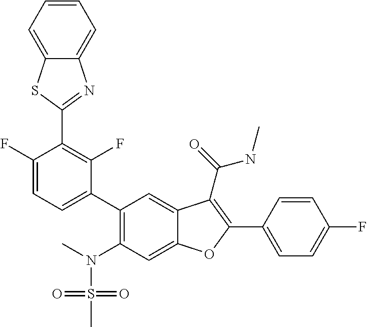 | ¹H-NMR (CDCl₃, 400 MHz) δ 8.18 (d, J = 4.0 Hz, 1H), 7.91~7.99 (m, 3H), 7.85 (s, 1H), 7.64 (s, 1H), 7.53~7.58 (m, 2H), 7.45~7.50 (m, 1H), 7.17~7.23 (m, 3H), 5.93 (br s, 1H), 3.23 (s, 3H), 3.00 (d, J = 4.8 Hz, 3H), 2.75 (s, 3H). | 622 |

### Example 297

### 5-(3-(6-(aminomethyl)benzo[d]thiazol-2-yl)phenyl)-2-(4-fluorophenyl)-N-methyl-6-(N-methylmethylsulfonamido)benzofuran-3-carboxamide

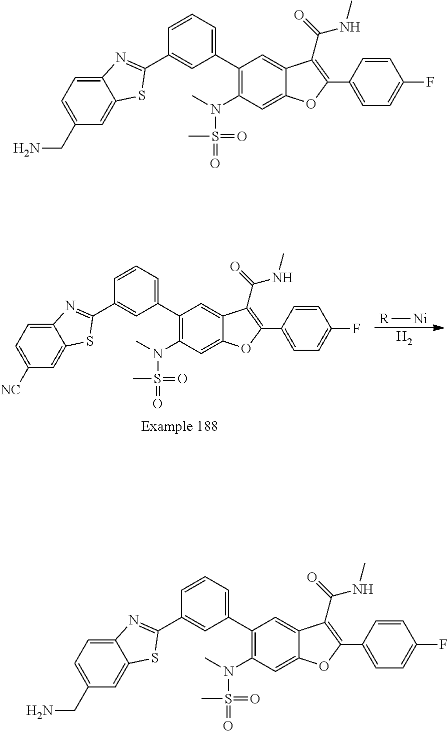

To a solution of the compound of Example 188 (120 mg, 0.20 mmol) and NH₄OH (0.5 mL) in MeOH (10 mL), was added Raney-Ni (100 mg). The resulting solution was degassed and then was shaken under hydrogen gas atmosphere (30 psi) for about 15 hours. The reaction mixture was filtered and the collected solid was washed with MeOH. The filtrate and washing were combined and concentrated in vacuo to provide the target compound (80 mg, 66%). ¹H-NMR (MeOD, 400 MHz) δ 8.23 (s, 1H), 8.12˜8.14 (m, 1H), 8.06˜8.09 (m, 2H), 7.94˜7.97 (m, 2H), 7.82 (s, 1H), 7.74 (s, 1H), 7.69 (s, 1H), 7.57˜7.67 (m, 2H), 7.22˜7.26 (m, 2H), 4.24 (s, 2H), 3.18 (s, 3H), 2.92 (s, 3H), 2.89 (s, 3H). MS (M+H)⁺: 615.

### Example 298

### 5-(3-(6-((dimethylamino)methyl)benzo[d]thiazol-2-yl)phenyl)-2-(4-fluorophenyl)-N-methyl-6-(N-methylmethylsulfonamido)benzofuran-3-carboxamide

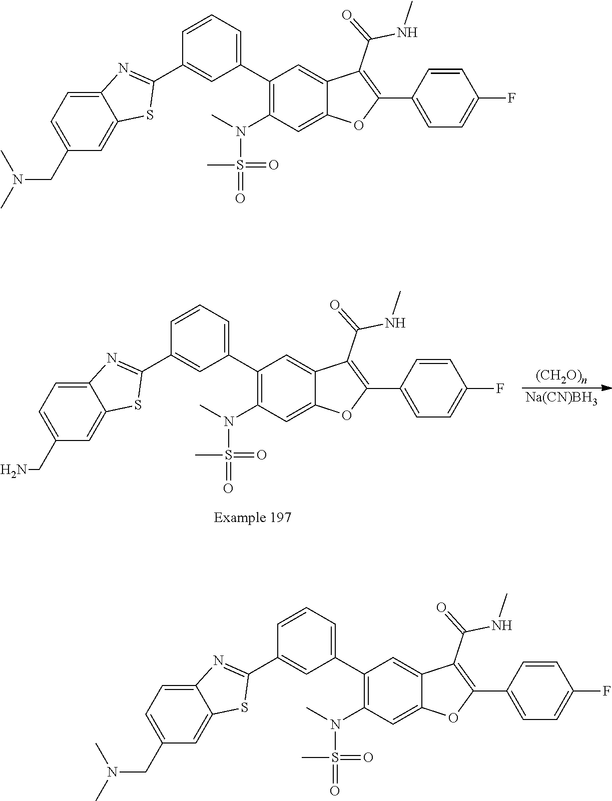

CF₃COOH (0.1 mL) was added to a solution of the compound of Example 197 (50 mg, 0.08 mmol) and paraformaldehyde (5 mg, 0.16 mmol) in MeOH (2 mL). The resulting reaction was allowed to stir at room temperature for 3 hours, then Na(CN)BH₃ (10 mg, 0.16 mmol) was added. The reaction mixture was allowed to stir at room temperature for about 15 hours, then was quenched with saturated NH₄Cl solution and extracted with EtOAc. The combined organic phases were washed with brine, dried over Na₂SO₄, filtered and concentrated in vacuo. The residue obtained was purified using preparative HPLC to provide the target compound (20 mg, 38%). ¹H-NMR (CDCl₃, 400 MHz) δ 8.14 (s, 1H), 8.03˜8.08 (m, 2H), 7.99 (s, 1H), 7.87˜7.91 (m, 2H), 7.83 (s, 1H), 7.53˜7.60 (m, 3H), 7.44˜7.46 (m, 1H), 7.13˜7.17 (m, 2H), 5.82˜5.83 (m, 1H), 4.25 (s, 2H), 3.11 (s, 3H), 2.92 (d, J=8.0 Hz, 3H), 2.75 (s, 6H), 2.67 (s, 3H). MS (M+H)⁺: 643.

### Example 299

### 5-(3-(3H-imidazo[4,5-b]pyridin-2-yl)phenyl)-2-(4-fluorophenyl)-N-methyl-6-(N-methylmethylsulfonamido)benzofuran-3-carboxamide

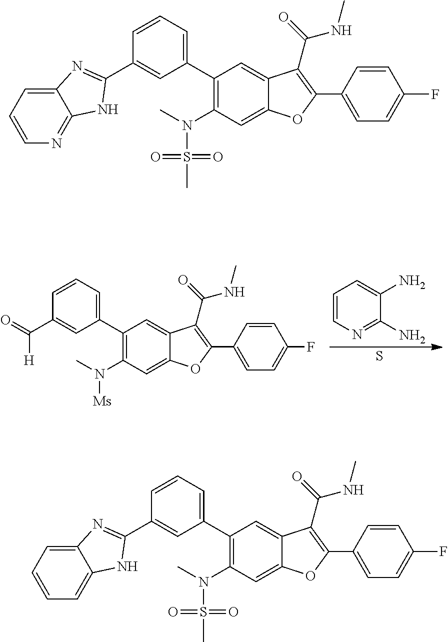

A solution of compound Q1 (100 mg, 0.385 mmol) in pyridine-2,3-diamine (58 mg, 0.42 mmol) was heated to 160° C. and allowed to stir at this temperature for 2 hours. The reaction mixture was cooled to room temperature, quenched with water, and extracted with EtOAc. The organic layer was concentrated in vacuo and the resulting residue was purified using prep-TLC (DCM:MeOH=20:1) to provide the target compound (50 mg, 53.7%). ¹H-NMR (CDCl₃, 400 MHz) δ 8.26˜8.29 (m, 2H), 8.07 (s, 1H), 7.74˜7.82 (m, 4H), 7.41˜7.52 (m, 3H), 7.25˜7.27 (m, 1H), 7.05˜7.15 (m, 3H), 3.14 (s, 3H), 2.94 (s, 3H), 2.82 (d, J=4.8 Hz, 3H).

MS (M+H)⁺: 570.

The following compounds of the present invention were prepared using the method described in Example 299 and substituting the appropriate reactants and/or reagents.

| | | | MS |
|---|---|---|---|
| Compound | Structure | NMR | (M + H)⁺ |
| 300 | 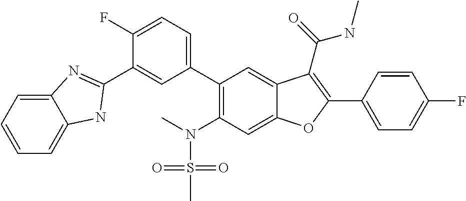 | ¹H-NMR (CDCl₃, 400 MHz) δ 8.53~8.56 (m, 1H), 7.98~8.01 (m, 2H), 7.88 (s, 1H), 7.63~7.70 (m, 3H), 7.61 (s, 1H), 7.32~7.34 (m, 4H), 7.20~7.25 (m, 2H), 6.14 (s, 1H), 3.15 (s, 3H), 3.04 (d, J = 4.8 Hz, 3H), 2.92 (s, 3H). | 587 |
| 301 | 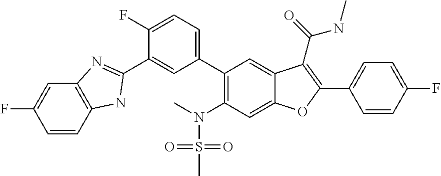 | ¹H-NMR (CDCl₃, 400 MHz) δ 8.15~8.17 (m, 1H), 7.76~7.79 (m, 2H), 7.70 (s, 1H), 7.64 (m, 3H), 7.43 (s, 1H), 7.33~7.36 (m, 1H), 7.14~7.17 (m, 1H), 7.03~7.07 (m, 2H), 6.95~7.00 (m, 1H), 3.00~3.01 (m, 6H), 2.92 (s, 3H). | 605 |
| 302 | 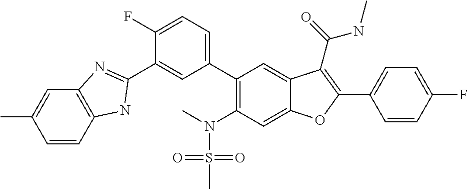 | ¹H-NMR (CDCl₃, 400 MHz) δ 8.24~8.26 (m, 1H), 7.81~7.87 (m, 3H), 7.73 (s, 1H), 7.63~7.64 (m, 1H), 7.49~7.51 (m, 1H), 7.39~7.41 (m, 2H), 7.16 (s, 1H), 7.03~7.07 (m, 2H), 6.88~6.93 (m, 1H), 3.02 (s, 3H), 2.99 (d, J = 4.0 Hz, 3H), 2.91 (s, 3H), 2.37 (s, 3H). | 601 |
| 303 | 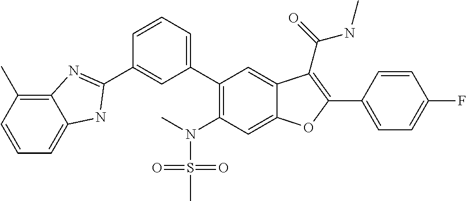 | ¹H-NMR (CDCl₃, 400 MHz) δ 8.33 (s, 1H), 8.04 (d, J = 8.0 Hz, 1H), 7.88~7.92 (m, 2H), 7.78 (s, 1H), 7.51~7.55 (m, 3H), 7.44 (d, J = 7.6 Hz, 1H), 7.17~7.27 (m, 2H), 7.10~7.13 (m, 3H), 3.03 (s, 3H), 2.94 (s, 6H), 2.57 (s, 3H). | 583 |
| 304 | 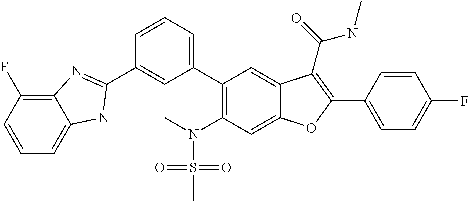 | ¹H-NMR (CDCl₃, 400 MHz) δ 8.19 (d, J = 7.2 Hz, 1H), 8.13 (s, 1H), 7.92~7.96 (m, 2H), 7.82 (s, 1H), 7.54~7.61 (m, 3H), 7.36 (s, 1H), 7.14~7.19 (m, 3H), 6.95 (t, J = 8.0 Hz, 1H), 3.36 (s, 3H), 2.97 (s, 3H), 2.78 (s, 3H). | 587 |
| 305 | 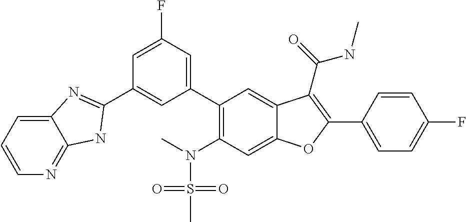 | ¹H-NMR (MeOD, 400 MHz) δ 8.48~8.51 (d, J = 4.4 Hz, 1H), 8.30~8.32 (m, 1H), 8.15 (s, 1H), 7.95~7.99 (m, 3H), 7.91 (s, 1H), 7.78 (s, 1H), 7.52~7.56 (m, 2H), 7.25~7.30 (t, J = 8.8 Hz, 2H), 3.24 (s, 3H), 2.93~2.95 (m, 6H). | 588 |
| 306 | 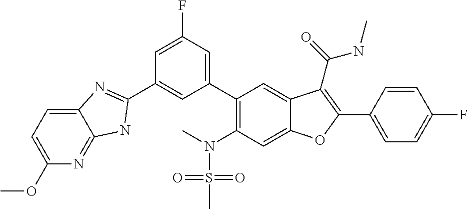 | ¹H-NMR (MeOD, 400 MHz) δ 8.52 (d, J = 4.8 Hz, 1H), 8.04~8.06 (m, 1H), 7.97~8.00 (m, 3H), 7.68 (s, 1H), 7.38~7.42 (m, 3H), 7.19 (s, 1H), 7.07 (s, 1H), 3.89 (s, 3H), 3.17 (s, 3H), 2.97 (s, 3H), 2.79~2.80 (m, 3H). | 618 |
| 307 | 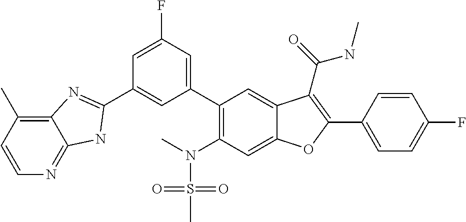 | ¹H-NMR (MeOD, 400 MHz) δ 8.39 (d, J = 4.8 Hz, 1H), 8.23 (s, 1H), 8.04~8.07 (m, 1H), 7.95~7.98 (m, 2H), 7.92 (s, 1H), 7.79 (s, 1H), 7.54~7.56 (m, 1H), 7.47~7.48 (m, 1H), 7.25~7.30 (t, J = 8.8 Hz, 2H), 3.24 (s, 3H), 2.98 (s. 3H), 2.92 (s, 3H), 2.3 (m, 3H). | 602 |
| 308 | 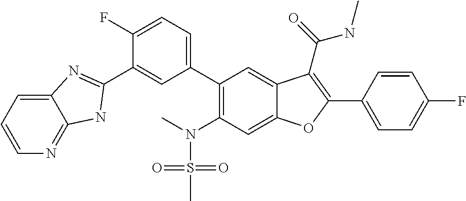 | ¹H-NMR: (CDCl₃, 400 MHz) δ 8.54 (s, 1H), 8.41 (s, 1H), 8.08~9.00 (m, 1H), 7.89~7.92 (m, 2H), 7.86 (s, 1H), 7.64~7.65 (m, 1H), 7.24~7.30 (m, 3H), 7.14 (t, J = 8.4 Hz, 3H), 6.17 (s, 1H), 3.08 (s, 3H), 2.98 (d, J = 4.8 Hz, 3H), 2.84 (s, 3H). | 588 |
| 309 | 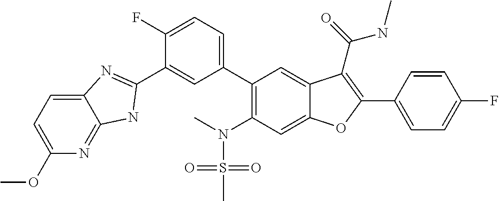 | ¹H-NMR (MeOD, 400 MHz) δ 8.12 (s, 1H), 8.06 (d, J = 9.2 Hz, 1H), 7.91~7.97 (m, 3H), 7.82~7.86 (m, 1H), 7.76 (s, 1H), 7.50~7.55 (m, 1H), 7.27 (t, J = 8.8 Hz, 2H), 7.00 (d, J = 9.2 Hz, 1H), 4.03 (s, 3H), 3.23 (s, 3H), 2.97 (s, 3H), 2.93 (s, 3H). | 618 |
| 310 | 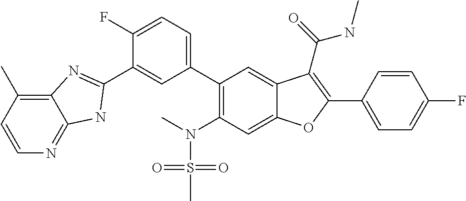 | ¹H-NMR (MeOD, 400 MHz) δ 8.34 (d, J = 2.4 Hz, 1H), 8.32 (d, J = 2.4 Hz, 1H), 7.95~7.98 (m, 2H), 7.90 (s, 1H), 7.79~7.83 (m, 1H), 7.76 (s, 1H), 7.47~7.56 (m, 2H), 7.27 (t, J = 8.8 Hz, 2H), 3.21 (s, 3H), 2.97 (s, 3H), 2.93 (s, 3H), 2.88 (s, 3H). | 602 |
| 311 | 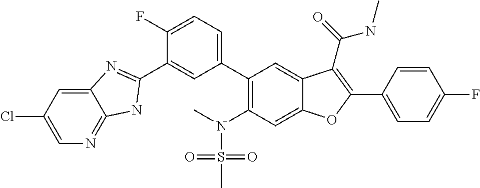 | ¹H-NMR (DMSO, 400 MHz) δ 9.32 (s, 1H), 9.05 (s, 1H), 8.20~8.21 (m, 1H), 7.70~7.78 (m, 4H), 7.46~7.48 (m, 2H), 7.29~7.31 (m, 3H), 3.10 (s, 3H), 3.01 (s, 3H), 2.85 (s, 3H). | 622 |
| 312 | 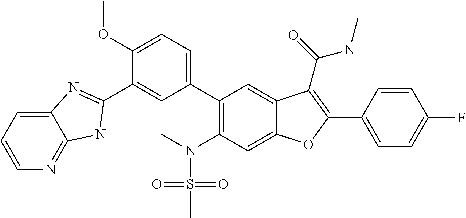 | ¹H-NMR: (CDCl₃, 400 MHz) δ 8.48 (s, 1H), 8.30~8.35 (m, 2H), 7.90~7.94 (m, 2H), 7.82 (s, 1H), 7.69~7.71 (m, 1H), 7.50 (s, 1H), 7.43~7.45 (m, 1H), 7.08~7.14 (m, 3H), 6.62 (br s, 1H), 4.12 (s, 3H), 3.08 (s, 3H), 2.94 (d, J = 4.8 Hz, 3H), 2.80 (s, 3H). | 600 |
| 313 | 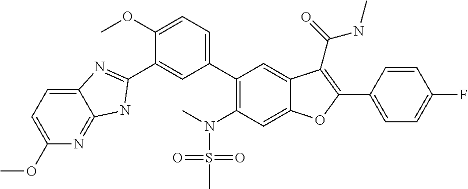 | ¹H-NMR (CDCl₃, 400 MHz) δ 9.52~9.61 (m, 1H), 8.36 (s, 1H), 7.96~8.00 (m, 1H), 7.88~7.92 (m, 2H), 7.78~7.81 (m, 3H), 7.44 (s, 1H), 7.05~7.10 (m, 3H), 6.82~6.83 (m, 1H), 4.07 (s, 3H), 3.92 (s, 3H), 3.01 (s, 3H), 2.96 (d, J = 4.8 Hz, 3H), 2.86 (s, 3H). | 630 |
| 314 | 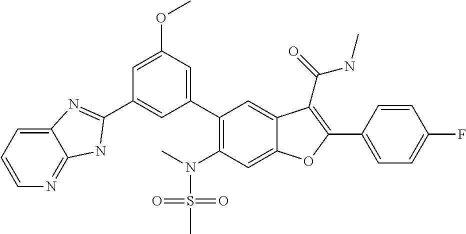 | ¹H-NMR (MeOD, 400 MHz) δ 8.54 (d, J = 4.4 Hz, 1H), 8.43 (d, J = 4.0 Hz, 1H), 7.95~7.98 (m, 2H), 7.93 (s, 1H), 7.89 (s, 1H), 7.83 (s, 1H), 7.76 (s, 1H), 7.64~7.67 (m, 1H), 7.30 (s, 1H), 7.28 (t, J = 8.8 Hz, 2H), 3.97 (s, 3H), 3.18 (s, 3H), 2.98 (s, 3H), 2.92 (s, 3H). | 600 |
| 315 | 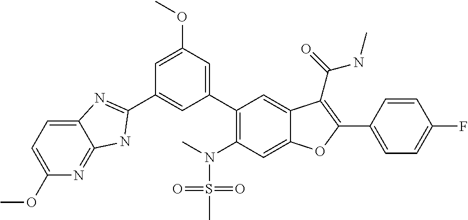 | ¹H-NMR (MeOD, 400 MHz) δ 7.85~7.91 (m, 2H), 7.77 (s, 1H), 7.65 (s, 1H), 7.64 (s, 1H), 7.59 (s, 1H), 7.58 (s, 1H), 7.32 (s, 1H), 7.17 (t, J = 8.8 Hz, 2H), 6.86 (d, J = 8.8 Hz, 1H), 3.94 (s, 3H), 3.89 (s, 3H), 3.11 (s, 3H), 2.91 (s, 3H), 2.86 (s, 3H). | 630 |
| 316 | 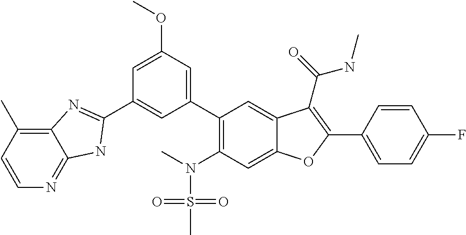 | ¹H-NMR (CDCl₃, 400 MHz) δ 8.32 (d, J = 8.0 Hz, 1H), 7.86~7.92 (m, 3H), 7.80~7.82 (m, 2H), 7.69 (s, 1H), 7.43 (d, J = 4.0 Hz, 1H), 7.351~7.35 (m, 1H), 7.17~7.22 (m, 2H), 3.89 (s, 3H), 3.08 (s, 3H), 2.91 (s, 3H), 2.84 (s, 3H), 2.78 (s, 3H). | 614 |
| 317 | 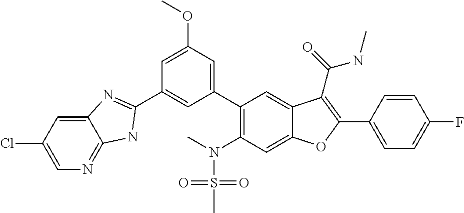 | ¹H-NMR (CDCl₃, 400 MHz) δ 8.22 (s, 1H), 7.84~7.89 (m, 3H), 7.73 (s, 1H), 7.66~7.67 (m, 1H), 7.52 (s, 1H), 7.09~7.14 (m, 4H), 3.87 (s, 3H), 3.04 (s, 3H), 2.87 (s, 3H), 2.77 (s, 3H). | 634 |
| 318 | 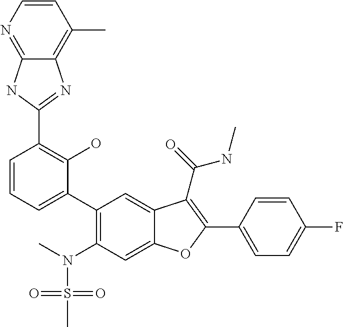 | ¹H-NMR (CDCl₃, 400 MHz) δ 8.18~8.19 (m, 1H), 7.97~8.01 (m, 3H), 7.77 (s, 1H), 7.66 (s, 1H), 7.45~7.47 (m, 1H), 7.11~7.21 (m, 4H), 3.39 (s, 3H), 3.26 (s, 3H), 2.96 (s, 3H), 2.66 (s, 3H). | 600 |
| 319 | 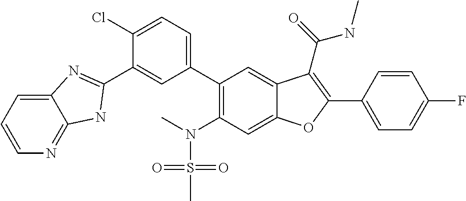 | ¹H-NMR (CDCl₃, 400 MHz) δ 8.51 (d, J = 4.0 Hz, 1H), δ 8.26 (d, J = 8.0 Hz, 1H), 8.05 (s, 1H), 7.98~8.00 (m, 2H), 7.97 (s, 1H), 7.90 (s, 1H), 7.74~7.78 (m, 2H), 7.49~7.52 (m, 2H), 7.26~7.30 (m, 2H), 3.22 (s, 3H), 2.99 (s, 3H), 2.96 (s, 3H). | 604 |
| 320 | 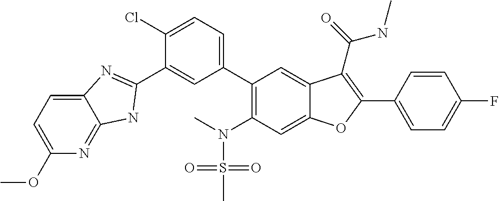 | ¹H-NMR (400 MHz, MeOH) δ 7.96~8.02 (m, 4H), 7.90 (s, 1H), 7.74~7.77 (m, 3H), 7.25~7.30 (m, 2H), 6.90 (d, J = 8.0 Hz, 1H), 4.02 (s, 3H), 3.22 (s, 3H), 2.98 (s, 3H), 2.96 (s, 3H). | 634 |
| 321 | 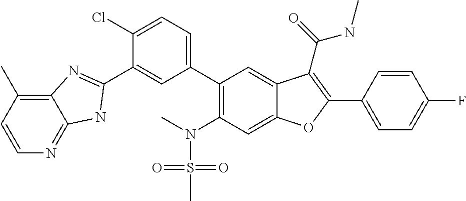 | ¹H-NMR (MeOD, 400 MHz) δ 8.45 (d, J = 8.0 Hz, 1H), 7.96~7.99 (m, 3H), 7.91 (s, 1H), 7.76~7.87 (d, J = 8.0 Hz, 3H), 7.50~751 (d, J = 4.0 Hz, 1H), 7.36~7.31 (m, 2H), 3.21 (s, 3H), 3.00 (s, 3H), 2.95 (s, 3H), 2.84 (s, 3H). | 618 |
| 322 | 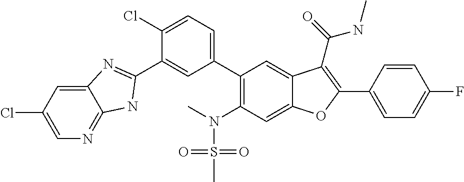 | ¹H-NMR (MeOD, 400 MHz) δ 8.42~8.43 (d, J = 4.0 Hz, 1H), 8.11~8.12 (d, J = 4.0 Hz, 1H), 7.97~8.01 (m, 3H), 7.89 (s, 1H), 7.79 (s, 1H), 7.24 (s, 2H), 7.26~7.30 (m, 2H), 3.22 (s, 3H), 2.97 (s, 3H), 2.96 (s, 3H) | 638 |
| 323 | 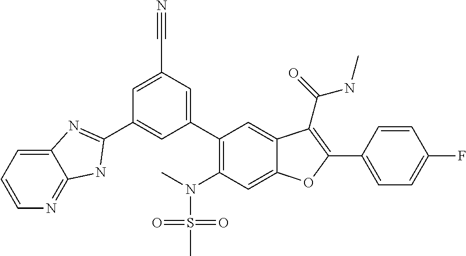 | ¹H-NMR (MeOD, 400 MHz) δ 8.50 (s, 1H), 8.42~8.45 (m, 2H), 8.23~8.25 (m, 1H), 7.98 (s, 1H), 7.79~7.91 (m, 3H), 7.73 (s, 1H), 7.45~7.48 (m, 1H), 7.17~7.21 (m, 2H), 3.21 (s, 3H), 2.86~2.89 (m, 6H). | 595 |
| 324 | 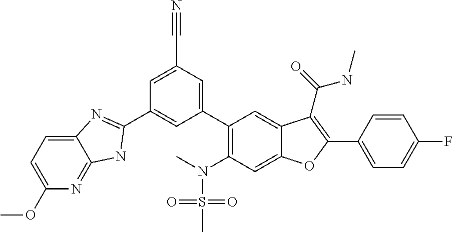 | ¹H-NMR (MeOD, 400 MHz) δ 8.42~8.44 (m, 2H), 8.06 (s, 1H), 7.98~8.02 (m, 4H), 7.83 (s, 1H), 7.28~7.32 (m, 2H), 6.87~6.89 (m, 1H), 4.02 (s, 3H), 3.31 (s, 3H), 2.97~2.99 (m, 6H). | 625 |
| 325 | 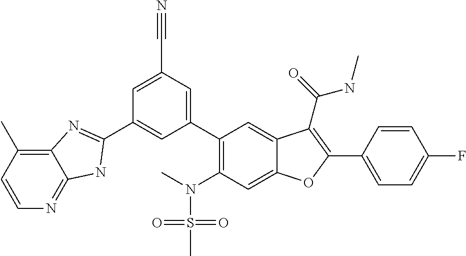 | ¹H-NMR (MeOD, 400 MHz) δ 8.72 (s, 1H), 8.68 (s, 1H), 8.49~8.51 (m, 1H), 8.15 (s, 1H), 8.98~9.00 (m, 3H), 7.85 (s, 1H), 7.61~.63 (m, 1H), 7.29~7.34 (m, 2H), 3.31 (s, 3H), 3.01 (s, 3H), 2.92~2.95 (m, 6H). | 609 |
| 326 | 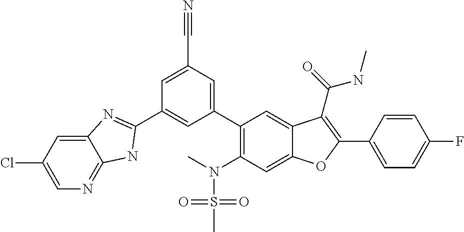 | ¹H-NMR (MeOD, 400 MHz) δ 8.51 (s, 1H), 8.47 (s, 1H), 8.38 (s, 1H), 8.07 (s, 1H), 8.03 (s, 1H), 7.94~7.97 (m, 3H), 7.99 (s, 1H), 7.24~7.28 (m, 2H), 3.29 (s, 3H), 2.94~2.95 (m, 6H). | 629 |
| 327 | 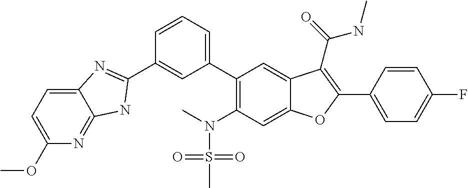 | ¹H-NMR (CDCl₃, 400 MHz) δ 8.13 (s, 1H), 7.80~7.94 (m, 4H), 7.65 (s, 1H), 7.48 (d, J = 7.6 Hz, 1H), 7.07~7.35 (m, 4H), 6.77 (d, J = 8.0 Hz, 1H), 3.86 (s, 3H), 2.96 (s, 3H), 2.92 (d, J = 4.4 Hz, 3H), 2.88 (s, 3H). | 600 |
| 328 | 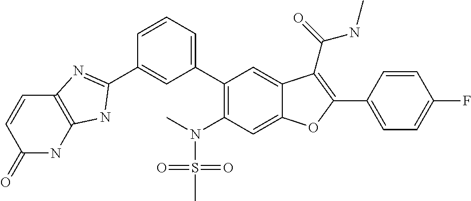 | ¹H NMR (CDCl₃, 400 MHz) δ 8.03~8.07 (m, 2H), 7.78~7.81 (m, 2H), 7.38~7.45 (m, 3H), 7.04~7.15 (m, 3H), 6.79 (s, 1H), 6.27 (d, J = 2.0 Hz, 1H), 3.42 (s, 3H), 2.91 (d, J = 4.4 Hz, 3H), 2.83 (s, 3H). | 586 |
| 329 | 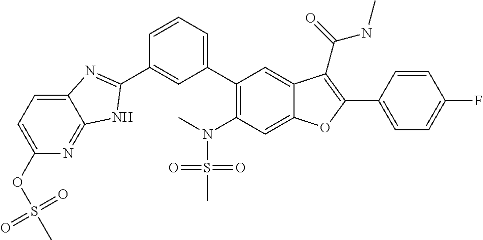 | ¹H NMR (CDCl₃, 400 MHz) δ 8.19 (s, 1H), 8.15 (d, J = 3.6 Hz, 1H), 8.09 (d, J = 7.6 Hz, 1H), 7.86~7.89 (m, 3H), 7.57 (s, 2H), 7.43 (s, 1H), 7.06~7.18 (m, 3H), 6.05 (s, 1H), 3.49 (s, 3H), 3.12 (s, 3H), 2.94 (d, J = 4.8 Hz, 3H), 2.83 (s, 3H). | 664 |
| 330 | 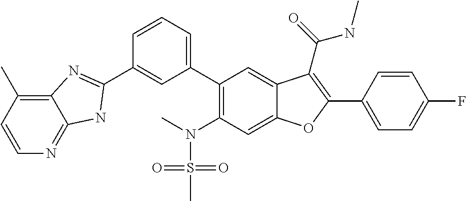 | ¹H-NMR (CDCl₃, 400 MHz) δ 7.96~8.00 (m, 2H), 7.84 (s, 1H), 7.81 (s, 1H), 7.76 (d, J = 7.6 Hz, 1H), 7.67~7.71 (m, 1H), 7.54~7.61 (m, 4H), 7.13~7.18 (m, 2H), 6.72 (d, J = 4 Hz, 1H), 4.08 (s, 3H), 3.14 (s, 3H), 2.96 (d, J = 4.4 Hz, 3H), 2.88 (s, 3H). | 584 |
| 331 | 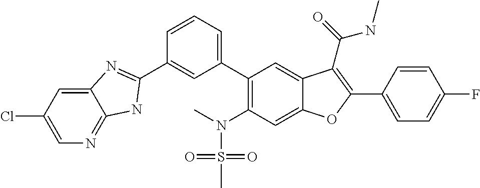 | ¹H-NMR (CDCl₃, 400 MHz) δ 8.54 (d, J = 4.0 Hz, 1H), 8.35 (s, 1H), 8.27 (s, 1H), 8.23 (s, 1H), 8.17 (s, 1H), 8.04 (s, 1H), 7.97~8.01 (m, 2H), 7.63~7.66 (m, 2H), 7.38~7.43 (m, 2H), 3.14 (s, 3H), 2.92 (s, 3H), 2.80 (d, J = 4.0 Hz, 3H). | 604 |
| 332 | 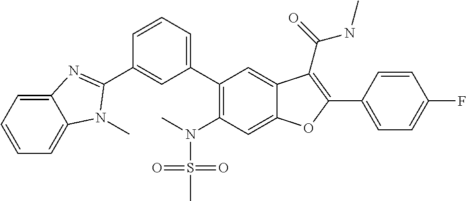 | ¹H-NMR (CDCl₃, 400 MHz) δ 7.96~8.00 (m, 4H), 7.84 (s, 1H), 7.81 (s, 1H), 7.76 (d, J = 7.6 Hz, 1H), 7.67~7.71 (m, 1H), 7.54~7.61 (m, 4H), 7.13~7.18 (m, 2H), 6.72 (d, J = 4 Hz, 1H), 4.08 (s, 3H), 3.14 (s, 3H), 2.96 (d, J = 4.4 Hz, 3H), 2.88 (s, 3H). | 583 |
| 333 | 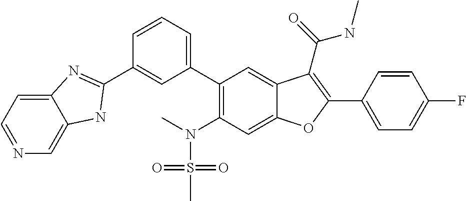 | ¹H-NMR (MeOD, 400 MHz) δ 9.23 (s, 1H), 8.53 (d, J = 6.4 Hz, 1H), 8.39 (s, 1H), 8.28 (d, J = 8 Hz, 1H), 8.09 (d, J = 6.4 Hz, 1H), 7.79~8.01 (m, 2H), 7.92 (s, 1H), 7.71~7.83 (m, 3H), 7.27~7.33 (m, 2H), 3.24 (s, 3H), 2.95 (s, 3H), 2.92 (s, 3H). | 570 |
| 334 | 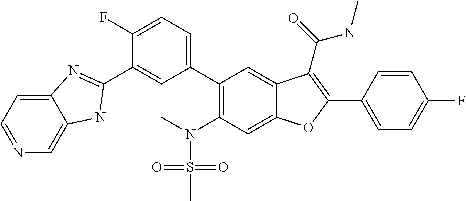 | ¹H-NMR (CDCl₃, 400 MHz) δ 9.78 (s, 1H), 8.67~8.70 (m, 1H), 8.61~8.63 (m, 1H), 8.39~8.41 (m, 1H), 8.24~8.30 (m, 1H), 8.16 (s, 1H), 7.85~7.88 (m, 2H), 7.75 (s, 1H), 7.50 (t, J = 8.8 Hz, 2H), 5.70 (s, 1H), 3.28 (s, 3H), 3.09 (s, 3H), 2.88 (d, J = 4.8 Hz, 3H). | 588 |
| 335 | 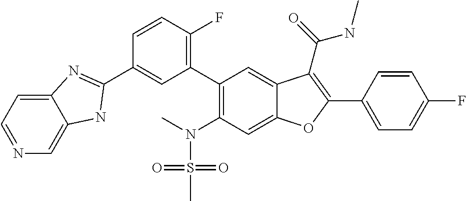 | ¹H-NMR (CDCl₃, 400 MHz) 9.08 (s, 1H), 8.27~8.32 (m, 2H), 8.12 (s, 1H), 7.86~7.89 (m, 2H), 7.77 (s, 2H), 7.51 (s, 1H), 7.27 (t, J = 8.8 Hz, 1H), 7.11 (t, J = 8.0 Hz, 2H), 6.53 (s, 1H), 3.23 (s, 3H), 2.90 (d, J = 4.0 Hz, 3H), 2.70 (s, 3H). | 588 |
| 336 | 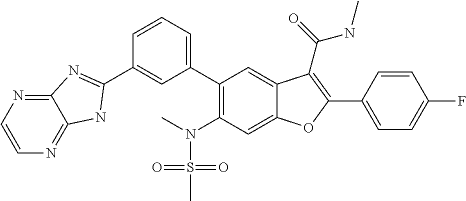 | ¹H-NMR (CDCl₃, 400 MHz) δ 8.30~8.57 (m, 4H), 7.99~8.06 (m, 3H), 7.69~7.70 (m, 3H), 7.40~7.44 (m, 2H), 3.18 (s, 3H), 2.94 (s, 3H), 2.81 (s, 3H). | 571 |
| 337 | 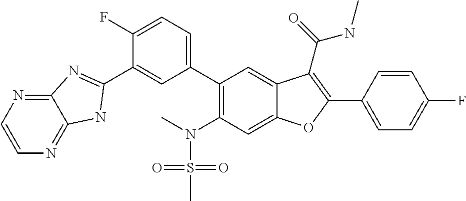 | ¹H-NMR (MeOD, 400 MHz,) δ 8.43 (s, 2H), 8.34 (d, J = 7.2 Hz, 1H), 7.96~8.00 (m, 2H), 7.88 (s, 1H), 7.76~7.79 (m, 2H), 7.43~7.47 (m, 1H), 7.24~7.28 (m, 2H), 3.22 (s, 3H), 2.94 (s, 3H), 2.93 (s, 3H). | 589 |
| 338 | 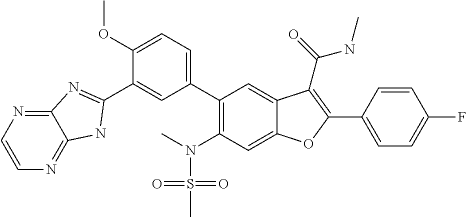 | ¹H-NMR (CDCl₃, 400 MHz) δ 10.78 (br s, 1H), 8.54 (s, 1H), 8.40 (s, 2H), 7.84~7.88 (m, 2H), 7.72~7.74 (m, 2H), 7.50 (s, 1H), 7.08~7.13 (m, 3H), 6.68 (s, 1H), 4.14 (s, 3H), 3.07 (s, 1H), 3.13 (s, 3H), 2.96 (d, J = 4.8 Hz, 3H), 2.82 (s, 3H). | 601 |
| 339 | 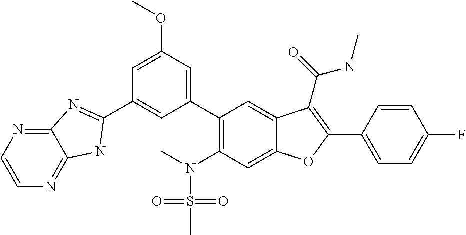 | ¹H-NMR (MeOD, 400 MHz) δ 8.29 (s, 2H), 7.87~7.90 (m, 2H), 7.79 (s, 1H), 7.75 (s, 1H), 7.72 (s, 1H), 7.67 (s, 1H), 7.28 (s, 1H), 7.15~7.20 (m, 2H), 3.87 (s, 3H), 3.09 (s, 3H), 2.86 (d, J = 4.8 Hz, 6H). | 601 |
| 340 | 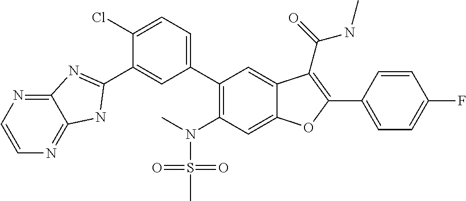 | ¹H-NMR (MeOD, 400 MHz) δ 8.49 (s, 2H), 7.97~8.01 (m, 3H), 7.90 (s, 1H), 7.74~7.78 (m, 3H), 7.26~7.30 (m, 2H), 3.22 (s, 3H), 2.98 (s, 3H), 2.96 (s, 3H). | 605 |
| 341 | 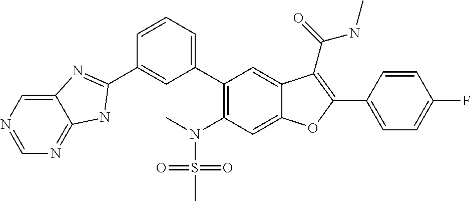 | ¹H-NMR (DMSO, 400 MHz) δ 9.32 (s, 1H), 9.05 (s, 1H), 8.20~8.21 (m, 1H), 7.70~7.77 (m, 4H), 7.46~7.48 (m, 2H), 7.29~7.31 (m, 3H), 3.10 (s, 3H), 3.01 (s, 3H), 2.85 (s, 3H). | 571 |
| 342 | 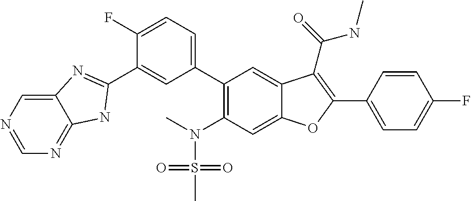 | ¹H-NMR (MeOD, 400 MHz) δ 9.04 (s, 1H), 8.89 (s, 1H), 8.28~8.3.1 (m, 1H), 7.87~7.91 (m, 2H), 7.81 (s, 1H), 7.67~7.73 (m, 2H), 7.36~7.41 (m, 2H), 7.36~7.41 (m, 1H), 7.19 (t, J = 8.8 Hz, 2H), 3.15 (s, 3H), 2.86 (s, 6H). | 589 |
| 343 | 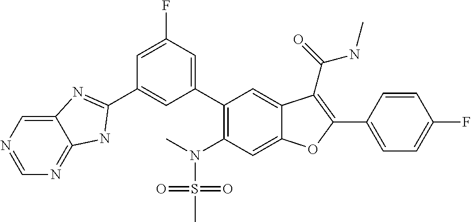 | ¹H-NMR (DMSO, 400 MHz) δ 9.10 (s, 1H), 8.95 (s, 1H), 8.52~8.53 (m, 1H), 8.15 (s, 1H), 7.97~8.08 (m, 4H), 7.70 (s, 1H), 7.50~7.52 (m, 1H), 7.38~7.42 (m, 2H), 3.18 (s, 3H), 2.98 (s. 3H), 2.79~2.80 (m, 3H). | 589 |
| 344 | 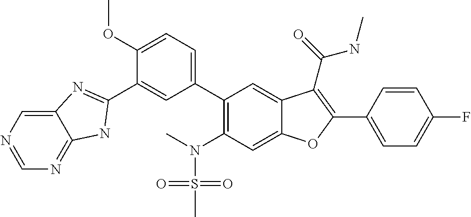 | ¹H-NMR (CDCl₃, 400 MHz) δ 9.02 (s, 1H), 8.91 (s, 1H), 8.51 (s, 1H), 7.90~7.94 (m, 2H), 7.77 (s, 1H), 7.68~7.70 (m, 1H), 7.55 (s, 1H), 7.12~7.18 (m, 3H), 4.17 (s, 3H), 3.33 (s, 1H), 3.13 (s, 3H), 2.93 (s, 3H), 2.80 (s, 3H). | 601 |
| 345 | 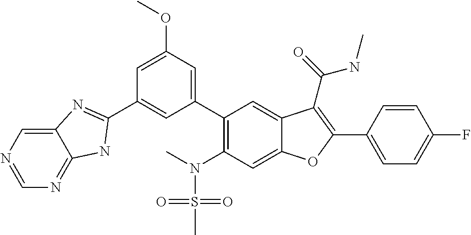 | ¹H-NMR (MeOD, 400 MHz) δ 9.10 (s, 1H), 8.98 (s, 1H), 7.95~7.98 (m, 2H), 7.85~7.89 (m, 2H), 7.81 (s, 1H), 7.74 (s, 1H), 7.39 (s, 1H), 7.24~7.28 (m, 2H), 3.97 (s, 3H), 317 (s, 3H), 2.95 (s, 3H), 2.92 (s, 3H). | 601 |
| 346 | 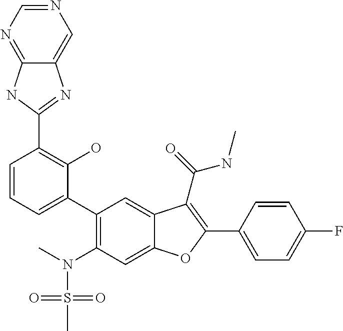 | ¹H-NMR (DMSO, 400 MHz) δ 9.14 (s, 1H), 8.95 (s, 1H), 8.48~8.49 (m, 1H), 8.16~8.18 (m, 1H), 7.95~7.99 (m, 3H), 7.52 (s, 1H), 7.37~7.41 (m, 3H), 7.11~7.15 (m, 1H), 3.16 (s, 3H), 2.79 (s, 3H), 2.77 (s, 3H). | 587 |
| 347 | 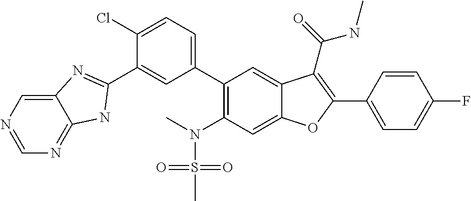 | ¹H-NMR (DMSO, 400 MHz) δ 9.29 (s, 1H), 9.06 (s, 1H), 8.59 (d, J = 4.0 Hz, 1H), 8.04~8.08 (m, 3H), 8.01 (d, J = 4.0 Hz, 1H), 7.83~7.85 (m, 2H), 7.76~7.78 (m, 2H), 3.21 (s, 3H), 3.10 (s, 3H), 2.88 (d, J = 8.0 Hz, 3H). | 605 |
| 348 | 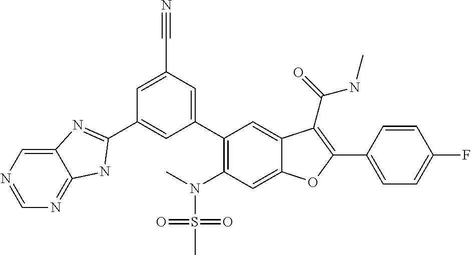 | ¹H-NMR (MeOD, 400 MHz) δ 9.21 (s, 1H), 9.05 (s, 1H), 8.61 (s, 1H) 8.56 (s, 1H), 8.10 (s, 1H), 7.97~7.99 (m, 3H), 7.78 (s, 1H), 7.27~7.31 (m, 2H), 3.31 (s, 3H), 2.97~3.00 (m, 6H). | 596 |
| 349 | 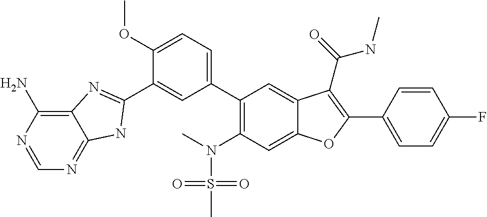 | ¹H-NMR (DMSO, 400 MHz) δ 8.55~8.56 (m, 1H), 8.30~8.31 (m, 1H), 8.04 (s, 1H), 8.04~7.97 (m, 2H), 7.68~7.70 (m, 1H), 7.59 (s, 1H), 7.40~7.45 (m, 3H), 4.11 (br s, 2H), 3.15 (d, J = 4.0 Hz, 6H), 3.00 (s, 3H), 2.80 (d, J = 4.8 Hz, 3H). | 616 |
| 350 | 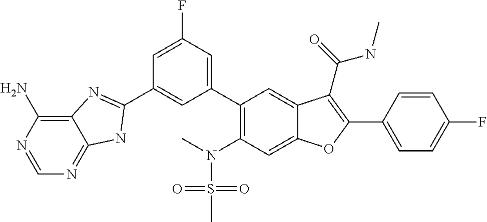 | ¹H-NMR (DMSO, 400 MHz) δ 8.07~8.26 (m, 5H), 7.78~7.81 (m, 1H), 7.42~7.50 (m, 3H), 7.33 (s, 1H), 4.20 (br s, 2H), 3.05 (s, 3H), 2.97 (s, 3H), 2.88 (s, 3H). | 604 |

### Example 351

### 5-(3-(benzo[b]thiophen-2-yl)phenyl)-2-(4-fluorophenyl)-N-methyl-6-(N-methylmethylsulfonamido)benzofuran-3-carboxamide

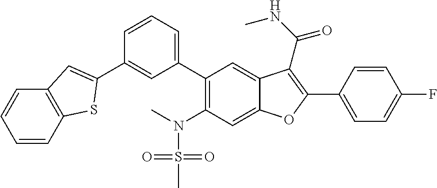

### Step 1: 2-(4-fluorophenyl)-N-methyl-6-(N-methylmethylsulfonamido)-5-(3-nitrophenyl)benzofuran-3-carboxamide

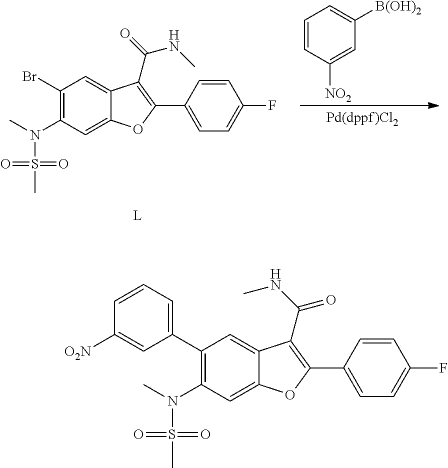

To a degassed solution of Compound L (prepared as described in Example 1, Step 11, 2.0 g, 4.39 mmol) and 3-nitrophenylboronic acid (880 mg, 5.27 mmol) in dry DMF (1.5 mL) were added Pd(dppf)Cl₂ (20 mg) and K₃PO₄ (1.86 g, 8.79 mmol) under N₂. The mixture was allowed to stir at 90° C. for about 15 hours. After the mixture was cooled to room temperature, diluted with EtOAc and filtered, the filtrate was washed with H₂O, brine, and dried over Na₂SO₄. After concentrated, the crude was purified using column chromatography (PE:EtOAc=3:1) to provide 2-(4-fluorophenyl)-N-methyl-6-(N-methylmethylsulfonamido)-5-(3-nitrophenyl)benzofuran-3-carboxamide (1.78 g, 84%). ¹H-NMR (CDCl₃, 400 MHz) δ 8.24 (s, 1H), 8.18 (d, J=8.4 Hz, 1H), 7.83˜7.87 (m, 2H), 7.79 (d, J=5.6 Hz, 1H), 7.77 (s, 1H), 7.58 (s, 1H), 7.55 (t, J=4.0 Hz, 1H), 7.15 (t, J=8.8 Hz, 2H), 5.83 (d, J=3.2 Hz, 1H), 3.09 (s, 3H), 2.92 (d, J=4.8 Hz, 3H), 2.73 (s, 3H).

### Step 2: 5-(3-aminophenyl)-2-(4-fluorophenyl)-N-methyl-6-(N-methylmethylsulfonamido)benzofuran-3-carboxamide

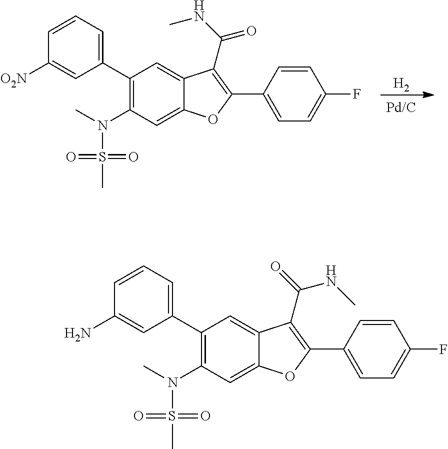

To a solution of 2-(4-fluorophenyl)-N-methyl-6-(N-methylmethylsulfonamido)-5-(3-nitrophenyl)benzofuran-3-carboxamide (1.0 g, 2.01 mmol) in MeOH (30 mL), Pd/C (200 mg) was added and the resulting reaction mixture was allowed to stir under 40 psi of H₂ atmosphere for 24 hours at 25° C. Then the reaction mixture was filtered, and the filtrate was concentrated in vacuo to provide 5-(3-aminophenyl)-2-(4-fluorophenyl)-N-methyl-6-(N-methylmethylsulfonamido)benzofuran-3-carboxamide (846 mg, 89%). ¹H-NMR (DMSO, 400 MHz) δ 8.49 (d, J=4.8 Hz, 1H), 7.94˜7.97 (m, 2H), 7.84 (s, 1H), 7.43 (s, 1H), 7.38 (t, J=9.2 Hz, 2H), 7.03 (t, J=8.0 Hz, 1H), 6.53˜6.58 (m, 3H), 5.09 (s, 2H), 3.13 (d, J=5.6 Hz, 3H), 3.04 (s, 3H), 2.81 (s, 3H). MS (M+H)⁺: 468.

### Step 3: 2-(4-fluorophenyl)-5-(3-iodophenyl)-N-methyl-6-(N-methylmethylsulfonamido)benzofuran-3-carboxamide

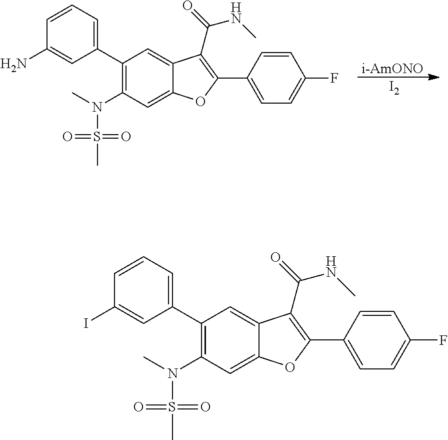

To a stirred solution of 5-(3-aminophenyl)-2-(4-fluorophenyl)-N-methyl-6-(N-methylmethylsulfonamido)benzofuran-3-carboxamide (1.5 g, 3.21 mmol) in MeCN (20 mL) was added I₂ (488.6 mg, 1.93 mmol) and CuI (6 mg) at 0° C., then i-AmONO (394.6 mg, 3.37 mmol) was added dropwise. After the solution was allowed to stir at 25° C. for 6 hours, the mixture was heated to 90° C. for 1 hour. The mixture was diluted with Na₂S₂O₃ and concentrated to remove the organic solvent, and then the residue obtained was extracted with EtOAc. The organic layer was washed with brine, dried over Na₂SO₄ and concentrated in vacuo. The residue obtained was purified using flash column chromatography (PE:EtOAc=10:1) to provide 2-(4-fluorophenyl)-5-(3-iodophenyl)-N-methyl-6-(N-methylmethylsulfonamido)benzofuran-3-carboxamide (1.17 g, 65%). ¹H-NMR (CDCl₃, 400 MHz) δ 7.85˜7.88 (m, 2H), 7.72 (d, J=7.6 Hz, 2H), 7.66 (d, J=8.0 Hz, 1H), 7.53 (s, 1H), 7.38 (d, J=7.6 Hz, 1H), 7.14 (t, J=6.0 Hz, 2H), 5.77 (d, J=4.0 Hz, 1H), 3.06 (s, 3H), 2.92 (d, J=4.8 Hz, 3H), 2.61 (s, 3H). MS (M+H)⁺: 579.

### Step 4—5-(3-(benzo[b]thiophen-2-yl)phenyl)-2-(4-fluorophenyl)-N-methyl-6-(N-methylmethylsulfonamido)benzofuran-3-carboxamide

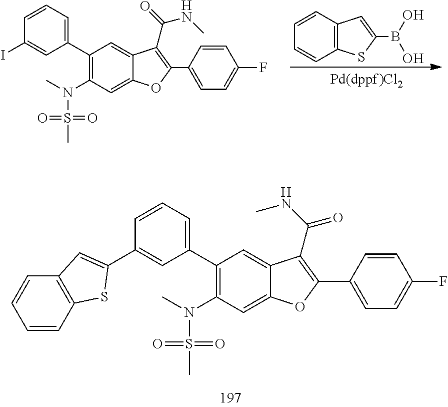

To a degassed solution of 5-(3-aminophenyl)-2-(4-fluorophenyl)-N-methyl-6-(N-methylmethylsulfonamido)benzofuran-3-carboxamide (70 mg, 121.0 umol) and benzo[b]thiophen-2-ylboronic acid (26.1 mg, 145.1 umol) in dry DMF (1.5 mL) were added Pd(dppf)Cl₂ (5 mg) and K₃PO₄ (51.4 mg, 171.2 umol) under N₂. The mixture was heated to 90° C. for about 15 hours. After the reaction mixture was cooled to room temperature, diluted with EtOAc and filtered, the filtrate was washed with H₂O, brine, dried over Na₂SO₄. After concentrated, the crude was purified using prep-TLC (PE:EtOAc=3:1) to provide 5-(3-(benzo[b]thiophen-2-yl)phenyl)-2-(4-fluorophenyl)-N-methyl-6-(N-methylmethylsulfonamido)benzofuran-3-carboxamide (38 mg, 60%). ¹H-NMR (CDCl₃, 400 MHz) δ 7.95˜7.98 (m, 2H), 7.85 (d, J=7.2 Hz, 3H), 7.80 (d, J=7.6 Hz, 1H), 7.76 (d, J=6.8 Hz, 1H), 7.64 (t, J=3.2 Hz, 2H), 7.52 (d, J=7.6 Hz, 1H), 7.44 (d, J=8.0 Hz, 1H), 7.37 (t, J=8.8 Hz, 2H), 7.22 (t, J=8.8 Hz, 2H), 6.04 (d, J=4.4 Hz, 1H), 3.20 (s, 3H), 2.99 (d, J=4.8 Hz, 3H), 2.67 (s, 3H). MS (M+H)⁺: 585.

The following compounds of the present invention were prepared using the method described in Example 351 and substituting the appropriate reactants and/or reagents.

| Com- | | | MS |
|---|---|---|---|
| pound | Structure | NMR | (M + H)⁺ |
| 352 | 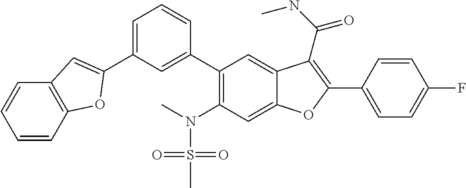 | ¹H-NMR (DMSO, 400 MHz) δ 8.53 (d, J = 4.8 Hz, 1H), 8.02 (d, J = 6.8 Hz, 1H), 8.00 (d, J = 5.6 Hz, 2H), 7.93 (d, J = 7.6 Hz, 1H), 7.62~7.67 (m, 3H), 7.58 (t, J = 7.6 Hz, 1H), 7.48 (t, J = 6.0 Hz, 2H), 7.39~7.45 (m, 2H), 7.24~7.33 (m, 2H), 3.11 (s, 3H), 2.96 (s, 3H), 2.80 (d, J = 4.4 Hz, 3H). | 569 |
| 353 | 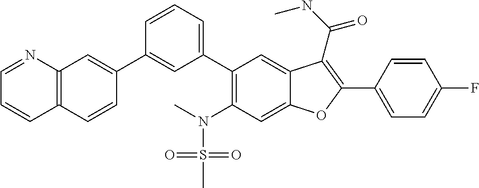 | ¹H-NMR (CDCl₃, 400 MHz) δ 9.19 (s, 1H), 8.71 (d, J = 7.2 Hz, 2H), 8.09~8.20 (m, 2H), 7.88~7.91 (m, 3H), 7.77~7.82 (m, 3H), 7.53~7.60 (m, 3H), 7.11~7.16 (m, 2H), 6.04 (s, 1H), 3.12 (s, 3H), 2.93 (d, J = 4.4 Hz, 3H), 2.72 (s, 3H). | 580 |
| 354 | 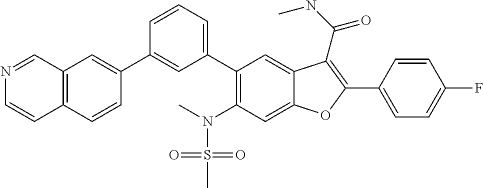 | ¹H-NMR (CDCl₃, 400 MHz) δ 9.67 (s, 1H), 8.60 (s, 2H), 8.43 (d, J = 8.8 Hz, 1H), 8.15 (m, 2H), 7.95~8.03 (m, 4H), 7.78 (d, J = 7.2 Hz, 1H), 7.58~7.68 (m, 3H), 7.22~7.27 (m, 2H), 5.91 (s, 1H) 3.12 (s, 3H), 3.01 (d, J = 4.8 Hz, 3H), 2.96 (s, 3H). | 580 |
| 356 | 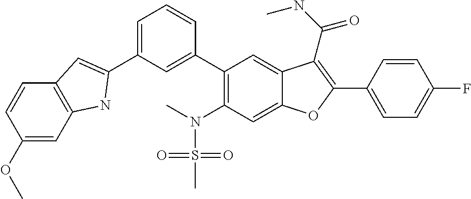 | ¹H-NMR (CDCl₃, 400 MHz) δ 8.94 (s, 1H), 7.89~7.86 (m, 2H), 7.82 (s, 1H), 7.79 (s, 1H), 7.62 (d, J = 7.6 Hz, 1H), 7.46 (s, 1H), 7.43~7.40 (m, 2H), 7.26 (d, J = 7.6 Hz, 1H), 7.13 (t, J = 8.4 Hz, 2H), 6.83 (s, 1H), 6.70 (d, J = 8.4 Hz, 2H), 5.83 (d, J = 4.0 Hz, 1H), 3.78 (s, 3H), 2.91 (d, J = 6.8 Hz, 9H). | 598 |
| 357 | 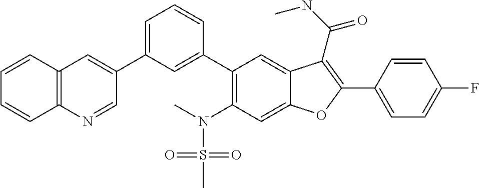 | ¹H NMR (CDCl₃, 400 MHz) δ 9.18 (s, 1H), 8.35 (s, 1H), 8.06 (d, J = 8.8 Hz, 1H), 7.67~7.86 (m, 3H), 7.66 (s, 1H), 7.64 (d, J = 1.2 Hz, 1H), 7.52~7.56 (m, 2H), 7.46~7.52 (m, 3H), 7.44 (d, J = 1.6 Hz, 1H), 7.13 (t, J = 8.8 Hz, 2H), 5.94 (d, J = 4.8 Hz, 1H), 3.09 (s, 3H), 2.91 (d, J = 4.8 Hz, 3H), 2.66 (s, 3H). | 580 |
| 358 | 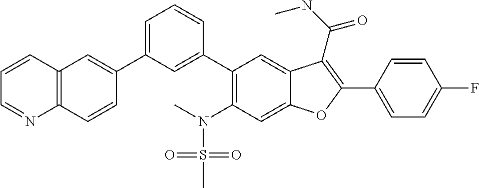 | ¹H-NMR (CDCl₃, 400 MHz) δ 8.85~8.88 (m, 1H), 8.05~8.20 (m, 2H), 7.99 (d, J = 1.6 Hz, 1H), 7.97 (d, J = 1.6 Hz, 1H), 7.87~7.91 (m, 2H), 7.80 (d, J = 9.2 Hz, 2H), 7.69 (d, J = 8.0 Hz, 1H), 7.56 (s, 1H), 7.52 (t, J = 7.6 Hz, 1H), 7.37~7.43 (m, 2H), 7.14 (t, J = 8.8 Hz, 2H), 5.80 (d, J = 4.4 Hz, 1H), 3.10 (s, 3H), 2.92 (d, J = 4.8 Hz, 3H), 2.64 (s, 3H). | 580 |
| 359 | 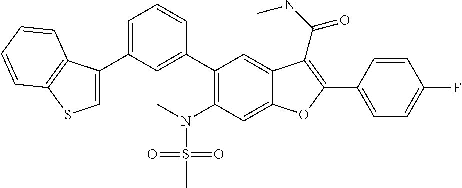 | ¹H NMR (CDCl₃, 400 MHz) δ 7.92~7.95 (m, 4H), 7.83 (s, 1H), 7.67 (s, 1H), 7.60~7.62 (m, 2H), 7.53~7.57 (m, 1H), 7.45~7.49 (m, 2H), 7.36~7.39 (m, 2H), 7.16~7.20 (m, 2H), 5.84 (s, 1H), 3.19 (s, 3H), 2.97 (d, J = 4.8 Hz, 3H), 2.62 (s, 3H). | 585 |
| 360 | 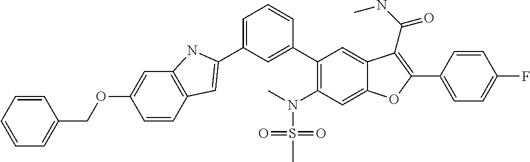 | ¹H NMR (CDCl₃, 400 MHz) δ 8.92 (s, 1H), 7.92~7.96 (m, 2H), 7.87 (s, 2H), 7.66~7.68 (m, 1H), 7.53 (s, 1H), 7.45~7.49 (m, 3H), 7.30~7.39 (m, 3H), 7.17~7.21 (m, 2H), 6.97 (s, 1H), 6.83~6.85 (m, 1H), 6.76 (s, 1H), 5.84 (s, 1H), 5.10 (s, 2H), 2.93~2.98 (m, 9H). | 674 |
| 361 | 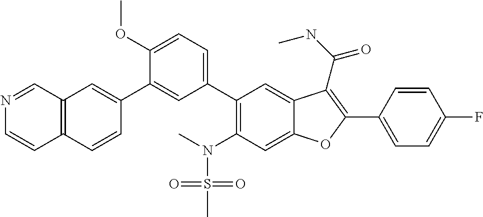 | ¹H-NMR (CDCl₃, 400 MHz) δ 9.25 (s, 1H), 8.46 (d, J = 5.6 Hz, 1H), 8.16 (s, 1H), 7.94 (d, J = 8.4 Hz, 1H), 7.86~7.90 (m, 2H), 7.81 (d, J = 10.8 Hz, 2H), 7.64 (d, J = 4.8 Hz, 1H), 7.51 (t, J = 2.0 Hz, 2H), 7.42~7.45 (m, 1H), 7.14 (t, J = 8.8 Hz, 2H), 7.05 (d, J = 8.4 Hz, 1H), 5.76 (d, J = 3.6 Hz, 1H), 3.85 (s, 3H), 3.09 (s, 3H), 2.92 (d, J = 4.8 Hz, 3H), 2.75 (s, 3H). | 610 |

### Example 362

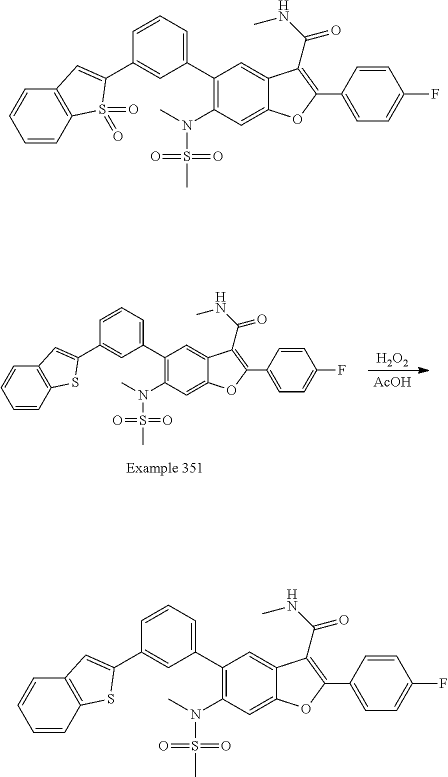

To a solution of the compound of Example 351 (100 mg, 0.38 mmol) in 10 mL of acetic acid was added H₂O₂ (2 mL) and the resulting reaction mixture was heated to 65° C. and allowed to stir at this temperature for 3 hours. The reaction was then was quenched with aq. Na₂SO₃ and extracted with EtOAc. The organic phase was washed with H₂O and brine, dried over MgSO₄, filtered and concentrated in vacuo. The residue obtained was purified using preparative HPLC to provide the target compound (45 mg, 28%). ¹H NMR: (CDCl₃, 400 MHz) 7.92 (s, 1H), 7.86˜7.90 (m, 2H), 7.74˜7.76 (s, 2H), 7.69˜7.70 (m, 1H), 7.43˜7.56 (m, 5H), 7.34˜7.38 (m, 2H), 7.14 (t, J=8.8 Hz, 2H), 5.84 (s, 1H), 3.18 (s, 3H), 2.93 (d, J=4.8 Hz, 3H), 2.54 (s, 3H). MS (M+H)⁺: 617.

### Example 363

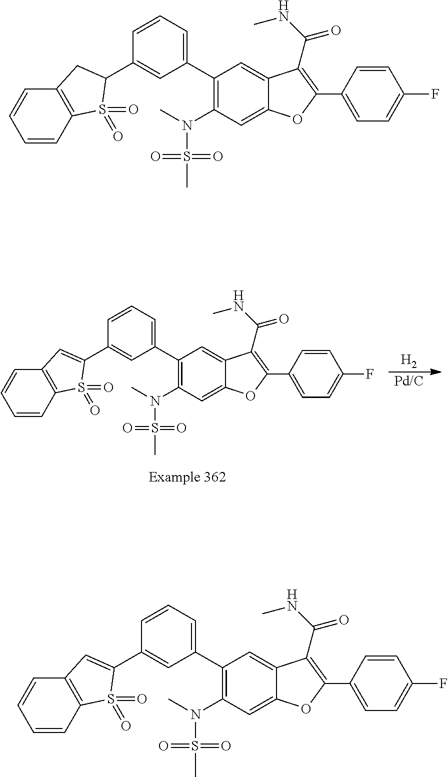

To a solution of the compound of Example 362 (30 mg, 0.13 mmol) in 10 mL of MeOH, was added Pd/C (10 mg), and the resulting reaction was placed under H₂ atmosphere (40 Psi) and allowed to stir at room temperature for 24 hours. The reaction mixture was then filtered and concentrated in vacuo, and the residue obtained was purified using preparative HPLC to provide Compound 209 (20 mg, 85%). ¹H-NMR (CDCl₃, 400 MHz) δ 7.86˜7.90 (m, 2H), 7.72˜7.73 (m, 2H), 7.54˜7.58 (m, 2H), 7.39˜7.46 (m, 6H), 7.11˜7.16 (m, 2H), 5.77˜5.78 (m, 1H), 4.68 (t, J=8.2 Hz, 1H), 3.64 (d, J=8.2 Hz, 2H), 3.09 (s, 3H), 2.93 (d, J=4.8 Hz, 3H), 2.46 (s, 3H). MS (M+H)⁺: 619.

### Example 364

### 2-(4-fluorophenyl)-5-(3-(isoquinolin-6-yl)phenyl)-N-methyl-6-(N-methylmethylsulfonamido)benzofuran-3-carboxamide

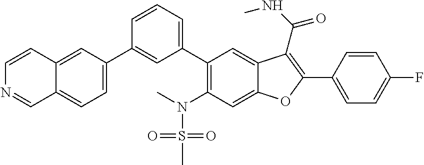

### Step 1: 2-(4-fluorophenyl)-N-methyl-6-(N-methylmethylsulfonamido)-5-(3-(4,4,5,5-tetramethyl-1,3,2-dioxaborolan-2-yl)phenyl)benzofuran-3-carboxamide

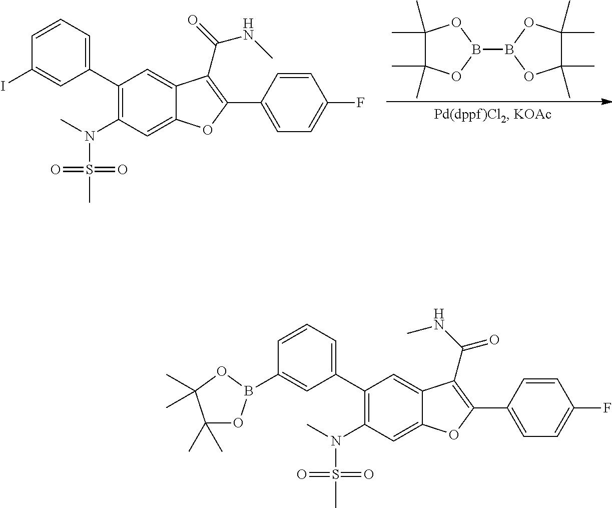

To a degassed solution of 2-(4-fluorophenyl)-5-(3-iodophenyl)-N-methyl-6-(N-methylmethylsulfonamido)benzofuran-3-carboxamide (Prepared as described in Example 7, Step 3, 200 mg, 0.346 mmol) and pinacol diborane (132 mg, 0.519 mmol) in dry DMF (1.5 mL) was added Pd(dppf)Cl₂ (10 mg) and KOAc (102 mg, 1.04 mmol). The mixture was placed under N₂ atmosphere, then heated to 90° C. and allowed to stir at this temperature for about 15 hours. The reaction mixture was cooled to room temperature, filtered, and the filtrate was washed with H₂O, brine, dried over Na₂SO₄, filtered and concentrated in vacuo to provide 2-(4-fluorophenyl)-N-methyl-6-(N-methylmethylsulfonamido)-5-(3-(4,4,5,5-tetramethyl-1,3,2-dioxaborolan-2-yl)phenyl)benzofuran-3-carboxamide (200 mg, 100%), which was used without further purification. ¹H-NMR (CDCl₃, 400 MHz) δ 7.88˜7.92 (m, 2H), 7.75˜7.78 (m, 2H), 7.72 (s, 1H), 7.56 (s, 1H), 7.49˜7.52 (m, 1H), 7.37˜7.41 (m, 1H), 7.11˜7.15 (m, 2H), 5.81˜5.82 (m, 1H), 3.05 (s, 3H), 2.93 (d, J=4.8 Hz, 3H), 2.51 (s, 3H), 1.29 (s, 12H). MS (M+H)⁺: 579.

### Step 2: 2-(4-fluorophenyl)-5-(3-(isoquinolin-6-yl)phenyl)-N-methyl-6-(N-methylmethylsulfonamido)benzofuran-3-carboxamide

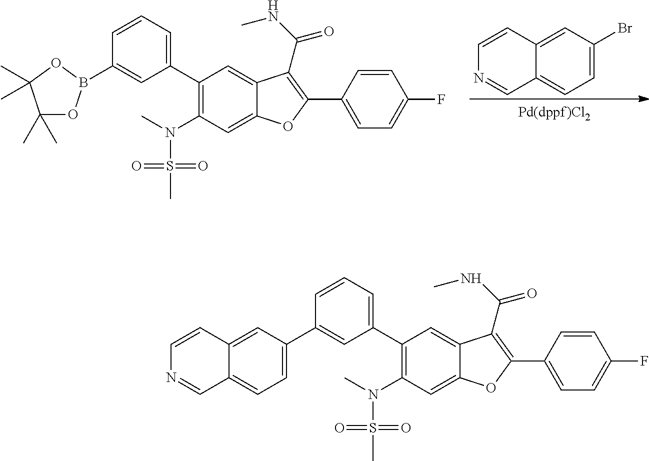

To a degassed solution of 2-(4-fluorophenyl)-N-methyl-6-(N-methylmethylsulfonamido)-5-(3-(4,4,5,5-tetramethyl-1,3,2-dioxaborolan-2-yl)phenyl)benzofuran-3-carboxamide (90 mg, 0.189 mmol) and 6-bromo-isoquinoline (51 mg, 0.246 mmol) in dry DMF (1.5 mL) was added Pd(dppf)Cl₂(20 mg) and K₃PO₄ (81 mg, 0.381 mmol) under N₂. The mixture was heated to 100° C. for about 15 hours. The reaction mixture was cooled to room temperature and filtered. The filtrate was washed with H₂O, brine, dried over Na₂SO₄, filtered and concentrated in vacuo. The residue obtained was purified using prep-TLC (PE:EtOAc=2:1) to provide 2-(4-fluorophenyl)-5-(3-(isoquinolin-6-yl)phenyl)-N-methyl-6-(N-methylmethylsulfonamido)benzofuran-3-carboxamide (85 mg, 93%). ¹H-NMR (CDCl₃, 400 MHz) δ 9.62 (s, 1H), 8.46 (d, J=6.0 Hz, 1H), 8.38 (s, 1H), 8.31˜8.33 (m, 1H), 8.21˜8.23 (m, 1H), 8.15 (d, J=6.0 Hz, 1H), 7.98 (s, 1H), 7.81=7.85 (m, 3H), 7.71˜7.72 (m, 1H), 7.51˜7.60 (m, 3H), 7.12˜7.19 (m, 2H), 6.02˜6.03 (m, 1H), 3.02 (s, 3H), 2.89˜2.92 (m, 6H). MS (M+H)⁺: 580.

The following compound of the present invention was prepared using the method described in Example 364 and substituting the appropriate reactants and/or reagents.

| | | | MS |
|---|---|---|---|
| Compound | Structure | NMR | (M + H)⁺ |
| 365 | 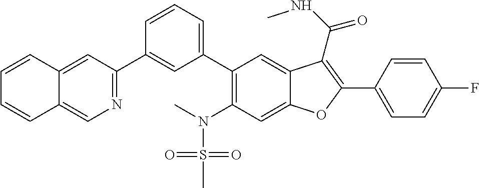 | ¹H-NMR (CDCI1, 400 MHz) δ 9.79 (s, 1H), 8.50 (s, 1H), 8.31 (d, J = 8.0 Hz, 1H), 8.04~8.12 (m, 3H), 7.85~7.92 (m, 4H), 7.80 (s, 1H), 7.64~7.65 (m, 2H), 7.52 (s, 1H), 7.11~7.15 (m, 2H), 6.43~6.44 (m, 1H), 3.02 (s, 3H), 2.94 (d, J = 4.8 Hz, 3H), 2.87 (s, 3H). | 580 |

### Example 366

### 5-(3-(1H-indol-2-yl)phenyl)-2-(4-fluorophenyl)-N-methyl-6-(N-methylmethylsulfonamido)benzofuran-3-carboxamide

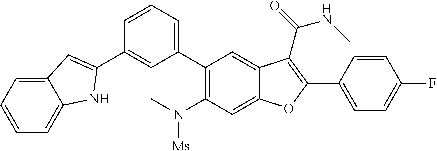

### Step 1: 2-(4-fluorophenyl)-N-methyl-6-(N-methylmethylsulfonamido)-5-(3-(1-((2-(trimethylsilyl)ethoxy)methyl)-1H-indol-2-yl)phenyl)benzofuran-3-carboxamide

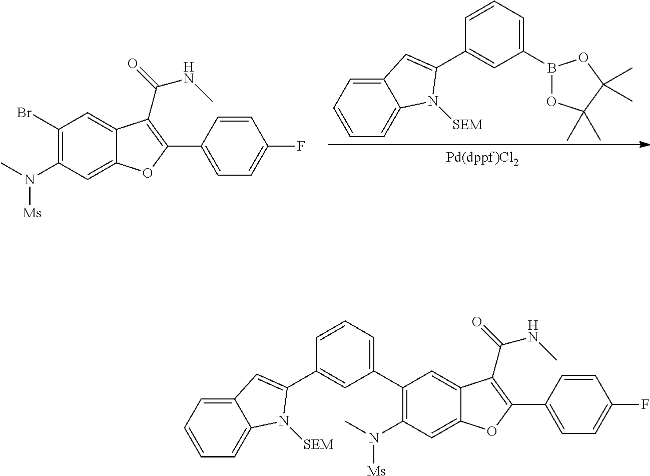

### 5-bromo-2-(4-fluorophenyl)-N-methyl-6-(N-methylmethylsulfonamido)-1-benzofuran-3-carboxamide (prepared as described in Example 1, Step 11) was converted to 2-(4-fluorophenyl)-N-methyl-6-(N-methylmethylsulfonamido)-5-(3-(1-((2-(trimethylsilyl)ethoxy)methyl)-1H-indol-2-yl)phenyl)benzofuran-3-carboxamide (120 mg, 53.4%) using the method described in Example 1, Step 1. ¹H-NMR (CDCl₃, 400 MHz) δ 8.07˜8.03 (m, 2H), 7.93 (s, 1H), 7.82˜7.80 (m, 2H), 7.74˜7.72 (m, 2H), 7.65˜7.60 (m, 2H), 7.37˜7.35 (m, 2H), 7.32˜7.27 (m, 3H), 6.77 (s, 1H), 6.05 (d, J=4.4 Hz, 1H), 5.61 (s, 2H), 3.62 (t, J=8.4 Hz, 2H), 3.31 (s, 3H), 3.08 (d, J=4.8 Hz, 3H), 2.72 (s, 3H), 0.95 (t, J=8.4 Hz, 2H), 0.00 (s, 9H). MS (M+H)⁺: 698.

### Step 2: 5-(3-(1H-indol-2-yl)phenyl)-2-(4-fluorophenyl)-N-methyl-6-(N-methylmethylsulfonamido)benzofuran-3-carboxamide

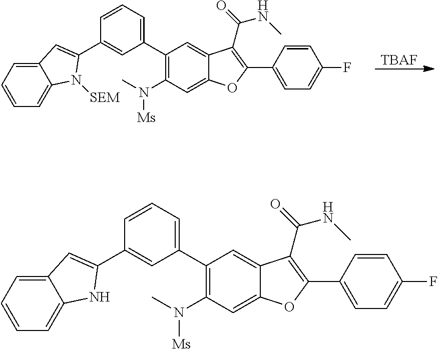

2-(4-fluorophenyl)-N-methyl-6-(N-methylmethylsulfonamido)-5-(3-(1-((2-(trimethylsilyl)ethoxy)methyl)-1H-indol-2-yl)phenyl)benzofuran-3-carboxamide (60 mg, 0.86 mmol) and TBAF (67.44 mg, 2.57 mmol) in DMF (2 mL) was added to a flask, ethylene diamine (25.83 mg, 0.95 mmol) was added. The mixture was purged with nitrogen and heated at 80° C. for about 15 hours. The mixture was diluted with EtOAc and washed with 0.1 M HCl. The phases were separated, and the organic phase was washed with water and brine, dried over Na₂SO₄, filtered and concentrated in vacuo. The resulting residue was purified using preparative TLC to provide 5-(3-(1H-indol-2-yl)phenyl)-2-(4-fluorophenyl)-N-methyl-6-(N-methylmethylsulfonamido)benzofuran-3-carboxamide (20 mg, 41.4%). ¹H-NMR (CDCl₃, 400 MHz) δ 9.30 (s, 1H), 7.94 (d, J=8.8 Hz, 3H), 7.83 (s, 1H), 7.74 (d, J=8.0 Hz, 1H), 7.65 (t, J=7.2 Hz, 1H), 7.52˜7.47 (m, 2H), 7.43 (d, J=8.0 Hz, 1H), 7.35 (d, J=6.8 Hz, 1H), 7.22˜7.17 (m, 3H), 7.14˜7.10 (m, 1H), 6.85 (s, 1H), 6.09 (d, J=4.4 Hz, 1H), 2.99 (s, 3H), 2.97 (d, J=4.0 Hz, 3H), 2.92 (s, 3H). MS (M+H)⁺: 568.

The following compounds of the present invention were prepared using the method described in Example 366 and substituting the appropriate reactants and/or reagents.

| Com- | | | MS |
|---|---|---|---|
| pound | Structure | NMR | (M + H)⁺ |
| 367 | 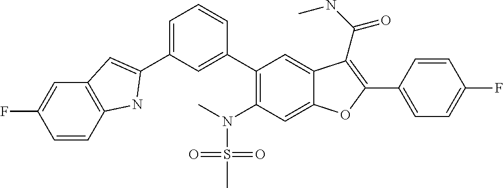 | ¹H-NMR (CDCl₃, 400 MHz) δ 9.10 (s, 1H), 7.89~7.84 (m, 4H), 7.66 (d, J = 8.0 Hz, 1H), 7.45 (t, J = 5.6 Hz, 2H), 7.33 (d, J = 7.2 Hz, 1H), 7.28~7.25 (m, 1H), 7.17~7.12 (m, 3H), 6.88~6.83 (m, 1H), 6.73 (d, J = 1.2 Hz, 1H), 5.84 (d, J = 4.4 Hz, 1H), 2.96 (s, 3H), 2.93 (d, J = 4.8 Hz, 3H), 2.88 (s, 3H). | 586 |
| 368 | 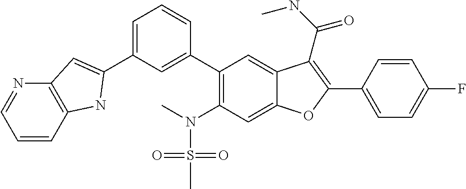 | ¹H-NMR (CDCl₃, 400 MHz) δ 8.26 (d, J = 6.0 Hz, 1H), 8.17 (d, J = 8.0 Hz, 2H), 7.95~7.99 (m, 3H), 7.87 (s, 1H), 7.70 (d, J = 6.4 Hz, 1H), 7.57 (d, J = 16 Hz, 2H), 7.50 (d, J = 7.2 Hz, 2H), 7.19~7.24 (m, 2H), 6.98 (s, 1H), 6.52 (s, 1H), 2.99~3.99 (m, 9H). | 569 |
| 369 | 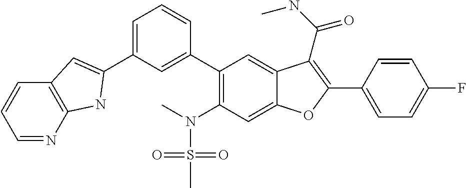 | ¹H-NMR (CDCl₃, 400 MHz) δ 14.50 (s, 1H), 8.30 (d, J = 4.4 Hz, 1H), 8.03~8.05 (m, 1H), 7.84~7.95 (m, 4H), 7.81 (s, 1H), 7.55~7.86 (m, 2H), 7.48~7.50 (m, 1H), 7.30~7.32 (m, 1H), 7.12~7.16 (m, 2H), 7.01 (s, 1H), 5.94 (d, J = 4.8 Hz, 1H), 3.16 (s, 3H), 2.93 (d, J = 4.8 Hz, 3H), 2.66 (s, 3H). | 569 |
| 370 | 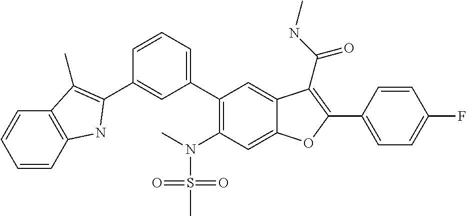 | ¹H-NMR (DMSO, 400 MHz) δ 11.18 (s, 1H), 8.55 (d, J = 4.8 Hz, 1H), 8.01~8.04 (m, 3H), 7.74 (s, 1H), 7.61~7.70 (m, 2H), 7.53~7.61 (m, 2H), 7.36~7.46 (m, 4H), 7.09~7.13 (m, 1H), 7.00~7.04 (m, 1H), 3.16 (s, 3H), 2.95 (s, 3H), 2.82 (d, J = 4.4 Hz, 3H), 2.45 (s, 3H). | 582 |
| 371 | 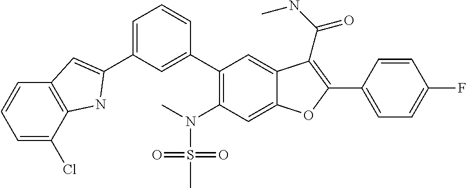 | ¹H-NMR (CDCl₃, 400 MHz) δ 9.15 (s, 1H), 7.96~8.01 (m, 3H), 7.93 (s, 1H), 7.78 (d, J = 7.6 Hz, 1H), 7.59 (s, 1H), 7.54~7.57 (m, 1H), 7.45 (d, J = 8.0 Hz, 1H), 7.20~7.26 (m, 3H), 7.07 (d, J = 8.0 Hz, 1H), 6.91 (s, 1H), 5.89 (d, J = 3.6 Hz, 1H), 3.02~3.03 (m, 9H). | 602 |
| 372 | 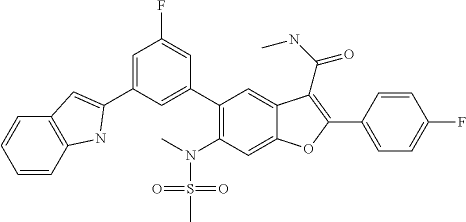 | ¹H-NMR (CDCl₃, 400 MHz) δ 9.13 (s, 1H), 7.86~7.90 (m, 3H), 7.71 (s, 1H), 7.56~7.58 (m, 1H), 7.48 (s, 1H), 7.36~7.38 (m, 2H), 7.12~7.17 (m, 3H), 7.01~7.06 (m, 2H), 6.78 (s, 1H), 5.86 (s, 1H), 2.94~2.99 (m, 9H). | 586 |
| 373 | 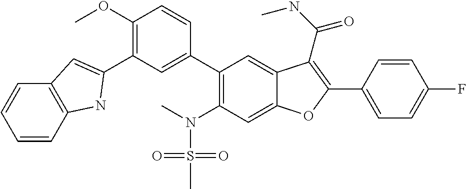 | ¹H-NMR (CDCl₃, 400 MHz) δ 9.74 (s, 1H), 7.98~8.01 (m, 2H), 7.87 (s, 1H), 7.64 (d, J = 10.8 Hz, 2H), 7.41~7.47 (m, 3H), 7.19~7.26 (m, 3H), 7.11~7.16 (m, 2H), 6.98 (s, 1H), 5.88 (d, J = 4.8 Hz, 1H), 4.11 (s, 3H), 3.15 (s, 3H), 3.02 (d, J = 4.8 Hz, 3H), 2.82 (s, 3H). | 598 |
| 374 | 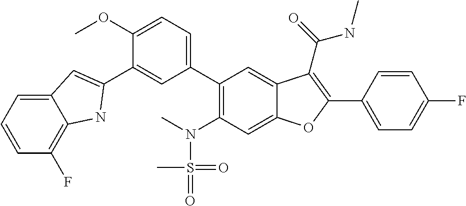 | ¹H-NMR (CDCl₃, 400 MHz) δ 9.79 (s, 1H), 7.88~7.90 (m, 3H), 7.87 (s, 1H), 7.77 (s, 1H), 7.34~7.36 (m, 2H), 7.14 (s, 2H), 6.91~6.94 (m, 1H), 6.82~6.90 (m, 2H), 6.79~6.81 (m, 1H), 5.80 (s, 1H), 4.02 (s, 3H), 3.39 (s, 3H), 3.20 (d, J = 4.8 Hz, 3H), 2.91 (s, 3H). | 616 |
| 375 | 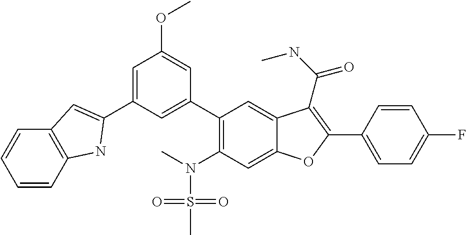 | ¹H-NMR (CDCl₃, 400 MHz) δ 9.00 (s, 1H), 7.85~7.88 (m, 2H), 7.81 (s, 1H), 7.56 (d, J = 6.8 Hz, 1H), 7.46~7.49 (m, 2H), 7.34 (d, J = 8.0 Hz, 1H), 7.26 (s, 1H), 7.10~7.14 (m, 3H), 7.02~7.05 (m, 1H), 6.90 (s, 1H), 6.77 (s, 1H), 5.88 (s, 1H), 3.86 (s, 3H), 2.91~2.95 (m, 9H). | 598 |
| 376 | 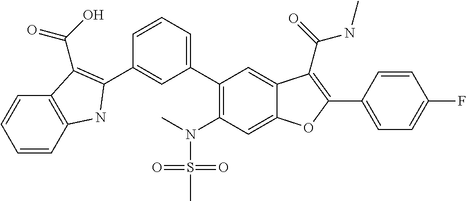 | ¹H-NMR (CDCl₃, 400 MHz) δ 8.03~8.06 (m, 1H), 7.88 (br, 2H), 7.70 (br, 2H), 7.63 (br, 2H), 7.46~7.48 (m, 2H), 7.32~7.34 (m, 1H), 7.10~7.13 (m, 4H), 3.06 (s, 3H), 2.84 (s, 3H), 2.72 (s, 3H). | 612 |
| 377 | 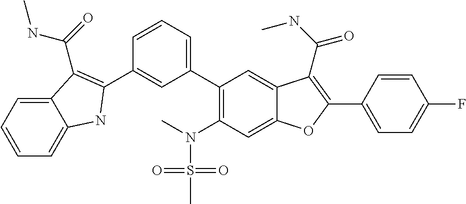 | ¹H-NMR (CDCl₃, 400 MHz) δ 9.57 (s, 1H), 8.07 (d, J = 7.6 Hz, 1H), 7.92~7.98 (m, 4H), 7.81~7.83 (m, 1H), 7.52~7.58 (m, 2H), 7.43~7.48 (m, 2H), 7.21~7.27 (m, 4H), 6.05~6.06 (m, 1H), 5.76~5.78 (m, 1H), 3.15 (s, 3H), 3.01 (d, J = 4.8 Hz, 3H), 2.96~2.97 (m, 6H). | 625 |
| 378 | 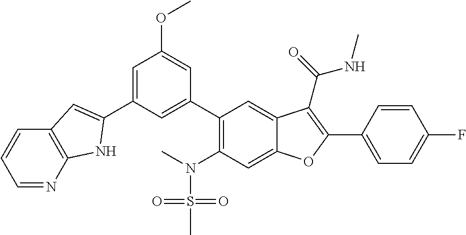 | ¹H-NMR (CDCl₃, 400 MHz) δ 8.32 (d, J = 7.6 Hz, 1H), 8.01 (d, J = 5.6 Hz, 1H), 7.89~7.93 (m, 2H), 7.81 (s, 1H), 7.55 (s, 2H), 7.41 (s, 1H), 7.32 (m, 1H), 7.12~7.16 (m, 2H), 7.15 (s, 1H), 7.09 (s, 1H), 6.99 (s, 1H), 5.96 (s, 1H), 3.91 (s, 3H), 3.12 (s, 3H), 2.95 (d, J = 4.8 Hz, 3H), 2.75 (s, 3H). | 599 |
| 379 | 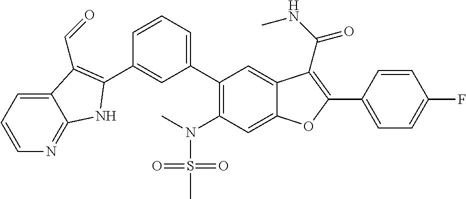 | ¹H-NMR (CDCl₃, 400 MHz) δ 9.98 (s, 1H), 8.54~8.57 (m, 1H), 8.23~8.15 (m, 1H), 7.81~7.84 (m, 2H), 7.70~7.75 (m, 2H), 7.61~7.66 (m, 1H), 7.52~7.56 (m, 3H), 7.17~7.20 (m, 1H), 7.07~7.12 (m, 2H), 3.05 (s, 3H), 2.85 (s, 3H), 2.79 (s, 3H). | 597 |
| 380 | 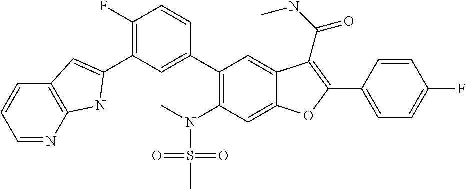 | ¹H-NMR (CDCl₃, 400 MHz) δ 9.75 (s, 1H), 8.29~8.31 (m, 1H), 8.01~8.04 (m, 1H), 7.91~7.95 (m, 3H), 7.88 (s, 1H), 7.55 (s, 1H), 7.37~7.41 (m, 1H), 7.27~7.30 (m, 1H), 7.20 (t, J = 8.8 Hz, 2H), 7.04~7.07 (m, 1H), 6.95 (s, 1H), 5.84 (d, J = 4.4 Hz, 1H), 3.02 (s, 3H), 2.98 (d, J = 4.8 Hz, 3H), 2.97 (s, 3H). | 587 |

### Example 381

### 5-(3-(3-chloro-1H-indol-2-yl)phenyl)-2-(4-fluorophenyl)-N-methyl-6-N-methylmethylsulfonamido)benzofuran-3-carboxamide

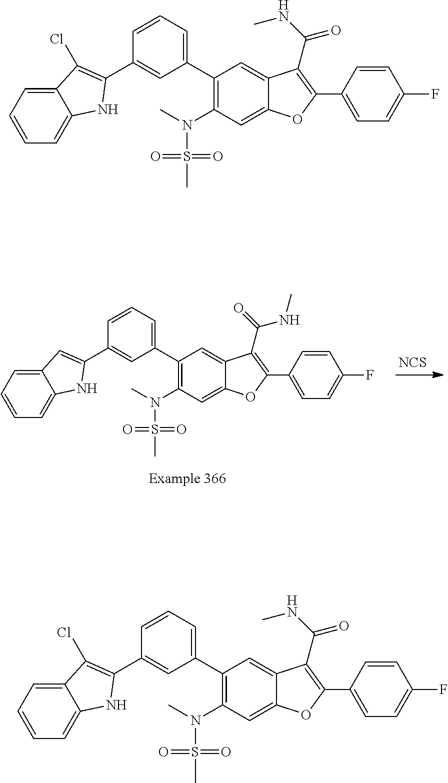

To a solution of Example 366 (50 mg, 0.088 mmol) in 2 mL of DMF, was added NCS (15 mg, 0.088 mmol), and the resulting reaction was allowed to stir under N₂ atmosphere for 4 hours at 25° C. The reaction mixture was concentrated in vacuo and the resulting residue was diluted EtOAc. The resulting solution was washed with brine, dried over Na₂SO₄, filtered and concentrated in vacuo. The resulting residue was purified using prep-TLC (PE:EtOAc=2:1) to provide the title compound (20 mg, 50%) as a white solid. ¹H-NMR (CDCl₃, 400 MHz) δ 9.29 (s, 1H), 7.97 (d, J=7.6 Hz, 1H), 7.83˜7.86 (m, 2H), 7.78 (s, 1H), 7.57 (d, J=8.0 Hz, 1H), 7.49 (t, J=7.6 Hz, 1H), 7.41 (s, 1H), 7.33 (t, J=5.6 Hz, 2H), 7.17 (d, J=7.6 Hz, 1H), 7.09˜7.15 (m, 3H), 5.92 (d, J=4.4 Hz, 1H), 2.97 (s, 3H), 2.87 (d, J=4.8 Hz, 3H), 2.85 (s, 3H).

MS (M+H)⁺: 602.

### Example 382

### 5-(3-(3-bromo-1H-indol-2-yl)phenyl)-2-(4-fluorophenyl)-N-methyl-6-(N-methylmethylsulfonamido)benzofuran-3-carboxamide

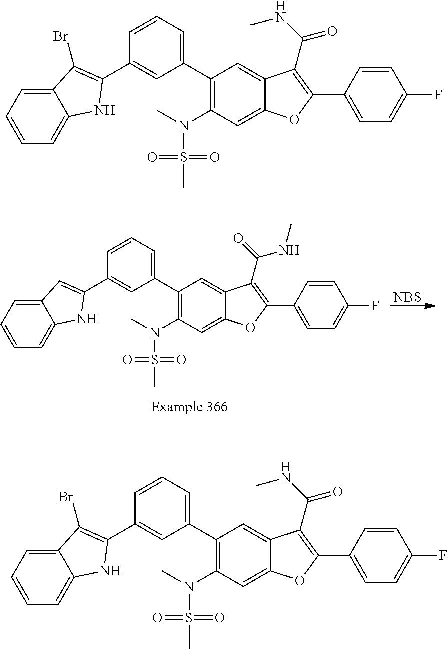

To a solution of the compound of Example 366 (50 mg, 0.088 mmol) in 3 mL of DMF, was added NBS (16 mg, 0.088 mmol) and the resulting reaction was heated to 75° C. and allowed to stir at this temperature for 4 hours. The reaction mixture was cooled to room temperature and concentrated in vacuo. The resulting residue was diluted with EtOAc and the resulting solution was washed with brine, dried over Na₂SO₄, filtered and concentrated in vacuo. The residue obtained was purified using prep-TLC (PE:EtOAc=2:1) to provide the title compound (40 mg, 89%) as a white solid. ¹H-NMR (CDCl₃, 400 MHz) δ 9.38 (s, 1H), 8.02 (d, J=8.0 Hz, 1H), 7.94 (s, 1H), 7.88˜7.94 (m, 2H), 7.84 (s, 1H), 7.53 (t, J=7.6 Hz, 2H), 7.46 (d, J=4.8 Hz, 1H), 7.35˜7.40 (m, 2H), 7.11˜7.15 (m, 4H), 5.80 (s, 1H), 3.04 (s, 3H), 2.94 (d, J=5.2 Hz, 3H), 2.87 (s, 3H). MS (M+H)⁺: 646.

### Example 383

### 2-(4-fluorophenyl)-5-(3-(3-(hydroxymethyl)-1H-pyrrolo[2,3-b]pyridin-2-yl)phenyl)-N-methyl-6-(N-methylmethylsulfonamido)benzofuran-3-carboxamide

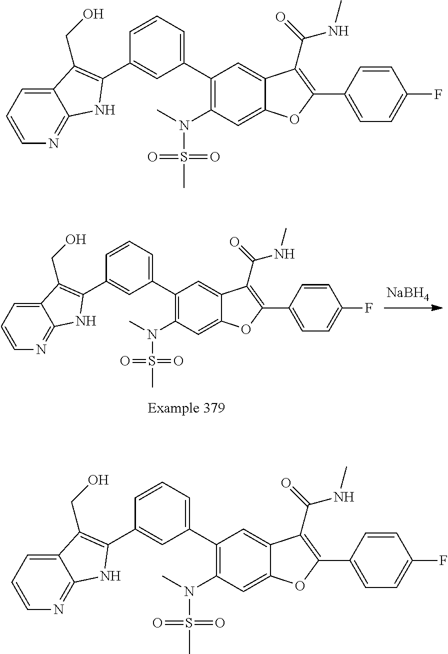

To a solution of the compound of Example 379 (50 mg, 0.084 mmol) in MeOH (5 mL) was added NaBH₄ (17 mg, 0.5 mmol) and the resulting reaction was allowed to stir at room temperature for 2 hours. The reaction mixture was diluted with water and extracted with dichloromethane and the organic extract was dried over Na₂SO₄, filtered and concentrated in vacuo to provide the title compound (20 mg, 40%). ¹H-NMR (CDCl₃, 400 MHz) δ 10.1510.25 (m, 1H), 8.22 (d, J=3.6 Hz, 1H), 8.02˜8.04 (m, 1H), 7.88˜7.91 (m, 3H), 7.82 (s, 1H), 7.70˜7.72 (m, 1H), 7.50˜7.54 (m, 1H), 7.48 (s, 1H), 7.40˜7.42 (m, 1H), 7.12˜7.16 (m, 2H), 7.05˜7.08 (m, 1H), 5.93˜5.98 (m, 1H), 4.92 (s, 2H), 2.96 (s, 3H), 2.91˜2.93 (m, 6H).

### Example 384

### 2-(4-fluorophenyl)-6-(N-(3-hydroxypropyl)methylsulfonamido)-N-methyl-5-(3-(oxazolo[4,5-b]pyridin-2-yl)phenyl)benzofuran-3-carboxamide

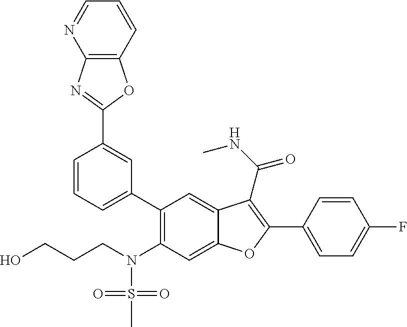

### Step 1: 5-bromo-2-(4-fluorophenyl)-6-(methylsulfonamido)benzofuran-3-carboxylic acid

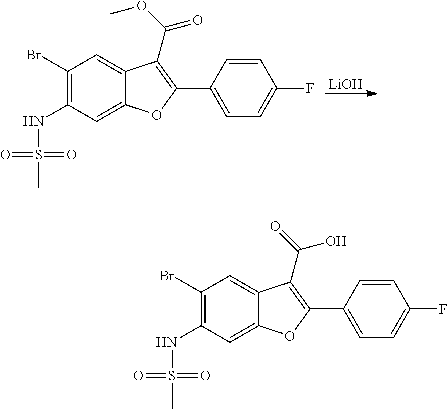

To a solution of methyl 5-bromo-2-(4-fluorophenyl)-6-(methylsulfonamido)benzofuran-3-carboxylate (prepared as described in Example 1, Step 8, 0.5 g, 1.13 mmol) in dioxane (3 mL) and water (1 mL) was LiOH.H₂O (0.24 g, 5.65 mmol). The resulting reaction was heated to 80° C. and allowed to stir at this temperature for 2 hours. The reaction mixture was cooled to room temperature and adjusted to pH=6-7 using conc. HCl. The resulting solution was extracted with EtOAc, and the organic phase was dried over anhydrous Na₂SO₄, filtered and concentrated in vacuo to provide 5-bromo-2-(4-fluorophenyl)-6-(methylsulfonamido)benzofuran-3-carboxylic acid (0.4 g. 87%) as a white solid. ¹H-NMR (DMSO, 400 MHz) δ 13.49 (s, 1H), 9.67 (s, 1H), 8.30 (s, 1H), 8.12˜8.17 (m, 2H), 7.87 (s, 1H), 7.45˜7.50 (m, 2H), 3.16 (s, 3H). MS (M+H)⁺: 428.

### Step 2: 5-bromo-2-(4-fluorophenyl)-N-methyl-6-(methylsulfonamido)benzofuran-3-carboxamide

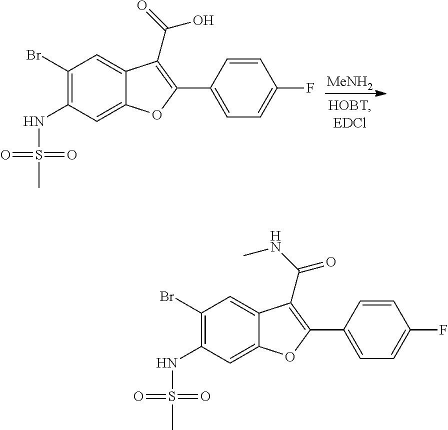

To a solution of 5-bromo-2-(4-fluorophenyl)-6-(methylsulfonamido)benzofuran-3-carboxylic acid (420 mg, 0.77 mmol) in DMF (10 mL) was added EDCI (295 mg, 1.57 mmol) and HOBT (104 mg, 0.77 mmol), and the resulting reaction was allowed to stir at room temperature for 3 hours. CH₃NH₂.HCl (102 mg, 1.54 mmol) and Et₃N (3 mL) were then added to the reaction mixture and the resulting reaction was allowed to stir at room temperature for an additional 8 hours. The reaction mixture was then concentrated in vacuo and the residue obtained was diluted with EtOAc. The resulting solution was washed with HCl (1 N) and NaOH (1 N), dried over Na₂SO₄, filtered and concentrated in vacuo to provide 5-bromo-2-(4-fluorophenyl)-N-methyl-6-(methylsulfonamido)benzofuran-3-carboxamide (400 mg. 87%).

¹H-NMR (DMSO, 400 MHz) δ 9.55 (br s, 1H), 8.46˜8.48 (m, 1H), 8.12˜8.17 (m, 2H), 7.96 (s, 1H), 7.87 (s, 1H), 7.45˜7.50 (m, 2H), 3.16 (s, 3H), 2.93 (d, J=8.4 Hz, 3H). MS (M+H)⁺: 441.

### Step 3: 5-bromo-2-(4-fluorophenyl)-6-(N-(3-hydroxypropyl)methylsulfonamido)-N-methylbenzofuran-3-carboxamide

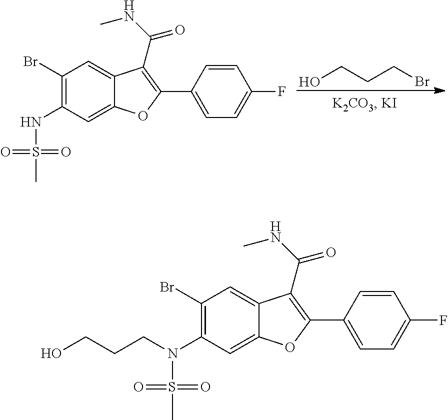

To a solution of 5-bromo-2-(4-fluorophenyl)-N-methyl-6-(methylsulfonamido)benzofuran-3-carboxamide (300 mg, 0.68 mmol) in DMF (10 mL) was added 3-bromopropan-1-ol (190 mg, 1.36 mmol), K₂CO₃ (188 mg, 1.36 mmol) and KI (11 mg, 0.068 mmol). The resulting reaction was heated to 100° C. and allowed to stir at this temperature for 10 hours. The reaction mixture was cooled to room temperature and concentrated in vacuo. The resulting residue was taken up in EtOAc and the resulting solution was washed with H₂O, brine, dried over Na₂SO₄, filtered and concentrated in vacuo. The residue obtained was purified by flash column chromatography (PE:EtOAc=2:1) to provide 5-bromo-2-(4-fluorophenyl)-6-(N-(3-hydroxypropyl)methylsulfonamido)-N-methylbenzofuran-3-carboxamide (320 mg., 78.6%). ¹H-NMR (CDCl₃, 400 MHz) δ 8.12 (s, 1H), 7.76 (d, J=8.0 Hz, 2H), 7.65 (s, 1H), 7.14 (d, J=8.4 Hz, 2H), 5.78 (br s, 1H), 3.64˜3.67 (m, 2H), 3.55˜3.60 (m, 2H), 3.08 (s, 3H), 2.97 (d, J=4.4 Hz, 3H), 1.72˜1.76 (m, 2H). MS (M+H)⁺: 499.

### Step 4: 2-(4-fluorophenyl)-6-(N-(3-hydroxypropyl)methylsulfonamido)-N-methyl-5-(3-(oxazolo[4,5-b]pyridin-2-yl)phenyl)benzofuran-3-carboxamide (Compound 230)

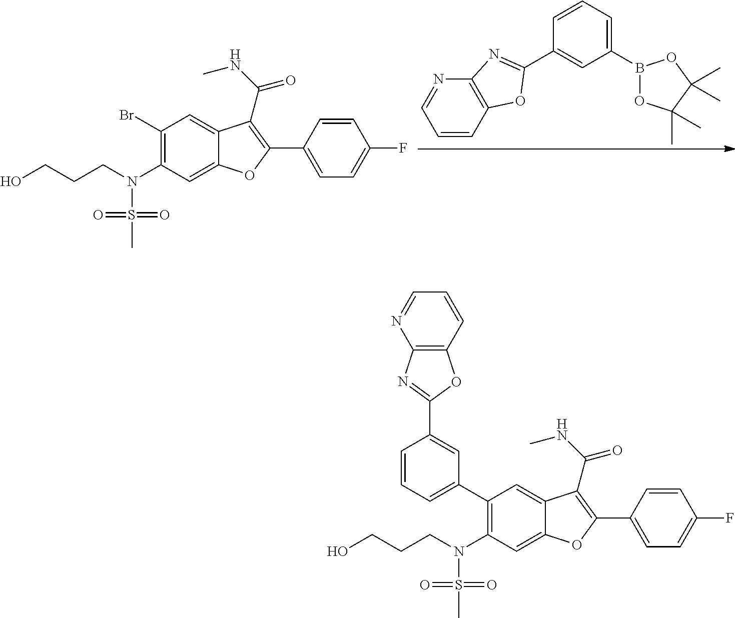

To a degassed solution of 5-bromo-2-(4-fluorophenyl)-6-(N-(3-hydroxypropyl)methylsulfonamido)-N-methylbenzofuran-3-carboxamide (100 mg, 0.20 mmol) and 2-(3-(4,4,5,5-tetramethyl-1,3,2-dioxaborolan-2-yl)phenyl)oxazolo[4,5-b]pyridine (77 mg, 0.24 mmol) in dioxane/CH₃CN/H₂O (10/1/1, 5 mL) was added Pd(PPh₃)₄(2 mg) and K₃CO₃ (100 mg, 0.40 mmol). The reaction was put under N₂ atmosphere and heated to 100° C. in microwave for 30 minutes. The reaction mixture was filtered, and the filtrate was diluted with EtOAc, and the resulting solution washed with H₂O, brine, dried over Na₂SO₄, filtered and concentrated in vacuo. The resulting residue was purified using flash column chromatography (PE:EtOAc=1:1) 2-(4-fluorophenyl)-6-(N-(3-hydroxypropyl)methylsulfonamido)-N-methyl-5-(3-(oxazolo[4,5-b]pyridin-2-yl)phenyl)benzofuran-3-carboxamide (38 mg, 30.9%). ¹H-NMR (CDCl₃, 400 MHz) δ 8.50 (J=4.4 Hz, 1H), 8.38˜8.41 (m, 1H), 8.23 (d, J=8.0 Hz, 1H), 7.81˜7.87 (m, 2H), 7.56˜7.58 (m, 3H), 7.25˜7.26 (m, 2H), 7.18˜7.20 (m, 1H), 7.11˜7.15 (m, 2H), 6.07 (br s, 1H), 3.64˜3.67 (m, 2H), 3.41˜3.52 (m, 2H), 2.92˜2.93 (m, 3H), 2.81 (s, 3H), 1.72˜1.76 (m, 2H). MS (M+H)⁺: 615.

The following compounds of the present invention were prepared using the method described in Example 384 and substituting the appropriate reactants and/or reagents.

| | | | MS |
|---|---|---|---|
| Compound | Structure | NMR | (M + H)⁺ |
| 385 | 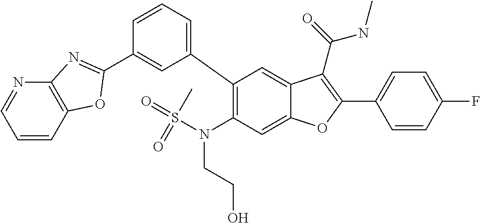 | ¹H-NMR (CDCl₃, 400 MHz) δ 8.57 (d, J = 4.8 Hz, 1H), 8.46 (s, 1H), 8.23 (d, J = 8.0 Hz, 1H), 7.98 (d, J = 8.0 Hz, 1H), 7.81~7.88 (m, 4H), 7.58~7.62 (m, 2H), 7.36~7.41 (m, 1H), 7.12~7.17 (m, 2H), 5.98 (br s, 1H), 3.60~3.70 (m., 3H), 3.38~3.44 (m, 1H), 2.93 (d, J = 4.4 Hz, 3H), 2.89 (s, 3H). | 601 |
| 386 | 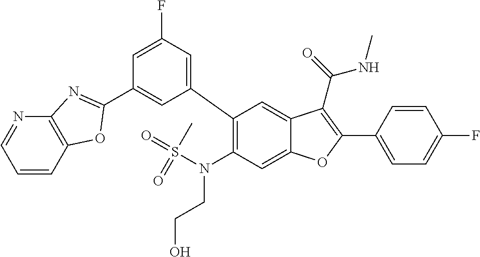 | ¹H-NMR (CDCl₃, 400 MHz) δ 8.64 (s, 1H), 8.36 (s, 1H), 7.92~8.03 (m, 5H), 7.73 (d, J = 4.0 Hz, 1H), 7.64 (d, J = 8.8 Hz, 1H), 7.38~7.42 (m, 1H), 7.23~7.25 (m, 2H), 5.96 (br s, 1H), 3.74~3.87 (m, 3H), 3.47~3.51 (m, 1H), 3.04 (d, J = 4.8 Hz, 3H), 3.03 (s, 3H). | 619 |
| 387 | 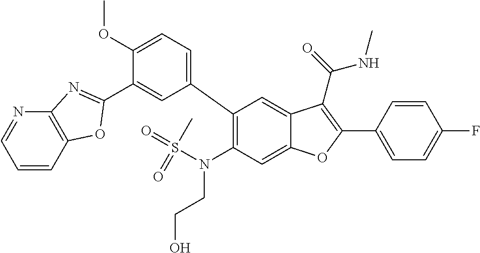 | ¹H-NMR (CDCl₃, 400 MHz) δ 8.53 (d, J = 4.0 Hz, 1H), 8.38 (d, J = 4.0 Hz, 1H), 8.13~8.15 (m, 1H), 7.98~8.00 (m, 2H), 7.94 (d, J = 4.0 Hz, 1H), 7.86 (s, 1H), 7.73 (s, 1H), 7.37~7.49 (m, 1H), 7.30~7.35 (m, 1H), 7.26~7.30 (m, 2H), 4.08 (s, 3H), 3.71~3.74 (m, 1H), 3.46~3.49 (m, 2H), 3.23 (m, 3H), 3.09~3.14 (m, 1H), 2.95 (s, 3H). | 631 |
| 388 | 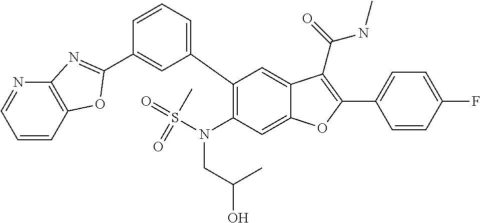 | ¹H-NMR (CDCl₃, 400 MHz) δ 8.49~8.50 (m, 1H), 8.38~8.41 (m, 1H), 8.23~8.24 (m, 1H), 7.81~7.87 (m, 2H), 7.56~7.58 (m, 3H), 7.25~7.26 (m, 2H), 7.18~7.20 (m, 1H), 7.11~7.15 (m, 2H), 5.98 (s, 1H), 3.84~3.85 (m, 1H), 3.53~3.60 (m, 2H), 2.94~3.19 (m, 6H), 1.07~1.12 (m, 3H). | 615 |
| 389 | 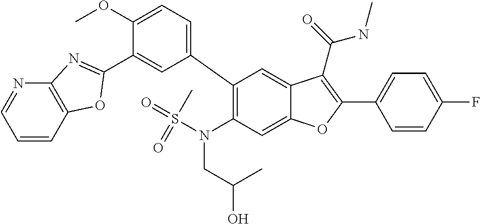 | ¹H-NMR (CDCl₃, 400 MHz) δ 8.58 (d, J = 4.4 Hz, 1H), 8.36 (d, J = 2.0 Hz, 1H), 7.94~7.97 (t, J = 8.0 Hz, 1H), 7.79~7.86 (m, 4H), 7.62 (s, 1H), 7.33~7.35 (m, 2H), 7.12~7.15 (m, 2H), 5.91 (br s, 1H), 4.02 (s, 3H), 3.71~3.76 (m, 1H), 3.43~3.50 (m, 2H), 2.88~2.94 (m, 6H), 0.97~1.07 (m, 3H). | 645 |
| 390 | 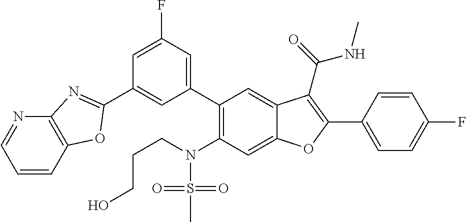 | ¹H-NMR (CDCl₃, 400 MHz) δ 8.55 (d, J = 4.0 Hz, 1H), 8.53 (s, 1H), 8.18~8.20 (m, 1H), 8.08 (d, J = 8.0 Hz, 1H), 8.00~8.03 (m, 2H), 7.93 (s, 1H), 7.78 (s, 1H), 7.71 (d, J = 12.0 Hz, 1H), 7.50~7.53 (m, 1H), 7.27~7.32 (m, 2H), 3.55~3.59 (m, 2H), 3.15 (s, 3H), 2.95 (s, 3H), 1.30~1.60 (m, 4H). | 633 |
| 391 | 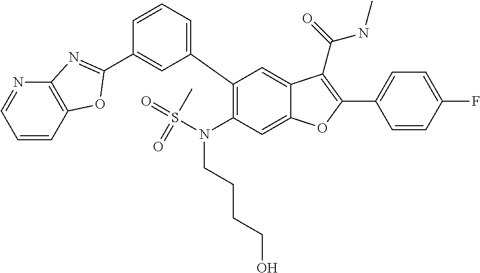 | ¹H-NMR (CDCl₃, 400 MHz) δ 8.50 (d, J = 4.0 Hz, 1H), 8.35 (s, 1H), 8.30 (d, J = 8.0 Hz, 1H), 7.86~7.90 (m, 5H), 7.70~7.72 (t, J = 8.0 Hz, 2H), 7.23~7.26 (t, J = 7.6 Hz, 1H), 7.11~7.15 (m, 2H), 6.03 (br s, 1H), 3.32~3.51 (m, 4H), 2.92 (d, J = 4.8 Hz, 3H), 2.75 (s, 3H), 1.30~1.56 (m, 4H). | 629 |
| 392 | 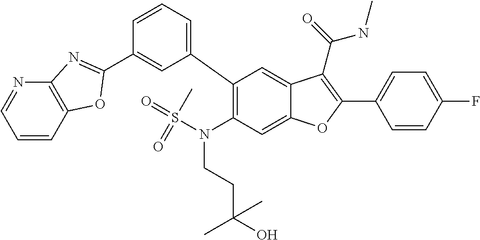 | ¹H-NMR (CDCl₃, 400 MHz) δ 8.50 (d, J = 4.4 Hz, 1H), 8.38~8.41 (m, 1H), 8.23 (d, J = 8.0 Hz, 1H), 7.81~7.87 (m, 2H), 7.56~7.58 (m, 3H), 7.25~7.26 (m, 2H), 7.18~7.20 (m, 1H), 7.11~7.15 (m, 2H), 5.87 (br s, 1H), 3.52~3.62 (m, 2H), 2.93 (d, J = 4.8 Hz, 3H), 2.74 (s, 3H) 1.65~1.67 (m, 2H), 1.07~1.12 (m, 6H). | 643 |
| 393 | 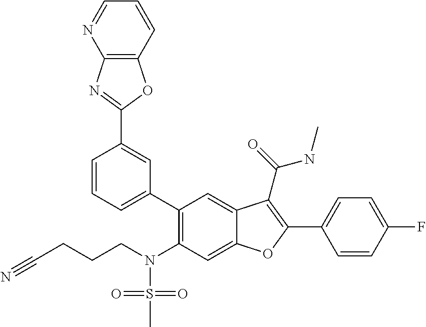 | ¹H-NMR (CDCl₃, 400 MHz) δ 8.74~8.75 (m, 1H), 8.52 (s, 1H), 8.41~8.42 (m, 1H), 8.18~8.20 (m, 1H), 7.95~7.98 (m, 4H), 7.73~7.77 (m, 1H), 7.69 (s, 1H), 7.56~7.59 (m, 1H), 7.24~7.28 (m, 2H), 3.57 (s, 2H), 3.05~3.08 (m, 6H), 2.06~2.10 (m, 2H), 1.75~1.80 (m, 2H). | 624 |
| 394 | 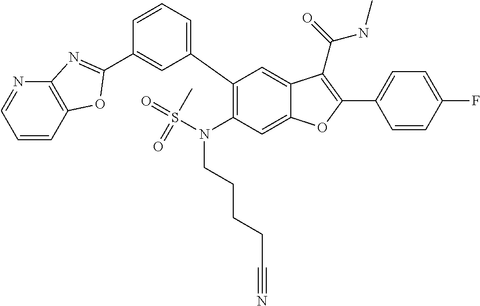 | ¹H-NMR (CDCl₃, 400 MHz) δ 8.72~8.73 (m, 1H), 8.51 (s, 1H), 8.36~8.38 (m, 1H), 8.20~8.22 (m, 1H), 7.90~7.95 (m, 4H), 7.69~7.73 (m, 1H), 7.67 (s, 1H), 7.56~7.58 (m, 1H), 7.22~7.30 (m, 2H), 3.41~3.49 (m, 2H), 3.02~3.05 (m, 6H), 2.22~2.25 (m, 2H), 1.39~1.58 (m, 4H). | 638 |
| 395 | 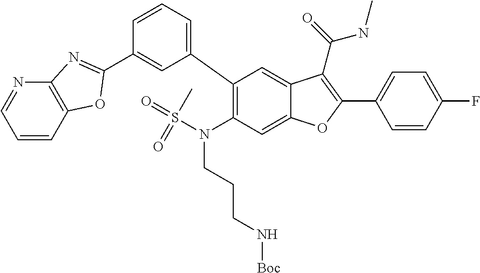 | ¹H-NMR (CDCl₃, 400 MHz) δ 8.67 (d, J = 4.4 Hz, 1H), 8.47 (s, 1H), 8.38 (d, J = 8.0 Hz, 1H), 8.03 (d, J = 8.0 Hz, 1H), 7.96~8.00 (m, 2H), 7.92 (s, 1H), 7.89 (d, J = 7.6 Hz, 1H), 7.70 (d, J = 8.0 Hz, 1H), 7.66 (s, 1H), 7.42~7.46 (m, 1H), 7.23~7.31 (m, 2H), 6.10 (br s, 1H), 4.75 (br s, 1H), 3.43~3.49 (m, 2H), 3.04 (d, J = 4.8 Hz, 3H), 3.02 (s, 3H), 2.84~2.96 (m, 2H), 1.57~1.64 (m, 2H), 1.38 (s, 9H). | 714 |
| 396 | 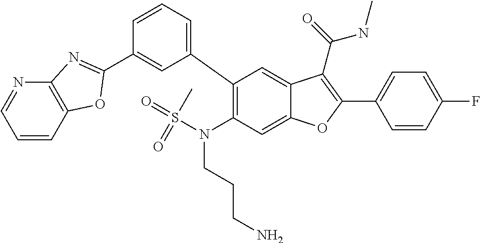 | ¹H-NMR (CDCl₃, 400 MHz) δ 8.39~8.42 (m, 2H), 8.09~8.16 (m, 2H), 7.95~8.01 (m, 2H), 7.82~7.85 (m, 2H), 7.62 (t, J = 8.0 Hz, 1H), 7.57 (s, 1H), 7.38~7.40 (m, 1H), 7.20~7.25 (m, 2H), 6.44 (br s, 1H), 3.50~3.70 (m, 2H), 3.01 (d, J = 4.8 Hz, 3H), 2.97 (s, 3H), 2.80~2.90 (m, 2H), 1.85~1.95 (m, 2H). | 614 |

### Example 397

### 2-(4-fluorophenyl)-N-methyl-6-(N-(2-morpholinoethyl)methylsulfonamido)-5-(3-(oxazolo[4,5-b]pyridin-2-yl)phenyl)benzofuran-3-carboxamide

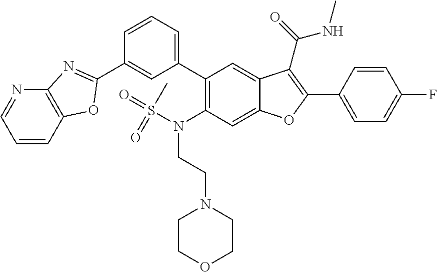

### Step 1: 5-bromo-2-(4-fluorophenyl)-N-methyl-6-(N-(2-morpholinoethyl)methylsulfonamido)benzofuran-3-carboxamide

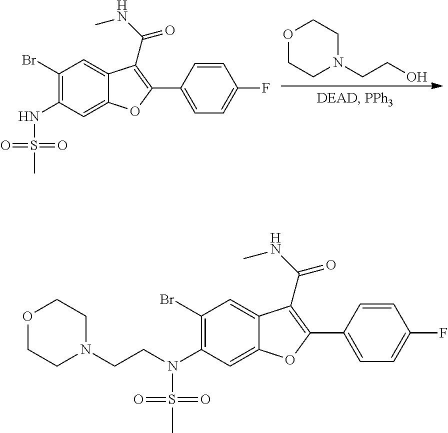

Triphenylphosphine (180 mg, 0.69 mmol) and 5-bromo-2-(4-fluorophenyl)-N-methyl-6-(methylsulfonamido)benzofuran-3-carboxamide (200 mg, 0.45 mmol, prepared by taking the product of Example 1, Step 8 and subjecting it to the methods described in Example 1, Steps 10 and 11) were taken up in anhydrous THF (10 mL) and to the resulting suspension was added DEAD (120 mg, 0.69 mmol). The resulting reaction was allowed to stir at room temperature in the dark for 1 hour, then a solution of 2-morpholinoethanol (90 mg, 0.69 mmol) in anhydrous THF was added, and the resulting reaction was allowed to stir in the dark at room temperature for about 15 hours. The reaction mixture was concentrated in vacuo and the resulting residue was purified using flash chromatography (PE:EtOAc=1:1) to provide 5-bromo-2-(4-fluorophenyl)-N-methyl-6-(N-(2-morpholinoethyl)methylsulfonamido)benzofuran-3-carboxamide (200 mg, 79%). ¹H-NMR (CDCl₃, 400 MHz) δ 8.15 (s, 1H), 7.87˜7.91 (m, 2H), 7.73 (s, 1H), 7.18˜7.23 (m, 2H), 5.93 (br s, 1H), 4.04˜4.12 (m, 1H), 3.59˜3.66 (m, 5H), 3.11 (s, 3H), 2.99 (d, J=4.4 Hz, 3H), 2.48˜2.55 (m, 4H), 2.33˜2.37 (m, 2H). MS (M+H)⁺: 554.

### Step 2: 2-(4-fluorophenyl-N-methyl-6-(N-(2-morpholinoethyl)methylsulfonamido)-5-(3-(oxazolo[4,5-b]pyridine-2-yl)phenyl)benzofuran-3-carboxamide

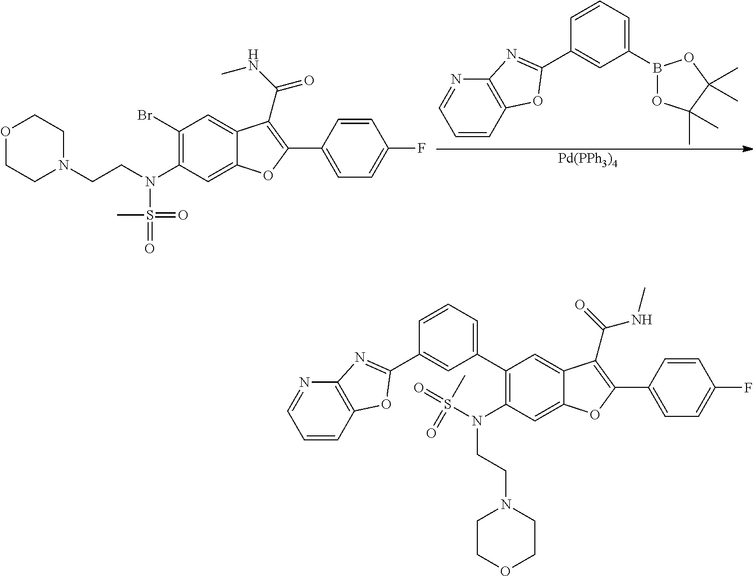

5-bromo-2-(4-fluorophenyl)-N-methyl-6-(N-(2-morpholinoethyl)methylsulfonamido)benzofuran-3-carboxamide (20 mg, 0.04 mmol), 2-(3-(4,4,5,5-tetramethyl-1,3,2-dioxaborolan-2-yl)phenyl)oxazolo[4,5-b]pyridine (12 mg, 0.04 mmol) and K₂CO₃ (10 mg, 0.07 mmol) were taken up in a mixture of dioxane/CH₃CN/H₂O (10/1/1, 1 mL total solution volume). To the resulting solution was added Pd(PPh₃)₄ (2 mg) and the resulting reaction was put under N₂ atmosphere and heated to 100° C. using microwave radiation. The reaction was allowed to remain at this temperature under microwave radiation for 20 minutes, then was cooled to room temperature and concentrated in vacuo. The residue obtained was purified using preparative HPLC to provide 2-(4-fluorophenyl)-N-methyl-6-(N-(2-morpholinoethyl)methylsulfonamido)-5-(3-(oxazolo[4,5-b]pyridin-2-yl)phenyl)benzofuran-3-carboxamide (15 mg, 62%). ¹H-NMR (CDCl₃, 400 MHz) δ 8.56 (br s, 1H), 8.30 (s, 1H), 8.20˜8.22 (m, 1H), 7.97 (d, J=8.0 Hz, 1H), 7.81˜7.87 (m, 3H), 7.71 (br s, 1H), 7.58˜7.63 (m, 2H), 7.36˜7.40 (m, 1H), 7.14˜7.19 (m, 2H), 6.37 (br s, 1H), 3.80˜4.05 (m, 6H), 3.42 (br s, 2H), 3.21 (br s, 2H), 2.80˜3.10 (m, 8H). MS (M+H)⁺: 670.

The following compounds of the present invention were prepared using the method described in Example 397 and substituting the appropriate reactants and/or reagents.

| | | | MS |
|---|---|---|---|
| Compound | Structure | NMR | (M + H)⁺ |
| 398 | 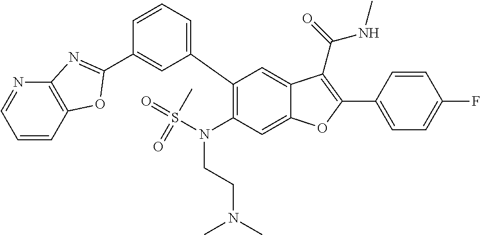 | ¹H-NMR (CDCl₃, 400 MHz) δ 8.48~8.53 (m, 2H), 8.35 (d, J = 8.0 Hz, 1H), 8.15 (d, J = 8.0 Hz, 1H), 7.92~7.99 (m, 4H), 7.75~7.87 (m, 2H), 7.46~7.49 (m, 1H), 7.26~7.30 (m, 2H), 3.89~3.94 (m, 2H), 3.36~3.40 (m, 1H), 3.20~3.22 (m, 1H ), 3.06 (s, 3H), 2.93 (d, J = 4.0 Hz, 3H), 2.81 (s, 6H). | 628 |
| 399 | 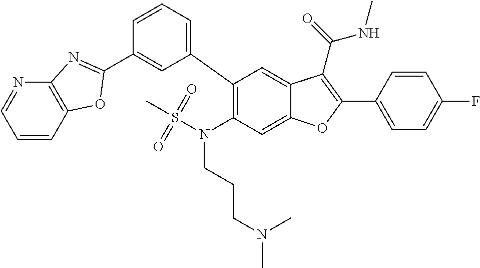 | ¹H-NMR (CDCl₃, 400 MHz) δ 8.64 (d, J = 4.8 Hz, 1H), 8.42 (s, 1H), 8.31 (d, J = 8.0 Hz, 1H), 8.13 (d, J = 8.4 Hz, 1H), 7.88~7.95 (m, 4H), 7.65 (t, J = 8.0 Hz, 1H), 7.63 (s, 1H), 7.50~7.54 (m, 1H), 7.23 (t, J = 8.8 Hz, 2H), 6.12 (d, J = 4.8 Hz, 1H), 3.60~3.75 (m, 2H), 2.95~3.04 (m, 7H), 2.78~2.87 (m, 7H), 1.98~2.05 (m, 2H). | 642 |

### Example 400

### 2-(4-fluorophenyl)-N-methyl-6-(N-methylphenylsulfonamido)-5(3-(oxazolo[4,5-b]pyridin-2-yl)phenyl)benzofuran-3-carboxamide

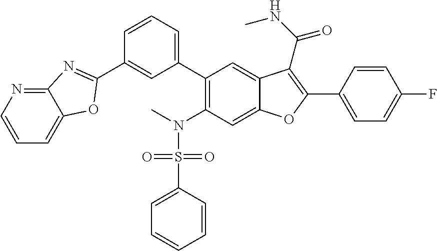

### Step 1: methyl 6-amino-5-bromo-2-(4-fluorophenyl)benzofuran-3-carboxylate

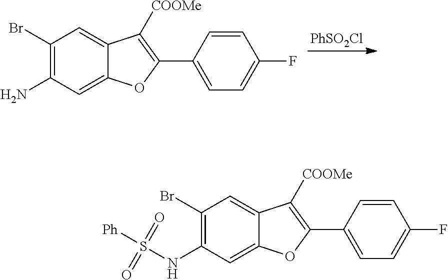

To a 0° C. solution of methyl 6-amino-5-bromo-2-(4-fluorophenyl)benzofuran-3-carboxylate (prepared as described in Example 1, Step 7, 500 mg, 1.4 mmol) and pyridine (5 mL) in dry dichloromethane (10 mL) was added benzenesulfonyl chloride (1.5 g, 8.5 mmol). The cold bath was removed and the resulting reaction was allowed to stir for about 15 hours at room temperature. The reaction mixture was diluted with water, extracted with dichloromethane and the organic extract was washed with brine, dried (Na₂SO₄), filtered and concentrated in vacuo. The residue obtained was purified using flash column chromatography (PE:EtOAc=5:1) to provide methyl 5-bromo-2-(4-fluorophenyl)-6-(phenylsulfonamido)benzofuran-3-carboxylate (600 mg, 87%). ¹H-NMR (CDCl₃, 400 MHz) δ 8.01˜8.03 (m, 2H), 7.93˜7.95 (d, 2H), 7.68˜7.69 (d, 1H), 7.62˜7.63 (m, 1H), 7.50˜7.52 (m, 2H), 7.33˜7.37 (m, 1H) 7.10˜7.16 (m, 2H) 5.23 (s, 1H). 3.85˜3.89 (d, J=16.8 Hz, 3H). MS (M+H)⁺: 504.

### Step 2: methyl 5-bromo-2-(4-fluorophenyl)-6-(N-methylphenylsulfonamido)benzofuran-3-carboxylate

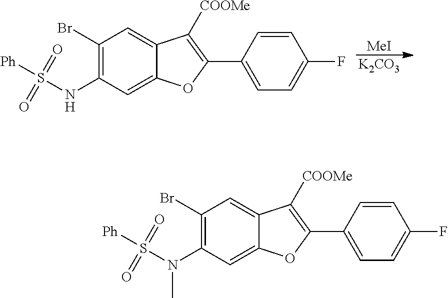

A solution of methyl 5-bromo-2-(4-fluorophenyl)-6-(phenylsulfonamido)benzofuran-3-carboxylate (0.6 g, 1.18 mmol) and K₂CO₃ (1.1 g, 8.0 mmol) in DMF (15 mL) was put under N₂ atmosphere. CH₃I (1.0 mL, 16.0 mmol) was added and the resulting reaction was heated to 40° C. and allowed to stir at this temperature for about 15 hours. The reaction mixture was then filtered and the filtrate was concentrated in vacuo to provide methyl 5-bromo-2-(4-fluorophenyl)-6-(N-methylphenylsulfonamido)benzofuran-3-carboxylate (500 mg, 81%) which was used without further purification.

### Step 3: 5-bromo-2-(4-fluorophenyl)-6-(N-methylphenylsulfonamido)benzofuran-3-carboxylic acid

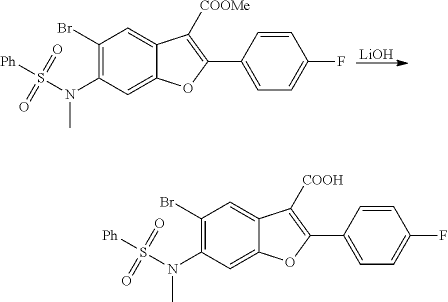

To a solution of methyl 5-bromo-2-(4-fluorophenyl)-6-(N-methylphenylsulfonamido)benzofuran-3-carboxylate (500 mg, 0.96 mmol) in a mixture of dioxane/H₂O (1/1, 10 mL total volume) was added LiOH.H₂O (90 mg, 2.14 mmol), and the resulting reaction was heated to 100° C. and allowed to stir at this temperature for 2 hours. The reaction mixture was cooled to room temperature, then concentrated in vacuo. The residue obtained was dissolved in H₂O and the resulting solution was adjusted to pH 3 using HCl (1 N). The acidific solution was then extracted with EtOAc and the organic extract was washed with brine, dried over Na₂SO₄, filtered and concentrated in vacuo to provide 5-bromo-2-(4-fluorophenyl)-6-(N-methylphenylsulfonamido)benzofuran-3-carboxylic acid (300 mg, 62%), which was used without further purification.

### Step 4: 5-bromo-2-(4-fluorophenyl)-N-methyl-6-(N-methylphenylsulfonamido)benzofuran-3-carboxamide

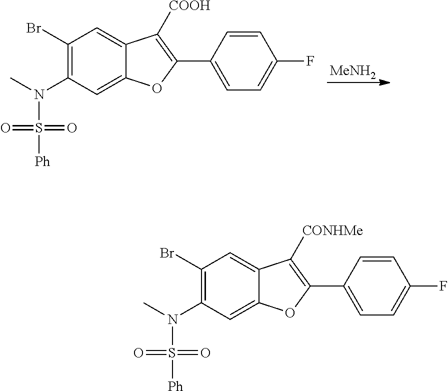

To a solution of 5-bromo-2-(4-fluorophenyl)-6-(N-methylphenylsulfonamido)benzofuran-3-carboxylic acid (300 mg, 0.59 mmol) in dry DMF (10 mL) was added HOBT (100 mg, 0.74 mmol) and EDCI (100 mg, 0.64 mmol) and the resulting reaction was allowed to stir at room temperature for 1 hour. Et₃N (2.0 mL) and CH₃NH₂ (HCl salt, 100 mg, 1.48 mmol) were then added to the reaction mixture and the resulting reaction was allowed to stir for about 15 hours at room temperature. The reaction mixture was concentrated in vacuo, the resulting residue was diluted with H₂O, and the resulting aqueous solution was extracted with ethyl acetate. The organic extract was washed with H₂O and brine, then concentrated in vacuo. The residue obtained was purified by flash column chromatography (PE:EtOAc=2:1) to provide 5-bromo-2-(4-fluorophenyl)-N-methyl-6-(N-methylphenylsulfonamido)benzofuran-3-carboxamide (130 mg, 42%). ¹H-NMR (CDCl₃, 400 MHz) δ 8.02 (s, 1H), 7.83˜7.86 (m, 2H), 7.75˜7.77 (d, 2H), 7.54˜7.56 (m, 1H), 7.44˜7.48 (m, 2H), 7.36 (s, 1H), 7.11˜7.19 (m, 2H), 5.71 (br s, 1H), 3.20 (s, 3H), 2.94 (d, J=4.8 Hz, 3H). MS (M+H)⁺: 517.

### Step 5: 2-(4-fluorophenyl)-N-methyl-6-(N-methylphenylsulfonamido)-5-(3-(oxazolo[4,5-b]pyridin-2-yl)phenyl)benzofuran-3-carboxamide (Compound 246)

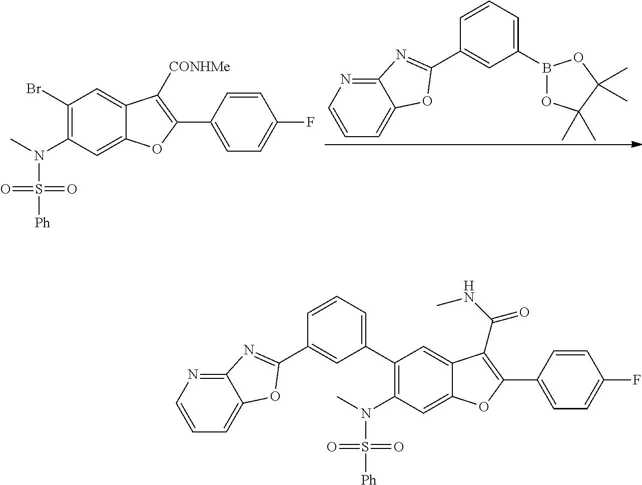

5-bromo-2-(4-fluorophenyl)-N-methyl-6-(N-methylphenylsulfonamido)benzofuran-3-carboxamide (30 mg, 0.06 mmol), 2-(3-(4,4,5,5-tetramethyl-1,3,2-dioxaborolan-2-yl)phenyl)oxazolo[4,5-b]pyridine (22.5 mg, 0.07 mmol) and K₂CO₃ (16 mg, 0.12 mmol) were taken up in a mixture of dioxane-acetonitrile-water (10:1:1, 2 mL total volume). To the resulting solution was added Pd(PPh₃)₄ (5 mg) and the resulting reaction was put under N₂ atmosphere and heated to 100° C. using microwave radiation. The reaction was allowed to remain at this temperature under microwave radiation for 20 minutes, then was cooled to room temperature and concentrated in vacuo. The residue obtained was purified using preparative HPLC to provide 2-(4-fluorophenyl)-N-methyl-6-(N-methylphenylsulfonamido)-5-(3-(oxazolo[4,5-b]pyridin-2-yl)phenyl)benzofuran-3-carboxamide (4 mg, 11%). ¹H-NMR (CDCl₃, 400 MHz) δ 8.57 (d, 1H), 8.30 (m, 2H), 7.86˜7.90 (m, 3H), 7.82 (s, 1H), 7.68 (d, 1H), 7.53˜7.58 (m, 3H), 7.47˜7.49 (m, 1H), 7.36˜7.40 (m, 2H), 7.30˜7.33 (m, 1H), 7.12˜7.15 (m, 3H), 5.83 (br s, 1H), 3.06 (s, 3H), (d, J=4.8 Hz, 3H). MS (M+H)⁺: 633.

The following compound of the present invention was prepared using the method described in Example 400 and substituting the appropriate reactants and/or reagents.

| | | | MS |
|---|---|---|---|
| Compound | Structure | NMR | (M + H)⁺ |
| 401 | 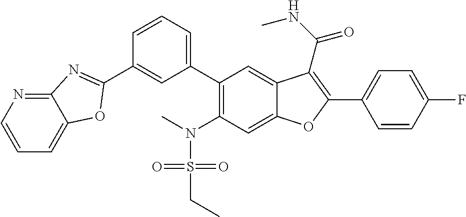 | ¹H-NMR (CDCl₃, 400 MHz) δ 8.57~8.58 (d, J = 4.0 Hz, 1H), 8.36 (s, 1H), 8.29~8.31 (d, J = 8.2 Hz, 1H), 7.82~7.98 (m, 4H), 7.57~7.60 (m, 3H), 7.27~7.29 (m, 1H), 7.13~7.17 (m, 2H), 5.82~5.83 (d, J = 8.1 Hz, 1H), 3.15 (s, 3H), 2.93~2.94 (d, J = 5.2 Hz, 3H), 2.76~2.78 (m, 2H), 1.09~1.13 (m, 3H). | 585 |

### Example 402

### 5-(3-(4-fluorobenzo[d]oxazol-2-yl)-4-methoxyphenyl)-2-(4-fluorophenyl)-N-methyl-6-(N-methylmethylsulfonamido)benzofuran-3-carboxamide

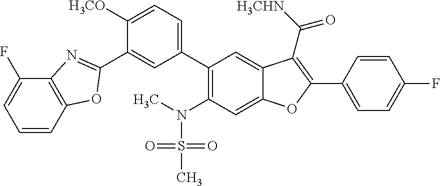

### Step 1: 1-fluoro-3-methoxy-2-nitrobenzene

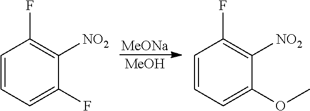

To a 0° C. solution of 1,3-difluoro-2-nitrobenzene (100 g, 0.63 mol) in MeOH (1.3 L) was slowly added a solution of MeONa (0.69 mol, in MeOH, freshly prepared from 15.9 g of sodium metal and 200 mL of MeOH). The resulting reaction was allowed to stir for about 15 hours at room temperature, then the reaction mixture was concentrated and diluted with EtOAc. The organic phase was washed sequentially with water and brine, dried over Na₂SO₄, then filtered and concentrated in vacuo to provide 1-fluoro-3-methoxy-2-nitrobenzene (98 g, yield: 91.4%), which was used without further purification. ¹H-NMR (CDCl₃, 400 MHz) δ 7.38˜7.44 (m, 1H), 6.72˜6.88 (m, 2H), 3.95 (s, 3H).

### Step 2: 3-fluoro-2-nitrophenol

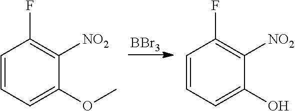

To a −40° C. solution of 1-fluoro-3-methoxy-2-nitrobenzene (98 g, 0.57 mol) in dichloromethane (500 mL) was added dropwise a solution of BBr₃ (1 L, 1 M in dichloromethane The resulting reaction was allowed to stir for about 15 hours at room temperature, then the reaction mixture was slowly poured into ice water (500 mL). The resulting solution was extracted with EtOAc (300 mL×3), and the combined organic layers were washed with sequentially with 5% aqueous NaHCO₃ and brine, then dried over Na₂SO₄, filtered and concentrated in vacuo to provide 3-fluoro-2-nitrophenol (85 g, yield: 95%), which was used without further purification. ¹H-NMR (CDCl₃, 400 MHz) δ 7.43˜7.49 (m, 1H), 6.88 (d, J=8.0 Hz, 1H), 6.73˜6.78 (m, 1H).

### Step 3: 5-(3-(4-fluorobenzo[d]oxazol-2-yl)-4-methoxyphenyl)-2-(4-fluorophenyl)-N-methyl-6-(N-methylsulfonamido)benzofuran-3-carboxamide

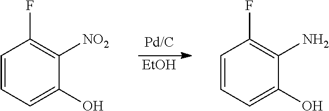

3-fluoro-2-nitrophenol (38 g, 0.24 mol) was dissolved in EtOH and then palladium on carbon (5 g, 10% Pd) was added. The reaction flask was evacuated and the reaction mixture was put under H₂ atmosphere (1 atm) and allowed to stir for 3 hours at room temperature. The reaction mixture was then filtered through a short pad of celite and the celite was washed with EtOH. The combined filtrate and washing was concentrated in vacuo to provide 2-amino-3-fluorophenol (26 g, yield: 85.7%), which was used without further purification. ¹H-NMR (DMSO, 400 MHz) δ 9.43 (s, 1H), 6.42˜6.53 (m, 2H), 6.32˜6.42 (m, 1H), 4.34 (s, 2H).

### Step 4: 2-(5-bromo-2-methoxyphenyl)-4-fluorobenzo[d]oxazole

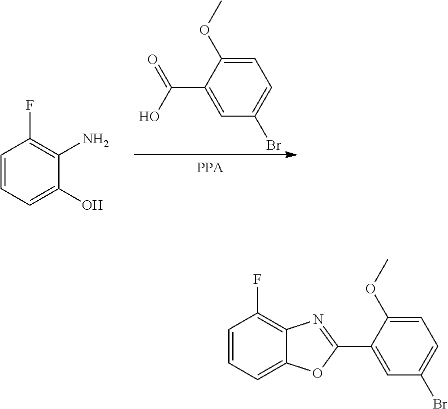

To a solution of 2-amino-3-fluorophenol (9 g 70.8 mmol) in 10 mL of PPA was added 5-bromo-2-methoxybenzoic acid (16.3 g, 70.8 mmol), and the resulting reaction was heated to 140° C. and allowed to stir at this temperature for 4 hours. The reaction mixture was then poured into ice water (50 mL), and extracted with EtOAc. The organic extract was concentrated in vacuo and the residue obtained was purified using flash column chromatography on silica gel (petroleum ether/ethyl acetate=10/1), to provide 2-(5-bromo-2-methoxyphenyl)-4-fluorobenzo[d]oxazole (16 g, yield: 82%) as a solid. ¹H-NMR (CDCl₃, 400 MHz) δ 8.29 (d, J=2.4 Hz, 1H), 7.57˜7.54 (m, 1H), 7.40 (d, J=8.0 Hz, 1H), 7.27˜7.33 (m, 1H), 7.07 (m, 1H), 6.96 (d, J=9.2 Hz, 1H), 3.99 (s, 3H).

### Step 5: 4-fluoro-2-(2-methoxy-5-(4,4,5,5-tetramethyl-1,3,2-dioxaborolan-2-yl)phenyl)benzo[d]oxazole

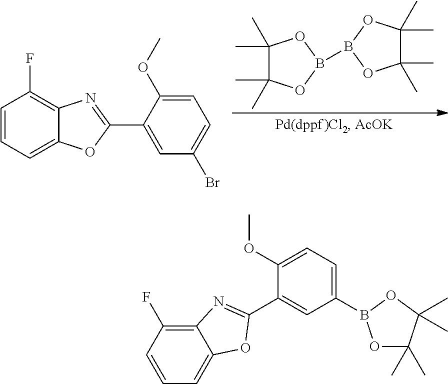

A solution of 2-(5-bromo-2-methoxyphenyl)-4-fluorobenzo[d]oxazole (18.4 g, 57.1 mmol) and bis(pinacolato)diboron (17.4 g, 68.5 mmol) in DMF (10 mL) was placed under N₂ atmosphere and to the resulting solution was added Pd(dppf)Cl₂ (500 mg) and AcOK (10 g, 114 mmol). The reaction was heated to 80° C. and allowed to stir at this temperature for 3 hours. The reaction mixture was then concentrated in vacuo, the residue obtained was dissolved in dichloromethane, and the resulting solution was filtered through a pad of celite. The organic solution was washed sequential with H₂O and brine, then dried over Na₂SO₄, filtered and concentrated in vacuo. The resulting residue was purified using flash column chromatography on silica gel (PE/EA=10/1) to provide 4-fluoro-2-(2-methoxy-5-(4,4,5,5-tetramethyl-1,3,2-dioxaborolan-2-yl)phenyl)benzo[d]oxazole (10 g, yield: 54%) as a solid. ¹H-NMR (CDCl₃, 400 MHz) δ 8.53 (d, J=1.6 Hz, 1H), 7.85˜7.92 (m, 1H), 7.44 (d, J=8.0 Hz, 1H), 7.20˜7.28 (m, 1H), 6.96˜7.05 (m, 2H), 3.97 (s, 3H), 1.29 (s, 12H).

### Step 6—5-(3-(4-fluorobenzo[d]oxazol-2-yl)-4-methoxyphenyl)-2-(4-fluorophenyl)-N-methyl-6-(N-methylsulfonamido)benzofuran-3-carboxamide

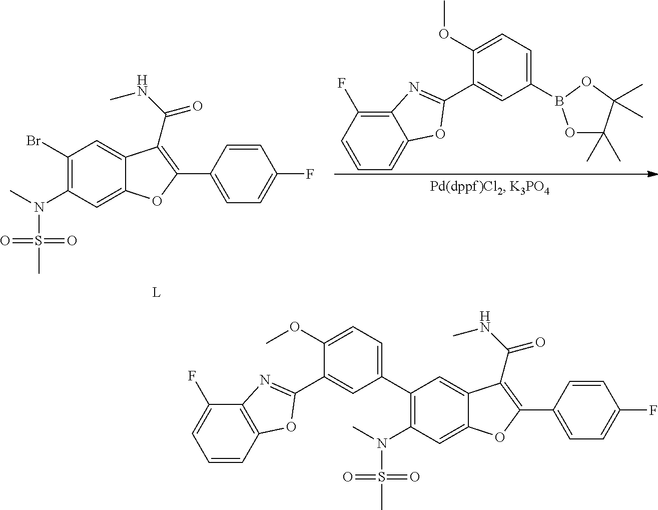

To a solution of Compound L (5 g, 11.0 mmol) and 4-fluoro-2-(2-methoxy-5-(4,4,5,5-tetramethyl-1,3,2-dioxaborolan-2-yl)phenyl)benzo[d]oxazole (5.27 g, 14.3 mmol) in DMF (150 mL) under N₂ atmosphere was added Pd(dppf)Cl₂ (200 mg) and K₃PO₄ (4.66 g, 22.0 mmol). The resulting reaction was heated to 100° C. and allowed to stir at this temperature for 10 hours, then the reaction mixture was concentrated in vacuo. The residue obtained was dissolved in dichloromethane and filtrated through a short pad of celite. The filtrate was washed sequentially with water and brine, dried over Na₂SO₄, then filtered and concentrated in vacuo. The resulting residue was purified using flash column chromatography on silica gel (petroleum ether/ethyl acetate=4/1 to 2/1) and the product obtained was then recrystallized from dichlormethane/ethyl acetate (5/1), to provide 5-(3-(4-fluorobenzo[d]oxazol-2-yl)-4-methoxyphenyl)-2-(4-fluorophenyl)-N-methyl-6-(N-methylsulfonamido)benzofuran-3-carboxamide (3.8 g, yield: 56%) as a white solid. ¹H-NMR (CDCl₃, 400 MHz) δ 8.21 (d, J=2.0 Hz, 1H), 7.91˜7.95 (m, 2H), 7.83 (s, 1H), 7.68 (d, J=2.0 Hz, 1H), 7.66 (s, 1H), 7.39 (d, J=8.0 Hz, 1H), 7.14˜7.27 (m, 4H), 7.06 (t, J=8.4 Hz, 1H), 5.95 (br s, 1H), 4.06 (s, 3H), 3.14 (s, 3H), 2.99 (d, J=4.8 Hz, 3H), 2.77 (s, 3H); MS (M+H)⁺618.

### Example 403

### 5-(3-(4-cyanobenzo[d]oxazol-2-yl)-4-methoxyphenyl)-2-(4-fluorophenyl)-N-methyl-6-(N-methylmethylsulfonamido)benzofuran-3-carboxamide

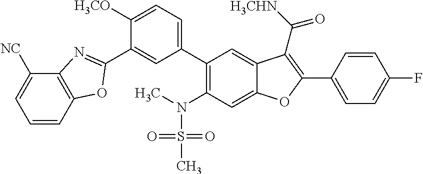

### Step 1: 3-hydroxy-2-nitrobenzonitrile

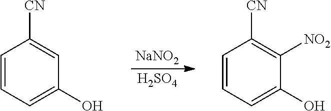

To a 0° C. solution of NaNO₃ (4 g, 47 mmol) and H₂SO₄ (aqueous, 3 M, 45 mL) was added a solution of 3-hydroxybenzonitrile (5 g, 42 mmol) in CH₂Cl₂ (80 mL). To the resulting solution was added NaNO₂ (289 mg, 4.2 mmol) and the resulting reaction was allowed to stir for 16 hours. The reaction mixture was then diluted with CH₂Cl₂ and the resulting solution was washed sequentially with H₂O and brine, filtered and concentrated in vacuo. The residue obtained was purified using flash column chromatography on silica gel (petroleum ether/ethyl acetate=40/1) to provide 3-hydroxy-2-nitrobenzonitrile (1.7 g, yield: 25%). ¹H-NMR (DMSO, 400 MHz) δ 11.73 (s, 1H), 8.25 (d, J=8.4 Hz, 1H), 7.35 (d, J=4.4 Hz, 1H), 7.19 (t, J=8.4 Hz, 1H)

### Step 2: 2-amino-3-hydroxybenzonitrile

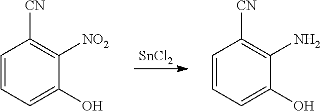

To a solution of 3-hydroxy-2-nitrobenzonitrile (1.7 g, 0.01 mol) in MeOH (30 mL) was added SnCl₂ (7.9 g, 4.1 mol). The resulting reaction was heated to 50° C. and allowed to stir at this temperature for 6 hours. The reaction mixture was then concentrated in vacuo and the resulting residue was taken up in EtOAc. To the resulting solution was added saturated aqueous NaHCO₃ solution, which caused a white solid to precipitate out of solution. The resulting suspension was filtered through celite and extracted with EtOAc. The organic layer was dried over MgSO₄, filtered, and concentrated in vacuo to provide 2-amino-3-hydroxybenzonitrile (1.1 g, yield: 79.7%), which was used without further purification. ¹H-NMR (CDCl₃, 400 MHz) δ 6.94 (d, J=8.4 Hz, 1H), 6.79 (d, J=8.0 Hz, 1H), 6.53 (t, 1=8.0 Hz, 1H), 5.17 (s, 1H), 4.43 (s, 2H).

### Step 3: 5-bromo-N-(2-cyano-6-hydroxyphenyl)-2-methoxybenzamide

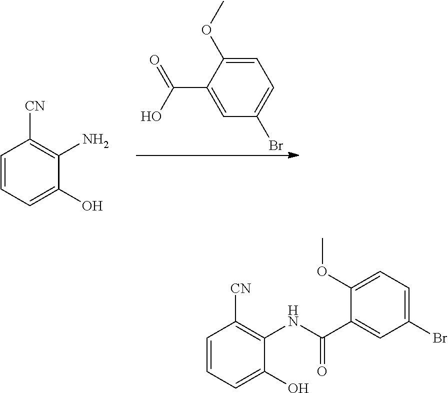

A solution of 5-bromo-2-methoxybenzoic acid (11.7 g, 50.8 mmol) in SOCl₂ (50 mL) was heated to 100° C. and allowed to stir at this temperature for 2 hours. The reaction mixture was then concentrated in vacuo and the resulting residue was dissolved in dry dichloromethane (30 mL). The resulting solution was then added dropwise to a solution of 2-amino-3-hydroxybenzonitrile (6.2 g, 46.22 mmol) in dichloromethane (30 mL) and triethylamine (15 mL) at 0° C. under N₂. The resulting reaction was allowed to stir for 5 hours at room temperature, then the reaction mixture was poured into ice water (50 mL) and extracted with dichloromethane. The organic phase was washed sequentially with H₂O and brine, dried over Na₂SO₄, filtered and concentrated in vacuo to provide 5-bromo-N-(2-cyano-6-hydroxyphenyl)-2-methoxybenzamide (4.0 g), which was used without further purification.

### Step 4: 2-(5-bromo-2-methoxyphenyl)benzo[d]oxazole-4-carbonitrile

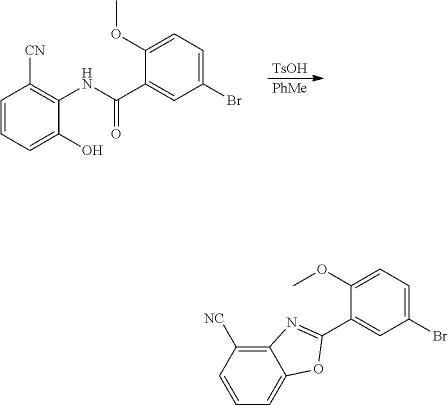

A solution of 5-bromo-N-(2-cyano-6-hydroxyphenyl)-2-heated to reflux and allowed to stir at this temperature for 3 hours using a reflux condenser fitted with a Dean-Stark trap. After the was removed, the residue obtained was dissolved in EtOAc (40 mL). The organic phase was washed sequentially with H₂O and brine, dried over Na₂SO₄, filtered and concentrated in vacuo. The residue obtained was purified using flash column chromatography on silica gel (PE/EA=10/1) to provide 2-(5-bromo-2-methoxyphenyl)benzo[d]oxazole-4-carbonitrile (2.1 g, yield: 26% two steps) as solid. ¹H-NMR (CDCl₃, 400 MHz) δ 8.70 (s, 1H), 8.21˜8.24 (m, 1H), 7.81˜7.83 (m, 1H), 7.70˜7.72 (m, 1H), 7.46˜7.48 (m, 1H), 7.15˜7.17 (m, 1H), 4.14 (s, 3H).

### Step 5: 2-(2-methoxy-5-(4,4,5,5-tetramethyl-1,3,2-dioxaborolan-2-yl)phenyl)benzo[d]oxazole-4-carbonitrile

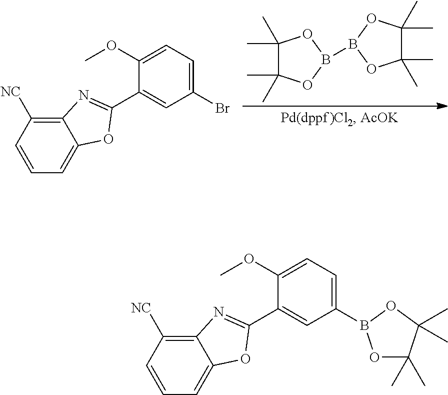

To a solution of 2-(5-bromo-2-methoxyphenyl)benzo[d]oxazole-4-carbonitrile (2.0 g, 6.08 mmol) and bis(pinacolato)diboron (2.01 g, 7.90 mmol) in toluene (25 mL) under N₂ atmosphere, was added Pd(dppf)Cl₂ (300 mg) and AcOK (1.19 g, 12.15 mmol). The resulting reaction was heated to 80° C. and allowed to stir at this temperature for 3 hours. The reaction mixture was then concentrated in vacuo and the resulting residue was dissolved in dichloromethane and filtrated through a short pad of celite. The organic phase was washed sequentially with H₂O and brine, dried over Na₂SO₄, filtered and concentrated in vacuo. The residue obtained was purified using flash column chromatography on silica gel (petroleum ether/ethyl acetate=10/1) to provide 2-(2-methoxy-5-(4,4,5,5-tetramethyl-1,3,2-dioxaborolan-2-yl)phenyl)benzo[d]oxazole-4-carbonitrile (1.8 g, yield: 78.6%) as solid, which was used without further purification. ¹H-NMR (CDCl₃, 400 MHz) δ 8.65 (s, 1H), 8.00˜8.02 (m, 1H), 7.84˜7.86 (m, 1H), 7.68˜7.70 (m, 1H), 7.42˜7.46 (m, 1H), 7.10˜7.12 (m, 1H), 4.08 (s, 3H), 1.41 (s, 12H).

### Step 6: 5-(3-(4-cyanobenzo[d]oxazol-2-yl)-4-methoxyphenyl)-2-(4-fluorophenyl)-N-methyl-6-(N-methylsulfonamido)benzofuran-3-carboxamide

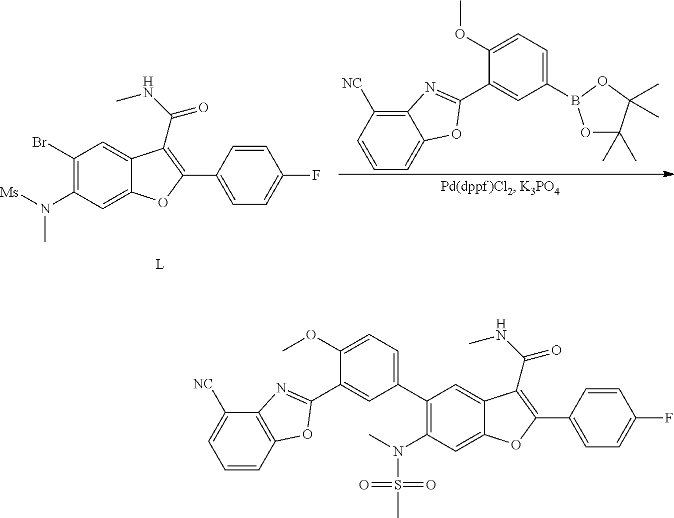

To a solution of Compound L (1.21 g, 2.66 mmol) and 2-(2-methoxy-5-(4,4,5,5-tetramethyl-1,3,2-dioxaborolan-2-yl)phenyl)benzo[d]oxazole-4-carbonitrile (1.20 g, 3.19 mmol) in DMF (12 mL) under N₂ atmosphere, was added Pd(dppf)Cl₂ (400 mg) and K₃PO₄ (1.42 g, 5.32 mmol). The resulting reaction was heated to 100° C. and allowed to stir at this temperature for 10 hours, then the reaction mixture was cooled to room temperature and concentrated in vacuo. The residue obtained was dissolved in dichloromethane and filtered through a short pad of celite. The filtrate was washed sequentially with water and brine, dried over Na₂SO₄, filtered and concentrated in vacuo. The resulting residue was purified using preparative HPLC to provide the title compound (0.81 g, yield: 50%) as white solid. ¹H-NMR (CDCl₃, 400 MHz) δ 8.25 (s, 1H), 7.86˜7.89 (m, 2H), 7.76˜7.80 (m, 2H), 7.59˜7.67 (m, 3H), 7.34˜7.38 (m, 1H), 7.11˜7.16 (m, 3H), 5.85 (s, 1H), 4.02 (s, 3H), 3.10 (s, 3H), 2.93 (d, J=4.8 Hz, 3H), 2.78 (s, 3H);

MS (M+H)⁺625.

### Example 411

### 5-[3-(4-Fluoro-benzooxazol-2-yl)-4-methoxy-phenyl]-2-(4-fluoro-phenyl)-6-(methanesulfonyl-methyl-amino)-benzofuran-3-carboxylic acid methylamide

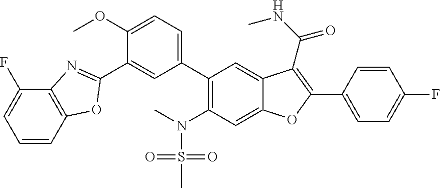

### Step 1-Synthesis of ethyl 5-bromo-2-(4-fluorophenyl)benzofuran-3-carboxylate

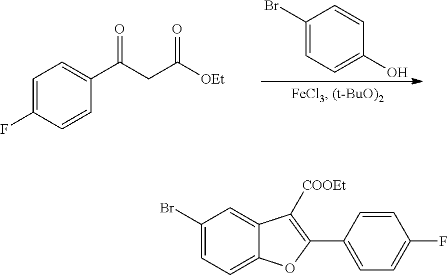

A solution of ethyl 3-(4-fluorophenyl)-3-oxopropanoate (130 g, 0.6 mol), 4-bromophenol (311 g, 1.8 mol) and FeCl₃.6H₂O (19.5 g, 0.09 mol) in DCE (700 mL) was heated to reflux, and then 2-(tert-butylperoxy)-2-methylpropane (193 g, 1.32 mol) was added drop wise under nitrogen. After 6 hours of refluxing, the mixture was cooled to room temperature and quenched with saturated NaHSO₃, extracted with dichloromethane. The organic phases were washed with water, brine and dried over Na₂SO₄, filtered and concentrated in vacuo. The residue obtained was purified using column chromatography (petroleum ether:dichloromethane=15:1) to provide the crude product, which was crystallized from cold MeOH to provide ethyl 5-bromo-2-(4-fluorophenyl)benzofuran-3-carboxylate (37 g, 14.3%) as a solid. ¹H-NMR (CDCl₃, 400 MHz) δ 8.12 (s, 1H), 7.97˜8.01 (m, 2H), 7.37 (d, J=4.0 Hz, 1H), 7.32 (d, J=8.0 Hz, 1H), 7.11 (t, J=8.0 Hz, 2H), 4.32-4.38 (m, 2H), 1.36 (t, J=8.0 Hz, 3H). MS (M+H)⁺: 363/365.

### Step 2—Synthesis of ethyl-5-bromo-2-(4-fluorophenyl)-6-nitrobenzofuran-3-carboxylate

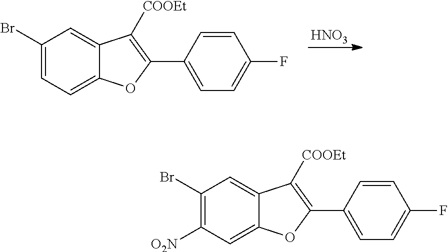

To a solution of ethyl-5-bromo-2-(4-fluorophenyl)benzofuran-3-carboxylate (50 g, 137.6 mmol) in CHCl₃ (500 mL), fuming HNO₃ (50 mL) was added dropwise at −15° C. and the mixture was stirred for 0.5 hours. The reaction mixture was poured into ice water and extracted with CH₂Cl₂. The organic layer was washed with a.q. sat. NaHCO₃ and brine, after removed the most of solvent, the residue obtained was crystallized with petroleum ether: dichloromethane=20:1 to provide product of ethyl 5-bromo-2-(4-fluorophenyl)-6-nitrobenzofuran-3-carboxylate (35 g, 66%)

¹H-NMR (CDCl₃, 400 MHz) δ 8.36 (s, 1H), 8.02˜8.04 (m, 3H), 7.13˜7.18 (m, 2H), 4.36˜4.41 (m, 2H), 1.37 (t, J=4.0 Hz, 3H).

### Step 2-Synthesis of ethyl 6-amino-5-bromo-2-(4-fluorophenyl)benzofuran-3-carboxylate (Compound 411D)

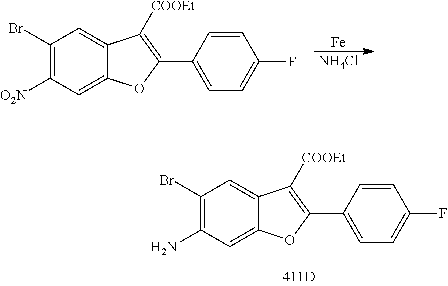

A mixture of ethyl 5-bromo-2-(4-fluorophenyl)-6-nitrobenzofuran-3-carboxylate (52 g, 127 mmol), iron filings (21.3 g, 382.2 mmol) and NH₄Cl (41 g, 764.4 mmol) in MeOH/THF/H₂O (2:2:1, 500 mL) was allowed to stir at reflux for 3 hours. After being filtered and concentrated in vacuo, the residue obtained was purified using column chromatography (petroleum ether:EtOAc:dichloromethane=20:1:20) to provide the pure ethyl 6-amino-5-bromo-2-(4-fluorophenyl)benzofuran-3-carboxylate (compound 411D) (40 g, 82%). ¹H-NMR (CDCl₃, 400 MHz) δ 8.01 (s, 1H), 7.94˜7.98 (m, 2H), 7.08 (t, J=8.0 Hz, 2H), 6.83 (s, 1H), 4.32˜4.36 (m, 2H), 4.18 (s, 2H), 1.35 (t, J=8.0 Hz, 3H). MS (M+H)⁺: 378/380.

### Step 3-Synthesis of 5-Bromo-2-(4-fluoro-phenyl)-6-methanesulfonylamino-benzofuran-3-carboxylic acid ethyl ester (Compound 411E)

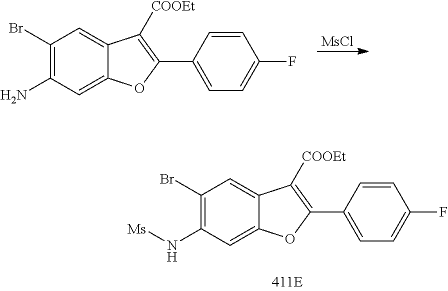

MsCl (31.7 g, 277.5 mmol) was added to a solution of ethyl 6-amino-5-bromo-2-(4-fluorophenypbenzofuran-3-carboxylate (35 g, 92.5 mmol) and pyridine (60 mL) in dry dichloromethane (300 mL) at 0° C. After stirred for about 15 hours at room temperature, the mixture was diluted with water, and extracted with dichloromethane. The organic layer was washed with brine, dried over Na₂SO₄, filtered and concentrated in vacuo, the residue obtained was purified using crystallized with EtOAc to provide the pure product of Compound 411E (35 g, 82%). ¹H-NMR (CDCl₃, 400 MHz) δ 8.27 (s, 1H), 8.01˜8.05 (m, 2H), 7.87 (s, 1H), 7.15˜7.19 (m, 2H), 6.87 (s, 1H), 4.38˜4.43 (m, 2H), 3.00 (s, 3H), 1.40 (t, J=40 Hz, 3H). MS (M+H)⁺: 456/458.

### Step 4-Synthesis of 5-Bromo-2-(4-fluoro-phenyl)-6-methanesulfonylamino-benzofuran-3-carboxylic acid (Compound 411F)

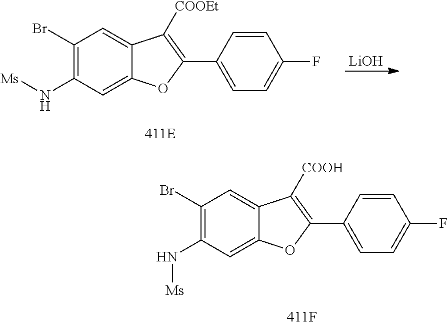

To a solution of Compound 411E (53 g, 0.23 mol) in dioxane/H₂O (5:1, 600 mL) was added LiOH.H₂O (25 g, 1.17 mol), and the mixture was allowed to stir at 100° C. and allowed to stir at this temperature for 3 hours. After being concentrated in vacuo, the residue obtained was dissolved in H₂O, 1 N HCl was added until pH reached 3, and the reaction mixture was extracted with EtOAc. The organic layer was washed with brine, dried over Na₂SO₄ and filtered. The solvent was removed by distillation to provide the crude product of Compound 411F (48 g, 96%). ¹H-NMR (400 MHz, DMSO) δ 13.49 (s, 1H), 9.67 (s, 1H), 8.30 (s, 1H), 8.12˜8.17 (m, 2H), 7.87 (s, 1H), 7.45˜7.50 (m, 2H), 3.16 (s, 3H). MS (M+H)⁺: 428/430.

### Step 5—Synthesis of 5-Bromo-2-(4-fluoro-phenyl)-6-methanesulfonylamino-benzofuran-3-carboxylic acid methylamide (Compound 411G)

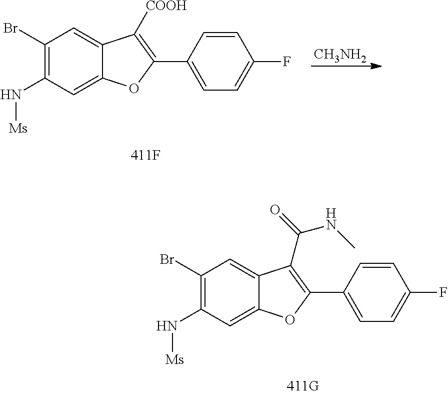

A solution of Compound 411F (33 g, 77 mmol), HOBT (15.6 g, 115.5 mmol) and EDCI (22.2 g, 115.5 mmol) in dry DMF (250 mL) was allowed to stir at room temperature. After 2 hours, Et₃N (50 mL) and CH₃NH₂ (HCl salt, 17.7 g, 231 mmol) was added to the mixture, and the mixture was stirred for about 15 hours. After the solvent was removed, H₂O was added and the reaction mixture was extracted with ethyl acetate. The organic extract was washed with H₂O, brine and concentrated in vacuo and the residue obtained was washed with EtOAc to provide Compound 411G (32 g, 94%). ¹H-NMR (400 MHz, DMSO) δ 9.55 (br s, 1H), 8.46˜8.48 (m, 1H), 8.12˜8.17 (m, 2H), 7.96 (s, 1H), 7.87 (s, 1H), 7.45˜7.50 (m, 2H), 3.16 (s, 3H), 2.93 (d, J—8.4 Hz, 3H). MS (M+H)⁺: 441/443.

### Step 6—Synthesis of 5-Bromo-2-(4-fluoro-phenyl)-6-(methanesulfonyl-methyl-amino)-benzofuran-3-carboxylic acid methylamide (Compound 411H)

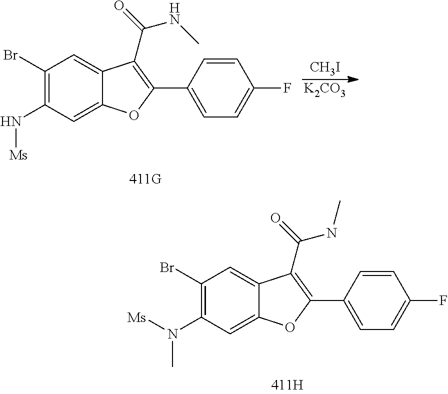

CH₃I (24.3 g, 171 mmol) was added to a mixture of Compound 411G (25 g, 57.1 mmol), K₂CO₃ (19.8 g, 143 mmol) and KI (190 mg, 1.1 mmol) in DMF (100 mL) under N₂ protection. The reaction was allowed to stir at reflux for about 15 hours, then was concentrated in vacuo and the residue obtained was washed with water and EtOAc to provide Compound 411H (24 g, 93%). ¹H-NMR (CDCl₃, 400 MHz) δ 8.16 (s, 1H), 7.88˜7.92 (m, 2H), 7.70 (s, 1H), 7.18˜7.23 (m, 2H), 5.78 (br s, 1H), 3.34 (s, 3H), 3.09 (s, 3H), 3.00 (d, J=4.8 Hz, 3H). MS (M+H)⁺: 455/457.

### Step 7—Synthesis of 1-fluoro-3-methoxy-2-nitrobenzene

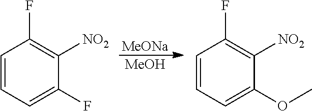

To a solution of 1,3-difluoro-2-nitrobenzene (100 g, 0.63 mol) in MeOH (1.3 L) was added a solution of NaOMe (0.69 mol, in MeOH, freshly prepared from 15.9 g of metal Na and 200 mL of MeOH) slowly at 0° C. The reaction was allowed to stir for about 15 hours at room temperature, then the reaction mixture was concentrated in vacuo and the residue obtained was diluted with EtOAc. The resulting solution was washed with water and brine, dried over Na₂SO₄, filtered and concentrated in vacuo to provide 1-fluoro-3-methoxy-2-nitrobenzene (98 g, 91.4%). ¹H-NMR (CDCl₃, 400 MHz) δ 7.38˜7.44 (m, 1H), 6.72˜6.88 (m, 2H), 3.95 (s, 3H).

### Step 8—Synthesis of 3-Fluoro-2-nitro-phenol

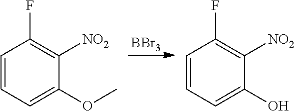

To a solution of 1-Fluoro-3-methoxy-2-nitro-benzene (98 g, 0.57 mol) in dichloromethane (500 mL) was added dropwise a solution of BBr₃ (1 L, 1 M in dichloromethane) at −40° C. The reaction was allowed to stir for about 15 hours at room temperature, then the reaction mixture was slowly poured into ice water (500 mL). The mixture was extracted with EtOAc (300 mL×3), and the combined organic extracts were washed with 5% aqueous NaHCO₃, brine, dried over Na₂SO₄, filtered and concentrated in vacuo to provide 3-fluoro-2-nitro-phenol (85 g, 95%). ¹H-NMR (CDCl₃, 400 MHz) δ 7.43˜7.49 (m, 1H), 6.88 (d, J=8.0 Hz, 1H), 6.73˜6.78 (m, 1H).

### Step 9—Synthesis of 2-Amino-3-fluoro-phenol

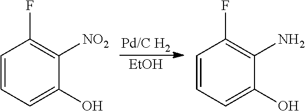

3-Fluoro-2-nitro-phenol (38 g, 0.24 mol) was dissolved in EtOH and to the resulting solution was added palladium on carbon (5 g, 10% Pd). The reaction was put under H₂ atmosphere (1 atm) and allowed to stir for 3 hours at room temperature. The reaction mixture was filtered and the collected palladium was washed with EtOH. The filtrate and washing was combined and concentrated in vacuo to provide 2-amino-3-fluoro-phenol (26 g, 85.7%). ¹H-NMR (DMSO, 400 MHz) δ 9.43 (s, 1H), 6.42˜6.53 (m, 2H), 6.32˜6.42 (m, 1H), 4.34 (s, 2H).

### Step 10—Synthesis of 2-(5-Bromo-2-methoxy-phenyl)-4-fluoro-benzooxazole

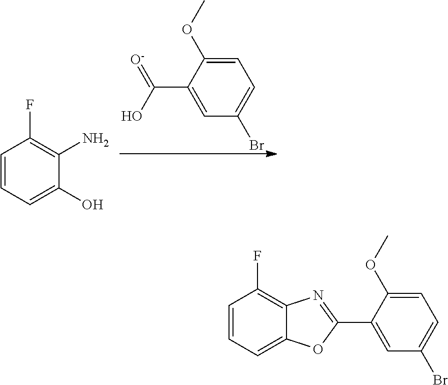

To a solution of 2-amino-3-fluoro-phenol (9 g 70.8 mmol) in 10 mL of polyphosphoric acid was added 5-bromo-2-methoxybenzoic acid (16.3 g, 70.8 mmol), and the resulting mixture was heated to 140° C. and allowed to stir at this temperature for 4 hours. The reaction mixture was then poured into ice water (50 mL) and extracted with EtOAc. The organic extract was concentrated in vacuo and the resulting residue was purified using column chromatography on silica gel (petroleum ether:EtOAc=10:1) to provide 2-(5-bromo-2-methoxy-phenyl)-4-fluoro-benzooxazole (16 g, 82%) as a solid. ¹H-NMR (CDCl₃, 400 MHz) δ 8.29 (d, J=2.4 Hz, 1H), 7.57˜7.54 (m, 1H), 7.40 (d, J=8.0 Hz, 1H), 7.27˜7.33 (m, 1H), 7.07 (m, 1H), 6.96 (d, J=9.2 Hz, 1H), 3.99 (s, 3H).

### Step 11—Synthesis of 4-Fluoro-2-[2-methoxy-5-(4, 4,5,5-tetramethyl-[1,3,2]dioxaborolan-2-yl)-phenyl]-benzooxazole

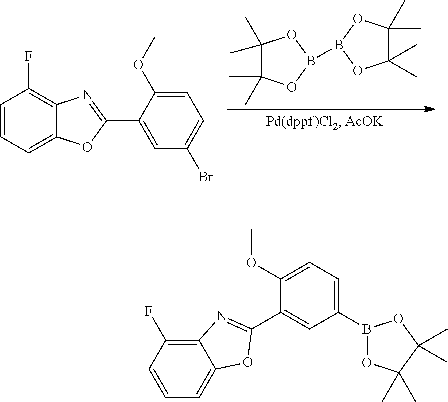

To a solution of 2-(5-bromo-2-methoxy-phenyl)-4-fluoro-benzooxazole (18.4 g, 57.1 mmol) in DMF (10 mL), bis(pinacolato)diboron (17.4 g, 68.5 mmol) and AcOK (10 g, 1.14 mmol) was added, and the resulting mixture was heated to 80° C. and allowed to stir at this temperature for 3 hours. The reaction mixture was concentrated in vacuo and the residue obtained was dissolved in dichloromethane and filtered through celite. The filtrate was washed with H₂O and brine, dried over Na₂SO₄, filtered and concentrated in vacuo. The residue obtained was purified using column chromatography (petroleum ether:EtOAc=10:1) to provide 4-fluoro-2-[2-methoxy-5-(4, 4,5,5-tetramethyl-[1, 3, 2]dioxaborolan-2-yl)-phenyl]-benzooxazole (10 g, 54%) as a solid. ¹H-NMR (CDCl₃, 400 MHz) δ 8.53 (d, J=1.6 Hz, 1H), 7.85˜7.92 (m, 1H), 7.44 (d, J=8.0 Hz, 1H), 7.20˜7.28 (m, 1H), 6.96˜7.05 (m, 2H), 3.97 (s, 3H), 1.29 (s, 12H).

### Step 12—Synthesis of 5-[3-(4-Fluoro-benzooxazol-2-yl)-4-methoxy-phenyl]-2-(4-fluoro-phenyl)-6-(methanesulfonyl-methyl-amino)-benzofuran-3-carboxylic acid methylamide

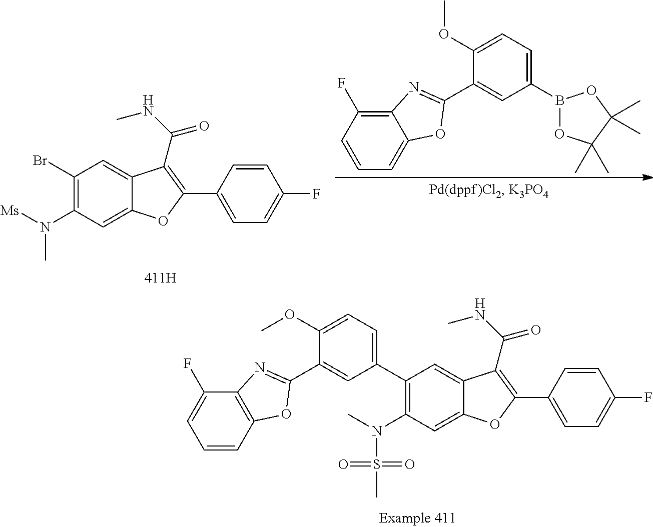

To a solution of 4-fluoro-2-[2-methoxy-5-(4, 4,5,5-tetramethyl-[1, 3, 2]dioxaborolan-2-yl)-phenyl]-benzooxazole (5.27 g, 14.3 mmol) and Compound 411H (5 g, 11.0 mmol) in DMF (150 mL) was added Pd(dppf)Cl₂ (200 mg) and K₃PO₄ (4.66 g, 22.0 mmol) under N₂ protection. The resulting mixture was heated to 100° C. and allowed to stir at this temperature for 10 hours, and then the reaction mixture was cooled to room temperature and concentrated in vacuo. The residue obtained was dissolved in dichloromethane and filtered through celite. The filtrate was washed with water, brine, dried over Na₂SO₄ and concentrated in vacuo. The residue obtained was purified using flash column chromatography (petroleum ether:EtOAc=4:1 to 2:1) and crystallized from dichloromethane:EtOAc (5:1) to provide the target compound (3.8 g, 56%) was obtained as white solid. ¹H-NMR (CDCl₃, 400 MHz) δ 8.21 (d, J=2.0 Hz, 1H), 7.91˜7.95 (m, 2H), 7.83 (s, 1H), 7.68 (d, J=2.0 Hz, 1H), 7.66 (s, 1H), 7.39 (d, J=8.0 Hz, 1H), 7.14˜7.27 (m, 4H), 7.06 (t, J=8.4 Hz, 1H), 5.95 (br s, 1H), 4.06 (s, 3H), 3.14 (s, 3H), 2.99 (d, J=4.8 Hz, 3H), 2.77 (s, 3H).

### Example 412

### 5-(5-(4-fluorobenzo[d]oxazol-2-yl)thiophen-2-yl)-2-(4-fluorophenyl)-N-methyl-6-(N-methylmethylsulfonamido)benzofuran-3-carboxamide

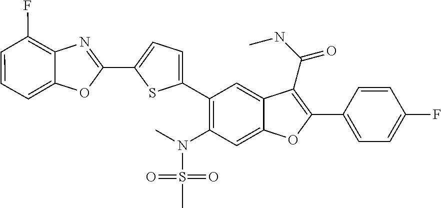

### Step 1—Synthesis of 2-(4-fluorophenyl)-N-methyl-6-(N-methylmethylsulfonamido)-5-(4, 4,5,5-tetramethyl-1,3,2-dioxaborolan-2-yl)benzofuran-3-carboxamide (Compound 411J)

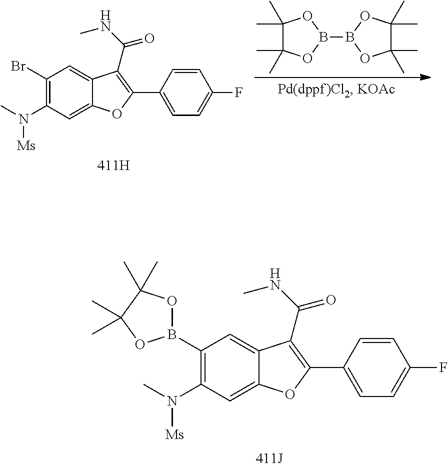

To a degassed solution of Compound 411H (1.0 g, 2.20 mmol) and pinacol diborane (2.79 g, 10.98 mmol) in 1,4-Dioxane (25 mL) was added KOAc (647 mg, 6.59 mmol) under N₂ and the resulting reaction was allowed to stir for 4 hours. Pd(dppf)Cl₂ (60 mg) was then added and the reaction was stirred for another 30 minutes. The reaction flaski was then put into a pre-heated oil-bath at 130° C. and stirred for another 1 hour under N₂. The reaction mixture was cooled to room temperature, then concentrated in vacuo and extracted with EtOAc. The organic extract was washed with brine, dried over Na₂SO₄, filtered and concentrated in vacuo. The resulting residue was purified using flash column chromatography on silica gel (petroleum ether:EtOAc=5:1 to 2:1) to provide Compound 411J as white solid (700 mg, 64%). ¹H-NMR (CDCl₃, 400 MHz) δ 8.17 (s, 1H), 7.87˜7.91 (m, 2H), 7.52 (s, 1H), 7.11 (t, J=7.6 Hz, 2H), 5.81 (d, J=2.8 Hz, 1H), 3.30 (s, 3H), 2.97 (d, J=5.2 Hz, 3H), 2.90 (s, 3H), 1.31 (s, 12H).

### Step 2—Synthesis of 5-(5-(4-fluorobenzo[d]oxazol-2-yl)thiophen-2-yl)-2-(4 fluorophenyl)-N-methyl-6-(N-methylmethylsulfonamido)benzofuran-3-carboxamide

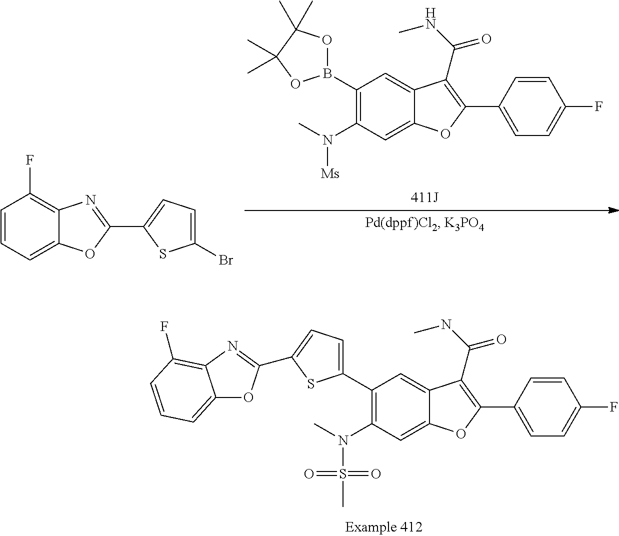

To a degassed solution of Compound 411J (100 mg, 0.2 mmol) and 2-(5-bromothiophen-2-yl)-4-fluorobenzo[d]oxazole (53 mg, 0.2 mmol, prepared using the methods described in Example 1) in dry DMF (3 mL) was added Pd(dppf)Cl₂ (10 mg) and K₃PO₄ (120 mg, 0.4 mmol) under N₂ protection. The reaction was heated to 100° C. and allowed to stir at this temperature for about 15 hours, then was cooled to room temperature and filtered. The filtrate was washed with H₂O, brine, dried over Na₂SO₄, filtered and concentrated in vacuo. The residue obtained was purified using prep-HPLC to provide the target compound (68 mg, 57.6%). ¹H-NMR (CDCl₃, 400 MHz) δ 8.04 (s, 1H), 7.86˜7.89 (m, 3H), 7.82 (s, 1H), 7.69 (s, 1H), 7.55 (s, 1H), 7.23˜7.34 (m, 1H), 7.13 (t, J=8.0 Hz, 2H), 7.03 (t, J=8.8 Hz, 1H), 3.18 (s, 3H), 2.93 (s, 3H), 2.82 (s, 3H). MS (M+H)⁺: 595.

### Example 413

### 5-(4(1H-indol-2-yl)pyridin-2-yl)-2-(4-fluorophenyl)-N-methyl-6-(N-methylmethylsulfonamido)benzofuran-3-carboxamide

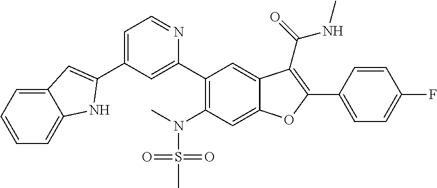

### Step 1—Synthesis of tert-butyl 1H-indole-1-carboxylate

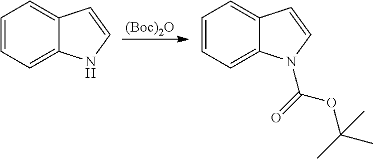

To a solution of indole (1 g, 8.5 mmol) and (Boc)₂O (2.2 g, 10.2 mmol) in dichloromethane (10 mL) was added DMAP (100 mg, 0.85 mmol) at room temperature, and the mixture was stirred for 3 hours. Water was added, extracted with dichloromethane and washed with brine, dried over Na₂SO₄. After being concentrated in vacuo, the residue obtained was purified using column chromatography (petroleum ether:EtOAc=20:1) to provide tert-butyl

1H-indole-1-carboxylate (1.8 g, 96%). ¹H-NMR (CDCl₃, 400 MHz) δ 8.13 (d, J=8.0 Hz, 1H), 7.54˜7.59 (m, 2H), 7.20˜7.32 (m, 2H), 6.56 (t, J=1.8 Hz, 1H), 1.67 (s, 9H).

### Step 2—Synthesis of 1-(tert-butoxycarbonyl)-1H-indol-2-ylboronic acid

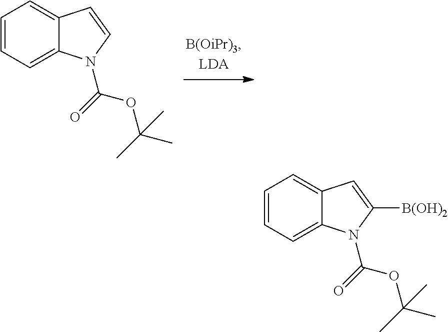

To a solution of tert-butyl 1H-indole-1-carboxylate (1 g, 4.61 mmol) and B(i-PrO)₃ (1.61, 6.91 mmol) in THF (7 mL) was added LDA (3.5 mL, 6.91 mmol) at 0° C., Then warmed up to room temperature and stirred for 30 minutes. 2N HCl was added to acidified the solution until pH=7, extracted with ethyl acetate and washed with brine, dried over Na₂SO₄. After being concentrated in vacuo, the residue obtained was purified using column chromatography (petroleum ether:EtOAc=10:1 to 2:1) to provide 1-(tert-butoxycarbonyl)-1H-indol-2-ylboronic acid (0.5 g, 45%). ¹H-NMR (DMSO, 400 MHz) δ 8.16 (s, 1H), 8.05 (d, J=8.8 Hz, 1H), 7.52 (d, J=8.8 Hz, 1H), 7.24 (t, J=7.2 Hz, 1H), 7.16 (t, J=7.2 Hz, 1H), 6.59 (s, 1H), 1.57 (s, 9H).

### Step 3—Synthesis of tert-butyl 2-(2-chloropyridin-4-yl)-1H-indole-1-carboxylate

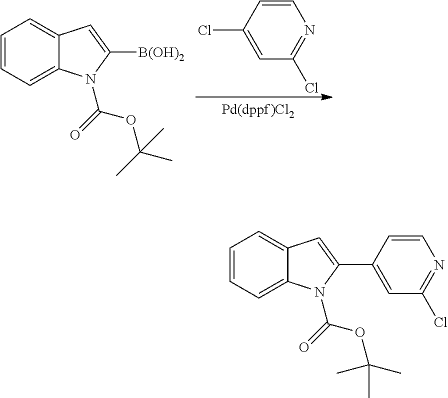

To a mixture of 1-(tert-butoxycarbonyl)-1H-indol-2-ylboronic acid (400 mg, 1.56 mmol), 2-Chloro-4-bromopyridine (200 mg, 1.04 mmol) and K₃PO₄.3H₂O (830 mg, 3.12 mmol) in DMF (6 mL), under nitrogen atmosphere, was added Pd(dppf)Cl₂ (60 mg). The reaction was heated to 90° C. and allowed to stir at this temperature for 5 hours. Water was added, the solution was extracted with ethyl acetate and the organic layer was washed with brine, dried over Na₂SO₄, filtered and concentrated in vacuo. The residue obtained was purified using prep-TLC (petroleum ether:EtOAc=5:1) to provide tert-butyl 2-(2-chloropyridin-4-yl)-1H-indole-1-carboxylate (300 mg, 88%). MS (M+H)⁺: 328/330.

### Step 4—Synthesis of 2-(2-chloropyridin-4-yl)-11′-indole

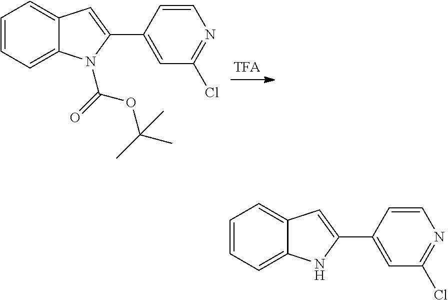

To a 0° C. solution of tert-butyl 2-(2-chloropyridin-4-yl)-1H-indole-1-carboxylate (328 g, 1.0 mmol) in dichloromethane (5 mL) was added TFA (0.5 mL) dropwise. The reaction was allowed to warm to room temperature with stirring, then was allowed to stir for an additional 1 hour. Water was added and the resulting solution was extracted with dichloromethane and the organic phase was washed with brine, dried over Na₂SO₄, filtered and concentrated in vacuo to provide 2-(2-chloropyridin-4-yl)-1H-indole (150 mg, 66%) was obtained. ¹H-NMR (CDCl₃, 400 MHz) δ 8.48 (s, 1H), 8.41 (d, J=4.4 Hz, 1H), 7.67 (d, J=8.8 Hz, 1H), 7.56 (s, 1H), 7.42˜7.46 (m, 2H), 7.30 (d, J=8.4 Hz, 1H), 7.17 (t, J=8.0 Hz, 1H), 7.06 (s, 1H).

### Step 5—Synthesis of 5-(4-(1H-indol-2-yl)pyridin-2-yl)-2-(4-fluorophenyl)-N-methyl-6-(N-methylmethylsulfonamido)benzofuran-3-carboxamide

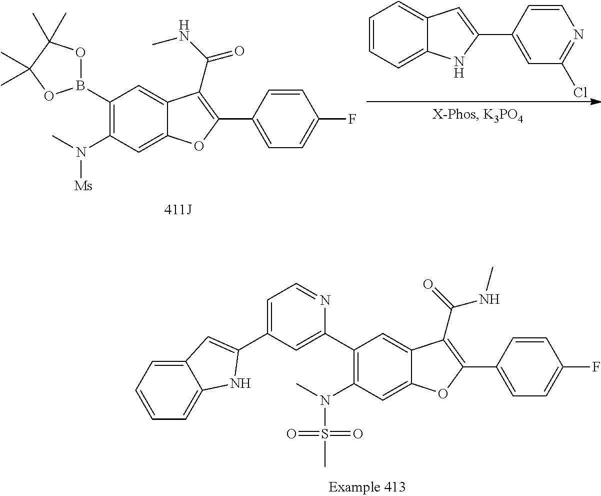

A mixture of 2-(2-chloropyridin-4-yl)-1H-indole (34 mg, 0.15 mmol), Compound 411J (50 mg, 0.1 mmol), K₃PO₄.3H₂O (80 mg, 0.3 mmol), Pd₂(dba)₃ (9.15 mg, 0.01 mmol) and X-Phos (9.50 mg, 0.02 mmol) in 1,4-dioxane (2 mL) and H₂O (0.5 mL) was heated to 110° C. and allowed to stir at this temperature for 12 hours. Water was added and the reaction mixture was extracted with ethyl acetate. The organic extract was and washed with brine, dried over Na₂SO₄, filtered and concentrated in vacuo and the residue obtained was purified using prep-HPLC to provide the target compound (40 mg, 69%). ¹H-NMR (CDCl₃, 400 MHz) δ 9.48 (s, 1H), 8.64 (d, J=5.2 Hz, 1H), 8.09 (d, J=2.0 Hz, 1H), 8.05 (s, 1H), 7.94˜7.97 (m, 2H), 7.64 (d, J=8.0 Hz, 1H), 7.55 (d, J=5.2 Hz, 1H), 7.53 (s, 1H), 7.43 (d, J=8.0 Hz, 1H), 7.11˜7.25 (m, 4H), 7.01 (s, 1H), 6.16 (s, 1H), 3.09 (d, J=0.8 Hz, 3H), 3.03 (s, 3H), 2.98 (d, J=4.8 Hz, 3H). MS (M+H)⁺: 569.

The following compounds of the present invention were made using the methods described above in Examples 411-413 and using the appropriate reactants and/or reagents.

| Exam- | | | MS |
|---|---|---|---|
| ple | Structure | NMR | (M + H)⁺ |
| 414 | 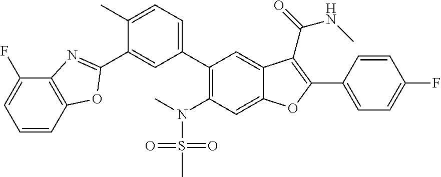 | ¹H-NMR (CDCl₃, 400 MHz) δ 8.21 (s, 1H), 7.87~7.90 (m, 2H), 7.79 (s, 1H), 7.57 (s, 1H), 7.51 (d, J = 1.6 Hz, 1H), 7.49 (d, J = 2.0 Hz, 1H), 7.38 (d, J = 8.0 Hz, 1H), 7.22~7.27 (m, 1H), 7.12~7.16 (m, 2H), 7.00~7.05 (m, 1H), 5.83 (d, J = 4.8 Hz, 1H), 3.11 (s, 3H), 2.93 (d, J = 4.8 Hz, 3H), 2.82 (s, 3H), 2.66 (s, 3H). | 602 |
| 415 | 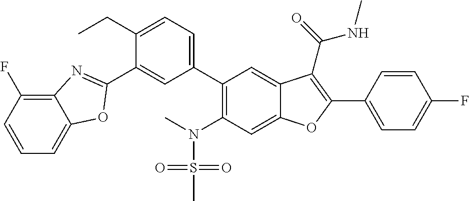 | ¹H-NMR (CDCl₃, 400 MHz): δ 8.21 (d, 1H), 7.97~7.92 (m, 2H), 7.84 (s, 1H), 7.63~7.58 (m, 2H), 7.47 (d, 1H), 7.41 (d, 1H), 7.34~7.27 (m, 1H), 7.20 (t, J = 8.7 Hz, 2H), 7.08 (t, J = 8.7 Hz, 1H), 5.90 (d, 1H), 3.33~3.26 (m, 2H), 3.17 (s, 3H), 2.99 (d, 3H), 3.14 (s, 1H), 1.34 (t, 3H). | 616 |
| 416 | 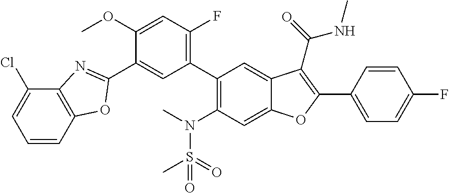 | H-NMR (CDCl₃, 400 MHz) δ 8.16 (d, J = 8.4 Hz, 1H), 7.87~7.90 (m, 2H), 7.80 (s, 1H), 7.60 (s, 1H), 7.42 (d, J = 8.0 Hz, 1H), 7.28 (d, J = 8.0 Hz, 1H), 7.21 (d, J = 8.0 Hz, 1H), 7.13 (t, J = 8.4 Hz, 2H), 6.81 (d, J = 11.6 Hz, 1H), 5.94 (d, J = 3.6 Hz, 1H), 3.99 (s, 3H), 3.20 (s, 3H), 2.94 (d, J = 4.8 Hz, 3H), 2.61 (s, 3H). | 653 |
| 417 | 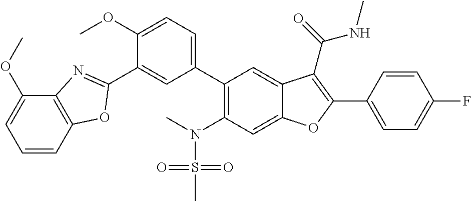 | ¹H-NMR (CDCl₃, 400 MHz) δ 8.16 (s, 1H), 7.55~7.90 (m, 5H), 7.09~7.16 (m, 5H), 5.97 (s, 1H), 3.96~3.99 (m, 6H), 3.08 (s, 3H), 2.96 (s, 3H), 2.69 (s, 3H). | 630 |
| 418 | 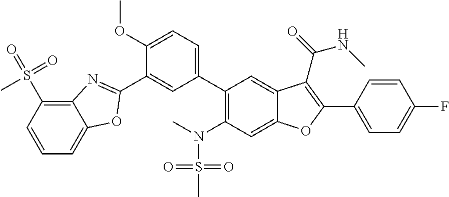 | ¹H-NMR (CDCl₃, 400 MHz) δ 8.34 (s, 1H), 7.85~7.97 (m, 5H), 7.69 (d, J = 8.0 Hz, 1H), 7.62 (s, 1H), 7.47~7.51 (m, 1H), 7.15~7.25 (m, 3H), 6.05 (br s, 1H), 4.05 (s, 3H), 3.47 (s, 3H), 3.17 (s, 3H), 2.96 (d, J = 4.8 Hz, 3H), 2.90 (s, 3H). | 678 |
| 419 | 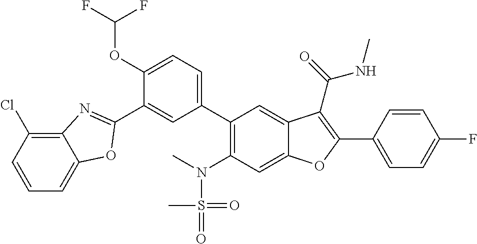 | ¹H-NMR (CDCl₃, 400 MHz) δ 8.31 (s, 1H), 7.86~7.89 (m, 2H), 7.81 (s, 1H), 7.65 (d, J = 8.4 Hz, 1H), 7.57 (s, 1H), 7.46 (d, J = 8.0 Hz, 1H), 7.38 (d, J = 8.8 Hz, 1H), 7.32 (d, J = 8.8 Hz, 1H), 7.26 (t, J = 8.0 Hz, 1H), 7.14 (t, J = 8.4 Hz, 2H), 6.56~6.93 (m, 1H), 5.91 (d, J = 3.6 Hz, 1H), 3.10 (s, 3H), 2.94 (d, J = 3.6 Hz, 3H), 2.79 (s, 3H). | 671 |
| 420 | 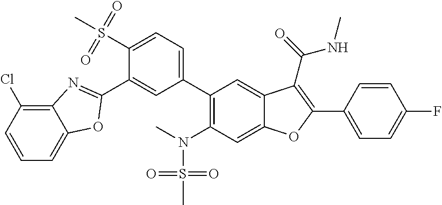 | ¹H-NMR (CDCl₃, 400 MHz) δ 8.31 (d, J = 8.0 Hz, 1H), 8.05 (s, 1H), 7.83~7.88 (m, 4H), 7.60 (s, 1H), 7.47 (d, J = 8.0 Hz, 1H), 7.36 (d, J = 7.2 Hz, 1H), 7.30 (d, J = 8.0 Hz, 1H), 7.14~7.16 (m, 2H), 5.76 (d, J = 2.4 Hz, 1H), 3.67 (s, 3H), 3.09 (s, 3H), 2.93 (d, J = 4.8 Hz, 3H), 2.88 (s, 3H). | 683 |
| 421 | 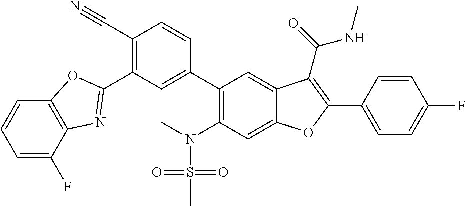 | ¹H-NMR (CDCl₃, 400 MHz) δ 8.50 (s, 1H), 7.85~7.91 (m, 4H), 7.75 (d, J = 8.0 Hz, 1H), 7.58 (s, 1H), 7.41~7.45 (m, 1H), 7.31~7.36 (m, 1H), 7.15 (d, J = 8.4 Hz, 2H), 7.08 (t, J = 8.8 Hz, 2H), 5.81 (d, J = 6.4 Hz, 1H), 3.15 (s, 3H), 2.94 (d, J = 4.4 Hz, 3H), 2.81 (s, 3H). | 613 |
| 422 | 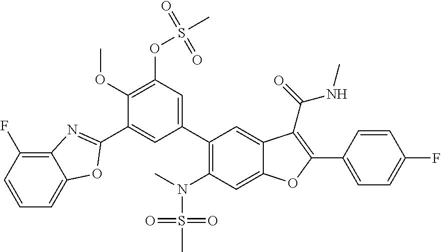 | ¹H-NMR (CDCl₃, 400 MHz) 7.94~7.99 (m, 2H), 7.91~7.93 (m, 2H), 7.56~7.60 (m, 1H), 7.45~7.47 (m, 1H), 7.33~7.38 (m, 2H), 7.20~7.24 (m, 2H), 7.11 (t, J = 4.4 Hz, 1H), 5.89~5.94 (m, 1H), 4.04 (s, 3H), 3.53 (s, 3H), 3.10 (s, 3H), 3.01 (d, J = 4.8 Hz, 3H), 2.94 (s, 3H). | 712 |
| 423 | 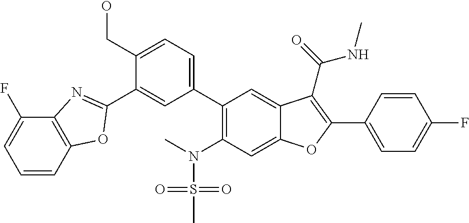 | ¹H-NMR (CDCl₃, 400 MHz) δ 8.30 (s, 1H), 7.84~7.86 (m, 2H), 7.79 (s, 1H), 7.51~7.59 (m, 3H), 7.36 (d, J = 4.0 Hz, 1H), 7.34~7.35 (m, 1H), 7.13~7.28 (m, 2H), 7.07~7.11 (m, 1H), 5.91 (d, J = 4.0 Hz, 1H), 5.67 (brs, 1H), 4.87 (s, 1H), 3.08 (s, 3H), 2.91 (d, J = 4.0 Hz, 3H), 2.67 (s, 3H). | 618 |
| 424 | 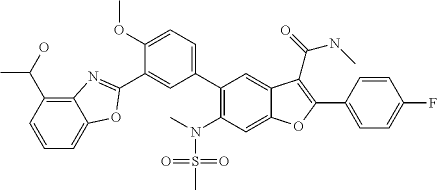 | ¹H-NMR (CDCl₃, 400 MHz) δ 8.16 (d, J = 2.4 Hz, 1H), 7.85~7.88 (m, 2H), 7.79 (s, 1H), 7.60~7.63 (m, 1H), 7.56 (s, 1H), 7.47~7.49 (d, J = 8.0 Hz, 1H), 7.29~7.33 (m, 1H), 7.22~7.24 (m, 1H), 7.09~7.17 (m, 3H), 6.16 (d, J = 4.4 Hz, 1H), 5.62 (s, 1H), 5.37 (d, J = 6.0 Hz, 1H), 3.99 (s, 3H), 3.13 (s, 3H), 2.96 (d, J = 4.4 Hz, 3H), 2.76 (s, 3H), 1.65 (d, J = 4.4 Hz, 3H). | 644 |
| 425 | 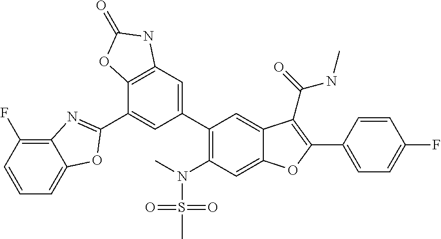 | ¹H-NMR (CDCl₃, 400 MHz) δ 9.58 (br s, 1H), 7.81 (s, 1H), 7.71 (br s, 3H), 7.49 (br s, 1H), 7.41 (s, 1H), 7.30~7.37 (m, 3H), 7.03 (t, J = 8.4 Hz, 1H), 6.92 (t, J = 8.0 Hz, 2H), 3.19 (d, J = 3.6 Hz, 3H), 3.01 (s, 3H), 3.00 (s, 3H). | 645 |
| 426 | 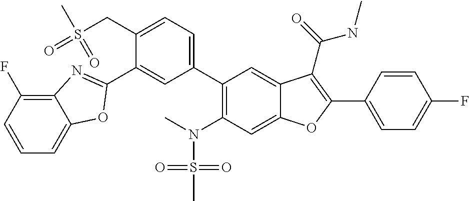 | ¹H-NMR (CDCl₃, 400 MHz) δ 8.33 (s, 1H), 7.85~8.33 (m, 3H), 7.72 (d, J = 8.0 Hz, 1H), 7.61~7.64 (m, 1H), 7.58 (s, 1H), 7.26~7.35 (m, 2H), 7.13~7.18 (m, 2H), 7.05 (t, J = 8.8 Hz, 1H), 5.80 (s, 1H), 5.35 (s, 2H), 3.16 (s, 3H), 2.94 (d, J = 8.0 Hz, 3H), 2.92 (s, 3H), 2.65 (s, 3H). | 680 |
| 427 | 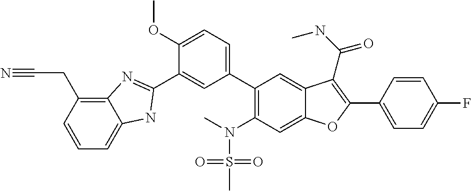 | ¹H-NMR (CDCl₃, 400 MHz) δ 8.13 (d, J = 6.0 Hz, 1H), 7.82~7.85 (m, 2H), 7.78 (s, 1H), 7.58~7.61 (m, 1H), 7.49~7.53 (m, 2H), 7.30~7.36 (m, 2H), 7.08~7.19 (m, 3H), 5.97 (d, J = 4.4 Hz, 1H), 4.12 (s, 2H), 3.99 (s, 3H), 3.08 (s, 3H), 2.89 (d, J = 8.0 Hz, 3H), 2.71 (s, 3H). | 639 |
| 428 | 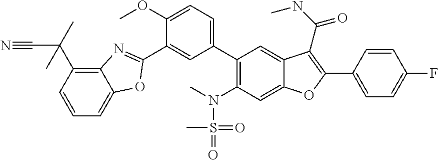 | ¹H-NMR (CDCl₃, 400 MHz) δ 8.24 (d, J = 2.4 Hz, 1H), 7.91~7.94 (m, 2H), 7.81 (s, 1H), 7.58~7.64 (m, 3H), 7.50~7.52 (m, 1H), 7.32~7.36 (m, 1H), 7.15~7.21 (m, 3H), 6.05 (d, J = 4.8 Hz, 1H), 4.04 (s, 3H), 3.21 (s, 3H), 2.97 (d, J = 4.8 Hz, 3H), 2.76 (s, 3H), 2.06 (s, 6H). | 667 |
| 429 | 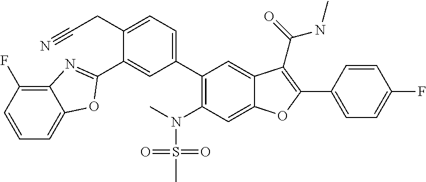 | ¹H-NMR (CDCl₃, 400 MHz) δ 8.69 (s, 1H), 7.89~7.93 (m, 3H), 7.73 (d, J = 8.0 Hz, 1H), 7.70 (d, J = 8.0 Hz, 1H), 7.63 (s, 1H), 7.31~7.40 (m, 2H), 7.18~7.24 (m, 2H), 7.08~7.12 (m, 1H), 5.91 (d, J = 8.0 Hz, 1H), 4.64 (s, 2H), 3.17 (s, 3H), 2.97 (d, J = 8.0 Hz, 3H), 2.78 (s, 3H). | 627 |
| 430 | 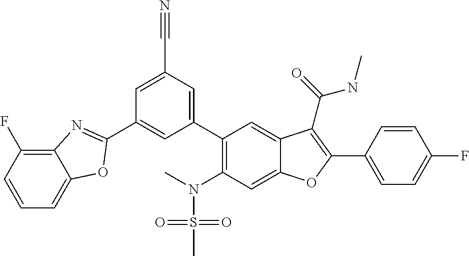 | ¹H-NMR (CDCl₃, 400 MHz) δ 8.52 (d, J = 9.6 Hz, 2H), 7.80~7.89 (m, 4H), 7.59 (s, 1H), 7.31~7.46 (m, 3H), 7.04~7.10 (m, 2H), 5.79 (d, J = 4.0 Hz, 1H), 3.18 (s, 3H), 2.94 (d, J = 4.4 Hz, 3H), 2.78 (s, 3H). | 613 |
| 431 | 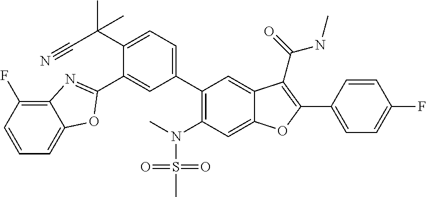 | ¹H-NMR (CDCl₃, 400 MHz) δ 7.72~7.88 (m, 3H), 7.71 (d, J = 4.0 Hz, 1H), 7.66 (d, J = 12.0 Hz, 1H), 7.60 (d, J = 8.0 Hz, 1H), 7.56 (s, 1H), 7.37 (d, J = 8.0 Hz, 1H), 7.27~7.32 (m, 1H), 7.11~7.19 (m, 2H), 7.05 (t, J = 8.8 Hz, 1H), 5.80 (d, J = 4.0 Hz, 1H), 3.07 (s, 3H), 2.91 (d, J = 8.0 Hz, 3H), 2.80 (s, 3H), 1.94 (s, 6H). | 655 |
| 432 | 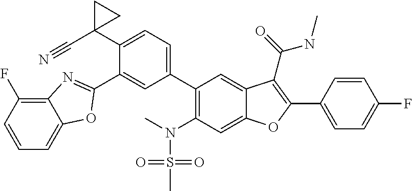 | ¹H-NMR (CDCl₃, 400 MHz) δ 8.40 (s, 1H), 7.97 (t, J = 5.6 Hz, 2H), 7.88 (s, 1H), 7.75 (d, J = 7.2 Hz, 1H), 7.68 (d, J = 7.2 Hz, 2H), 7.50 (d, J = 8.0 Hz, 1H), 7.37~7.42 (m, 1H), 7.21~7.28 (m, 2H), 7.13~7.17 (m, 1H), 5.93 (s, 1H), 3.18 (s, 3H), 3.02 (d, J = 4.8 Hz, 3H), 2.87 (s, 3H), 1.89~1.93 (m, 2H), 0.85~0.91 (m, 2H). | 653 |
| 433 | 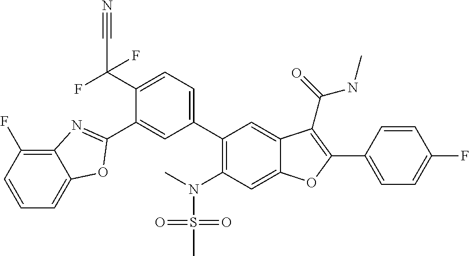 | ¹H-NMR (CDCl₃, 400 MHz) δ 8.38 (s, 1H), 7.93 (d, J = 8.0 Hz, 1H), 7.81~7.89 (m, 4H), 7.59 (s, 1H), 7.40 (d, J = 4.0 Hz, 1H), 7.26~7.37 (m, 1H), 7.14 (d, J = 8.0 Hz, 2H), 7.05~7.09 (m, 1H), 5.79 (d, J = 4.0 Hz, 1H), 3.31 (s, 3H), 2.93 (d, J = 8.0 Hz, 3H), 2.84 (s, 3H). | 663 |
| 434 | 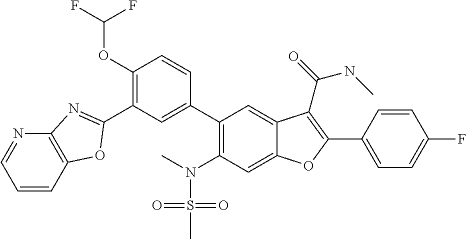 | ¹H-NMR (CDCl₃, 400 MHz) δ 8.59 (s, 1H), 8.34 (d, J = 2.0 Hz, 1H), 7.86~7.89 (m, 3H), 7.83 (s, 1H), 7.68 (d, J = 8.4 Hz, 1H), 7.58 (s, 1H), 7.41 (d, J = 8.4 Hz, 1H), 7.30 (s, 1H), 7.15 (t, J = 8.0 Hz, 2H), 6.59~7.96 (m, 1H), 5.85 (d, J = 4.4 Hz, 1H), 3.14 (s, 3H), 2.94 (d, J = 5.2 Hz, 3H), 2.75 (s, 3H). | 637 |
| 435 | 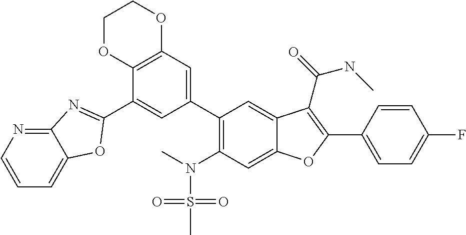 | ¹H-NMR (CDCl₃, 400 MHz) δ 8.54 (s, 1H), 7.78~7.89 (m, 5H), 7.57 (d, J = 2.8 Hz, 1H), 7.26 (d, J = 4.0 Hz, 2H), 7.15~7.19 (m, 2H), 5.85 (br s, 1H), 4.37~4.48 (m, 4H), 3.15 (s, 3H), 2.95 (s, 3H), 2.74 (s, 3H). | 629 |
| 436 | 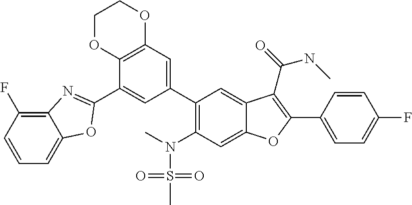 | ¹H-NMR (CDCl₃, 400 MHz) δ 7.87~7.90 (m, 2H), 7.76 (d, J = 8.0 Hz, 2H), 7.57 (s, 1H), 7.33 (d, J = 8.0 Hz, 1H), 7.22~7.25 (m, 1H), 6.99~7.15 (m, 4H), 5.86 (br s, 1H), 4.35~4.48 (m, 4H), 3.13 (s, 3H), 2.94 (d, J = 4.8 Hz, 3H), 2.72 (s, 3H). | 646 |
| 437 | 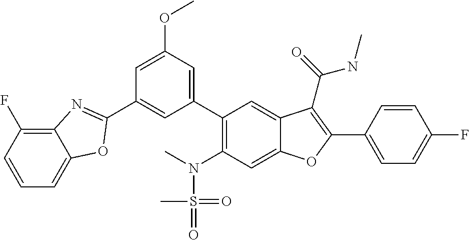 | ¹H-NMR (CDCl₃, 400 MHz) δ 7.86~7.90 (m, 3H), 7.80 (s, 1H), 7.76 (d, J = 1.6 Hz, 1H), 7.56 (s, 1H), 7.34 (d, J = 8.0 Hz, 1H), 7.22~7.27 (m, 1H), 7.19~7.22 (m, 1H), 7.11~7.16 (m, 2H), 7.01~7.05 (m, 1H), 3.90 (s, 3H), 3.08 (s, 3H), 2.93 (d, J = 4.8 Hz, 3H), 2.72 (s, 3H). | 618 |
| 438 | 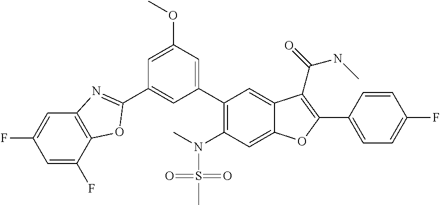 | ¹H-NMR (CDCl₃, 400 MHz) δ 7.88~7.96 (m, 4H), 7.79 (s, 1H), 7.63 (s, 1H), 7.19~7.23 (m, 2H), 7.26~7.31 (m, 2H), 6.90~6.95 (m, 1H), 5.93 (d, J = 4.0 Hz, 1H), 3.97 (s, 3H), 3.16 (s, 3H), 3.00 (d, J = 4.8 Hz, 3H), 2.80 (s, 3H). | 636 |
| 439 | 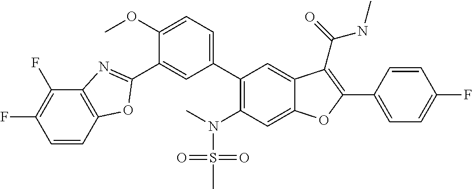 | ¹H-NMR (CDCl₃, 400 MHz) δ 8.17 (s, 1H), 7.93 (t, J = 6.8 Hz, 2H), 7.86 (s, 1H), 7.61~7.70 (m, 3H), 7.44 (t, J = 7.8 Hz, 1H), 7.17~7.23 (m, 3H), 5.96 (d, J = 3.6 Hz, 1H), 4.07 (s, 3H), 3.16 (s, 3H), 2.99 (d, J = 4.4 Hz, 3H), 2.80 (s, 3H). | 636 |
| 440 | 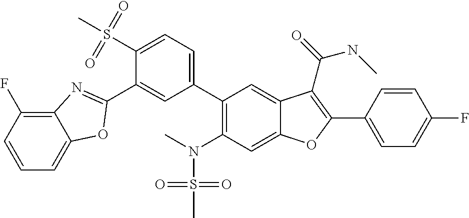 | ¹H-NMR (MeOD, 400 MHz) δ 8.23~8.25 (m, 1H), 8.05 (s, 1H), 7.88~7.93 (m, 2H), 7.86 (s, 1H), 7.71 (s, 2H), 7.48~7.50 (m, 2H), 7.38~7.39 (m, 1H), 7.16~7.21 (m, 2H), 3.56 (s, 3H), 3.18 (s, 3H), 2.87 (s, 3H), 2.85 (s, 3H). | 666 |
| 441 | 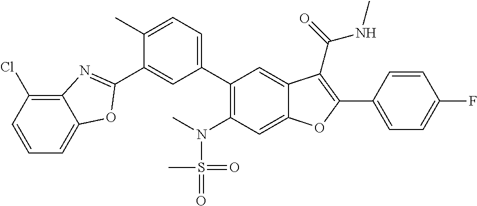 | ¹H-NMR (CDCl₃, 400 MHz) δ 8.35 (s, 1H), 7.92~7.94 (m, 2H), 7.84 (s, 1H), 7.63 (s, 1H), 7.54~7.57 (m, 1H), 7.49 (d, J = 8.0 Hz, 1H), 7.43 (d, J = 8.0 Hz, 1H), 7.36 (d, J = 7.6 Hz, 1H), 7.26~7.30 (m, 1H), 7.19 (t, J = 8.4 Hz, 2H), 5.89 (d, J = 4.0 Hz, 1H), 3.15 (s, 3H), 2.98 (d, J = 4.8 Hz, 3H), 2.87 (s, 3H), 2.73 (s, 3H). | 619 |
| 442 | 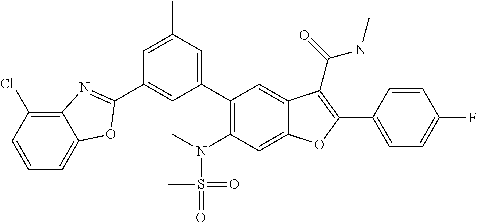 | ¹H-NMR (CDCl₃, 400 MHz) δ 8.10 (t, J = 0.8 Hz, 1H), 7.90~7.87 (m, 2H), 7.79 (s, 1H), 7.57 (s, 1H), 7.43 (d, J = 4.0 Hz, 2H), 7.30 (d, J = 4.0 Hz, 2H), 7.23 (d, J = 4.0 Hz, 1H), 7.16~7.11 (m, 2H), 5.88 (d, J = 2.4 Hz, 1H), 3.09 (s, 3H), 2.94 (d, J = 2.4 Hz, 3H), 2.65 (s, 3H), 2.45 (s, 3H). | 619 |
| 443 | 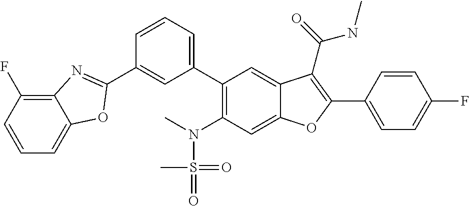 | ¹H-NMR (CDCl₃, 400 MHz) δ 8.30 (s, 1H), 8.24 (d, J = 7.6 Hz, 1H), 7.84~7.95 (m, 2H), 7.82 (s, 1H), 7.53~7.66 (m, 3H), 7.22~7.35 (m, 2H), 6.98~7.24 (m, 3H), 5.82 (s, 1H), 3.13 (s, 3H), 2.94 (d, J = 4.8 Hz, 3H), 2.63 (s, 3H). | 588 |
| 444 | 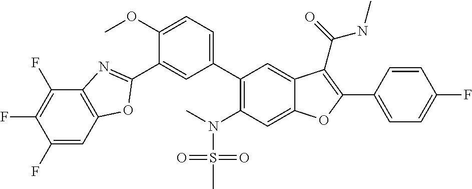 | ¹H-NMR (CDCl₃, 400 MHz) δ 8.12~8.25 (m, 1H), 7.74~7.86 (m, 3H), 7.54~7.64 (m, 2H), 6.91~7.13 (m, 4H), 5.91 (s, 1H), 4.04 (d, ~ 31.6 Hz, 3H), 3.08 (s, 3H), 2.93 (s, 3H), 2.74 (s, 3H). | 654 |
| 445 | 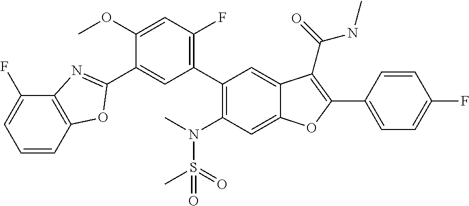 | ¹H-NMR (CDCl₃, 400 MHz) δ 8.16 (d, J = 8.4 Hz, 1H), 7.86~7.90 (m, 2H), 7.80 (s, 1H), 7.60 (s, 1H), 7.31 (d, J = 8.4 Hz, 1H), 7.20~7.25 (m, 1H), 7.13 (t, J = 8.4 Hz, 2H), 6.99 (t, J = 8.4 Hz, 1H), 6.82 (d, J = 11.2 Hz, 1H), 5.92 (d, J = 4.4 Hz, 1H), 4.00 (s, 3H), 3.21 (s, 3H), 2.94 (d, J = 4.8 Hz, 3H), 2.61 (s, 3H). | 636 |
| 446 | 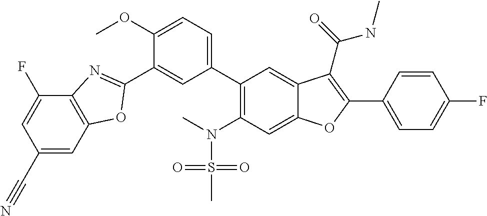 | ¹H-NMR (CDCl₃, 400 MHz) δ 8.22 (s, 1H), 7.85~7.88 (m, 2H), 7.82 (s, 1H), 7.69 (d, J = 6.8 Hz, 2H), 7.55 (s, 1H), 7.32 (d, J = 8.8 Hz, 1H), 7.13~7.17 (m, 3H), 5.84 (s, 1H), 4.02 (s, 3H), 3.09 (s, 3H), 2.93 (d, J = 4.8 Hz, 3H), 2.78 (s, 3H). | 643 |
| 447 | 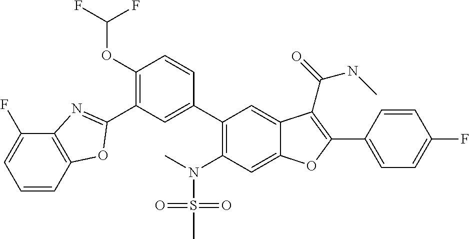 | ¹H-NMR (CDCl₃, 400 MHz) δ 8.40 (s, 1H), 7.98 (t, J = 5.6 Hz, 2H), 7.92 (s, 1H), 7.75 (d, J = 7.2 Hz, 1H), 7.65 (s, 1H), 7.46~7.51 (m, 2H), 7.36~7.41 (m, 1H), 7.23~7.29 (m, 2H), 7.14 (t, J = 8.4 Hz, 1H), 6.66~7.03 (m, 1H), 5.93 (s, 1H), 3.22 (s, 3H), 3.04 (d, J = 4.8 Hz, 3H), 2.85 (s, 3H). | 654 |
| 448 | 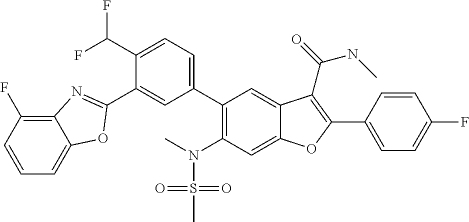 | ¹H-NMR (CDCl₃, 400 MHz) δ 8.37 (s, 1H), 7.88~8.19 (m, 5H), 7.75~7.77 (m, 1H), 7.63 (s, 1H), 7.24~7.41 (m, 2H), 7.17~7.21 (m, 2H), 7.07~7.12 (m, 1H), 5.91 (br s, 1H), 3.16 (s, 3H), 2.96 (d, J = 4.0 Hz, 3H), 2.76 (s, 3H). | 638 |
| 449 | 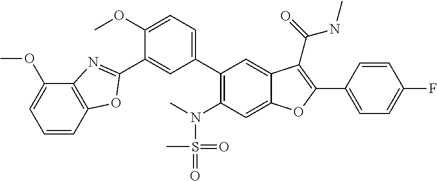 | ¹H-NMR (CDCl₃, 400 MHz) δ 8.16 (s, 1H), 7.55~7.90 (m, 5H), 7.09~7.16 (m, 5H), 5.97 (s, 1H), 3.96~3.99 (m, 6H), 3.08 (s, 3H), 2.96 (s, 3H), 2.69 (s, 3H). | 630 |
| 450 | 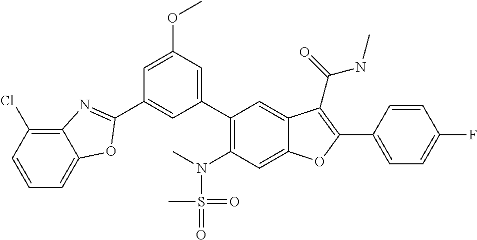 | ¹H-NMR (CDCl₃, 400 MHz) δ 7.95 (t, J = 8.4 Hz, 1H), 7.85~7.88 (m, 2H), 7.64 (s, 1H), 7.51 (d, J = 8.0 Hz, 1H), 7.39 (d, J = 8.4 Hz, 1H), 7.45 (d, J = 8.0 Hz, 1H), 7.30 (d, J = 7.6 Hz, 1H), 7.23 (d, J = 8.0 Hz, 1H), 7.09~7.15 (m, 3H), 5.91 (d, J = 4.0 Hz, 1H), 3.98 (s, 3H), 2.94 (d, J = 4.8 Hz, 3H), 2.75 (s, 3H), 2.77 (s, 3H). | 635 |
| 451 | 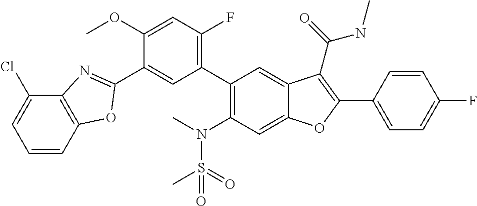 | ¹H-NMR (CDCl₃, 400 MHz) δ 8.16 (d, J = 8.4 Hz, 1H), 7.87~7.90 (m, 2H), 7.80 (s, 1H), 7.60 (s, 1H), 7.42 (d, J = 8.0 Hz, 1H), 7.28 (d, J = 8.0 Hz, 1H), 7.21 (d, J = 8.0 Hz, 1H), 7.13 (t, J = 8.4 Hz, 2H), 6.81 (d, J = 11.6 Hz, 1H), 5.94 (d, J = 3.6 Hz, 1H), 3.99 (s, 3H), 3.20 (s, 3H), 2.94 (d, J = 4.8 Hz, 3H), 2.61 (s, 3H). | 653 |
| 452 | 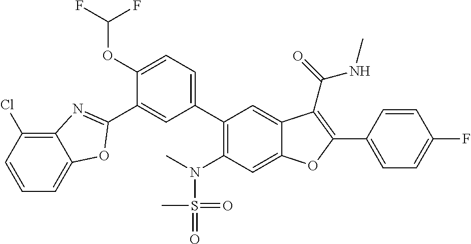 | ¹H-NMR (CDCl₃, 400 MHz) δ 8.31 (s, 1H), 7.86~7.89 (m, 2H), 7.81 (s, 1H), 7.65 (d, J = 8.4 Hz, 1H), 7.57 (s, 1H), 7.46 (d, J = 8.0 Hz, 1H), 7.38 (d, J = 8.8 Hz, 1H), 7.32 (d, J = 8.8 Hz, 1H), 7.26 (t, J = 8.0 Hz, 1H), 7.14 (t, J = 8.4 Hz, 2H), 6.56~6.93 (m, 1H), 5.91 (d, J = 3.6 Hz, 1H), 3.10 (s, 3H), 2.94 (d, J = 3.6 Hz, 3H), 2.79 (s, 3H). | 671 |
| 453 | 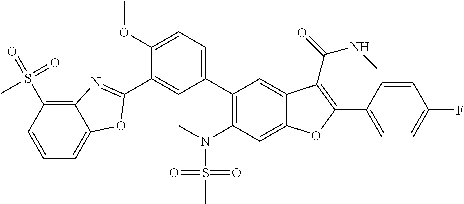 | ¹H-NMR (CDCl₃, 400 MHz) δ 8.34 (s, 1H), 7.85~7.97 (m, 5H), 7.69 (d, J = 8.0 Hz, 1H), 7.62 (s, 1H), 7.47~7.51 (m, 1H), 7.15~7.25 (m, 3H), 6.05 (br s, 1H), 4.05 (s, 3H), 3.47 (s, 3H), 3.17 (s, 3H), 2.96 (d, J = 4.8 Hz, 3H), 2.90 (s, 3H). | 678 |
| 454 | 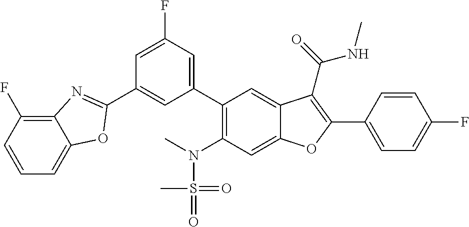 | ¹H-NMR (CDCl₃, 400 MHz) δ 8.10 (s, 1H), 7.94 (d, J = 8.4 Hz, 1H), 7.88 (t, J = 5.6 Hz, 2H), 7.83 (s, 1H), 7.59 (s, 1H), 7.35 (d, J = 8.4 Hz, 2H), 7.26~7.29 (m, 1H), 7.14 (d, J = 8.0 Hz, 2H), 7.04 (t, J = 8.8 Hz, 1H), 5.81 (s, 1H), 3.14 (s, 3H), 2.94 (d, J = 4.8 Hz, 3H), 2.73 (s, 3H). | 606 |
| 455 | 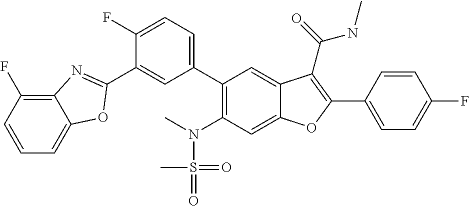 | ¹H-NMR (CDCl₃, 400 MHz) δ 8.23~8.25 (m, 1H), 7.82~7.85 (m, 2H), 7.77 (s, 1H), 7.59~7.61 (m, 1H), 7.52 (s, 1H), 7.37 (d, J = 8.0 Hz, 1H), 7.25~7.30 (m, 2H), 7.08~7.12 (m, 2H), 7.00~7.04 (m, 1H), 6.08 (s, 1H), 3.08 (s, 3H), 2.94 (d, J = 4.8 Hz, 3H), 2.76 (s, 3H). | 606 |
| 456 | 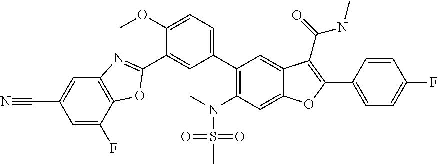 | ¹H-NMR (CDCl₃, 400 MHz) δ 8.22 (s, 1H), 7.87~7.93 (m, 3H), 7.87 (s, 1H), 7.73~7.75 (m, 1H), 7.61 (s, 1H), 7.40 (d, J = 8.8 Hz, 1H), 7.17~7.23 (m, 3H), 5.88 (s, 1H), 4.09 (s, 3H), 3.16 (s, 3H), 2.98 (d, J = 4.8 Hz, 3H), 2.81 (s, 3H). | 643 |
| 457 | 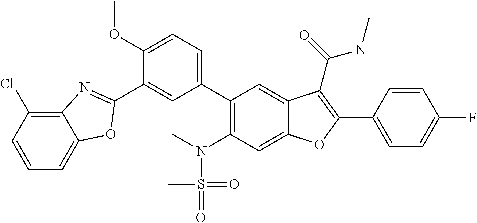 | ¹H-NMR (CDCl₃, 400 MHz) δ 8.17 (s, 1H), 7.87~8.17 (m, 2H), 7.78 (s, 1H), 7.63 (d, J = 1.6 Hz, 1H), 7.56 (s, 1H), 7.45 (d, J = 8.0 Hz, 1H), 7.30 (d, J = 7.6 Hz, 1H), 7.23 (d, J = 8.0 Hz, 1H), 7.09~7.15 (m, 3H), 5.91 (d, J = 4.8 Hz, 1H), 3.98 (s, 3H), 3.10 (s, 3H), 2.94 (d, J = 4.8 Hz, 3H), 2.75 (s, 3H). | 635 |
| 458 | 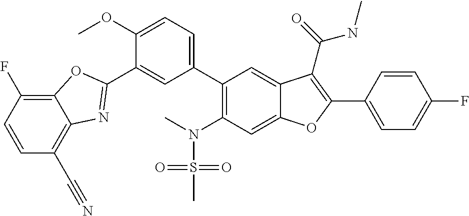 | ¹H-NMR (CDCl₃, 400 MHz) δ 8.24 (s, 1H), 7.85~7.89 (m, 2H), 7.81 (s, 1H), 7.67~7.69 (m, 1H), 7.57~7.60 (m, 2H), 7.10~7.17 (m, 4H), 5.85 (d, J = 4.0 Hz, 1H), 4.03 (s, 3H), 3.11 (s, 3H), 2.94 (d, J = 5.2 Hz, 3H), 2.79 (s, 3H). | 643 |
| 459 | 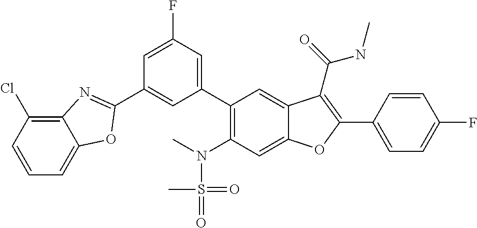 | ¹H-NMR (CDCl₃, 400 MHz) δ 8.20 (s, 1H), 8.03 (d, J = 8.8 Hz, 1H), 7.94~7.98 (m, 2H), 7.92 (s, 1H), 7.67 (s, 1H), 7.54 (d, J = 8.0 Hz, 1H), 7.41~7.45 (m, 2H), 7.33~7.37 (m, 1H), 7.21~7.25 (m, 2H), 6.02 (d, J = 4.0 Hz, 1H), 3.21 (s, 3H), 3.04 (d, J = 4.8 Hz, 3H), 2.84 (s, 3H). | 623 |
| 460 | 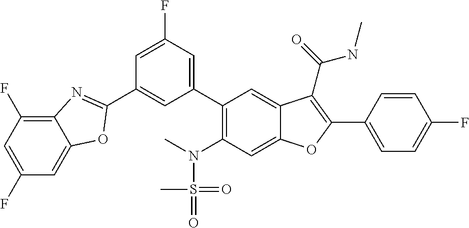 | ¹H-NMR (CDCl₃, 400 MHz) δ 8.05 (s, 1H), 7.84~7.91 (m, 4H), 7.57 (s, 1H), 7.35 (d, J = 9.2 Hz, 1H), 7.10~7.16 (m, 3H), 6.83~6.88 (m, 1H), 3.14 (s, 3H), 2.94 (d, J = 4.8 Hz, 3H), 2.74 (s, 3H). | 624 |
| 461 | 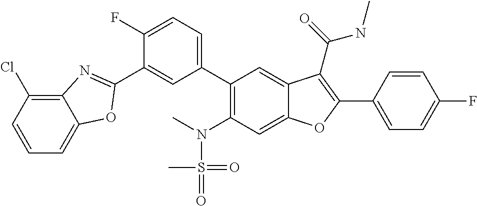 | ¹H-NMR (CDCl₃, 400 MHz) δ 8.30 (d, J = 1.0 Hz, 1H), 7.90~7.86 (m, 2H), 7.82 (s, 1H), 7.65~7.63 (m, 1H), 7.57 (s, 1H), 7.49 (d, J = 4.0 Hz, 1H), 7.17~7.12 (m, 3H), 7.15 (t, J = 8.4 Hz, 2H), 5.89~5.82 (m, 1H), 3.08 (s, 3H), 2.95 (d, J = 2.2 Hz, 3H), 2.79 (s, 3H). | 623 |
| 462 | 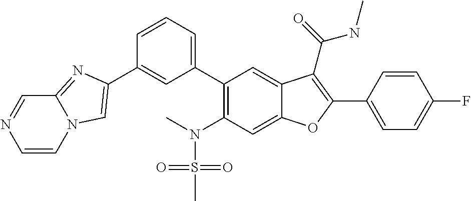 | ¹H-NMR (CDCl₃, 400 MHz) δ 9.16 (s, 1H), 8.12~8.14 (m, 3H), 7.96~8.08 (m, 1H), 7.90~7.93 (m, 3H), 7.80 (s, 1H), 7.45~7.51 (m, 3H), 7.11~7.34 (m, 2H), 6.02 (d, J = 8.0 Hz, 1H), 3.01 (s, 3H), 2.92 (d, J = 4.8 Hz, 3H), 2.77 (s, 3H). | 570 |
| 463 | 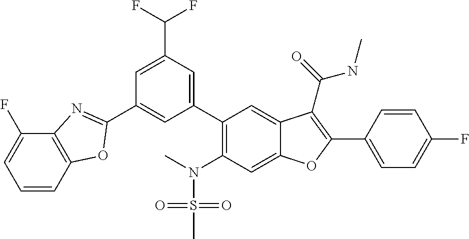 | ¹H-NMR (CDCl₃, 400 MHz) δ 8.27~8.53 (m, 2H), 8.02~8.06 (m, 3H), 7.79 (s, 1H), 7.61 (s, 1H), 7.36~7.38 (m, 1H), 7.26~7.31 (m, 1H), 7.13~7.18 (m, 2H), 7.05 (t, J = 8.8 Hz, 1H), 6.62~6.90 (m, 1H), 3.87 (s, 3H), 3.11 (s, 3H), 2.79 (s, 3H). | 638 |
| 464 | 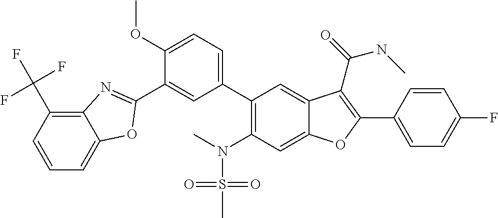 | ¹H-NMR (CDCl₃, 400 MHz) δ 8.31 (s, 1H), 7.94~7.96 (dd, J₁ = 4.0 Hz, J₂ = 8.0 Hz, 2H), 7.88 (s, 1H), 7.80~7.82 (d, J = 8.0 Hz, 1H), 7.62~7.72 (m, 3H), 7.42~7.46 (m, 1H), 7.23 (s, 1H), 7.17~7.21 (m, 2H), 5.89 (s, 1H), 4.07 (s, 3H), 3.17 (s, 3H), 3.00~3.02 (d, J = 8.0 Hz, 3H), 2.86 (s, 3H). | 668 |
| 465 | 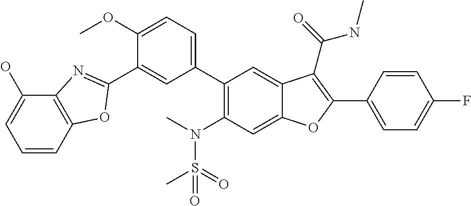 | H-NMR (CDCl₃, 400 MHz) δ 8.14 (d, J = 1.6 Hz, 1H), 7.85~7.89 (m, 2H), 7.81 (s, 1H), 7.59~7.61 (m, 1H), 7.55 (s, 1H), 7.02~7.17 (m, 5H), 6.80 (d, J = 6.8 Hz, 1H), 5.83 (d, J = 2.8 Hz, 1H), 4.00 (s, 3H), 3.09 (s, 3H), 2.93 (d, J = 4.8 Hz, 3H), 2.72 (s, 3H). | DB |
| 466 | 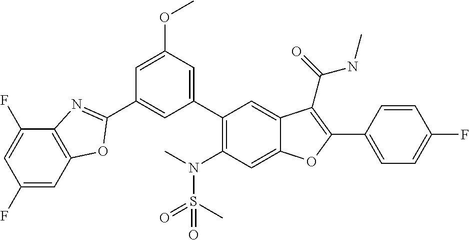 | ¹H-NMR (CDCl₃, 400 MHz) δ 7.85~7.94 (m, 4H), 7.77 (s, 1H), 7.61 (s, 1H), 7.28 (s, 1H), 7.16~7.22 (m, 3H), 6.90 (d, J = 9.6 Hz, 1H), 6.06 (s, 1H), 3.96 (s, 3H), 3.14 (s, 3H), 3.00 (d, J = 4.4 Hz, 3H), 2.80 (s, 3H). | 636 |
| 467 | 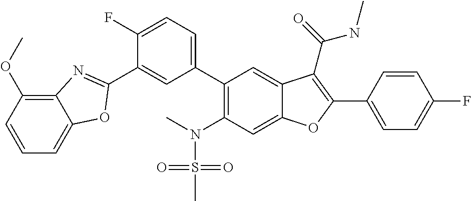 | ¹H-NMR (CDCl₃, 400 MHz) δ 8.36~8.37 (m, 1H), 7.93~7.96 (m, 2H), 7.84 (s, 1H), 7.63~7.67 (m, 1H), 7.61 (s, 1H), 7.32~7.35 (m, 2H), 7.23~7.31 (m, 1H), 7.17~7.21 (m, 2H), 6.83 (d, J = 7.6 Hz, 1H), 6.05 (d, J = 4.8 Hz, 1H), 4.05 (s, 3H), 3.14 (s, 3H), 3.02 (d, J = 4.8 Hz, 3H), 2.80 (s, 3H). | 618 |
| 468 | 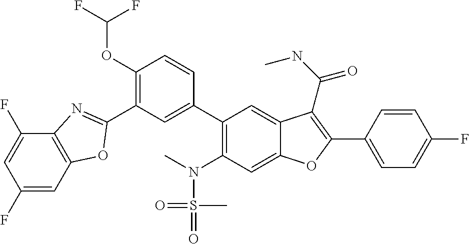 | ¹H-NMR (CDCl₃, 400 MHz) δ 8.31 (s, 1H), 7.62~7.71 (m, 2H), 7.56 (d, J = 6.0 Hz, 1H), 7.52 (s, 1H), 7.44 (d, J = 6.4 Hz, 1H), 7.37~7.40 (m, 2H), 7.19 (d, J = 9.2 Hz, 2H), 6.59~6.98 (m, 2H), 5.86 (t, J = 8.0 Hz, 1H), 3.17 (s, 3H), 2.99 (d, J = 4.4 Hz, 3H), 2.82 (s, 3H). | 672 |
| 469 | 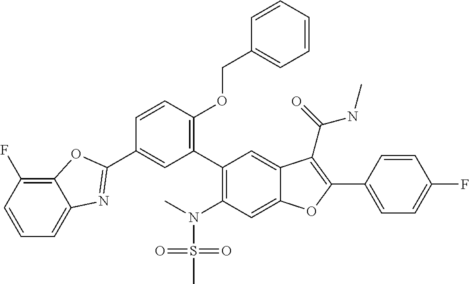 | ¹H-NMR (CDCl₃, 400 MHz) δ 8.18 (d, J = 12.0 Hz, 2H), 7.42~7.92 (m, 6H), 6.98~7.19 (m, 9H), 5.82~5.89 (m, 1H), 5.09 (s, 2H), 3.03 (d, J = 11.2 Hz, 3H), 2.91 (s, 3H), 2.47 (s, 3H). | 694 |
| 470 | 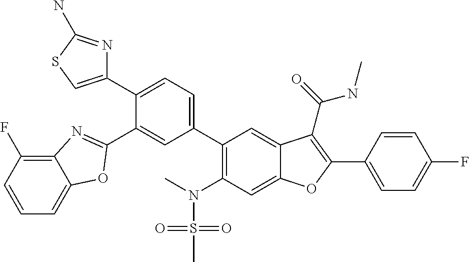 | ¹H-NMR (MeOD, 400 MHz) δ 8.40 (s, 1H), 7.90~7.98 (m, 2H), 7.80 (s, 1H), 7.81 (d, J = 8.0 Hz, 1H), 7.71~7.76 (m, 2H), 7.41~7.47 (m, 1H), 7.37~7.39 (m, 1H), 7.24~7.29 (m, 2H), 7.11~7.15 (m, 1H), 6.90 (s, 1H), 3.26 (s, 3H), 2.96 (d, J = 3.0 Hz, 3H), 2.94 (s, 3H). | 686 |
| 471 | 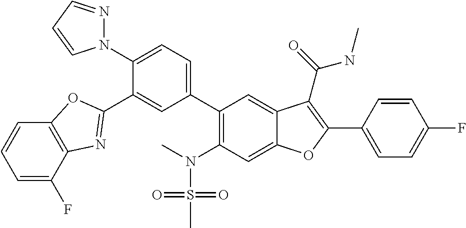 | ¹H-NMR (CDCl₃, 400 MHz) δ 8.20 (s, 1H), 7.86~7.90 (m, 2H), 7.85 (s, 1H), 7.76 (d, J = 6.4 Hz, 1H), 7.62~7.66 (m, 3H), 7.60 (d, J = 2.0 Hz, 2H), 7.09~7.22 (m, 3H), 6.98 (t, J = 8.8 Hz, 1H), 6.41 (s, 1H), 5.82 (d, J = 5.2 Hz, 1H), 3.10 (s, 3H), 2.95 (d, J = 5.2 Hz, 3H), 2.96 (s, 3H). | 654 |
| 472 | 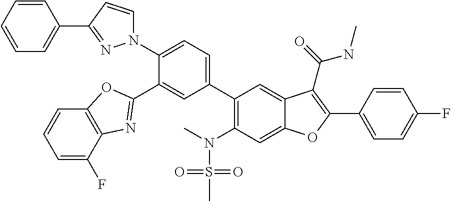 | ¹H-NMR (CDCl₃, 400 MHz) δ 8.21 (s, 1H), 7.85~7.91 (m, 2H), 7.80 (s, 1H), 7.78 (d, J = 2.0 Hz, 1H), 7.73 (d, J = 8.4 Hz, 1H), 7.68 (d, J = 2.4 Hz, 1H), 7.60 (s, 1H), 7.57 (d, J = 6.8 Hz, 2H), 7.22~7.27 (m, 3H), 7.09~7.19 (m, 4H), 6.99 (t, J = 8.8 Hz, 1H), 6.90 (d, J = 2.4 Hz, 1H), 5.83 (d, J = 4.4 Hz, 1H), 3.11 (s, 3H), 2.95 (d, J = 5.2 Hz, 3H), 2.89 (s, 3H). | 730 |
| 473 | 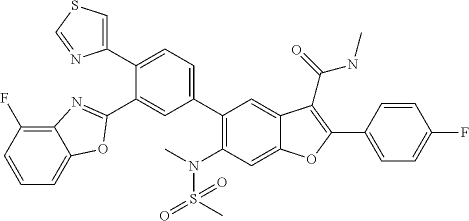 | ¹H-NMR (CDCl₃, 400 MHz) δ 8.71 (s, 1H), 8.19 (s, 1H), 7.95~7.98 (m, 2H), 7.91 (s, 1H), 7.85 (d, J = 8.4 Hz, 1H), 7.79 (d, J = 8.0 Hz, 1H), 7.68 (s, 1H), 7.48 (s, 1H), 7.21~7.26 (m, 1H), 7.11~7.19 (m, 3H), 7.06 (t, J = 8.8 Hz, 1H), 5.88 (s, 1H), 3.18 (s, 3H), 3.02 (d, J = 4.8 Hz, 3H), 2.87 (s, 3H). | 671 |
| 474 | 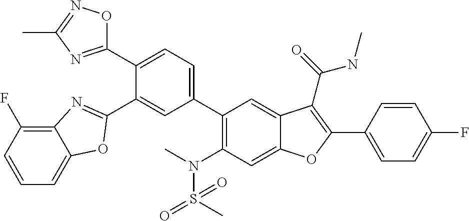 | ¹H-NMR (CDCl₃, 400 MHz) δ 8.18 (s, 1H), 7.95~7.97 (m, 1H), 7.83~7.87 (m, 3H), 7.76~7.78 (m, 1H), 7.56 (s, 1H), 7.22~7.26 (m, 2H), 7.13~7.15 (m, 2H), 7.00~7.10 (m, 1H), 6.01 (d, J = 8.0 Hz, 1H), 3.09 (s, 3H), 2.92 (d, J = 8.0 Hz, 3H), 2.80 (s, 3H), 2.37 (s, 3H). | 670 |
| 475 | 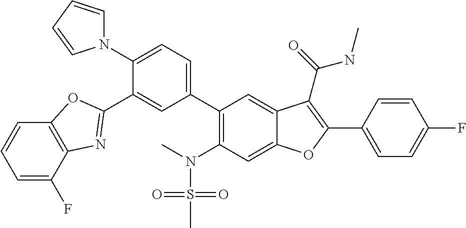 | ¹H-NMR (CDCl₃, 400 MHz) δ 8.25 (s, 1H), 7.95~7.98 (m, 2H), 7.92 (s, 1H), 7.79~7.82 (m, 1H), 7.66 (s, 1H), 7.58 (d, J = 8.4 Hz, 1H), 7.29 (t, J = 3.2 Hz, 1H), 7.19~7.24 (m, 3H), 7.04~7.09 (m, 1H), 6.82 (t, J = 2.0 Hz, 2H), 6.30 (t, J = 2.0 Hz, 2H), 5.87 (d, J = 4.8 Hz, 1H), 3.20 (s, 3H), 3.02 (d, J = 5.2 Hz, 3H), 2.91 (s, 3H). | 653 |
| 476 | 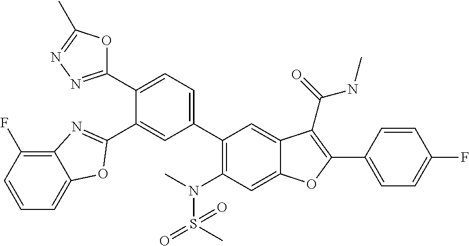 | ¹H-NMR (CDCl₃, 400 MHz) δ 8.21 (s, 1H), 7.84~7.93 (m, 4H), 7.75~7.77 (m, 1H), 7.57 (s, 1H), 7.19~7.26 (m, 2H), 7.11~7.15 (m, 2H), 6.99~7.04 (m, 1H), 6.05 (d, J = 4.4 Hz, 1H), 3.09 (s, 3H), 2.92 (d, J = 5.2 Hz, 3H), 2.80 (s, 3H), 2.44 (s, 3H). | 670 |
| 477 | 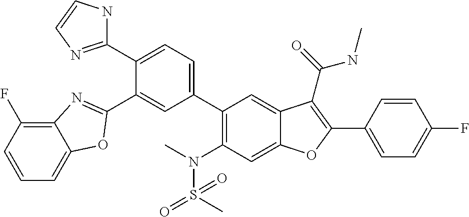 | ¹H-NMR (MeOD, 400 MHz) δ 8.76 (s, 1H), 8.15~8.20 (m, 4H), 8.09 (d, J = 8.0 Hz, 1H), 8.02 (s, 1H), 7.91 (s, 2H), 7.60~7.70 (m, 2H), 7.47~7.52 (m, 2H), 7.32~7.37 (m, 1H), 3.53 (s, 3H), 3.29 (s, 3H), 3.15 (s, 3H). | 654 |
| 478 | 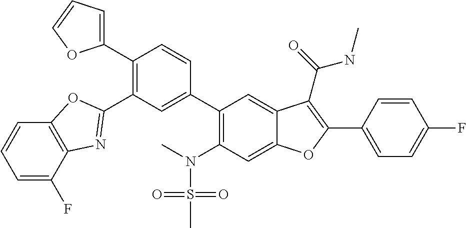 | ¹H-NMR (CDCl₃, 400 MHz) δ 7.88 (s, 1H), 7.80~7.82 (m, 2H), 7.70~7.78 (m, 2H), 7.68 (s, 1H), 7.59 (s, 1H), 7.34 (s, 1H), 7.25 (t, J = 6.8 Hz, 2H), 7.01~7.16 (m, 3H), 6.40 (s, 2H), 5.81 (d, J = 4.4 Hz, 1H), 3.11 (s, 3H), 2.95 (d, J = 8.8 Hz, 3H), 2.77 (s, 3H). | 654 |
| 479 | 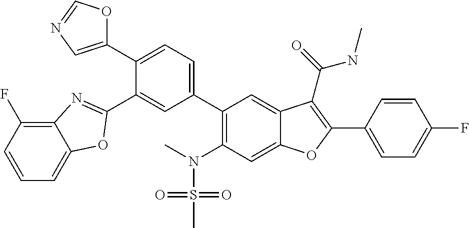 | ¹H-NMR (CDCl₃, 400 MHz) δ 8.10 (s, 1H), 7.83~7.88 (m, 4H), 7.31~7.74 (m, 2H), 7.56 (s, 1H), 7.23~7.27 (m, 3H), 7.10~7.19 (m, 2H), 7.01~7.05 (m, 1H), 5.93 (d, J = 4.0 Hz, 1H), 3.10 (s, 3H), 2.93 (d, J = 4.8 Hz, 3H), 2.80 (s, 3H). | 655 |
| 480 | 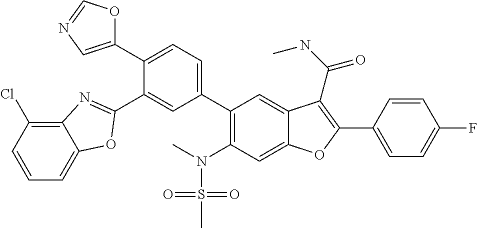 | ¹H-NMR (CDCl₃, 400 MHz) δ 8.18 (s, 1H), 7.89~7.94 (m, 4H), 7.77~7.83 (m, 2H), 7.63 (s, 1H), 7.39 (t, J = 8.0 Hz, 2H), 7.27~7.32 (m, 2H), 7.19 (t, J = 8.4 Hz, 2H), 5.92 (d, J = 4.0 Hz, 1H), 3.14 (s, 3H), 2.99 (d, J = 4.8 Hz, 3H), 2.89 (s, 3H). | 672 |
| 481 | 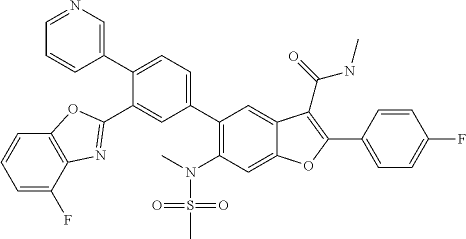 | ¹H-NMR (CDCl₃, 400 MHz) δ 8.91 (s, 1H), 8.85 (s, 1H), 8.40 (s, 1H), 7.27 (d, J = 8.0 Hz, 1H), 7.91 (s, 1H), 7.86~7.89 (m, 2H), 7.78~7.80 (m, 2H), 7.60 (s, 1H), 7.46 (d, J = 8.0 Hz, 1H), 7.22~7.25 (m, 1H), 7.14~7.19 (m, 3H), 6.97 (t, J = 8.4 Hz, 1H), 5.85 (d, J = 4.4 Hz, 1H), 3.16 (s, 3H), 2.94 (d, J = 4.8 Hz, 3H), 2.87 (s, 3H). | 665 |
| 482 | 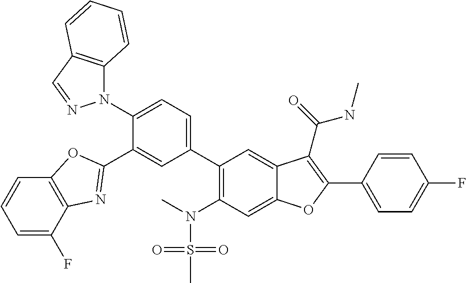 | ¹H-NMR (CDCl₃, 400 MHz) δ 8.41 (s, 1H), 8.19 (s, 1H), 7.83~7.90 (m, 4H), 7.74~7.79 (m, 2H), 7.63 (s, 1H), 7.32 (d, J = 4.0 Hz, 2H), 7.11~7.17 (m, 3H), 7.08 (t, J = 4.4 Hz, 1H), 6.92 (t, J = 8.8 Hz, 1H), 6.54 (t, J = 8.0 Hz, 1H), 6.02 (s, 1H), 3.16 (s, 3H), 2.97 (d, J = 4.8 Hz, 3H), 2.87 (s, 3H). | 704 |
| 483 | 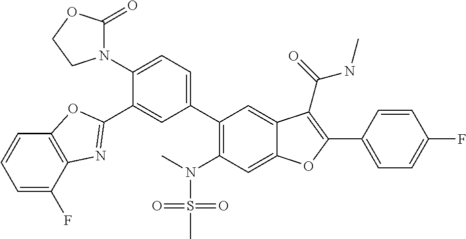 | ¹H-NMR (CDCl₃, 400 MHz) δ 8.31 (s, 1H), 7.89~7.98 (m, 2H), 7.81 (s, 1H), 7.69 (dd, J = 8.4 Hz, 1H), 7.58 (s, 1H), 7.50 (d, J = 8.0 Hz, 1H), 7.48 (s, 1H), 7.28~7.33 (m, 1H), 7.22 (t, J = 8.8 Hz, 2H), 7.02 (t, J = 8.8 Hz, 1H), 5.82 (d, J = 4.4 Hz, 1H), 4.66 (t, J = 8.4 Hz, 2H), 4.18 (t, J = 8.0 Hz, 2H), 3.13 (s, 3H), 2.95 (d, J = 5.2 Hz, 3H), 2.77 (s, 3H). | 673 |
| 484 | 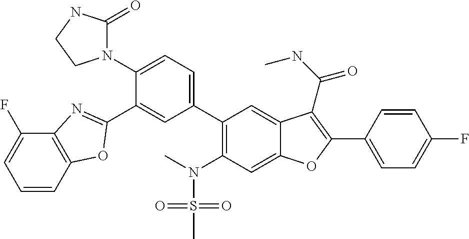 | ¹H-NMR (CDCl₃, 400 MHz) δ 8.22 (s, 1H), 7.92 (t, J = 4.4 Hz, 2H), 7.76 (s, 1H), 7.64 (d, J = 8.4 Hz, 1H), 7.56 (s, 1H), 7.41 (d, J = 8.4 Hz, 1H), 7.30 (d, J = 8.0 Hz, 1H), 7.21~7.26 (m, 1H), 7.12 (t, J = 8.4 Hz, 2H), 7.00 (t, J = 8.8 Hz, 1H), 5.95 (d, J = 4.4 Hz, 1H), 4.06 (t, J = 8.0 Hz, 2H), 3.70 (t, J = 8.0 Hz, 2H), 3.10 (s, 3H), 2.95 (d, J = 4.8 Hz, 3H), 2.77 (s, 3H). | 672 |
| 485 | 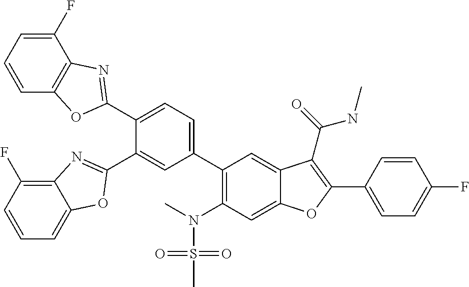 | H-NMR (CDCl₃, 400 MHz) 68.10~8.13 (m, 2H), 7.80~7.83 (m, 3H), 7.73~7.76 (m, 1H), 7.45 (s, 1H), 7.20~7.25 (m, 2H), 7.11~7.16 (m, 2H), 7.00~7.07 (m, 4H), 6.36 (d, J = 4.0 Hz, 1H), 3.08 (s, 3H), 2.95 (d, J = 4.8 Hz, 3H), 2.77 (s, 3H). | 723 |
| 486 | 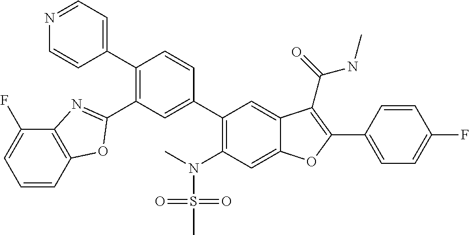 | ¹H-NMR (CDCl₃, 400 MHz) δ 8.82 (s, 2H), 8.38 (s, 1H), 7.94 (s, 1H), 7.83~7.88 (m, 4H), 7.80 (s, 1H), 7.59 (s, 1H), 7.48 (d, J = 7.6 Hz, 1H), 7.23~7.28 (m, 4H), 6.99~7.18 (m, 1H), 5.88~5.83 (m, 1H), 3.18 (s, 3H), 2.94 (d, J = 4.8 Hz, 3H), 2.90 (s, 3H). | 665 |
| 487 | 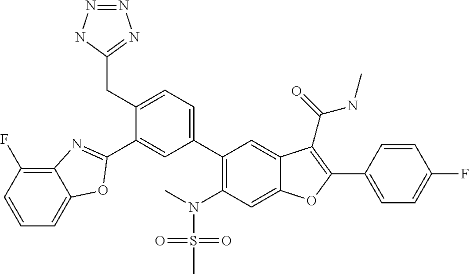 | ¹H-NMR (DMSO, 400 MHz) δ 8.54 (d, J = 8.0 Hz, 1H), 8.25 (s, 1H), 7.97~8.04 (m, 3H), 7.60~7.69 (m, 2H), 7.51 (d, J = 8.0 Hz, 1H), 7.36~7.43 (m, 3H), 7.23~7.27 (m, 1H), 4.69 (s, 2H), 3.13 (s, 3H), 2.95 (s, 3H), 2.78 (d, J = 4.0 Hz, 3H). | 670 |
| 488 | 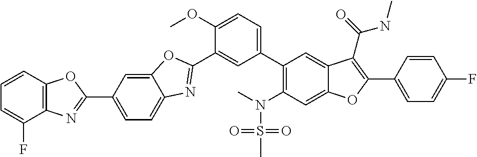 | ¹H-NMR (CDCl₃, 400 MHz) δ 8.46 (s, 1H), 8.27 (d, J = 8.0 Hz, 1H), 8.19 (s, 1H), 7.85~7.90 (m, 3H), 7.81 (s, 1H), 7.65~7.67 (m, 1H), 7.56 (s, 1H), 7.35 (d, J = 8.4 Hz, 1H), 7.23~7.28 (m, 1H), 7.12~7.16 (m, 3H), 7.01~7.06 (m, 1H), 5.96 (d, J = 3.6 Hz, 1H), 4.03 (s, 3H), 3.12 (s, 3H), 2.95 (d, J = 4.8 Hz, 3H), 2.75 (s, 3H). | 735 |
| 489 | 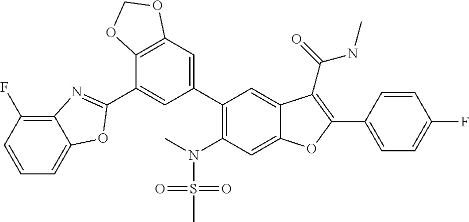 | ¹H-NMR (CDCl₃, 400 MHz) δ 7.92 (dd, J₁ = 5.2 Hz, J₂ = 8.8 Hz, 2H), 7.83 (s, 1H), 7.72 (d, J =1.6 Hz, 1H), 7.60 (s, 1H), 7.40 (d, J = 8.0 Hz, 1H), 7.26~7.33 (m, 1H), 7.17 (t, J = 8.4 Hz, 2H), 7.14 (d, J = 1.2 Hz, 1H), 7.08 (t, J = 8.8 Hz, 1H), 6.28 (s, 2H), 5.94 (d, J = 4.4 Hz, 1H), 3.16 (s, 3H), 3.00 (d, J = 5.2 Hz, 3H), 2.83 (s, 3H). | 632 |
| 490 | 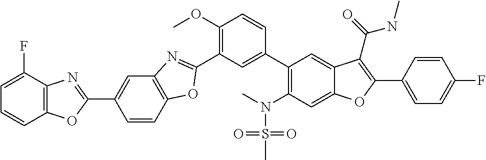 | ¹H-NMR (CDCl₃, 400 MHz) δ 8.63 (s, 1H), 8.33 (d, J = 8.0 Hz, 1H), 8.18 (s, 1H), 7.87~7.90 (m, 2H), 7.82 (s, 1H), 7.65~7.68 (m, 2H), 7.57 (s, 1H), 7.37 (d, J = 8.4 Hz, 1H), 7.25~7.28 (m, 3H), 7.01~7.15 (m, 2H), 5.80 (d, J = 5.6 Hz, 1H), 4.05 (s, 3H), 3.12 (s, 3H), 2.94 (d, J = 4.8 Hz, 3H), 2.74 (s, 3H). | 735 |
| 491 | 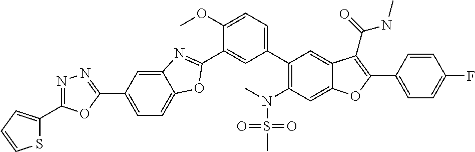 | ¹H-NMR (CDCl₃, 400 MHz) δ 8.18 (s, 1H), 8.15 (s, 1H), 8.13 (d, J = 1.2 Hz, 1H), 7.87 (dd, J₁ = 5.2 Hz, J₂ = 8.8 Hz, 2H), 7.82 (s, 1H), 7.80 (d, J = 2.8 Hz, 1H), 7.64~7.68 (s, 3H), 7.52~7.56 (s, 1H), 7.13~7.17 (m, 4H), 5.83 (d, J = 4.0 Hz, 1H), 4.04 (s, 3H), 3.11 (s, 3H), 2.94 (d, J = 4.8 Hz, 3H), 2.74 (s, 3H). | 750 |
| 492 | 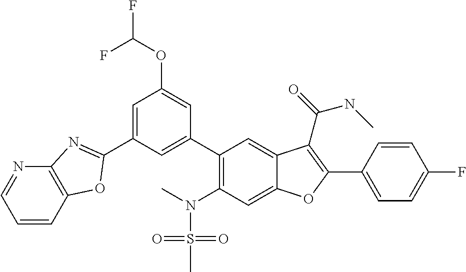 | ¹H-NMR (CDCl₃, 400 MHz) δ 8.65 (s, 1H), 8.27 (s, 1H), 8.10 (s, 1H), 7.93~7.98 (m, 4H), 7.64 (s, 1H), 7.54 (s, 1H), 7.41 (s, 1H), 7.24 (s, 1H), 7.22 (s, 1H), 6.55~6.92 (m, 1H), 5.99 (s, 1H), 3.19 (s, 3H), 3.02 (d, J = 4.8 Hz, 3H), 2.83 (s, 3H). | 637 |
| 493 | 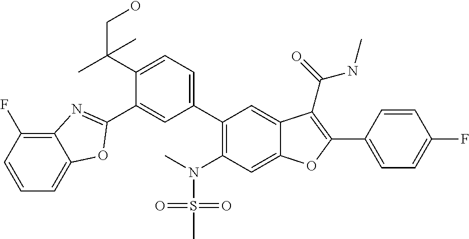 | ¹H-NMR (CDCl₃, 400 MHz) δ 8.38 (s, 1H), 7.89~7.95 (m, 4H), 7.59~7.61 (m, 1H), 7.51 (s, 1H), 7.22~7.17 (m, 1H), 6.88~6.90 (s, 3H), 6.69~6.74 (m, 1H), 6.63 (s, 1H), 3.43 (s, 2H), 3.12 (s, 3H), 2.94~2.97 (m, 6H), 1.41 (s, 6H). | 660 |
| 494 | 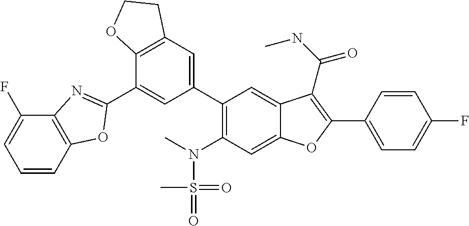 | ¹H-NMR (CDCl₃, 400 MHz) δ 8.00 (d, J = 1.2 Hz, 1H), 7.87~7.90 (m, 2H), 7.81 (s, 1H), 7.55 (s, 1H), 7.48 (s, 1H), 7.37 (d, J = 8.0 Hz, 1H), 7.24~7.26 (m, 1H), 7.11~7.16 (m, 2H), 7.03~7.05 (m, 1H), 6.20 (d, J = 4.4 Hz, 1H), 4.85~4.89 (m, 2H), 3.30~3.34 (m, 2H), 3.11 (s, 3H), 2.99 (d, J = 4.8 Hz, 3H), 2.80 (s, 3H). | 630 |
| 495 | 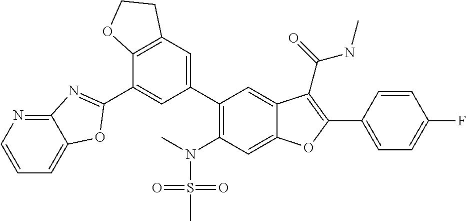 | ¹H-NMR (CDCl₃, 400 MHz) δ 8.46 (d, J = 4.0 Hz, 1H), 8.02 (s, 1H), 7.77~7.88 (m, 4H), 7.52 (s, 1H), 7.47 (s, 1H), 7.19~7.20 (m, 1H), 7.09~7.13 (m, 2H), 6.12 (d, J = 4.4 Hz, 1H), 4.819 4.84 (m, 2H), 3.27~3.31 (m, 2H), 3.09 (s, 3H), 2.94 (d, J = 4.8 Hz, 3H), 2.75 (s, 3H). | 613 |
| 496 | 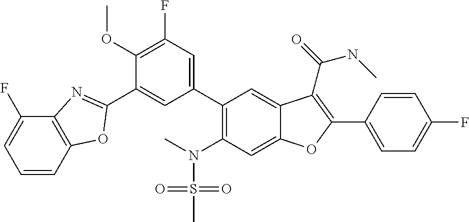 | ¹H-NMR (CDCl₃, 400 MHz) δ 7.92 (s, 1H), 7.86~7.89 (m, 2H), 7.79 (s, 1H), 7.56 (s, 1H), 7.36~7.47 (m, 2H), 7.25~7.30 (m, 1H), 7.14 (t, J = 8.4 Hz, 2H), 7.03 (t, J = 8.4 Hz, 1H), 5.87 (s, 1H), 4.11 (s, 3H), 3.12 (s, 3H), 2.95 (d, J = 3.6 Hz, 3H), 2.80 (s, 3H). | 636 |
| 497 | 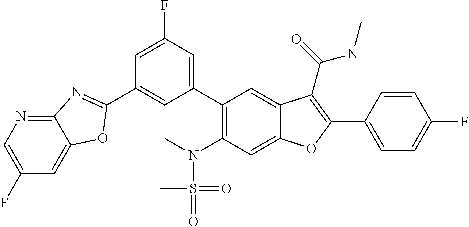 | ¹H-NMR (CDCl₃, 400 MHz) δ 8.61 (s, 1H), 8.53~8.54 (m, 1H), 8.40~8.43 (m, 1H), 8.09~8.10 (m, 2H), 7.98~8.10 (m, 3H), 7.71 (s, 1H), 7.61~7.64 (m, 1H), 7.38~7.42 (m, 2H), 3.18 (s, 3H), 3.00 (s, 3H), 2.79 (d, J = 4.4 Hz, 3H). | 606 |
| 498 | 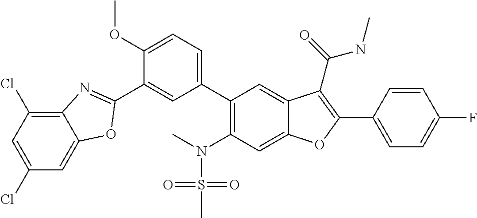 | H-NMR (CDCl₃, 400 MHz) δ 8.16 (s, 1H), 7.87~7.90 (m, 2H), 7.78 (s, 1H), 7.64 (d, J = 8.2 Hz, 1H), 7.56 (s, 1H), 7.47 (d, J = 1.6 Hz, 1H), 7.32 (d, J = 1.6 Hz, 1H), 7.09~7.16 (m, 3H), 5.91 (d, J = 4.0 Hz, 1H), 3.99 (s, 3H), 2.94 (d, J = 4.8 Hz, 3H), 2.93 (s, 3H), 2.76 (s, 3H). | 669 |
| 499 | 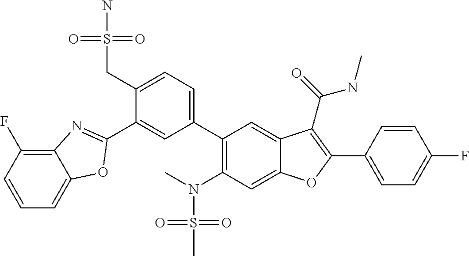 | ¹H-NMR (CDCl₃, 400 MHz) δ 8.19 (s, 1H), 7.93~7.97 (m, 2H), 7.71~7.85 (m, 3H), 7.59 (d, J = 8.0 Hz, 1H), 7.26 (t, J = 12.0 Hz, 2H), 7.14~7.20 (m, 1H), 6.66~6.74 (m, 3H), 4.50 (s, 2H), 3.15 (s, 3H), 2.91 (d, J = 4.0 Hz, 3H), 2.85 (s, 3H). | 681 |
| 500 | 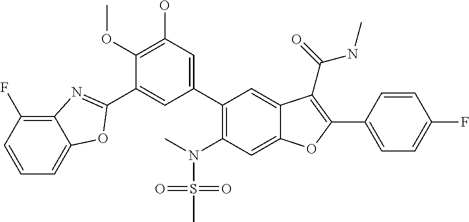 | ¹H-NMR (CDCl₃, 400 MHz) 7.94~7.98 (m, 2H), 7.91 (s, 1H), 7.71 (d, J = 2.4 Hz, 1H), 7.59 (s, 1H), 7.42~7.43 (m, 1H), 7.33~7.37 (m, 2H), 7.21~7.25 (m, 2H), 7.14 (d, J= 8.8 Hz, 1H), 5.87 (d, J = 4.4 Hz, 1H), 4.02 (s, 3H), 3.12 (s, 3H), 3.01 (d, J = 4.8 Hz, 3H), 2.92 (s, 3H). | 734 |
| 501 | 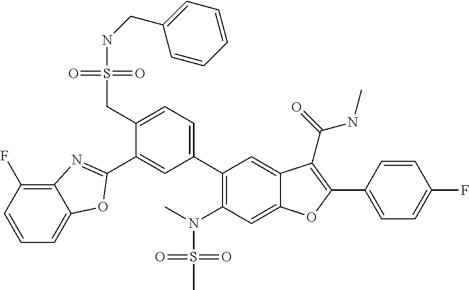 | ¹H-NMR (CDCl₃, 400 MHz) δ 8.36 (s, 1H), 7.92~7.94 (m, 2H), 7.89 (s, 1H), 7.71 (d, J = 8.0 Hz, 1H), 7.65 (dd, J = 8.0, 2.0 Hz, 1H), 7.62 (s, 1H), 7.39 (d, J = 8.0 Hz, 1H), 7.30~7.36 (m, 1H), 7.18~7.27 (m, 7H), 7.09 (t, J = 8.4 Hz, 1H), 5.96 (br s, 1H), 5.36 (s, 2H), 4.98 (t, J = 6.0 Hz, 1H), 4.25 (d, J = 6.0 Hz, 2H), 3.19 (s, 3H), 2.99 (d, J = 4.8 Hz, 3H), 2.70 (s, 3H). | 771 |
| 502 | 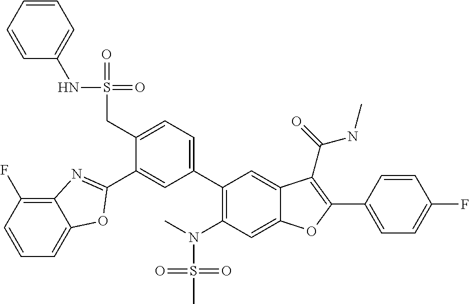 | ¹H-NMR (CDCl₃, 400 MHz) δ 8.19 (s, 1H), 7.86~7.90 (m, 2H), 7.55~7.61 (m, 3H), 7.26 (d, J = 4.0 Hz, 2H), 7.13~7.17 (m, 2H), 7.01~7.07 (m, 5H), 6.91 (s, 1H), 7.84 (t, J = 8.0 Hz, 1H), 6.70 (s, 1H), 5.84 (s, 1H), 5.42 (s, 2H), 3.13 (s, 3H), 2.94 (d, J = 8.0 Hz, 3H), 2.65 (s, 3H). | 757 |
| 503 | 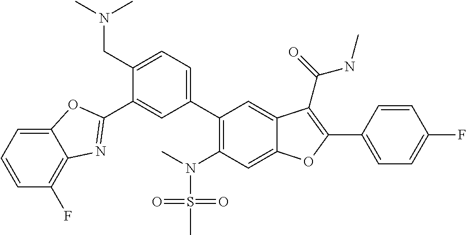 | ¹H-NMR (CDCl₃, 400 MHz) δ 8.50 (s, 1H), 7.92~8.03 (m, 3H), 7.75~7.83 (m, 3H), 7.56 (d, J = 8.0 Hz, 1H), 7.44~7.49 (m, 1H), 7.20~7.27 (m, 3H), 4.81 (s, 2H), 3.27 (d, J = 11.6 Hz, 3H), 3.08 (s, 6H), 2.96 (s, 3H), 2.91 (s, 3H). | 645 |
| 504 | 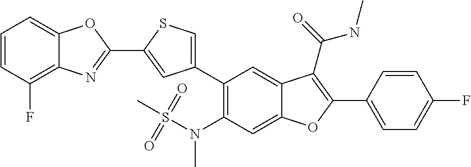 | ¹H-NMR (CDCl₃, 400 MHz) δ 8.12 (s, 1H), 7.94~7.99 (m, 3H), 7.76 (s, 1H), 7.64 (s, 1H), 7.41 (d, J = 8.4 Hz, 1H), 7.33~7.35 (m, 1H), 7.22~7.26 (m, 2H), 7.09~7.14 (m, 1H), 5.96 (d, J = 4.0 Hz, 1H), 3.26 (s, 3H), 3.05 (d, J = 4.8 Hz, 3H), 2.90 (s, 3H). | 594 |
| 505 | 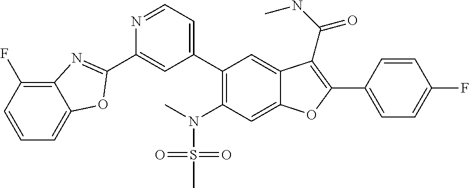 | ¹H-NMR (CDCl₃, 400 MHz) δ 8.80 (d, J = 5.2 Hz, 1H), 8.43 (d, J = 0.8 Hz, 1H), 7.85~7.88 (m, 3H), 7.56~7.58 (m, 2H), 7.42 (d, J = 8.0 Hz, 1H), 7.28~7.34 (m, 1H), 7.03~7.16 (m, 2H), 6.01 (d, J = 4.0 Hz, 1H), 5.95 (s, 1H), 3.16 (s, 3H), 2.97 (d, J = 4.0 Hz, 3H), 2.79 (s, 3H). | 589 |
| 506 | 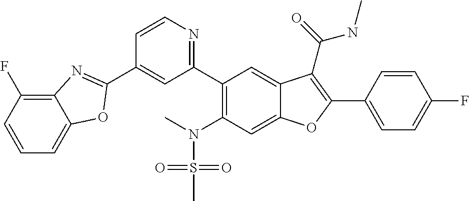 | ¹H-NMR (CDCl₃, 400 MHz) δ 8.83~8.85 (m, 1H), 8.34 (t, J = 0.8 Hz, 1H), 8.06~8.08 (m, 1H), 8.01 (s, 1H), 7.91~7.95 (m, 2H), 7.66 (s, 1H), 7.38~7.41 (m, 1H), 7.31~7.36 (m, 1H), 7.15 (t, J = 8.0 Hz, 2H), 7.05~7.10 (m, 1H), 5.92 (s, 1H), 3.22 (s, 3H), 2.95 (d, J = 4.8 Hz, 3H), 2.75 (s, 3H). | 589 |
| 507 | 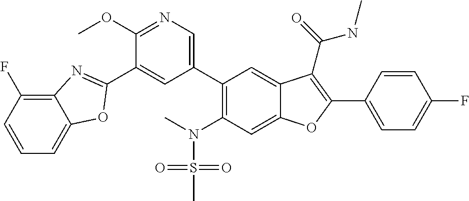 | ¹H-NMR (CDCl₃, 400 MHz) δ 8.57 (d, J = 2.0 Hz, 1H), 8.41 (d, J = 1.6 Hz, 1H), 7.90~7.93 (m, 2H), 7.86 (s, 1H), 7.63 (s, 1H), 7.40~7.42 (m, 1H), 7.30~7.34 (m, 1H), 7.18 (t, J = 4.8 Hz, 2H), 7.05~7.09 (m, 1H), 5.98 (d, J = 3.2 Hz, 1H), 4.20 (s, 3H), 3.20 (s, 3H), 3.00 (d, J = 4.4 Hz, 3H), 2.83 (s, 3H). | 619 |
| 508 | 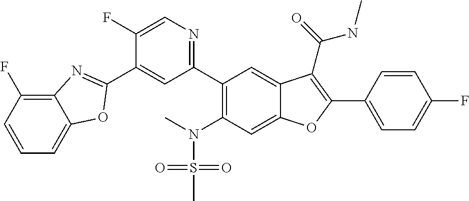 | ¹H-NMR (400 MHz, DMSO) δ 8.96 (d, J = 1.6 Hz, 1H), 8.55 (d, J = 4.8 Hz, 1H), 8.36 (d, J = 5.6 Hz, 1H), 8.06 (s, 1H), 7.96~8.00 (m, 2H), 7.88 (s, 1H), 7.74~7.76 (m, 1H), 7.52~7.57 (m, 1H), 7.34~7.41 (m, 3H), 3.26 (s, 3H), 2.97 (s, 3H), 2.80 (d, J = 4.4 Hz, 3H). | 607 |
| 509 | 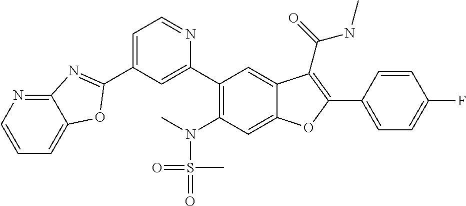 | ¹H-NMR (CDCl₃, 400 MHz) δ 8.95 (s, 1H), 8.52~8.65 (m, 2H), 8.37 (s, 1H), 8.32 (d, J = 8.4 Hz, 1H), 8.12 (d, J = 5.2 Hz, 1H), 8.08 (s, 1H), 7.98 (t, J = 3.2 Hz, 2H), 7.90 (s, 1H), 7.54 (m, 1H), 7.40 (t, J = 8.8 Hz, 2H), 3.28 (s, 3H), 2.96 (s, 3H), 2.80 (d, J = 4.4 Hz, 2H). | 572 |
| 510 | 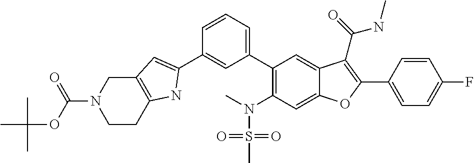 | ¹H-NMR (CDCl₃, 400 MHz) δ 8.80 (s, 1H), 7.93~7.96 (m, 2H), 7.87 (s, 1H), 7.71 (s, 1H), 7.49~7.53 (m, 2H), 7.42 (t, J = 7.6 Hz, 1H), 7.19~7.25 (m, 3H), 6.32 (d, J = 2.0 Hz, 1H), 5.93 (d, J = 4.8 Hz, 1H), 4.46 (s, 2H), 3.75 (s, 2H), 3.01 (d, J = 4.8 Hz, 3H), 2.96 (s, 3H), 2.93 (s, 3H), 2.74 (t, J = 5.6 Hz, 2H), 1.49 (s, 9H). | |
| 511 | 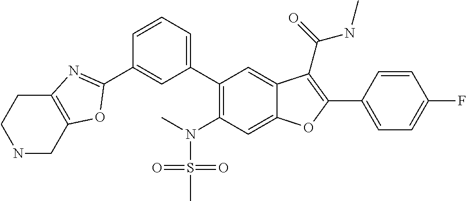 | ¹H-NMR (DMSO, 300 MHz): δ 9.34 (br, 2H), 8.53~8.48 (m, 1H), 8.03~7.95 (m, 5H), 7.62 (s, 1H), 7.60 (s, 2H), 7.41 (t, J = 8.4 Hz, 2H), 4.44 (s, 2H), 3.48~3.44 (m, 2H), 3.12 (s, 3H), 2.93 (s, 3H), 2.87~2.84 (m, 2H), 2.80 (d, J = 4.5 Hz, 3H). | 575 |
| 512 | 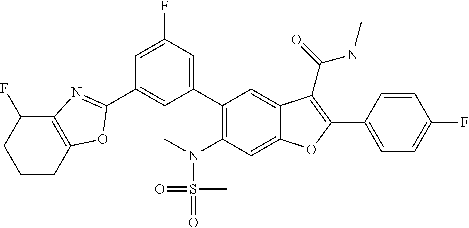 | ¹H-NMR (CDCl₃, 400 MHz) δ 7.93~7.97 (m, 2H), 7.92 (s, 1H), 7.86 (s, 1H), 7.77 (d, J = 8.0 Hz, 1H) 7.64 (s, 1H), 7.30 (s, 1H), 7.20~7.24 (m, 2H), 5.84 (s, 1H), 5.56 (d, J = 5.2 Hz, 1H), 3.18 (s, 3H), 3.01 (d, J = 8.0 Hz, 3H), 2.87~2.91 (m, 1H), 2.84 (s, 3H), 2.74 (s, 1H), 2.28~2.34 (m, 1H), 2.12~2.15 (m, 1H), 1.87~2.12 (m, 2H). | 610 |
| 513 | 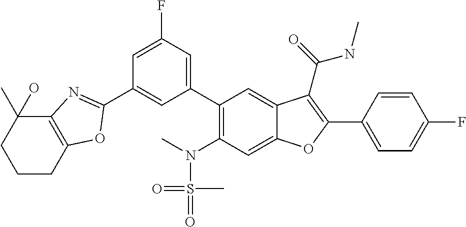 | ¹H-NMR (CDCl₃, 400 MHz) δ 7.94~7.98 (m, 3H), 7.84~7.86 (m, 2H), 7.60~7.63 (m, 2H), 7.37~7.38 (m, 1H), 7.20~7.24 (m, 2H), 5.85 (s, 1H), 3.09 (s, 3H), 3.01~3.20 (m, 3H), 2.91 (s, 3H), 2.51~2.58 (m, 4H), 2.03~2.07 (m, 2H), 1.99 (s, 3H). | 622 |
| 514 | 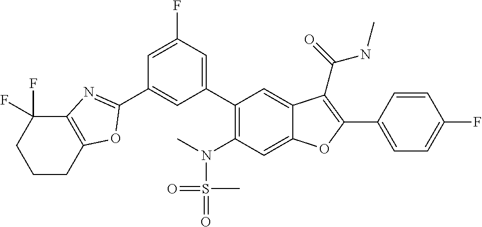 | ¹H-NMR (CDCl₃, 400 MHz): δ 7.93~7.96 (m, 3H), 7.86 (s, 1H), 7.79~7.81 (d, J = 6.0 Hz, 1H), 7.64 (s, 1H), 7.31~7.33 (d, J = 6.0 Hz, 1H), 7.20~7.24 (m, 2H), 5.86 (s, 1H), 3.18 (s, 3H), 3.02 (s, 3H), 2.84 (m, 2H), 2.76 (s, 3H), 2.27~2.34 (m, 2H), 2.15~2.17 (m, 2H). | 628 |
| 515 | 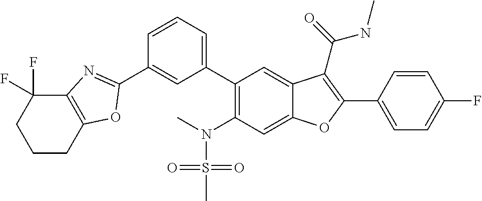 | ¹H-NMR (CDCl₃, 400 MHz): δ 8.13~8.08 (m, 2H), 7.98~7.93 (m, 2H), 7.84 (s, 1H), 7.63 (s, 1H), 7.60~7.52 (m, 2H), 7.23~7.18 (m, 2H), 5.87 (br, 1H), 3.16 (s, 3H), 3.00 (d, J = 6.4 Hz, 3H), 2.84~2.80 (m, 2H), 2.64 (s, 3H), 2.38~2.24 (m, 2H), 2.18~2.11 (m, 2H). | 610 |
| 516 | 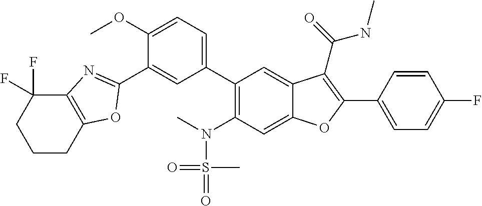 | ¹H-NMR (CDCl₃, 400 MHz): δ 7.99 (s, 1H), 7.90~7.94 (m, 2H), 7.78 (s, 1H), 7.58~7.61 (m, 2H), 7.08~7.22 (m, 3H), 6.16 (br, 1H), 3.99 (s, 3H), 3.12 (s, 3H), 3.02 (d, J = 4.4 Hz, 3H), 2.80~2.83 (m, 2H), 2.76 (s, 3H), 2.28~2.32 (m, 2H), 2.12~2.14 (m, 2H). | 640 |
| 517 | 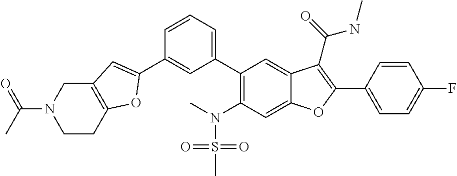 | ¹H-NMR (CDCl₃, 400 MHz) δ 7.93~7.96 (m, 2H), 7.84 (d, J = 6.4 Hz, 1H), 7.74 (d, J = 10.0 Hz, 1H), 7.62~7.72 (m, 2H), 7.44~7.48 (m, 1H), 7.34 (d, J = 7.2 Hz, 1H), 7.21 (t, J = 8.4 Hz, 2H), 6.58 (s, 1H), 5.88 (s, 1H), 4.58 (s, 1H), 4.44 (s, 1H), 3.98 (t, J = 5.6 Hz, 1H), 3.80 (t, J = 5.6 Hz, 1H), 3.14 (d, J = 6.0 Hz, 3H), 3.00 (d, J = 4.8 Hz, 3H), 2.80~2.88 (m, 2H), 2.65 (d, J = 16.8 Hz, 3H), 2.23 (d, J = 8.4 Hz, 3H). | 616 |
| 518 | 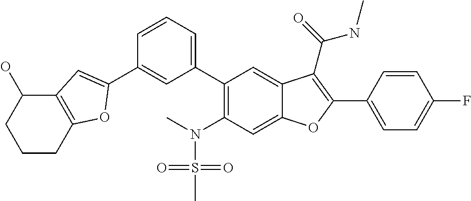 | ¹H-NMR (CDCl₃, 400 MHz) δ 7.94~7.98 (m, 2H), 7.82 (s, 1H), 7.63~7.15 (m, 3H), 6.73~7.47 (m, 4H), 6.73 (s, 1H), 5.87 (d, J = 3.2 Hz, 1H), 4.80 (s, 1H), 3.17 (s, 3H), 3.00 (d, J = 6.2 Hz, 3H), 2.67~2.75 (m, 1H), 2.59 (s, 3H), 1.84~2.05 (m, 6H). | 589 |
| 519 | 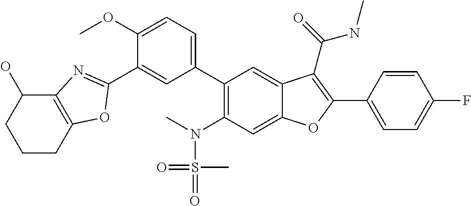 | ¹H-NMR (CDCl₃, 400 MHz) δ 8.07 (s, 1H), 7.89~7.94 (m, 3H), 7.76 (d, J = 8.8 Hz, 1H), 7.59 (s, 1H), 7.71~7.25 (m, 3H), 5.93 (s, 1H), 5.05 (s, 1H), 4.05~4.11 (m, 4H), 3.12 (s, 3H), 2.98 (d, J = 8.8 Hz, 3H), 2.69~2.86 (m, 5H), 1.98~2.19 (m, 4H). | 620 |
| 520 | 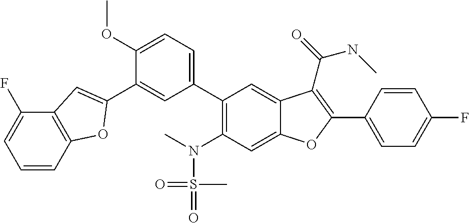 | ¹H-NMR (CDCl₃, 300 MHz): δ 8.10 (s, 1H), 7.99~7.94 (m, 2H), 7.85 (s, 1H), 7.64 (s, 1H), 7.50~7.47 (m, 2H), 7.30~7.27 (m, 1H), 7.24~7.18 (m, 3H), 7.08~7.14 (m, 1H), 6.95~6.89 (m, 1H), 5.86 (s, 1H), 4.08 (s, 3H), 3.18 (s, 3H), 3.00 (d, J = 4.2 Hz, 3H), 2.17 (s, 3H). | 617 |
| 521 | 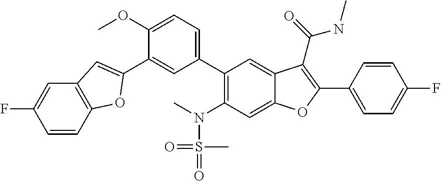 | ¹H-NMR (CDCl₃, 400 MHz): δ 8.11 (s, 1H), 7.95~8.10 (m, 1H), 7.86 (s, 1H), 7.64 (s, 1H), 7.48~7.50 (dd, J₁ = 1.0 Hz, J₂ = 8.0 Hz, 2H), 7.38~7.41 (m, 3H), 7.24~7.25 (d, J = 4.0 Hz, 1H), 6.96~7.28 (m, 3H), 5.87~5.88 (d, J = 4.0 Hz, 1H), 4.1 (s, 3H), 3.18 (s, 3H), 3.00~3.01 (d, J = 4.0 Hz, 3H), 2.73 (s, 3H). | 617 |
| 522 | 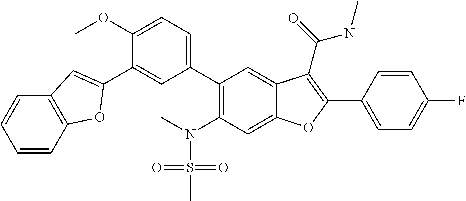 | ¹H-NMR (CDCl₃, 400 MHz) δ 8.10 (s, 1H), 7.92~7.95 (m, 2H), 7.82 (s, 1H), 7.58~7.32 (m, 2H), 7.43~7.46 (m, 2H), 7.40 (d, J = 1.6 Hz, 1H), 7.26~7.28 (m, 1H), 7.16~7.24 (m, 3H), 7.08 (d, J = 8.8 Hz, 1H), 5.91 (d, J = 4.8 Hz, 1H), 4.05 (s, 3H), 3.16 (s, 3H), 2.97 (d, J = 4.8 Hz, 3H), 2.88 (s, 3H). | 599 |
| 523 | 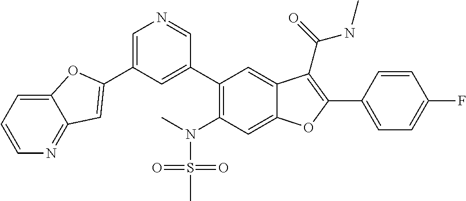 | ¹H-NMR (CDCl₃, 400 MHz) δ 9.17 (s, 1H), 8.75 (s, 1H), 8.61 (d, J = 4.8 Hz, 1H), 8.50 (s, 1H), 8.11 (d, J = 8.0 Hz, 1H), 7.93 (s, 1H), 7.83~7.86 (m, 2H), 7.72 (s, 1H), 7.59 (s, 1H), 7.48~7.49 (m, 1H), 7.14~7.16 (m, 2H), 5.89 (d, J = 4.8 Hz, 1H), 3.19 (s, 3H), 2.94 (d, J = 4.8 Hz, 3H), 2.81 (s, 3H). | 571 |
| 524 | 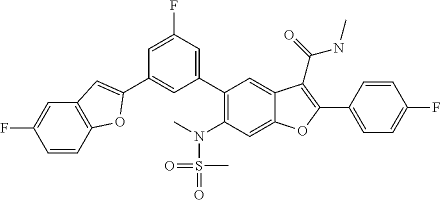 | ¹H-NMR (CDCl₃, 400 MHz): δ 7.93~7.96 (m, 2H), 7.90 (s, 1H), 7.78 (s, 1H), 7.64 (s, 1H), 7.59 (d, J = 8.0 Hz, 1H), 7.43~7.47 (m, 4H), 7.18~7.23 (m, 1H), 7.01~7.10 (m, 1H), 5.87 (s, 1H), 3.16 (s, 3H), 3.01 (d, J = 4.0 Hz, 3H), 2.81 (s, 3H). | 606 |
| 525 | 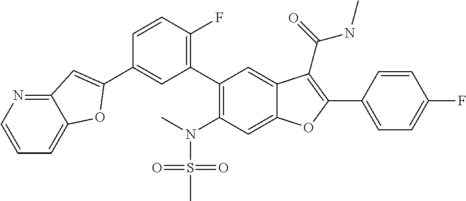 | ¹H-NMR (CDCl₃, 400 MHz) δ 8.51 (d, J = 4.8 Hz, 1H), 7.98~8.02 (m, 2H), 7.82~7.90 (m, 4H), 7.56 (s, 1H), 7.48 (s, 1H), 7.37~7.40 (m, 1H), 7.20~7.25 (m, 1H), 7.11~7.19 (m, 2H), 6.04 (d, J = 4.4 Hz, 1H), 3.22 (s, 3H), 2.91 (d, J = 4.8 Hz, 3H), 2.60 (s, 3H). | 588 |
| 526 | 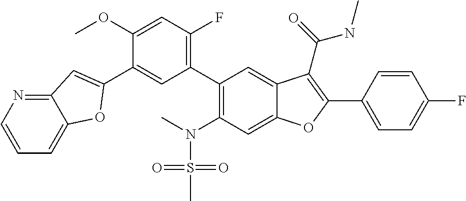 | ¹H-NMR (CDCl₃, 400 MHz) δ 8.48 (d, J = 4.4 Hz, 1H), 8.09 (d, J = 8.8 Hz, 1H), 7.91~7.95 (m, 2H), 7.85 (s, 1H), 7.64~7.69 (m, 2H), 7.44~7.50 (m, 2H), 7.14~7.21 (m, 2H), 6.84 (d, J = 11.6 Hz, 1H), 6.11 (br s, 1H), 4.05 (s, 3H), 3.26 (s, 3H), 2.97 (d, J = 4.8 Hz, 3H), 2.65 (s, 3H). | 618 |
| 527 | 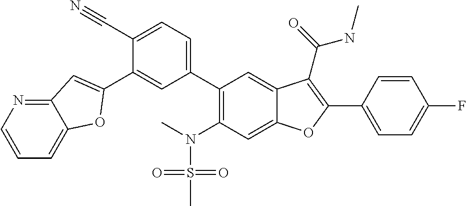 | ¹H-NMR (CDCl₃, 400 MHz) δ 8.60 (d, J = 4.4 Hz, 1H), 8.16 (s, 1H), 7.91~7.96 (m, 2H), 7.84~7.88 (m, 4H), 7.61~7.63 (m, 1H), 7.56 (d, J = 6.0 Hz, 1H), 7.35~7.45 (m, 1H), 7.15~7.17 (m, 2H), 5.80 (d, J = 4.4 Hz, 1H), 3.12 (s, 3H), 2.92 (d, J = 5.2 Hz, 3H), 2.82 (s, 3H) | 595 |
| 528 | 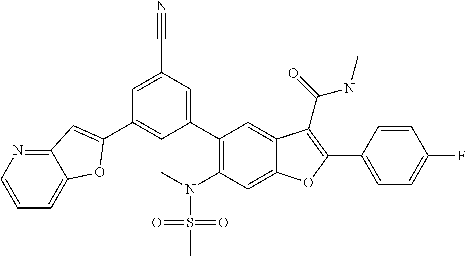 | ¹H-NMR (CDCl₃, 400 MHz) δ 8.47 (d, J = 4.4 Hz, 1H), 8.17 (s, 1H), 8.08 (s, 1H), 7.82~7.85 (m, 3H), 7.73 (d, J = 8.4 Hz, 1H), 7.65 (s, 1H), 7.54 (s, 1H), 7.29 (s, 1H), 7.17~7.20 (m, 1H), 7.13 (t, J = 8.4 Hz, 2H), 6.01 (d, J = 4.8 Hz, 1H), 3.11 (s, 3H), 2.92 (d, J = 4.4 Hz, 3H), 2.80 (s, 3H). | 595 |
| 529 | 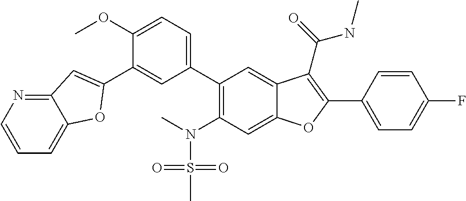 | ¹H-NMR (CDCl₃, 400 MHz) δ 8.50 (d, J = 3.6 Hz, 1H), 8.12 (d, J = 2.0 Hz, 1H), 7.91~7.95 (m, 2H), 7.84 (s, 1H), 7.72 (d, J = 8.4 Hz, 1H), 7.60 (d, J = 7.6 Hz, 2H), 7.49~7.53 (m, 1H), 7.16~7.21 (m, 3H), 7.10 (d, J = 8.4 Hz, 1H), 6.09 (br s, 1H), 3.16 (s, 3H), 2.97 (d, J = 4.8 Hz, 3H), 2.73 (s, 3H). | 600 |
| 530 | 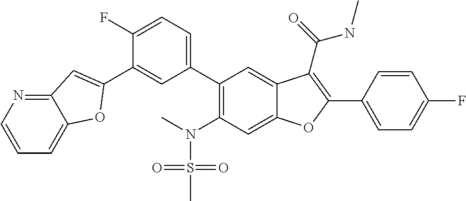 | ¹H-NMR (CDCl₃, 400 MHz) δ 8.56 (br s, 1H), 8.12 (br s, 1H), 7.89~7.95 (m, 3H), 7.81 (br s, 1H), 7.62 (s, 1H), 7.52 (m, 2H), 7.19~7.32 (m, 4H), 5.96 (br s, 1H), 3.17 (s, 3H), 2.99 (br s, 3H), 2.79 (s, 3H). | 588 |
| 531 | 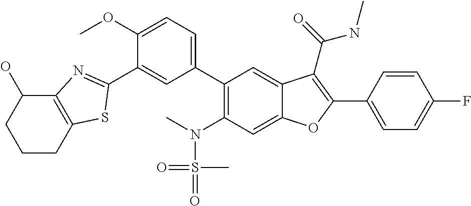 | ¹H-NMR (CDCl₃, 400 MHz) δ 8.55 (s, 1H), 8.04~8.07 (m, 2H), 7.86~7.89 (m, 2H), 7.55 (s, 1H), 7.17~7.23 (m, 4H), 5.01 (s, 1H), 4.15 (s, 3H), 3.11 (s, 3H), 3.00~3.03 (m, 3H), 2.90 (s, 3H), 2.78~2.82 (m, 2H), 1.94~2.14 (m, 4H). | 635 |
| 532 | 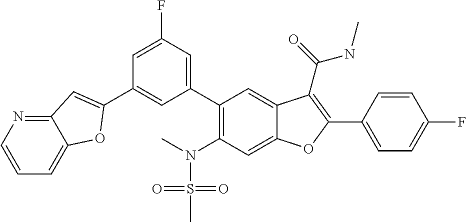 | ¹H-NMR (CDCl₃, 400 MHz) δ 8.54 (br s, 1H), 7.90~7.94 (m, 2H), 7.88 (s, 1H), 7.78~7.81 (m, 2H), 7.60~7.63 (m, 2H), 7.31 (s, 1H), 7.18~7.24 (m, 4H), 6.00 (br s, 1H), 3.17 (s, 3H), 2.99 (d, J = 4.4 Hz, 3H), 2.79 (s, 3H). | 588 |
| 533 | 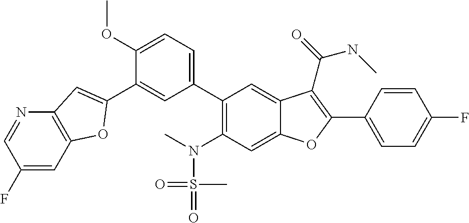 | ¹H-NMR (CDCl₃, 400 MHz) δ 8.48 (s, 1H), 8.11 (s, 1H), 7.96 (dd, J₁ = 5.2 Hz, J₂ = 8.8 Hz 2H), 7.89 (s, 1H), 7.63 (s, 1H), 7.61 (s, 1H), 7.56~7.52 (m, 2H), 7.20~7.25 (m, 2H), 7.11~7.14 (m, 1H), 4.09 (s, 3H), 3.17 (s, 3H), 3.00 (d, J = 4.4 Hz, 3H), 2.76 (d, 3H). | 618 |
| 534 | 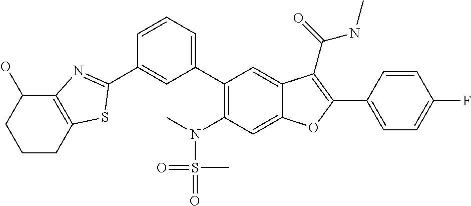 | ¹H-NMR (CDCl₃, 400 MHz): δ 8.09 (s, 1H), 7.89~7.93 (m, 3H), 7.80 (s, 1H), 7.50~7.62 (m, 3H), 7.14 (t, J = 8.8 Hz, 2H), 6.11 (br, 1H), 4.98 (s, 1H), 3.06 (s, 3H), 2.95 (d, J = 4.4 Hz, 3H), 2.82~2.88 (m, 3H), 2.73 (s, 3H), 1.85~2.07 (m, 3H). | 606 |
| 535 | 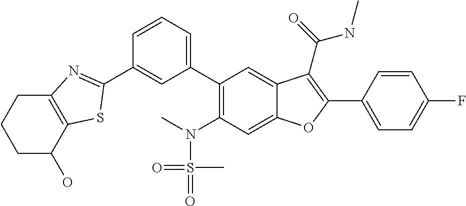 | ¹H-NMR (CDCl₃, 400 MHz): δ 7.91~8.06 (m, 4H), 7.84 (s, 1H), 7.55~7.62 (m, 3H), 7.21 (t, J = 8.4 Hz, 2H), 6.19 (br, 1H), 5.02 (s, 1H), 3.15 (s, 3H), 3.01 (d, J = 4.0 Hz, 3H), 2.71~2.92 (m, 2H), 2.41 (s, 3H), 1.92~2.17 (m, 5H). | 606 |
| 536 | 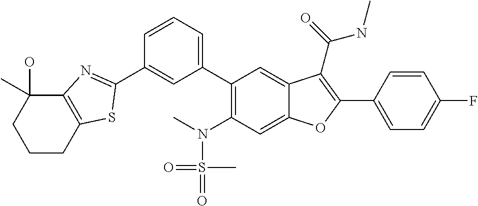 | ¹H-NMR (CDCl₃, 400 MHz): δ 7.86~8.06 (m, 5H), 7.83 (s, 1H), 7.50~7.65 (m, 2H), 7.21 (t, J = 8.8 Hz, 2H), 6.01 (br, 1H), 3.13 (s, 3H), 2.83∥3.00 (m, 4H), 2.41~2.68 (m, 8H), 1.73~2.17 (m, 3H). | 606 |
| 537 | 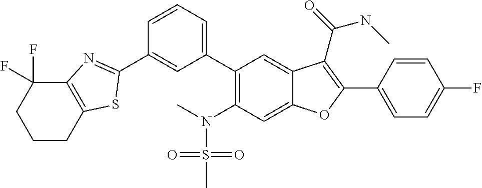 | ¹H-NMR (CDCl₃, 400 MHz): δ 8.07 (s, 1H), 8.00~7.95 (m, 3H), 7.84 (s, 1H), 7.64 (s, 1H), 7.60~7.50 (m, 2H), 7.21 (t, J = 7.6 Hz, 2H), 5.92 (br, 1H), 3.13 (s, 3H), 3.01 (d, J = 5.2 Hz, 3H), 2.98~2.90 (m, 2H), 2.72 (s, 3H), 2.42~2.33 (m, 2H), 2.17~2.13 (m, 2H). | 626 |
| 538 | 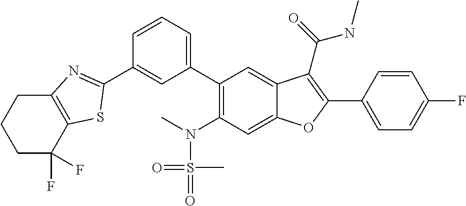 | ¹H-NMR (CDCl₃, 400 MHz) δ 8.03 (s, 1H), 7.93~7.97 (m, 3H), 7.84 (s, 1H), 7.64 (s, 1H), 7.54~7.60 (m, 2H), 7.21 (t, J = 8.8 Hz, 2H), 5.93 (br, 1H), 3.17 (s, 3H), 3.00 (d, J = 4.8 Hz, 3H), 2.92~2.95 (m, 2H), 2.67 (s, 3H), 2.31~2.41 (m, 2H), 2.09~2.16 (m, 2H). | 626 |
| 539 | 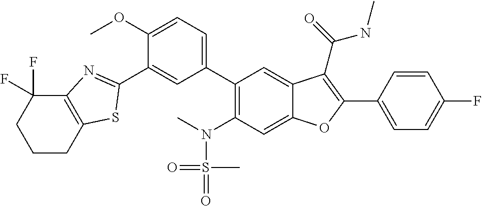 | ¹H-NMR (CDCl₃, 400 MHz) δ 8.50 (s, 1H), 7.96~8.00 (m, 2H), 7.80 (s, 1H), 7.57~7.63 (m, 2H), 7.11~7.22 (m, 3H), 6.05 (br, 1H), 4.08 (s, 1H), 3.11 (s, 3H), 3.01 (d, J = 4.8 Hz, 3H), 2.92~2.95 (m, 2H), 2.85 (s, 3H), 2.30~2.37 (m, 2H), 2.11~2.15 (m, 2H). | 656 |
| 540 | 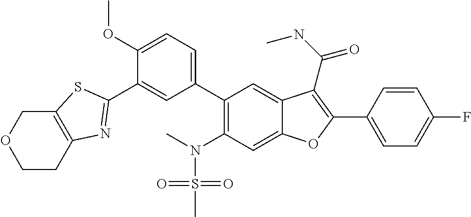 | ¹H-NMR (CDCl₃, 400 MHz) δ 8.45 (s, 1H), 8.00~8.04 (m, 2H), 7.83 (s, 1H) 7.69 (s, 1H), 7.60 (s, 1H), 7.14~7.22 (m, 3H), 6.45 (s, 1H), 4.90 (s, 2H), 4.10 (s, 3H), 4.06~4.08 (m, 2H), 3.11 (s, 3H), 3.01~3.02 (m, 5H), 2.84 (s, 3H). | 621 |
| 541 | 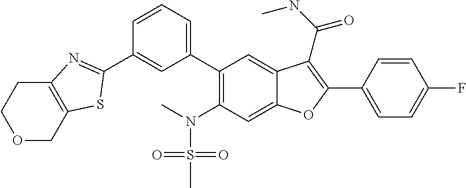 | ¹H-NMR (CDCl₃, 400 MHz) δ 7.95~ 8.01 (m, 3H), 7.93 (s, 1H), 7.85 (s, 1H), 7.63 (s, 1H), 7.52~7.56 (m, 2H), 7.20~7.24 (m, 2H), 5.97 (s, 1H), 4.88 (s, 2H), 4.05 (t, J = 5 Hz, 2H), 3.15 (s, 3H), 3.00 (d, J = 5 Hz, 5H), 2.71 (s, 3H). | 591 |
| 542 | 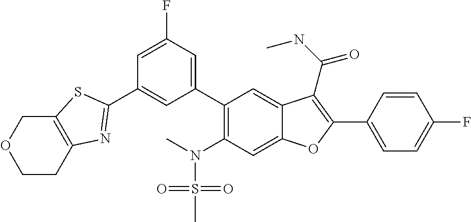 | ¹H-NMR (CDCl₃, 400 MHz) δ 7.93~7.96 (m, 2H), 7.86 (s, 1H), 7.79 (s, 1H), 7.68 (d, J = 9.2 Hz, 1H), 7.63 (s, 1H), 7.20~7.25 (m, 3H), 5.89 (d, J = 4.0 Hz, 1H), 4.88 (s, 2H), 4.06~4.09 (m, 2H), 3.16 (s, 3H), 3.01 (s, 2H), 3.00 (s, 3H), 2.79 (s, 3H). | 610 |
| 543 | 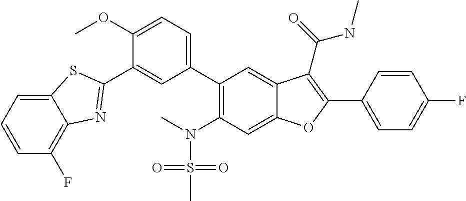 | ¹H-NMR (CDCl₃, 400 MHz) δ 8.62 (d, J = 2.0 Hz, 1H), 7.91~7.94 (m, 2H), 7.81 (s, 1H), 7.69 (d, J = 8.0 Hz, 1H), 7.61~7.65 (m, 2H), 7.29∥7.34 (m, 1H), 7.24 (s, 1H), 7.18 (d, J = 2.8 Hz, 1H), 7.14~7.16 (m, 2H), 6.16 (d, J = 4.4 Hz, 1H), 4.12 (s, 3H), 3.12 (s, 3H), 2.98 (d, J = 4.8 Hz, 3H), 2.86 (s, 3H). | 634 |
| 544 | 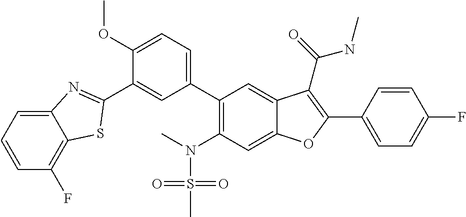 | ¹H-NMR (CDCl₃, 400 MHz) δ 8.51 (d, J = 2.4 Hz, 1H), 7.81~7.85 (m, 2H), 7.71~7.73 (m, 2H), 7.51~7.57 (m, 2H), 7.32~7.37 (m, 1H), 7.06~7.10 (m, 3H), 6.98~7.03 (m, 1H), 6.10~6.12 (d, J = 4.8 Hz, 1H), 4.06 (s, 3H), 3.04 (s, 3H), 2.88~2.89 (d, J = 4.8 Hz, 3H), 2.73 (s, 3H). | 634 |
| 545 | 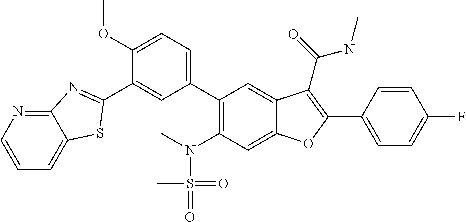 | ¹H-NMR (CDCl₃, 400 MHz) δ 8.85 (d, J = 3.6 Hz, 1H), 8.71 (s, 1H), 8.66 (t, J = 2.8 Hz, 1H), 7.87~7.91 (m, 2H), 7.82 (s, 2H), 7.60 (t, J = 2.8 Hz, 1H), 7.56 (s, 1H), 7.14 (t, J = 8.8 Hz, 1H), 6.40 (d, J = 5.2 Hz, 2H), 4.15 (s, 3H), 3.19 (s, 3H), 2.97 (s, 3H), 2.82 (s, 3H). | 617 |
| 546 | 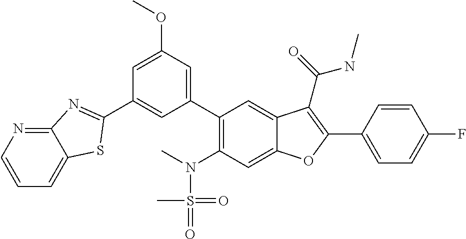 | ¹H-NMR (CDCl₃, 400 MHz) δ 8.73 (d, J = 4.0 Hz, 1H), 8.25~8.28 (m, 1H), 7.93~7.97 (m, 2H), 7.85 (s, 1H), 7.82 (d, J = 2.0 Hz, 2H), 7.61 (s, 1H), 7.31~7.35 (m, 1H), 7.17~7.23 (m, 3H), 5.94 (d, J = 4.4 Hz, 1H), 3.95 (s, 3H), 3.13 (s, 3H), 2.99 (d, J = 4.8 Hz, 3H), 2.81 (s, 3H). | 617 |
| 547 | 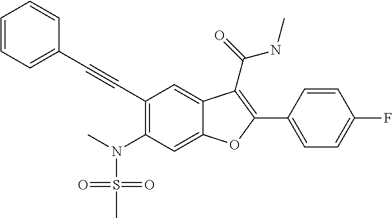 | ¹H-NMR (CDCl₃, 400 MHz) δ 8.07 (s, 1H), 7.90~7.93 (m, 2H), 7.65 (s, 1H), 7.51~7.52 (m, 2H), 7.39~7.40 (m, 3H), 7.17~7.29 (m, 2H), 5.87 (br s, 1H), 3.51 (s, 3H), 3.07 (s, 3H), 3.01 (d, J = 4.0 Hz, 3H). | 477 |
| 548 | 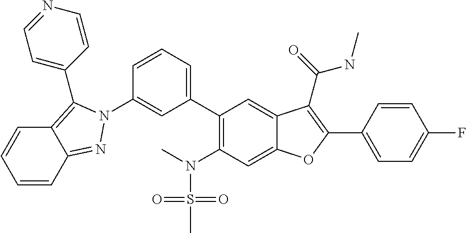 | ¹H-NMR (CDCl₃, 400 MHz) 8.71 (s, 2H), 7.85~7.87 (m, 3H), 7.74~7.79 (m, 4H), 7.71 (s, 1H), 7.42~7.49 (m, 4H), 7.28~7.34 (m, 2H), 7.12~7.17 (m, 2H), 5.79 (d, J = 4.4 Hz, 1H), 3.14 (s, 3H), 2.95 (d, J = 4.8 Hz, 3H), 2.85 (s, 3H). | 646 |
| 549 | 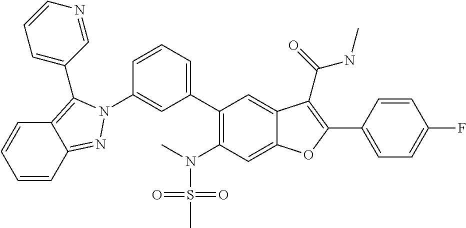 | ¹H-NMR (CDCl₃, 400 MHz) 8.92 (s, 1H), 8.17 (d, J = 4.8 Hz, 1H), 7.91~7.93 (m, 1H), 7.85~7.89 (m, 2H), 7.79~7.81 (m, 1H), 7.69~7.72 (m, 1H), 7.62~7.67 (m, 2H), 7.54 (s, 1H), 7.37~7.46 (m, 3H), 7.22~7.28 (m, 3H), 7.14 (t, J = 8.8 Hz, 2H), 5.83 (d, J = 3.6 Hz, 1H), 3.14 (s, 3H), 2.95 (d, J = 4.2 Hz, 3H), 2.76 (s, 3H). | 646 |
| 550 | 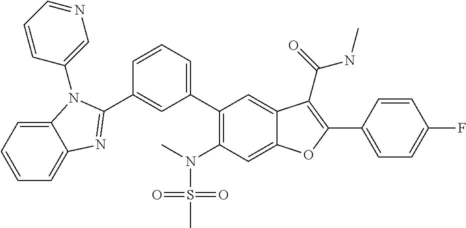 | ¹H-NMR (CDCl₃, 400 MHz) δ 8.80 (s, 2H), 7.99 (s, 1H), 7.91~7.94 (m, 2H), 7.80 (s, 1H), 7.73 (d, J = 7.6 Hz, 1H), 7.65 (s, 1H), 7.51~7.60 (m, 3H), 7.45 (t, J = 8.0 Hz, 2H), 7.38 (t, J = 8.0 Hz, 1H), 7.30 (d, J = 8.0 Hz, 2H), 7.13 (t, J = 8.4 Hz, 2H), 6.42 (s, 1H), 3.05 (s, 3H), 2.97 (d, J = 4.4 Hz, 3H), 2.74 (s, 3H). | 646 |
| 551 | 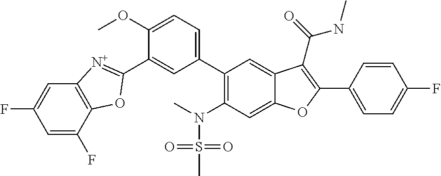 | NA | |
| 552 | 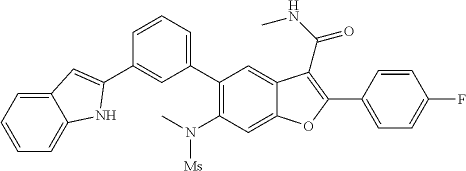 | ¹H-NMR (CDCl₃, 400 MHz) δ 9.30 (s, 1H), 7.94 (d, J = 8.8 Hz, 3H), 7.83 (s, 1H), 7.74 (d, J = 8.0 Hz, 1H), 7.65 (t, J = 7.2 Hz, 1H), 7.52~7.47 (m, 2H), 7.43 (d, J = 8.0 Hz, 1H), 7.35 (d, J = 6.8 Hz, 1H), 7.22~7.17 (m, 3H), 7.14~7.10 (m, 1H), 6.85 (s, 1H), 6.09 (d, J = 4.4 Hz, 1H), 2.99 (s, 3H), 2.97 (d, J = 4.0 Hz, 3H), 2.92 (s, 3H). | 568 |
| 553 | 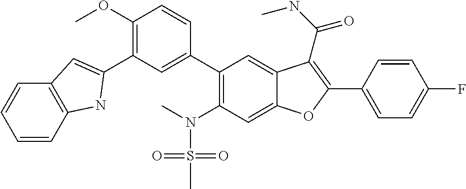 | ¹H-NMR (CDCl₃, 400 MHz) δ 9.74 (s, 1H), 7.98~8.01 (m, 2H), 7.87 (s, 1H), 7.64 (d, J = 10.8 Hz, 2H), 7.41~7.47 (m, 3H), 7.19~7.26 (m, 3H), 7.11~7.16 (m, 2H), 6.98 (s, 1H), 5.88 (d, J = 4.8 Hz, 1H), 4.11 (s, 3H), 3.15 (s, 3H), 3.02 (d, J = 4.8 Hz, 3H), 2.82 (s, 3H). | 598 |
| 554 | 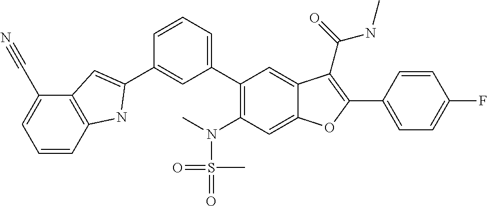 | ¹H-NMR (CDCl₃, 400 MHz) δ 9.71 (s, 1H), 9.73 (s, 1H), 7.83 (d, J = 6.0 Hz, 3H), 7.70 (d, J = 8.0 Hz, 1H), 7.55 (d, J = 8.0 Hz, 1H), 7.43~7.49 (m, 2H), 7.37 (t, J = 6.8 Hz, 2H), 7.13 (t, J = 8.2 Hz, 3H), 6.94 (s, 1H), 5.88 (s, 1H), 3.03 (s, 3H), 2.91 (d, J = 4.6 Hz, 3H), 2.85 (s, 3H). | 593 |
| 555 | 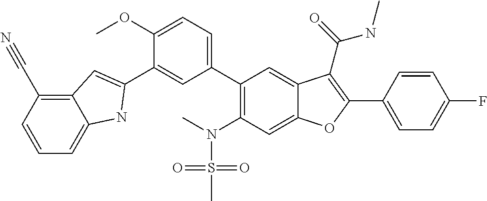 | ¹H-NMR (CDCl₃, 400 MHz) δ 9.97 (s, 1H), 7.86~7.91 (m, 3H), 7.79 (s, 1H), 7.56 (d, J = 8.4 Hz, 1H), 7.49 (s, 1H), 7.37~7.41 (m, 2H), 7.05~7.16 (m, 5H), 5.83 (d, J = 4.4 Hz, 1H), 4.02 (s, 3H), 3.02 (s, 3H), 2.93 (d, J = 4.8 Hz, 3H), 2.85 (s, 3H). | 623 |
| 556 | 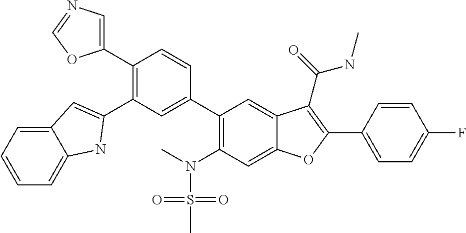 | ¹H-NMR (CDCl₃, 400 MHz) δ 8.83 (s, 1H), 7.78~7.79 (m, 6H), 7.52~7.61 (m, 3H), 7.40 (d, J = 8.4 Hz, 1H), 7.09~7.22 (m, 4H), 6.84 (d, J = 2.4 Hz, 1H), 6.48 (d, J = 1.4 Hz, 1H), 5.88 (s, 1H), 3.06 (d, J = 2.4 Hz, 3H), 2.99 (d, J = 2.8 Hz, 3H), 2.98 (d, J = 4.8 Hz, 3H). | 635 |
| 557 | 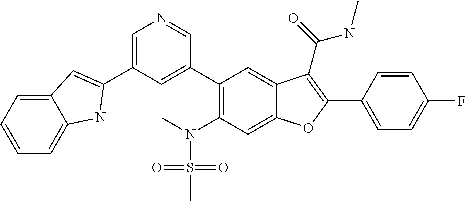 | ¹H-NMR (CDCl₃, 400 MHz) δ 9.43 (s, 1H), 8.94 (d, J = 1.6 Hz, 1H), 8.51 (d, J = 1.6 Hz, 1H), 8.22 (s, 1H), 7.86~7.91 (m, 3H), 7.62 (d, J = 7.6 Hz, 1H), 7.52 (s, 1H), 7.43 (d, J = 8.0 Hz, 1H), 7.09~7.22 (m, 4H), 6.88 (s, 1H), 6.08 (d, J = 4.8 Hz, 3H), 2.98 (s, 3H), 2.93 (d, J = 4.8 Hz, 3H). | 569 |
| 558 | 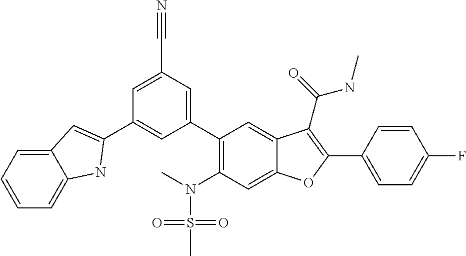 | ¹H-NMR (CDCl₃, 400 MHz) δ 9.28 (s, 1H), 8.04 (s, 1H), 7.78~7.83 (m, 4H), 7.55 (d, J = 7.6 Hz, 1H), 7.45 (d, J = 11.2 Hz, 2H), 7.32 (d, J = 8.0 Hz, 1H), 7.02~7.12 (m, 4H), 6.76 (s, 1H), 5.94 (s, 1H), 3.00 (s, 3H), 2.92 (s, 3H), 2.89 (s, 3H). | 593 |
| 559 | 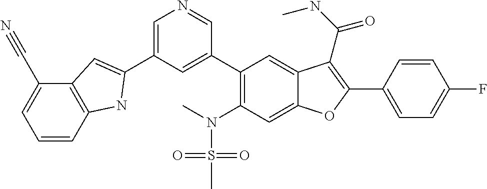 | ¹H-NMR (CDCl₃, 400 MHz) δ 10.31 (s, 1H), 8.81 (s, 1H), 8.50 (s, 1H), 8.15 (s, 1H), 7.81~7.85 (m, 2H), 7.77 (s, 1H), 7.59 (d, J = 8.4 Hz, 1H), 7.51 (s, 1H), 7.41 (d, J = 7.2 Hz, 1H), 7.12~7.20 (m, 3H), 6.85 (s, 1H), 6.47 (d, J = 4.4 Hz, 1H), 3.06 (s, 3H), 3.92 (s, 3H), 2.93 (d, J = 4.4 Hz, 3H). | 594 |
| 560 | 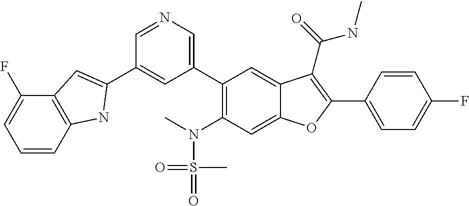 | ¹H-NMR (MeOD, 400 MHz) δ 8.97 (d, J = 0.12 Hz, 1H), 8.56 (s, 1H), 8.35 (s, 1H), 7.95~7.99 (m, 2H), 7.93 (s, 1H), 7.79 (s, 1H), 7.23~7.28 (m, 3H), 7.05~7.11 (m, 2H), 6.68~6.73 (m, 1H), 3.23 (s, 3H), 2.97 (s, 3H), 2.93 (s, 3H). | 587 |
| 561 | 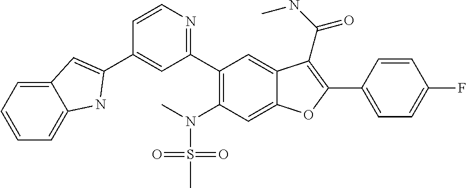 | ¹H-NMR (CDCl₃, 400 MHz) δ 9.48 (s, 1H), 8.64 (d, J = 5.2 Hz, 1H), 8.09 (d, J = 2.0 Hz, 1H), 8.05 (s, 1H), 7.94~7.97 (m, 2H), 7.64 (d, J = 8.0 Hz, 1H), 7.55 (d, J = 5.2 Hz, 1H), 7.53 (s, 1H), 7.43 (d, J = 8.0 Hz, 1H), 7.11~7.25 (m, 4H), 7.01 (s, 1H), 6.16 (s, 1H), 3.09 (d, J = 0.8 Hz, 3H), 3.03 (s, 3H), 2.98 (d, J = 4.8 Hz, 3H). | 569 |
| 562 | 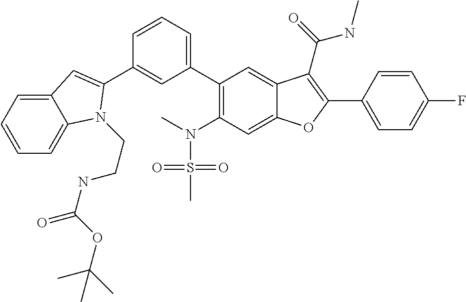 | ¹H-NMR (CDCl₃, 400 MHz) δ 7.96 (s, 1H), 7.87 (s, 1H), 7.40~7.56 (m, 7H), 7.05~7.18 (m, 4H), 6.51 (s, 1H), 5.98 (s, 1H), 4.94 (s, 1H), 4.15~4.25 (m, 2H), 3.32~3.33 (d, J = 5.6 Hz, 2H), 3.15 (s, 3H), 2.90 (s, 3H), 2.64 (s, 3H), 1.28 (s, 9H). | 711 |
| 563 | 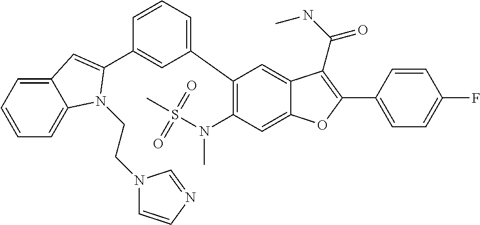 | ¹H-NMR (CDCl₃, 400 MHz) δ 8.18~8.11 (m, 3H), 7.71 (d, J = 7.6 Hz, 1H), 7.64 (s, 1H), 7.58 (s, 1H), 7.46~7.40 (m, 2H), 7.35~7.28 (m, 3H), 7.24~7.16 (m, 3H), 6.91 (s, 1H), 6.69 (s, 1H), 6.60 (s, 1H), 6.40 (s, 1H), 6.06 (s, 1H), 4.44 (d, J = 61.2 Hz, 4H), 3.29 (s, 3H), 3.04 (s, 3H), 2.61 (s, 3H). | 662 |
| 564 | 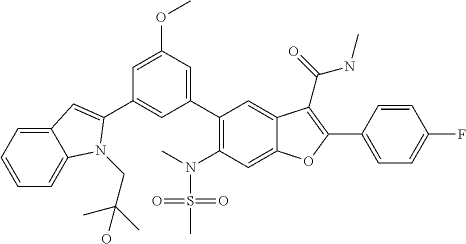 | ¹H-NMR (CDCl₃, 400 MHz) δ 7.96~7.98 (t, 2H), 7.83 (s, 1H), 7.53~7.64 (m, 3H), 7.02~7.24 (m, 7H), 6.61 (s, 1H), 5.82~5.83 (m, 1H), 4.42 (s, 2H), 3.90 (s, 3H), 3.25 (s, 3H), 2.99~3.00 (m, 3H), 2.85 (s, 3H), 1.73 (s, 1H), 1.00 (s, 6H). | 670 |
| 565 | 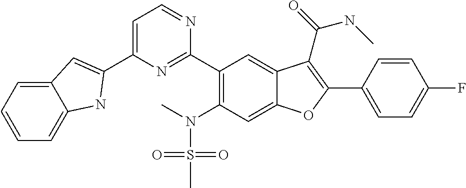 | ¹H-NMR (CDCl₃, 400 MHz) δ 10.59 (s, 1H), 8.76 (d, J = 5.6 Hz, 1H), 8.57 (s, 1H), 7.94~7.98 (m, 2H), 7.70 (d, J = 8.0 Hz, 1H), 7.62 (d, J = 5.2 Hz, 2H), 7.49 (d, J = 8.0 Hz, 1H), 7.25~7.31 (m, 2H), 7.14~7.19 (m, 3H), 6.19 (d, J = 4.8 Hz, 1H), 3.27 (s, 3H), 3.11 (s, 3H), 2.97 (d, J = 4.8 Hz, 3H). | 570 |
| 566 | 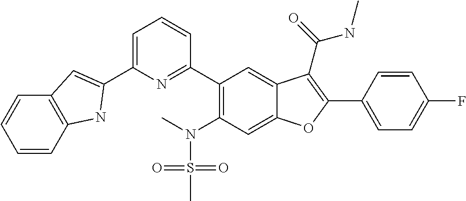 | ¹H-NMR (CDCl₃, 400 MHz) δ 9.76 (s, 1H), 7.94 (s, 1H), 7.87~7.89 (m, 2H), 7.71 (d, J = 0.28 Hz, 2H), 7.58 (d, J = 7.6 Hz, 1H), 7.54 (s, 1H), 7.33~7.37 (m, 2H), 7.13 (t, J = 8.4 Hz, 3H), 7.03 (t, J = 7.4 Hz, 1H), 5.93 (s, 1H), 3.59~3.64 (m, 1H), 3.08 (s, 3H), 2.88 (d, J = 4.8 Hz, 3H), 2.75 (s, 3H). | 569 |
| 567 | 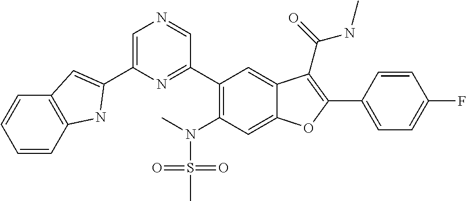 | ¹H-NMR (CDCl₃, 400 MHz) δ 9.82 (s, 1H), 9.00 (s, 1H), 8.60 (s, 1H), 8.09 (s, 1H), 7.87~7.91 (m, 2H), 7.61 (t, J = 9.6 Hz, 2H), 7.38 (d, J = 8.0 Hz, 1H), 7.05~7.18 (m, 5H), 5.83 (d, J = 4.8 Hz, 1H), 3.13 (s, 3H), 2.92 (d, J = 5.2 Hz, 3H), 2.88 (s, 3H). | 570 |
| 568 | 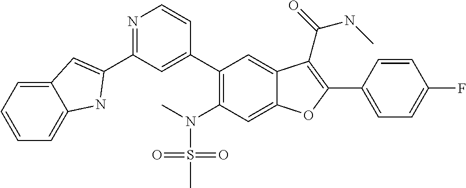 | ¹H-NMR (MeOD, 400 MHz) δ 8.70 (d, J = 6.0 Hz, 1H), 8.41 (s, 1H), 8.12 (s, 1H), 8.00~8.03 (m, 3H), 7.88 (d, J = 5.6 Hz, 1H), 7.73 (d, J = 8.0 Hz, 1H), 7.50~7.55 (m, 2H), 7.32~ 7.36 (m, 3H), 7.18 (d, J = 7.6 Hz, 1H), 3.46 (s, 3H), 3.03 (s, 3H), 2.98 (s, 3H). | 569 |
| 569 | 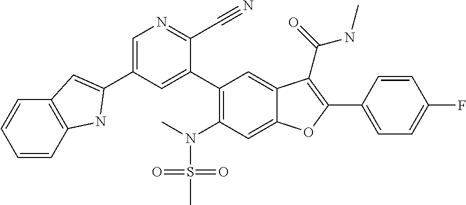 | ¹H-NMR (CDCl₃, 400 MHz) δ 9.00 (s, 1H), 8.92 (d, J = 2.0 Hz, 1H), 8.15 (d, J = 2.0 Hz, 1H), 7.86~7.89 (m, 3H), 7.56 (d, J = 8.0 Hz, 2H), 7.31 (d, J = 8.0 Hz, 1H), 7.05~7.18 (m, 4H), 6.87 (s, 1H), 5.87 (d, J = 4.4 Hz, 1H), 3.18 (s, 3H), 2.91 (d, J = 4.8 Hz, 3H), 2.90 (s, 3H). | 594 |
| 570 | 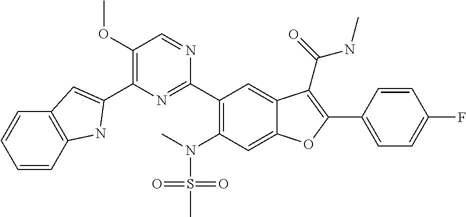 | ¹H-NMR (CDCl₃, 400 MHz) δ 10.24 (s, 1H), 8.46 (s, 1H), 8.38 (s, 1H), 7.93~7.97 (m, 2H), 7.60~7.65 (m, 1H), 7.52 (d, J = 1.2 Hz, 1H), 7.43 (t, J = 8.8 Hz, 1H), 7.23 (t, J = 8.0 Hz, 1H), 7.04~7.16 (m, 4H), 5.95 (d, J = 4.0 Hz, 1H), 4.12 (s, 3H), 3.19 (s, 3H), 2.95 (d, J = 4.4 Hz, 6H). | 600 |
| 571 | 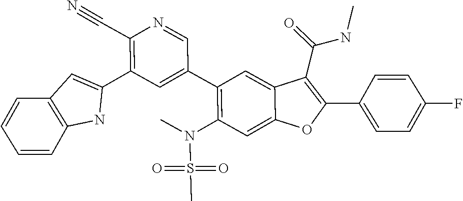 | ¹H-NMR (CDCl₃, 400 MHz) δ 9.43 (s, 1H), 8.49 (d, J = 1.6 Hz, 1H), 8.26 (d, J = 2.0 Hz, 1H), 7.90 (s, 1H), 7.77~7.81 (m, 2H), 7.59 (d, J = 7.6 Hz, 1H), 7.45 (s, 1H), 7.34 (d, J = 8.8 Hz, 1H), 7.24 (d, J = 1.2 Hz, 1H), 7.04~7.16 (m, 4H), 5.92 (d, J = 4.4 Hz, 1H), 3.06 (s, 3H), 2.92 (d, J = 4.4 Hz, 6H). | 594 |
| 572 | 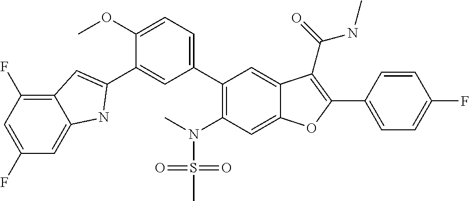 | ¹H-NMR (CDCl₃, 400 MHz) δ 9.77 (s, 1H), 7.85 (s, 1H), 7.76 (s, 1H), 7.50 (s, 1H), 7.33 (d, J = 7.6 Hz, 1H), 7.14 (t, J = 8.0 Hz, 2H), 7.04 (d, J = 8.2 Hz, 1H), 6.92 (s, 1H), 6.86 (d, J = 8.6 Hz, 1H), 6.53 (t, J = 8.8 Hz, 1H), 5.91 (d, J = 4.0 Hz, 1H), 4.00 (s, 3H), 2.94 (d, J = 4.8 Hz, 3H), 2.93 (s, 3H), 2.84 (s, 3H). | 634 |
| 573 | 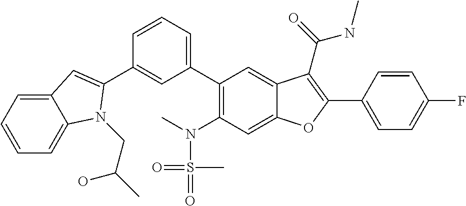 | ¹H-NMR (MeOD, 400 MHz) δ 7.86~7.90 (m, 2H), 7.73 (s, 1H), 7.51~7.59 (m, 2H), 7.37~7.49 (m, 5H), 7.14~7.19 (m, 2H), 7.07 (t, J = 7.6 Hz, 1H), 6.96 (t, J = 7.4 Hz, 1H), 6.47 (s, 1H), 3.88~4.31 (m, 3H), 3.12 (s, 3H), 2.83 (s, 3H), 2.76 (s, 3H), 0.80 (t, J = 4.8 Hz, 3H). | 626 |
| 574 | 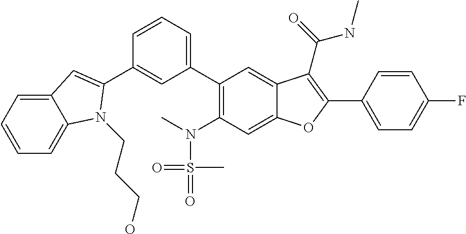 | ¹H-NMR (CDCl₃, 400 MHz) δ 7.82~7.96 (m, 3H), 7.34~7.64 (m, 6H), 7.13~7.24 (m, 5H), 6.58 (d, J = 8.8 Hz, 1H), 5.98 (d, J = 4.4 Hz, 1H), 4.35~4.41 (m, 2H), 3.54 (t, J = 5.8 Hz, 2H), 3.20 (s, 3H), 2.95~2.97 (m, 3H), 2.73 (s, 3H), 1.95~2.06 (m, 2H). | 626 |
| 575 | 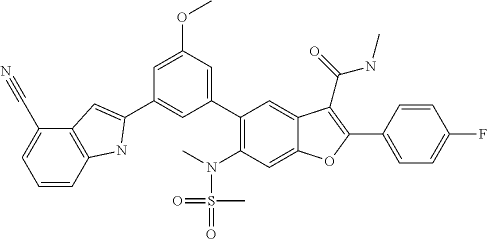 | ¹H-NMR (CDCl₃, 400 MHz) δ 9.86 (s, 1H), 7.82~7.86 (m, 2H), 7.78 (s, 1H), 7.58 (d, J = 8.4 Hz, 1H), 7.46 (s, 1H), 7.33~7.42 (m, 2H), 7.20 (s, 1H), 7.12~7.16 (m, 3H), 6.92 (s, 2H), 6.09 (d, J = 4.8 Hz, 1H), 3.86 (s, 3H), 3.04 (s, 3H), 2.92 (d, J = 4.8 Hz, 3H), 2, 90 (s, 3H). | 623 |
| 576 | 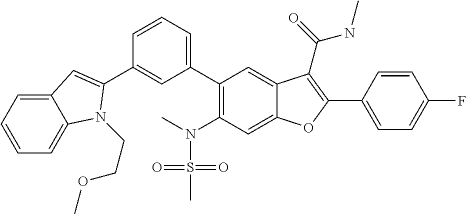 | ¹H-NMR (CDCl₃, 400 MHz) δ 7.92~7.98 (m, 2H), 7.85 (s, 1H), 7.50~7.65 (m, 5H), 7.43~7.49 (m, 2H), 7.13~7.25 (m, 4H), 6.58 (d, J = 0.4 Hz, 1H), 5.88 (d, J = 4.4 Hz, 1H), 4.39 (d, J = 6.0 Hz, 2H), 3.63 (d, J = 6.4 Hz, 2H), 3.22 (s, 3H), 3.18 (s, 3H), 2.99 (d, J = 4.8 Hz, 3H), 2.61 (s, 3H). | 626 |
| 577 | 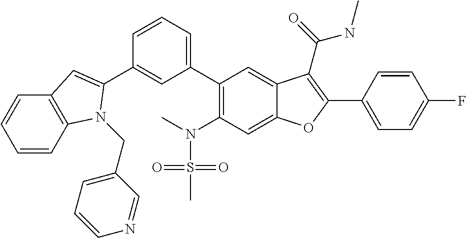 | ¹H-NMR (CDCl₃, 400 MHz) δ 8.32~8.38 (m, 2H), 7.95~7.98 (m, 2H), 7.74 (s, 1H), 7.66~7.68 (m, 1H), 7.56 (s, 1H), 7.39~7.47 (m, 4H), 7.10~7.22 (m, 6H), 6.69 (s, 1H), 6.31 (s, 1H), 5.44 (s, 2H), 5.28 (s, 1H), 3.10 (s, 3H), 2.99~3.01 (m, 3H), 2.55 (s, 3H). | 659 |
| 578 | 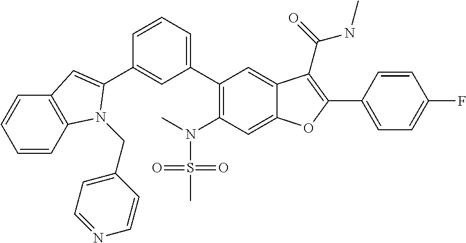 | ¹H-NMR (CDCl₃, 400 MHz) δ 8.43~8.44 (m, 2H), 7.92~7.96 (m, 2H), 7.70~7.76 (m, 2H), 7.58 (s, 1H), 7.39~7.47 (m, 4H), 7.17~7.23 (m, 6H), 6.90~6.91 (m, 2H), 6.74 (s, 1H), 5.43 (s, 2H), 3.12 (s, 3H), 2.99~3.00 (d, J = 4.8 Hz, 3H), 2.58 (s, 3H). | 659 |
| 579 | 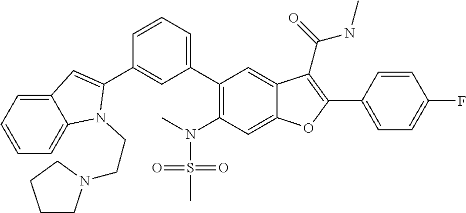 | ¹H-NMR (CDCl₃, 400 MHz) δ 7.86~7.89 (m, 2H), 7.79 (s, 1H), 7.52~7.53 (m, 3H), 7.47~7.48 (d, J = 4.0 Hz, 2H), 7.40~7.43 (m, 4H), 7.05~7.22 (m, 2H), 6.51 (s, 1H), 5.93~5.94 (d, J = 4.8 Hz, 1H), 4.37~4.41 (t, J = 3.2 Hz, 2H), 3.13 (s, 3H), 2.91~2.92 (d, J = 4.8 Hz, 3H), 2.74~2.77 (t, J = 7.2 Hz, 2H), 2.61 (s, 3H), 2.56~2.50 (m, 4H), 1.69 (s, 4H). | 665 |
| 580 | 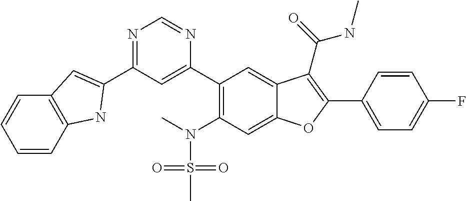 | ¹H-NMR (CDCl₃, 400 MHz) δ 9.65 (s, 1H), 9.25 (s, 1H), 8.21~8.22 (d, J = 1.2 Hz, 1H), 8.13 (s, 1H), 7.95~7.99 (m, 2H), 7.60~7.65 (m, 2H), 7.38~7.45 (m, 2H), 7.26~7.30 (m, 1H), 7.09~7.16 (m, 3H), 6.35 (s, 1H), 3.23 (s, 3H), 2.97~2.98 (d, J = 4.8 Hz, 3H), 2.95 (s, 3H). | 570 |
| 581 | 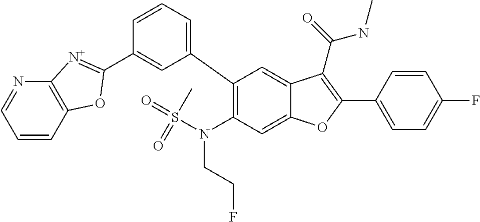 | NA | |
| 582 | 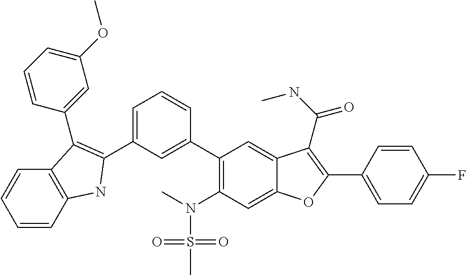 | ¹H-NMR (CDCl₃, 400 MHz) 9.40 (s, 1H), 7.92 (d, J = 4.2 Hz, 3H), 7.85 (s, 1H), 7.69 (d, J = 8.0 Hz, 1H), 7.47 (d, J = 8.0 Hz, 2H), 7.36~7.48 (m, 1H), 7.30~7.33 (m, 3H), 7.05~7.23 (m, 4H), 6.85~6.87 (m, 2H), 5.96 (d, J = 4.8 Hz, 1H), 3.79 (s, 3H), 3.06 (s, 3H), 2.98 (d, J = 4.8 Hz, 3H), 2.87 (s, 3H). | 674 |
| 583 | 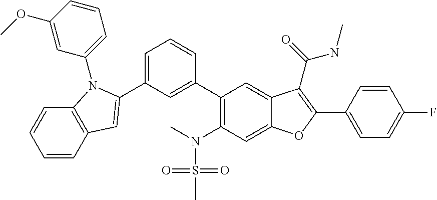 | ¹H-NMR (CDCl₃, 400 MHz) 7.92 (d, J = 2.0 Hz, 2H), 7.68~7.70 (m, 1H), 7.63 (s, 1H), 7.57 (s, 1H), 7.41 (s, 1H), 7.28~7.35 (m, 5H), 7.18~7.22 (m, 4H), 6.85~6.92 (m, 2H), 6.8 (s, 1H), 6.79 (d, J = 2.0 Hz, 1H), 5.85 (d, J = 4.0 Hz, 1H), 3.74 (s, 3H), 3.06 (s, 3H), 3.00 (d, J = 4.8 Hz, 3H), 2.55 (s, 3H). | 674 |
| 584 | 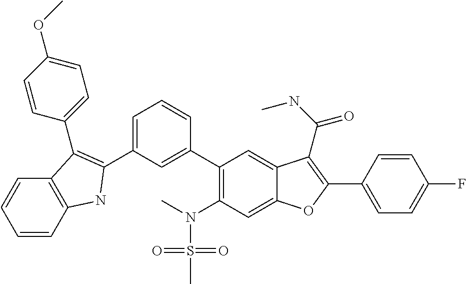 | ¹H-NMR (CDCl₃, 400 MHz) 9.33 (s, 1H), 7.93~7.97 (m, 3H), 7.87 (s, 1H), 7.64 (d, J = 8.0 Hz, 1H), 7.50 (s, 1H), 7.46 (d, J = 8.0 Hz, 1H), 7.41 (d, J = 8.0 Hz, 2H), 7.33~7.35 (m, 1H), 7.30 (d, J = 4.0 Hz, 2H), 7.17~7.24 (m, 3H), 7.12 (t, J = 8.0 Hz, 1H), 6.95 (d, J = 8.0 Hz, 2H), 5.93 (d, J = 4.0 Hz, 1H), 3.86 (s, 3H), 3.07 (s, 3H), 2.99 (d, J = 4.8 Hz, 3H), 2.88 (s, 3H). | 674 |
| 585 | 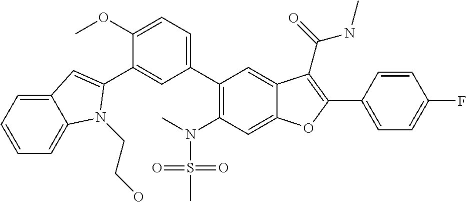 | ¹H-NMR (CDCl₃, 400 MHz) δ 7.96~7.93 (m, 2H), 7.86 (s, 1H), 7.64 (d, J = 7.6 Hz, 1H), 7.55 (s, 1H), 7.51~7.48 (m, 1H), 7.43~7.42 (m, 2H), 7.22 (t, J = 8.0 Hz, 3H), 7.18 (s, 1H), 7.14~7.08 (m, 1H), 6.52 (s, 1H), 5.86 (d, J = 4.4 Hz, 1H), 4.26 (t, J = 6.0 Hz, 2H), 3.85 (s, 3H), 3.79 (t, J = 6.0 Hz, 2H), 3.21 (s, 3H), 3.00 (d, J = 1.6 Hz, 3H), 2.79 (s, 3H). | 642 |
| 586 | 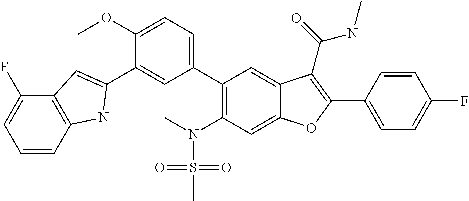 | ¹H-NMR (CDCl₃, 400 MHz) δ 9.79 (s, 1H), 7.91~7.94 (m, 3H), 7.82 (s, 1H), 7.57 (s, 1H), 7.38~7.41 (d, J = 8.4 Hz, 1H), 7.16~7.21 (m, 3H), 7.00~7.11 (m, 3H), 6.71~6.76 (t, 1H), 5.88 (s, 1H), 4.06 (s, 3H), 3.09 (s, 3H), 2.98 (s, 3H), 2.81 (s, 3H). | 616 |
| 587 | 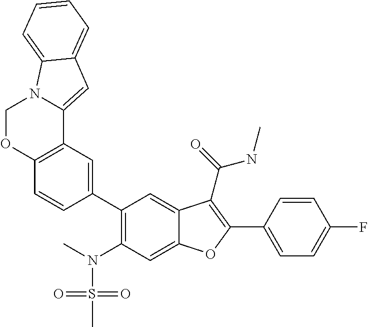 | ¹H-NMR (CDCl₃, 400 MHz) δ 9.48 (s, 1H), 8.10 (s, 1H), 7.95~7.93 (m, 2H), 7.91 (t, J = 2.0 Hz, 1H), 7.73 (d, J = 8.0 Hz, 1H), 7.60 (s, 1H), 7.50 (t, J = 7.6 Hz, 1H), 7.37~7.32 (m, 2H), 7.25~7.18 (m, 3H), 6.94~6.89 (m, 1H), 6.79 (s, 1H), 5.89 (d, J = 4.0 Hz, 1H), 3.67 (t, J = 10.8 Hz, 1H), 3.46 (t, J = 3.6 Hz, 2H), 3.26 (s, 3H), 2.99 (d, J = 4.8 Hz, 4H). | 596 |
| 588 | 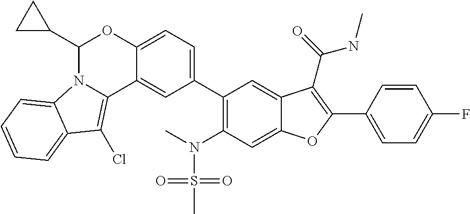 | ¹H-NMR (CDCl₃, 400 MHz) δ 8.39 (s, 1H), 7.95 (t, J = 8.0 Hz, 2H), 7.85 (s, 1H), 7.64 (d, J = 6.8 Hz, 2H), 7.40 (d, J = 4.4 Hz, 2H), 7.30 (d, J = 7.2 Hz, 1H), 7.23~7.20 (m, 4H), 5.94 (s, 1H), 5.77 (d, J = 7.2 Hz, 1H), 3, 29 (s, 3H), 3.00 (d, J = 4.8 Hz, 3H), 2.62 (s, 3H), 1.55~1.50 (m, 1H), 0.66~0.60 (m, 4H). | 670 |
| 589 | 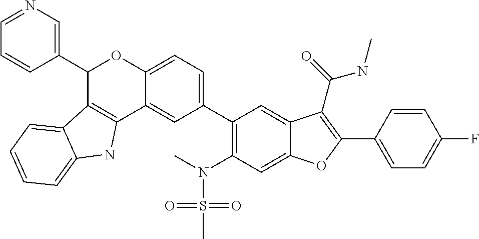 | ¹H-NMR (CDCl₃, 400 MHz) δ 8.49 (s, 1H), 8, 48 (s, 1H), 7.95~7.91 (m, 2H), 7.83 (d, J = 7.6 Hz, 1H), 7.75 (d, J = 6.8 Hz, 1H), 7.66 (s, 2H), 7.39 (t, J = 8.4 Hz, 2H), 7.26~7.21 (m, 3H), 7, 12~7.09 (m, 1H), 6.96~6.90 (m, 3H), 6.76 (d, J = 12.8 Hz, 1H), 3.20 (s, 3H), 2.91 (s, 3H), 2.77 (s, 3H). | 673 |
| 590 | 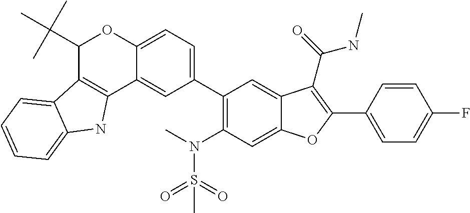 | ¹H-NMR (CDCl₃, 400 MHz) δ 8.95 (s, 1H), 7.79~7.83 (m, 2H), 7.63 (s, 1H), 7.41~7.50 (m, 4H), 7.07~7.20 (m, 5H), 6.87 (d, J = 8.0 Hz, 1H), 6.08 (d, J = 4.4 Hz, 1H), 2.95 (d, J = 4.8 Hz, 3H), 2.92 (s, 6H), 1.85~2.16 (m, 8H). | 652 |
| 591 | 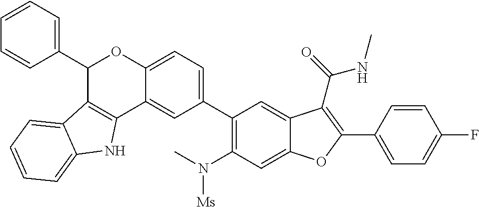 | ¹H-NMR (CDCl₃, 400 MHz) δ 8.96 (s, 1H), 7.88~7.92 (m, 2H), 7.75 (s, 1H), 7.65 (s, 1H), 7.32~7.49 (m, 7H), 7.14~7.18 (m, 4H), 6.96~6.99 (m, 2H), 6.86 (s, 1H), 6.55 (s, 1), 6.91 (d, J = 4.8 Hz, 1H), 3.02 (s, 3H), 2.98 (d, J = 4.8 Hz, 3H), 2.94 (s, 3H). | 672 |
| 592 | 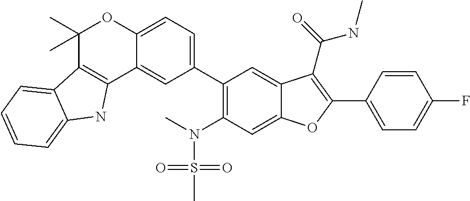 | ¹H-NMR (CDCl₃, 400 MHz) δ 8.89 (s, 1H), 7.86 (d, J = 5.2 Hz, 2H), 7.30 (d, J = 2.4 Hz, 1H), 7.60 (s, 1H), 7.55 (d, J = 8.0 Hz, 1H), 7.48 (s, 1H), 7.43 (d, J = 8.0 Hz, 1H), 7.22~7.11 (m, 5H), 6.95 (d, J = 4.4 Hz, 1H), 6.0 (s, 1H), 2.98~2.93 (t, J = 8.8 Hz, 9H), 1.78 (s, 6H). | 624 |
| 593 | 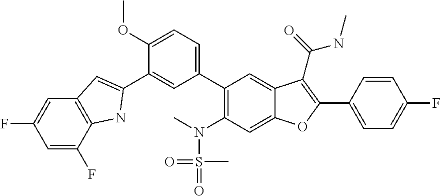 | ¹H-NMR (MeOD, 400 MHz) δ 7.95~7.98 (m, 2H), 7.91~7.92 (d, J = 2.0 Hz, 1H), 7.79 (s, 1H), 7.70 (s, 1H), 7.46~7.49 (m, 1H), 7.22~7.28 (q, 3H), 7.02~7.05 (m, 1H), 6.96~6.97 (d, J = 3.2 Hz, 1H), 6.66~6.72 (m, 1H), 4.03 (s, 3H), 3.12 (s, 3H), 2.93 (s, 6H). | 634 |
| 594 | 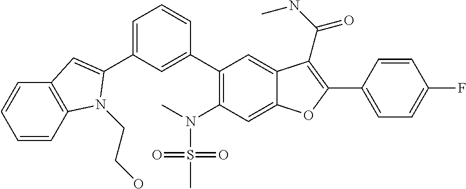 | ¹H-NMR (CDCl₃, 400 MHz) δ 7.89~7.93 (m, 2H), 7.82~7.84 (m, 1H), 7.57~7.65 (m, 2H), 7.53~7.55 (m, 3H), 7.42~7.44 (m, 2H), 7.13~7.24 (m, 4H), 6.59~6.60 (m, 1H), 6.07~6.08 (m, 1H), 4.36~4.39 (m, 2H), 3.84~3.87 (t, 1H), 3.74 (s, 1H), 3.20~3.25 (m, 3H), 2.97~2.98 (m, 3H), 2.77 (s, 3H). | 612 |
| 595 | 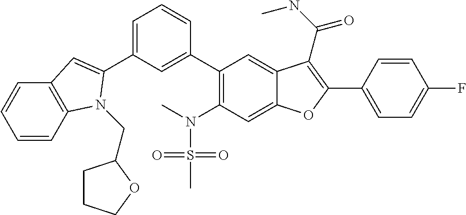 | ¹H-NMR (CDCl₃, 400 MHz) δ 7.93~7.97 (m, 2H), 7.83 (s, 1H), 7.60~7.64 (m, 2H), 7.51~7.56 (m, 3H), 7.46~7.48 (m, 1H), 7.39~7.41 (m, 1H), 7.12~7.23 (m, 4H), 6.58 (s, 1H), 5.91 (s, 1H), 4.26~4.28 (d, J = 8.0 Hz, 2H), 3.63~3.68 (m, 1H), 3.52~3.58 (m, 1H), 3.45~3.49 (m, 1H), 3.33~3.36 (m, 1H), 3.22 (s, 3H), 2.97~2.98 (m, 3H), 2.69 (s, 3H), 2.62~2.65 (m, 1H), 1.70~1.80 (m, 1H), 1.35~1.45 (m, 1H). | 652 |
| 596 | 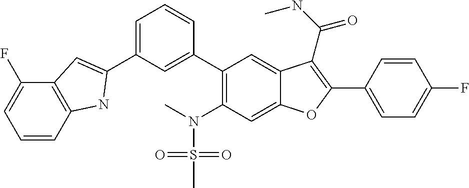 | ¹H-NMR (CDCl₃, 400 MHz) δ 9.28 (s, 1H), 7.89~7.95 (m, 4H), 7.73 (d, J = 7.6 Hz, 1H), 7.51 (m, 2H), 7.38 (d, J = 7.6 Hz, 1H), 7.17~7.21 (m, 3H), 7.03~7.09 (m, 1H), 6.88 (s, 1H), 6.72~6.77 (m, 1H), 5.83 (s, 1H), 3.04 (s, 3H), 2.98 (d, J = 8.8 Hz, 3H), 2.92 (s, 3H). | 586 |
| 597 | 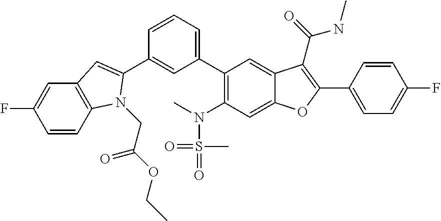 | ¹H-NMR (CDCl₃, 400 MHz) δ 7.95~7.97 (m, 2H), 7.83 (s, 1H), 7.51~7.60 (m, 5H), 7.14~7.31 (m, 4H), 6.96~7.01 (m, 1H), 6.61 (s, 1H), 5.92 (d, J = 4.0 Hz, 1H), 4.84 (s, 2H), 4.14~4.19 (s, 2H), 3.19 (s, 3H), 2.98 (d, J = 4.8 Hz, 3H), 2.65 (s, 3H), 1.18 (t, J = 7.0 Hz, 3H). | 672 |
| 598 | 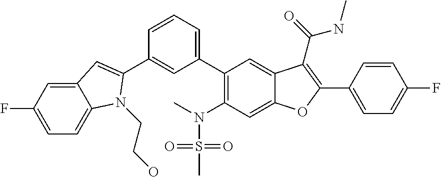 | ¹H-NMR (CDCl₃, 400 MHz) δ 7.92~7.96 (m, 2H), 7.88 (s, 1H), 7.45~7.62 (m, 5H), 7.28~7.36 (m, 4H), 6.95~7.23 (m, 1H), 6.55 (s, 1H), 5.86 (s, 1H), 4.34 (t, J = 6.4 Hz, 2H), 3.83 (t, J = 6.4 Hz, 2H), 3.26 (s, 3H), 2.98 (d, J = 4.8 Hz, 3H), 2.78 (s, 3H). | 630 |
| 599 | 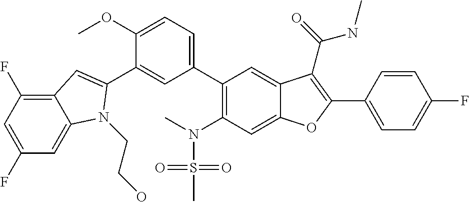 | ¹H-NMR (CDCl₃, 400 MHz) δ 7.92~7.95 (m, 2H), 7.86 (s, 1H), 7.54 (s, 1H), 7.49~7.51 (m, 1H), 7.43 (d, J = 2.4 Hz, 1H), 7.20 (t, J = 8.0 Hz, 2H), 7.08 (d, J = 8.0 Hz, 1H), 6.93 (s, 1H), 6.90 (d, J = 0.8 Hz, 1H), 6.58~6.64 (m, 1H), 6.55 (s, 1H), 5.87 (d, J = 4.0 Hz, 1H), 4.13 (t, J = 6.0 Hz, 2H), 3.86 (s, 3H), 2.84 (s, 2H), 3.21 (s, 3H), 2.99 (d, J = 4.8 Hz, 3H), 2.84 (s, 3H). | 678 |
| 600 | 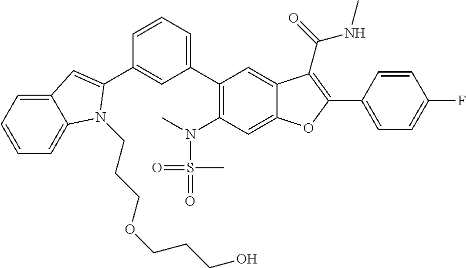 | ¹H-NMR (CDCl₃, 400 MHz) δ: 7.90~7.92 (m, 2H), 7.85 (d, J = 6.8 Hz, 1H), 7.53~7.64 (m, 5H), 7.46~7.48 (m, 1H), 7.40 (d, J = 8.0 Hz, 1H), 7.10~7.22 (m, 4H), 6.57 (s, 1H), 5.88~6.09 (m, 1H), 4.33~4.38 (m, 2.5H), 3.64 (t, J = 5.8 Hz, 1.5H), 3.30~3.35 (m, 2H), 3.20 (s, 5H), 2.96 (d, J = 4.8 Hz, 3H), 2.63 (s, 3H), 1.86~1.90 (m, 2.5H), 1.64~1.70 (m, 1.5H). | 684 |
| 601 | 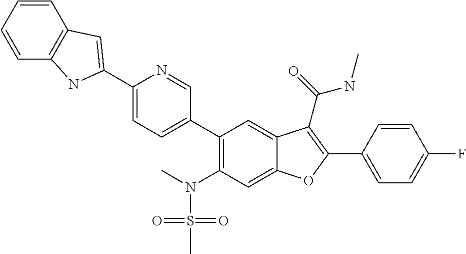 | ¹H-NMR (DMSO-d6, 400 MHz) δ 11.71 (s, 1H), 8.67 (d, J = 0.16 Hz, 1H), 8.50 (d, J = 4.8 Hz, 1H), 8.08 (s, 1H), 7.98~8.03 (m, 3H), 7.90~7.93 (m, 1H), 7.70 (s, 1H), 7.55 (d, J = 7.6 Hz, 1H), 7.37~7.45 (m, 3H), 7.18 (s, 1H), 7.08·7.12 (t, J = 7.4 Hz, 1H), 6.98 (t, J = 7.4 Hz, 1H), 3.16 (s, 3H), 3.01 (s, 3H), 2.80 (d, J = 4.0 Hz, 3H). | 569 |
| 602 | 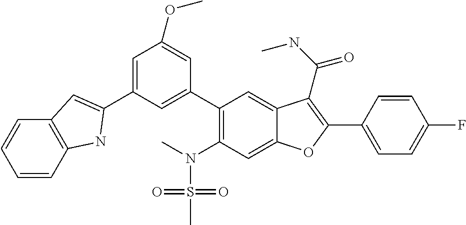 | ¹H-NMR (CDCl₃, 400 MHz) δ 11.53 (s, 1H), 8.50 (s, 1H), 7.99~8.03 (m, 3H), 7.66 (s, 1H), 7.50~7.52 (m, 2H), 7.38~7.45 (m, 4H), 7.07~7.10 (m, 1H), 6.95~7.01 (m, 3H), 3.87 (s, 3H), 3.08 (s, 3H), 3.01 (s, 3H), 2.81 (d, J = 4.8 Hz, 3H). | 598 |
| 603 | 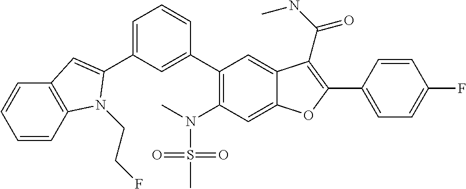 | ¹H-NMR (CDCl₃, 400 MHz) δ 7.94~7.95 (m, 2H), 7.85 (s, 1H), 7.39~7.67 (m, 7H), 7.15~7.24 (m, 4H), 6.62 (s, 1H), 5.86 (s, 1H), 4.68~4.70 (m, 1H), 4.54~4.58 (m, 1H), 4.48~4.51 (m, 1H), 4.09~4.15 (m, 1H), 3.21 (s, 3H), 2.99 (d, J = 4.8 Hz, 3H), 2.65 (s, 3H). | 614 |
| 604 | 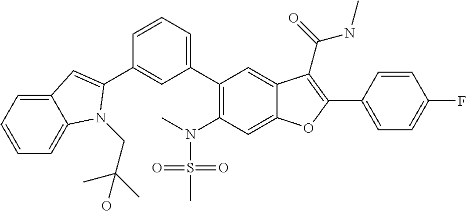 | ¹H-NMR (CDCl₃, 400 MHz) δ 7.83~7.87 (m, 4H), 7.75 (s, 1H), 7.70 (s, 1H), 7.35~7.46 (m, 6H), 7.14~7.16 (m, 4H), 6.54 (s, 1H), 4.35 (s, 2H), 3.21 (s, 3H), 2.90 (s, 3H), 2.73 (s, 3H), 0.92 (s, 6H). | 640 |
| 605 | 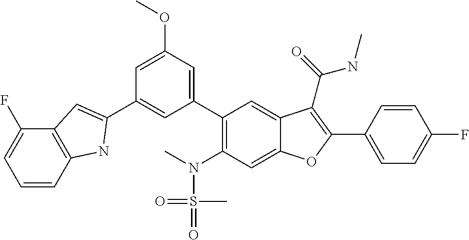 | ¹H-NMR (CDCl₃, 400 MHz) δ 9.32 (s, 1H), 7.94 (t, J = 4.6 Hz, 1H), 1.88 (d, J = 4.8 Hz, 1H), 7.51 (d, J = 5.2 Hz, 2H), 7.07~7.27 (m, 6H), 6.94 (s, 1H), 6.89 (s, 1H), 6.75~6.80 (m, 1H), 5.92 (s, 1H), 3.93 (d, J = 4.8 Hz, 3H), 3.07 (d, J = 4.8 Hz, 3H), 2.99 (d, J = 4.8 Hz, 3H), 2.96 (d, J = 4.8 Hz, 3H). | 616 |

### Example 606

### 5-(3(4-fluorobenzo[d]oxazol-2-yl)-4-hydroxyphenyl)-2(4-fluorophenyl)-N-methyl-6-(N-methylmethylsulfonamido)benzofuran-3-carboxamide

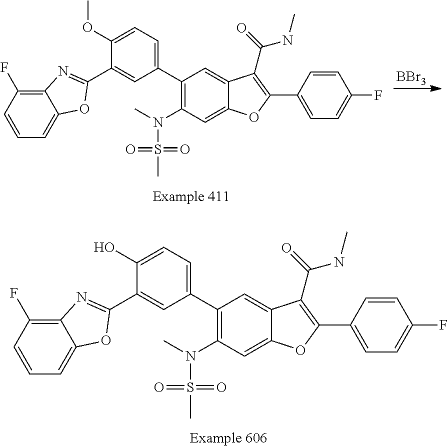

A solution of the compound of Example 411 (120 mg, 0.19 mmol) in anhydrous CH₂Cl₂ (3 mL), was cooled to −30° C. and a solution of BBr₃ (142 mg, 0.57 mmol) in dichloromethane was added dropwise. The reaction was allowed to stir at room temperature for 3 hours, then was quenched with water and extracted with CH₂Cl₂. The organic phase was dried over Na₂SO₄, filtered and concentrated in vacuo, and the residue obtained was purified using prep-TLC (petroleum ether:EtOAc=2:1) to provide the target compound (110 mg, 94%). ¹H -NMR (CDCl₃, 400 MHz) 8.18 (s, 1H), 7.94˜7.98 (m, 2H), 7.71 (d, J=2.4 Hz, 1H), 7.59 (s, 1H), 7.42˜7.43 (m, 2H), 7.33˜7.37 (m, 2H), 7.21˜7.25 (m, 2H), 7.14 (d, J=8.8 Hz, 1H), 5.87 (d, J=4.4 Hz, 1H), 4.02 (s, 3H), 3.12 (s, 3H), 3.01 (d, J=4.8 Hz, 3H). MS (M+H)⁺: 604.

The following compounds of the present invention were made using the method described above and using the appropriate reactants and/or reagents.

| | | | MS |
|---|---|---|---|
| Example | Structure | NMR | (M + H)⁺ |
| 607 | 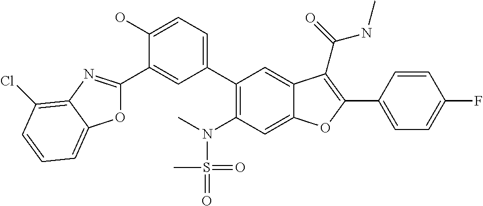 | ¹H-NMR (DMSO, 400 MHz) δ 11.03 (s, 1H), 8.02 (s, 1H), 7.94~7.91 (m, 2H), 7.90 (s, 1H), 7.85 (s, 1H), 7.56 (dd, J = 1.2 Hz, 1H), 7.41 (s, 1H), 7.35~7.32 (m, 1H), 7.21~7.17 (m, 3H), 7.14~7.09 (m, 1H), 5.88 (s, 1H), 3.16 (s, 3H), 2.97 (s, 3H), 2.73 (s, 3H). | 621 |
| 608 | 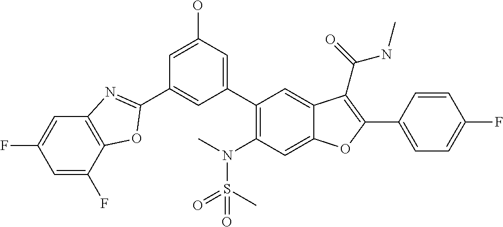 | ¹H-NMR (CDCl₃, 400 MHz) δ 7.79~7.82 (m, 2H), 7.73 (s, 1H), 7.64 (s, 1H), 7.59 (s, 1H), 7.51 (s, 1H), 7.04~7.14 (m, 4H), 6.84 (t, J = 9.6 Hz, 1H), 6.51 (s, 1H), 3.07 (s, 6H), 2.76 (s, 3H). | 622 |
| 609 | 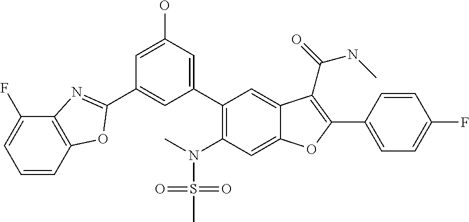 | ¹H-NMR (CDCl₃, 400 MHz) δ 7.98~8.02 (m, 2H), 7.85 (d, J = 2.0 Hz, 2H), 7.71 (d, J = 4.8 Hz, 2H), 7.54 (d, J = 8.4 Hz, 1H), 7.41~7.44 (m, 1H), 7.27~7.31 (m, 2H), 7.17~7.19 (m, 2H), 3.25 (s, 3H), 2.96 (s, 3H), 2.88 (s, 3H). | 604 |
| 610 | 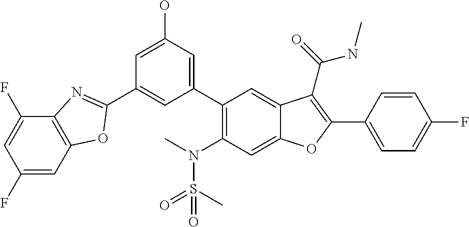 | ¹H-NMR (MeOD, 400 MHz): δ7.98~8.01 (m, 2H), 7.85 (s, 1H), 7.82 (s, 1H), 7.71 (s, 1H), 7.67 (s, 1H), 7.41 (d, J = 8.0 Hz, 1H), 7.28 (t, J = 8.8 Hz, 2H), 7.17 (s, 1H), 7.08 (t, J = 10.0 Hz, 1H), 7.81~7.85 (m, 1H), 7.67~7.71 (m, 1H), 3.24 (s, 3H), 2.95 (s, 3H), 2.88 (s, 3H). | 622 |
| 611 | 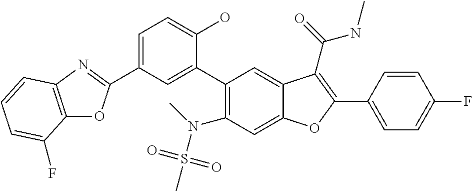 | ¹H-NMR (CDCl₃, 400 MHz) δ 8.24 (d, J = 1.6 Hz, 1H), 8.09~8.23 (m, 2H), 7.94~7.99 (m, 3H), 7.86 (s, 1H), 7.69 (s, 1H), 7.50 (d, J = 8.0 Hz, 1H), 7.06~7.22 (m, 3H), 5.91 (d, J = 1.2 Hz, 1H), 5.12 (s, 1H), 3.17 (s, 3H), 3.00 (d, J = 4.8 Hz, 3H), 2.82 (s, 3H). | 604 |
| 612 | 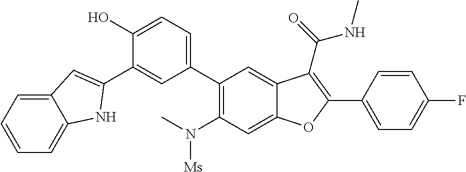 | ¹H-NMR (CDCl₃, 400 MHz) δ 9.30 (s, 1H), 8.75 (s, 1H), 7.79~8.30 (m, 2H), 7.54 (d, J = 7.6 Hz, 1H), 7.50 (s, 1H), 7.37 (d, J = 1.6 Hz, 1H), 6.91~7.19 (m, 7H), 6.65 (d, J = 1.2 Hz, 1H), 6.01 (d, J = 4.4 Hz, 1H), 2.95 (d, J = 4.8 Hz, 3H), 2.61 (s, 3H), 2.43 (s, 3H). | 584 |
| 613 | 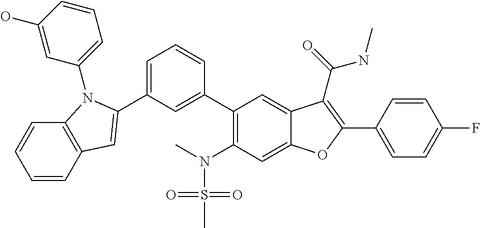 | ¹H-NMR (CDCl₃, 400 MHz) δ 7.79~7.82 (m, 2H), 7.61 (d, J = 6.8 Hz, 1H), 7.47 (d, J = 16.0 Hz, 2H), 7.35 (d, J = 8.0 Hz, 1H), 7.27 (d, J = 8.0 Hz, 2H), 7.21~7.25 (m, 2H), 7.10~7.17 (m, 5H), 6.93 (d, J = 8.0 Hz, 1H), 6.82 (d, J = 8.0 Hz, 1H), 6.74 (s, 1H), 6.55 (s, 1H), 5.85 (d, J = 4.0 Hz, 1H), 3.05 (s, 3H), 2.96 (d, J = 4.0 Hz, 3H), 2.63 (s, 3H). | 660 |
| 614 | 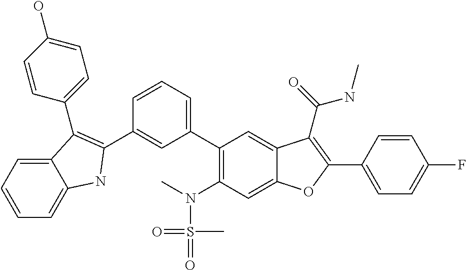 | ¹H-NMR (CDCl₃, 400 MHz) δ 9.00 (s, 1H), 7.82~7.86 (m, 2H), 7.68 (s. 1H), 7.64 (d, J = 8.0 Hz, 1H), 7.57 (s, 1H), 7.47 (s, 1H), 7.42~7.36 (m, 2H), 7.29~7.36 (m, 4H), 7.18 (t, J = 8.0 Hz, 3H), 7.11 (t, J = 8.0 Hz, 1H), 6.93 (d, J = 8.0 Hz, 2H), 5.99 (d, J = 4.0 Hz, 1H), 2.95 (s, 3H), 2.90 (s, 3H), 2.87 (s, 3H). | 660 |
| 615 | 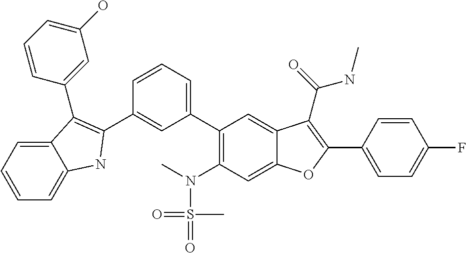 | ¹H-NMR (CDCl₃, 400 MHz) δ 9.17 (s, 1H), 7.75~7.78 (m, 2H), 7.70 (t, J = 4.0 Hz, 2H), 7.65 (s, 1H), 7.44 (d, J = 8.0 Hz, 1H), 7.38 (s, 1H), 7.33 (d, J = 7.6 Hz, 1H), 7.24~7.25 (m, 3H), 7.09~7.14 (m, 4H), 6.87 (s, 1H), 6.75~6.77 (m, 1H),. 6.48 (s, 1H), 6.14 (d, J = 3.6 Hz, 1H), 2.92 (s, 3H), 2.87 (s, 3H), 2.84 (d, J = 4.8 Hz, 3H). | 660 |

### Example 616

### 5-(4-(2,2-difluoroethoxy)-3-(4-fluorobenzoldloxazol-2-yl)phenyl)-2-(4-fluorophenyl)-N-methyl-6-(N-methylmethylsulfonamido) benzofuran-3-carboxamide

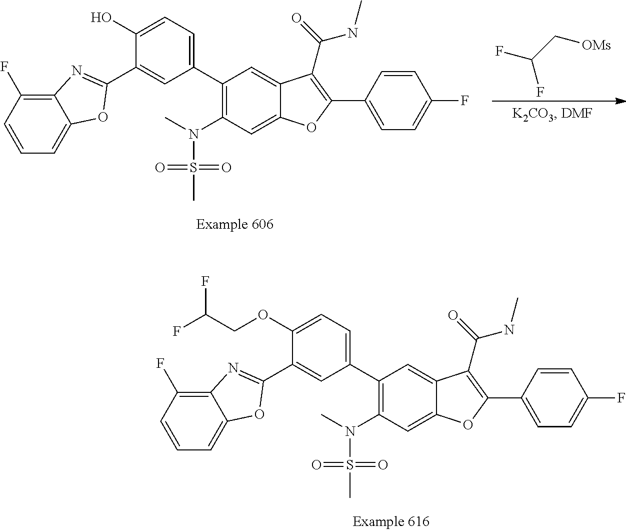

A solution of the compound of Example 606 (100 mg, 0.16 mmol), 2,2-difluoroethyl methanesulfonate (234 mg, 1.6 mmol) and K₂CO₃ (43 mg, 0.32 mmol) in DMF (3 mL) was heated to 100° C. and allowed to stir at this temperature for 3 hours. The reaction mixture was cooled to room temperature and filtered, and the filtrate was concentrated in vacuo. The residue obtained was purified using prep-HPLC to provide the target compound (30 mg, 25%). (CDCl₃, 400 MHz) 8.18 (s, 1H), 7.82˜7.85 (m, 2H), 7.76 (s, 1H), 7.59˜7.62 (m, 1H), 7.52 (s, 1H), 7.33 (d, J=8.0 Hz, 1H), 7.22˜7.27 (m, 1H), 7.06˜7.12 (m, 3H), 7.01 (t, J=1.8 Hz, 1H), 6.06˜6.38 (m, 2H), 4.32˜4.40 (m, 2H), 3.09 (s, 3H), 2.95 (d, J=4.8 Hz, 3H), 2.76 (s, 3H). MS (M+H)⁺: 668.

The following compounds of the present invention were made using the method described above and using the appropriate reactants and/or reagents.

| | | | MS |
|---|---|---|---|
| Example | Structure | NMR | (M + H)⁺ |
| 617 | 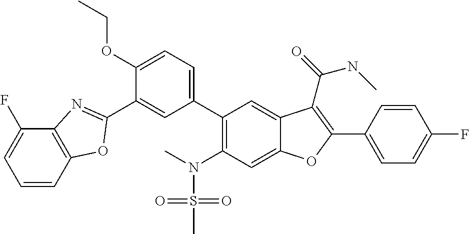 | ¹H-NMR (CDCl₃, 400 MHz) δ 8.14 (d, J = 2.0 Hz, 1H), 7.86~7.90 (m, 2H), 7.77 (s, 1H), 7.57~7.60 (m, 1H), 7.55 (s, 1H), 7.33 (d, J = 8.0 Hz, 1H), 7.22~7.26 (m, 1H), 7.05~7.15 (m, 3H), 7.01 (t, J = 8.8 Hz, 1H), 5.94 (d, J = 4.0 Hz, 1H), 4.20~4.25 (m, 2H), 3.09 (s, 3H), 2.95 (d, J = 5.2 Hz, 3H), 2.71 (s, 3H), 1.50 (t, J = 6.8 Hz, 3H). | 632 |
| 618 | 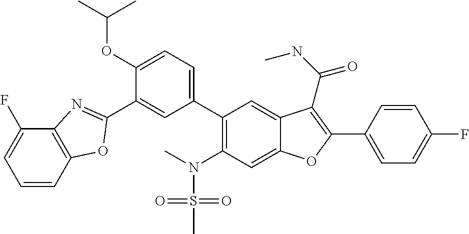 | ¹H-NMR (CDCl₃, 400 MHz) δ 8.19 (s, 1H), 7.89 (t, J = 5.6 Hz, 2H), 7.77 (s, 1H), 7.56~7.59 (m, 1H), 7.55 (s, 1H), 7.33 (d, J = 8.4 Hz, 1H), 7.21~7.26 (m, 1H), 7.09~7.15 (m, 3H), 7.00 (t, J = 8.4 Hz, 1H), 5.90 (d, J = 4.8 Hz, 1H), 4.69 (t, J = 6.0 Hz, 1H), 3.10 (s, 3H), 2.95 (d, J = 4.8 Hz, 3H), 2.71 (s, 3H), 1.42 (s, 3H), 1.41 (s, 3H). | 646 |
| 619 | 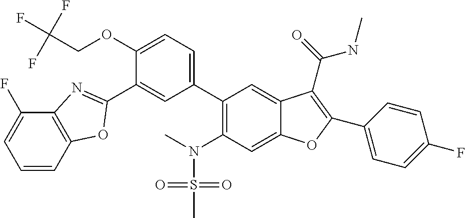 | ¹H-NMR (CDCl₃, 400 MHz) δ 8.21 (s, 1H), 7.85~7.88 (m, 1H), 7.79 (s, 1H), 7.63~7.66 (m, 1H), 7.54 (s, 1H), 7.34 (d, J = 8.4 Hz, 1H), 7.24~7.29 (m, 1H), 7.10~7.19 (m, 3H), 7.02 (t, J = 8.8 Hz, 1H), 5.97 (s, 1H), 4.52~4.58 (m, 2H), 3.08 (s, 3H), 2.95 (d, J = 4.8 Hz, 3H), 2.78 (s, 3H). | 686 |
| 620 | 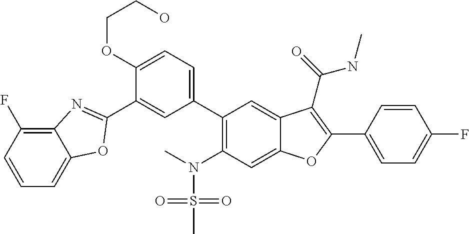 | ¹H-NMR (CDCl₃, 400 MHz) 8.20 (d, J = 2.0 Hz, 1H), 7.92~7.96 (m, 2H), 7.89 (s, 1H), 7.59~7.62 (m, 1H), 7.64 (s, 1H), 7.40~7.42 (m, 1H), 7.32~7.37 (m, 1H), 7.23~7.27 (m, 1H), 7.10~7.14 (m, 3H), 6.11 (d, J = 4.4 Hz, 1H), 4.32 (t, J = 4.4 Hz, 2H), 4.11 (t, J = 4.4 Hz, 2H), 3.18 (s, 3H), 3.02 (d, J = 4.8 Hz, 3H), 2.84 (s, 3H). | 648 |
| 621 | 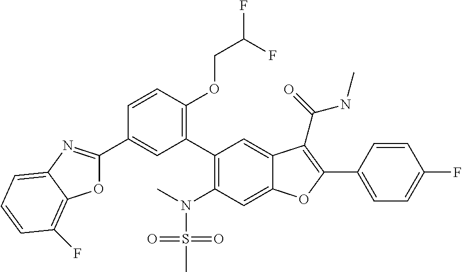 | ¹H-NMR (CDCl₃, 400 MHz) δ 8.24 (d, J = 2.0 Hz, 1H), 8.22 (d, J = 2.0 Hz, 1H), 7.88~8.17 (m, 2H), 7.72 (s, 1H), 7.54 (s, 1H), 7.43 (d, J = 6.4 Hz, 1H), 6.99~7.23 (m, 5H), 5.75~6.02 (m, 2H), 4.19 (d, J = 11.2 Hz, 2H), 3.14 (s, 3H), 2.91 (d, J = 4.8 Hz, 3H), 2.56 (s, 3H). | 668 |
| 622 | 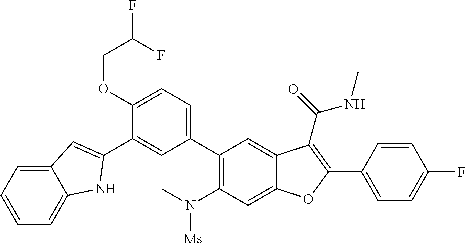 | ¹H-NMR (CDCl₃, 400 MHz) δ 9.30 (s, 1H), 7.92~8.94 (m, 3H), 7.91 (d, J = 2.0 Hz, 1H), 7.82 (s, 1H), 7.63 (d, J = 7.6 Hz, 1H), 7.35~7.56 (m, 2H), 7.02~7.21 (m, 5H), 6.96 (d, J = 1.6 Hz, 1H), 6.09~6.36 (m, 1H), 5.92 (d, J = 4.4 Hz, 1H), 4.34~4.42 (m, 2H), 3.06 (s, 3H), 2.97 (d, J = 4.8 Hz, 3H), 2.87 (s, 3H). | 648 |
| 623 | 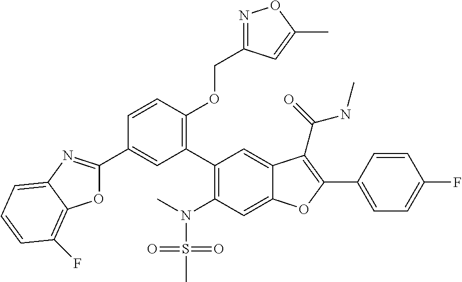 | ¹H-NMR (CDCl₃, 400 MHz) δ 8.11~8.25 (m, 2H), 7.87~7.96 (m, 2H), 7.74 (s, 1H), 7.54 (s, 1H), 7.42 (m, 1H), 7.08~7.24 (m, 4H), 7.00 (t, J = 8.8 Hz, 1H), 6.17 (s, 1H), 5.85 (s, 1H), 5.10 (s, 2H), 3.12 (s, 3H), 2.93 (d, J = 4.4 Hz, 3H), 2.51 (s, 3H), 2.29 (s, 3H). | 699 |
| 624 | 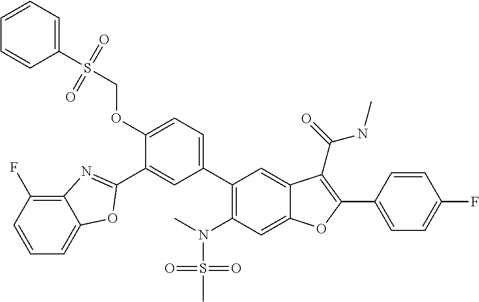 | ¹H-NMR (CDCl₃, 400 MHz) 8.32 (d, J = 2.0 Hz, 1H), 7.88~7.91 (m, 2H), 7.82 (s, 1H), 7.55~7.60 (m, 4H), 7.49~7.51 (m, 1H), 7.28~7.37 (m, 5H), 7.13~7.17 (m, 2H), 7.04~7.09 (m, 1H), 5.76 (t, J = 4.8 Hz, 1H), 5.01 (s, 2H), 3.14 (s, 3H), 2.94 (d, J = 4.8 Hz, 3H), 2.64 (s, 3H). | 758 |
| 625 | 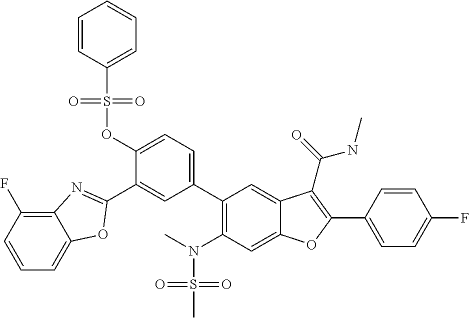 | ¹H-NMR (CDCl₃, 400 MHz) 8.19 (d, J = 2.0 Hz, 1H), 7.78~7.90 (m, 5H), 7.61~7.63 (m, 1H), 7.57 (s, 1H), 7.43~7.48 (m, 2H), 7.28~7.31 (m, 4H), 7.13~7.18 (m, 2H), 7.00~7.04 (m, 1H), 5.76 (t, J = 4.8 Hz, 1H), 3.13 (s, 3H), 2.95 (d, J = 4.8 Hz, 3H), 2.70 (s, 3H). | 744 |
| 626 | 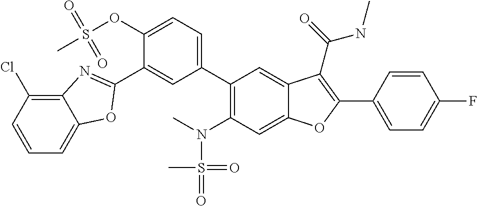 | ¹H-NMR (DMSO, 400 MHz) δ 8.31 (s, 1H), 7.88 (t, J = 5.6 Hz, 2H), 7.82 (s, 1H), 7.57~7.62 (m, 3H), 7.45 (d, J = 7.6 Hz, 1H), 7.33 (d, J = 7.6 Hz, 1H), 7.17~7.29 (m, 1H), 7.12~7.15 (m, 2H), 5.84 (s, 1H), 3.57 (s, 3H). 3.13 (s, 3H), 2.93 (s, 3H), 2.66 (s, 3H). | 699 |
| 627 | 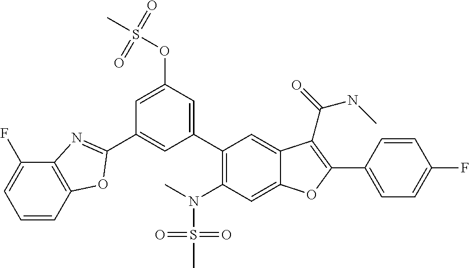 | ¹H-NMR (CDCl₃, 400 MHz) δ 8.33 (s, 1H), 8.23 (d, J = 12.8 Hz, 1H), 7.92~7.98 (m, 3H), 7.65~7.67 (m, 1H), 7.63 (s, 1H), 7.42~7.45 (m, 1H), 7.35~7.40 (m, 1H), 7.20~7.22 (m, 2H), 7.11~7.18 (m, 1H), 6.19 (d, J = 4.8 Hz, 1H), 3.31 (s, 3H), 3.23 (s, 3H), 3.06 (d, J = 4.8 Hz, 3H), 2.83 (s, 3H). | 682 |
| 628 | 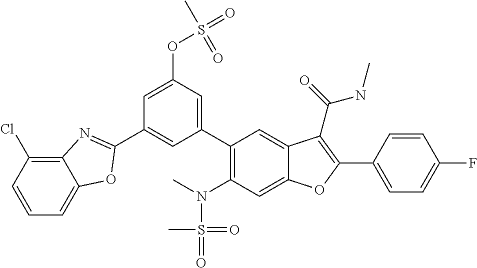 | ¹H-NMR (DMSO, 400 MHz) δ 8.30 (s, 1H), 8.16 (d, J = 1.6 Hz, 1H), 7.87~7.90 (m, 3H), 7.57~7.58 (m, 2H), 7.47 (d, J = 0.8 Hz, 1H), 7.45 (d, J = 0.8 Hz, 1H), 7.24~7.35 (m, 1H), 7.13~7.15 (m, 2H), 5.84 (s, 1H), 3.22 (s, 3H), 3.16 (s, 3H), 2.94 (s, 3H), 2.73 (s, 3H). | 699 |
| 629 | 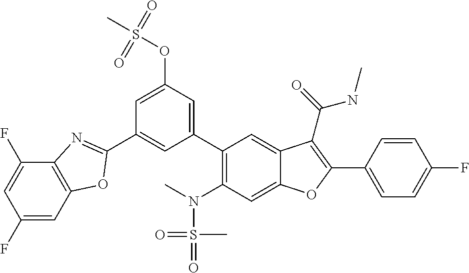 | ¹H-NMR (CDCl₃, 400 MHz) δ 8.28 (s, 1H), 8.15 (s, 1H), 7.90~7.93 (m, 3H), 7.63 (s, 1H), 7.61 (s, 1H), 7.17~7.22 (m, 3H), 6.92 (t, J = 9.8 Hz, 1H), 6.06 (d, J = 4.4 Hz, 1H), 3.28 (s, 3H), 3.22 (s, 3H), 3.02 (d, J = 4.8 Hz, 3H), 2.81 (s, 3H). | 700 |
| 630 | 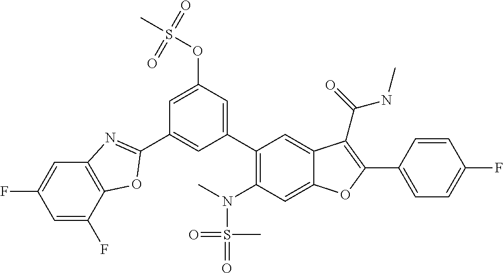 | ¹H-NMR (CDCl₃, 400 MHz) δ 8.29 (s, 1H), 8.18 (s, 1H), 7.92~7.95 (m, 3H), 7.66 (s, 1H), 7.63 (s, 1H), 7.28 (d, J = 8.0 Hz, 1H), 7.22 (t, J = 8.4 Hz, 2H), 6.95 (t, J = 9.6 Hz, 1H), 5.94 (d, J = 4.0 Hz, 1H), 3.28 (s, 3H), 3.24 (s, 3H), 3.01 (d, J = 4.8 Hz, 3H), 2.80 (s, 3H). | 700 |

### Example 631

### 2-{5-[2-(4-Fluoro-phenyl)-6-(methanesulfonyl-methylamino)-3-methylcarbamoyl-benzofuran-5-yl]-2-methoxy-phenyl}-benzooxazole-5-carboxylic acid methylamide

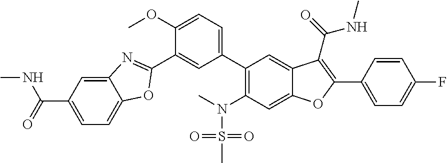

### Step 1—Synthesis of 2-{5-[2-(4-Fluoro-phenyl)-6-(methanesulfonyl-methyl-amino)-3-methylcarbamoyl-benzofuran-5-yl]-2-methoxy-phenyl}-benzooxazole-5-carboxylic acid methyl ester

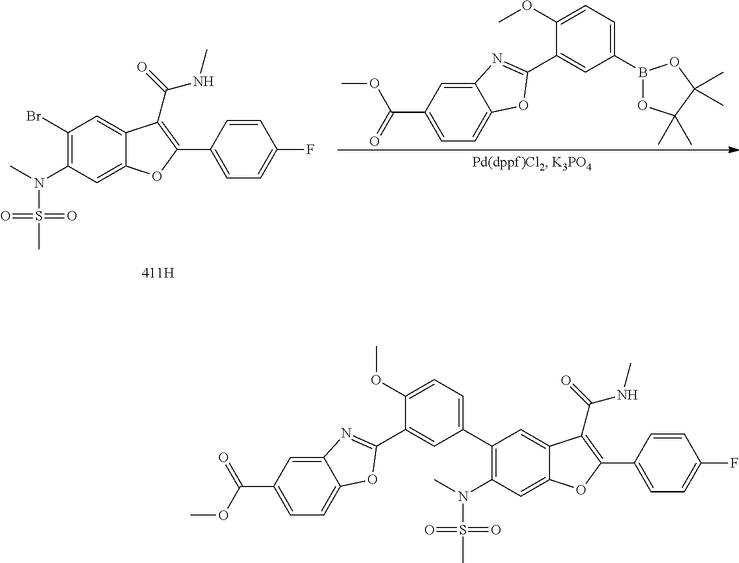

Compound 411H was converted to methyl 2-(5-(2-(4-fluorophenyl)-3-(methylcarbamoyl)-6-(N-methylmethylsulfonamido) benzofuran-5-yl)-2-methoxyphenyl)benzo[d]oxazole-5-carboxylate (95 mg, 58.3%) using the method described in Example 411, Step 12. ¹H-NMR (DMSO-d₆, 400 MHz) δ 8.51 (d, J=3.6 Hz, 1H), 8.33 (s, 1H), 7.97˜8.09 (m, 4H), 7.90 (t, J=8.0 Hz, 1H), 7.70 (d, J=6.0 Hz, 1H), 7.61 (s, 1H), 7.35˜7.41 (m, 3H), 3.98 (s, 3H), 3.87 (s, 3H), 3.11 (s, 3H), 2.98 (s, 3H), 2.78 (d, J=4.4 Hz, 3H). MS (M+H)⁺: 658.

### Step 2—Synthesis of 2-{5-[2-(4-Fluoro-phenyl)-6-(methanesulfonyl-methyl-amino)-3-methylcarbamoyl-benzofuran-5-yl]-2-methoxy-phenyl}-benzooxazole-5-carboxylic acid

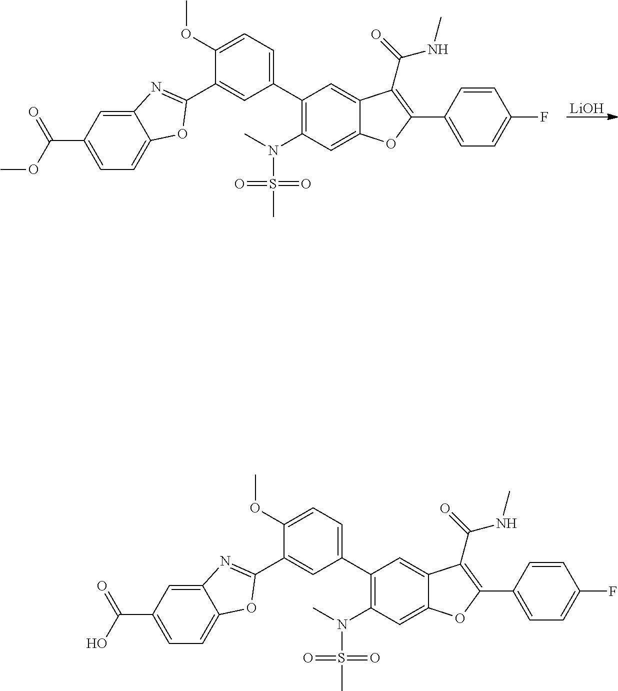

Methyl-2-(5-(2-(4-fluorophenyl)-3-(methylcarbamoyl)-6-(N-methylmethylsulfonamido)benzofuran-5-yl)-2-methoxyphenyl)benzo[d]oxazole-5-carboxylate was converted to 2-(5-(2-(4-fluorophenyl)-3-(methylcarbamoyl)-6-(N-methylmethylsulfonamido)benzofuran-5-yl)-2-methoxyphenyl)benzo[d]oxazole-5-carboxylic acid (85 mg, 100%) using the method described in Example 411, Step 4. ¹H-NMR (MeOD, 400 MHz) δ 8.03 (d, J=7.2 Hz, 1H), 7.87˜7.90 (m, 2H), 7.59˜7.74 (m, 6H), 7.25 (d, J=8.8 Hz, 1H), 7.12˜7.17 (m, 3H), 3.98 (s, 3H), 3.11 (s, 3H), 2.86 (s, 3H), 2.83 (s, 3H). MS (M+H)⁺: 644.

### Step 3—Synthesis of 2-{5-[2-(4-Fluoro-phenyl)-6-(methanesulfonyl-methyl-amino)-3-methylcarbamoyl-benzofuran-5-yl]-2-methoxy-phenyl}-benzooxazole-5-carboxylic acid methylamide

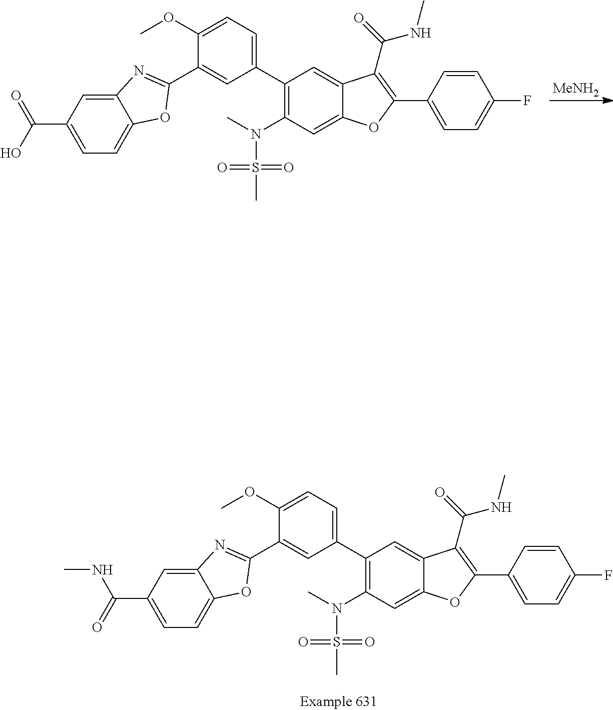

2-(5-(2-(4-fluorophenyl)-3-(methylcarbamoyl)-6-(N-methylmethylsulfonamido)benzofuran-5-yl)-2-methoxyphenyl)benzo[d]oxazole-5-carboxylic acid was converted to the title compound (35 mg, 35.8%) using the method described in Example 411, Step 5. ¹H-NMR (CDCl₃, 400 MHz) δ 8.15 (s, 1H), 8.08 (s, 1H), 7.87˜7.90 (m, 2H), 7.81 (s, 1H), 7.88 (s, 1H), 7.63˜7.54 (m, 1H), 7.56 (t, J=4.0 Hz, 2H), 7.12˜7.17 (m, 3H), 6.11 (br s, 1H), 5.80 (d, J=4.8 Hz, 1H), 4.03 (s, 3H), 3.10 (s, 3H), 2.99 (d, J=4.8 Hz, 3H), 2.93 (d, J=4.8 Hz, 3H), 2.73 (s, 3H). MS (M+H)⁺: 657.

The following compounds of the present invention were made using the method described above and using the appropriate reactants and/or reagents.

| | | | MS |
|---|---|---|---|
| Example | Structure | NMR | (M + H)⁺ |
| 632 | 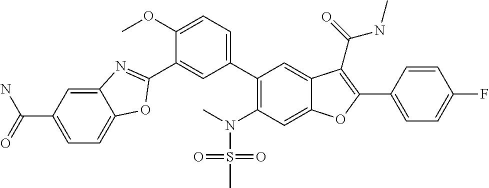 | ¹H-NMR (MeOD, 400 MHz) δ 8.20 (s, 1H), 8.15 (d, J = 2.4 Hz, 1H), 7.87~7.91 (m, 3H), 7.81 (s, 1H), 7.63~7.68 (m, 3H), 7.16~7.27 (m, 3H), 3.99 (s, 3H), 3.11 (s, 3H), 2.81~2.88 (m, 6H). | 643 |
| 633 | 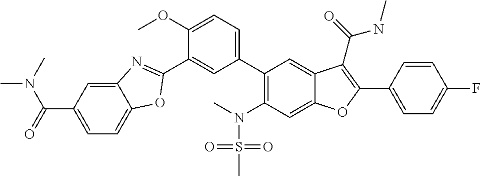 | ¹H-NMR (CDCl₃, 400 MHz) δ 8.16 (s, 1H), 7.81~7.90 (m, 2H), 7.81 (s, 2H), 7.64 (d, J = 8.4 Hz, 1H), 7.56 (t, J = 4.0 Hz, 2H), 7.38~7.43 (m, 1H), 7.15 (t, J = 8.8 Hz, 3H), 5.81 (d, J = 3.6 Hz, 1H), 4.02 (s, 3H), 3.10 (s, 6H), 2.98 (s, 3H), 2.93 (d, J = 4.8 Hz, 3H), 2.74 (s, 3H). | 671 |
| 634 | 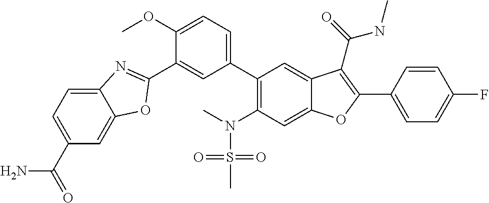 | ¹H-NMR (CDCl₃, 400 MHz) δ 8.18 (d, J = 2.0 Hz, 1H), 8.06 (s, 1H), 7.72~7.91 (m, 5H), 7.63~7.66 (m, 1H), 7.53 (s, 1H), 7.09~7.22 (m, 3H), 6.20~6.50 (br, 2H), 5.86 (d, J = 5.2 Hz, 1H), 4.02 (s, 3H), 3.11 (s, 3H), 2.94 (d, J = 4.8 Hz, 3H), 2.75 (s, 3H). | 643 |
| 635 | 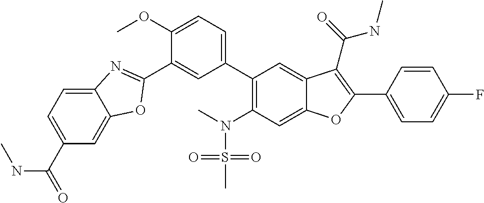 | ¹H-NMR (CDCl₃, 400 MHz) δ 8.11 (d, J = 2.0 Hz, 1H), 7.96 (s, 1H), 7.81~7.85 (m, 2H), 7.74~7.78 (m, 2H), 7.63~7.66 (m, 2H), 7.53 (s, 1H), 7.09~7.16 (m, 3H), 6.53 (s, 1H), 6.21 (s, 1H), 5.30~5.70 (br s, 2H), 3.98 (s, 3H), 3.09 (s, 3H), 2.96 (s, 3H), 2.94 (d, J = 4.8 Hz, 3H), 2.77 (s, 3H). | 657 |
| 636 | 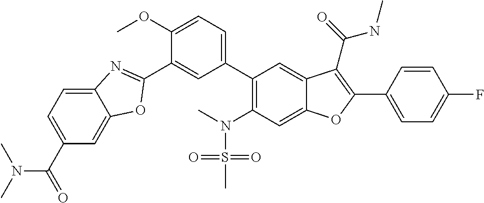 | ¹H-NMR (CDCl₃, 400 MHz) δ 8.18 (s, 1H), 7.88~7.93 (m, 3H), 7.84 (s, 1H), 7.69~7.73 (m, 2H), 7.61 (s, 1H), 7.43~7.45 (m, 1H), 7.15~7.22 (m, 3H), 6.04 (d, J = 4.0 Hz, 1H), 4.06 (s, 3H), 3.17 (s, 6H), 3.05 (s, 3H), 2.99 (d, J = 5.2 Hz, 3H), 2.80 (s, 3H). | 671 |
| 637 | 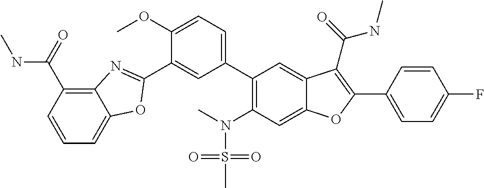 | ¹H-NMR (CDCl₃, 400 MHz) δ 9.25 (s, 1H), 8.36 (s, 1H), 8.17 (d, J = 7.2 Hz, 1H), 7.89~7.93 (m, 3H), 7.66~7.72 (m, 2H), 7.61 (s, 1H), 7.43 (t, J = 8.0 Hz, 1H), 7.16~7.22 (m, 3H), 5.91 (d, J = 4.8 Hz, 1H), 4.07 (s, 3H), 3.11 (s, 6H), 2.97 (d, J = 4.8 Hz, 3H), 2.86 (s, 3H). | 657 |
| 638 | 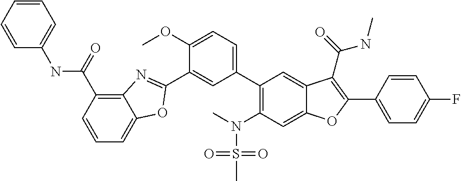 | ¹H-NMR (CDCl₃, 400 MHz) δ 11.44 (s, 1H), 8.29 (d, J = 2.0 Hz, 1H), 8.24 (d, J = 7.6 Hz, 1H), 7.90-7.94 (m, 3H), 7.84 (d, J = 8.0 Hz, 2H), 7.70~7.75 (m, 2H), 7.60 (s, 1H), 7.49 (t, J = 8.0 Hz, 1H), 7.38 (t, J = 8.0 Hz, 2H), 7.18~7.23 (m, 3H), 7.13 (t, J = 7.2 Hz, 1H), 5.96 (s, 1H), 4.13 (s, 3H), 3.15 (s, 3H), 2.97 (d, J = 4.8 Hz, 3H), 2.79 (s, 3H). | 719 |

### Example 639

### 2-(4-fluorophenyl)-5-(4-methoxy-3-(4-(methylsulfonamido)benzo[d]oxazol-2-yl)phenyl)-N-methyl-6-(N-methylmethylsulfonamido)benzofuran-3-carboxamide

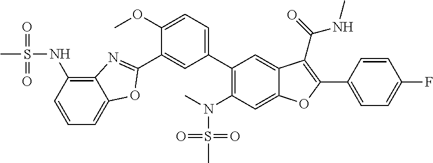

### Step 1—Synthesis of 2-(4-fluorophenyl)-5-(4-methoxy-3-(4-nitrobenzo[d]oxazol-2-yl)phenyl)-N-methyl-6-(N-methylmethylsulfonamido)benzofuran-3-carboxamide

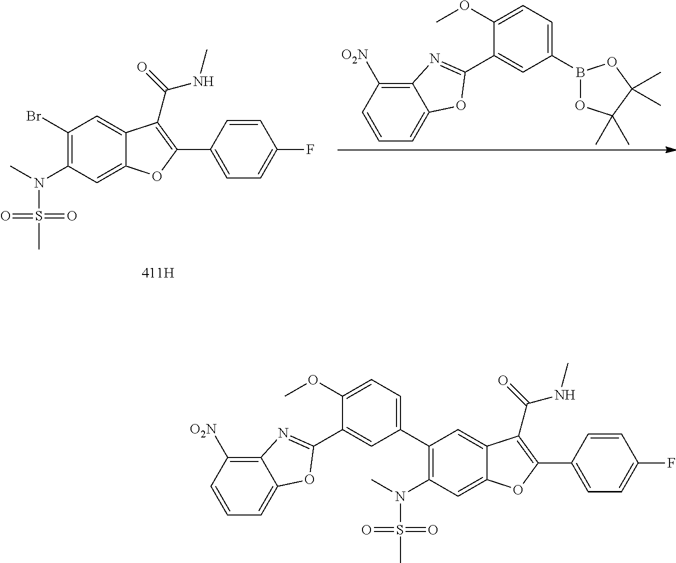

Compound 411H was converted to 2-(4-fluorophenyl)-5-(4-methoxy-3-(4 nitrobenzo[d]oxazol-2-yl)phenyl)-N-methyl-6-(N-methylmethylsulfonamido)benzofuran-3-carboxamide (2.5 g, 86%) using the method described in Example 411, Step 12. ¹H-NMR (CDCl₃, 400 MHz) δ 8.36˜8.37 (m, 1H), 7.93˜7.96 (m, 2H), 7.84 (s, 1H), 7.63˜7.67 (m, 1H), 7.61 (s, 1H), 7.32˜7.35 (m, 2H), 7.23˜7.31 (m, 1H), 7.17˜7.21 (m, 2H), 6.83 (d, J=7.6 Hz, 1H), 6.05 (d, J=4.8 Hz, 1H), 4.05 (s, 3H), 3.14 (s, 3H), 3.02 (d, J=4.8 Hz, 3H), 2.80 (s, 3H).

### Step 2—Synthesis of 5-(3-(4-aminobenzo[d]oxazol-2-yl)-4-methoxyphenyl)-2-(4-fluorophenyl)-N-methyl-6-(N-methylmethylsulfonamido)benzofuran-3-carboxamide

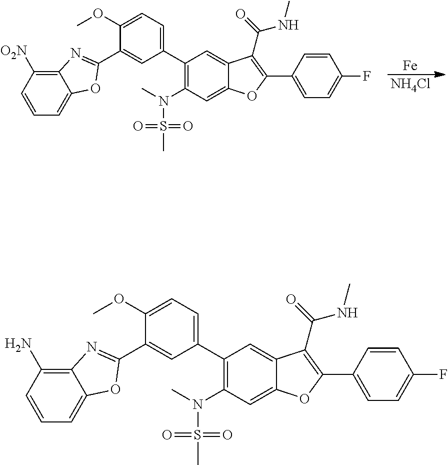

A mixture of 2-(4-fluorophenyl)-5-(4-methoxy-3-(4-nitrobenzo[d]oxazol-2-yl)phenyl)-N-methyl-6-(N-methylmethylsulfonamido)benzofuran-3-carboxamide (2.5 g, 3.88 mmol), Fe (0.7 g, 12.5 mmol) and NH₄Cl (1 g, 19.4 mmol) in MeOH (10 mL) and H₂O (10 mL) THF (5 mL) was allowed to stir at reflux for 3 hours. After being filtered and concentrated in vacuo, the residue obtained was purified using column chromatography eluted with petroleum ether:EtOAc=1:1 to provide 5-(3-(4-aminobenzo[d]oxazol-2-yl)-4-methoxyphenyl)-2-(4-fluorophenyl)-N-methyl-6-(N-methylmethylsulfonamido)benzofuran-3-carboxamide (1.7 g, 72%). ¹H-NMR (CDCl₃, 400 MHz) δ 8.05 (s, 1H), 7.70˜7.78 (m, 2H), 7.48˜7.52 (m, 2H), 7.01˜7.12 (m, 4′-1), 6.83˜6.88 (m, 1H), 6.48˜6.53 (m, 1H), 6.02˜6.04 (m 1H), 5.25 (s, 1H), 4.05 (s, 3H), 3.14 (s, 3H), 2.70 (m, 3H), 2.65 (s, 3H). MS (M+H)⁺: 615.

### Step 3—Synthesis of 2-(4-fluorophenyl)-5-(4-methoxy-3-(4-(methylsulfonamido) benzo[d]oxazol-2-yl)phenyl)-N-methyl-6-(N-methylmethylsulfonamido)benzofuran-3-carboxamide

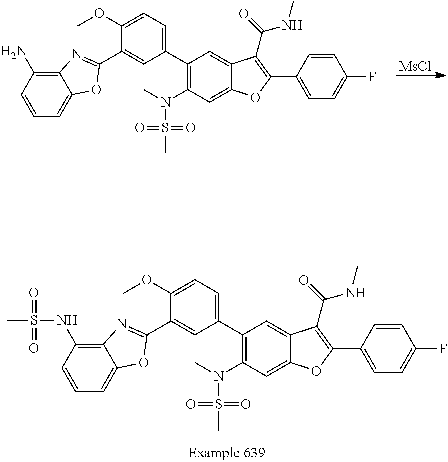

5-(3-(4-aminobenzo[d]oxazol-2-yl)-4-methoxyphenyl)-2-(4-fluorophenyl)-N-methyl-6-(N-methylmethylsulfonamido)benzofuran-3-carboxamide was converted to the title compound (30 mg, 20%) using the method described in Example 411, Step 3. ¹H-NMR (CDCl₃, 400 MHz) δ 8.21 (d, J=2.4 Hz, 1H), 7.99 (s, 1H), 7.91˜7.95 (m, 2H), 7.89 (s, 1H), 7.67 (d, J=8.8 Hz, 1H), 7.61 (s, 1H), 7.48 (d, J=7.2 Hz, 1H), 7.26˜7.35 (m, 2H), 7.17˜7.23 (m, 3H), 5.93 (d, J=4.8 Hz, 1H), 4.07 (s, 3H), 3.16 (s, 3H), 3.12 (s, 3H), 2.99 (d, J=5.2 Hz, 3H), 2.00 (s, 3H).

MS (M+H)⁺: 693.

### Example 640

### 2-(4-fluorophenyl)-5-(3-(4-(furan-2-yl)benzo[d]oxazol-2-yl)-4-methoxyphenyl)-N-methyl-6-(N-methylmethylsulfonamido)benzofuran-3-carboxamide

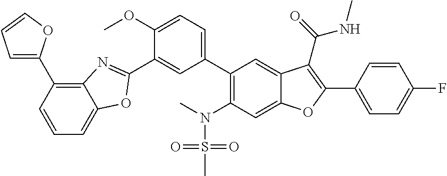

### Step 1—Synthesis of 2-(4-fluorophenyl)-5-(3-(4-iodobenzo[d]oxazol-2-yl)-4-methoxyphenyl)-N-methyl-6-(N-methylmethylsulfonamido) benzofuran-3-carboxamide

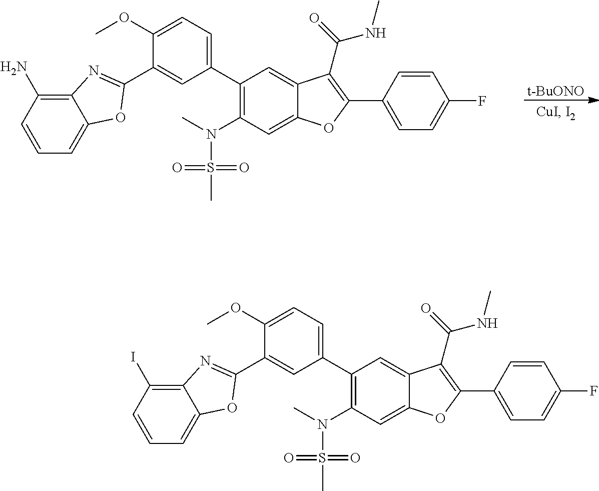

A mixture of 5-(3-(4-aminobenzo[d]oxazol-2-yl)-4-methoxyphenyl)-2-(4-fluorophenyl)-N-methyl-6-(N-methylmethylsulfonamido)benzofuran-3-carboxamide (1.5 g, 2.44 mmol), CuI (0.8 g, 4.2 mmol) and I₂ (0.5 g, 1.97 mmol) in CH₃CN (10 mL) was allowed to stir at 30° C. and allowed to stir at this temperature for 30 minutes. Then the reaction was cooled to 0° C. and isopentyl nitrite (0.6 g, 5.12 mmol) was added at 0° C. and the reaction was allowed to warm to 30° C. and stir at this temperature for about 15 hours. After being filtered and concentrated in vacuo, the residue obtained was purified using column chromatography eluted with petroleum ether:EtOAc=2:1 to provide 2-(4-fluorophenyl)-5-(3-(4-iodobenzo[d]oxazol-2-yl)-4-methoxyphenyl): N-methyl-6-(N-methylmethylsulfonamido)benzofuran-3-carboxamide (0.7 g, 40%). ¹H-NMR (CDCl₃, 400 MHz) δ 8.18 (s, 1H), 7.93˜7.96 (m, 2H), 7.84 (s, 1H), 7.63˜7.67 (m, 1H), 7.61 (s, 1H), 7.32˜7.35 (m, 1H), 7.23˜7.31 (m, 1H), 7.02˜7.19 (m, 4H), 5.85 (s, 1H), 4.00 (s, 3H), 3.10 (s, 3H), 3.93˜3.95 (m, 3H), 2.77 (s, 3H). MS (M+H)⁺: 726.

### Step 2—Synthesis of 2-(4-fluorophenyl)-5-(3-(4-(furan-2-yl)benzo[d]oxazol-2-yl)-4-methoxyphenyl)-N-methyl-6-(N-methylmethylsulfonamido)benzofuran-3-carboxamide

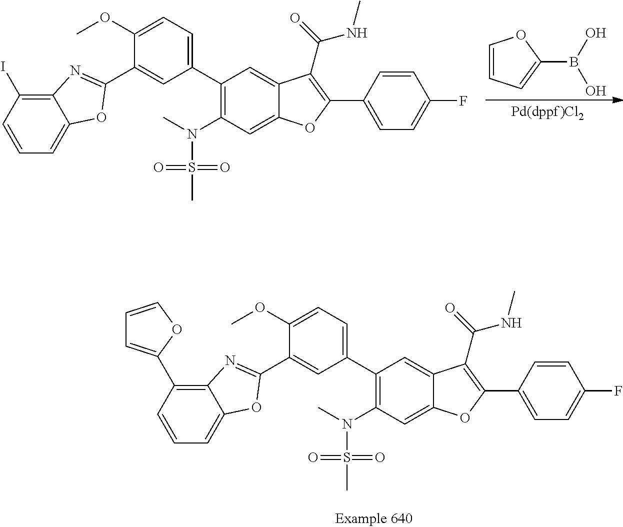

2-(4-fluorophenyl)-5-(3-(4-iodobenzo[d]oxazol-2-yl)-4-methoxyphenyl)-N-methyl-6-(N-methylmethylsulfonamido)benzofuran-3-carboxamide was converted to the title compound (40 mg, 44%) using the method described in Example 411, Step 12. ¹H-NMR (CDCl₃, 400 MHz) δ 8.35 (s, 1H), 7.87˜7.88 (m, 2H), 7.80 (s, 1H), 7.61˜7.70 (m, 1H), 7.57˜7.60 (m, 2H), 7.48˜7.49 (m, 2H), 7.47˜7.48 (m, 1H), 7.28˜7.31 (m, 1H), 7.09˜7.13 (m, 3H), 6.50˜6.51 (m, 1H), 5.84 (d, J=4.8 Hz, 1H), 4.03 (s, 3H), 3.11 (s, 3H), 2.92 (d, J=5.2 Hz, 3H), 2.70 (s, 3H). MS (M+H)⁺: 666.

The following compound of the present invention was made using the method described above and using the appropriate reactants and/or reagents.

| | | | MS |
|---|---|---|---|
| Example | Structure | NMR | (M + H)⁺ |
| 641 | 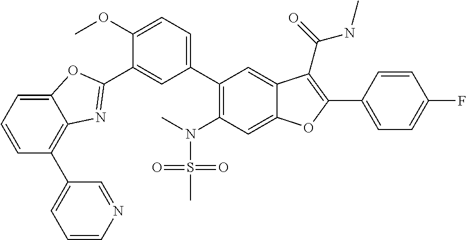 | ¹H-NMR (CDCl₃) 400 MHz) δ 9.21 (s 1H), 8.51 (s, 1H), 8.37~8.39 (m, 1H), 8.20 (s, 1H), 7.81~7.89 (m, 2H), 7.80 (s, 1H), 7.57~7.60 (m, 3H), 7.48~7.51 (m, 1H), 7.47~7.50 (m, 2H), 7.03~7.10 (m, 3H), 6.08 (s, 1H), 3.99 (s, 3H), 3.08 (s, 3H), 2.89 (d, J = 4.8 Hz, 3H), 2.71 (s, 3H). | 677 |

### Example 642

### (Z)-5-(3-(4-fluorobenzo[d]oxazol-2-yl)-5-(1-(hydroxyimino)ethyl)phenyl)-2-(4-fluorophenyl)-N-methyl-6-(N-methylmethylsulfonamido) benzofuran-3-carboxamide

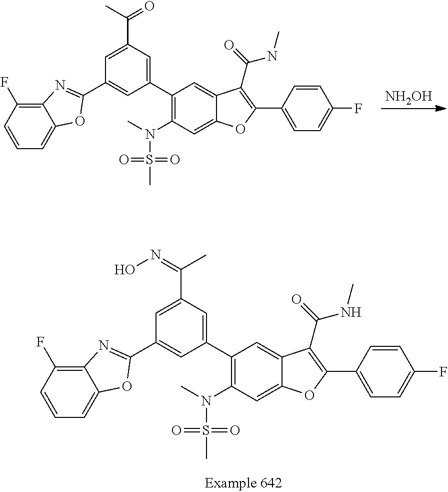

A mixture of 5-(3-acetyl-5-(4-fluorobenzo[d]oxazol-2-yl)phenyl)-2-(4-fluorophenyl)-N-methyl-6-(N-methylmethylsulfonamido)benzofuran-3-carboxamide (120 mg, 0.19 mmol, prepared according to the method described in Example 1), NH₂OH.HCl (27 mg, 0.38 mmol) and NaHCO₃ (32 mg, 0.38 mmol) in CH₃OH (5 mL) was heated to 50° C. and allowed to stir at this temperature for 5 hours. After the reaction mixture was cooled room temperature and concentrated in vacuo, the residue obtained was washed with CH₂Cl₂ and filtered. The filtrate was dried over Na₂SO₄, concentrated in vacuo, and the residue obtained was purified using column chromatography eluted with petroleum ether:EtOAc=2:1 to provide the title compound (100 mg, 80%). ¹H-NMR (CDCl₃, 400 MHz) 8.46 (s, 1H), 8.27 (s, 1H), 7.83˜7.92 (m, 4H), 7.57 (s, 1H), 7.34˜7.36 (m, 1H), 7.23˜7.29 (m, 3H), 7.00˜7.05 (m, 1H), 5.98 (s, 1H), 3.06 (s, 3H), 2.94 (t, J=4.8 Hz, 3H), 2.70 (s, 3H), 2.32 (s, 3H). MS (M+H)⁺: 645.

### Example 643

### 5-(3-(1-aminoethyl)-5-(4-fluorobenzo[d]oxazol-2-yl)phenyl)-2-(4-fluorophenyl)-N-methyl-6-(N-methylmethylsulfonamido)benzofuran-3-carboxamide

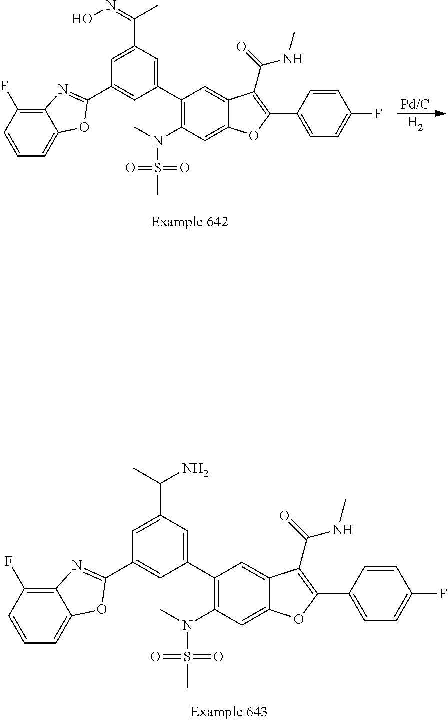

To a solution of the compound of Example 642 (40 mg, 0.06 mmol) in CH₃OH (3 mL) was added Pd/C (10 mg) and HCl (1N, 2 drops) and the resulting reaction was put under H₂ atmosphere (1 atm) and stirred for 12 hours. The reaction mixture was filtered and the filtrate was concentrated in vacuo to provide the title compound (30 mg, 75%). ¹H-NMR (CDCl₃, 400 MHz) 8.15 (s, 2H), 7.82˜7.85 (m, 2H), 7.76 (s, 1H), 7.69 (s, 1H), 7.37 (s, 1H), 7.23˜7.28 (m, 1H), 7.11˜7.15 (m, 3H), 7.02 (t, J=8.4 Hz, 1H), 6.44 (d, J=4.0 Hz, 1H), 4.48˜4.51 (m, 1H), 3.02 (s, 3H), 2.89 (d, J=4.4 Hz, 3H), 2.75 (s, 3H), 1.67 (d, J=7.2 Hz, 3H). MS (M+H)⁺: 631.

### Example 644

### 2-(4-fluorophenyl)-N-methyl-6-(N-methylmethylsulfonamido)-5-(3-(4,5,6,7-tetrahydrofuro[3,2-c]pyridin-2-yl)phenyl)benzofuran-3-carboxamide

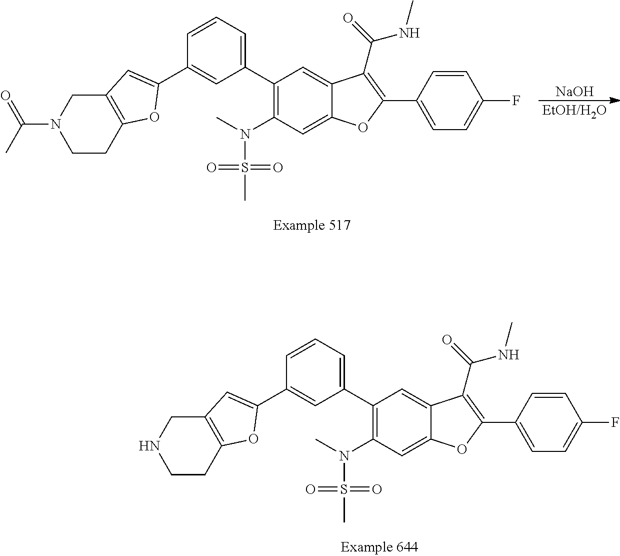

A mixture of the compound of Example 517 (166 mg, 0.27 mmol) and NaOH (108 mg, 2.7 mmol) in 9 mL of EtOH:H₂O (2:1) was heated to 90° C. under N₂ and allowed to stir at this temperature for about 15 hours. Then the reaction mixture was mixture was purified using prep-HPLC to provide the title compound (76 mg, 49.0%). ¹H-NMR (DMSO, 400 MHz) δ 9.10 (s, 1H), 8.50˜8.54 (m, 1H), 7.97˜8.00 (m, 3H), 7.74 (s, 1H), 7.68 (d, J=7.6 Hz, 1H), 7.58 (s, 1H), 7.49 (t, J=7.6 Hz, 1H), 7.37˜7.43 (m, 3H), 6.92 (s, 1H), 4.12 (s, 2H), 3.46 (s, 2H), 3.08 (s, 3H), 2.97 (s, 2H), 2.94 (s, 3H), 2.80 (d, J=4.8 Hz, 3H). MS (M+H)⁺: 574.

### Example 645

### 5-(3-(4-fluorobenzo[d]oxazol-2-yl)-4-(methylsulfonamido)phenyl)-2-(4-fluorophenyl)-N-methyl-6-(N-methylmethylsulfonamido)benzofuran-3-carboxamide

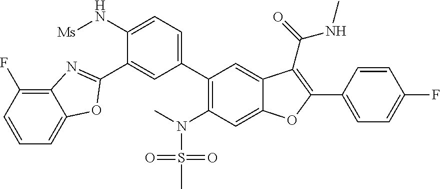

### Step 1—Synthesis of 5-(3-(4-fluorobenzo[d]oxazol-2-yl)-4-nitrophenyl)-2-(4-fluorophenyl)-N-methyl-6-(N-methylmethylsulfonamido)benzofuran-3-carboxamide

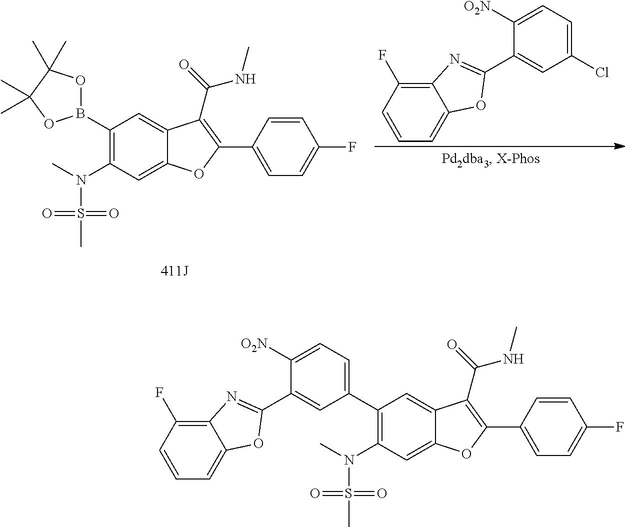

To a degassed solution of 2-(5-chloro-2-nitrophenyl)-4-fluorobenzo[d]oxazole (39 mg, 0.13 mmol) and Compound 411J (50 mg, 0.1 mmol) in 1,4-dioxane (2.0 mL) was added Pd₂(dba)₃ (5 mg), X-Phos (5 mg) and K₃PO₄ (42 mg, 0.2 mmol) under N₂. The reaction was heated to 100° C. and allowed to stir at this temperature for about 15 hours. The reaction mixture was then cooled to room temperature and filtered and the filtrate was washed with H₂O, brine, dried over Na₂SO₄, filtered and concentrated in vacuo. The residue obtained was purified using prep-TLC (petroleum ether:EtOAc=2:1) to provide 5-(3-(4-fluorobenzo[d]oxazol-2-yl)-4-nitrophenyl)-2-(4-fluorophenyl)-N-methyl-6-(N-methylmethylsulfonamido) benzofuran-3-carboxamide (51 mg, 82%). ¹H-NMR (CDCl₃, 400 MHz) δ 8.18 (s, 1H), 8.05 (d, J=2.4 Hz, 1H), 7.80˜7.88 (m, 3H), 7.58 (s, 1H), 7.28˜7.51 (m, 2H), 7.14 (t, J=8.4 Hz, 2H), 7.06 (t, J=8.8 Hz, 1H), 5.80 (d, J=4.0 Hz, 1H), 3.11 (s, 3H), 2.94 (d, J=4.8 Hz, 3H), 2.89 (s, 3H).

### Step 2—Synthesis of 5-(4-amino-3-(4-fluorobenzo[d]oxazol-2-yl)phenyl)-2-(4-fluorophenyl)-N-methyl-6-(N-methylmethylsulfonamido)benzofuran-3-carboxamide

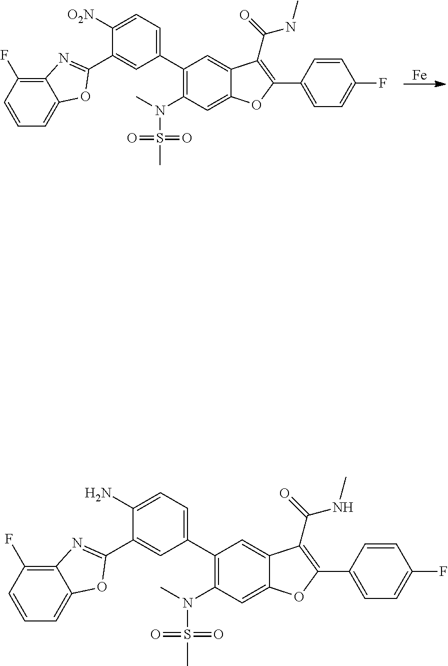

To a degassed solution of 5-(3-(4-fluorobenzo[d]oxazol-2-yl)-4-nitrophenyl)-2-(4-fluorophenyl)-N-methyl-6-(N-methylmethylsulfonamido)benzofuran-3-carboxamide (350 mg, 1.3 mmol) in MeOH (9 mL) was added Fe powder (270 mg, 5 mmol) and NH₄Cl (395 mg, 7.5 mmol) under N₂. The reaction was heated to 70° C. and allowed to stir at this temperature for about 15 hours, then was filtered and the filtrate was concentrated in vacuo to provide 5-(4-amino-3-(4-fluorobenzo[d]oxazol-2-yl)phenyl)-2-(4-fluorophenyl)-N-methyl-6-(N-methylmethylsulfonamido)benzofuran-3-carboxamide (300 mg, 94%). ¹H-NMR (CDCl₃, 400 MHz) δ 8.11 (s, 1H), 8.05 (d, J=2.4 Hz, 2H), 7.87˜7.91 (m, 2H), 7.78 (s, 1H), 7.36˜7.55 (m, 1H), 7.30 (d, J=7.2 Hz, 1H), 7.14 (t, J=8.8 Hz, 3H), 7.09 (t, J=8.8 Hz, 1H), 6.82 (d, J=8.4 Hz, 1H), 5.80 (d, J=4.4 Hz, 1H), 3.12 (s, 3H), 2.93 (d, J=4.8 Hz, 3H), 2.68 (s, 3H). MS (Ms+H)⁺: 603.

### Step 3—Synthesis of 5-(3-(4-fluorobenzo[d]oxazol-2-yl)-4-(methylsulfonamido) phenyl)-2-(4-fluorophenyl)-N-methyl-6-(N-methylmethylsulfonamido)benzofuran-3-carboxamide

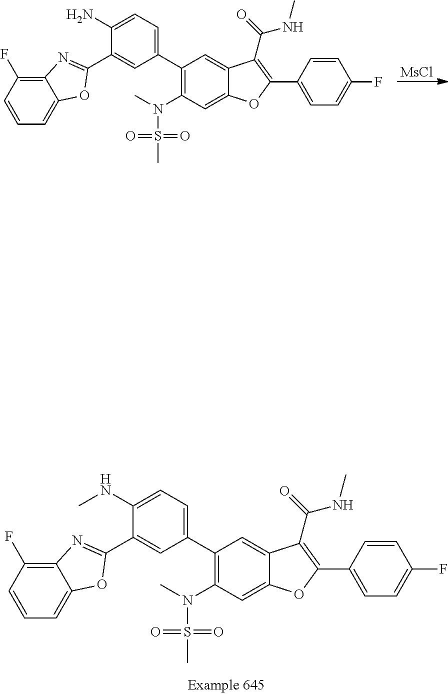

To a degassed solution of 5-(4-amino-3-(4-fluorobenzo[d]oxazol-2-yl)phenyl)-2-(4-fluorophenyl)-N-methyl-6-(N-methylmethylsulfonamido)benzofuran-3-carboxamide (40 mg, 0.07 mmol) and pyridine (1 mL) in dichloromethane (1 mL) was added MsCl (23 mg, 0.2 mmol) at 0° C. under N₂ atmosphere. The reaction was allowed stir for 5 hours, then the mixture was concentrated in vacuo and extracted with dichloromethane. The organic phase was washed with brine, dried over Na₂SO₄, filtered and concentrated in vacuo and the residue obtained was purified using prep-HPLC to provide the title compound (30 mg, 67%). ¹H-NMR (CDCl₃, 400 MHz) δ 8.30 (s, 1H), 7.85˜7.89 (m, 4H), 7.61 (t, J=6.4 Hz, 1H), 7.58 (d, J=5.6 Hz, 1H), 7.29˜7.36 (m, 2H), 7.16 (t, J=8.4 Hz, 2H), 7.07 (t, J=8.4 Hz, 1H), 5.77 (d, J=4.0 Hz, 1H), 3.13 (s, 3H), 3.12 (s, 3H), 2.93 (d, J=4.8 Hz, 3H), 2.75 (s, 3H). MS (Ms+H)⁺: 681.

### Example 646

### 5-(3-(4-fluorobenzo[d]oxazol-2-yl)-5-(1-hydroxyethyl)phenyl)-2-(4-fluorophenyl)-N-methyl-6-(N-methylmethylsulfonamido)benzofuran-3-carboxamide

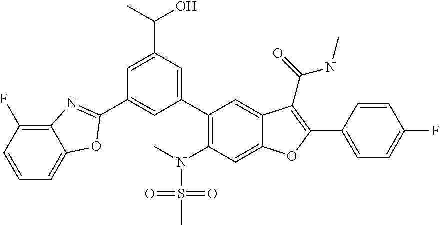

### Step 1—Synthesis of 5-(3-(4-fluorobenzo[d]oxazol-2-yl)-5-formylphenyl)-2-(4-fluorophenyl)-N-methyl-6-(N-methylmethylsulfonamido) benzofuran-3-carboxamide

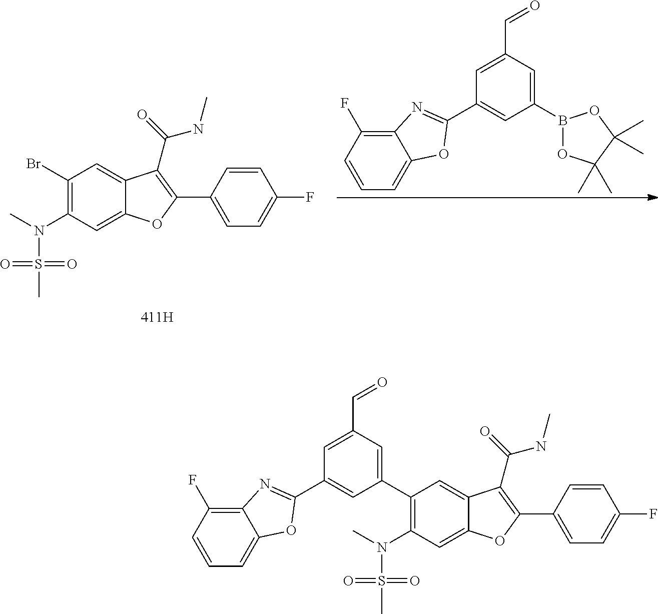

To a solution of 3-(4-fluorobenzo[d]oxazol-2-yl)-5-(4,4,5,5-tetramethyl-1,3,2-dioxaborolan-2-yl)benzaldehyde (40 mg, 0.11 mmol), Compound 411H (50 mg, 0.10 mmol) and K₃PO₄ (38 mg, 0.20 mmol) in DMF (2 mL) was added Pd(dppf)Cl₂ (10 mg) under N₂, and then the mixture was heated to 100° C. and allowed to stir at this temperature for 5 hours. The reaction mixture was cooled to room temperature and filtered and, the filtrate was concentrated in vacuo. The residue obtained was purified using prep-TLC (petroleum ether:EtOAc=5:1) to provide 5-(3-(4-fluorobenzo[d]oxazol-2-yl)-5-formylphenyl)-2-(4-fluorophenyl)-N-methyl-6-(N-methylmethylsulfonamido)benzofuran-3-carboxamide (35 mg, 53%). ¹H-NMR (CDCl₃, 400 MHz) 9.86 (s, 1H), 8.24 (s, 1H), 8.18 (s, 1H), 7.87˜7.90 (m, 2H), 7.84 (s, 1H), 7.74 (s, 1H), 7.54 (s, 1H), 7.34˜7.36 (m, 1H), 7.23˜7.29 (m, 1H), 7.12˜7.17 (m, 2H), 7.01˜7.06 (m, 1H), 5.89 (t, J=3.2 Hz, 1H), 3.03 (s, 3H), 2.94 (d, J=4.8 Hz, 3H), 2.75 (s, 3H). MS (M+H)⁺: 616.

### Step 2—Synthesis of 5-(3-(4-fluorobenzo[d]oxazol-2-yl)-5-(1-hydroxyethyl)phenyl)-2-(4-fluorophenyl)-N-methyl-6-(N-methylmethylsulfonamido)benzofuran-3-carboxamide

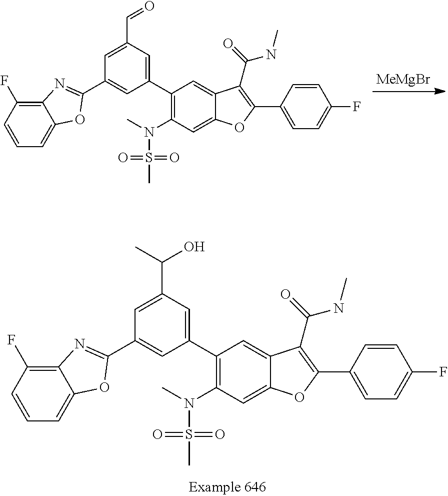

To a solution of 5-(3-(4-fluorobenzo[d]oxazol-2-yl)-5-formylphenyl)-2-(4-fluorophenyl)-N-methyl-6-(N-methylmethylsulfonamido)benzofuran-3-carboxamide (123 mg, 0.2 mmol) in anhydrous THF (5 mL) at 0° C. was added dropwise a solution of methylmagnesium bromide (0.67 mL, 3 N in ether). The reaction was allowed to stir at room temperature for 3 hours, and then the reaction mixture was quenched with saturated NH₄Cl, and extracted with CH₂Cl₂. The organic phase was dried (Na₂SO₄), filtered and concentrated in vacuo and the residue obtained was purified using column chromatography eluted with petroleum ether:EtOAc=3:1 to provide the title compound (100 mg, 70%). ¹H-NMR (CDCl₃, 400 MHz) 8.24 (s, 1H), 8.18 (s, 1H), 7.87˜7.90 (m, 2H), 7.84 (s, 1H), 7.74 (s, 1H), 7.54 (s, 1H), 7.34˜7.36 (m, 1H), 7.23˜7.29 (m, 1H), 7.12˜7.17 (m, 2H), 7.01˜7.06 (m, 1H), 5.89 (t, J=3.2 Hz, 1H), 5.00˜5.05 (m, 1H), 3.03 (s, 3H), 2.94 (d, J=4.8 Hz, 3H), 2.75 (s, 3H), 1.55 (d, J=6.4 Hz, 3H). MS (M+H)⁺: 632.

The following compound of the present invention was made using the method described above and using the appropriate reactants and/or reagents.

| | | | MS |
|---|---|---|---|
| Example | Structure | NMR | (M + H)⁺ |
| 647 | 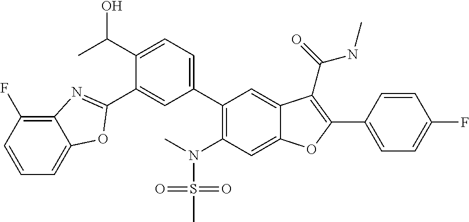 | ¹H-NMR (CDCl₃, 400 MHz) δ 8.22 (s, 1H), 7.92~7.97 (m, 3H), 7.83 (s, 1H), 7.73~7.76 (m, 2H), 7.49~7.71 (m, 1H), 7.37~7.42 (m, 1H), 7.22~7.27 (m, 2H), 7.12~7.17 (m, 1H), 5.93 (d, J = 4.0 Hz, 1H), 3.19 (s, 3H), 2.91 (s, 3H), 2.85 (s, 3H), 1.58 (d, J = 4.8 Hz, 3H). | 632 |

### Example 648

### 5-(3(4-fluorobenzo[d]oxazol-2-yl)-5-(1-fluoroethyl)phenyl)-2-(4-fluorophenyl)-N-methyl-6-(N-methylmethylsulfonamido)benzofuran-3-carboxamide

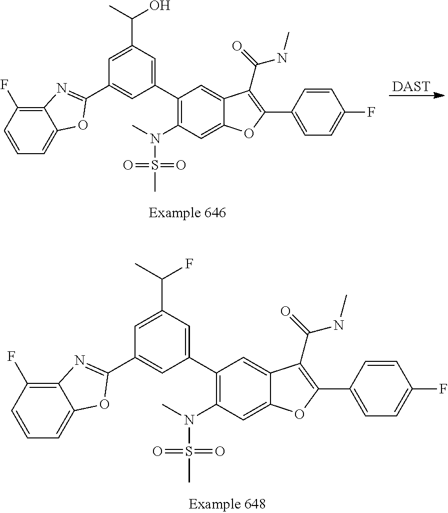

To a solution of the compound of Example 646 (73 mg, 0.12 mmol) in anhydrous CH₂Cl₂ (3 mL) at 0° C., was added DAST reagent (0.5 mL, 0.25 mmol) dropwise. The reaction was allowed to stir for 5 hours at room temperature, then the reaction was quenched with water, and extracted with CH₂Cl₂. The organic phase was washed with brine, dried over Na₂SO₄, filtered and concentrated in vacuo. The residue obtained was purified using column chromatography eluted with petroleum ether:EtOAc=3:1 to provide the title compound (35 mg, 50%). ¹H-NMR (CDCl₃, 400 MHz) 8.25 (s, 2H), 7.87˜7.91 (m, 2H), 7.84 (s, 1H), 7.65 (s, 1H), 7.58 (s, 1H), 7.29˜7.35 (m, 1H), 7.23˜7.28 (m, 1H), 7.13˜7.17 (m, 2H), 7.02˜7.06 (m, 1H), 5.70˜5.84 (m, 1H), 5.65˜5.76 (m, 1H), 3.09 (s, 3H), 2.94 (d, J=4.8 Hz, 3H), 2.69 (s, 3H), 1.69 (dd, J=6.4 Hz, 3H). MS (M+H)⁺: 634.

The following compound of the present invention was made using the method described above and using the appropriate reactants and/or reagents.

| | | | MS |
|---|---|---|---|
| Example | Structure | NMR | (M + H)⁺ |
| 649 | 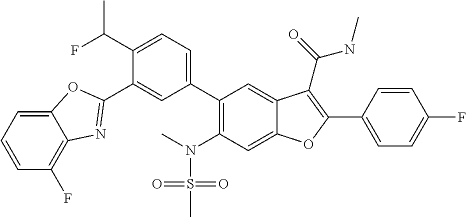 | ¹H-NMR (CDCl₃, 400 MHz) δ 7.87 (s, 1H), 7.80~7.87 (m, 4H), 7.64~7.67 (m, 1H), 7.58 (s, 1H), 7.32~7.34 (m, 1H), 7.24~7.29 (m, 1H), 7.12~7.19 (m, 2H), 7.01~7.05 (m, 1H), 6.77~6.91 (m, 1H), 5.84 (d, J = 8.0 Hz, 1H), 3.12 (s, 3H), 2.91 (d, J = 4.0 Hz, 3H), 2.65 (s, 3H), 1.73 (dd, J = 4.0 Hz, 3H). | 634 |

### Example 650

### 3-(4-fluorobenzo[d]oxazol-2-yl)-5-(2-(4-fluorophenyl)-3-(methylcarbamoyl)-6-(N-methylmethylsulfonamido)benzofuran-5-yl)benzoic acid

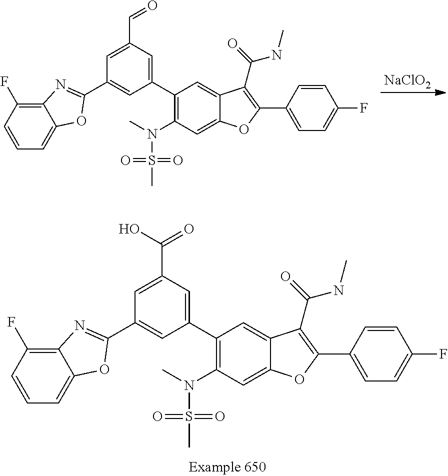

To a solution of 5-(3-(4-fluorobenzo[d]oxazol-2-yl)-5-formylphenyl)-2-(4-fluorophenyl)-N-methyl-6-(N-methylmethylsulfonamido)benzofuran-3-carboxamide (360 mg, 0.6 mmol, described in Example 423, step 1) in i-BuOH (2 mL) was added 2-methyl-2-butane (0.6 mL) and dioxane (2 mL) and the mixture was cooled to 0° C. To the cooled mixture was added as solution of NaClO₂ (600 mg, 6.6 mmol) and NaH₂PO₄ (1.2 g, 10.8 mmol) in water (3 mL) and the resulting reaction was allowed to stir at room temperature for 2 hours. CH₂Cl₂ was added and the organic phase was separated and washed with water, dried over Na₂SO₄, filtered and concentrated in vacuo. The residue obtained was purified using prep-HPLC to provide the title compound (35 mg, 53%). ¹H-NMR (CDCl₃, 400 MHz) 8.01˜8.89 (m, 2H), 7.61˜7.98 (m, 2H), 7.44˜7.46 (m, 1H), 7.35˜7.39 (m, 1H), 7.11˜7.19 (m, 4H), 6.94˜6.99 (m, 2H), 5.90 (s, 1H), 3.08 (s, 6H), 2.30 (s, 3H). MS (M+H)⁺: 632.

The following compound of the present invention was made using the method described above and using the appropriate reactants and/or reagents.

| | | | MS |
|---|---|---|---|
| Example | Structure | NMR | (M + H)⁺ |
| 651 | 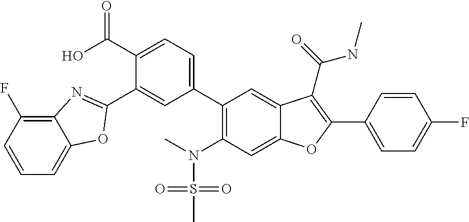 | H-NMR (CDCl₃, 400 MHz) δ 8.28~8.30 (m, 2H), 7.90~7.94 (m, 3H), 7.77 (d, J = 8.0 Hz, 1H), 7.63 (s, 1H), 7.33~7.41 (m, 2H), 7.18~7.22 (m, 2H), 7.09~7.14 (m, 1H), 5.84 (d, J = 8.0 Hz, 1H), 3.16 (s, 3H), 2.99 (d, J = 8.0 Hz, 3H), 2.82 (s, 3H). | 632 |

### Example 652

### 5-(3-carbamoyl-5-(4-fluorobenzo[d]oxazol-2-yl)phenyl)-2-(4-fluorophenyl)-N-methyl-6-(N-methylmethylsulfonamido)benzofuran-3-carboxamide

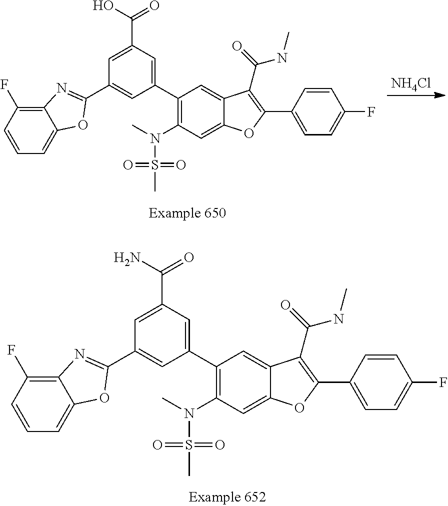

A solution of the compound of Example 650 (160 mg, 0.25 mmol), EDCI (67 mg, 0.25 mmol) and HOBT (96 mg, 0.25 mmol) in DMF (3 mL) was allowed to stir at room temperature for 3 hours. Et₃N (0.6 mL) and the NH₄Cl (20 mg, 0.4 mmol) were then added and the reaction was allowed to stir at room temperature for another 4 hours. The reaction mixture was concentrated in vacuo and the residue obtained was purified using prep-HPLC to provide the title compound (50 mg, 30%). ¹H-NMR (CDCl₃, 400 MHz) 8.79 (s, 1H), 8.52 (s, 1H), 8.26 (s, 1H), 7.93˜7.96 (m, 3H), 7.56 (s, 1H), 7.44˜7.46 (m, 1H), 7.35˜7.39 (m, 1H), 7.11˜7.19 (m, 3H), 6.94˜6.99 (m, 1H), 6.26 (s, 1H), 5.90 (s, 1H), 3.08 (s, 6H), 2.30 (s, 3H). MS (M+H)⁺: 631.

The following compounds of the present invention were made using the method described above and using the appropriate reactants and/or reagents.

| | | | MS |
|---|---|---|---|
| Example | Structure | NMR | (M + H)⁺ |
| 653 | 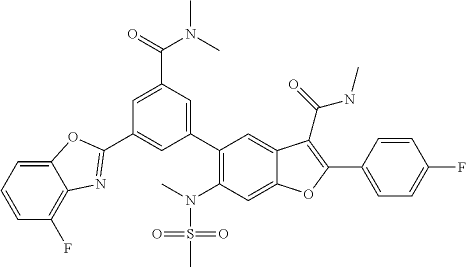 | ¹H-NMR (CDCl₃, 400 MHz) 8.22~8.23 (m, 2H), 7.67~7.69 (m, 3H), 7.64~7.67 (m, 1H), 7.56~7.59 (m, 1H), 7.33~7.35 (m, 1H), 7.26~7.29 (m, 1H), 7.11~7.14 (m, 2H), 7.04~7.06 (m, 1H), 6.02 (t, J = 0.8 Hz, 1H), 3.13-3.16 (m, 6H), 3.10 (s, 3H), 3.03 (m, 3H), 2.79 (t, J = 1.2 Hz, 3H). | 659 |
| 654 | 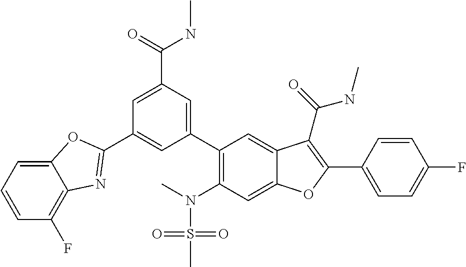 | ¹H-NMR (CDCl₃, 400 MHz) 8.88 (s, 1H), 8.51 (s, 1H), 8.26 (s, 1H), 7.85~7.90 (m, 3H), 7.61 (s, 1H), 7.36~7.39 (m, 1H), 7.26~7.31 (m, 1H), 7.13-7.17 (m, 2H), 7.03~7.07 (m, 1H), 5.89 (t, J = 0.8 Hz, 1H), 3.93 (s, 3H), 3.15 (s, 3H), 2.94 (d, J = 4.2 Hz, 3H), 2.70 (s, 3H). | 645 |
| 655 | 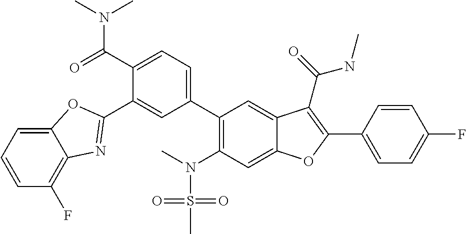 | ¹H-NMR (CDCl₃, 400 MHz) δ 8.33 (s, 1H), 7.90~7.92 (m, 2H), 7.88 (s, 1H), 7.67~7.84 (m, 1H), 7.61 (s, 1H), 7.43~7.45 (m, 1H), 7.27~7.34 (m, 2H), 7.15~7.24 (m, 2H), 7.02~7.07 (m, 1H), 6.11 (br s, 1H), 3.20 (s, 3H), 3.14 (s, 3H), 2.95 (d, J = 8.0 Hz, 3H), 2.85 (s, 3H), 2.80 (s, 3H). | 659 |
| 656 | 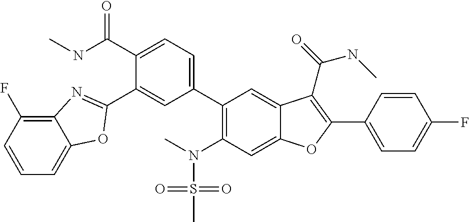 | ¹H-NMR (CDCl₃, 400 MHz) δ 8.15 (s, 1H), 7.85~8.15 (m, 2H), 7.77 (s, 1H), 7.60~7.69 (m, 2H), 7.53 (s, 1H), 7.30~7.32 (m, 1H), 7.25~7.28 (m, 1H), 7.12~7.16 (m, 2H), 6.99~7.04 (m, 1H), 6.46 (d, J = 4.0 Hz, 1H), 5.98 (d, J = 4.0 Hz, 1H), 3.07 (s, 3H), 2.98 (d, J = 4.0 Hz, 3H), 2.91 (d, J = 4.0 Hz, 3H), 2.78 (s, 3H). | 645 |
| 657 | 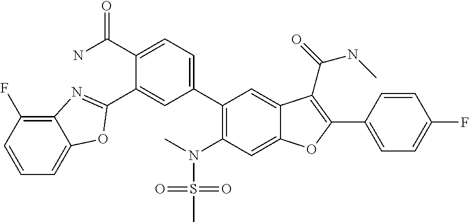 | ¹H-NMR (CDCl₃, 400 MHz) δ 8.16 (s, 1H), 7.88~7.91 (m, 2H), 7.79 (s, 1H), 7.63~7.70 (m, 2H), 7.55 (s, 1H), 7.34~7.36 (m, 1H), 7.24~7.32 (m, 1H), 7.15~7.19 (m, 2H), 7.03~7.07 (m, 1H), 6.83 (br s, 1H), 6.22 (d, J = 4.0 Hz, 1H), 6.02 (br s, 1H), 3.11 (s, 3H), 2.93 (d, J = 8.0 Hz, 3H), 2.82 (s, 3H). | 631 |

### 2-(4-fluorophenyl)-5-(4-methoxy-3-(6-(pyrimidin-5-yl)benzo[d]oxazol-2-yl)phenyl)-N-methyl-6-(N-methylmethylsulfonamido) benzofuran-3-carboxamide

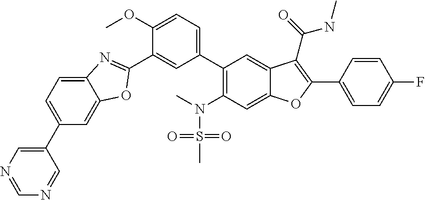

### Step 1—Synthesis of 2-(4-fluorophenyl)-5-(4-methoxy-3-(6-nitrobenzo[d]oxazol-2-yl)phenyl)-N-methyl-6-(N-methylmethylsulfonamido)benzofuran-3-carboxamide

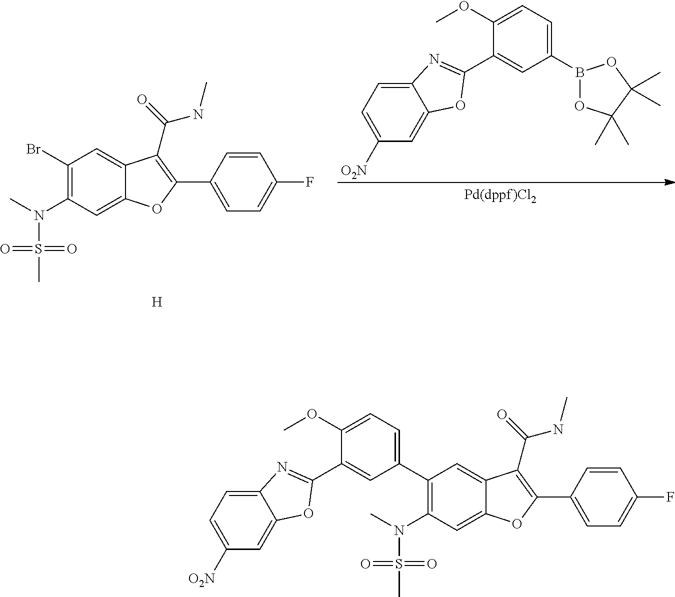

Compound 411H was converted to 2-(4-fluorophenyl)-5-(4-methoxy-3-(6-nitrobenzo[d]oxazol-2-yl)phenyl)-N-methyl-6-(N-methylmethylsulfonamido)benzofuran-3-carboxamide (810 mg, 16%) using the method described in Example 411, Step 12. ¹H-NMR (CDCl₃, 400 MHz) δ 8.43 (d, J=2.0 Hz, 1H), 8.26 (d, J=2.0 Hz, 1H), 8.21˜8.25 (m, 1H), 7.80˜7.88 (m, 4H), 7.67˜7.70 (m, 1H), 7.55 (s, 1H), 7.12˜7.17 (m, 3H), 5.81 (d, J=4.0 Hz, 1H), 4.04 (s, 3H), 3.11 (s, 3H), 2.93 (d, J=4.0 Hz, 3H), 2.76 (s, 3H).

### Step 2—Synthesis of 5-(3-(6-aminobenzo[d]oxazol-2-yl)-4-methoxyphenyl)-2-(4-fluorophenyl)-N-methyl-6-(N-methylmethylsulfonamido)benzofuran-3-carboxamide

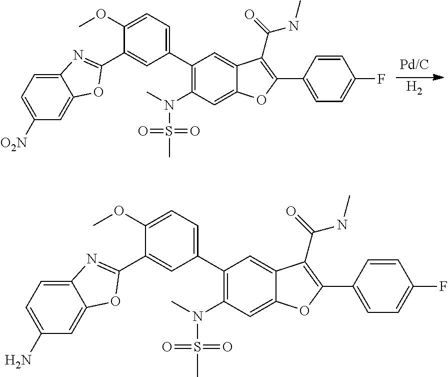

To a solution of 2-(4-fluorophenyl)-5-(4-methoxy-3-(6-nitrobenzo[d]oxazol-2-yl)phenyl)-N-methyl-6-(N-methylmethylsulfonamido)benzofuran-3-carboxamide (400 mg, 0.62 mmol) in MeOH (20 mL) was added Pd—C (10 mg) and the resulting reaction was stirred under 40 psi of H₂ atmosphere for 24 hours at room temperature. The reaction mixture was filtered and concentrated in vacuo to provide 5-(3-(6-aminobenzo[d]oxazol-2-yl)-4-methoxyphenyl)-2-(4-fluorophenyl)-N-methyl-6-(N-methylmethylsulfonamido)benzofuran-3-carboxamide (350 mg, 92%), which was used without further purification. MS (M+H)⁺: 615.

### Step 3—Synthesis of 2-(4-fluorophenyl)-5-(3-(6-iodobenzo[d]oxazol-2-yl)-4-methoxyphenyl)-N-methyl-6-(N-methylmethylsulfonamido)benzofuran-3-carboxamide

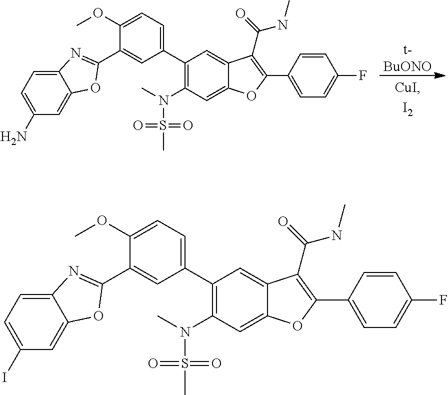

To a 0° C. suspension of 5-(3-(6-aminobenzo[d]oxazol-2-yl)-4-methoxyphenyl)-2-(4-fluorophenyl)-N-methyl-6-(N-methylmethylsulfonamido)benzofuran-3-carboxamide (620 mg, 1.01 mmol), I₂ (200 mg, 0.81 mol), CuI (190 mg, 1.01 mmol) in THF was added t-BuONO dropwise. The reaction was allowed to stir at 0° C. for 1 hour and then stirred at refluxed for about 15 hours. The reaction was then cooled to room temperature, diluted with dichloromethane and filtered. The filtrate was concentrated in vacuo and the residue obtained was purified using column chromatography eluted with petroleum ether:EtOAc=3:1 to provide 2-(4-fluorophenyl)-5-(3-(6-iodobenzo[d]oxazol-2-yl)-4-methoxyphenyl)-N-methyl-6-(N-methylmethylsulfonamido)benzofuran-3-carboxamide (360 mg, 53.8%) as a yellow solid. MS (M+H)⁺: 726.

### Step 4—Synthesis of 2-(4-fluorophenyl)-5-(4-methoxy-3-(6-(pyrimidin-5-yl)benzo[d]oxazol-2-yl)phenyl)-N-methyl-6-(N-methylmethylsulfonamido)benzofuran-3-carboxamide

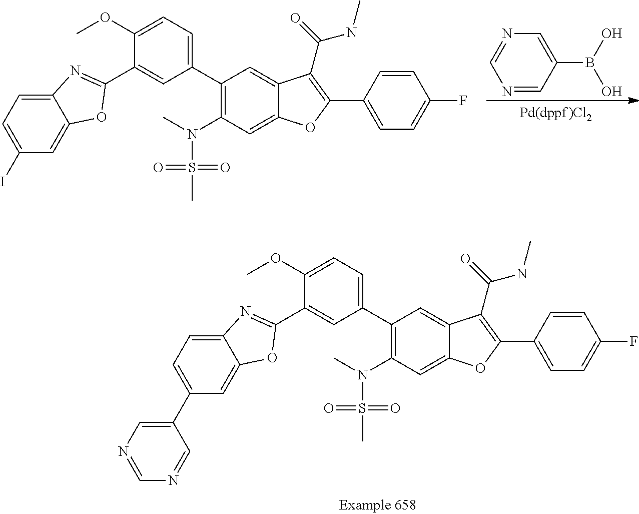

To a degassed solution of 2-(4-fluorophenyl)-5-(3-(6-iodobenzo[d]oxazol-2-yl)-4-methoxyphenyl)-N-methyl-6-(N-methylmethylsulfonamido)benzofuran-3-carboxamide (100 mg, 0.14 mmol), pyrimidin-5-ylboronic acid (26 mg, 0.21 mmol) and K₃PO₄ (75 mg, 0.28 mmol) in dry DMF (3 mL) was added Pd(dppf)Cl₂ (3 mg) under N₂. The reaction was heated to 100° C. and allowed to stir at this temperature for 6 hours. The reaction mixture was cooled to room temperature and filtered, and the filtrate was washed with H₂O, brine, dried over Na₂SO₄, filtered and concentrated in vacuo. The residue obtained was purified using prep-HPLC to provide the title compound (45 mg, 48.3%). ¹H-NMR (CDCl₃, 400 MHz) δ 9.29 (s, 1H), 9.13 (s, 2H), 8.30 (s, 1H), 8.01 (d, J=8.0 Hz, 1H), 7.95˜7.98 (m, 2H), 7.93 (s, 1H), 7.87 (s, 1H), 7.76 (d, J=6.4 Hz, 1H), 7.64 (d, J=11.2 Hz, 2H), 7.23˜7.27 (m, 3H), 5.96 (s, 1H), 4.14 (s, 3H), 3.21 (s, 3H), 3.03 (d, J=4.8 Hz, 3H), 2.85 (s, 3H). MS (M+H)⁺: 678.

The following compounds of the present invention were made using the method described above and using the appropriate reactants and/or reagents.

| | | | MS |
|---|---|---|---|
| Example | Structure | NMR | (M + H)⁺ |
| 659 | 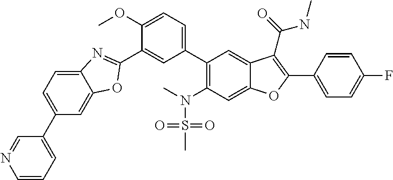 | ¹H-NMR (CDCl₃, 400 MHz) δ 8.44 (s, 1H), 8.26 (d, J = 8.0 Hz, 1H), 8.19 (d, J = 2.0 Hz, 1H), 7.85~7.89 (m, 3H), 7.85 (s, 1H), 7.64~7.67 (m, 1H), 7.56 (s, 1H), 7.35 (d, J = 8.0 Hz, 1H), 7.23~7.28 (m, 1H), 7.12~7.16 (m, 3H), 7.03 (t, J = 8.0 Hz, 1H), 5.96 (d, J = 4.0 Hz, 1H), 4.03 (s, 3H), 3.12 (s, 3H), 2.95 (d, J = 4.8 Hz, 3H), 2.75 (s, 3H). | 677 |
| 660 | 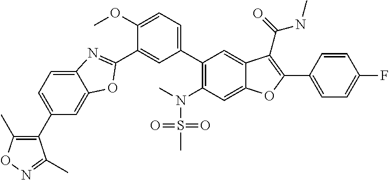 | ¹H-NMR (CDCl₃, 400 MHz) δ 8.24 (d, J = 1.6 Hz, 1H), 7.87~7.94 (m, 4H), 7.73~7.76 (m, 1H), 7.62 (s, 1H), 7.51 (s, 1H), 7.29 (d, J = 8.0 Hz, 1H), 7.22 (t, J = 8.0 Hz, 3H), 6.03 (s, 1H), 4.08 (s, 3H), 3.16 (s, 3H), 3.01 (d, J = 4.0 Hz, 3H), 2.83 (s, 3H), 2.45 (s, 3H), 2.32 (s, 3H). | 695 |
| 661 | 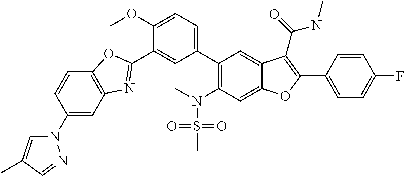 | ¹H-NMR (CDCl₃, 400 MHz) δ 8.16 (s, 1H), 7.87~7.91 (m, 3H), 7.81 (s, 1H), 7.61~7.70 (m, 3H), 7.55 (d, J = 8.4 Hz, 2H), 7.48 (s, 1H), 7.14 (t, J = 8.8 Hz, 3H), 5.81 (d, J = 4.4 Hz, 1H), 4.03 (s, 3H), 3.11 (s, 3H), 2.93 (d, J = 4.8 Hz, 3H), 2.72 (s, 3H), 2.14 (s, 3H). | 680 |
| 662 | 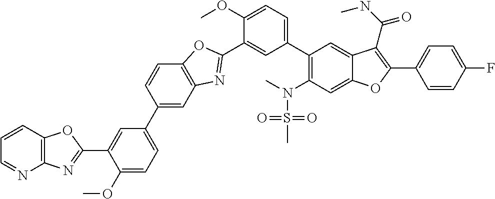 | ¹H-NMR (CDCl₃, 400 MHz) δ 8.60 (s, 1H), 8.53 (s, 1H), 8.25 (d, J = 3.0 Hz, 1H), 8.05 (s, 1H), 7.97 (t, J = 3.2 Hz, 2H), 7.89 (s, 1H), 7.82 (d, J = 8.4 Hz, 1H), 7.69 (d, J = 8.4 Hz, 1H), 7.65 (d, J = 8.4 Hz, 2H), 7.32 (d, J = 4.8 Hz, 1H), 7.30 (d, J = 4.8 Hz, 1H), 7.19~7.23 (m, 4H), 5.93 (t, J = 4.8 Hz, 1H), 4.12 (s, 3H), 4.09 (s, 3H), 3.19 (s, 3H), 3.02 (d, J = 5.2 Hz, 3H), 2.79 (s, 3H). | 824 |
| 663 | 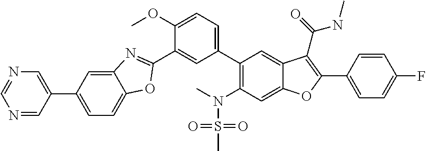 | ¹H-NMR (CDCl₃, 400 MHz) δ 9.16 (s, 1H), 8.98 (s, 2H), 8.20 (s, 1H), 7.97 (s, 1H), 7.83~7.90 (m, 2H), 7.78 (s, 1H), 7.65~7.69 (m, 2H), 7.50~7.56 (m, 2H), 7.16 (t, J = 7.6 Hz, 3H), 5.81 (br s, 1H), 4.05 (s, 3H), 3.12 (s, 3H), 2.93 (d, J = 3.2 Hz, 3H), 2.74 (s, 3H). | 792 |
| 664 | 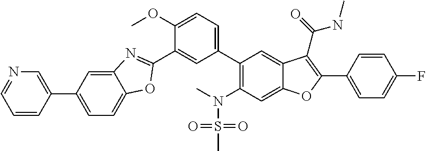 | ¹H-NMR (CDCl₃, 400 MHz) δ 8.80 (d, J = 5.2 Hz, 1H), 8.55 (d, J = 7.6 Hz, 1H), 8.18 (s, 1H), 8.10 (s, 1H), 7.81~7.95 (m, 5H), 7.55~7.73 (m, 4H), 7.13~7.19 (m, 3H), 6.04 (d, J = 4.4 Hz, 1H), 4.02 (s, 3H), 3.11 (s, 3H), 2.94 (d, J = 3.6 Hz, 3H), 2.79 (s, 3H). | 791 |
| 665 | 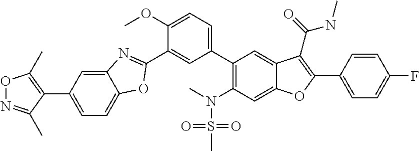 | ¹H-NMR (CDCl₃, 400 MHz) δ 8.16 (d, J = 2.0 Hz, 1H), 7.86~7.89 (m, 2H), 7.82 (s, 1H), 7.56~7.67 (m, 5H), 7.13~7.18 (m, 3H), 5.85 (s, 1H), 4.03 (s, 3H), 3.11 (s, 3H), 2.94 (d, J = 4.8 Hz, 3H), 2.74 (s, 3H), 2.37 (s, 3H), 2.24 (s, 3H). | 695 |

### Example 666

### 5-(3-(3-(3-((dimethylamino)methyl)-1H-indol-2-yl)phenyl)-2-(4-fluorophenyl)-N-methyl-6-(N-methylmethylsulfonamido)benzofuran-3-carboxamide

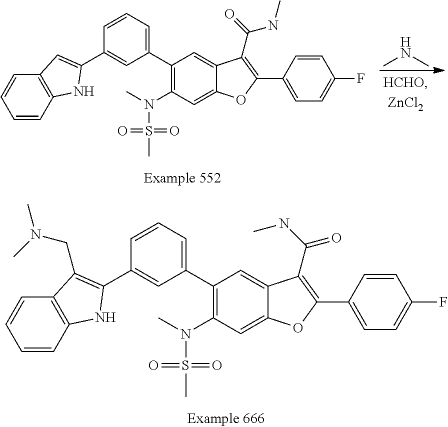

A solution of the compound of Example 552 (50 mg, 0.09 mmol), polyoxymethylene (3 mg, 0.09 mmol), dimethylamine (41 mg, 0.9 mmol), ZnCl₂ (41 mg, 0.27 mmol) in EtOH (2 mL) was heated to 60° C. and allowed to stir at this temperature for 12 hours. The reaction mixture was added to water and then extracted with ethyl acetate and the organic extract was washed with brine, dried over Na₂SO₄, filtered and concentrated in vacuo. The residue obtained was purified using prep-HPLC to provide Compound 256 (10 mg, 20%). ¹H-NMR (CDCl₃, 400 MHz) δ 9.63 (s, 1H), 7.89˜7.97 (m, 4H), 7.58˜7.67 (m, 3H), 7.47˜7.49 (m, 3H), 7.19˜7.30 (m, 4H), 6.11 (s, 1H), 4.69 (s, 2H), 3.16 (s, 3H), 2.95˜3.00 (m, 6H), 2.55 (s, 6H).

MS (M+H)⁺: 625.

### Example 667

### 5-(3-(3-((1H-imidazol-1-yl)methyl)-1H-indol-2-yl)phenyl)-2-(4-fluorophenyl)-N-methyl-6-N-methylmethylsulfonamido)benzofuran-3-carboxamide

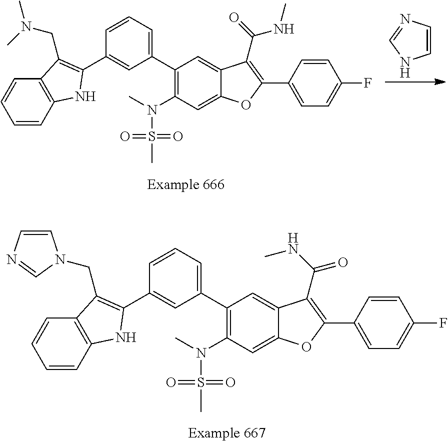

A solution of the compound of Example 666 (55 mg, 0.09 mmol) and imidazole (31 mg, 0.45 mmol) in xylenes (1.5 mL) was heated to 120° C. and allowed to stir at this temperature for 1 hour. The reaction mixture was cooled to room temperature and concentrated in vacuo and the residue obtained was purified using prep-HPLC to provide the title compound (30 mg, 60%). ¹H-NMR (CDCl₃, 400 MHz) δ 9.51 (s, 1H), 7.93˜7.96 (m, 2H), 7.77˜7.82 (m, 2H), 7.57 (s, 1H), 7.38˜7.49 (m, 5H), 7.12˜7.24 (m, 4H), 6.95˜6.98 (m, 2H), 6.32 (s, 1H), 5.39 (s, 2H), 3.04 (s, 3H), 2.96˜2.97 (m, 3H), 2.92 (s, 3H). MS (M+H)⁺: 648.

### Example 668

### 5-(3-(1-(2-aminoethyl)-1H-indol-2-yl)phenyl)-2-(4-fluorophenyl)-N-methyl-6-(N-methylmethylsulfonamido)benzofuran-3-carboxamide

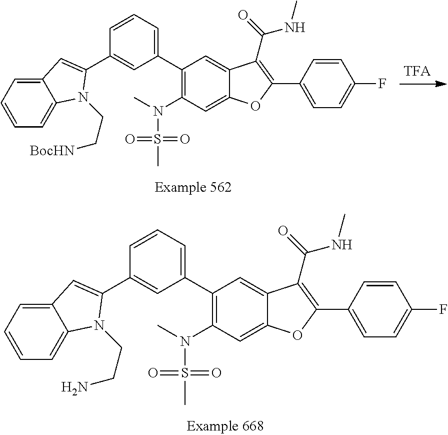

The compound of Example 562 (50 mg, 0.07 mmol) was added to a 0° C. mixture of TFA/dichloromethane (1:4, 1 mL). The reaction was allowed to stir at room temperature for 1.5 hours, then saturated aqueous NaHCO₃ was added to adjust the reaction mixture to pH 7. The reaction mixture was then extracted with EtOAc (30 mL) and the organic extract was washed with saturated aqueous NaHCO₃ (2×10 mL), brine (2×20 mL), dried over Na₂SO₄, filtered and concentrated in vacuo. The residue obtained was purified using prep-TLC (dichloromethane:MeOH=15:1) to provide the title compound (40 mg, 93%). ¹H-NMR (CDCl₃, 400 MHz) δ 7.92˜7.95 (m, 2H), 7.83 (s, 1H), 7.62˜7.64 (d, J=7.6 Hz, 1H), 7.56 (s, 2H), 7.51˜7.52 (d, J=5.6 Hz, 2H), 7.41˜7.44 (m, 2H), 7.12˜7.24 (m, 4H), 6.58 (s, 1H), 6.07˜6.08 (d, J=4.4 Hz, 1H), 4.30˜4.33 (t, d, J=6.8 Hz, 2H), 3.22 (s, 3H), 2.91˜2.96 (m, 5H), 2.69 (s, 3H).

MS (M+H)⁺: 611.

### Example 669

### 2-(4-fluorophenyl)-N-methyl-6-(N-methylmethylsulfonamido)-5-(3-(3-(pyridin-3-yl)-1H-indol-2-yl)phenyl)benzofuran-3-carboxamide

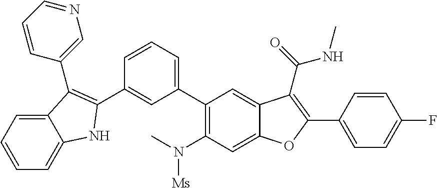

Step 1—Synthesis of 5-(3-(3-bromo-1H-indol-2-yl)phenyl)-2-(4-fluorophenyl)-N-methyl-6-(N-methylmethylsulfonamido)benzofuran-3-carboxamide

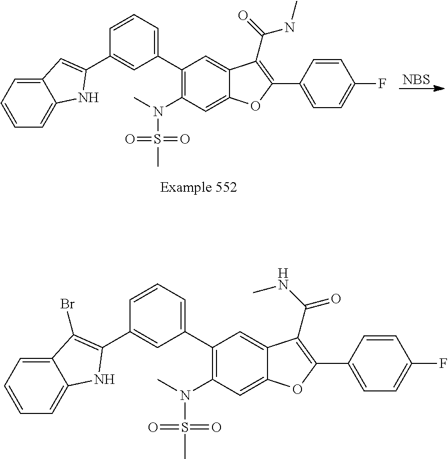

To a solution of the compound of example 552 (50 mg, 0.09 mmol) in 3 mL of DMF, was added NBS (16 mg, 0.09 mmol) and the resulting reaction mixture was placed under N₂ atmosphere, heated to 75° C. and allowed to stir at this temperature for 4 hours. The reaction mixture was then concentrated in vacuo and the residue obtained was diluted with EtOAc. The resulting solution was washed with brine, dried over Na₂SO₄, filtered and concentrated in vacuo and the residue obtained was purified using prep-TLC (petroleum ether:EtOAc=2:1) to provide 5-(3-(3-bromo-1H-indol-2-yl)phenyl)-2-(4-fluorophenyl)-N-methyl-6-(N-methylmethylsulfonamido)benzofuran-3-carboxamide (40 mg, 89%) as white solid. ¹H-NMR (CDCl₃, 400 MHz) δ 9.38 (s, 1H), 8.02 (d, J=8.0 Hz, 1H), 7.94 (s, 1H), 7.88˜7.94 (m, 2H), 7.84 (s, 1H), 7.53 (t, J=7.6 Hz, 2H), 7.46 (d, J=4.8 Hz, 1H), 7.35˜7.40 (m, 2H), 7.11˜7.15 (m, 4H), 5.80 (s, 1H), 3.04 (s, 3H), 2.94 (d, J=5.2 Hz, 3H), 2.87 (s, 3H). MS (M+H)⁺: 646/648.

### Step 2—Synthesis of 2-(4-fluorophenyl)-N-methyl-6-(N-methylmethylsulfonamido)-5-(3-(3-(pyridin-3-yl)-1H-indol-2-yl)phenyl)benzofuran-3-carboxamide

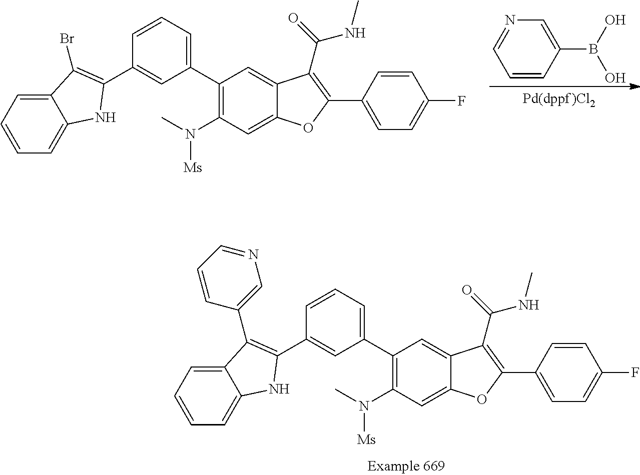

A mixture of 5-(3-(3-bromo-1H-indol-2-yl)phenyl)-2-(4-fluorophenyl)-N-methyl-6-(N-methylmethylsulfonamido)benzofuran-3-carboxamide (100 mg, 0.15 mmol), pyridin-3-ylboronic acid (24 mg, 0.19 mmol), Pd(dppf)Cl₂(12 mg) and K₃PO₄.3H₂O (82 mg, 0.31 mmol) in ethanol and water (2.5 mL, 4:1) was placed in a commercial microwave oven and subjected to microwave irradiation for 30 minutes (120 watts, internal reaction temperature was 100° C. at the conclusion of irradiation). The reaction mixture was cooled to room temperature and then was diluted with water and extracted with EtOAc. The organic extract was washed with brine, dried over Na₂SO₄, filtered and concentrated in vacuo and the residue obtained was purified using prep-HPLC to provide the title compound (10 mg, 10%). ¹H-NMR (MeOD, 400 MHz) δ 8.74 (s, 1H), 8.60 (d, J=6.8 Hz, 2H), 7.93˜8.00 (m, 3H), 7.84 (s, 1H), 7.72 (d, J=8.0 Hz, 1H), 7.59 (d, J=4.0 Hz, 2H), 7.54 (d, J=12.0 Hz, 4H), 7.26˜7.30 (m, 3H), 7.20 (t, J=4.0 Hz, 1H), 3.17 (s, 3H), 2.94 (d, J=4.0 Hz, 6H). MS (M+H)⁺: 645.

The following compound of the present invention was made using the method described above and using the appropriate reactants and/or reagents.

| | | | MS |
|---|---|---|---|
| Example | Structure | NMR | (M + H)⁺ |
| 670 | 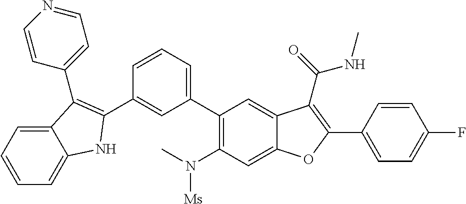 | ¹H-NMR (MeOD, 400 MHz) δ 8.43 (d, J = 4.0 Hz, 2H), 7.94~7.97 (m, 2H), 7.80 (s, 1H), 7.71 (d, J = 8.0 Hz, 1H), 7.63 (s, 2H), 7.46~7.50 (m, 6H), 7.21~7.28 (m, 3H), 7.12~7.16 (m, 1H), 3.10 (s, 3H), 2.92 (s, 3H), 2.94 (s, 3H). | 645 |

### Example 671

### 2-(2-(3-(2-(4-fluorophenyl)-3-(methylcarbamoyl)-6-(N-methylmethylsulfonamido)benzofuran-5-yl)phenyl)-1H-indol-1-yl)acetic acid

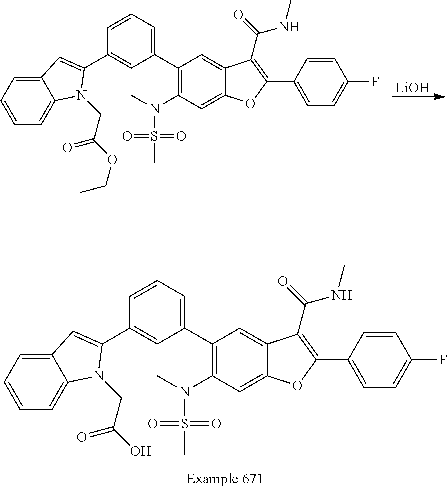

To the solution of ethyl 2-(2-(3-(2-(4-fluorophenyl)-3-(methylcarbamoyl)-6-(N-methylmethylsulfonamido)benzofuran-5-yl)phenyl)-1H-indol-1-yl)acetate (120 mg, 0.18 mmol, prepared according to the method described in Example 411) in MeOH (1.5 mL) was added a saturated solution of LiOH. The reaction was allowed to stir at room temperature until LCMS indicated that the starting material was consumed. The reaction mixture was extracted with dichloromethane and the organic extract was washed with brine, dried over Na₂SO₄, filtered and concentrated in vacuo. The residue obtained was purified using HPLC to provide the title compound (110 mg, 95.7%). ¹H-NMR (CDCl₃, 400 MHz) δ 7.81˜7.91 (m, 3H), 7.64 (d, J=7.6 Hz, 1H), 7.56 (s, 1H), 7.52 (t, J=6.4 Hz, 4H), 7.26˜7.15 (m, 5H), 6.63 (s, 1H), 6.19 (s, 1H), 4.88 (s, 2H), 3.12 (s, 3H), 2.94 (d, J=4.0 Hz, 3H), 2.70 (s, 3H). MS (M+H)⁺: 626.

The following compounds of the present invention were made using the method described above and using the appropriate reactants and/or reagents.

| | | | MS |
|---|---|---|---|
| Example | Structure | NMR | (M + H)⁺ |
| 672 | 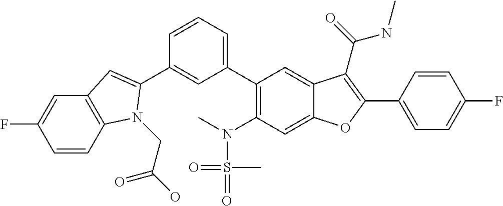 | ¹H-NMR (CDCl₃, 400 MHz) δ 7.79 (s, 2H), 7.64 (s, 1H), 7.43~7.49 (m, 4H), 7.15~7.30 (m, 3H), 6.87~6.99 (m, 2H), 6.47 (s, 1H), 6.12~6.17 (m, 1H), 4.60 (s, 2H), 2.92 (s, 3H), 2.75 (d, J = 2.4 Hz, 3H), 2.44 (s, 3H). | 644 |
| 673 | 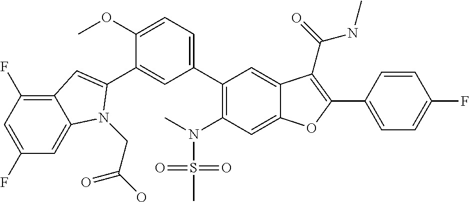 | ¹H-NMR (DMSO-d6, 400 MHz) δ 8.55 (d, J = 8.0 Hz, 1H), 7.99~8.03 (m, 2H), 7.97 (s, 1H), 7.62 (s, 1H), 7.56~7.59 (m, 1H), 7.47 (d, J = 2.0 Hz, 1H), 7.41 (t, J = 8.8 Hz, 2H), 7.20~7.26 (m, 2H), 6.87 (t, J = 8.0 Hz, 1H), 6.58 (s, 1H), 4.59 (s, 1H), 4.65 (s, 1H), 3.80 (s, 3H), 3.05 (s, 3H), 3.01 (s, 3H), 2.83 (d, J = 4.8 Hz, 3H). | 692 |

### Example 674

### 5-(3-(1-(2-amino-2-oxoethyl)-1H-indol-2-yl)phenyl)-2-(4-fluorophenyl)-N-methyl-6-(N-methylmethylsulfonamido)benzofuran-3-carboxamide

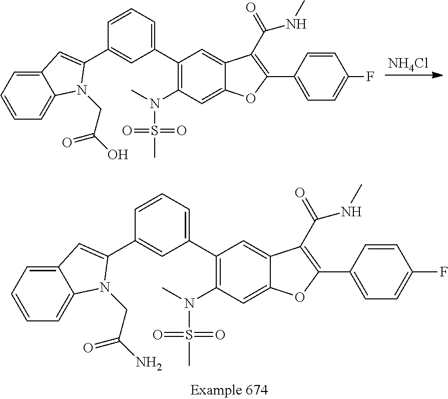

To a solution of the compound of Example 671 (50 mg, 0.08 mmol) in MeCN (1 mL) was added EDCI (23 mg, 0.12 mmol) and HOBT (16 mg, 0.12 mmol). The reaction was allowed to stir at room temperature for 2 hours then TEtOAc (16 mg, 0.16 mmol) and NH₄Cl (9 mg, 0.16 mmol) were added. The reaction was then stirred at room temperature and monitored using LCMS until the starting material was consumed completely. The reaction mixture was concentrated in vacuo and the residue obtained was purified using prep-HPLC to provide the title compound (10 mg, 20%). ¹H-NMR (CDCl₃, 400 MHz) δ 8.00 (t, J=8.0 Hz, 2H), 7.83 (s, 1H), 7.68 (d, J=8.0 Hz, 1H), 7.60˜7.52 (m, 1H), 7.36 (d, J=8:0 Hz, 4H), 7.31 (d, J=7.2 Hz, 2H), 7.20 (t, J=8.4 Hz, 3H), 6.75 (s, 1H), 6.22 (s, 1H), 5.74 (d, J=11.6 Hz, 1H), 5.57 (s, 1H), 4.83 (s, 2H), 3.19 (s, 3H), 3.03 (d, J=4.4 Hz, 3H), 2.72 (s, 3H). MS (M+H)⁺: 625.

The following compound of the present invention was made using the method described above and using the appropriate reactants and/or reagents.

| | | | MS |
|---|---|---|---|
| Example | Structure | NMR | (M + H)⁺ |
| 675 | 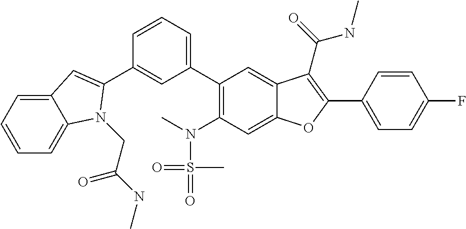 | ¹H-NMR (CDCl₃, 400 MHz) δ 8.03~8.00 (m, 2H), 7.81 (s, 1H), 7.69 (d, J = 8.0 Hz, 1H), 7.60 (s, 1H), 7.57~7.47 (m, 4H), 7.33~7.28 (m, 2H), 7.26~7.18 (m, 3H), 6.76 (s, 1H), 6.31 (d, J = 4.0 Hz, 1H), 5.77 (d, J = 4.4 Hz, 1H), 4.81 (s, 2H), 3.19(s, 3H), 3.05 (d, J = 4.8 Hz, 3H), 2.69 (t, J = 4.0 Hz, 6H). | 639 |

### Example 676

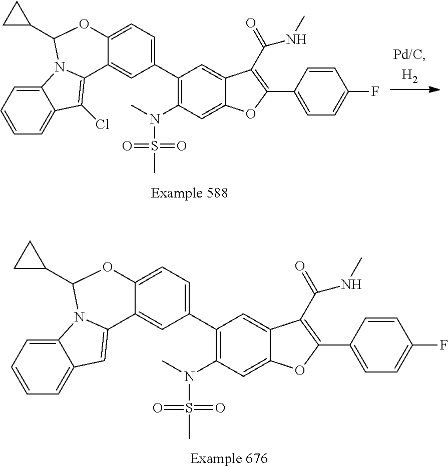

To a solution of the compound of Example 588 (50 mg, 0.08 mmol) in MeOH (1 mL) was added Pd/C (10 mg), and the mixture was put under H₂ atmosphere (50 psi) and allowed to stir for about 15 hours. The reaction mixture was filtered, the filtrate was concentrated in vacuo and the residue obtained was purified using prep-HPLC to provide the title compound (20 mg, 40%). ¹H-NMR (CDCl₃, 400 MHz) δ 7.95˜7.92 (m, 2H), 7.84 (s, 1H), 7.80 (d, J=2.0 Hz, 1H), 7.62 (d, J=10.8 Hz, 2H), 7.42 (d, J=8.0 Hz, 1H), 7.34˜7.31 (m, 1H), 7.24˜7.11 (m, 5H), 6.85 (s, 1H), 5.94 (d, J=4.8 Hz, 1H), 5.82 (d, J=7.6 Hz, 1H), 3.16 (s, 3H), 2.99 (d, J=5.2 Hz, 3H), 2.71 (s, 3H), 1.58˜1.50 (m, 1H), 0.67˜0.57 (m, 4H). MS (M+H)⁺: 636.

### Example 677

### 5-(3-ethynylphenyl)-2-(4-fluorophenyl)-N-methyl-6-(N-methylmethylsulfonamido)benzofuran-3-carboxamide

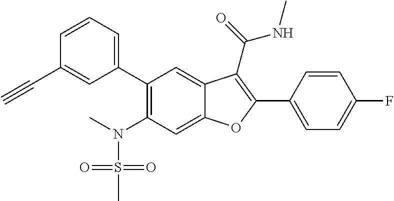

### Step 1—Synthesis of 2-(4-fluorophenyl)-N-methyl-1-(N-methylmethylsulfonamido)-543-((trimethylsilyl)ethynyl)phenyl)benzofuran-3-carboxamide

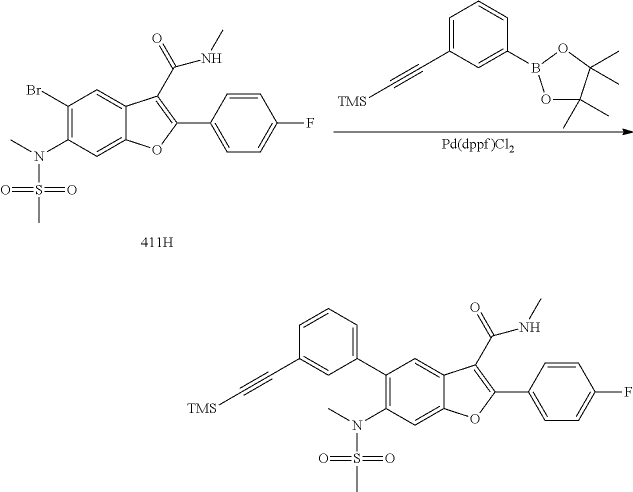

To a mixture of trimethyl((3-(4,4,5,5-tetramethyl-1,3,2-dioxaborolan-2-yl)phenyl)ethynyl)silane (480 mg, 1.60 mmol), Compound 411H (600 mg, 1.32 mmol) and K₃PO₄.3H₂O (700 mg, 1.99 mmol) in 1,4-dioxane (20 mL), was added Pd(dppf)Cl₂ (15 mg). The reaction was put under N₂ atmosphere, heated to 80° C. and allowed to stir at this temperature for 2 hours. The reaction mixture was then concentrated in vacuo and the residue obtained was diluted with water and extracted with EtOAc. The organic extract was washed with brine, dried (Na₂SO₄), filtered and concentrated in vacuo and the residue obtained was purified using column chromatography eluted with petroleum ether:EtOAc=3:1 to provide 2-(4-fluorophenyl)-N-methyl-6-(N-methylmethylsulfonamido)-5-(3-((trimethylsilyl)ethynyl)phenyl)benzofuran-3-carboxamide (600 mg, 83%). ¹H-NMR (CDCl₃, 400 MHz) δ 7.90˜7.94 (m, 2H), 7.75 (s, 1H), 7.59 (s, 1H), 7.47˜7.52 (m, 2H), 7.37˜7.43 (m, 2H), 7.16˜7.21 (m, 2H), 5.93 (br s, 1H), 3.13 (s, 3H), 2.98 (d, J=4.8 Hz, 3H), 2.62 (s, 3H) 0.26 (s, 9H). MS (M+H)⁺: 549.

### Step 2—Synthesis of 5-(3-ethynylphenyl)-2-(4-fluorophenyl)-N-methyl-6-(N-methylmethylsulfonamido)benzofuran-3-carboxamide

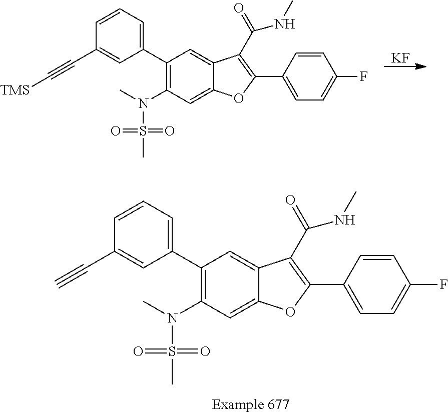

To a solution of 2-(4-fluorophenyl)-N-methyl-6-(N-methylmethylsulfonamido)-5-(3-((trimethylsilyl)ethynyl)phenyl)benzofuran-3-carboxamide (600 mg, 1.09 mmol) in MeOH was added KF (200 mg, 3.44 mmol) and the reaction was allowed to stir at room temperature for about 15 hours. The reaction mixture was concentrated in vacuo and the residue obtained was diluted with water and extracted with EtOAc. The organic extract was washed with brine, dried (Na₂SO₄), filtered and concentrated in vacuo and the residue obtained was purified using column chromatography eluted with petroleum ether:EtOAc=3:1 to provide the title compound (300 mg, 57%). ¹H-NMR (CDCl₃, 400 MHz) δ 7.91˜7.95 (m, 2H), 7.77 (s, 1H), 7.61 (s, 1H), 7.51˜7.56 (m, 2H), 7.39˜7.47 (m, 2H), 7.17˜7.23 (m, 2H), 5.87 (br s, 1H), 3.14 (s, 3H), 3.11 (s, 1H), 2.99 (d, J=4.8 Hz, 3H), 2.63 (s, 3H). MS (M+H)⁺: 477.

The following compound of the present invention was made using the method described above and using the appropriate reactants and/or reagents.

| | | | MS |
|---|---|---|---|
| Example | Structure | NMR | (M + H)⁺ |
| 678 | 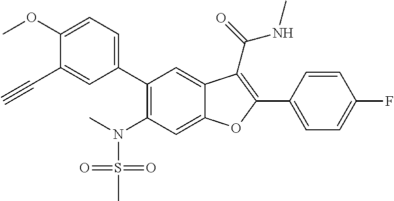 | ¹H-NMR (CDCl₃, 400 MHz) δ 7.90~7.93 (m, 2H), 7.74 (s, 1H), 7.57 (s, 1H), 7.51 (d, J = 2.0 Hz, 1H), 7.43~7.46 (m, 1H), 7.18 (t, J = 8.6 Hz, 2H), 6.96 (d, J = 8.4 Hz, 1H), 5.87 (d, J = 4.4 Hz, 1H), 3.95 (s, 3H), 3.32 (s, 1H), 3.31 (s, 3H), 2.98 (d, J = 4.8 Hz, 3H), 2.70 (s, 3H). | 507 |

### Example 679

### 5-(3-(5-bromofuro[2,3-b]pyridin-2-yl)phenyl)-2-(4-fluorophenyl)-N-methyl-6-(N-methylmethylsulfonamido)benzofuran-3-carboxamide

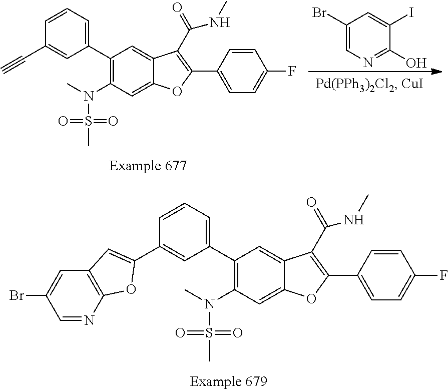

To a solution of the compound of Example 677 (150 mg, 0.32 mmol) and 5-bromo-3-iodopyridin-2-ol (105 mg, 0.35 mmol) in THF-Et₃N (1:1, 4 mL) was added CuI (10 mg) and Pd(PPh₃)₂Cl₂ (20 mg) and the reaction was allowed to stir at room temperature for 3 hours. The reaction mixture was diluted with EtOAc and filtered and the organic phase was washed with NH₄Cl, water and brine, dried over Na₂SO₄, filtered and concentrated in vacuo. The residue obtained was purified using column chromatography eluted with petroleum ether:EtOAc=2:1 to provide the title compound (100 mg, 49%). ¹H-NMR (CDCl₃, 400 MHz) δ 8.29 (s, 1H), 8.00 (br s, 2H), 7.88˜7.94 (m, 3H), 7.82 (s, 1H), 7.47˜7.61 (m, 3H), 7.15˜7.20 (m, 2H), 7.03 (s, 1H), 6.07 (br s, 1H), 3.12 (s, 3H), 2.97 (d, J=4.8 Hz, 3H), 2.73 (s, 3H). MS (M+H)⁺: 648/650.

### Example 680

### 2-(4-fluorophenyl)-5-(3-(furo[2,3-b]pyridin-2-yl)phenyl)-N-methyl-6-(N-methylmethylsulfonamido)benzofuran-3-carboxamide

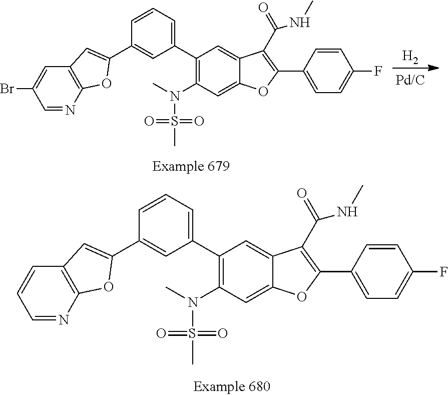

A mixture of the compound of Example 679 (30 mg, 0.05 mmol), Pd/C (10 mg, 5%) and Et₃N (0.1 mL) in MeOH (5 mL) was put under H₂ atmosphere (30 psi) and allowed to stir at room temperature for about 15 hours. The reaction mixture was filtered, the filtrate was concentrated in vacuo and the residue obtained was purified using PTLC to provide the title compound (10 mg, 38%). ¹H-NMR (CDCl₃, 400 MHz) δ 8.30 (d, J=3.6 Hz, 1H), 8.03 (s, 1H), 7.91˜7.98 (m, 4H), 7.86 (s, 1H), 7.63 (s, 1H), 7.56 (t, J=7.6 Hz, 1H), 7.49 (d, J=7.6 Hz, 1H), 7.18˜7.25 (m, 3H), 7.10 (s, 1H), 5.94 (br s, 1H), 3.17 (s, 3H), 3.00 (d, J=4.8 Hz, 3H), 2.70 (s, 3H). MS (M+H)⁺: 570.

### Example 681

### 5-(4-fluoro-1H-indol-2-yl)-2-(4-fluorophenyl)-N-methyl-6-(N-methylmethylsulfonamido)benzofuran-3-carboxamide

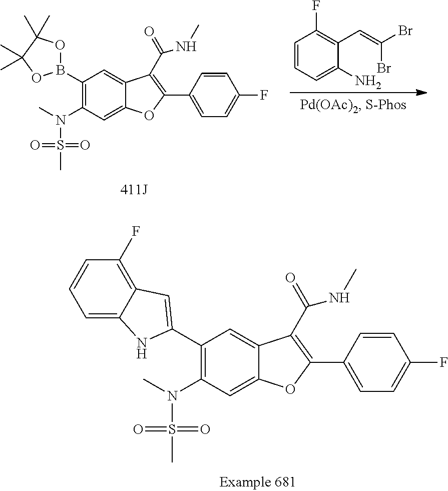

A solution of Pd(OAc)₂ (4 mg) and S-Phos (14 mg, 0.03 mmol) in toluene (2 ml) was stirred for 10 minutes under N₂ atmosphere. The reaction mixture was then added to a stirring solution of 2-(2,2-dibromovinyl)-3-fluoroaniline (50 mg, 0.17 mmol), Compound 411J (126 mg, 0.25 mmol) and K₃PO₄ (108 mg, 0.51 mmol). The resulting reaction was heated to 110° C. and allowed to stir at this temperature for 12 hours, then water was added and the solution was extracted with ethyl acetate. The organic extract was washed with brine, dried over Na₂SO₄, filtered and concentrated in vacuo and the residue obtained was purified using TLC to provide the title compound (30 mg, 35%). MS (M+H)⁺: 510. ¹H-NMR (CDCl₃, 400 MHz) δ 9.53 (s, 1H), 7.99 (s, 1H), 7.86˜7.87 (m, 2H), 7.48 (s, 1H), 7.01˜7.19 (m, 4H), 6.19˜6.74 (m, 2H), 5.83 (d, J=4.0 Hz, 1H), 3.07 (s, 3H), 3.02 (s, 3H), 2.94 (d, J=4.8 Hz, 3H). MS (M+H)⁺: 510.

### Example 682

### 5-(3-(4-fluorobenzo[b]thiophen-2-yl)phenyl)-2-(4-fluorophenyl)-N-methyl-6-(N-methylmethylsulfonamido)benzofuran-3-carboxamide

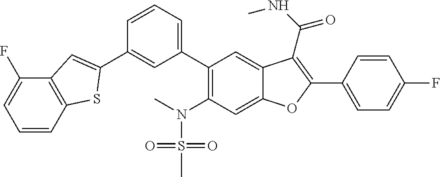

### Step 1—Synthesis of 2-(4-fluorophenyl)-N-methyl-6-(N-methylmethylsulfonamido)-5-(3-nitrophenyl)benzofuran-3-carboxamide

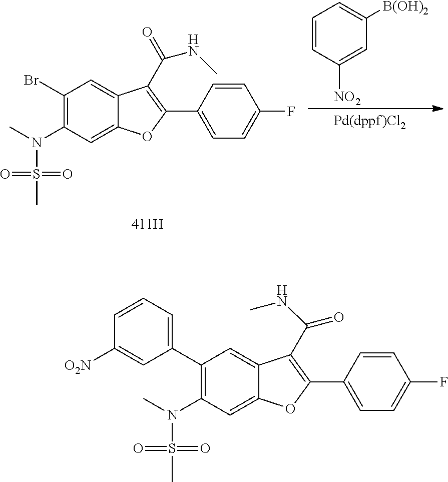

To a degassed solution of Compound 411H (2.0 g, 4.39 mmol) and 3-nitrophenylboronic acid (880 mg, 5.27 mmol) in dry DMF (1.5 mL), under nitrogen atmosphere, was added Pd(dppf)Cl₂(20 mg) and K₃PO₄ (1.86 g, 8.79 mmol). The reaction was heated to 90° C. and allowed to stir at this temperature for about 15 hours. The reaction mixture was cooled to room temperature, diluted with EtOAc and filtered, and the filtrate was washed with H₂O, brine, and dried over Na₂SO₄, filtered and concentrated in vacuo. The residue obtained was purified using flash column chromatography on silica gel (eluted with dichloromethane:EtOAc=20:1) to provide 2-(4-fluorophenyl)-N-methyl-6-(N-methylmethylsulfonamido)-5-(3-nitrophenyl)benzofuran-3-carboxamide (1.78 g, 84%). ¹H-NMR (CDCl₃, 400 MHz) δ 8.24 (s, 1H), 8.18 (d, J=8.4 Hz, 1H), 7.83˜7.87 (m, 2H), 7.79 (d, J=5.6 Hz, 1H), 7.77 (s, 1H), 7.58 (s, 1H), 7.55 (t, J=4.0 Hz, 1H), 7.15 (t, J=8.8 Hz, 2H), 5.83 (d, J=3.2 Hz, 1H), 3.09 (s, 3H), 2.92 (d, J=4.8 Hz, 3H), 2.73 (s, 3H).

### Step 2—Synthesis of 5-(3-aminophenyl)-2-(4-fluorophenyl)-N-methyl-6-(N-methylmethylsulfonamido)benzofuran-3-carboxamide

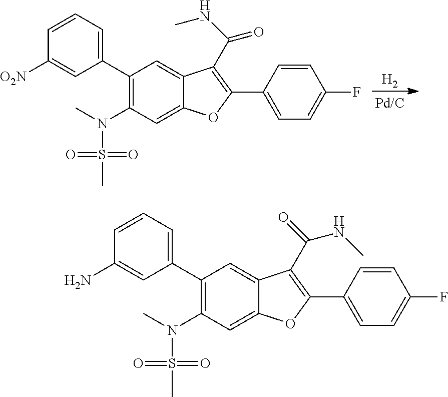

To a solution of 2-(4-fluorophenyl)-N-methyl-6-(N-methylmethylsulfonamido)-5-(3-nitrophenyl)benzofuran-3-carboxamide (1.0 g, 2.01 mmol) in MeOH (30 mL), Pd/C (200 mg) was added and the resulting reaction mixture was stirred under 40 psi of H₂ atmosphere for 24 h at 25° C. Then the reaction mixture was filtered, and the filtrate was concentrated in vacuo to provide the crude product of 5-(3-aminophenyl)-2-(4-fluorophenyl)-N-methyl-6-(N-methylmethylsulfonamido)benzofuran-3-carboxamide (846 mg, 89%). ¹H-NMR (DMSO, 400 MHz) δ 8.49 (d, J=4.8 Hz, 1H), 7.94˜7.97 (m, 2H), 7.84 (s, 1H), 7.43 (s, 1H), 7.38 (t, J=9.2 Hz, 2H), 7.03 (t, J=8.0 Hz, 1H), 6.53˜6.58 (m, 3H), 5.09 (s, 2H), 3.13 (d, J=5.6 Hz, 3H), 3.04 (s, 3H), 2.81 (s, 3H). MS (M+H)⁺: 468.

### Step 3—Synthesis of 2-(4-fluorophenyl)-5-(3-iodophenyl)-N-methyl-6-(N-methylmethylsulfonamido)benzofuran-3-carboxamide

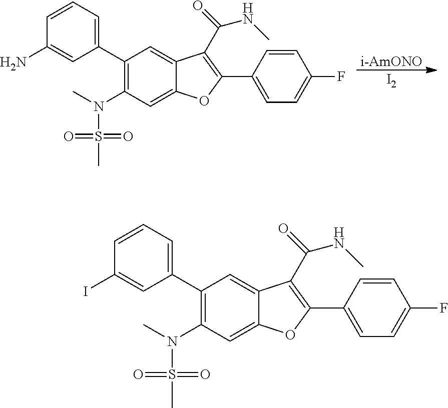

To a stirred solution of 5-(3-aminophenyl)-2-(4-fluorophenyl)-N-methyl-6-(N-methylmethylsulfonamido)benzofuran-3-carboxamide (1.5 g, 3.21 mmol) in MeCN (20 mL) was added I₂ (488.6 mg, 1.93 mmol) and CuI (6 mg) at 0° C., then i-AmONO (394.6 mg, 3.37 mmol) was added dropwise. The reaction was allowed to stir at 25° C. and allowed to stir at this temperature for 6 hours, then the reaction mixture was heated to 90° C. and allowed to stir at this temperature for 1 hour. The mixture was diluted with Na₂S₂O₃ and concentrated in vacuo to remove the organic solvent, and then the residue obtained was extracted with EtOAc. The organic layer was washed with brine, dried over Na₂SO₄ and concentrated in vacuo. The residue obtained was purified using column chromatography eluted with petroleum ether:EtOAc=3:1 then with pure dichloromethane to provide 2-(4-fluorophenyl)-5-(3-iodophenyl)-N-methyl-6-(N-methylmethylsulfonamido)benzofuran-3-carboxamide (1.17 g, 65%). ¹H-NMR (CDCl₃, 400 MHz) δ 7.85˜7.88 (m, 2H), 7.72 (d, J=7.6 Hz, 2H), 7.66 (d, J=8.0 Hz, 1H), 7.53 (s, 1H), 7.38 (d, J=7.6 Hz, 1H), 7.14 (t, J=6.0 Hz, 2H), 5.77 (d, J=4.0 Hz, 1H), 3.06 (s, 3H), 2.92 (d, J=4.8 Hz, 3H), 2.61 (s, 3H). MS (M+H)⁺: 579.

### Step 4—Synthesis of 2-(4-fluorophenyl)-N-methyl-6-(N-methylmethylsulfonamido)-5-(3-(4,4,5,5-tetramethyl-1,3,2-dioxaborolan-2-yl)phenyl)benzofuran-3-carboxamide

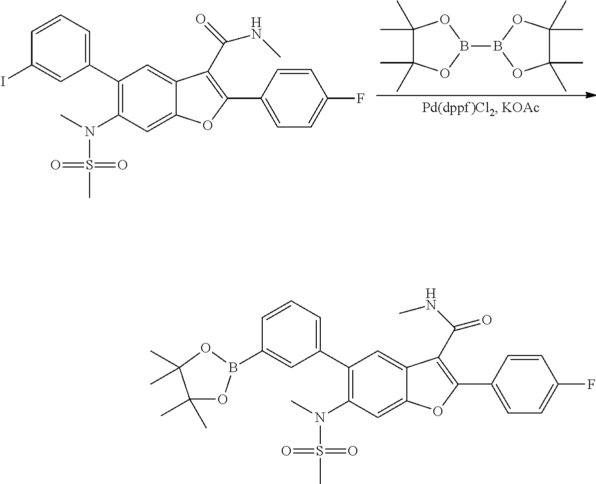

To a degassed solution of 2-(4-fluorophenyl)-5-(3-iodophenyl)-N-methyl-6-(N-methylmethylsulfonamido)benzofuran-3-carboxamide (200 mg, 0.35 mmol) and pinacol diborane (132 mg, 0.52 mmol) in dry DMF (1.5 mL) was added Pd(dppf)Cl₂(10 mg) and KOAc (102 mg, 1.04 mmol) under N₂. The mixture was heated to 90° C. and allowed to stir at this temperature for about 15 hours. The reaction mixture was cooled to room temperature and filtered. The filtrate was washed with H₂O, brine, dried over Na₂SO₄. After being concentrated in vacuo, the residue obtained was purified using column chromatography eluted with petroleum ether:EtOAc=4:1 to provide 2-(4-fluorophenyl)-N-methyl-6-(N-methylmethylsulfonamido)-5-(3-(4,4,5,5-tetramethyl-1,3,2-dioxaborolan-2-yl)phenyl)benzofuran-3-carboxamide (190 mg, 95%). ¹H-NMR (CDCl₃, 400 MHz) δ 7.88˜7.92 (m, 2H), 7.75˜7.78 (m, 2H), 7.72 (s, 1H), 7.56 (s, 1H), 7.49˜7.52 (m, 1H), 7.37˜7.41 (m, 1H), 7.11˜7.15 (m, 2H), 5.81˜5.82 (m, 1H), 3.05 (s, 3H), 2.93 (d, J=4.8 Hz, 3H), 2.51 (s, 3H), 1.29 (s, 12H). MS (M+H)⁺: 579.

### Step 5—Synthesis of 5-(3-(4-fluorobenzo[b]thiophen-2-yl)phenyl)-2-(4-fluorophenyl)-N-methyl-6-(N-methylmethylsulfonamido)benzofuran-3-carboxamide

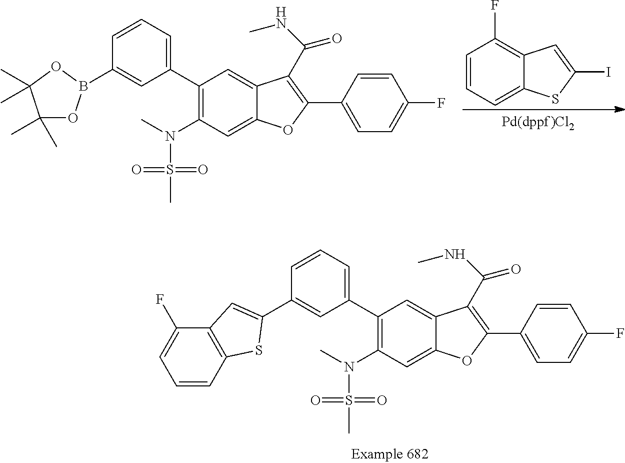

To a degassed solution of 2-(4-fluorophenyl)-N-methyl-6-(N-methylmethylsulfonamido)-5-(3-(4,4,5,5-tetramethyl-1,3,2-dioxaborolan-2-yl)phenyl)benzofuran-3-carboxamide (90 mg, 0.19 mmol) and 4-fluoro-2-iodobenzothiophene (65 mg, 0.25 mmol) in dry DMF (1.5 mL) was added Pd(dppf)Cl₂ (20 mg) and K₃PO₄ (81 mg, 0.38 mmol) under N₂. The reaction was heated to 100° C. and allowed to stir at this temperature for about 15 hours. The reaction mixture was cooled to room temperature and filtered. The filtrate was washed with H₂O, brine, dried over Na₂SO₄. After being concentrated in vacuo, the residue obtained was purified using prep-HPLC to provide the title compound (55 mg, 58.7%).

¹H-NMR (CDCl₃, 400 MHz) δ 7.86˜7.89 (m, 2H), 7.77 (s, 1H), 7.74 (s, 1H), 7.64˜7.70 (m, 2H), 7.56 (s, 1H), 7.54 (d, J=8.0 Hz, 1H), 7.43˜7.47 (m, 1H), 7.17˜7.22 (m, 1H), 7.13 (t, J=8.8 Hz, 2H), 6.95˜7.98 (m, 1H), 5.85 (d, J=4.4 Hz, 1H), 3.12 (s, 3H), 2.91 (d, =4.8 Hz, 3H), 2.59 (s, 3H). MS (M+H)⁺: 603.

The following compounds of the present invention were made using the method described above and using the appropriate reactants and/or reagents.

| | | | MS |
|---|---|---|---|
| Example | Structure | NMR | (M + H)⁺ |
| 683 | 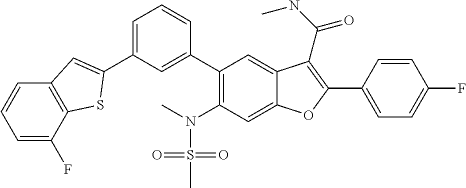 | ¹H-NMR (CDCl₃, 400 MHz) δ 7.87~7.90 (m, 2H), 7.79 (s, 1H), 7.76 (s, 1H), 7.68 (d, J = 8.0 Hz, 1H), 7.39~7.58 (m, 5H), 7.23~7.28 (m, 1H), 7.15 (t, J = 8.4 Hz, 2H), 6.96 (t, J = 8.0 Hz, 1H), 5.85 (d, J = 3.2 Hz, 1H), 3.12 (s, 3H), 2.92 (d, J = 4.8 Hz, 3H), 2.60 (s, 3H). | 603 |
| 684 | 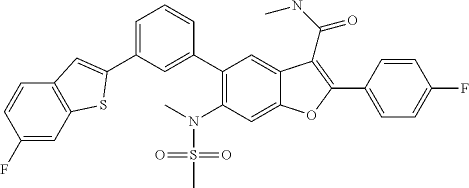 | ¹H-NMR (CDCl₃, 400 MHz) δ 7.89~7.86 (m, 2H), 7.77 (s, 1H), 7.72 (s, 1H), 7.66~7.62 (m, 2H), 7.55 (s, 1H), 7.51 (s, 1H), 7.45~7.42 (m, 2H), 7.36 (d, J = 7.6 Hz, 1H), 7.14 (t, J = 4.4 Hz, 2H), 7.06~7.01 (m, 1H), 5.83 (d, J = 4.4 Hz, 1H), 3.1 (s, 3H), 2.91 (d, J = 4.8 Hz, 3H), 2.60 (s, 3H). | 603 |
| 685 | 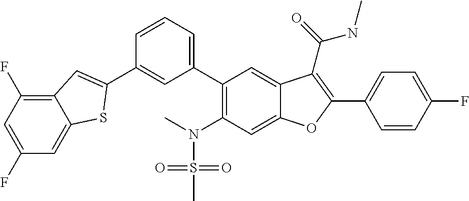 | ¹H-NMR (CDCl₃, 400 MHz) δ 7.93~7.96 (m, 2H), 7.86 (s, 1H), 7.79 (s, 1H), 7.72 (d, J = 8.0 Hz, 1H), 7.64 (d, J = 8.8 Hz, 2H), 7.52 (t, J = 7.6 Hz, 1H), 7.46 (d, J = 3.6 Hz, 1H), 7.33 (d, J = 8.8 Hz, 1H), 7.21 (t, J = 8.4 Hz, 2H), 6.86 (t, J = 8.8 Hz, 1H), 5.89 (d, J = 3.2 Hz, 1H), 3.18 (s, 3H), 2.99 (d, J = 4.8 Hz, 3H), 2.69 (s, 3H) | 621 |
| 686 | 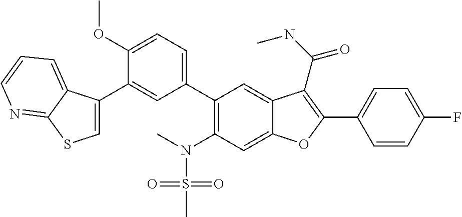 | ¹H-NMR (CDCl₃, 400 MHz) δ 8.60 (s, 1H), 8.11 (d, J = 3.6 Hz, 1H), 7.87~7.84 (m, 2H), 7.79 (s, 1H), 7.64 (s, 1H), 7.51 (s, 1H), 7.46~7.44 (m, 2H), 7.44~7.39 (m, 1H), 7.14 (t, J = 8.4 Hz, 2H), 7.06 (d, J = 4.4 Hz, 1H), 5.78 (d, J = 1.4 Hz, 1H), 3.78 (s, 3H), 3.10 (s, 3H), 2.92 (d, J = 2.4 Hz, 3H), 2.77 (s, 3H). | 616 |
| 687 | 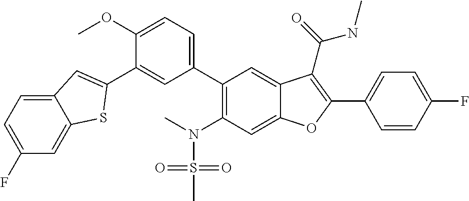 | ¹H-NMR (CDCl₃, 400 MHz) δ 8.00~7.97 (m, 2H), 7.86 (s, 1H), 7.81 (t, J = 3.0 Hz, 2H), 7.76~7.73 (m, 1H), 7.65 (s, 1H), 7.54 (t, J = 4.6 Hz, 1H), 7.47~7.45 (m, 1H), 7.23 (t, J = 9.2 Hz, 2H), 7.30 (d, J = 4.0 Hz, 2H), 5.78 (t, J = 10 Hz, 1H), 4.06 (s, 3H), 3.21 (s, 3H), 3.03 (d, J = 1.2 Hz, 3H), 2.74 (s, 3H). | 633 |
| 688 | 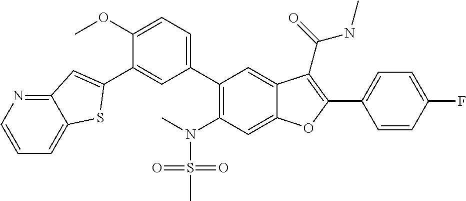 | ¹H-NMR (CDCl₃, 400 MHz) δ 8.73 (s, 1H), 8.58 (d, J = 3.8 Hz, 2H), 8.42 (s, 1H), 7.98 (s, 1H), 7.90~7.87 (m, 2H), 7.79 (s, 1H), 7.61~7.58 (m, 1H), 7.52 (s, 2H), 7.16~7.09 (m, 2H), 6.16 (d, J = 2.2 Hz, 1H), 4.04 (s, 3H), 3.10 (s, 3H), 2.94 (d, J = 2.2 Hz, 3H), 2.82 (s, 3H). | 616 |
| 689 | 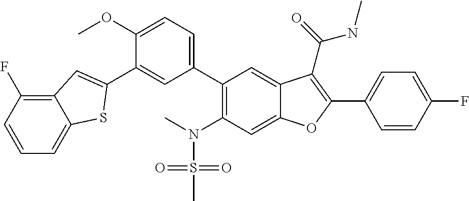 | ¹H-NMR (CDCl₃, 400 MHz) δ 7.91~7.78 (m, 2H), 7.77~7.55 (m, 2H), 7.51 (d, J = 4.0 Hz, 1H), 7.40~7.38 (m, 1H), 7.26~7.22 (m, 1H), 7.16 (s, 1H), 7.13 (d, J = 4.4 Hz, 2H), 7.05 (d, J = 4.4 Hz, 1H), 6.95~6.91 (m, 2H), 5.98 (s, 1H), 3.98 (s, 3H), 3.12 (s, 3H), 2.92 (d, J = 2.4 Hz, 3H), 2.65 (s, 3H). | 633 |

### Example 690

### 2-(4-fluorophenyl)-6-(N-(3-fluoropropyl)methylsulfonamido)-5-(3-(furo[3,2-b]pyridin-2-yl)-4-methoxyphenyl)-N-methylbenzofuran-3-carboxamide

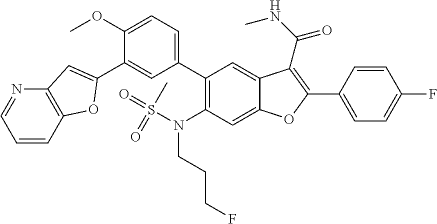

### Step 1—Synthesis of 5-bromo-2-(4-fluorophenyl)-6-(N-(3-fluoropropyl)methylsulfonamido)-N-methylbenzofuran-3-carboxamide

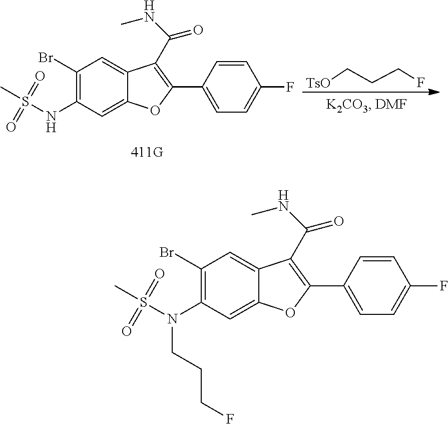

A mixture of 3-fluoropropyl 4-methylbenzenesulfonate (500 mg, 2.15 mmol) and K₂CO₃ (500 mg, 3.62 mmol) was added to a solution of Compound 411G (500 mg, 1.13 mmol) in DMF (3 mL) under N₂. The reaction was heated to 80° C. and allowed to stir at this temperature for about 15 hours. The reaction mixture was filtered and concentrated in vacuo, and the residue obtained was purified using column chromatography eluted with petroleum ether:EtOAc=3:1 to provide 5-bromo-2-(4-fluorophenyl)-6-(N-(3-fluoropropyl)methylsulfonamido)-N-methylbenzofuran-3-carboxamide (500 mg, 88%). ¹H-NMR (CDCl₃, 400 MHz) δ 8.07 (s, 1H), 7.78˜7.83 (m, 2H), 7.58 (s, 1H), 7.10˜7.13 (m, 2H), 5.90 (s, 1H), 4.40˜4.53 (m, 2H), 3.69˜3.89 (m, 2H), 3.00 (s, 3H), 2.91 (d, J=4.8 Hz, 3H), 1.85˜1.89 (m, 2H). MS (M+H)⁺: 501.

### Step 2—Synthesis of 2-(4-fluorophenyl)-6-(N-(3-fluoropropyl)methylsulfonamido)-5-(3-(furo[3,2-b]pyridin-2-yl)-4-methoxyphenyl)-N-methylbenzofuran-3-carboxamide

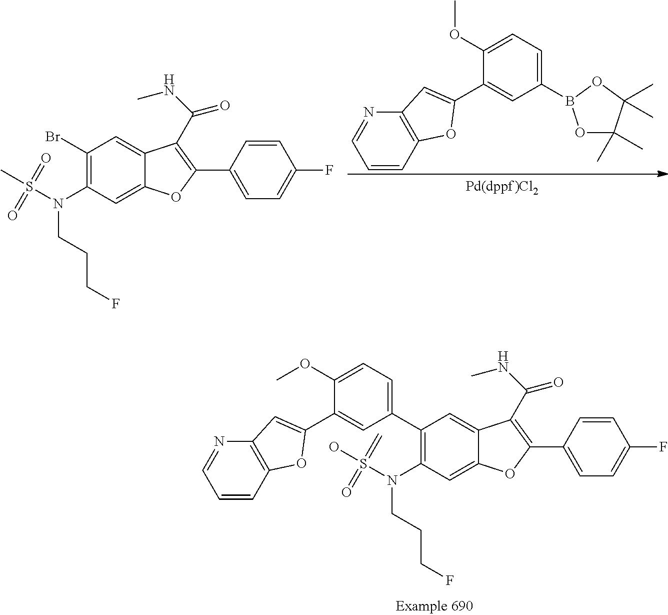

To a solution of 5-bromo-2-(4-fluorophenyl)-6-(N-(3-fluoropropyl)methylsulfonamido)-N-methylbenzofuran-3-carboxamide (100 mg, 0.2 mmol) in DMF, was added K₃PO₄ (170 mg, 0.8 mmol) and 2-(2-methoxy-5-(4,4,5,5-tetramethyl-1,3,2-dioxaborolan-2-yl)phenyl) furo[3,2-b]pyridine (100 mg, 0.28 mmol) and Pd(dppf)Cl₂ (5 mg). The reaction was put under N₂ atmosphere, heated to 80° C. and allowed to stir at this temperature for about 15 hours. The mixture was concentrated in vacuo and the residue obtained was purified using prep-HPLC to provide the title compound (30 mg, 23%). ¹H-NMR (CDCl₃, 400 MHz) δ 8.46 (d, J=4.0 Hz, 1H), 8.09 (d, J=2.4 Hz, 1H), 7.87˜7.90 (m, 2H), 7.79 (s, 1H), 7.68 (d, J=8.4 Hz, 1H), 7.53˜7.57 (m, 3H), 7.12˜7.19 (m, 3H), 7.06 (d, J=8.8 Hz, 1H), 5.91 (d, J=4.0 Hz, 1H), 4.01˜4.22 (m, 5H), 3.44˜3.48 (m, 2H), 2.91 (m, 6H), 1.62˜1.77 (m, 2H). MS (M+H)⁺: 646.

The following compounds of the present invention were made using the method described above and using the appropriate reactants and/or reagents.

| | | | MS |
|---|---|---|---|
| Example | Structure | NMR | (M + H)⁺ |
| 691 | 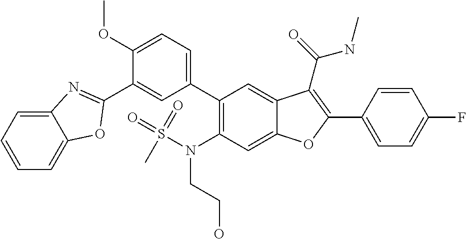 | ¹H~NMR (CDCl₃, 400 MHz) δ 8.21 (s, 1H), 7.90~7.93 (m, 2H), 7.80~7.82 (m, 3H), 7.59~7.64 (m, 2H), 7.35 (d, J = 2.8 Hz, 2H), 7.14 (t, J = 8.0, 3H), 6.08 (s, 1H),4.06 (s, 3H), 3.48~3.71 (m, 4H), 2.97 (s, 6H), 1.83 (br s, 1H). | 630 |
| 692 | 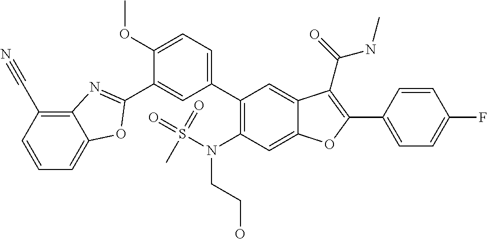 | ¹H~NMR (CDCl₃, 400 MHz) δ 8.28 (s, 1H), 7.78~7.88 (m, 5H), 7.62 (d, J = 5.6 Hz, 2H), 7.39 (t, J = 8.4 Hz, 1H), 7.12 (t, J = 8.4 Hz, 3H), 6.33 (d, J = 3.2 Hz, 1H), 4.02(s, 3H), 3.36~3.70 (m, 4H), 3.04 (s, 3H), 2.96 (d, J = 4.4 Hz, 3H), 2.21 (s, 1H). | 655 |
| 693 | 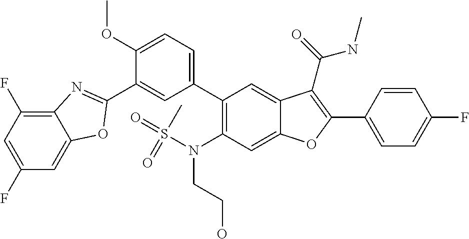 | ¹H~NMR (CDCl₃, 400 MHz) δ 8.18 (s, 1H), 7.81~7.90 (m, 4H), 7.63 (s, 1H), 7.14~7.16 (m, 4H), 6.84~6.88 (m, 1H), 6.23 (s, 1H), 4.02 (s, 3H), 3.34~3.69 (m, 4H), 3.03 (s, 3H), 2.09 (d, J = 3.2 Hz, 3H). | 666 |
| 694 | 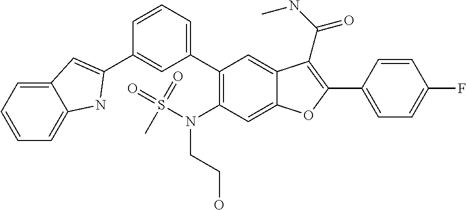 | ¹H-NMR (CDCl₃, 400 MHz) δ 9.38 (s, 1H), 8.09 (s, 1H), 7.91~7.95 (m, 2H), 7.88 (s, 1H), 7.75 (d, J = 7.6 Hz, 1H), 7.60~7.64 (m, 2H), 7.50 (t, J = 8.0 Hz, 1H), 7.43 (d, J = 8.0 Hz, 1H), 7.35 (d, J = 7.6 Hz, 1H), 7.16~7.22 (m, 3H), 7.08~7.11 (m, 1H), 6.84 (s, 1H), 5.89 (d, J = 4.4 Hz, 1H), 3.63~3.66 (m, 1H), 3.47 (m, 2H), 3.22 (s, 3H), 2.98~3.04 (m, 1H), 2.98 (d, J = 4.8 Hz, 1H). | 598 |
| 695 | 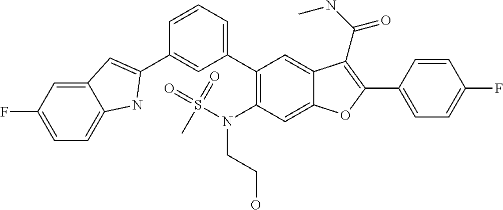 | ¹H-NMR (CDCl₃, 400 MHz) δ 7.97~7.94 (m, 2H), 7.84 (t, J = 2.0 Hz, 2H), 7.64 (t, J = 7.2 Hz, 2H), 7.36~7.31 (m, 2H), 7.25~7.13 (m, 5H), 6.86 (s, 1H), 5.96 (s, 2H), 5.88 (d, J = 4.0 Hz, 1H), 3.15(s, 3H), 2.99 (d, J = 5.2 Hz, 3H), 2.77 (s, 3H). | 616 |
| 696 | 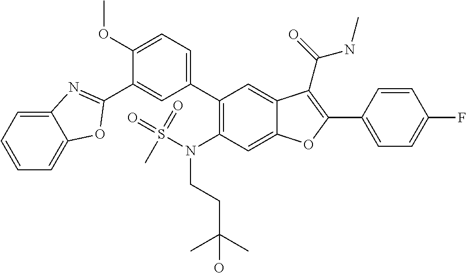 | ¹H~NMR (CDCl₃, 400 MHz) δ 8.23 (s, 1H), 7.94~7.98 (m, 2H), 7.84 (s, 2H), 7.71 (d, J = 7.2 Hz, 1H), 7.60 (d, J = 7.6 Hz, 2H), 7.37 (d, J = 4.0 Hz, 2H), 7.18~7.23 (m, 3H), 5.90 (d, J = 4.0 Hz, 1H), 4.10 (s, 3H), 3.62~3.70 (m, 2H), 2.99 (d, J = 8.0 Hz, 3H), 2.79 (s, 3H), 1.76 (t, J = 8.4 Hz, 2H), 1.25 (s, 6H). | 672 |
| 697 | 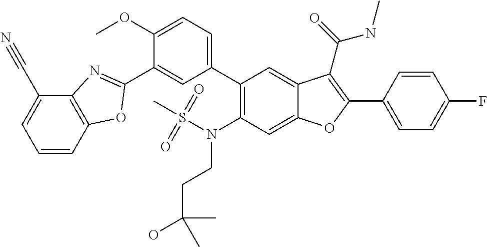 | ¹H~INIMR (CDCl₃, 400 MHz) δ 8.34 (s, 1H), 7.92~7.96 (m, 2H), 7.83 (d, J = 9.1 Hz, 2H), 7.75 (d, J = 8.0 Hz, 1H), 7.65 (d, J = 8.0 Hz, 1H), 7.62 (s, 1H), 7.42 (t, J = 8.3 Hz, 1H), 7.17~7.22 (m, 3H), 5.97 (s, 1H), 4.08 (s, 3H), 3.65 (d, J = 12.0 Hz, 1H), 3.52 (t, J = 4.0 Hz, 1H), 2.99 (d, J = 3.8 Hz, 3H), 2.94 (s, 3H), 2.04 (s, 1H), 1.25 (s, 2H), 1.14 (s, 6H). | 697 |
| 698 | 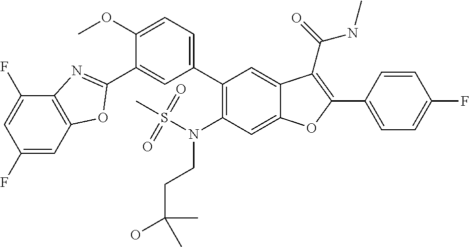 | ¹H~NMR (CDCl₃, 400 MHz) δ 8.19 (d, J = 1.7 Hz, 1H), 7.92~7.95 (m, 2H), 7.83 (s, 1H), 7.71~7.73 (m, 1H), 7.60 (s, 1H), 7.15~7.22 (m, 4H), 6.85~6.90 (m, 1H), 5.94 (d, J = 3.2 Hz, 1H), 4.07 (s, 3H), 3.65 (d, J = 5.9 Hz, 1H), 3.54 (d, J = 4.2 Hz, 1H), 3.00 (d, J = 4.6 Hz, 3H), 2.86 (s, 3H), 1.25 (s, 2H), 1.13 (d, J = 4.5 Hz, 6H). | 708 |
| 699 | 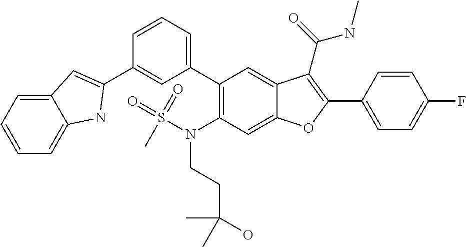 | ¹H-NMR (CDCl₃, 400 MHz) δ 9.35 (s, 1H), 7.94~7.97 (m, 3H), 7.88 (s, 1H), 7.75~7.77 (d, J = 8.0 Hz, 1H), 7.62~7.64 (d, J = 8.0 Hz, 1H), 7.48~7.52 (m, 3H), 7.41~7.43 (d, d, J = 8.0 Hz, 1H), 7.35~7.37 d, J = 8.0 Hz, 1H), 7.18~7.23 (m, 3H), 7.08~7.12 (t, d, J = 8.0 Hz, 1H), 6.85 (s, 1H), 5.87~5.88 (d, J = 4.8 Hz, 1H), 3.55~3.60 (m, 1H), 3.20~3.25 (m, 1H), 3.04 (s, 3H), 2.99~3.00 (d, J = 4.8 Hz, 3H), 1.26 (s, 2H), 1.07 (s, 3H), 1.05 (s, 3H). | 640 |
| 700 | 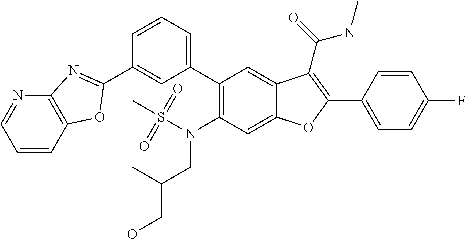 | ¹H~NMR (CDCl₃, 400 MHz) δ 8.56 (s, 1H), 8.38 (d, J = 7.2 Hz, 1H), 8.26~8.30 (m, 1H), 7.88~7.93 (m, 4H), 7.82~7.84 (m, 1H), 7.58~7.62 (m, 2H), 7.32~7.34 (m, 1H), 7.14~7.18 (m, 2H), 5.84 (s, 1H), 3.49~3.58 (m, 2H), 3.30~3.32 (m, 2H), 2.92~3.08 (m, 6H), 1.50~1.59 (m, 1H), 0.78 (d, J = 6.0 Hz, 3H), 0.54 (d, J = 6.4 Hz, 1H). | 629 |
| 701 | 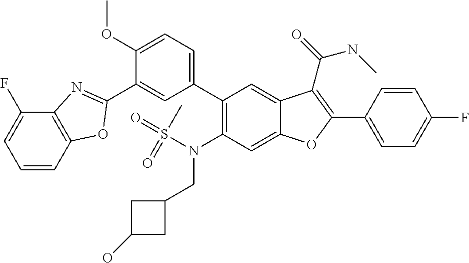 | ¹H~NMR (MeOD, 400 MHz) δ 8.30 (d, J = 2.4 Hz, 1H), 7.96~8.00 (m, 2H), 7.87~7.89 (m, 1H), 7.81 (s, 1H), 7.71 (s, 1H), 7.52 (s, 1H), 7.39~7.43 (m, 1H), 7.33~7.35 (m, 1H), 7.24~7.29 (m, 2H), 7.13~7.18 (m, 1H), 4.98 (s, 2H), 4.06 (s, 3H), 3.77~3.78 (m, 1H), 3.46 (s, 3H), 3.11~3.13 (m, 4H), 2.93 (s, 3H), 1.99~2.01 (m, 1H). | 688 |
| 702 | 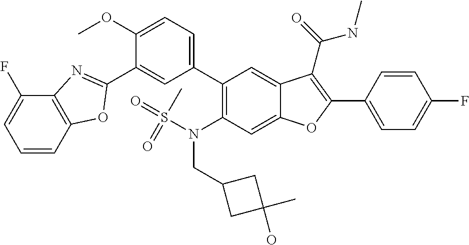 | ¹H-NMR (MeOD, 400 MHz) δ 8.28 (d, J = 2.4 Hz, 1H), 7.96~8.00 (m, 2H), 7.89~7.92 (m, 1H), 7.83 (s, 1H), 7.72 (s, 1H), 7.49~7.51 (m, 1H), 7.39~7.42 (m, 2H), 7.24~7.35 (m, 2H), 7.13~7.17 (m, 1H), 4.06 (s, 3H), 3.54~3.58 (m, 2H), 3.47~3.51 (s, 3H), 3.14 (s, 3H), 2.93 (s, 3H), 1.43~1.45 (m, 2H), 1.03 (s,3H). | 702 |
| 703 | 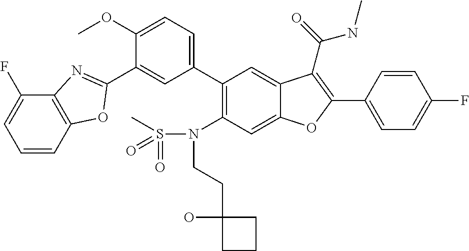 | ¹H-NMR (CDCl₃, 400 MHz): δ 8.24 (s, 1H), 7.19~7.98 (m, 2H), 7.62 (s, 1H), 7.39~7.42 (m, 1H), 7.34 (s, 1H), 7.04~7.32 (m, 6H), 5.93 (br, 1H), 4.08 (s, 3H), 3.48~3.52 (m, 2H), 3.00 (d, J = 5.1 Hz, 3H), 2.87 (s, 3H), 1.41~1.92 (m, 8H). | 702 |
| 704 | 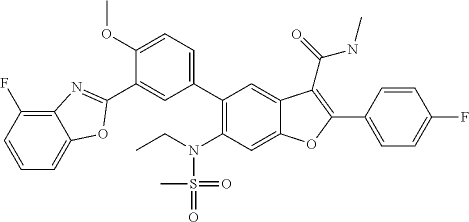 | ¹H~NMR(CDCl₃, 400 MHz)δ 8.16 (d, J = 1.0 Hz, 1H), 7.90~7.86 (m, 2H), 7.77 (s, 1H), 7.71~7.68 (m, 1H), 7.51 (s, 1H), 7.34 (d, J = 4.2 Hz, 1H), 7.25~7.23 (m, 1H), 7.22~7.10 (m, 3H), 7.08~6.98 (m, 1H), 5.95 (d, J = 2.4 Hz, 1H), 4.00 (s, 3H), 3.48 (d, J = 5.2 Hz, 2H), 3.34 (t, J = 6.8 Hz, 3H), 2.93 (t, J = 4.8 Hz, 3H), 0.99 (t, J = 7.2 Hz, 3H). | 632 |
| 705 | 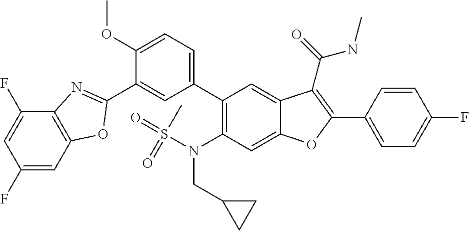 | ¹H~NMR (MeOD, 400 MHz) δ 8.28 (s, 1H), 7.96~8.00 (m, 2H), 7.85~7.87 (m, 2H), 7.71 (s, 1H), 7.24~7.38 (m, 4H), 7.04~7.09 (m, 1H), 4.60 (s, 2H), 4.04 (s, 3H), 3.07 (s, 3H), 2.93 (s, 3H), 0.84~0.88 (m, 1 H), 0.33~0.42 (s, 2H), 0.02 (s, 2H). | 676 |
| 706 | 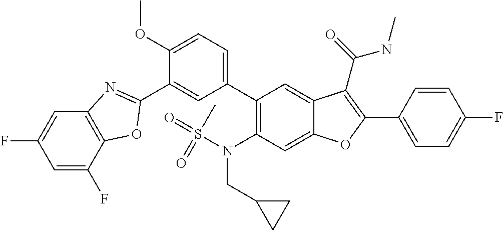 | ¹H~NMR (MeOD, 400 MHz) δ 8.33 (s, 1H), 7.96~8.00 (m, 2H), 7.85~7.88 (m, 2H), 7.71 (s, 1H), 7.24~7.35 (m, 4H), 7.09~7.14 (m, 1H), 4.80 (s, 2H), 4.06 (s, 3H), 3.05 (s, 3H), 2.93 (s, 3H), 0.87~0.89 (m, 1H), 0.37~0.43 (m, 2H), 0.01 (s, 2H). | 676 |
| 707 | 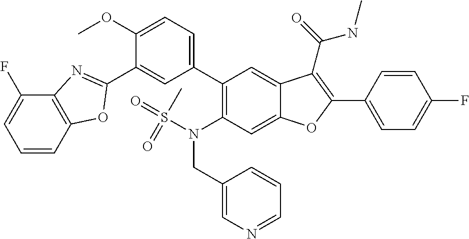 | ¹H~NMR (CDCl₃, 400 MHz) δ 8.31 (s, 1H), 8.14(s, 1H), 7.88~7.91 (m, 3H), 7.74 (s, 1H), 7.60 (d, J = 7.2 Hz, 1H), 7.35~7.41 (m, 2H), 7.28~7.31 (m, 1H), 7.26~7.27 (m, 1H), 7.10~7.18 (m, 2H), 7.03~7.07 (m, 3H), 6.09 (d, J = 4.0 Hz, 1H), 4.41~4.49 (m, 2H), 4.02 (s, 3H), 3.02 (s, 3H), 2.97 (d, J = 4.8 Hz, 3H). | 695 |
| 708 | 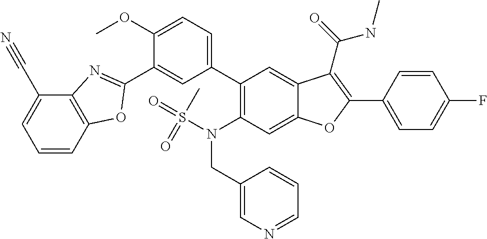 | ¹H~NMR (CDCl₃, 400 MHz) δ 8.28 (s, 1H), 8.09 (s, 1H), 7.93 (d, J = 2.4 Hz, 1H), 7.84~7.87 (m, 2H), 7.79 (d, J = 8.4 Hz, 1H), 7.71 (s, 1H), 7.61 (d, J = 7.6 Hz, 1H), 7.52~7.55 (m, 1H), 7.43 (s, 1H), 7.37 (d, J = 8.4 Hz, 1H), 7.29 (d, J = 8.4 Hz, 1H), 7.10~7.15 (m, 2H), 7.03~7.05 (m, 2H), 5.94 (d, J = 4.8 Hz, 1H), 4.38~4.44 (m, 2H), 4.02 (s, 3H), 3.07 (s, 3H), 2.91 (d, J = 4.8 Hz, 3H). | 702 |
| 709 | 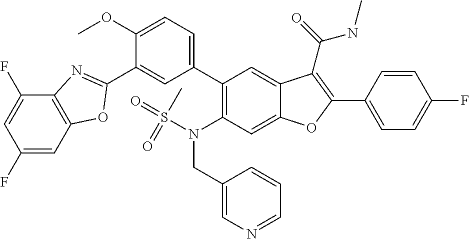 | ¹H~NMR (CDCl₃, 400 MHz) δ 8.25 (s, 1H), 8.10 (s, 1H), 7.79~7.85 (m, 3H), 7.69 (s, 1H), 7.55 (d, J = 4.0 Hz, 1H), 7.36 (s, 1H), 7.27 (d, J = 8.0 Hz, 1H), 7.11~7.13 (m, 3H), 6.96~7.09 (m, 2H), 6.82 (d, J = 8.0 Hz, 1H), 6.06 (d, J = 4.0 Hz, 1H), 4.38~4.42 (m, 2H), 3.99 (s, 3H), 2.96 (s, 3H), 2.91 (d, J = 4.0 Hz, 3H). | 713 |
| 710 | 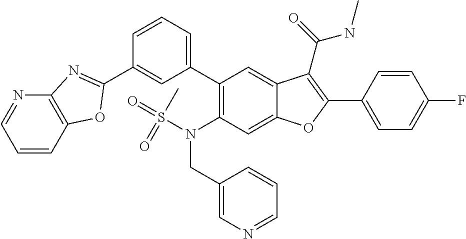 | ¹H~NMR (CDCl₃, 400 MHz) δ 8.61 (d, J = 4.0 Hz, 1H), 8.38 (s, 1H), 8.27 (d, J = 8.0 Hz, 1H), 8.17 (s, 1H), 7.96~8.00 (m, 3H), 7.90 (s, 1H), 7.85 (d, J = 8.0 Hz, 1H), 7.81 (s, 1H), 7.74 (d, J = 7.2 Hz, 1H), 7.59~7.61 (m, 2H), 7.46~7.47 (m, 1H), 7.41~7.44 (m, 1H), 7.19~7.23 (m, 2H), 6.34~6.35 (m, 1H), 4.52~4.74 (m, 2H), 3.21 (s, 3H), 2.98 (d, J = 4.0 Hz, 3H). | 648 |
| 711 | 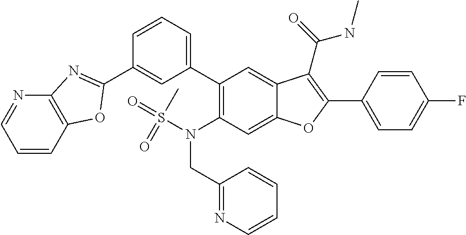 | ¹H~NMR (CDCl₃, 400 MHz) δ 8.58 (s, 1H), 8.43 (d, J = 4.0 Hz, 1H), 8.21 (d, J = 4.4 Hz, 1H), 7.97 (s, 1H), 7.84~7.91 (m, 3H), 7.67~7.70 (m, 3H), 7.56 (s, 1H), 7.47~7.51 (m, 1H), 7.26~7.33 (m, 3H), 7.11~7.15 (m, 2H), 6.07 (d, J = 4.4 Hz, 1H), 4.76 (s, 2H), 3.18 (s, 3H), 2.98 (d, J = 4.8 Hz, 3H). | 648 |
| 712 | 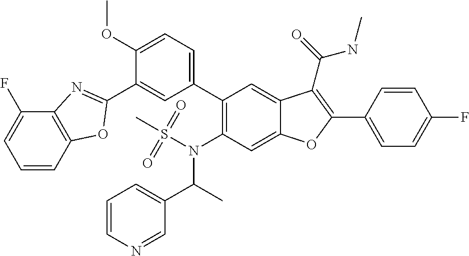 | ¹H~NMR (CDCl₃, 400 MHz) δ 8.21~8.45 (m, 1H), 7.77~8.00 (m, 4H), 7.35~7.28 (m, 5H), 7.01~7.24 (m, 4H), 6.72~6.88 (m, 2H), 6.22~6.30 (m, 1H), 5.02~5.04 (m, 1H), 3.97~4.03 (m, 3H), 2.90~3.05 (m, 6H), 0.91~1.52 (m, 3H). | 709 |
| 713 | 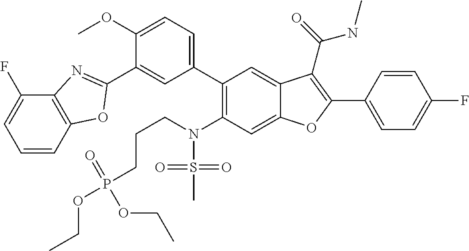 | ¹H~NMR (CDCl₃, 400 MHz) δ 8.14 (d, J = 2.4 Hz, 1H), 7.82~7.85 (m, 2H), 7.74 (s, 1H), 7.65~7.68 (m, 1H), 7.50 (s, 1H), 7.34 (d, J = 8.4 Hz, 1H), 7.20~7.23 (m, 1H), 7.05~7.10 (m, 3H), 6.98~7.10 (m, 1H), 6.33 (d, J = 4.4 Hz, 1H), 3.99 (s, 3H), 3.87~3.88 (m, 4H), 3.42~3.44 (m, 1H), 3.21~3.22 (m, 1H), 2.98 (s, 3H), 2.94~2.95 (m, 3H), 1.54~1.64 (m, 2H), 1.38~1.40 (m, 2H), 1.10~1.12 (m, 6H). | 782 |
| 714 | 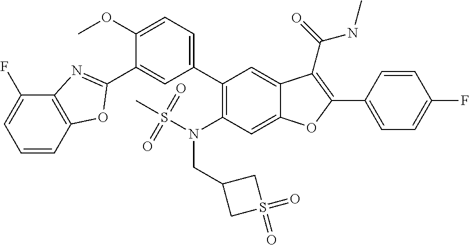 | ¹H~NMR (CDCl₃, 400 MHz) δ 8.23 (d, J = 1.2 Hz, 1H), 7.89~7.84 (m, 3H), 7.75 (t, J = 4.8 Hz, 1H), 7.58 (s, 1H), 7.41 (d, J = 8.0 Hz, 1H), 7.33~7.28 (m, 1H), 7.17~7.13 (m, 3H), 7.06 (t, J = 8.8 Hz, 1H), 6.33 (m, 1H), 4.14~4.06 (m, 4H), 3.71 (t, J = 10.0 Hz, 1H), 3.61~3.54 (m, 2H), 3.41 (s, 2H), 3.19 (s, 3H), 3.01 (d, J = 4.8 Hz, 3H), 2.34 (s, 1H). | 722 |
| 715 | 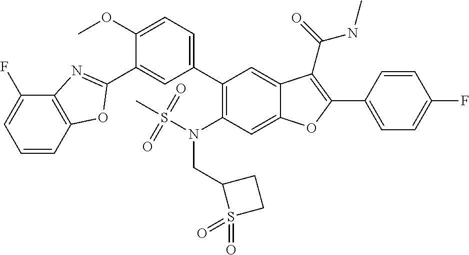 | ¹H~NMR (CDCl₃, 400 MHz) δ 8.22 (s, 1H), 7.70~7.94 (m, 4H), 7.56~7.65 (m, 1H), 7.42 (d, J = 8.0 Hz, 1H), 7.30 (d, J = 4.4 Hz, 1H), 7.17 (t, J = 8.4 Hz, 3H), 7.06 (t, J = 8.4 Hz, 1H), 6.17 (d, J = 3.6 Hz, 1H), 3.81~4.58 (m, 8H), 3.00~3.33 (m, 6H), 1.53~1.90 (m, 2H). | 722 |
| 716 | 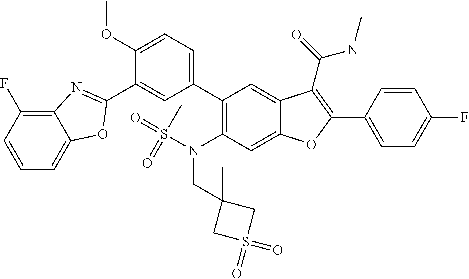 | ¹H~NMR (CDCl₃, 400 MHz) δ 8.29 (s, 1H), 7.87~8.29 (m, 3H), 7.79~7.81 (m, 1H), 7.72 (s, 1H), 7.42 (d, J = 8.0 Hz, 1H), 7.26~7.33 (m, 1H), 7.17~7.21 (m, 3H), 7.04~7.09 (m, 1H), 6.13 (s, 1H), 4.08 (s, 3H), 3.75~3.79 (m, 1H), 3.61~3.65 (m, 1H), 3.49~3.57 (m, 3H), 3.37~3.40 (m, 1H), 3.25 (s, 3H), 2.99~3.00 (m, 3H), 1.15 (s, 3H). | 736 |
| 717 | 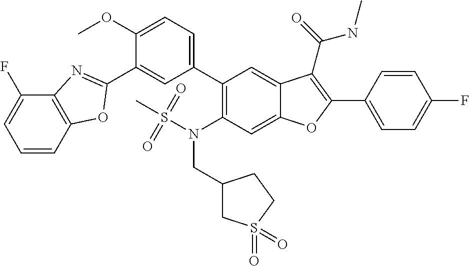 | ¹H~NMR (CDCl₃, 400 MHz) δ 8.25~8.28 (m, 1H), 7.59~7.96 (m, 5H), 7.42 (d, J = 8.4 Hz, 1H), 7.28~7.33 (m, 1H), 7.18~7.23 (m, 3H), 7.07 (d, J = 0.8 Hz, 1H), 5.96 (s, 1H), 4.08 (s, 3H), 3.48 (t, J = 5.2 Hz, 2H), 3.13~3.19 (m, 3H), 2.64~3.01 (m, 6H), 2.38~2.41 (m, 2H), 1.63~2.16 (m, 2H). | 736 |
| 718 | 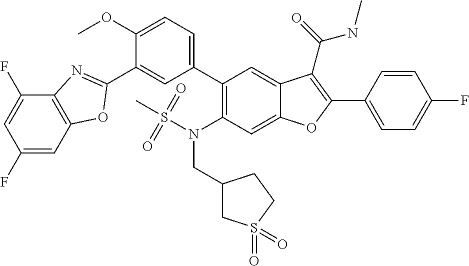 | ¹H~NMR (CDCl₃,400 MHz) δ 8.25~8.28 (m, 1H), 7.59~7.96 (m, 5H), 7.42 (d, J = 8.4 Hz, 1H), 7.28~7.33 (m, 1H), 7.18~7.23 (m, 3H), 7.07 (d, J = 0.8 Hz, 1H), 5.96 (s, 1H), 4.08 (s, 3H), 3.48(t, J = 5.2 Hz, 2H), 3.13~3.19 (m, 3H), 2.64~3.01 (m, 6H), 2.38~2.41 (m, 2H), 1.63~2.16 (m, 2H). | 754 |
| 719 | 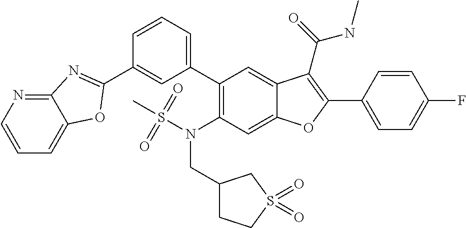 | ¹H~NMR (CDCl₃, 400 MHz) δ 8.59 (s, 1H), 8.44 (s, 1H), 8.34 (m, J = 5.6 Hz, 1H), 7.84~7.96 (m, 5H), 7.63~7.68 (m, 2H), 7.35 (t, J = 4.8 Hz, 1H), 7.19~7.33 (m, 2H), 6.01 (s, 1H), 3.47 (s, 2H), 3.11~3.14 (m, 3H), 2.58~2.99 (m, 6H), 1.57~2.35 (m, 4H). | 689 |
| 720 | 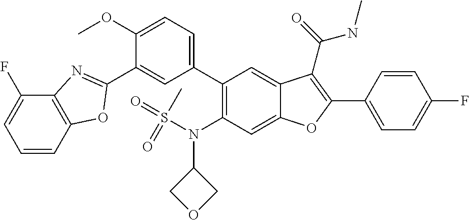 | ¹H~NMR (CDCl₃, 400 MHz) δ 8.17 (s, 1H), 7.84~7.87 (m, 3H), 7.66 (d, J = 8.0 Hz, 1H), 7.47 (s, 1H), 7.33 (d, J = 8.0 Hz, 1H), 6.99~7.25 (m, 5H), 6.08 (br s, 1H), 4.96~4.99 (m, 1H), 4.48~4.62 (m, 4H), 4.02 (s, 3H), 2.94 (d, J = 3.6 Hz, 3H), 2.68 (s, 3H). | |
| 721 | 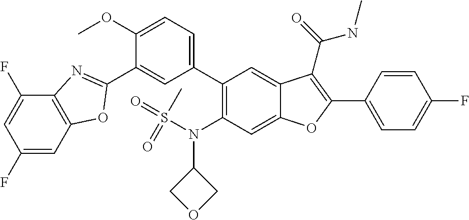 | ¹H~NMR (CDCl₃, 400 MHz) δ 8.15 (d, J = 2.4 Hz, 1H), 7.83~7.87 (m, 3H), 7.65~7.68 (m, 1H), 7.46 (s, 1H), 7.08~7.15 (m, 4H), 6.78~6.83 (m, 1H), 6.02 (d, J = 4.8 Hz, 1H), 4.97 (m, 1H), 4.56~4.62 (m, 3H), 4.49~4.51 (m, 1H), 4.00 (s, 3H) 2.93 (s, 3H), 2.69 (s, 3H). | |
| 722 | 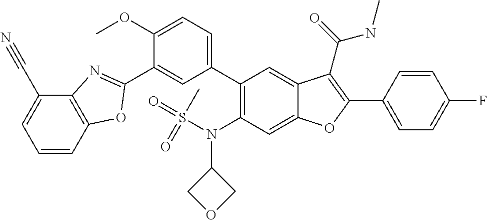 | ¹H~NMR (CDCl₃, 400 MHz) δ 8.27 (d, J = 2.0 Hz, 1H), 7.84~7.89 (m, 3H), 7.77 (d, J = 8.4 Hz, 1H), 7.69~7.71 (m, 1H), 7.59 (d, J = 8.0 Hz, 1H), 7.48 (s, 1H), 7.33~7.37 (m, 1H), 7.11~7.19 (m, 3H), 6.00 (d, J = 4.4 Hz, 1H), 4.97~5.00 (m, 1H), 4.52~4.64 (m, 4H), 4.02 (s, 3H), 2.93 (d, J = 4.8 Hz, 3H), 2.76 (s, 3H). | 667 |
| 723 | 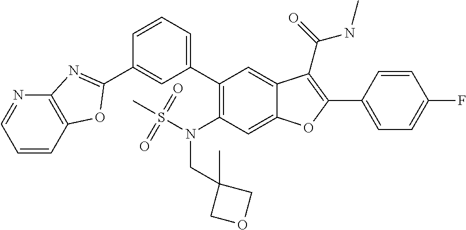 | ¹H~NMR (MeOD, 400 MHz) δ 8.52~8.56 (m, 2H), 8.38 (s, 1H), 8.16 (s, 1H), 8.00~8.09 (s, 3H), 7.73 (s, 2H), 7.48 (s, 1H), 7.28 (s, 2H), 4.04 (s, 2H), 3.95 (s, 1H), 3.82~3.89 (m, 2H), 3.68~3.72 (m, 1H), 3.23 (s, 3H), 2.92 (s, 3H), 0.91 (s, 3H). | 641 |
| 724 | 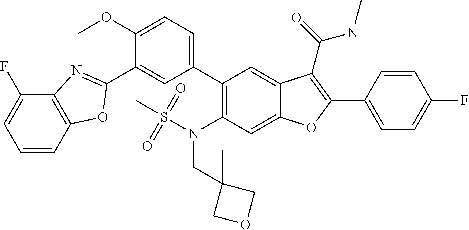 | ¹H~NMR (MeOD, 400 MHz) δ 8.31 (s, 1H), 7.93~7.97 (m, 3H), 7.71 (s, 1H), 7.60~7.65 (m, 1H), 7.50~7.52 (m, 1H), 7.35~7.42 (m, 2H), 7.24~7.29 (m, 2H), 7.13~7.17 (m, 1H), 4.02~4.09 (m, 5H), 3.88~3.94 (m, 2H), 3.81 (d, J = 14.4 Hz, 1H), 3.70 (d, J = 14.4 Hz, 1H), 3.24 (s, 3H), 2.92 (s, 3H), 0.96 (s, 3H). | 688 |
| 725 | 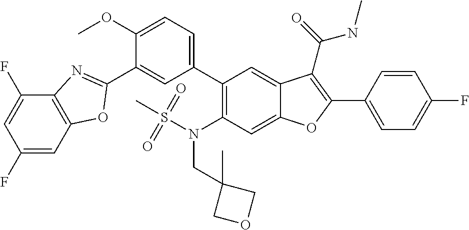 | ¹H~NMR (MeOD, 400 MHz) δ 8.29 (s, 1H), 7.92~8.29 (m, 4H), 7.70 (s, 1H), 7.34~7.39 (m, 2H), 7.24~7.29 (m, 2H), 7.04~7.10 (m, 1H), 4.01~4.06 (m, 5H), 3.87~3.93 (m, 2H), 3.81 (d, J = 14.4 Hz, 1H), 3.69 (d, J = 14.4 Hz, 1H), 3.23 (s, 3H), 2.92 (s, 3H), 0.95 (s, 3H). | 706 |
| 726 | 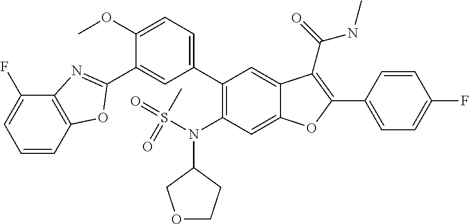 | ¹H~NMR (CDCl₃, 400 MHz) δ 8.20 (s, 1H), 7.75~8.85 (m, 2H), 7.74 (d, J = 2.4 Hz, 1H), 7.72 (t, J = 2.4 Hz, 1H), 7.49 (d, J = 9.6 Hz, 1H), 7.36 (d, J = 4.8 Hz, 1H), 7.24~7.27 (m, 1H), 7.01~7.14 (m, 4H), 6.02~6.06 (m, 1H), 4.34~4.35 (m, 1H), 4.00 (s, 3H), 3.84~3.88 (m, 1H), 3.61~3.64 (m, 2H), 2.95 (s, 3H), 2.92~2.94 (m, 1H), 2.79 (s, 3H), 1.94~2.11 (m, 1H), 1.71~1.75 (m, 1H). | 674 |
| 727 | 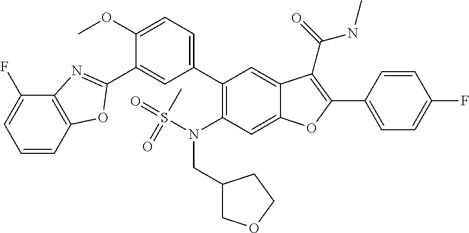 | ¹H~NMR (MeOD, 400 MHz) δ 8.24~8.26 (m, 1H), 7.90~7.94 (m, 2H), 7.81~7.84 (m, 2H), 7.69 (s, 1H), 7.46 (d, J = 8.0 Hz, 1H), 7.34~7.38 (m, 1H), 7.19~7.33 (m, 3H), 7.11 (t, J = 9.0 Hz, 1H), 4.02 (s, 3H), 3.76~3.80 (m, 1.5H), 3.65~3.70 (m, 1H), 3.43~3.53 (m, 2H), 3.32~3.37 (m, 0.5H), 3.17 (d, J = 5.6 Hz, 3H), 3.07~3.10 (m, 0.5H), 2.93 (s, 3H), 2.85~2.87 (m, 0.5H), 2.02~2.06 (m, 1H), 1.77~1.80 (m, 0.5H), 1.27~1.30 (m, 1.5H). | 706 |
| 728 | 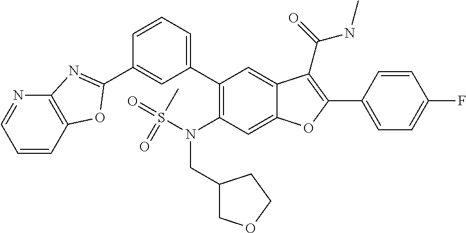 | ¹H~NMR (MeOD, 400 MHz) δ 8.50~8.55 (m, 2H), 8.33 (d, J = 8.0 Hz, 1H), 8.16 (d, J = 8.0 Hz, 1H), 7.96~7.99 (m, 2H), 7.90 (d, J = 6.8 Hz, 2H), 7.73 (s, 1H), 7.68 (t, J = 7.8 Hz, 1H), 7.46~7.49 (m, 1H), 7.26 (t, J = 8.8 Hz, 2H), 3.59~3.68 (m, 0.5H), 3.51~3.57 (m, 1H), 3.41~3.45 (m, 1H), 3.38 (d, J = 6.8 Hz, 2H), 3.23~3.25 (m, 0.5H), 3.17 (s, 3H), 3.02~3.05 (m, 0.5H), 2.92 (s, 3H), 2.79~2.83 (m, 0.5H), 2.05~2.08 (m, 1H), 1.80~1.82 (m, 0.5H), 1.33~1.37 (m, 0.5H), 1.23~1.26 (m, 1H). | 641 |
| 729 | 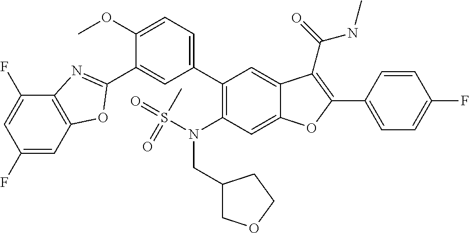 | ¹H~NMR (MeOD, 400 MHz) δ 8.21~8.23 (m, 1H), 7.88~7.91 (m, 2H), 7.80~7.82 (m, 2H), 7.67 (s, 1H), 7.29~7.32 (m, 1H), 7.18~7.24 (m, 3H), 6.98~7.03 (m, 1H), 4.00 (s, 3H), 3.50~3.66 (m, 1.5H), 3.42~3.50 (m, 1H), 3.33~3.35 (m, 2H), 3.22~3.27 (m, 0.5H), 3.16 (s, J = 4.4 Hz, 3H), 3.05~3.08 (m, 0.5H), 2.92 (s, 3H), 2.82~2.86 (m, 0.5H), 2.01~2.04 (m, 1H), 1.75~1.80 (m, 0.5H), 1.27~1.30 (m, 1.5H). | 706 |
| 730 | 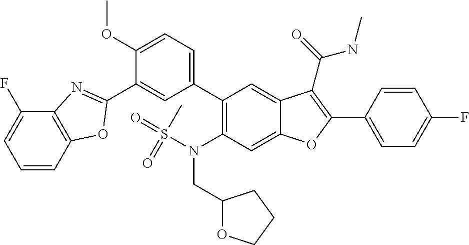 | ¹H~NMR (CDCl₃, 400 MHz) 8.23 (s, 1H), 7.94~8.04 (m, 3H), 7.84 (s, 1H), 7.63 (s, 1H), 7.27~7.42 (m, 2H), 7.15~7.18 (m, 3H), 7.05 (t, J = 8.8 Hz, 1H), 5.99 (s, 1H), 4.06 (s, 3H), 3.53~3.85 (m, 4H), 3.32 (s, 3H), 2.90 (d, J = 4.8 Hz, 3H), 2.72 (d, J = 2.0 Hz, 1H), 1.66~1.76 (m, 3 H), 1.23~1.25 (m, 1H). | 688 |
| 731 | 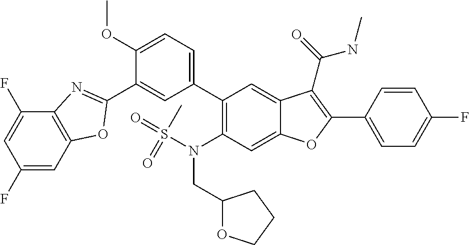 | ¹H~NMR (CDCl₃, 400 MHz) 8.15 (d, J = 8.8 Hz, 1H), 7.95~7.97 (m, 1H), 7.88~7.91 (m, 2H), 7.75 (d, J = 8.8 Hz, 1H), 7.58~7.64 (m, 1H), 7.10~7.15 (m, 4H), 6.81 (d, J = 9.6 Hz, 1H), 5.92 (s, 1H), 4.00 (s, 3H), 3.76~3.82 (m, 1H), 3.65 (t, J = 7.2 Hz, 2H), 3.45~3.56 (m, 1H), 3.27 (s, 3H), 2.93 (d, J = 3.6 Hz, 3H), 2.69~2.72 (m, 1H), 1.17~1.61 (m, 4H) | 706 |
| 732 | 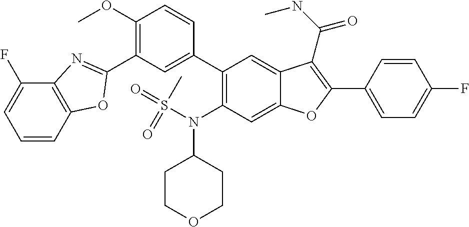 | ¹H~NMR (CDCl₃, 400 MHz) δ 8.27 (d, J = 2.0 Hz, 1H), 7.94~7.97 (m, 2H), 7.87 (t, J = 3.6 Hz, 1H), 7.85 (d, J = 2.0 Hz, 1H), 7.51 (s, 1H), 7.40 (d, J = 8.0 Hz, 1H), 7.31 (t, J = 4.4 Hz, 1H), 7.22~7.29 (m, 2H), 7.15~7.20 (m, 1H), 7.06 (t, J = 8.4 Hz, 1H), 5.89 (s, 1H), 4.06 (s, 3H), 3.90 (d, J = 7.6 Hz, 1H), 3.82~3.88 (m, 1H), 3.72 (d, J = 6.4 Hz, 1H), 3.34 (t, J = 12.0 Hz, 1H), 3.20 (t, J = 12.0 Hz, 1H), 3.14 (s, 3H), 3.00 (d, J = 4.8 Hz, 3H), 1.90 (d, J = 14.8 Hz, 2H), 1.19~1.24 (m, 2H). | 688 |
| 733 | 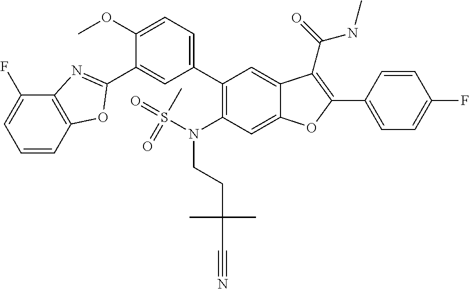 | ¹H~NMR (CDCl₃, 400 MHz) δ 8.26 (d, J = 2.0 Hz, 1H), 7.98 (q, J = 5.2, 8.4 Hz, 2H), 7.89 (s, 1H), 7.74 (dd, J = 2.0, 10.8 Hz, 1H), 7.66 (s, 1H), 7.42~7.51 (m, 1H), 7.31~7.35 (m, 1H), 7.20~7.25 (m, 3H), 7.07 (t, J = 15.6 Hz, 1H), 6.00(s, 1H), 4.12 (s, 3H), 3.55~3.71 (m, 2H), 3.04 (d, J = 4.8 Hz, 3H), 2.97 (s, 3H), 1.76~1.83 (m, 2H), 1.29 (s, 6H). | 699 |
| 734 | 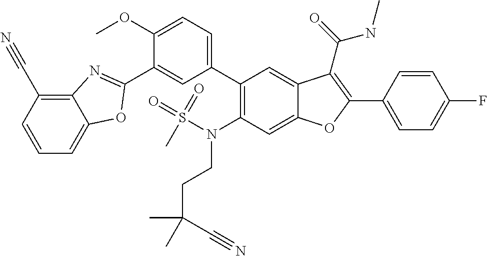 | ¹H~NMR (CDCl₃, 400 MHz) δ 8.31 (s, 1H), 7.92~7.96 (m, 2H), 7.86 (t, J = 8.8 Hz, 2H), 7.74 (d, J = 8.4 Hz, 1H), 7.66 (s,2H), 7.4 (d, J = 8.0 Hz, 1H), 7.21 (d, J = 8.0 Hz, 3H), 5.94 (s, 1H), 4.09 (s, 3H), 3.40~3.69 (m, 2H), 3.03 (s, 3H), 2.99 (d, J = 4.8 Hz, 3H), 1.69~1.78 (m, 2H), 1.27 (s, 6H). | 706 |
| 735 | 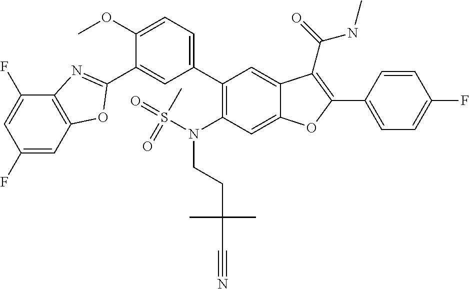 | ¹H~NMR (CDCl₃, 400 MHz) δ 8.23 (d, J = 2.6 Hz, 1H), 7.97 (q, J = 5.6, 8.8 Hz, 2H), 7.96 (s, 1H), 7.74 (dd, J = 2.0, 8.4 Hz, 1H), 7.65 (s, 1H), 7.18~7.26 (m, 4H), 6.90 (t, J = 2.0 Hz, 1H), 5.99 (s, 1H), 4.11 (s, 3H), 3.48~3.72 (m, 2H), 3.02 (d, J = 4.8 Hz, 3H), 2.97 (s, 3H), 1.77~1.83 (m, 2H), 1.29 (s, 6H). | 717 |
| 736 | 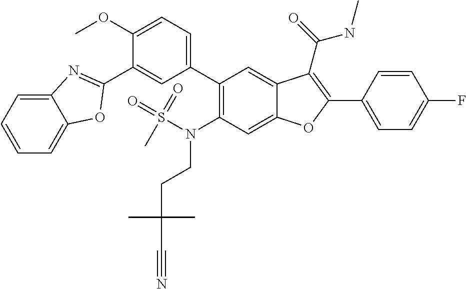 | ¹H~NMR (CDCl₃ 400 MHz) δ 8.20 (d, J = 2.0 Hz, 1H), 7.94 (q, J = 5.2, 8.0 Hz, 2H), 7.85 (s, 1H), 7.80 (t, J = 4.4 Hz, 1H), 7.70 (dd, J = 1.6 Hz, 8.8 Hz, 1H), 7.62 (s, 1H), 7.56~7.59 (m, 1H), 7.34~7.37 (m, 2H), 7.17~7.22 (m, 3H), 6.02 (s, 1H), 4.09 (s, 3H), 3.40~3.69 (m, 2H), 2.99 (d, J = 4.8 Hz, 3H), 2.93 (s, 311), 1.65~1.80 (m, 2H), 1.24 (s, 6H). | 681 |
| 737 | 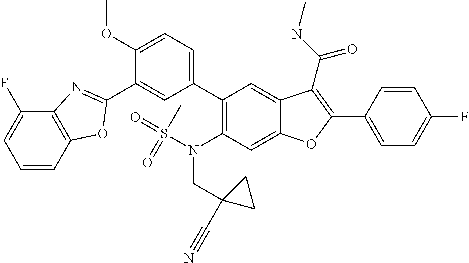 | ¹H~NMR (MeOD, 400 MHz) δ 8.31 (s, 1H), 7.97~8.00 (m, 2H), 7.91~7.93 (m, 2H) 7.73 (s, 1H), 7.32~7.37 (m, 2H), 7.24~7.29 (m, 2H), 7.03~7.14 (m, 2H), 4.64 (s, 1H), 4.04 (s, 1H), 3.67~3.71 (m, 2H), 3.21~3.25 (m, 6H), 2.93 (s, 3H), 0.92~1.12 (m, 1H), 0.59~0.76 (m, 1H). | 683 |
| 738 | 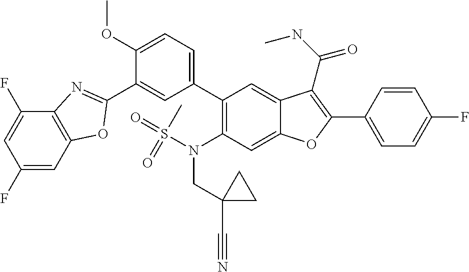 | ¹H~NMR (MeOD, 400 MHz) δ 8.31 (s, 1H), 7.97~8.00 (m, 2H), 7.91~7.93 (m, 2H) 7.73 (s, 1H), 7.32~7.37 (m, 2H), 7.24~7.29 (m, 2H), 7.03~7.14 (m, 2H), 4.64 (s, 1H), 4.04 (s, 1H), 3.67~3.71 (m, 2H), 3.21~3.25 (m, 6H), 2.93 (s, 3H), 0.92~1.12 (m, 1H), 0.59~0.76 (m, 1H). | 701 |
| 739 | 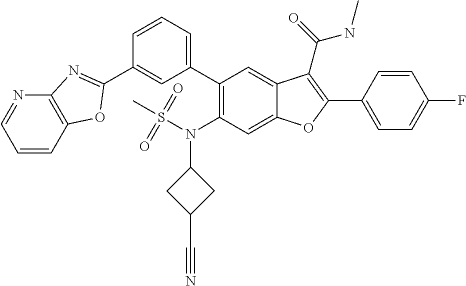 | ¹H~NMR(CDCl₃, 400 MHz) δ 8.51 (s, 1H), 8.27~8.32 (m, 2H), 7.90~7.93 (m, 4H), 7.70 (d, J = 8.0 Hz, 1H), 7.56~7.60 (m, 1H), 7.47 (s, 1H), 7.26~7.29 (m, 1H), 7.15~7.19 (m, 2H), 6.32 (d, J = 4.0 Hz, 1H), 4.25~4.30 (m, 1H), 2.98 (d, J = 4.0 Hz, 3H), 2.79 (s, 3H), 2.59~2.68 (m, 2H), 2.36~2.47 (m, 2H), 2.11~2.14 (m, 1H). | 636 |
| 740 | 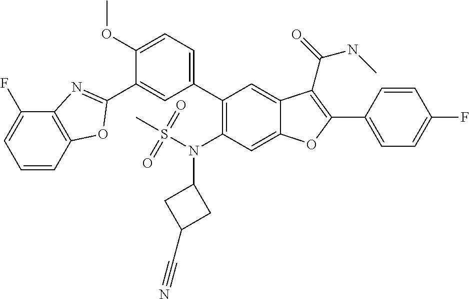 | ¹H~NMR (CDCl₃, 400 MHz) δ 8.18 (d, J = 4.0 Hz, 1H), 7.89~7.92 (m, 3H), 7.68~7.70 (m, 1H), 7.40~7.45 (m, 2H), 7.26~7.30 (m, 1H), 7.11~7.19 (m, 3H), 7.01~7.06 (m, 1H), 6.18 (d, J = 4.0 Hz, 1H), 4.21~4.30 (m, 1H), 4.01 (s, 3H), 2.98 (d, J = 4.0 Hz, 3H), 2.88 (s, 3H), 2.59~2.64 (m, 2H), 2.32~2.43 (m, 2H), 2.08~2.15 (m, 1H). | 683 |
| 741 | 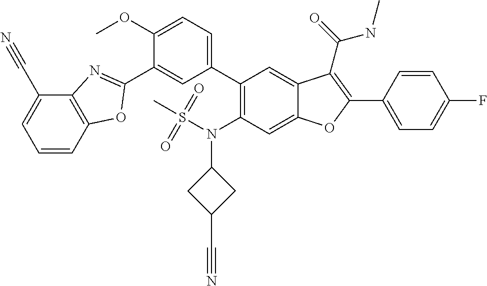 | ¹H~NMR (CDCl₃, 400 MHz) δ 8.35 (d, J = 1.2 Hz, 1H), 7.85~7.94 (m, 4H), 7.73 (d, J = 7.2 Hz, 1H), 7.65 (d, J = 7.6 Hz, 1H), 7.40~7.47 (m, 2H), 7.16~7.22 (m, 3H), 6.04 (d, J = 4.0 Hz, 1H), 4.73~4.81 (m, 1H),4.08 (s, 3H), 3.00 (d, J = 4.4 Hz, 3H), 2.89 (s, 3H), 2.43~2.57 (m, 4H). | 690 |
| 742 | 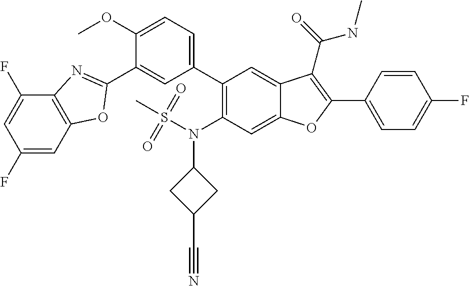 | ¹H~NMR (CDCl₃, 400 MHz) δ 8.19 (d, J = 2.0 Hz, 1H), 7.88~7.92 (m, 3H), 7.68~7.71 (m, 1H), 7.45 (s, 1H), 7.13~7.21 (m, 4H), 6.84~6.90 (m, 1H), 6.16(d, J = 4.4 Hz, 1H), 4.68~4.77 (m, 1H), 4.06 (s, 3H), 3.00 (d, J = 4.8 Hz, 3H), 2.88(s, 4H), 2.35~2.52 (m, 4H). | 701 |
| 743 | 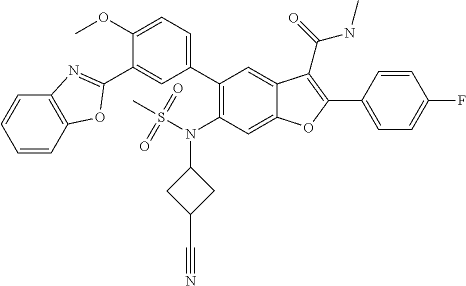 | ¹H~NMR (CDCl₃, 400 MHz) δ 8.19 (s, 1H), 7.78~7.94 (m, 3H), 7.59~7.61 (m, 1H), 7.47 (s, 1H), 7.40~7.47 (m, 2H), 7.16~7.37 (m, 5H), 6.03 (d, J = 4.4 Hz, 1H), 4.72~4.76 (d, J = 8.4 Hz, 1H), 4.08 (s, 3H), 3.00 (d, J = 4.4 Hz, 3H), 2.84~2.90 (m, 4H), 2.33~2.55 (m, 4H). | 665 |
| 744 | 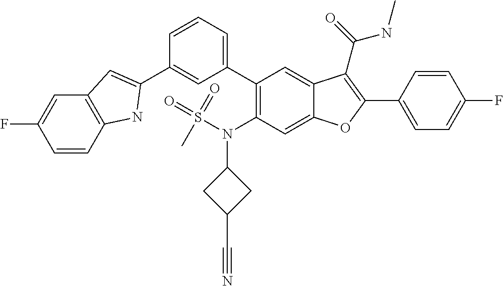 | ¹H~NMR (CDCl₃, 400 MHz) δ 9.23 (s, 1H), 7.92~8.01 (m, 4H), 7.73 (d, J = 8.0 Hz, 1H), 7.52 (d, J = 8.0 Hz, 1H), 7.21~7.43 (m, 6H), 6.89~6.95 (m, 1H), 6.80 (d, J = 1.2 Hz, 1H), 5.98 (d, J = 4.4 Hz, 1H), 4.12~4.23 (m, 1H), 3.12 (s, 3H), 3.00 (d, J = 4.8 Hz, 3H), 1.71~2.58 (m, 5H). | 651 |
| 745 | 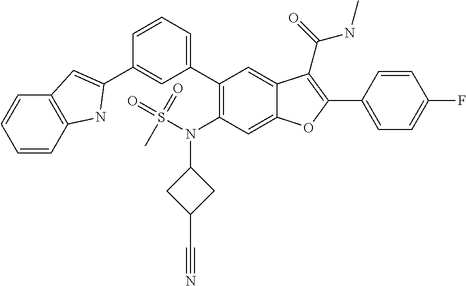 | ¹H~NMR (CDCl₃, 400 MHz) δ 9.17 (s, 1H), 7.90~7.99 (m, 4H), 7.75 (d, J = 8.0 Hz, 1H), 7.63 (d, J = 7.6 Hz, 1H),7.52 (d, J = 7.6 Hz, 1H), 7.08~7.44 (m, 7H), 6.86 (s, 1H), 6.07 (d, J = 4.4 Hz, 1H), 4.17~4.21 (m, 1H), 3.09 (s, 3H), 3.01 (d, J = 4.8 Hz, 3H), 1.79~2.61 (m, 5H). | 633 |
| 746 | 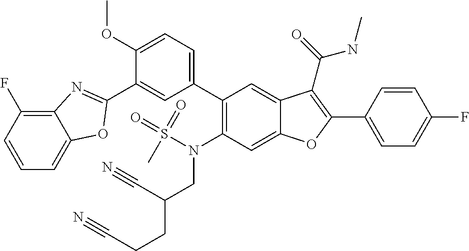 | ¹H~NMR (CDCl₃, 400 MHz) δ 7.91~7.93 (m, 1H), 7.65~7.86 (m, 5H), 7.44 (t, J = 8.4 Hz, 1H), 7.08~7.35 (m, 5H), 5.87~5.91 (m, 1H), 4.06~4.20 (m, 4H), 3.55~3.94 (m, 4H), 2.99 (d, J = 4.8 Hz, 3H), 2.66 (s, 3H), 1.75~1.87 (m, 2H). | 710 |
| 747 | 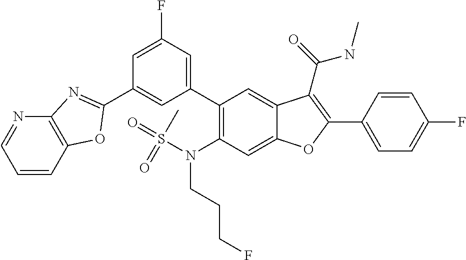 | ¹H~NMR (CDCl₃, 400 MHz) δ 8.50~8.55 (m, 1H), 8.15 (s, 1H), 7.95~7.97 (m, 1H), 7.84~7.89 (m, 4H), 7.59 (s, 1H), 7.47 (d, J = 9.2 Hz, 1H), 7.26~7.29 (m, 1H), 7.12~7.19 (m, 2H), 6.02 (d, J = 4.8 Hz, 1H), 4.00~4.23 (m, 2H), 3.47~3.49 (m, 2H), 2.93~2.97 (m, 6H), 1.67~1.75 (m, 2H). | 635 |
| 748 | 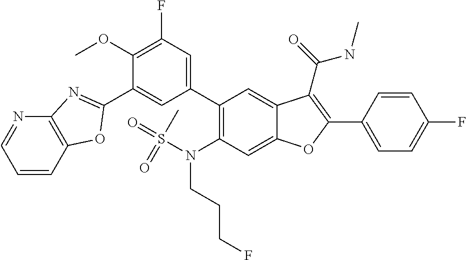 | ¹H~NMR (CDCl₃, 400 MHz) δ 8.54 (s, 1H), 8.01 (s, 1H), 7.82~7.88 (m, 4H), 7.54~7.58 (m, 2H), 7.29 (s, 1H), 7.12~7.16 (m, 2H), 5.92 (d, J = 6.4 Hz, 1H), 4.04~4.25 (m, 5H), 3.50 (s, 2H), 3.03 (s, 3H), 2.94 (s, 3H), 1.62~1.80 (m, 2H). | 665 |
| 749 | 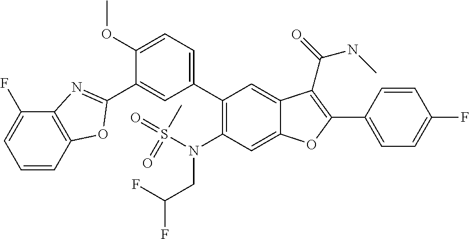 | ¹H~NMR (MeOD, 400 MHz) δ 8.23 (s, 1H), 7.84~7.96 (m, 2H), 7.81~7.83 (m, 2H), 7.69 (s, 1H), 7.38~7.41 (m, 1H), 7.35~7.37 (m, 1H), 7.30~7.32 (m, 2H), 7.22~7.26 (m, 1H), 7.11~7.16 (m, 1H), 5.74~6.03 (m, 1H), 4.04 (s, 3H), 3.84~3.93 (m, 1H), 3.41~3.59 (m, 1H), 3.16 (s, 3H), 2.93 (s, 3H). | 668 |
| 750 | 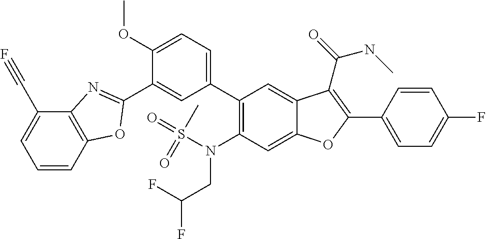 | ¹H~NMR (CDCl₃, 400 MHz) δ 8.25 (d, J = 2.6 Hz, 1H), 7.81~7.89 (m, 2H), 7.76~7.80 (m, 3H), 7.56~7.60 (m, 2H), 7.37 (d, J = 8.0 Hz, 1H), 7.11~7.19 (m, 3H), 5.91 (d, J = 4.4 Hz, 1H), 5.57~5.71 (m, 1H), 4.02 (s, 3H), 3.83~3.86 (m, 1H), 3.26~3.30 (m, 1H), 3.13 (s, 3H), 2.91 (d, J = 4.8 Hz, 3H). | 675 |
| 751 | 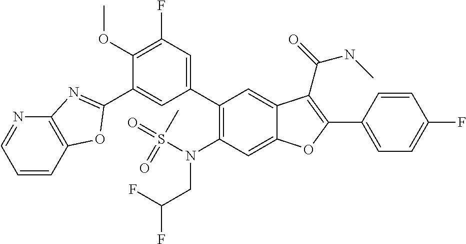 | ¹H~NMR (MeOD, 400 MHz) δ 8.54~8.55 (m, 1H), 8.15~8.20 (m, 2H), 7.96~8.00 (m, 2H), 7.89~7.91 (m, 1H), 7.68~7.77 (m, 2H), 7.48~7.50 (m, 1H), 7.24~7.28 (m, 2H), 5.77~6.04 (m, 1H), 4.31 (s, 1H), 4.14 (s, 3H), 3.80 (s, 5H), 2.92 (s, 3H) | 669 |
| 752 | 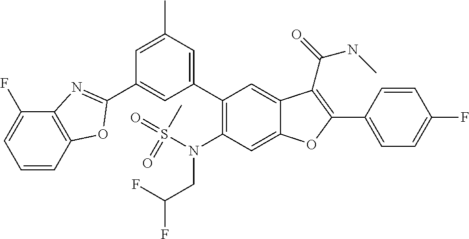 | ¹H~NMR (CDCl₃, 400 MHz) δ 8.09 (s, 2H), 7.85~7.88 (m, 2H), 7.79 (s, 1H), 7.57(s, 1H), 7.54 (s, 1H), 7.23~7.34 (m, 2H), 7.12 (t, J = 8.0 Hz, 2H), 7.01 (t, J = 8.4 Hz, 1H), 6.99 (br s, 1H), 5.22~5.83 (m, 1H), 3.78~3.81 (m, 0.5H), 3.25~3.29 (m, 0.5H), 3.03 (s, 3H), 2.91 (d, J = 4.0 Hz, 3 H), 2.44 (s, 3H). | 652 |
| 753 | 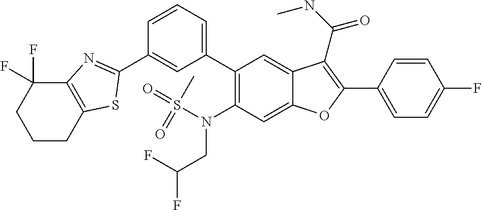 | ¹H-NMR (300 MHz, CD₃CN): δ 8.12~8.11 (m, 1H), 8.05~8.00 (m, 3H), 7.78 (s, 2H), 7.69~7.66 (m, 1H), 7.63~7.56 (m, 1H), 7.29 (t, J = 8.7 Hz, 2H), 6.86~6.83 (m, 1H), 5.87~5.84 (m, 1H), 3.83 (br, 1H), 3.45 (br, 1H), 3.11 (s, 3H), 2.98~2.92 (m, 2H), 2.86 (d, 3H), 2.20~1.94 (m, 3H), 1.77~1.61 (m, 1H). | 676 |
| 754 | 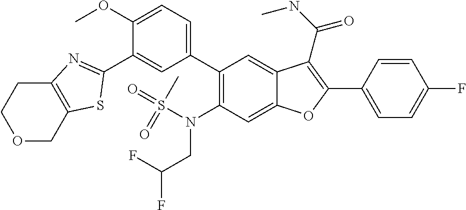 | ¹H-NMR (CDCl₃, 300 MHz): δ 8.42 (s, 1H), 8.00~8.04 (m, 2H), 7.91~7.94 (m, 1H), 7.81 (s, 1H), 7.56 (s, 1H), 7.18~7.21 (m, 3H), 6.80 (s, 1H), 5.53~5.89 (m, 1H), 4.89 (s, 2H), 4.06~4.11 (m, 3H), 3.84~3.92 (m, 4H), 3.19 (s, 3H), 3.00~3.01 (m, 5H). | 672 |
| 755 | 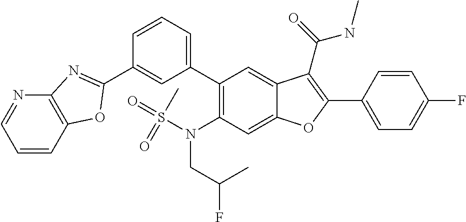 | ¹H~NMR (CDCl₃, 400 MHz) δ 8.42 (s, 1H), 8.34 (d, J = 3.6 Hz, 1H), 7.94 (d, J = 7.2 Hz, 1H), 7.59~7.88 (m, 7H), 7.34~7.37 (m, 1H), 7.19 (t, J = 8.4 Hz, 2H), 6.10(br s, 1H), 4.43~4.93 (m, 1H), 2.70~3.79 (m, 8H), 1.08~1.16 (m, 3H). | 617 |
| 756 | 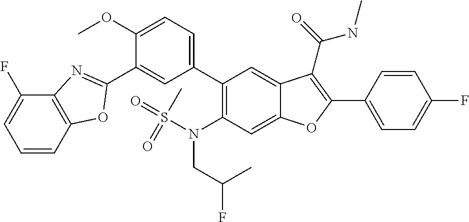 | ¹H~NMR (CDCl₃, 400 MHz) δ 8.18 (s, 1H), 7.90~7.96 (m, 3H), 7.82 (s, 1H), 7.52 (s, 1H), 7.35 (d, J = 8.0 Hz, 1H), 7.23~7.27 (m, 1H), 7.13~7.17 (m, 3H), 7.01 (t, J = 8.8 Hz, 1H), 5.82 (s, 1H), 4.34~4.50 (m, 1H), 4.02 (s, 3H), 3.67~3.76 (m, 1H), 3.19 (s, 2H), 2.94 (t, J = 4.8 Hz, 4H), 2.66~2.80 (m, 1H), 1.04~1.18 (m, 3H). | 664 |
| 757 | 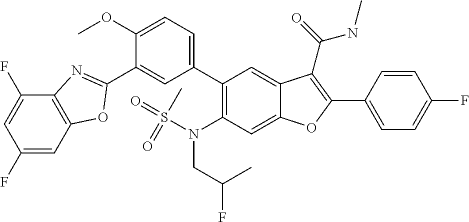 | ¹H~NMR (CDCl₃, 400 MHz) δ 8.15 (s, 1H), 7.89~7.95 (m, 3H), 7.82 (s, 1H), 7.52 (s, 1H), 7.11~7.17 (m, 4H), 6.82 (t, J = 8.0 Hz, 1H), 5.81 (d, J = 5.2 Hz, 1H), 4.41~4.52 (m, 1H), 4.02 (s, 3H), 3.57~3.77 (m, 1H), 3.19 (s, 2H), 2.94 (d, J = 4.8 Hz, 4H), 2.66~2.79 (m, 1H), 1.04~1.19 (m, 3H). | 682 |
| 758 | 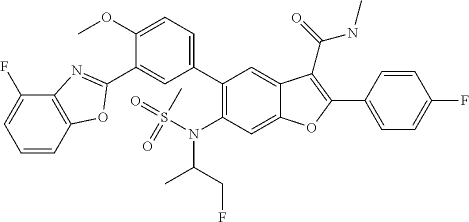 | ¹H~NMR (CDCl₃, 400 MHz)δ 8.20~7.95 (m, 2H), 7.94~7.88 (m, 3H), 7.80~7.77 (m, 1H), 7.55 (d, J = 9.6 Hz, 1H), 7.36~7.25 (m, 1H), 7.24~7.14 (m, 3H), 7.13~6.97 (m, 1H), 5.96 (d, J = 2.2 Hz, 1H), 4.36~4.09 (m, 1H), 4.06~3.94 (m, 5H), 3.16~2.98 (m, 3H), 2.97~2.94 (m, 3H), 1.03~1.01 (m, 1H), 0.41 (d, J = 3.0 Hz, 2H). | 664 |
| 759 | 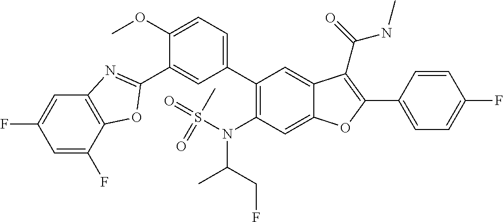 | ¹H~NMR (CDCl₃, 400 MHz) δ 7.81~8.17 (m, 5H), 7.53~7.58 (m, 1H), 7.13~7.26 (m, 4H), 6.83 (t, J = 7.6 Hz, 1H), 5.82 (s, 1H), 4.25~4.36 (m, 1H), 3.71~4.15 (m, 5H), 3.16 (s, 3H), 2.93 (d, J = 4.4 Hz, 3H), 1.04 (d, J = 5.6 Hz, 1H), 0.42 (d, J = 6.0 Hz, 2H). | 682 |
| 760 | 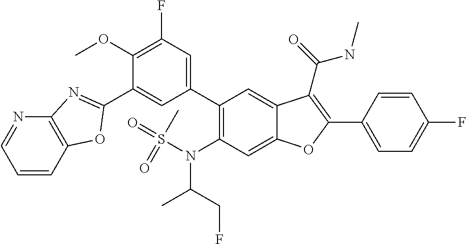 | ¹H~NMR (CDCl₃, 400 MHz) δ 8.55 (s, 1H), 7.82~8.08 (m, 5H), 7.55~7.62 (m, 2H), 7.15~7.37 (m, 3H), 5.95 (d, J = 5.6 Hz, 1H), 3.89~4.39 (m, 6H), 3.17 (s, 2H), 2.95 (s, 3H), 1.00~1.07 (m, 1H), 0.42~0.48 (m, 2H). | 665 |
| 761 | 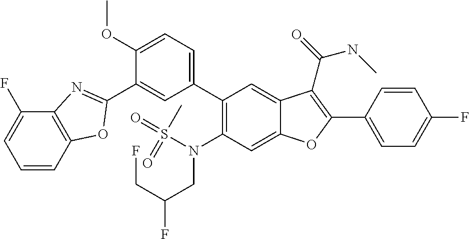 | ¹H~NMR (MeOD, 400 MHz) δ 8.28 (s, 1H), 7.96~7.98 (m, 2H), 7.81~7.90 (m, 2H), 7.71 (d, J = 14.0 Hz, 1H), 7.49 (d, J = 8.0 Hz, 1H), 7.37~7.42 (m, 1H), 7.33 (d, J = 8.0 Hz, 1H), 7.26 (t, J = 8.0 Hz, 2H), 7.15 (t, J = 9.0 Hz, 1H), 4.36~4.47 (m, 2H), 4.05 (s, 3H), 3.87~3.92 (m, 1H), 3.47~3.59 (m, 2H), 3.22 (s, 3H), 2.93 (s, 3H). | 682 |
| 762 | 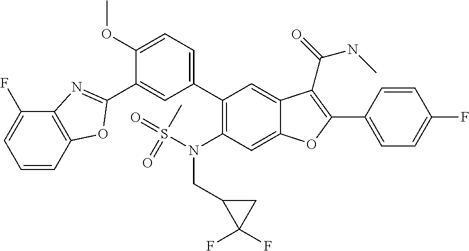 | ¹H~NMR (CDCl₃, 400 MHz) 8.17~8.21 (m, 1H), 7.90 (t, J = 3.2 Hz, 2H), 7.81 (d, J = 8.8 Hz, 1H), 7.61~7.64 (m, 2H), 7.52 (s, 1H), 7.34~7.36 (m, 1H), 7.21~7.27 (m, 3H), 7.01~7.03 (m, 1H), 5.85~5.87 (m, 1H), 4.10 (s, 3H), 3.02~3.17 (m, 2H), 2.80~2.99 (m, 6H), 0.77~1.40 (m, 3H). | 694 |
| 763 | 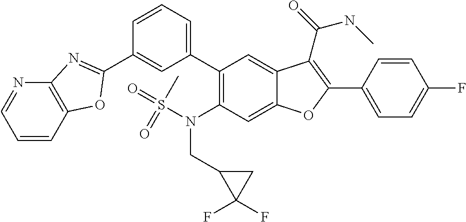 | H~NMR (CDCl₃, 400 MHz) δ 8.28~8.33 (m, 3H), 7.81~7.92 (m, 4H), 7.55~7.64 (m, 3H), 7.25~7.27 (m, 1H), 7.13~7.24 (m, 2H), 5.88 (br s, 1H), 2.98~3.76 (m, 3H), 2.93 (d, J = 8.0 Hz, 3H), 2.79 (s, 2H), 097~0.99 (m, 1H), 0.76~0.82 (m, 2H). | 647 |
| 764 | 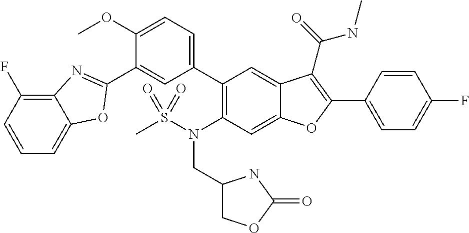 | ¹H-NMR (CDCl₃) 400 MHz) δ 8.15 (s, 1H), 7.72~7.83 (m, 4H), 7.57 (s, 1H), 7.35~7.36 (m, 1H), 7.24 (s, 1H), 7.11 (s, 3H), 6.98~7.02 (m, 1H), 6.29 (s, 0.5H), 6.13 (s, 0.5H), 5.95 (s, 0.5H), 4.91 (s, 0.4H), 4.23 (s, 0.5H), 4.00 (s, 3H), 3.63~3.76 (m, 2.6H), 3.40 (d, J = 14.4 Hz, 2H), 3.08 (s, 3H), 2.95 (s, 3H). | 703 |
| 765 | 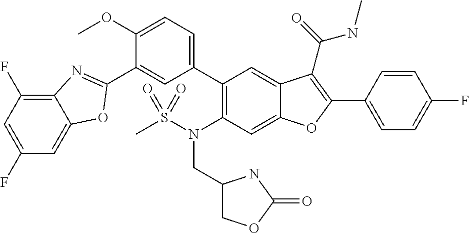 | ¹H-NMR (MeOD, 400 MHz) δ 8.24 (s, 1H), 7.83~7.96 (m, 4H), 7.71 (s, 1H), 7.30~7.35 (m, 2H), 7.22~7.26 (m, 2H), 7.04 (t, J = 9.4 Hz, 1H), 4.19~4.24 (m, 0.3H), 4.04 (s, 3H), 3.81~3.86 (m, 1.3H), 3.46~3.56 (m, 3.4H), 3.18 (s, 3H), 2.93 (s, 3H). | 721 |
| 766 | 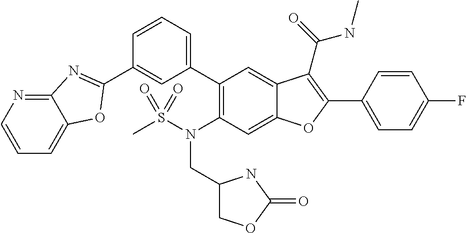 | ¹H-NMR (MeOD, 400 MHz) δ 8.53~8.54 (m, 2H), 8.29~8.38 (m, 1H), 8.15 (d, J = 8.0 Hz, 1H), 7.90~8.00 (m, 4H), 7.71~7.75 (m, 2H), 7.46~7.49 (m, 1H), 7.27 (t, J = 8.4 Hz, 2H), 3.39~4.24 (m, 5H), 3.17 (s, 3H), 2.92 (s, 3H). | 656 |
| 767 | 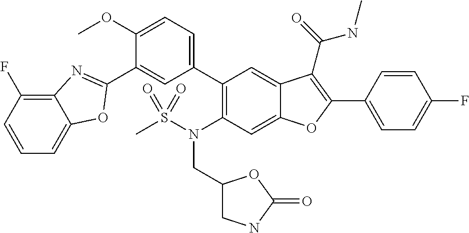 | ¹H-NMR (CDCl₃, 400 MHz) δ 8.24 (s, 1H), 8.05 (d, J = 8.4 Hz, 1H), 7.94~7.98 (m, 2H), 7.91 (s, 1H), 7.63~7.70 (m, 1H), 7.43 (d, J = 8.0 Hz, 1H), 7.27~7.34 (m, 1H), 7.20~7.24 (m, 3H), 7.08 (t, J = 8.8 Hz, 1H), 5.93 (s, 1H), 4.42 (s, 1H), 4.37~4.46 (m, 1H), 4.08 (s, 3H), 3.83~3.89 (m, 1H), 3.35 (s, 3H), 2.99~3.07 (m, 5H). | 703 |
| 768 | 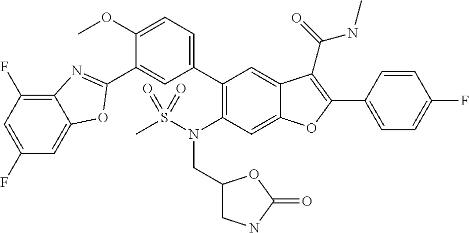 | ¹H-NMR (CDCl₃, 400 MHz) δ 8.20 (d, J = 2.0 Hz, 1H), 7.90~8.03 (m, 4H), 7.70 (s, 1H), 7.18~7.25 (m, 4H), 6.81~6.92 (m, 2H), 5.99 (s, 1H), 5.04 (s, 1H), 4.38~4.41 (m, 1H), 4.09 (s, 3H), 3.81~3.88 (m, 1H), 3.33 (s, 3H), 2.98~3.05 (m, 5H). | 721 |
| 769 | 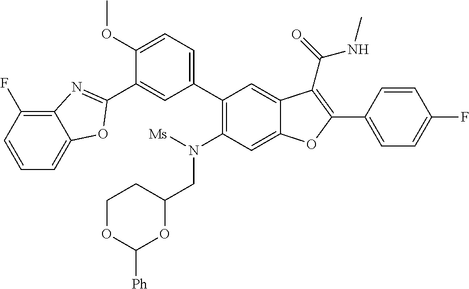 | ¹H-NMR (CDCl₃, 400 MHz) δ 7.79~8.00 (m, 5H), 7.05~7.47 (m, 12H), 6.07 (d, J = 4.4 Hz, 1H), 5.37 (s, 1H), 3.60~4.12 (m, 7H), 2.88~3.01 (m, 7H), 1.16~1.34 (m, 2H). | 780 |
| 770 | 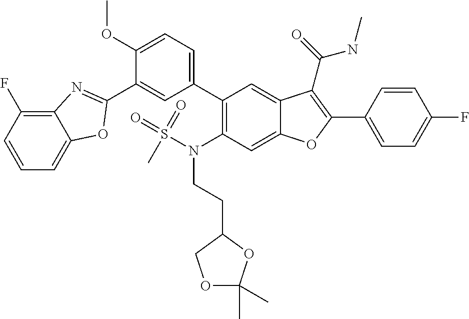 | ¹H-NMR (MeOD, 400 MHz) δ 8.18~ 8.21 (m, 1H), 7.88~7.91 (m, 2H), 7.76 (s, 2H), 7.67 (s, 1H), 7.45 (d, J = 8.0 Hz, 1H), 7.32~7.37 (m, 1H), 7.26 (d, J= 8.8 Hz, 1H), 7.19~7.22 (m, 2H), 7.08~7.17 (m, 1H), 4.01 (s, 3H), 3.83~3.88 (m, 1H), 3.34~3.66 (m, 3H), 3.19~3.33 (m, 1H), 3.09 (d, J = 21.6 Hz, 3H), 2.93 (s, 3H), 1.44~1.56 (m, 2H), 1.08~1.19 (m, 6H). | 732 |
| 771 | 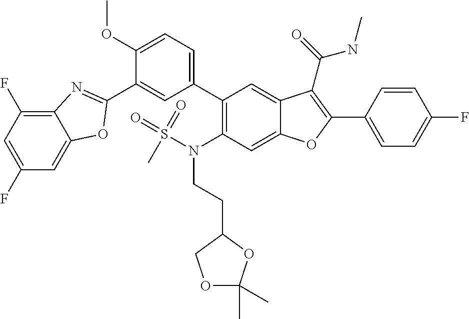 | ¹H-NMR (MeOD, 400 MHz) δ 8.17~ 8.19 (m, 1H), 7.89~7.92 (m, 2H), 7.76 (d, J = 7.2 Hz, 2H), 7.67 (s, 1H), 7.33 (d, J = 7.2 Hz, 1H), 7.18~ 7.28 (m, 3H), 7.02 (t, J = 9.6 Hz, 1H), 4.01 (s, 3H), 3.83~3.90 (m, 1H), 3.42~3.65 (m, 3H), 3.20~3.37 (m, 1H), 3.09 (d, J = 20.0 Hz, 3H), 2.93 (s, 3H), 1.45~1.57 (m, 2H), 1.09~1.20 (m, 6H). | 750 |
| 772 | 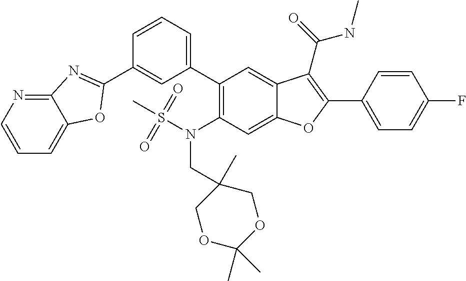 | ¹H-NMR (CDCl₃, 400 MHz) δ 8.57~8.58 (m, 1H), 8.53 (s, 1H), 8.36~8.38 (d, J = 7.6 Hz, 4H), 7.88~7.97 (m, 5H), 7.84 (s, 1H), 7.72 (s, 1H), 7.62~7.69 (m, 1H), 7.30~7.33 (m, 1H), 7.18~7.22 (m, 2H), 3.58~3.70 (m, 2H), 3.37~3.47 (m, 3H), 3.29 (s, 3H), 3.11~3.17 (m, 1H), 2.98 (s, 3H), 1.29 (s, 3H), 1.27 (s, 3H). | 699 |
| 773 | 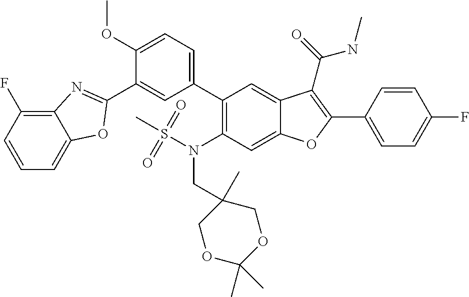 | ¹H-NMR (CDCl₃, 400 MHz) δ 8.35 (s, 1H), 7.89~7.98 (m, 3H), 7.82 (s, 1H), 7.70 (s, 1H), 7.40~7.42 (m, 1H), 7.29~7.33 (m, 1H), 7.16~7.24 (m, 3H), 7.05~7.09 (m, 1H), 5.87~5.88 (m, 1H), 4.10 (s, 3H), 3.65 (s, 2H), 3.43 (s, 2H), 3.33 (s, 3H), 3.16~3.21 (m, 1H), 2.97~3.00 (m, 4H), 1.29~1.31 (m, 6H), 0.31 (s, 3H), 2.62~2.65 (m, 1H), 1.70~1.80 (m, 1H), 1.35~1.45 (m, 1H). | 746 |
| 774 | 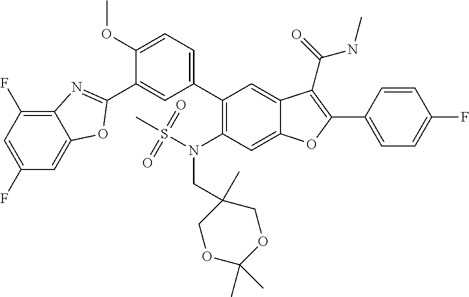 | ¹H-NMR (CDCl₃, 400 MHz) δ 8.32 (s, 1H), 7.88~7.96 (m, 3H), 7.81 (s, 1H), 7.69 (s, 1H), 7.16~7.22 (m, 4H), 6.85~6.91 (m, 1H), 5.90~5.91 (m, 1H), 4.09 (s, 3H), 3.63~3.65 (m, 2H), 3.43~3.48 (m, 3H), 3.33 (s, 3H), 3.17~3.20 (m, 2H), 2.96~2.99 (m, 3H), 1.29~1.31 (m, 6H), 0.30(s, 3H). | 764 |

### Example 775

### 2-(4-fluorophenyl)-6-(N-(2-hydroxy-2-methylpropyl)methylsulfonamido)-N-methyl-5-(3-(oxazolo[4,5-b]pyridin-2-yl)phenyl)benzofuran-3-carboxamide

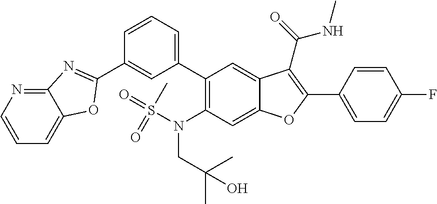

### Step 1—Synthesis of 5-bromo-2-(4-fluorophenyl)-6-(N-(2-hydroxypropyl)methylsulfonamido)-N-methylbenzofuran-3-carboxamide

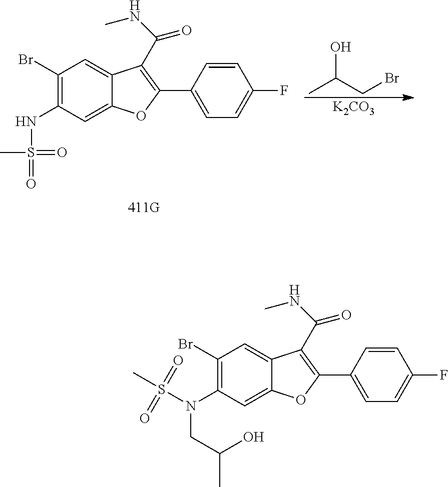

To a solution of Compound 411G (8 g, 18.1 mmol), K₂CO₃ (7.5 g, 54.3 mmol) and KI (1.5 g, 9.05 mmol) in DMF (150 mL) at 15° C. was added 1-bromopropan-2-ol (5.03 g, 36.2 mmol, 4.5 mL) dropwise. The reaction was heated to 110° C. and allowed to stir at this temperature for 8 hours. The reaction mixture was diluted with water and the resulting solution extracted with EtOAc (500 mL×5). The combined organic extracts were washed with brine, dried over Na₂SO₄, filtered and concentrated in vacuo, and the residue obtained was purified using column chromatography (dichloromethane/EtOAc=20:1 to 15:1) to provide 5-bromo-2-(4-fluorophenyl)-6-(N-(2-hydroxypropyl)methylsulfonamido)-N-methylbenzofuran-3-carboxamide (5 g, 55%) as yellow solid. ¹H-NMR (CDCl₃, 400 MHz) δ 8.11 (d, J=4.8 Hz, 1H), 7.81˜7.84 (m, 2H), 7.66˜7.71 (m, 1H), 7.15 (t, J=8.4 Hz, 2H), 5.75 (d, J=3.6 Hz, 1H), 3.60˜3.92 (m, 2H), 3.46˜3.58 (m, 1H), 3.09 (s, 3H), 2.93 (d, J=4.8 Hz, 3H), 1.09˜1.13 (m, 3H).

MS (M+H)⁺: 499.

### Step 2—Synthesis of 5-bromo-2-(4-fluorophenyl)-N-methyl-64N-(2-oxopropyl)methylsulfonamido)benzofuran-3-carboxamide

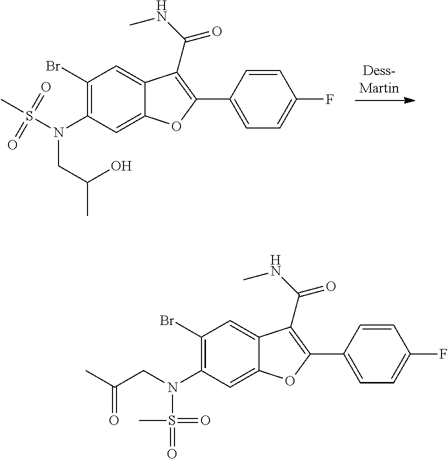

To a 0° C. solution of 5-bromo-2-(4-fluorophenyl)-6-(N-(2-hydroxypropyl)methylsulfonamido)-N-methylbenzofuran-3-carboxamide (1.00 g, 2.00 mmol) in dichloromethane (20 mL) was added DMP (1.19 g, 2.81 mmol) portionwise. The reaction was allowed to stir at 20° C. for 6 hours, then the reaction mixture was diluted with NaHCO₃ and basified to pH 8, then extracted with dichloromethane (500 mL×3). The combined organic extracts were washed with Na₂SO₃, brine and dried over Na₂SO₄, filtered and concentrated in vacuo to provide 5-bromo-2-(4-fluorophenyl)-N-methyl-6-(N-(2-oxopropyl)methylsulfonamido)benzofuran-3-carboxamide (908 mg, 91%). ¹H-NMR (CDCl₃, 400 MHz) δ 8.09 (s, 1H), 7.98 (s, 1H), 7.81˜7.85 (m, 2H), 7.15 (t, J=8.4 Hz, 2H), 5.76 (d, J=3.68 Hz, 1H), 4.86˜4.89 (m, 1H), 4.10˜4.32 (m, 1H), 3.09 (s, 3H), 2.93 (d, J=4.8 Hz, 3H), 2.08 (s, 3H). MS (M+H)⁺: 497.

### Step 3—Synthesis of 5-bromo-2-(4-fluorophenyl)-6-(N-(2-hydroxy-2-methylpropyl)methylsulfonamido)-N-methylbenzofuran-3-carboxamide

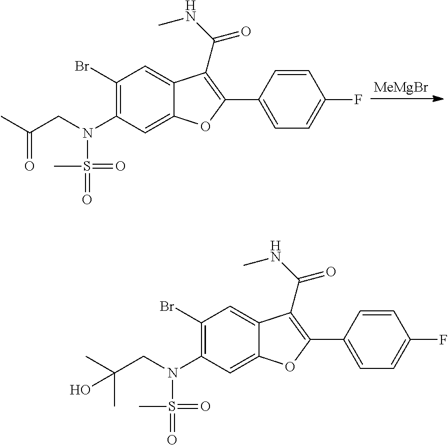

To a 0° C. solution of 5-bromo-2-(4-fluorophenyl)-N-methyl-6-(N-(2-oxopropyl)methylsulfonamido)benzofuran-3-carboxamide (1.00 g, 2.01 mmol) in anhydrous THF (20 mL) was added MeMgBr (3 M, 1 mL) and the reaction was allowed to stir at 20° C. for 3 hours. The reaction mixture was then quenched with aqueous NH₄Cl, then extracted with EtOAc (100 mL×3). The combined organic extracts were washed with brine, dried over Na₂SO₄, filtered and concentrated in vacuo to provide 5-bromo-2-(4-fluorophenyl)-6-(N-(2-hydroxy-2-methylpropyl)methylsulfonamido)-N-methylbenzofuran-3-carboxamide (910 mg, 91%). ¹H-NMR (CDCl₃, 400 MHz) δ 7.77 (d, J=3.2 Hz, 1H), 7.67˜7.71 (m, 2H), 7.51 (s, 1H), 7.26 (d, J=4.4 Hz, 1H), 6.91 (t, J=8.4 Hz, 2H), 3.57˜3.61 (m, 1H), 3.42˜3.46 (m, 1H), 2.85 (s, 3H), 2.70 (d, J=4.8 Hz, 3H), 0.97 (s, 3H), 0.93 (s, 3H). MS (M+H)⁺: 513.

### Step 4—Synthesis of 2-(4-fluorophenyl)-6-(N-(2-hydroxy-2-methylpropyl)methylsulfonamido)-N-methyl-5-(3-(oxazolo[4,5-b]pyridin-2-yl)phenyl)benzofuran-3-carboxamide

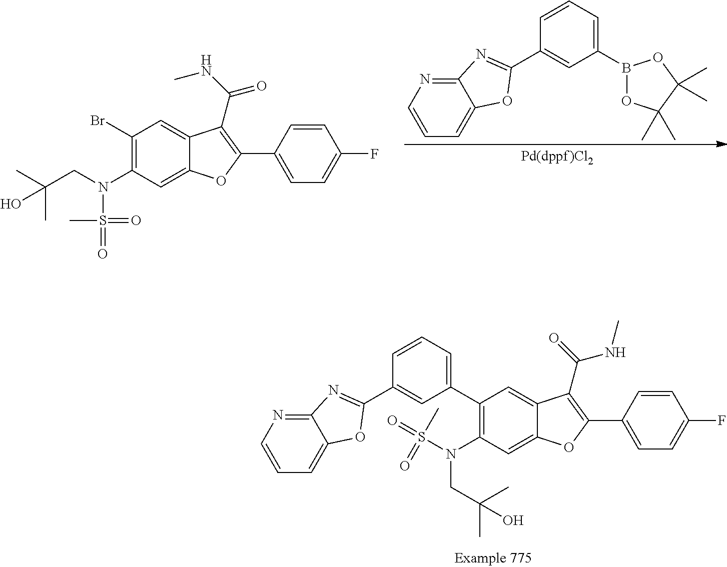

### 5-bromo-2-(4-fluorophenyl)-6-(N-(2-hydroxy-2-methylpropyl)methylsulfonamido)-N-methylbenzofuran-3-carboxamide was converted to the title compound (800 mg, 65.6%) using the method described in Example 411, Step 12. ¹H-NMR (CDCl₃, 400 MHz) δ 8.52 (t, J=1.6 Hz, 1H), 8.48 (s, 1H), 8.30 (d, J=8.0 Hz, 1H), 7.88˜7.91 (m, 3H), 7.84 (t, J=6.8 Hz, 1H), 7.82 (s, 1H), 7.79 (s, 1H), 7.59˜7.65 (m, 1H), 7.24˜7.27 (m, 1H), 7.15 (t, J=8.4 Hz, 2H), 5.83 (d, J=4.8 Hz, 1H), 3.45 (t, J=12.8 Hz, 1H), 3.20 (s, 3H), 3.05 (d, J=15.2 Hz, 1H), 2.93 (d, J=4.8 Hz, 3H), 1.68˜1.72 (m, 1H), 0.95 (s, 6H). MS (M+H)⁺: 629.

The following compounds of the present invention were made using the method described above and using the appropriate reactants and/or reagents.

| | | | MS |
|---|---|---|---|
| Example | Structure | NMR | (M + H)⁺ |
| 776 | 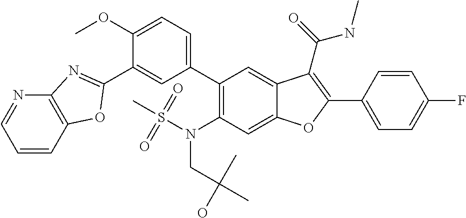 | ¹H-NMR (CDCl₃, 400 MHz) δ 8.52 (s, 1H), 7.85 (s, 1H), 7.87~7.90 (m, 3H), 7.83 (d, J = 6.8 Hz, 1H), 7.76 (s, 1H), 7.62 (s, 1H), 7.21~7.25 (m, 1H), 7.11~7.19 (m, 3H), 5.96 (d, J = 4.8 Hz, 1H), 4.02 (s, 3H), 3.49 (d, J = 15.2 Hz, 1H), 3.23 (s, 3H), 3.06 (d, J = 15.2 Hz, 1H), 2.93 (d, J = 4.8 Hz, 3H), 0.95 (d, J = 2.8 Hz, 6H). | 659 |
| 777 | 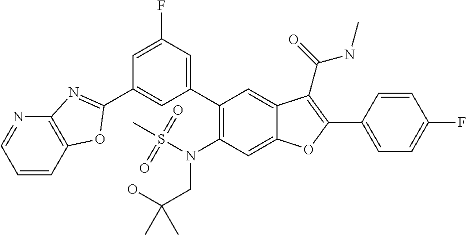 | ¹H-NMR (CDCl₃, 400 MHz) δ 8.26 (s, 1H), 7.87~7.92 (m, 3H), 7.77 (s, 1H), 7.64 (s, 1H), 7.54 (d, J = 8.0 Hz, 1H), 7.22~7.25 (m, 1H), 7.12~7.17 (m, 3H), 7.04 (t, J = 8.8 Hz, 1H), 5.80 (d, J = 4.8 Hz, 1H), 4.05 (s, 3H), 3.49 (d, J = 15.2 Hz, 1H), 3.24 (s, 3H), 3.06 (d, J = 15.2 Hz, 1H), 2.93 (d, J = 4.8 Hz, 3H), 0.96 (d, J = 3.6 Hz, 6H). | 647 |
| 778 | 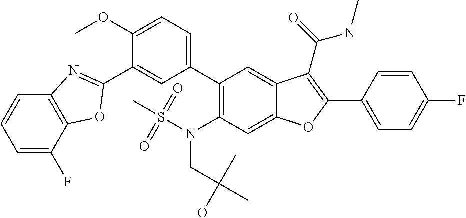 | ¹H-NMR (CDCl₃, 400 MHz) δ 8.33 (s, 1H), 7.92~7.95 (m, 3H), 7.81 (s, 1H),7.67 (s, 1H), 7.40 (d, J = 8.0 Hz, 1H), 7.27~7.32 (m, 1H), 7.18 (t, J = 8.0 Hz, 3H), 7.06 (t, J = 8.8 Hz, 1H), 5.96 (s, 1H), 4.07 (s, 3H), 3.54 (d, J = 15.2 Hz, 1H), 3.29 (s, 3H), 3.10 (d, J = 15.2 Hz, 1H), 2.99 (d, J = 4.0 Hz, 3H), 1.00 (s, 6H). | 676 |
| 779 | 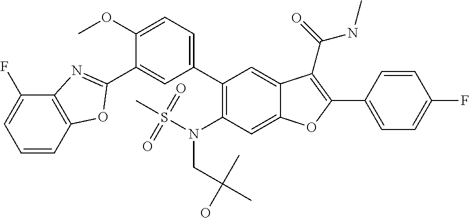 | ¹H-NMR (CDCl₃, 400 MHz) δ 8.33 (s, 1H), 7.92~7.95 (m, 3H), 7.81 (s, 1H), 7.67 (s, 1H), 7.40 (d, J = 8.0 Hz, 1H), 7.27~7.32 (m, 1H), 7.18 (t, J = 8.0 Hz, 3H), 7.06 (t, J = 8.8 Hz, 1H), 5.96 (s, 1H), 4.07 (s, 3H), 3.54 (d, J = 15.2 Hz, 1H), 3.29 (s, 3H), 3.10 (d, J = 15.2 Hz, 1H), 2.99 (d, J = 4.0 Hz, 3H), 1.00(s, 6H). | 676 |
| 780 | 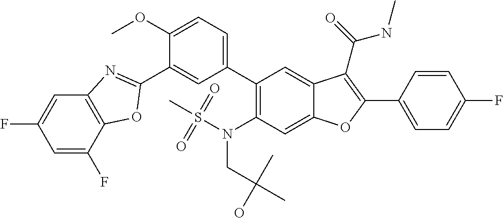 | ¹H-NMR (CDCl₃, 400 MHz) δ 8.31 (s, 1H), 7.92~7.97 (m, 3H), 7.83 (s, 1H), 7.68 (s, 1H), 7.31 (d, J = 8.8 Hz, 2H), 7.19 (d, J = 8.4 Hz, 2H), 6.87~6.92 (m, 1H), 5.84 (d, J = 2.4 Hz, 1H), 4.09 (s, 3H), 3.54 (d, J = 14.8 Hz, 1H), 3.29 (s, 3H), 3.11 (d, J = 15.2 Hz, 1H), 2.98 (d, J = 4.8 Hz, 3H), 1.01 (d, J = 2.8 Hz, 6H). | 694 |

### Example 781

### 6-(N-(2,4-dihydroxybutyl)methylsulfonamido)-5-(3-(4-fluorobenzo[d]oxazol-2-yl)-4-methoxyphenyl)-2-(4-fluorophenyl)-N-methylbenzofuran-3-carboxamide

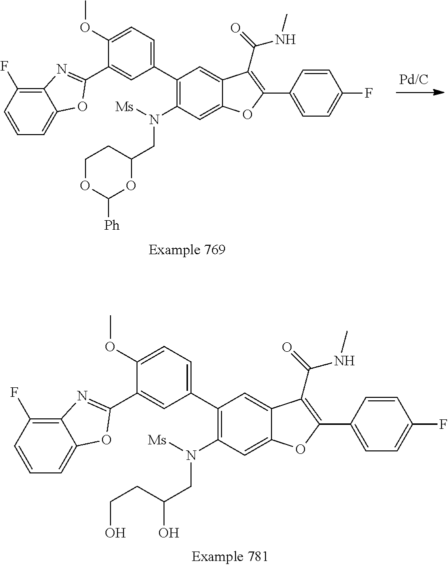

A mixture of the compound of Example 769 (50 mg, 0.06 mmol) and Pd/C (5 mg) in MeOH (5 mL) was placed under hydrogen atmosphere (50 psi) and allowed to stir at room temperature for 5 hours. The reaction mixture was filtered and concentrated in vacuo, and the residue obtained was purified using prep-HPLC to provide the title compound (30 mg, 68%). ¹H-NMR (CDCl₃, 400 MHz) δ 8.12˜8.17 (m, 1H), 7.72˜7.87 (m, 4H), 7.62 (d, J=3.6 Hz, 1H), 7.00˜7.43 (m, 6H), 6.38˜6.57 (m, 1H), 4.00 (s, 3H), 3.51˜3.72 (m, 4H), 2.81˜3.14 (m, 7H), 1.31˜1.53 (m, 2H). MS (M+H)⁺: 692.

The following compounds of the present invention were made using the method described above and using the appropriate reactants and/or reagents.

| | | | MS |
|---|---|---|---|
| Example | Structure | NMR | (M + H)⁺ |
| 782 | 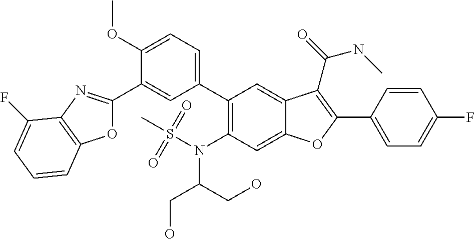 | ¹H-NMR (CDCl₃, 400 MHz) δ 8.07~8.16 (m, 1H), 7.63~7.80 (m, 4H), 7.54 (s, 1H), 6.95~7.38 (m, 6H), 6.18~6.29 (m, 1H), 3.94 (d, J = 8.4 Hz, 3H), 3.39~3.76 (m, 4H), 2.66~3.20 (m, 7H). | 678 |
| 783 | 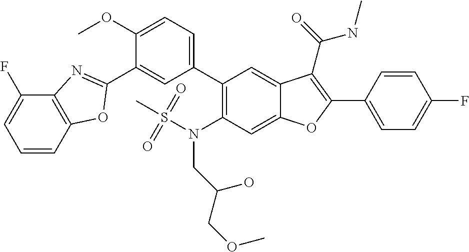 | ¹H-NMR (CDCl₃, 400 MHz) δ 8.12~8.17 (m, 1H), 7.72~7.87 (m, 4H), 7.62 (d, J = 3.6 Hz, 1H), 7.00~7.43 (m, 6H), 6.38-6.57 (m, 1H), 4.00 (s, 3H), 3.51~3.72 (m, 4H), 2.81~3.14 (m, 7H), 1.31~1.53 (m, 2H). | 692 |
| 784 | 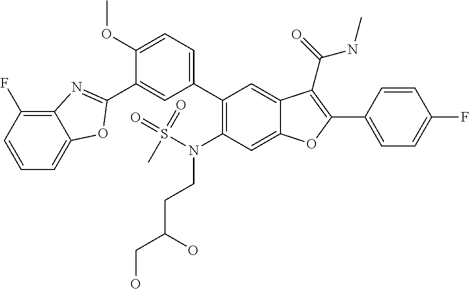 | ¹H-NMR (MeOD, 400 MHz) δ 8.25~8.27 (m, 1H), 7.95-7.99 (m, 2H), 7.81~7.84 (m, 2H), 7.70 (s, 1H), 7.51 (d, J = 8.8 Hz, 1H), 7.32~7.41 (m, 2H), 7.23~7.28 (m, 2H), 7.12~7.14 (m, 1H), 4.05 (s, 3H), 3.46~3.41 (m, 3H), 3.21 (s, 1H), 3.09 (d, J = 20.0 Hz, 3H), 2.93 (s, 3H), 1.43~1.65 (m, 3H) | 692 |
| 785 | 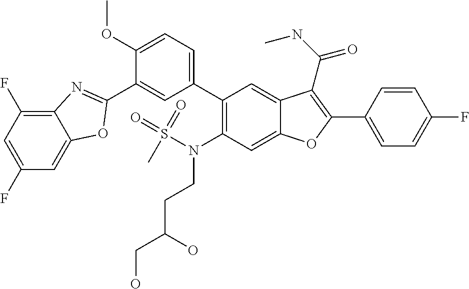 | ¹H-NMR (MeOD, 400 MHz) δ 8.24~8.25 (m, 1H), 7.96~7.99 (m, 2H), 7.83 (s, 2H), 7.69 (s, 1H), 7.38 (d, J = 6.8 Hz, 1H), 7.33 (d, J = 8.8 Hz, 1 H), 7.24~7.28 (m, 2H), 7.04~7.07 (m, 1H), 4.05 (s, 3H), 3.46 (s, 3H), 3.21 (s, 1H), 3.09 (d, J = 19.6 Hz, 3H), 2.93 (s, 3H), 1.43~1.63 (m, 3H). | 710 |
| 786 | 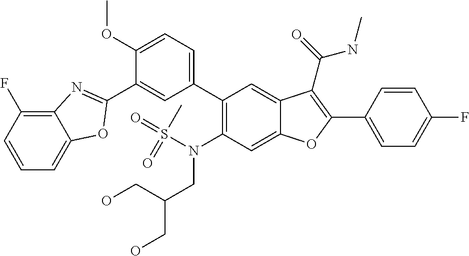 | ¹H-NMR (CDCl₃, 400 MHz) δ 8.10 (d, J = 2.2 Hz, 1H), 7.90~7.94 (m, 2H), 7.75~7.77 (m, 2H), 7.60 (s, 1H), 7.41 (d, J = 8.0 Hz, 1H), 7.27~7.32 (m, 1H), 7.15 (t, J = 8.4 Hz, 3H), 7.06 (t, J = 8.8 Hz, 1H), 6.16 (d, J = 4.6 Hz, 1H), 4.05 (s, 3H), 3.65 (d, J = 4.0 Hz, 2H), 3.51~3.60 (m, 2H), 3.45~3.49 (m, 2H), 3.21-3.25 (m, 1H), 3.01 (s, 3H), 2.97 (d, J = 4.8 Hz, 3H), 2.05~2.15 (m, 2H). | 692 |
| 787 | 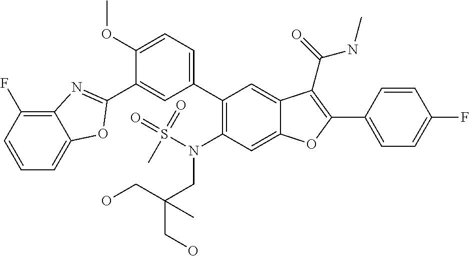 | ¹H-NMR (CDCl₃, 400 MHz) δ 8.27 (s, 1H), 7.92~7.97 (m, 3H), 7.83 (s, 1H), 7.69 (s, 1H), 7.41~7.43 (m, 1H), 7.28~7.33 (m, 1H), 7.18~7.23 (m, 3H), 7.05~7.09 (m, 1H), 5.94~5.96 (m, 1H), 4.10~4.13 (m, 3H), 3.58~3.69 (m, 2H), 3.40~3.49 (q, 2H), 3.21~3.27 (m, 4H), 3.13~3.16 (m, 1H), 3.00 (d, J = 8.0 Hz, 3H), 1.87 (s, 2H), 0.36 (s, 3H). | 706 |
| 788 | 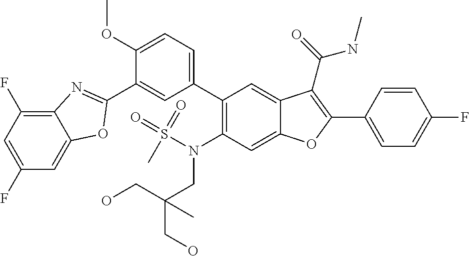 | ¹H-NMR (CDCl₃, 400 MHz) δ 8.19 (s, 1H), 7.83~7.88 (m, 3H), 7.76 (s, 1H), 7.63 (s, 1H), 7.10~7.16 (m, 4H), 6.82~6.87 (m, 1H), 6.45 (s, 1H), 4.04 (s, 3H), 3.55 (s, 2H), 3.33~3.34 (m, 2H), 3.15~3.18 (m, 5H), 3.04~3.07 (m, 2H), 2.97~2.98 (m, 3H), 0.30 (s, 3H). | 724 |

### Example 789

### 64N-(2-fluoro-2-methylpropyl)methylsulfonamido)-5-(3-(4-fluorobenzo[d]oxazol-2-yl)-4-methoxyphenyl)-2-(4-fluorophenyl)-N-methylbenzofuran-3-carboxamide

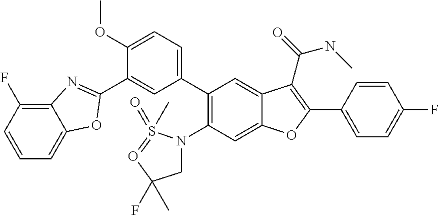

### Step 1—Synthesis of 5-bromo-6-(N-(2-fluoro-2-methylpropyl)methylsulfonamido)-2-(4-fluorophenyl)-N-methylbenzofuran-3-carboxamide

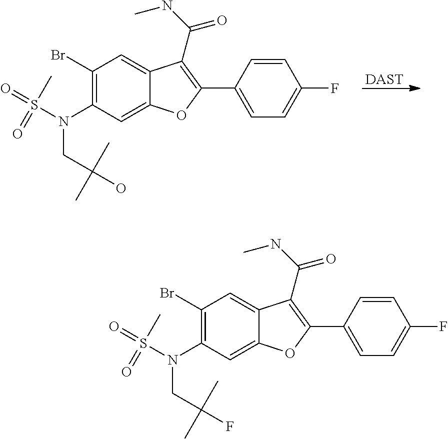

### 5-bromo-2-(4-fluorophenyl)-6-(N-(2-hydroxy-2-methylpropyl)methylsulfonamido)-N-methylbenzofuran-3-carboxamide (450 mg, 0.88 mmol) was dissolved in dichloromethane (6 mL) and the solution was put under nitrogen atmosphere and cooled to −70° C. and stirred for 30 minutes. DAST reagent (283 mg, 1.76 mmol) was added dropwise into the mixture and the reaction was stirred for an additional 3 hours. The reaction was diluted with water and extracted with dichloromethane. The combined organic phases were washed with brine, dried over Na₂SO₄, filtered and concentrated in vacuo to provide 5-bromo-6-(N-(2-fluoro-2-methylpropyl)methylsulfonamido)-2-(4-fluorophenyl)-N-methylbenzofuran-3-carboxamide (320 mg, 71%). ¹H-NMR (CDCl₃, 400 MHz) δ 8.08 (s, 1H), 7.80˜7.84 (m, 2H), 7.73 (s, 1H), 7.13 (t, J=8.0 Hz, 2H), 5.76 (d, J=4.0 Hz, 1H), 3.82˜4.01 (m, 2H), 2.97 (s, 3H), 2.92 (d, J=4.8 Hz, 3H), 1.50 (d, J=22.0 Hz, 3H), 1.29 (d, J=21.2 Hz, 3H). MS (M+H)⁺: 515.

### Step 2—Synthesis of 6-(N-(2-fluoro-2-methylpropyl)methylsulfonamido)-54344-fluorobenzo[d]oxazol-2-yl)-4-methoxyphenyl)-2-(4-fluorophenyl)-N-methylbenzofuran-3-carboxamide

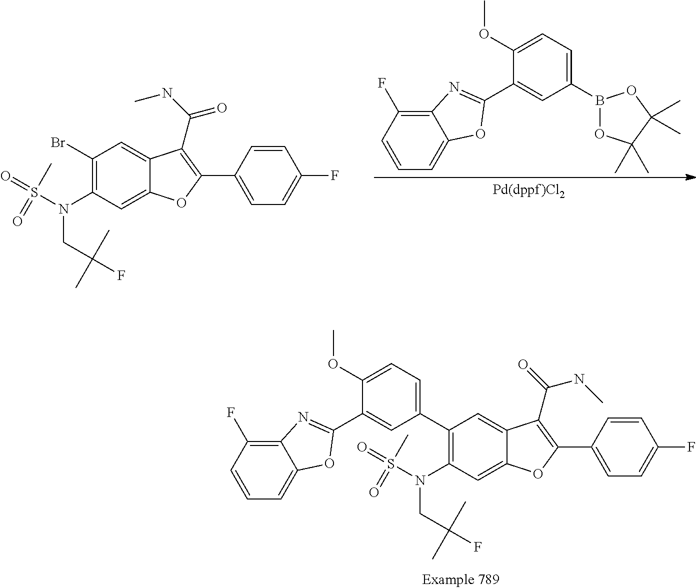

5-bromo-6-(N-(2-fluoro-2-methylpropyl)methylsulfonamido)-2-(4-fluorophenyl)-N-methylbenzofuran-3-carboxamide was converted to the title compound (30 mg, 36%) using the method described in Example 411, Step 12. ¹H-NMR (CDCl₃, 400 MHz) δ 8.02 (s, 1H), 7.85˜7.91 (m, 3H), 7.76 (s, 1H), 7.59 (s, 1H), 7.35 (d, J=8.4 Hz, 1H), 7.23˜7.27 (m, 1H), 7.12˜7.16 (m, 3H), 7.01 (t, J=8.8 Hz, 1H), 5.84 (d, J=4.8 Hz, 1H), 4.03 (s, 3H), 3.67˜3.74 (m, 1H), 3.20 (s, 3H), 2.99˜3.06 (m, 1H), 2.94 (d, J=4.8 Hz, 3H), 1.14 (d, J=21.6 Hz, 3H), 1.01 (d, J=20.8 Hz, 3H). MS (M+H)⁺: 678.

The following compounds of the present invention were made using the method described above and using the appropriate reactants and/or reagents.

| | | | MS |
|---|---|---|---|
| Example | Structure | NMR | (M + H)⁺ |
| 790 | 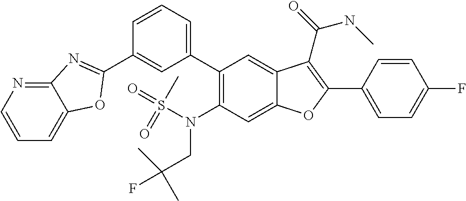 | ¹H-NMR (CDCl₃, 400 MHz) δ 8.56 (d, J = 11.6 Hz, 1H), 8.37 (s, 1H), 7.88~7.93 (m, 2H), 7.84~7.88 (m, 2H), 7.81 (s, 1H), 7.68 (d, J = 9.2 Hz, 1H), 7.65 (s, 1H), 7.35~7.38 (m, 1H), 7.14 (t, J = 8.4 Hz, 2H), 5.84 (d, J = 11.2 Hz, 1H), 3.54 (d, J = 14.8 Hz, 1H), 3.18 (s, 3H), 3.12 (d, J = 15.2 Hz, 1H), 2.94 (s, 3H), 0.99 (s, 6H). | 631 |
| 791 | 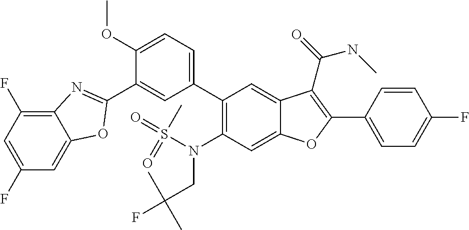 | ¹H-NMR (CDCl₃, 400 MHz) δ 8.19 (d, J = 2.0 Hz, 1H), 7.85~7.91 (m, 3H), 7.76 (s, 1H), 7.58 (s, 1H), 7.11~7.16 (m, 4H), 6.79~6.84 (m, 1H), 5.84 (d, J = 4.4 Hz, 1H), 4.02 (s, 3H), 3.66~3.73 (m, 1H), 3.19 (s, 3H), 3.29~3.06 (m, 1H), 2.93 (d, J = 4.8 Hz, 3H), 1.14 (d, J = 21.2 Hz, 3H), 1.01 (d, J = 20.8 Hz, 3H). | 696 |
| 792 | 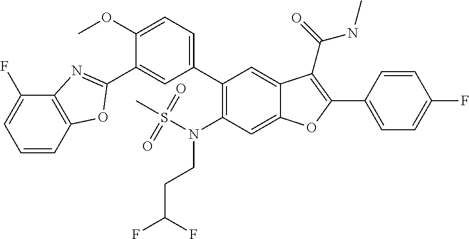 | ¹H-NMR (CDCl₃, 400 MHz) 8.30 (s, 1H), 7.95~7.98 (m, 2H), 7.90 (s, 1H), 7.77 (d, J = 7.2 Hz, 1H), 7.43~7.45 (m, 1H), 7.32~7.36 (m, 1H), 7.19~7.24 (m, 4H), 7.08~7.10 (m, 1H), 6.07 (s, 1H), 5.43~5.72 (m, 1H), 4.11 (s, 3H), 3.53~3.63 (m, 2H), 3.08 (s, 3H), 3.04 (d, J = 4.4 Hz, 3H), 1.91~1.99 (m, 2H). | 682 |
| 793 | 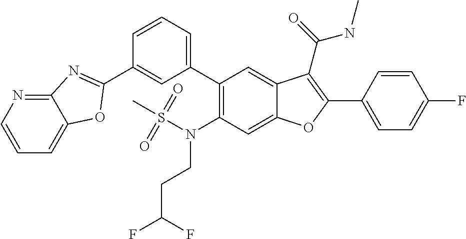 | ¹H~NMR (CDCl₃, 400 MHz) δ 8.50 (s, 1H), 8.32 (s, 1H), 8.27 (d, J = 8.0 Hz, 1H), 7.80~7.89 (m, 4H), 7.69 (d, J = 8.0 Hz, 1H), 7.56~7.58 (m, 2H), 7.23~7.26 (m, 1H), 7.11~7.15 (m, 2H), 6.10 (d, J = 4.4 Hz, 1H), 5.27~5.57 (m, 1H), 3.41~3.51 (m, 2H), 2.93~2.94 (m, 6 H), 1.85~1.86 (m, 2H). | 635 |
| 794 | 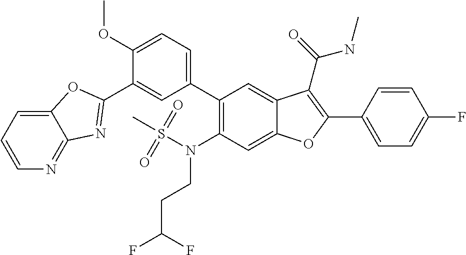 | ¹H-NMR (CDCl₃, 400 MHz) δ 8.52 (d, J = 4.4 Hz, 1H), 8.26 (d, J = 2.0 Hz, 1H), 7.86~7.94 (m, 4H), 7.72~7.74 (m, 1H), 7.59 (s, 1H), 7.27~7.30 (m, 1H), 7.13~7.19 (m, 3H), 6.51 (d, J = 4.4 Hz, 1H), 5.38~5.66 (m, 1H), 4.05 (s, 3H), 3.48~3.57 (m, 2H), 3.02~3.06 (m, 6H), 1.90~1.91 (m, 2H). | 665 |
| 795 | 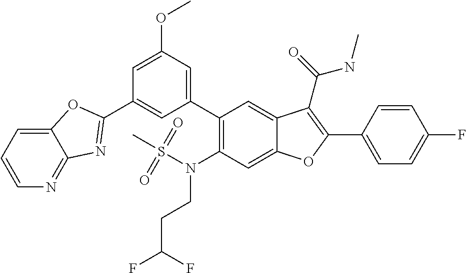 | ¹H-NMR (CDCl₃, 400 MHz) δ 8.52 (s, 1H), 7.83~7.97 (m, 6H), 7.60 (s, 1H), 7.29~7.35 (m, 2H), 7.16~7.20 (m, 2H), 6.48 (d, J = 4.8 Hz, 1H), 5.35~5.63 (m, 1H), 3.94 (s, 3H), 3.46~3.56 (m, 2H), 3.01-3.05 (m, 6H), 1.80-1.95 (m, 2H). | 665 |
| 796 | 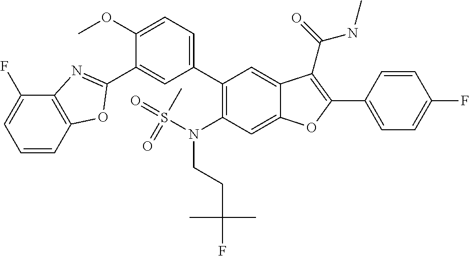 | ¹H-NMR (CDCl₃, 400 MHz) δ 7.13 (s, 1H), 7.81~7.85 (m, 2H), 7.72 (s, 1H), 7.61 (d, J = 8.4 Hz, 1H), 7.48 (s, 1H), 7.17~7.29 (m, 2H), 7.15~7.10 (m, 3H), 6.95 (t, J = 8.8 Hz, 1H), 5.90 (br s, 1H), 3.97 (s, 3H), 3.31~3.52 (m, 2H), 2.90 (d, J = 4.8 Hz, 3H), 2.81 (s, 3H), 1.72 (s, 2H), 1.10~1.15 (m, 2H). | 692 |
| 797 | 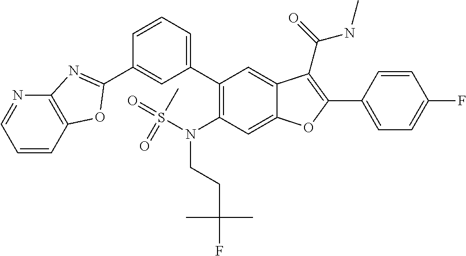 | ¹H-NMR (CDCl₃, 400 MHz) δ 8.51 (d, J = 4.0 Hz, 1H), 8.34 (s, 1H), 8.29 (d, J = 16.8 Hz, 1H), 7.88~7.91 (m, 2H), 7.80~7.83 (m, 2H), 7.70 (d, J = 7.6 Hz, 1H), 7.57~7.60 (m, 1H), 7.23~7.27 (m, 1H), 7.12~7.19 (m, 2H), 5.94 (d, J = 4.4 Hz, 1H), 3.55 (m, 1H), 3.32 (m, 1H), 2.99 (d, J = 5.2 Hz, 3H), 2.88 (s, 1H), 2.85 (s, 3H), 2.81 (s, 1H), 1.14-1.20 (m, 5H). | 645 |
| 798 | 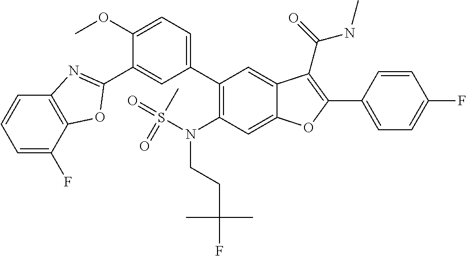 | ¹H-NMR (CDCl₃, 400 MHz) δ 8.17 (d, J = 1.2 Hz, 1H), 7.91~7.88 (m, 2H), 7.79 (s, 1H), 7.69~7.67 (m, 1H), 7.55~7.53 (m, 2H), 7.24~7.17 (m, 1H), 7.15~7.12 (m, 4H), 5.80 (d, J = 2.2 Hz, 1H), 4.01 (s, 3H), 3.62~3.54 (m, 1H), 3.42~3.35 (m, 1H), 2.93 (d, J = 4.4 Hz, 3H), 2.86 (s, 3H), 1.78~1.73 (m, 2H), 1.69~1.66 (m, 3H), 1.18 (d, J = 10.6 Hz, 3H). | 692 |
| 799 | 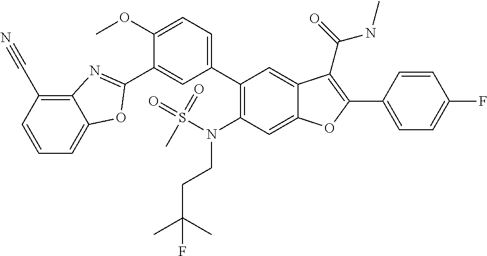 | ¹H-NMR (CDCl₃, 400 MHz) δ 8.23 (d, J = 2.0 Hz, 1H), 7.84~7.88 (m, 2H), 7.75~7.78 (m, 2H), 7.66~7.69 (m, 1H), 7.58 (d, J = 7.6 Hz, 1H), 7.54 (m, 1H), 7.33~7.37 (m, 1H), 7.11 (t, J = 8.8 Hz, 3H), 6.04 (d, J = 4.4 Hz, 1H), 4.02 (s, 3H), 3.59~3.64 (m, 1H), 3.33~3.38 (m, 1H), 2.93 (d, J = 7.2 Hz, 6H), 1.65~1.70 (m, 2H), 1.20 (s, 3H), 1.15 (s, 3H). | 699 |
| 800 | 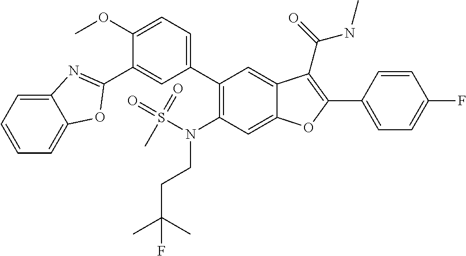 | ¹H-NMR (CDCl₃, 400 MHz) δ 8.21 (s, 1H), 7.92~7.95 (m, 2H), 7.83 (s, 1H), 7.80~7.81 (m, 1H), 7.71~7.79 (m, 1H), 7.58 (s, 1H), 7.54~7.57 (m, 1H), 7.35 (d, J = 2.8 Hz, 2H), 7.16~7.19 (m, 3H), 5.95 (d, J = 4.4 Hz, 1H), 4.07 (s, 3H), 3.60~3.65 (m, 1H), 3.38~3.43 (m, 1H), 2.98 (d, J = 4.8 Hz, 3H), 2.91 (s, 3H), 1.71~1.83 (m, 2H), 1.25 (s, 3H), 1.20 (s, 3H). | 674 |
| 801 | 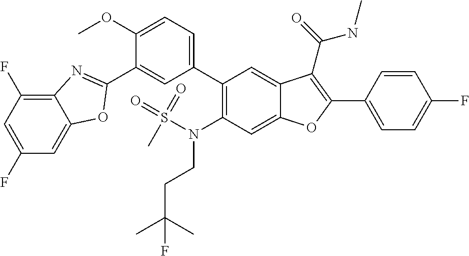 | ¹H-NMR (CDCl₃, 400 MHz) δ 8.16 (s, 1H), 7.86~7.89 (m, 1H), 7.79 (s, 1H), 7.68 (d, J = 7.6 Hz, 1H), 7.55 (s, 1H), 7.12~7.16 (m, 4H), 6.82~6.87 (m, 1H), 6.24 (d, J = 2.4 Hz, 1H), 4.05 (s, 3H), 3.58~3.63 (m, 1H), 3.36~3.41 (m, 1H), 2.99 (d, J = 4.0 Hz, 3H), 2.92 (s, 3H), 1.69~1.81 (m, 2H), 1.24 (s, 3H), 1.18 (s, 3H). | 710 |
| 802 | 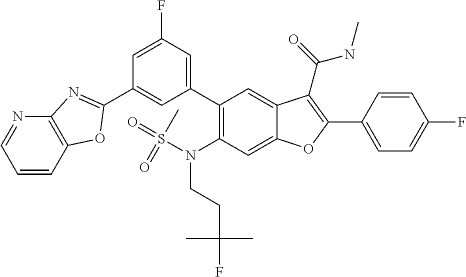 | ¹H-NMR (CDCl₃, 400 MHz) δ 8.63 (d, J = 2.4 Hz, 1H), 8.24 (s, 1H), 8.07 (s, 4H), 7.97 (d, J = 15.4 Hz, 1H), 7.96~7.91 (m, 2H), 7.53 (d, J = 4.4 Hz, 1H), 7.38~7.21 (m, 2H), 5.90 (d, J = 1.8 Hz, 1H), 3.64 (s, 2H), 3.39 (d, J = 1.8 Hz, 2H), 3.00 (d, J = 2.4 Hz, 3H), 2.98 (s, 3H), 1.28 (s, 3H), 1.23 (s, 3H). | 663 |
| 803 | 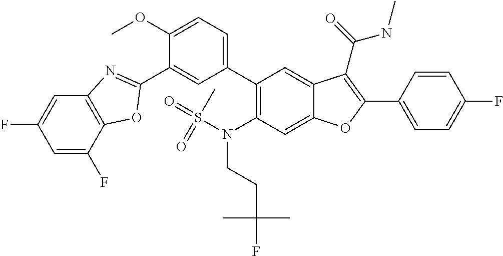 | ¹H-NMR (CDCl₃, 400 MHz) δ 8.15 (s, 1H), 7.88 (t, J = 2.8 Hz, 2H), 7.77 (d, J = 9.6 Hz, 1H), 7.71~7.67 (m, 1H), 7.54~7.53 (m, 1H), 7.25~7.24 (m, 1H), 7.14 (d, J = 4.2 Hz, 4H), 5.77 (d, J = 7.2 Hz, 1H), 4.03 (s, 3H), 3.70~3.51 (m, 2H), 3.50-3.26 (m, 2H), 2.93 (d, J = 2.2 Hz, 3H), 2.87 (s, 3H), 1.31 (s, 3H), 1.21 (s, 3H), 1.16 (s, 3H). | 710 |
| 804 | 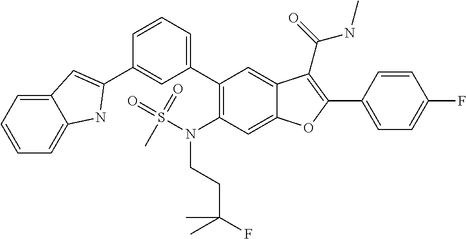 | ¹H-NMR (CDCl₃, 400 MHz) δ 9.20 (s, 1H), 7.89 (s, 1H), 7.82~7.83 (m, 2H), 7.75 (s, 1H), 7.65 (d, J = 8.0 Hz, 1H), 7.53 (s, 1H), 7.33~7.39 (m, 3H), 7.23 (d, J = 7.6 Hz, 1H), 7.02~7.11 (m, 4H), 6.75 (s, 1H), 5.94 (d, J = 4.8 Hz, 1H), 3.42~3.50 (m, 1H), 3.05~3.10 (m, 1H), 3.04 (s, 3H), 2.85 (s, 3H), 1.42~1.57 (m, 2H), 1.03~1.11 (m, 6H). | 642 |
| 805 | 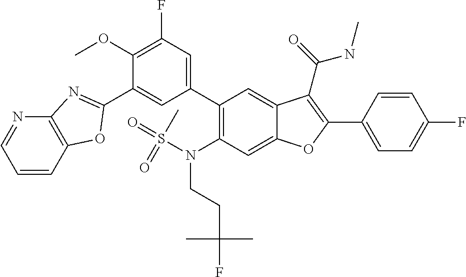 | ¹H~NMR (CDCl₃, 400 MHz) δ 8.54 (s, 1H), 8.01 (s, 1H), 7.79 (t, J = 14.8 Hz, 4H), 7.54~7.45 (m, 2H), 7.30 (s, 1H), 7.19~7.12 (m, 2H), 5.97 (s, 1H), 4.13 (s, 3H), 3.96~3.39 (m, 2H), 3.23 (d, J = 2.2 Hz, 2H), 2.95 (d, J = 3.0 Hz, 3H), 1.68 (d, J = 1.6 Hz, 3H), 1.21 (s, 3H), 1.16 (s, 3H). | 693 |
| 806 | 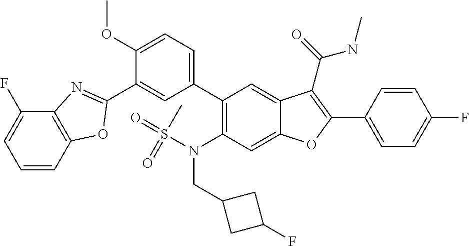 | ¹H-NMR (MeOD, 400 MHz) δ 8.26 (s, 1H), 7.96~8.00 (m, 2H), 7.85~7.89 (m, 2H), 7.79 (s, 1H), 7.68~7.71 (m, 1H), 7.49~7.51 (m, 1H), 7.33~7.42 (m, 2H), 7.24~7.28 (m, 2H), 7.13~7.18 (m, 1H), 4.59 (s, 3H), 4.06 (s, 3H), 3.22 (s, 2H), 2.93 (s, 3H), 1.27 (s, 3H), 0.86~0.88 (m, 1H), 0.32~0.5 (m, 3H). | 690 |
| 807 | 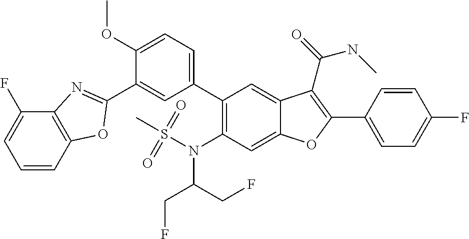 | ¹H-NMR (CDCl₃, 400 MHz) δ 8.21 (s, 1H), 7.54~7.91 (m, 5H), 7.22~7.37 (m, 5H), 7.00 (t, J = 8.8 Hz, 1H), 5.83 (br s, 1H), 4.85~5.00 (m, 1H), 4.20~4.52 (m, 2H), 3.88~4.03 (m, 4H), 3.18 (s, 2H), 2.90 (d, J = 10.6 Hz, 4H). | 682 |
| 808 | 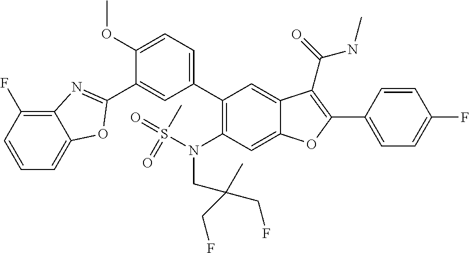 | ¹H-NMR (MeOD, 400 MHz) δ 8.32 (s, 1H), 7.87~7.90 (m, 4H), 7.63 (s, 1H), 7.41 (d, J = 8.4 Hz, 1H), 7.26~7.31 (m, 2H), 7.15~7.19 (m, 3H), 7.04~7.08 (m, 1H), 3.92~3.98 (m, 5H), 3.80~3.84 (m, 2H), 3.51~3.55 (m, 1H), 3.32~3.36 (m, 1H), 3.19 (s, 3H), 2.85 (s, 3H), 1.18 (s, 3H). | 710 |
| 809 | 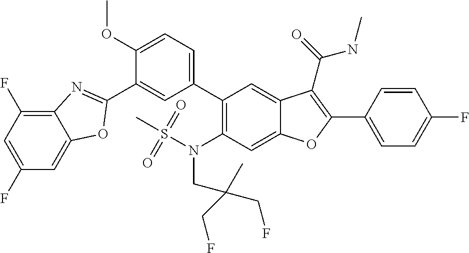 | ¹H-NMR (MeOD, 400 MHz) δ 8.31 (s, 1H), 7.87~7.91 (m, 4H), 7.62 (s, 1H), 7.16~7.30 (m, 4H), 6.96~7.01 (m, 1H), 3.96~4.03 (m, 5H), 3.78~3.84 (m, 2H), 3.51~3.55 (m, 1H), 3.32~3.36 (m, 1H), 3.19 (s, 3H), 2.85 (s, 3H), 1.19 (s, 3H). | 728 |
| 810 | 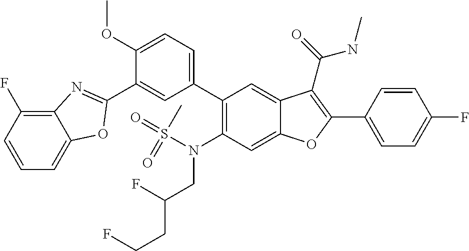 | ¹H-NMR (CDCl₃, 400 MHz) δ 8.23 (s, 1H), 7.83~7.94 (m, 4H), 7.57 (s, 1H), 7.40 (d, J = 7.6 Hz, 1H), 7.04~7.29 (m, 5H), 6.04 (s, 1H), 4.34~4.61 (m, 3H), 4.07 (s, 3H), 3.85~3.88 (m, 1H), 3.23 (s, 2H), 2.83~2.99 (m, 5H), 1.68~1.85 (m, 2H). | 696 |

### Example 811

### 6-(N-(4-fluoro-2-hydroxybutyl)methylsulfonamido)-5-(3-(4-fluorobenzoldloxazol-2-yl)-4-methoxyphenyl)-2-(4-fluorophenyl)-N-methylbenzofuran-3-carboxamide

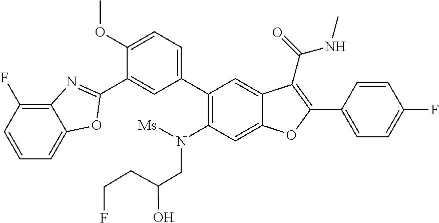

### Step 1—Synthesis of 4-0′-(5-(3-(4-fluorobenzo[d]oxazol-2-yl)-4-methoxyphenyl)-2-(4-fluorophenyl)-3-(methylcarbamoyl)benzofuran-6-yl)methylsulfonamido)-3-hydroxybutyl 4-methylbenzenesulfonate

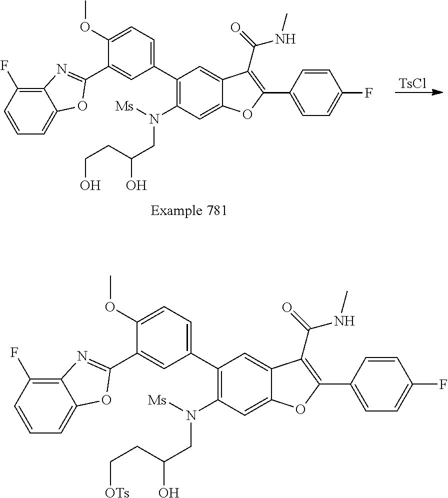

To a 0° C. solution of the compound of Example 581 (100 mg, 0.16 mmol), DMAP (10 mg) and TEtOAc (0.1 mL) in dichloromethane (1 mL) was added TsCl (30.8 mg, 0.16 mmol) and the reaction was allowed to stir at room temperature for 5 hours. Water was added, and the reaction mixture was extracted with dichloromethane. The combined extracts were washed with brine, dried over Na₂SO₄, filtered and concentrated in vacuo and the residue obtained was purified using prep-TLC (petroleum ether:EtOAc=1:1) to provide 4-(N-(5-(3-(4-fluorobenzo[d]oxazol-2-yl)-4-methoxyphenyl)-2-(4-fluorophenyl)-3-(methylcarbamoyl)benzofuran-6-yl)methylsulfonamido)-3-hydroxybutyl 4-methylbenzenesulfonate (70 mg, 58%). ¹H-NMR (CDCl₃, 400 MHz) 8.8.09˜8.31 (m, 1H), 7.92 (s, 2H), 7.99 (d, J=2.0 Hz, 2H), 7.61˜7.72 (m, 3H), 7.03˜7.45 (m, 8H), 6.06˜6.22 (m, 1H), 4.14˜4.30 (m, 2H), 4.06 (s, 3H), 3.56˜3.93 (m, 3H), 2.70˜3.06 (m, 6H), 2.33˜2.40 (m, 3H), 1.60˜1.78 (m, 2H). MS (M+H)⁺: 846.

### Step 2—Synthesis of 6-(N-(4-fluoro-2-hydroxybutyl)methylsulfonamido)-5-(3-(4-fluorobenzo[d]oxazol-2-yl)-4-methoxyphenyl)-2-(4-fluorophenyl)-N-methylbenzofuran-3-carboxamide

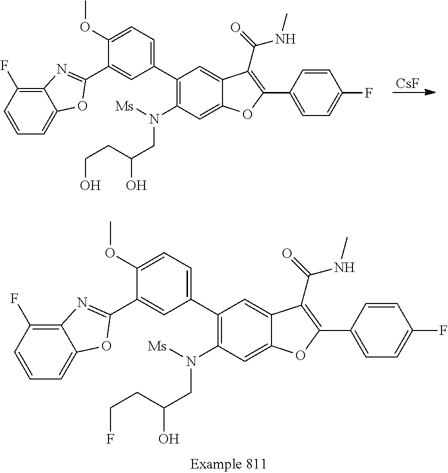

A mixture of 4-(N-(5-(3-(4-fluorobenzo[d]oxazol-2-yl)-4-methoxyphenyl)-2-(4-fluorophenyl)-3-(methylcarbamoyl)benzofuran-6-yl)methylsulfonamido)-3-hydroxybutyl 4-methylbenzenesulfonate (50 mg, 0.06 mmol), CsF (27 mg, 0.12 mmol) in t-BuOH (2 mL) was heated to 80° C. and allowed to stir at this temperature for 5 hours. Then water was added, and the reaction mixture was extracted with dichloromethane. The combined extracts were washed with brine, dried over Na₂SO₄, filtered and concentrated in vacuo, and the residue obtained was purified using prep-HPLC to provide the title compound (30 mg, 73%). ¹H-NMR (CDCl₃, 400 MHz) δ 8.16˜8.37 (m, 1H), 7.81˜7.93 (m, 4H), 7.72 (d, J=7.2 Hz, 1H), 7.66 (s, 1H), 7.29˜7.46 (m, 2H), 7.04˜7.20 (m, 4H), 5.89˜6.61 (m, 1H), 4.46˜4.85 (m, 2H), 4.05 (s, 3H), 3.57˜3.91 (m, 2H), 2.97˜3.31 (m, 5H), 2.67 (s, 2H), 1.62˜1.69 (m, 2H). MS (M+H)⁺: 694.

The following compounds of the present invention were made using the method described above and using the appropriate reactants and/or reagents.

| | | | MS |
|---|---|---|---|
| Example | Structure | NMR | (M + H)⁺ |
| 812 | 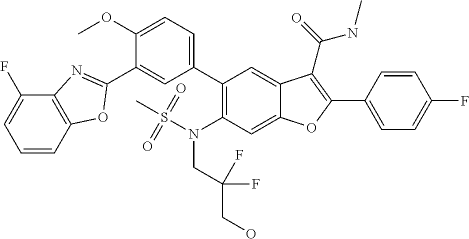 | ¹H-NMR (CDCl₃, 400 MHz) δ 8.21 (s, 1H), 7.79~7.94 (m, 4H), 7.61 (s, 1H), 7.41 (d, J = 8.4 Hz, 1H), 7.04~7.32 (m, 5H), 6.20 (d, J = 4.4 Hz, 1H), 3.97~4.04 (m, 4H), 3.45~3.68 (m, 3H), 3.17 (s, 3H), 2.99 (d, J = 4.4 Hz, 3H). | 698 |
| 813 | 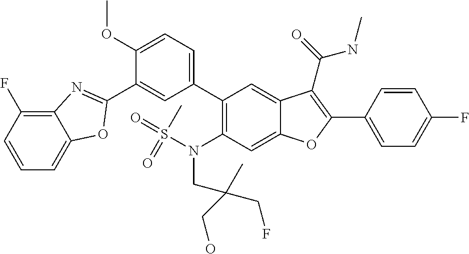 | ¹H-NMR (MeOD, 400 MHz) δ 8.40 (s, 1H), 7.94~7.98 (m, 4H), 7.69 (s, 1H), 7.50 (d, J = 8.0 Hz, 1H), 7.34~7.42 (m, 2H), 7.23~7.27 (m, 2H), 7.12~7.17 (m, 1H), 4.59 (s, 3H), 4.02~4.06 (m, 3H), 3.81~3.93 (m, 2H), 3.53~3.60 (m, 1H), 3.33~3.41 (m, 1H), 3.10~3.11 (m, 2H), 2.93 (s, 3H), 1.26 (s, 3H). | 708 |
| 814 | 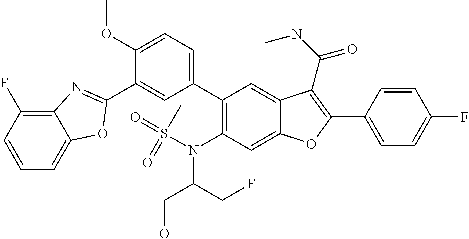 | ¹H-NMR (CDCl₃, 400 MHz) δ 7.86 (d, J = 7.6 Hz, 1H), 7.81~7.60 (m, 5H), 7.45~7.24 (m, 2H), 7.14 (t, J = 8.8 Hz, 3H), 7.08~7.01 (m, 1H), 5.84 (t, J = 7.2 Hz, 1H), 4.43~4.11 (m, 2.5H), 4.01 (d, J = 4.4 Hz, 3H), 3.89~3.50 (m, 2.5H), 3.01 (s, 1H), 2.93. (s, 3H), 2.64 (d, J = 14.8 Hz, 2H). | 680 |
| 815 | 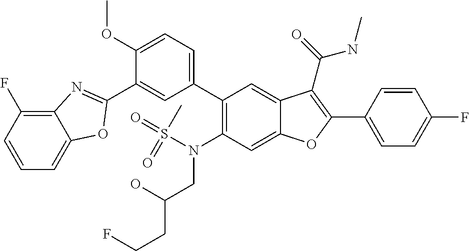 | ¹H-NMR (CDCl₃, 400 MHz) δ 8.16~8.37 (m, 1H), 7.81~7.93 (m, 4H), 7.72 (d, J = 7.2 Hz, 1H), 7.66 (s, 1H), 7.29~7.46 (m, 2H), 7.04~7.20 (m, 4H), 5.89~6.61(m, 1H), 4.46~4.85 (m, 2H), 4.05 (s, 3H), 3.57~3.91 (m, 2H), 2.97~3.31 (m, 5H), 2.67 (s, 2H), 1.62~1.69 (m, 2H). | 694 |

### Example 816

### 6-(N-(4-cyano-2-hydroxybutyl)methylsulfonamido)-5-(344-fluorobenzoldloxazol-2-yl)-4-methoxyphenyl)-2-(4-fluorophenyl)-N-methylbenzofuran-3-carboxamide

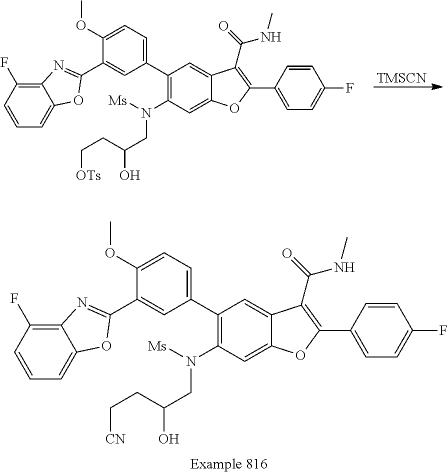

A mixture of 4-(N-(5-(3-(4-fluorobenzo[d]oxazol-2-yl)-4-methoxyphenyl)-2-(4-fluorophenyl)-3-(methylcarbamoyl)benzofuran-6-yl)methylsulfonamido)-3-hydroxybutyl 4-methylbenzenesulfonate (50 mg, 0.06 mmol, made as described in Example 444), TMSCN (11.7 mg, 0.12 mmol) and TBAF (32.3 mg, 0.12 mmol) in CH₃CN (2 mL) was heated to 70° C. and allowed to stir at this temperature for 5 hours. Then water was added and the resulting solution was extracted with dichloromethane. The combined extracts were washed with brine, dried over Na₂SO₄, filtered and concentrated in vacuo, and the resulting residue was purified using prep-HPLC to provide the title compound (35 mg, 85%). ¹H-NMR (CDCl₃, 400 MHz) δ 8.51 (s, 1H), 7.61˜7.95 (m, 5H), 7.08˜7.48 (m, 6H), 5.91 (d, J=4.8 Hz, 1H), 3.48˜4.11 (m, 6H), 2.98 (d, J=4.8 Hz, 3H), 2.43˜2.88 (m, 5H), 1.71˜1.73 (m, 2H). MS (M+H)⁺: 701.

### Example 817

### 6-(N-(3-cyano-2-hydroxypropyl)methylsulfonamido)-5-(3-(4-fluorobenzo[d]oxazol-2-yl)-4-methoxyphenyl)-2-(4-fluorophenyl)-N-methylbenzofuran-3-carboxamide

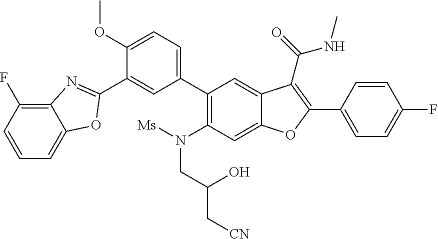

### Step 1—Synthesis of 5-bromo-6-(N-(3-cyano-2-hydroxypropyl)methylsulfonamido)-2-(4-fluorophenyl)-N-methylbenzofuran-3-carboxamide

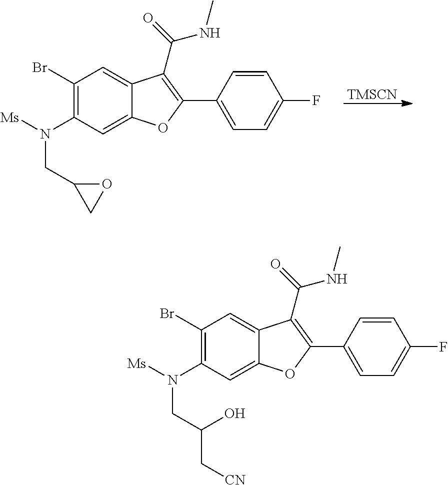

A mixture of 5-bromo-2-(4-fluorophenyl)-N-methyl-6-(N-(oxiran-2-ylmethyl)methylsulfonamido)benzofuran-3-carboxamide (1.05 g, 2.1 mmol, prepared from Compound 411G as described in Example 411, Step 6), TMSCN (837 mg, 8.5 mmol) and TBAF (2.20 g, 8.5 mmol) in THF (50 mL) was heated to reflux (80° C.) and allowed to stir at this temperature for 2 hours. The reaction was cooled to room temperature, diluted with water and extracted with EtOAc. The organic extract was washed with water and brine, dried over Na₂SO₄, filtered and concentrated in vacuo to provide 5-bromo-6-(N-(3-cyano-2-hydroxypropyl)methylsulfonamido)-2-(4-fluorophenyl)-N-methylbenzofuran-3-carboxamide (1.35 g), which was used without further purification.

### Step 2—Synthesis of 6-(N-(3-cyano-2-hydroxypropyl)methylsulfonamido)-5-(3-(4-fluorobenzo[d]oxazol-2-yl)-4-methoxyphenyl)-2-(4-fluorophenyl)-N-methylbenzofuran-3-carboxamide

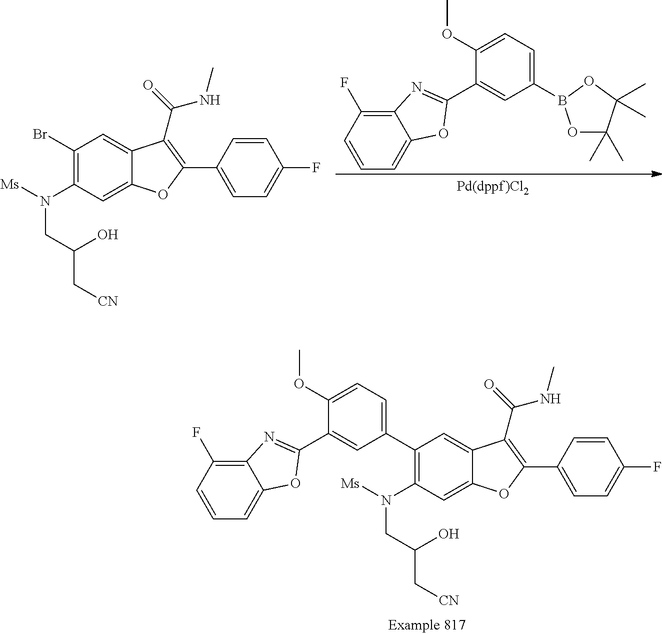

5-bromo-6-(N-(3-cyano-2-hydroxypropyl)methylsulfonamido)-2-(4-fluorophenyl)-N-methylbenzofuran-3-carboxamide was converted to the title compound (18 mg, 10.6%) using the method described in Example 411, Step 12. ¹H-NMR (CDCl₃, 400 MHz) δ 8.08˜8.35 (m, 1H), 7.59˜7.91 (m, 5H), 7.03˜7.47 (m, 6H), 6.00˜6.14 (m, 1H), 3.83˜4.33 (m, 5H), 3.47˜3.72 (m, 1H), 2.41˜3.03 (m, 8H). MS (M+H)⁺: 687.

The following compounds of the present invention were made using the method described above and using the appropriate reactants and/or reagents.

| | | | MS |
|---|---|---|---|
| Example | Structure | NMR | (M + H)⁺ |
| 818 | 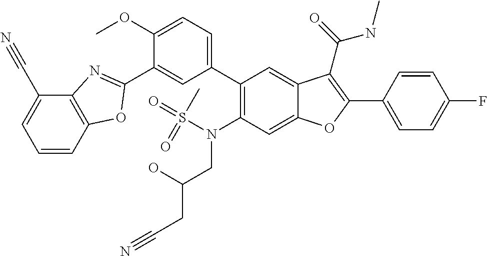 | ¹H-NMR (CDCl₃, 400 MHz) δ 8.13~8.56 (m, 1H), 7.84~7.93 (m, 4H), 7.61~7.78 (m, 3H), 7.41~7.49 (m, 1H), 7.17~7.26 (m, 3H), 5.87~5.99 (m, 1H), 4.27~4.43 (m, 1H), 4.07 (s, 3H), 3.61~4.03 (m, 2H), 2.55~3.00 (m, 8H). | 694 |
| 819 | 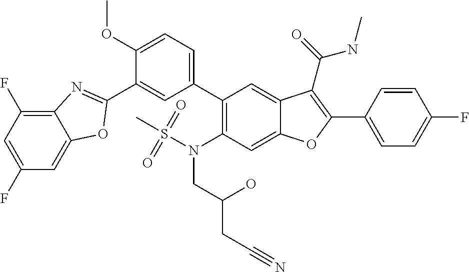 | ¹H-NMR (CDCl₃, 400 MHz) δ 8.05~8.23 (m, 1H), 7.58~7.89 (m, 5H), 7.10~7.22 (m, 4H), 6.84~6.93 (m, 1H), 6.03~6.17 (m, 1H), 3.65~4.24 (m, 5H), 3.42~3.43 (m, 1H), 2.25~3.08 (m, 8H). | 705 |
| 820 | 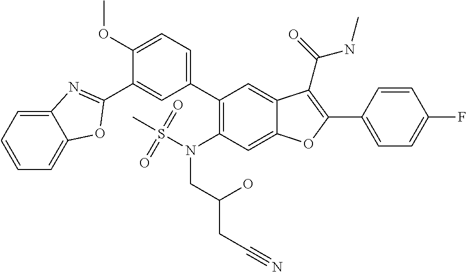 | ¹H-NMR (CDCl₃, 400 MHz) δ 8.02~8.04 (m, 1H), 7.89~7.97 (m, 4H), 7.56~7.79 (m, 3H), 7.37~7.39 (m, 2H), 7.16~7.20 (m, 3H), 5.97~6.03 (m, 1H), 4.14~4.40 (m, 1H), 3.55~3.98 (m, 5H), 2.87~2.98 (m, 4H), 2.41~2.54 (m, 4H). | 669 |
| 821 | 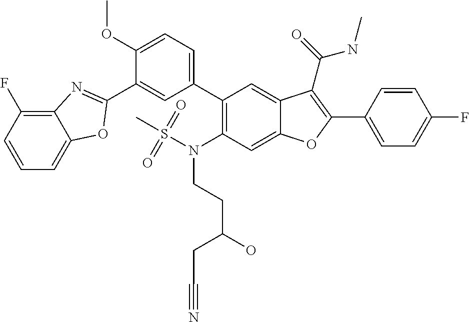 | ¹H-NMR (CDCl₃, 400 MHz) δ 8.01~7.65 (m, 1H), 7.80~7.81 (m, 3H), 7.67~7.73 (m, 1H), 7.47 (d, J = 6.8 Hz, 1H), 7.35 (d, J = 8.4 Hz, 1H), 7.19~7.26 (m, 1H), 7.03~7.08 (m, 3H), 6.97~7.01 (m, 1H), 6.24~6.29 (m, 1H), 4.05 (s, 3H), 3.25~3.76 (m, 4H), 3.00 (s, 2H), 2.91 (s, 3H), 2.77 (s, 1H), 2.37 (s, 1H), 2.06 (s, 1H), 1.34~1.38 (m, 1H). | 701 |
| 822 | 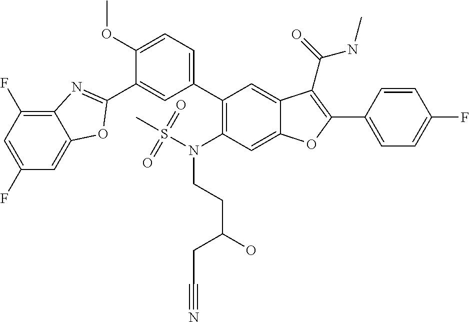 | ¹H-NMR (CDCl₃, 400 MHz) δ 8.10~8.13 (m, 1H), 7.84~7.92 (m, 3H), 7.79~7.84 (m, 1H), 7.57 (s, 1H), 7.17~7.21 (m, 4H), 6.86~6.91 (m, 1H), 6.05 (s, 1H), 4.07 (s, 3H), 3.33~3.99 (m, 4H), 3.09 (s, 2H), 2.98 (s, 3H), 2.87 (s, 1H), 2.47 (s, 1H), 2.15 (s, 1H), 1.46 (s, 1H). | 719 |

### Example 823

### 5-(3-(4-fluorobenzo[d]oxazol-2-yl)-4-methoxyphenyl)-2-(4-fluorophenyl)-6-(N42-hydroxy-3-(1H-imidazol-1-yl)propyl)methylsulfonamido)-N-methylbenzofuran-3-carboxamide

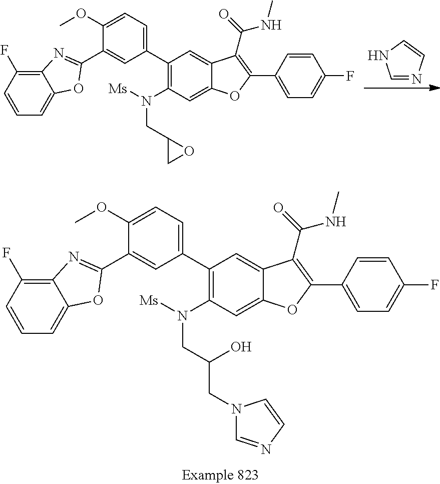

To a microwave tube was added 5-bromo-2-(4-fluorophenyl)-N-methyl-6-(N-(oxiran-2-yl-methyl)methylsulfonamido)benzofuran-3-carboxamide (43 mg, 0.07 mmol, prepared according to the method described in Example 440), imidazole (11 mg, 0.16 mmol), Cs₂CO₃ (53 mg, 0.16 mmol) and 5 mL of DMF. The reaction was placed in a commercial microwave for 30 minutes during which time the reaction temperature reached 120° C. The reaction mixture was then cooled to RT and water was added and the solution was extracted with EtOAc. The organic extract was washed with H₂O and brine, dried over Na₂SO₄, filtered and concentrated in vacuo. The resulting residue was purified using prep-HPLC to provide the title compound (38 mg, 80.3%). ¹H-NMR (CDCl₃, 400 MHz) δ 8.19˜8.06 (m, 1H), 7.77˜7.60 (m, 5H), 7.49 (s, 1H), 7.38˜7.35 (m, 4H), 7.07˜6.98 (m, 1.5H), 6.92˜6.67 (m, 1.5H), 5.29 (s, 1H), 4.13 (s, 1H), 4.00 (t, J=9.2 Hz, 3H), 3.88˜3.55 (m, 4H), 3.11 (d, J=2.4 Hz, 2H), 3.08˜2.92 (m, 3H), 2.83 (d, J=2.0 Hz, 2H). MS (M+H)⁺: 728.

The following compounds of the present invention were made using the method described above and using the appropriate reactants and/or reagents.

| | | | MS |
|---|---|---|---|
| Example | Structure | NMR | (M + H)⁺ |
| 824 | 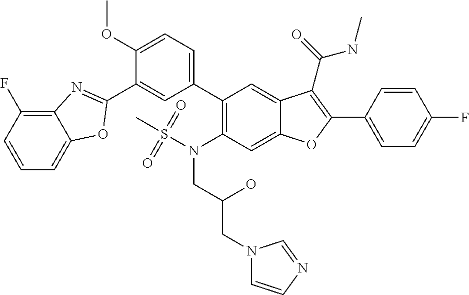 | ¹H-NMR (CDCl₃, 400 MHz) δ 8.19~8.06 (m, 1H), 7.77~7.60 (m, 5H), 7.49 (s, 1H), 7.38~7.35 (m, 4H), 7.07~6.98 (m, 1.5H), 6.92~6.67 (m, 1.5H), 5.29 (s, 1H), 4.13 (s, 1H), 4.00 (t, J = 9.2 Hz, 3H), 3.88~3.55 (m, 4H), 3.11 (d, J = 2.4 Hz, 2H), 3.08~2.92 (m, 3H), 2.83 (d, J = 2.0 Hz, 2H). | 728 |
| 825 | 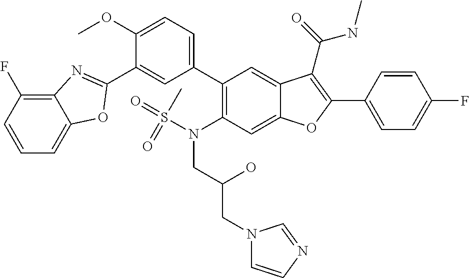 | ¹H-NMR (CDCl₃, 400 MHz) δ 8.31~7.99 (m, 2H), 7.90~7.81 (m, 2H), 7.81~7.79 (m, 2H), 7.74~7.69 (m, 1H), 7.62~7.59 (m, 1H), 7.43~7.42 (m, 1H), 7.41~7.31 (m, 1H), 7.29~7.03 (m, 4H), 6.35~6.26 (m, 1H), 4.31~4.08 (m, 2H), 4.06~3.55 (m, 6H), 3.27~3.11 (m, 2H), 2.97 (d, J = 0.4 Hz, 3H), 2.71 (s, 1H). | 729 |
| 826 | 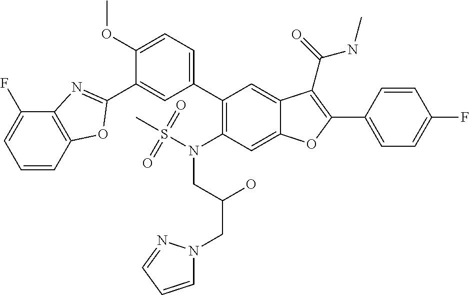 | ¹H-NMR (CDCl₃, 400 MHz) δ 8.16 (d, J = 4.4 Hz, 1H), 7.81 (d, J = 1.2 Hz, 3H), 7.83~7.74 (m, 1.5H), 7.74~7.63 (m, 1H), 7.44~7.33 (m, 2.5H), 7.32~7.29 (m, 1H), 7.24 (d, J = 4.4 Hz, 3H), 7.15 (t, J = 8.8 Hz, 1H), 6.16 (d, J = 1.2 Hz, 2H), 4.25~4.12 (m, 1H), 4.10-4.05 (m, 3H), 3.95~3.85 (m, 2H), 3.59~3.47 (m, 1H), 3.47~3.24 (m, 2H), 3.00~2.95 (m, 3H), 2.89~2.81 (m, 2H). | 728 |
| 827 | 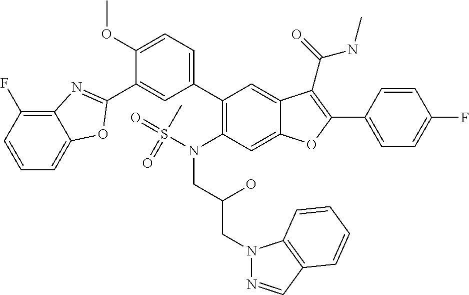 | ¹H-NMR (CDCl₃, 400 MHz) δ 8.17 (d, J = 5.2 Hz, 1H), 7.96~7.72 (m, 5H), 7.66~7.58 (m, 2H), 7.45~7.40 (m, 3H), 7.35~7.05 (m, 6H), 5.95 (d, J = 0.4 Hz, 1H), 4.39~4.37 (m, 1H), 4.20~4.14 (m, 2H), 4.12~4.05 (m, 3H), 3.71~3.66 (m, 1.5H), 3.18 (t, J = 1.4 Hz, 2.5H), 2.99 (t, J = 0.4 Hz, 3H), 2.77 (s, 1H). | 778 |
| 828 | 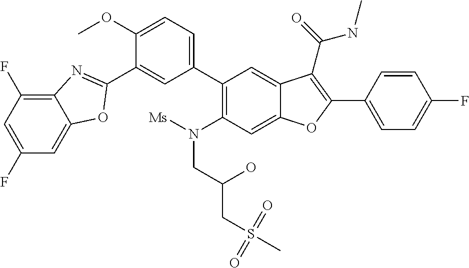 | ¹H-NMR (CDCl₃, 400 MHz) δ 8.16~8.33 (m, 1H), 7.79~7.93 (m, 4H), 7.65 (s, 1H), 7.17~7.23 (m, 4H), 6.86-6.92 (m, 1H), 5.97 (s, 1H), 4.31 (s, 1H), 4.06 (s, 3H), 3.65~3.83 (m, 2H), 2.86~3.24 (m, 11H). | 758 |

### Example 829

### 2-(4-fluorophenyl)-N-methyl-5-(3-(oxazolo[4,5-b]pyridin-2-yl)phenyl)-6-(N-(2-(phenylamino)propyl)methylsulfonamido)benzofuran-3-carboxamide

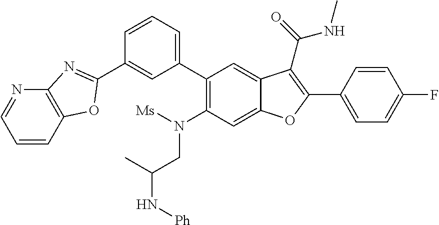

### Step 1—Synthesis of 1-(N-(2-(4-fluorophenyl)-3-(methylcarbamoyl)-5-(3-(oxazol o[4,5-b]pyridin-2-yl)phenyl)benzofuran-6-yl)methylsulfonamido)propan-2-yl methanesulfonate

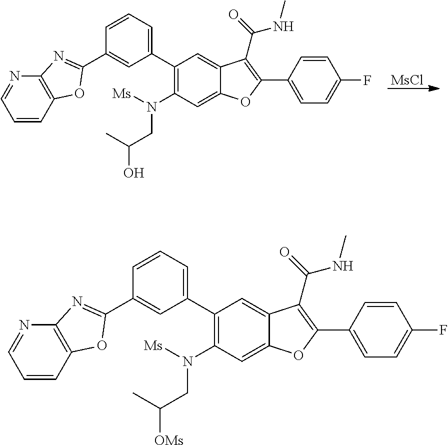

To a solution of 2-(4-fluorophenyl)-6-(N-(2-hydroxypropyl)methylsulfonamido)-N-methyl-5-(3-(oxazolo[4,5-b]pyridin-2-yl)phenyl)benzofuran-3-carboxamide (147 mg, 0.24 mmol, prepared according to the method described in Example 440) in dichloromethane (1.5 mL) was added Et₃N (30 mg, 0.29 mmol). The reaction was cooled to 0° C. and MsCl (30 mg, 0.26 mmol) was added dropwise. The reaction was warmed to 25° C. and allowed to stir at this temperature for 1 hour, then the reaction mixture was extracted with dichloromethane, and the organic extract was concentrated in vacuo. The residue obtained was purified using prep-TLC (petroleum ether:EtOAc=2:1) to provide 1-(N-(2-(4-fluorophenyl)-3-(methylcarbamoyl)-5-(3-(oxazolo[4,5-b]pyridin-2-yl)phenyl)benzofuran-6-yl)methylsulfonamido)propan-2-yl methanesulfonate (0.1 g, 85%) as yellow solid. ¹H-NMR (MeOD, 400 MHz) δ 8.52˜8.56 (m, 2H), 8.39˜8.40 (m, 1H), 8.15˜8.17 (m, 1H), 7.96˜8.03 (m, 4H), 7.71˜7.75 (m, 2H), 7.46˜7.50 (m, 1H), 7.27˜7.31 (m, 2H), 4.62 (s, 2H), 4.07˜4.12 (m, 1H), 3.03 (s, 3H), 2.94 (s, 3H), 2.01 (s, 3H), 2.01 (s, 3H), 1.28 (s, 3H). MS (M+H)⁺: 693.

### Step 2—Synthesis of 2-(4-fluorophenyl)-N-methyl-5-(3-(oxazolo[4,5-b]pyridin-2-yl)phenyl)-6-(N-(2-(phenylamino)propyl)methylsulfonamido)benzofuran-3-carboxamide

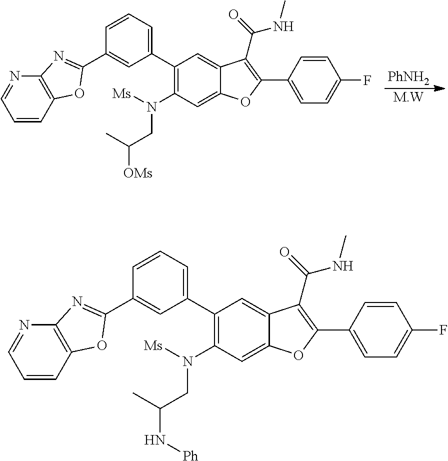

To a solution of 1-(N-(2-(4-fluorophenyl)-3-(methylcarbamoyl)-5-(3-(oxazolo[4,5-b]pyridin-2-yl)phenyl)benzofuran-6-yl)methylsulfonamido)propan-2-yl methanesulfonate (100 mg, 0.14 mmol) in MeCN (2 mL) was added Et₃N (1 mL), PhNH₂ (130 mg, 0.14 mmol) and DMAP (12 mg) and the mixture was placed in a commercial microwave oven and irradiated for 1 hour, during which time the reaction temperature went to 120° C. The reaction was cooled to RT, diluted with dichloromethane, and the resulting solution was washed with brine, dried over Na₂SO₄, filtered and concentrated in vacuo. The residue obtained was purified using prep-HPLC to provide the title compound (30 mg, 30%) as white solid. ¹H-NMR (MeOD, 400 MHz) δ 8.51˜8.55 (m, 2H), 8.35˜8.39 (m, 1H), 8.14˜8.16 (m, 1H), 7.93˜8.00 (m, 4H), 7.68˜7.77 (m, 2H), 7.30˜7.49 (m, 1H), 7.25˜7.30 (m, 2H), 7.25˜7.30 (m, 1H), 6.26˜6.97 (m, 4H), 3.46˜3.70 (m, 2H), 3.17˜3.19 (m, 4H), 2.92 (s, 3H), 1.03˜1.05 (m, 1H), 0.74˜0.76 (m, 2H). MS (M+H)⁺: 689.

The following compounds of the present invention were made using the method described above and using the appropriate reactants and/or reagents.

| | | | MS |
|---|---|---|---|
| Example | Structure | NMR | (M + H)⁺ |
| 830 | 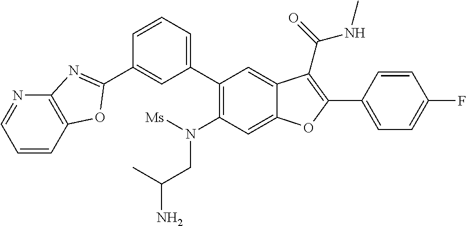 | ¹H-NMR (MeOD, 400 MHz) δ 8.54~8.56 (m, 2H), 8.38~8.42 (m, 1H), 8.16~8.18 (m, 1H), 7.97~8.00 (m, 4H), 7.76 (s, 1H), 7.47~7.51 (m, 1H), 7.27~7.31 (m, 2H), 3.46 (s, 1H), 3.23 (s, 1H), 3.11~3.15 (m, 3H), 2.92 (s, 3H), 1.15~1.17 (m, 1H), 0.81~0.83 (m, 3H). | 614 |

### Example 831

### 2-(4-fluorophenyl)-N-methyl-5-(3-(oxazolo[4,5-b]pyridin-2-yl)phenyl)-6-(N-(piperidin-4-ylmethyl)methylsulfonamido)benzofuran-3-carboxamide

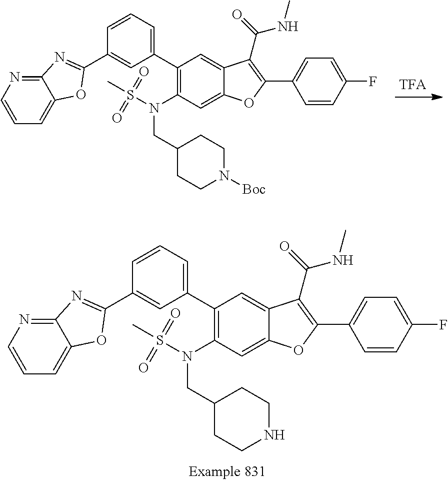

To a 0° C. solution of tert-butyl 4-((N-(2-(4-fluorophenyl)-3-(methylcarbamoyl)-5-(3-(oxazolo[4,5-b]pyridin-2-yl)phenyl)benzofuran-6-yl)methylsulfonamido)methyl)piperidine-1-carboxylate (100 mg, 0.13 mmol, prepared according to the method described in Example 440) in dichloromethane (10 mL) was added TFA (75 mg, 0.66 mmol) was added dropwise. The reaction was allowed to stir at 0° C. for 2 hours, and then was diluted with water and basified with aqueous NaHCO₃ solution. The basified solution was extracted with dichloromethane and the organic extract was washed with brine, dried over Na₂SO₄, filtered and concentrated in vacuo.

The residue obtained was purified using prep-HPLC to provide the title compound (30 mg, 34.6%). ¹H-NMR (CDCl₃, 400 MHz) δ 8.51˜8.56 (m, 2H), 8.32 (d, J=8.0 Hz, 1H), 8.15 (d, J=8.0 Hz, 1H), 7.95˜8.00 (m, 4H), 7.68˜7.72 (m, 2H), 7.48 (d, J=4.0 Hz, 1H), 7.24˜7.29 (m, 2H), 3.38˜3.40 (m, 2H), 3.18˜3.22 (m, 4H), 2.91˜2.97 (m, 4H), 2.75˜2.81 (m, 1H), 2.40˜2.44 (m, 1H), 1.45˜1.67 (m, 1H), 1.30˜1.31 (m, 1H), 1.21˜1.27 (m, 1H), 1.00˜1.04 (m, 2H). MS (M+H)⁺: 654.

### Example 832

### (S)-methyl 1-(44(N-(2-(4-fluorophenyl)-3-(methylcarbamoyl)-5-(3-(oxazolo[4,5-b]pyridin-2-yl)phenyl)benzofuran-6-yl)methylsulfonamido) methyl)piperidin-1-yl)-3-methyl-1-oxobutan-2-ylcarbamate

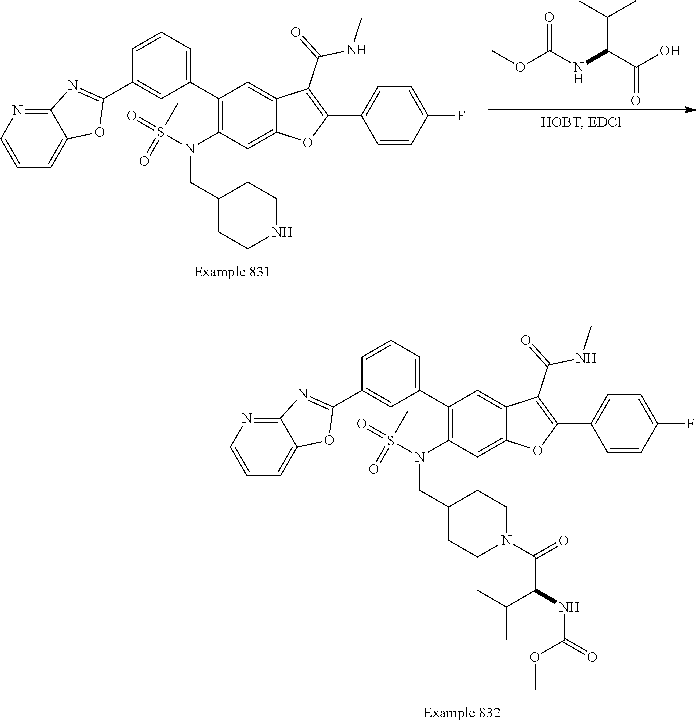

A solution of the compound of Example 831 (86 mg, 0.13 mmol), HOBT (58 mg, 0.43 mmol) and EDCI (84 mg, 0.43 mmol) in dry DMF (3 mL) was allowed to stir at room temperature for 30 minutes. Triethylamine (0.5 mL) and (S)-2-(methoxycarbonylamino)-3-methylbutanoic acid (70 mg, 0.39 mmol) were then added and the reaction was allowed to stir for about 15 hours. The reaction mixture was concentrated in vacuo and the residue obtained was diluted with water and extracted with ethyl acetate. The organic extract was washed with H₂O, brine, dried over Na₂SO₄, filtered and concentrated in vacuo and the residue obtained was purified using prep-HPLC to provide the title compound (52 mg, 49%). ¹H-NMR (CDCl₃, 400 MHz) δ 8.35˜8.54 (m, 2H), 8.30˜8.35 (m, 1H), 8.12˜8.14 (m, 1H), 7.90˜7.97 (m, 4H), 7.67˜7.71 (m, 2H), 7.44 (d, J=4.0 Hz, 1H), 7.22˜7.26 (m, 2H), 3.91˜4.31 (m, 3H), 3.48˜3.55 (m, 3H), 3.16˜3.18 (m, 3H), 2.93 (s, 3H), 2.37˜2.85 (m, 2H), 1.36˜1.80 (m, 4H), 0.81˜0.99 (m, 2H), 0.49˜0.80 (m, 8H). MS (M+H)⁺: 811.

The following compounds of the present invention were made using the method described above and using the appropriate reactants and/or reagents.

| | | | MS |
|---|---|---|---|
| Example | Structure | NMR | (M + H)⁺ |
| 833 | 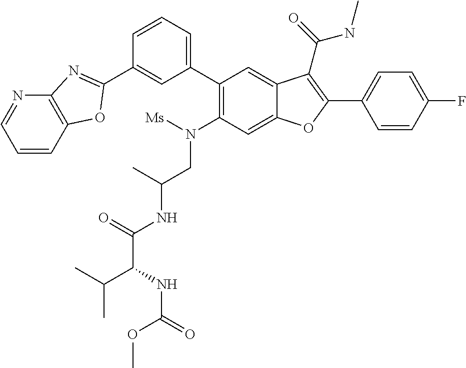 | ¹H-NMR (CDCl₃, 400 MHz) δ 8.54 (s, 1H), 8.30~8.33 (m, 2H), 7.79~7.88 (m, 4H), 7.61~7.63 (m, 3H), 7.45 (s, 1H), 7.26~7.29 (m, 2H), 7.13~7.15 (m, 2H), 6.90~6.97 (m, 1H), 5.02 (s, 1H), 4.00 (s, 1H), 3.73~3.77 (s, 1H), 3.63 (m, 2H), 3.40~3.49 (m, 2H), 3.00 (s, 1H), 2.92 (s, 2H), 2.77 (s, 2H), 2.26 (s, 1H), 1.19 (s, 1H), 0.92~0.95 (m, 4H), 0.81~0.83 (m, 2H), 0.63~0.69 (m, 2H), 0.01 (s, 1H). | 771 |
| 834 | 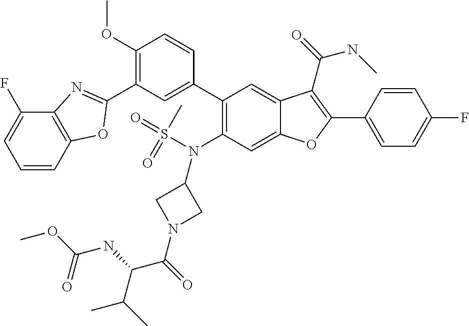 | ¹H-NMR (CDCl₃, 400 MHz) δ 8.15~8.21 (m, 1H), 7.91~7.96 (m, 3H), 7.52~7.67 (m, 2H), 7.44~7.46 (m, 1H), 7.29~7.31 (m, 1H), 7.15~7.22 (m, 3H), 7.04~7.09 (m, 1H), 6.09 (br s, 1H), 5.19~5.31 (m, 1H), 4.71~4.76 (m, 1H), 4.22~4.55 (m, 3H), 4.06 (s, 3H), 3.81~3.98 (m, 2H), 3.51~3.63 (m, 3H), 3.01 (d, J = 4.4 Hz, 3H), 2.73~2.83 (m, 3H), 1.82 (br s, 1H), 0.69~0.90 (m, 6H). | 816 |
| 835 | 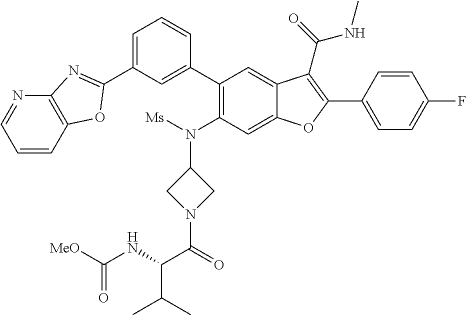 | ¹H-NMR (CDCl₃, 400 MHz) δ 8.58 (d, J = 4.0 Hz, 1H), 8.33~8.39 (m, 2H), 7.91~8.02 (m, 4H), 7.63~7.69 (m, 2H), 7.54 (s, 1H), 7.30~7.34 (m, 1H), 7.19~7.24 (m, 2H), 6.00 (br s, 1H), 5.21~5.35 (m, 1H), 4.57~4.78 (m, 2H), 3.82~4.41 (m, 4H), 3.45~3.64 (m, 3H), 3.01 (d, J = 4.0 Hz, 3H), 2.64~2.77 (m, 3H), 1.83~1.89 (m, 1H), 0.74~0.92 (m, 6H). | 769 |
| 836 | 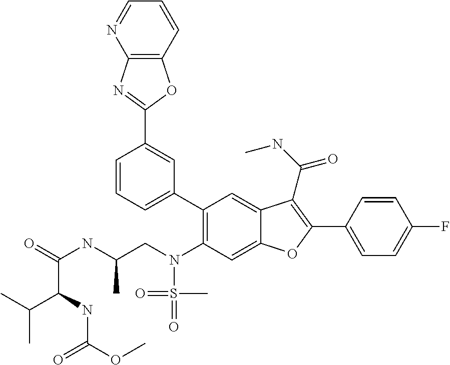 | ¹H-NMR (CDCl₃, 400 MHz) δ 8.54 (s, 1H), 8.30~8.33 (m, 2H), 7.79~7.88 (m, 4H), 7.61~7.63 (m, 3H), 7.45 (s, 1H), 7.26~7.29 (m, 2H), 7.13~7.15 (m, 2H), 6.90~6.97 (m, 1H), 5.02 (s, 1H), 4.00 (s, 1H), 3.73~3.77 (s, 1H), 3.63 (m, 2H), 3.40~3.49 (m, 2H), 3.00 (s, 1H), 2.92 (s, 2H), 2.77 (s, 2H), 2.26 (s, 1H), 1.19 (s, 1H), 0.92~0.95 (m, 4H), 0.81~0.83 (m, 2H), 0.63~0.69 (m, 2H), 0.01 (s, 1H). | 771 |
| 837 | 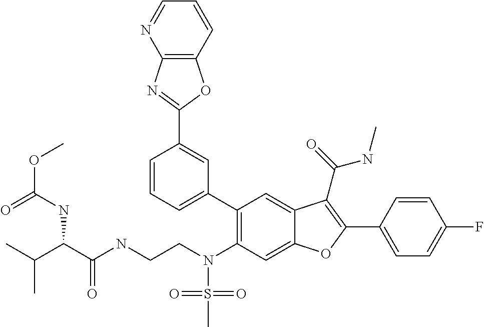 | H-NMR (CDCl₃, 400 MHz) δ 8.56 (d, J = 4.4 Hz, 1H), 8.42 (d, J = 11.6 Hz, 1H), 8.32 (d, J = 8.0 Hz, 1H), 7.87~7.94 (m, 4H), 7.81 (d, J = 7.2 Hz, 1H), 7.62~7.66 (m, 2H), 7.31~7.34 (m, 1H), 7.16~7.20 (m, 2H), 6.60 (d, J = 24.0 Hz, 1H), 6.13~6.18 (m, 1H), 5.20~5.62 (m, 1H), 3.83~3.86 (m, 2H), 3.76~3.78 (m, 1H), 3.66~3.69 (m, 3H), 3.54 (d, J = 14.8 Hz, 1H), 3.39 (d, J = 5.6 Hz, 1H), 3.14 (d, J = 12.0 Hz, 3H), 2.90~2.98 (m, 3H), 2.02~2.07 (m, 1H), 0.84 (d, J = 6.8 Hz, 3H), 0.79 (d, J = 6.4 Hz, 3H). | 757 |

### Example 838

### 6-(N-(3-(cyanomethylamino)propyl)methylsulfonamido)-2-(4-fluorophenyl)-N-methyl-5-(3-(oxazolo[4,5-b]pyridin-2-yl)phenyl)benzofuran-3-carboxamide

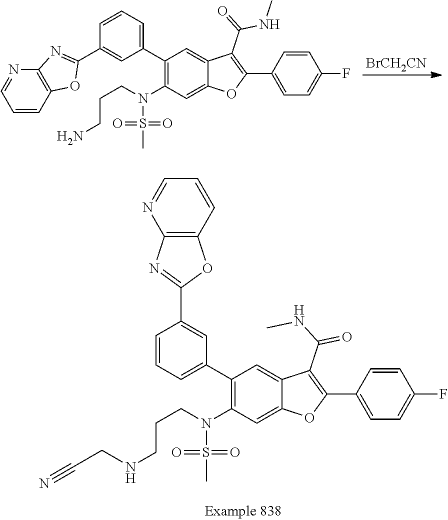

A mixture of 6-(N-(3-aminopropyl)methylsulfonamido)-2-(4-fluorophenyl)-N-methyl-5-(3-(oxazolo[4,5-13]pyridin-2-yl)phenyl)benzofuran-3-carboxamide (100 mg, 0.16 mmol, prepared according to the method described in Example 449), BrCH₂CN (84 mg, 0.71 mmol), K₂CO₃ (97 mg, 0.71 mmol) and KI (27 mg, 0.16 mmol) in dry DMF (2.0 mL) was heated to 100° C. and allowed to stir at this temperature for about 15 hours. The reaction mixture was cooled to room temperature and filtered, then the filtrate was washed with H₂O, brine, dried over Na₂SO₄, filtered and concentrated in vacuo. The residue obtained was purified using prep-HPLC to provide the title compound (21 mg, 19.8%). ¹H-NMR (CDCl₃, 400 MHz) δ 8.47˜8.49 (m, 1H), 8.32 (s, 1H), 8.23 (d, J=7.2 Hz, 1H), 7.86˜7.88 (m, 2H), 7.79˜7.83 (m, 2H), 7.71˜7.73 (m, 1H), 7.55˜7.57 (m, 2H) 7.22=7.26 (m, 1H), 7.10˜7.14 (m, 2H), 6.08 (d, J=4.4 Hz, 1H), 3.39 (s, 4H), 2.92 (d, J=5.2 Hz, 3H), 2.84 (s, 3H), 2.38˜2.42 (m, 2H), 1.53 (s, 3H). MS (M+H)⁺: 653.

The following compound of the present invention was made using the method described above and using the appropriate reactants and/or reagents.

| | | | MS |
|---|---|---|---|
| Example | Structure | NMR | (M + H)⁺ |
| 839 | 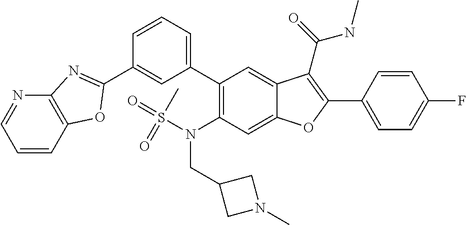 | ¹H-NMR (MeOD, 400 MHz) δ 8.58 (s, 1H), 8.28 (d, J = 7.6 Hz, 1H), 8.21 (d, J = 8.0 Hz, 1H), 7.96~8.03 (m, 4H), 7.85 (d, J = 1.6 Hz, 1H), 7.83 (d, J = 1.6 Hz, 2H), 7.71~7.78 (m, 1H), 7.61 (d, J = 1.6 Hz, 3H), 7.54 (t, J = 7.6 Hz, 1H), 4.84 (s, 1H), 4.48~4.54 (m, 1H), 3.10~3.15 (m, 1H), 2.87~2.93 (m, 1H), 2.17 (s, 3H), 1.31 (d, J = 6.4 Hz, 3H). | 640 |

### Example 840

### 34N-(2-(4-fluorophenyl)=3-(methylcarbamoyl)-543-(oxazolo[4,5-b]pyridin-2-yl)phenyl)benzofuran-6-yl)methylsulfonamido) propanoic acid

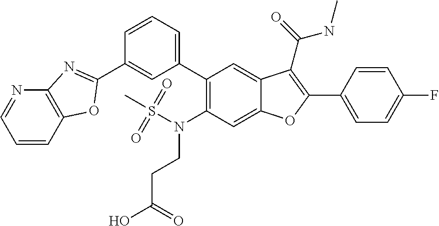

### Step 1—Synthesis of 2-(4-fluorophenyl)-N-methyl-5-(3-(oxazolo[4,5-b]pyridin-2-yl)phenyl)-6-(N-(3-oxopropyl)methylsulfonamido)benzofuran-3-carboxamide

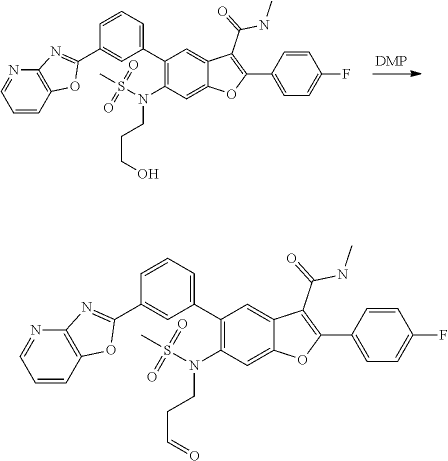

A solution of 2-(4-fluorophenyl)-6-(N-(3-hydroxypropyl)methylsulfonamido)-N-methyl-5-(3-(oxazolo[4,5-b]pyridin-2-yl)phenyl)benzofuran-3-carboxamide (350 mg, 0.57 mmol, prepared according to the method described in Example 440) and DMP (1.2 g, 2.8 mmol) in dichloromethane (10 mL) was allowed to stir at room temperature for 2 hours under N₂ atmosphere. The reaction was quenched with saturated aqueous NaHCO₃ and excess Na₂S₂O₄ and stirred until all solids were dissolved. The solution was then extracted with dichloromethane and the organic extract was washed with brine, dried over Na₂SO₄, filtered and concentrated in vacuo to provide 2-(4-fluorophenyl)-N-methyl-5-(3-(oxazolo[4,5-b]pyridin-2-yl)phenyl)-6-(N-(3-oxopropyl)methylsulfonamido)benzofuran-3-carboxamide (315 mg, 90.2%), which was used without further purification.

### Step 2—Synthesis of 3-(N-(2-(4-fluorophenyl)-3-(methylcarbamoyl)-5-(3-(oxazolo[4,5-b]pyridin-2-yl)phenyl)benzofuran-6-yl)methylsulfonamido)propanoic acid

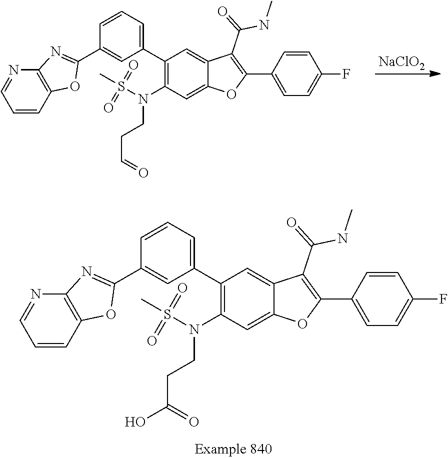

To a 0° C. solution of 2-(4-fluorophenyl)-N-methyl-5-(3-(oxazolo[4,5-b]pyridin-2-yl)phenyl)-6-(N-(3-oxopropyl)methylsulfonamido)benzofuran-3-carboxamide (300 mg, 0.49 mmol), NaH₂PO₄ (180 mg, 4 mmol) and NH₂SO₃H (72 mg, 0.75 mmol) in dioxane (5 mL) was added a solution of NaClO₂ (180 mg, 2 mmol) in H₂O (2 mL) dropwise. The reaction was allowed to stir for 10 minutes at 0° C., then the cold bath was removed and the reaction mixture was warmed up to room temperature and stirred for another 15 minutes. The reaction was diluted with water, extracted with dichloromethane and the organic extract was washed with brine, dried over Na₂SO₄, filtered and concentrated in vacuo to provide the title compound (210 mg, 67.8%). ¹H-NMR (DMSO, 400 MHz) δ 8.57 (s, 1H), 8.28 (s, 1H), 8.17˜8.19 (m, 1H), 7.81˜7.87 (m, 2H), 7.56˜7.58 (m, 3H), 7.25˜7.26 (m, 2H), 7.18˜7.20 (m, 1H), 7.11˜7.15 (m, 2H), 5.98 (s, 1H), 3.64˜3.67 (m, 2H), 2.92˜2.93 (m, 3H), 2.81 (s, 3H), 2.51˜2.52 (m, 2H). MS (M+H)⁺: 629.

### Example 841

### (S)-methyl 2-(3-(N-(2-(4-fluorophenyl)-3-(methylcarbamoyl)-5-(3-(oxazolo[4,5-b]pyridin-2-yl)phenyl)benzofuran-6-yl)methylsulfonamido) propanamido)-3-methylbutanoate

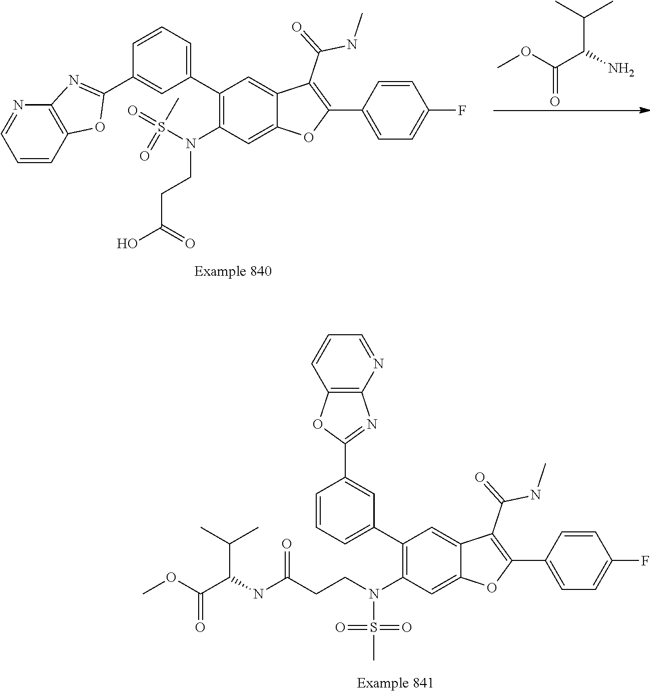

The compound of Example 840 was converted to the title compound (30 mg, 36%) using the method described in Example 411, Step 5. ¹H-NMR (CDCl₃, 400 MHz) δ 8.57 (s, 1H), 8.38 (s, 1H), 8.21 (m, J=6.4 Hz, 1H), 8.06 (s, 1H), 7.88 (s, 2H), 7.78˜7.80 (m, 1H), 7.42˜7.59 (m, 3H), 7.11˜7.15 (m, 3H), 6.21 (s, 2H), 4.29 (s, 1H), 3.57 (d, J=4.4 Hz, 3H), 3.25˜3.32 (m, 1H), 2.92˜2.98 (m, 6H), 2.36 (s, 2H), 1.98˜2.01 (m, 2H), 1.18 (s, 6H). MS (M+H)⁺: 742.

The following compound of the present invention was made using the method described above and using the appropriate reactants and/or reagents.

| | | | MS |
|---|---|---|---|
| Example | Structure | NMR | (M + H)⁺ |
| 842 | 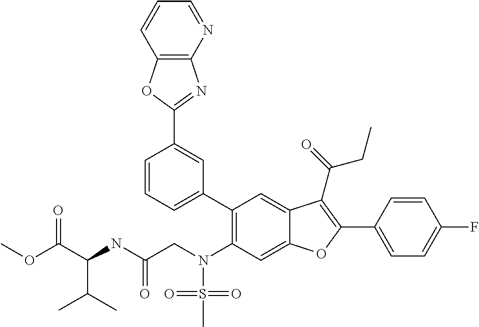 | ¹H-NMR (CDCl₃, 400 MHz) δ 8.52 (s, 1H), 8.38 (s, 1H), 8.21 (s, 1H), 7.93~7.99 (m, 2H), 7.83~7.86 (m, 2H), 7.73 (d, J = 5.6 Hz, 1H), 7.48 (d, J = 7.6 Hz, 1H), 7.30 (s, 1H), 7.17~7.21 (m, 2H), 6.98 (s, 1H), 6.16 (s, 1H), 4.46 (s, 1H), 3.67 (s, 3H), 3.28 (s, 3H), 2.99 (d, J = 4.8 Hz, 3H), 2.06~2.09 (m, 1H), 1.97 (s, 3H), 0.82 (d, J = 4.8 Hz, 6H). | 728 |

### Examples 843 and 844

### 34N-(5-(3-(4-fluorobenzo[d]oxazol-2-yl)-4-methoxyphenyl)-2-(4-fluorophenyl)-3-(methylcarbamoyl)benzofuran-6-yl)methylsulfonamido)propylphosphonic acid

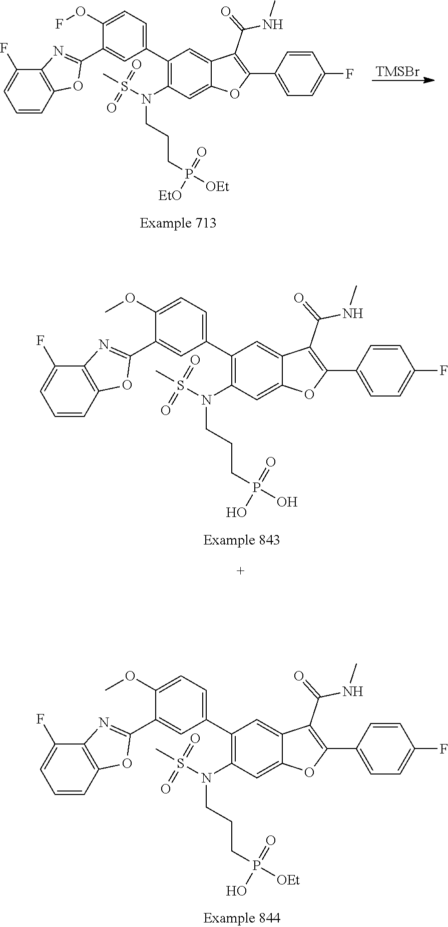

To a solution of the compound of Example 713 (100 mg, 0.13 mmol) in CH₃CN (2 mL) was added TMSBr (2.0 g, 9.2 mmol). The reaction was allowed to stir for 16 hours, then was quenched with water and extracted with CH₂Cl₂. The organic layer was washed with brine, dried over Na₂SO₄, filtered and concentrated in vacuo and the residue obtained was purified prep-HPLC to provide Example 843 (20 mg, 20%) and Example 844 (10 mg, 10%).

Example 843: ¹H-NMR (CDCl₃, 400 MHz) δ 7.98 (s, 1H), 7.35˜7.59 (m, 5H), 7.19˜7.23 (s, 1H), 7.12 (s, 1H), 6.86˜6.88 (m, 4H), 3.77 (s, 3H), 3.16˜3.33 (m, 2H), 2.65˜2.85 (m, 6H), 1.18˜1.51 (m, 4H). MS (M+H)⁺: 726.

### Example 843

### ¹H-NMR (CDCl₃, 400 MHz) δ 7.92 (s, 1H), 7.84˜7.87 (m, 3H), 7.72 (s, 1H), 7.52 (s, 1H), 7.39 (s, 1H), 7.28˜7.29 (m, 1H), 7.03˜7.11 (m, 4H), 6.15 (s, 1H), 3.94 (s, 5H), 3.41 (s, 2H), 3.00 (s, 3H), 2.90 (d, J=4.4 Hz, 3H), 1.31˜1.61 (m, 4H), 1.14˜1.16 (m, 3H). MS (M+H)⁺: 754.

### Example 845

### 5-(3-(1H-indol-2-yl)-4-methoxyphenyl)-2-(4-fluorophenyl)-N-methyl-6-(N-methylmethylsulfonamido)-N-(pyridin-3-yl)benzofuran-3-carboxamide

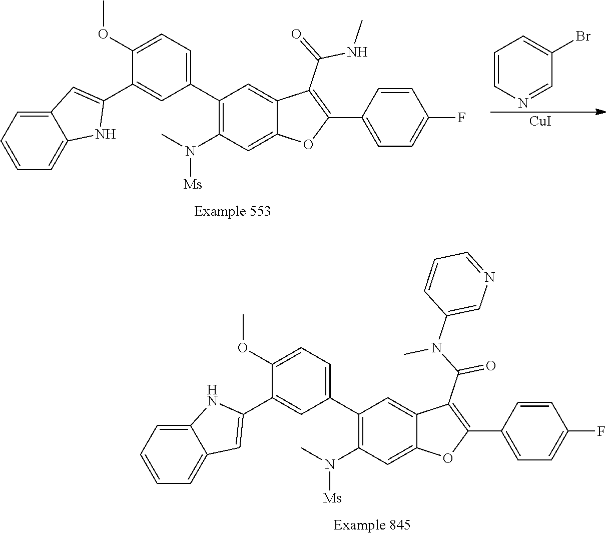

A solution of the compound of Example 553 (100 mg, 0.17 mmol), 3-Bromo-pyridine (40 mg, 0.25 mmol) and CuI (3 mg) in toluene (1.5 mL) was put under nitrogen atmosphere and heated to 110° C. The reaction was stirred and monitored using TLC. When the starting material was consumed, the reaction mixture was cooled to RT and the reaction mixture was concentrated in vacuo. The residue obtained was purified using Prep-HPLC to provide the title compound (30 mg, 26.5%). ¹H-NMR (CDCl₃, 400 MHz) δ 8.00 (t, J=8.0 Hz, 2H), 7.83 (s, 1H), 7.68 (d, J=8.0 Hz, 1H), 7.60˜7.52 (m, 1H), 7.36 (d, J=8.0 Hz, 4H), 7.31 (d, J=7.2 Hz, 2H), 7.20 (t, J=8.4 Hz, 3H), 6.75 (s, 1H), 6.22 (s, 1H), 5.74 (d, J=11.6 Hz, 1H), 5.57 (s, 1H), 4.83 (s, 2H), 3.19 (s, 3H), 3.03 (d, J=4.4 Hz, 3H), 2.72 (s, 3H). MS (M+H)⁺: 675.

The following compounds of the present invention were made using the method described above and using the appropriate reactants and/or reagents.

| | | | MS |
|---|---|---|---|
| Example | Structure | NMR | (M + H)⁺ |
| 846 | 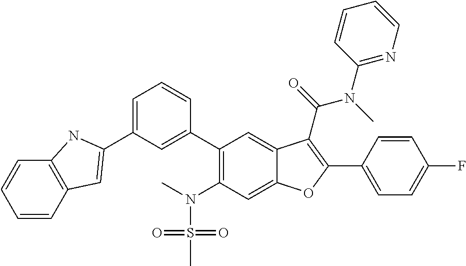 | ¹H-NMR (CDCl₃, 400 MHz) δ 9.07 (s, 1H), 8.17 (s, 1H), 7.91 (s, 1H), 7.73~7.74 (m, 2H), 7.62~7.66 (m, 3H), 7.49~7.54 (m, 2H), 7.42~7.45 (m, 2H), 7.35~7.37 (m, 1H), 7.17~7.20 (m, 2H), 7.09~7.14 (m, 3H), 6.93~6.96 (m, 2H), 3.56 (s, 3H), 2.95~2.97 (m, 6H). | 645 |
| 847 | 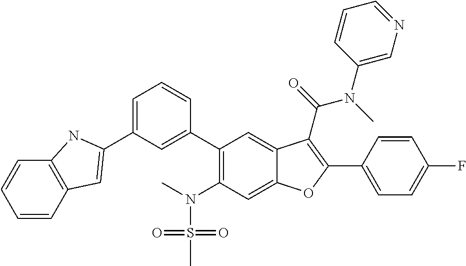 | ¹H-NMR (CDCl₃, 400 MHz) δ 10.28 (s, 1H), 8.35~8.41 (m, 2H), 7.77 (d, J = 7.2 Hz, 3H), 7.64 (d, J = 7.6 Hz, 2H), 7.44~7.51 (m, 4H), 7.34 (d, J = 8.0 Hz, 1H), 7.08~7.19 (m, 5H), 6.98 (s, 1H), 6.89 (s, 1H), 3.56 (s, 3H), 3.01 (s, 3H), 2.71 (s, 3H). | 645 |
| 848 | 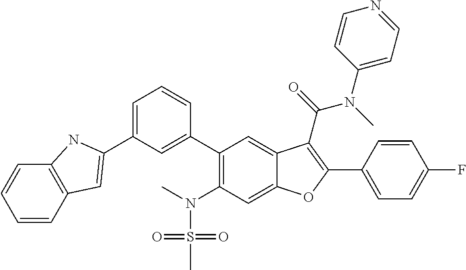 | ¹H-NMR (CDCl₃, 400 MHz) δ 10.28 (s, 1H), 8.35~8.41 (m, 2H), 7.77 (d, J = 7.2 Hz, 3H), 7.64 (d, J = 7.6 Hz, 2H), 7.44~7.51 (m, 4H), 7.34 (d, J = 8.0 Hz, 1H), 7.08~7.19 (m, 5H), 6.98 (s, 1H), 6.89 (s, 1H), 3.56 (s, 3H), 3.01 (s, 3H), 2.71 (s, 3H). | 645 |
| 849 | 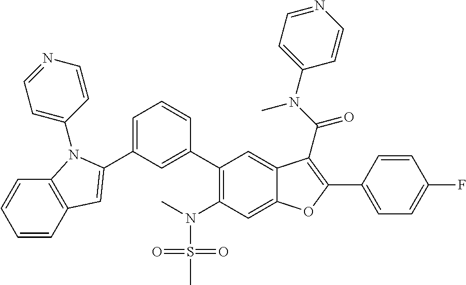 | ¹H-NMR (CDCl₃, 400 MHz) δ 8.81 (s, 2H), 8.53 (s, 2H), 7.68~7.75 (m, 3H), 7.57~7.59 (m, 3H), 7.46~7.50 (m, 3H), 7.29~7.42 (m, 7H), 7.14~7.18 (t, 2H), 6.99 (s, 1H), 3.50 (s, 3H), 3.25 (s, 3H), 2.91 (s, 3H). | 722 |
| 850 | 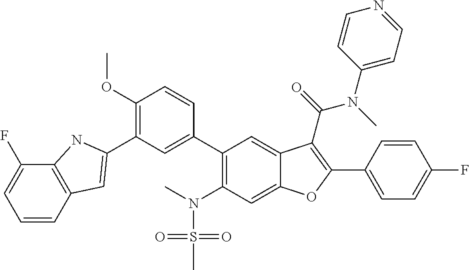 | ¹H-NMR (CDCl₃, 400 MHz) δ 8.36~8.37 (m, 2H), 8.16~8.17 (m, 1H), 7.63~7.66 (m, 2H), 7.56 (s, 1H), 7.46~7.47 (m, 2H), 7.36~7.38 (m, 2H), 7.22~7.28 (m, 6H), 4.02 (s, 3H), 3.44 (s, 3H), 3.09 (s, 3H), 2.75 (s, 3H). | 695 |

### Example 851

### 5-(3-(1H-indol-2-yl)phenyl)-2-(4-fluorophenyl)-6-(N-methylmethylsulfonamido)-N-(pyridin-3-yl)benzofuran-3-carboxamide

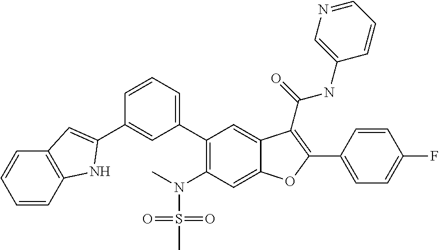

### Step 1—Synthesis of ethyl 5-(3-(1H-indol-2-yl)phenyl)-2-(4-fluorophenyl)-6-(N-methylmethylsulfonamido)benzofuran-3-carboxylate

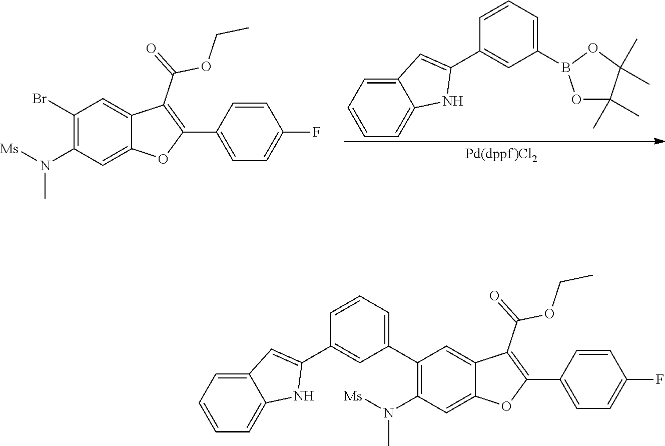

A mixture of ethyl 5-bromo-2-(4-fluorophenyl)-6-(N-methylmethylsulfonamido)benzofuran-3-carboxylate (884 mg, 1.9 mmol, prepared from Compound 411E with MeI according to the method described in Example 411, step 7), 2-(3-(4,4,5,5-tetramethyl-1,3,2-dioxaborolan-2-yl)phenyl)-1H-indole (746 mg, 2.3 mmol, prepared from corresponding bromide), K₃PO₄ (1.03 g, 188 mmol) and Pd(dppf)Cl₂ (142 mg, 0.19 mmol) in DMF (10 mL) was heated to 100° C. and allowed to stir at this temperature for 8 hour under N₂ atmosphere. The reaction was poured into ice water, the resulting solution was filtered and the collected solid was washed with water and dried to provide ethyl 5-(3-(1H-indol-2-yl)phenyl)-2-(4-fluorophenyl)-6-(N-methylmethylsulfonamido) benzofuran-3-carboxylate (0.88 g, 79% yield), which was used without further purification. MS (M+H)⁺: 583.

### Step 2—Synthesis of 5-(3-(1H-indol-2-yl)phenyl)-2-(4-fluorophenyl)-6-(N-methylmethylsulfonamido)benzofuran-3-carboxylic acid

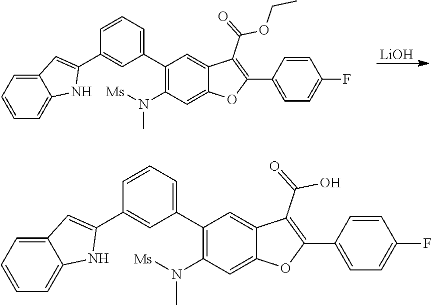

A mixture of ethyl 5-(3-(1H-indol-2-yl)phenyl)-2-(4-fluorophenyl)-6-(N-methylmethylsulfonamido)benzofuran-3-carboxylate (870 mg, 1.53 mmol), and LiOH (320 mg, 7.65 mmol) in 1,4-dioxane/water (1/1, 40 mL) was heated to 100° C. and allowed to stir at this temperature for 2 hours. The reaction mixture was cooled to RT, concentrated in vacuo and the resulting residue was diluted by water. The resulting solution was adjusted to pH 3 using 1N HCl and the acidified solution was filtered. The collected solid was washed with water and dried to provide 5-(3-(1H-indol-2-yl)phenyl)-2-(4-fluorophenyl)-6-(N-methylmethylsulfonamido) benzofuran-3-carboxylic acid (0.8 g, 94%). ¹H-NMR (DMSO, 400 MHz) δ 13.38 (s, 1H), 11.58 (s, 1H), 8.13˜8.16 (m, 2H), 8.04 (d, J=9.2 Hz, 2H), 7.94 (s, 1H), 7.89 (d, J=7.6 Hz, 1H), 7.54 (t, J=7.2 Hz, 2H), 7.38˜7.46 (m, 4H), 7.10 (t, J=8.0 Hz, 1H), 6.99 (t, J=8.0 Hz, 1H), 6.95 (s, 1H), 3.14 (s, 3H), 2.94 (s, 3H). MS (M+H)⁺: 555.

### Step 3—Synthesis of 5-(3-(1H-indol-2-yl)phenyl)-2-(4-fluorophenyl)-6-(N-methylmethylsulfonamido)-N-(pyridin-3-yl)benzofuran-3-carboxamide

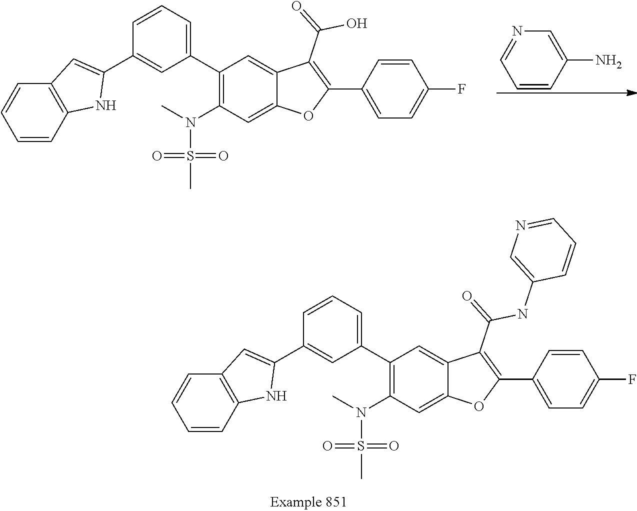

A mixture of 5-(3-(1H-indol-2-yl)phenyl)-2-(4-fluorophenyl)-6-(N-methylmethylsulfonamido)benzofuran-3-carboxylic acid (80 mg, 0.14 mmol), pyridin-3-amine (17 mg, 0.17 mmol), PyBOP (80 mg, 0.17 mmol), and DIPEA (27 mg, 0.21 mmol) in DMF (1 mL) was allowed to stir for 12 hours. Water was added, then the resulting solution was extracted with ethyl acetate and the organic extract was washed with brine, dried over Na₂SO₄, filtered and concentrated in vacuo. The residue obtained was purified using prep-HPLC to provide the title compound (30 mg, 33%). ¹H-NMR (CDCl₃, 400 MHz) δ 9.67 (s, 1H), 8.78˜8.92 (m, 2H), 8.28 (s, 1H), 8.08 (s, 1H), 7.74 (s, 3H), 7.64˜7.67 (m, 3H), 7.47˜7.49 (m, 1H), 7.36 (s, 2H), 7.14˜7.22 (m, 6H), 6.82 (s, 1H), 2.91 (s, 6H). MS (M+H)⁺: 631.

The following compound of the present invention was made using the method described above and using the appropriate reactants and/or reagents.

| | | | MS |
|---|---|---|---|
| Example | Structure | NMR | (M + H)⁺ |
| 852 | 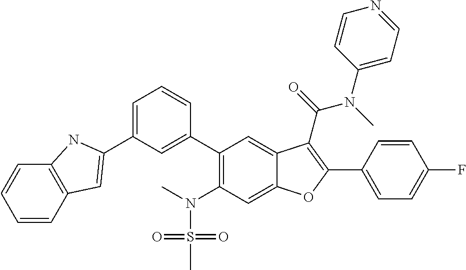 | ¹H-NMR (CDCl₃, 400 MHz) δ 9.45 (s, 1H), 8.05 (s, 2H), 7.73~7.81 (m, 4H), 7.66 (s, 1H), 7.46~7.51 (m, 2H), 7.38 (m, 1H), 7.24~7.30 (m, 2H), 7.15~7.17 (m, 2H), 7.04~7.10 (m, 3H), 6.96~6.99 (m, 2H), 6.67 (s, 1H), 2.90 (s, 3H), 2.70 (s, 3H). | 631 |

### Example 853

### 5-(3-(4-fluorobenzoldloxazol-2-O-4-methoxyphenyl)-2-(4-fluorophenyl)-N-methyl-6-(2-oxooxazolidin-3-yl)benzofuran-3-carboxamide

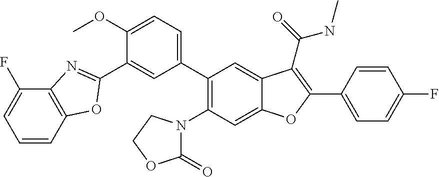

### Step 1—Synthesis of ethyl 5-bromo-2-(4-fluorophenyl)-6-(2-oxooxazolidin-3-yl) benzofuran-3-carboxylate

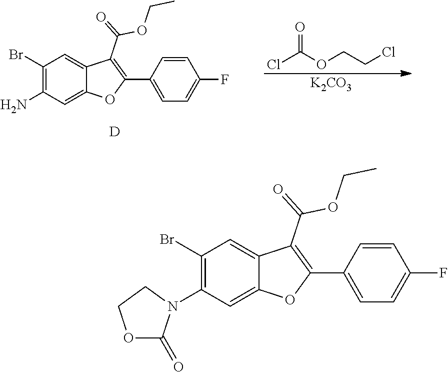

2-Chloroethyl chloroformate (0.38 g, 2.6 mmol) and K₂CO₃ (0.75 g, 7.2 mmol) were added to a solution of Compound 411D (0.5 g, 1.3 mmol, prepared described in Example 1, step 3) in MeCN (10 mL) under N₂ atmosphere. The reaction was heated to reflux (80° C.) and allowed to stir at this temperature for about 15 hours. The reaction mixture was then filtered and concentrated in vacuo, and the residue obtained was purified using column chromatography (petroleum ether:EtOAc=4:1) to provide ethyl 5-bromo-2-(4-fluorophenyl)-6-(2-oxooxazolidin-3-yl)benzofuran-3-carboxylate (350 mg, 59%). ¹H-NMR (CDCl₃, 400 MHz) δ 8.35 (s, 1H), 8.04˜8.08 (m, 2H), 7.61 (s, 1H), 7.17˜7.21 (m, 2H), 4.59 (t, J=8.0 Hz, 2H), 4.43 (q, J=7.2 Hz, 2H), 4.08 (t, J=8.0 Hz, 2H), 1.42 (t, J=7.2 Hz, 3H). MS (M+H)⁺: 448/450.

### Step 2—Synthesis of 5-bromo-2-(4-fluorophenyl)-6-(2-oxooxazolidin-3-yl)benzofuran-3-carboxylic acid

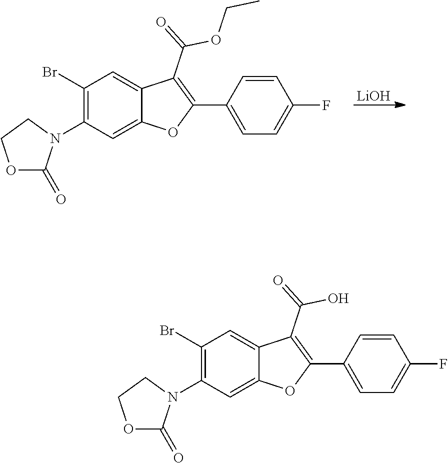

To a solution of ethyl 5-bromo-2-(4-fluorophenyl)-6-(2-oxooxazolidin-3-yl)benzofuran-3-carboxylate (350 mg, 0.78 mmol) in dioxane (6 mL) and water (6 mL) was added LiOH (187 mg, 7.81 mmol). The reaction was heated to reflux and allowed to stir at this temperature for 3 hours. The reaction mixture was concentrated in vacuo and the resulting residue was diluted with water. The solution was acidified to pH=6-7 using 1 N HCl, and extracted with EtOAc. The organic extract was washed with brine, dried over Na₂SO₄, filtered and concentrated in vacuo to provide 5-bromo-2-(4-fluorophenyl)-6-(2-oxooxazolidin-3-yl)benzofuran-3-carboxylic acid (300 mg, 92%), which was used without further purification.

### Step 3—Synthesis of 5-bromo-2-(4-fluorophenyl)-N-methyl-6-(2-oxooxazolidin-3-yl)benzofuran-3-carboxamide

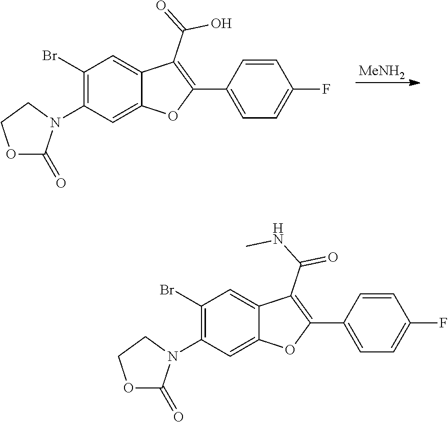

5-bromo-2-(4-fluorophenyl)-6-(2-oxooxazolidin-3-yl)benzofuran-3-carboxylic acid (300 mg, 0.72 mmol), HOBT (145 mg, 1.07 mmol) and EDCI (166 mg, 1.07 mmol) were taken up in dry DMF (8 mL). The resulting reaction was allowed to stir for 30 minutes, then methanamine HCl salt (44 mg, 1.43 mmol) and Et₃N (1 mL) were added. The reaction was then allowed to stir for about 15 hours, then the reaction mixture was diluted with water and extracted with EtOAc. The organic extract was washed with brine, dried over Na₂SO₄, filtered and concentrated in vacuo and the residue obtained was purified using prep-TLC (petroleum ether:EtOAc=2:1) to provide pure 5-bromo-2-(4-fluorophenyl)-N-methyl-6-(2-oxooxazolidin-3-yl)benzofuran-3-carboxamide (200 mg, 66%). ¹H-NMR (CDCl₃, 400 MHz) δ 7.99 (s, 1H), 7.81 (br s, 2H), 7.46 (s, 1H), 7.12˜7.16 (m, 2H), 6.29 (br s, 1H), 4.55 (t, J=8.0 Hz, 2H), 4.03 (t, J=8.0 Hz, 2H), 2.92 (d, J=4.8 Hz, 3H). MS (M+H)⁺: 433/435.

### Step 4—Synthesis of 5-(3-(4-fluorobenzo[d]oxazol-2-yl)-4-methoxyphenyl)-2-(4-fluorophenyl)-N-methyl-6-(2-oxooxazolidin-3-yl)benzofuran-3-carboxamide

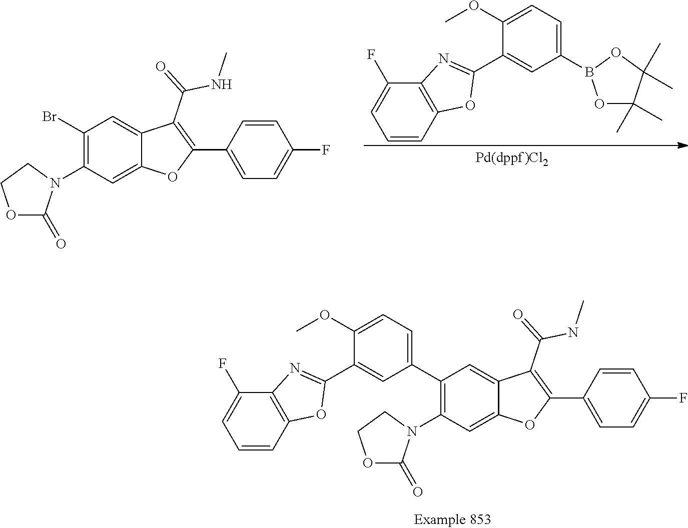

To a mixture of 5-bromo-2-(4-fluorophenyl)-N-methyl-6-(2-oxooxazolidin-3-yl)benzofuran-3-carboxamide (50 mg, 0.12 mmol), 4-fluoro-2-(2-methoxy-5-(4,4,5,5-tetramethyl-1,3,2-dioxaborolan-2-yl)phenyl)benzo[d]oxazole (51 mg, 0.14 mmol) and K₃PO₄.3H₂O (60 mg, 0.23 mmol) in 1,4-dioxane (2 mL), was Pd(dppf)Cl₂(5 mg). The reaction was put under N₂ atmosphere, heated to 100° C. and allowed to stir at this temperature for about 15 hours. The reaction was then cooled to room temperature and concentrated in vacuo and the residue obtained was diluted with water, and extracted with EtOAc. The organic extract was washed with brine, dried over Na₂SO₄, filtered and concentrated in vacuo and the residue obtained was purified using prep-HPLC to provide the title compound (50 mg, 82%). ¹H-NMR (CDCl₃, 400 MHz) δ 8.11 (d, J=2.0 Hz, 1H), 7.79˜7.82 (m, 2H), 7.71 (s, 1H), 7.50˜7.53 (m, 2H), 7.40 (d, J=8.0 Hz, 1H), 7.23˜7.32 (m, 1H), 7.15 (d, J=8.4 Hz, 1H), 7.02˜7.07 (m, 3H), 6.84 (br s, 1H), 4.23 (t, J=8.0 Hz, 2H), 4.09 (s, 3H), 3.47 (t, J=8.0 Hz, 2H), 3.11 (d, J=4.4 Hz, 3H). MS (M+H)⁺: 596.

The following compound of the present invention was made using the method described above and using the appropriate reactants and/or reagents.

| | | | MS |
|---|---|---|---|
| Example | Structure | NMR | (M + H)⁺ |
| 854 | 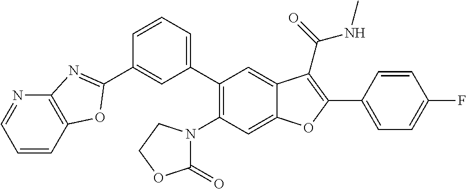 | ¹H-NMR (CDCl₃, 400 MHz) δ 8.58 (br s, 1H), 8.34~8.38 (m, 2H), 7.89~7.96 (m, 4H), 7.60~7.70 (m, 3H), 7.31~7.34 (m, 1H), 7.17~7.22 (m, 2H), 6.10 (br s, 1H), 4.24 (t, J = 8.0 Hz, 2H), 3.55 (t, J = 8.0 Hz, 2H), 3.01 (d, J = 4.8 Hz, 3H). | 549 |

### Examples 855-880

The following compounds of the present invention were made using the methods described in the Examples above, and using the appropriate reactants and/or reagents.

| | | MS |
|---|---|---|
| Example | Structure | (M + H)⁺ |
| 855 | 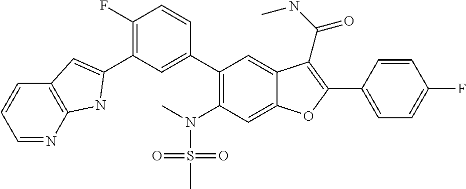 | 587 |
| 856 | 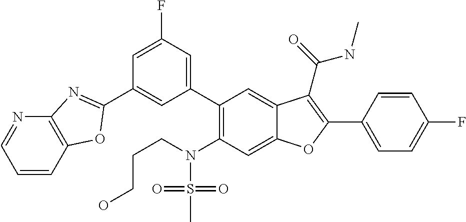 | 633 |
| 857 | 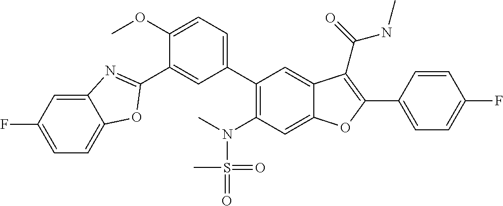 | 618 |
| 858 | 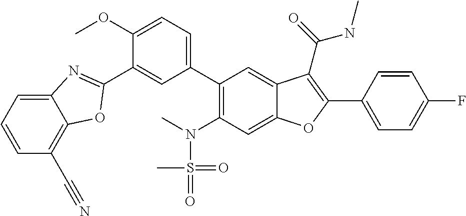 | 625 |
| 859 | 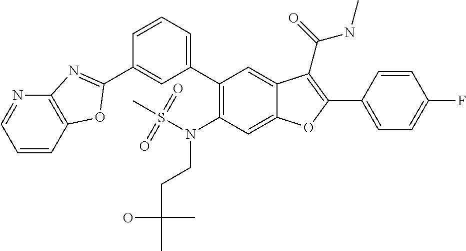 | 643 |
| 860 | 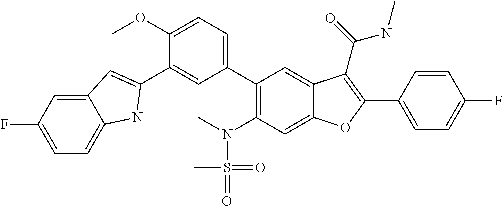 | NA |
| 861 | 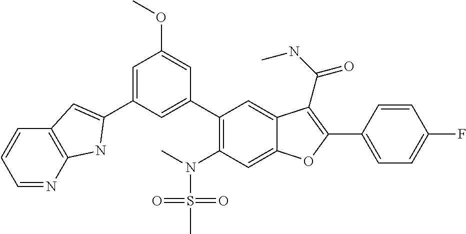 | 599 |
| 862 | 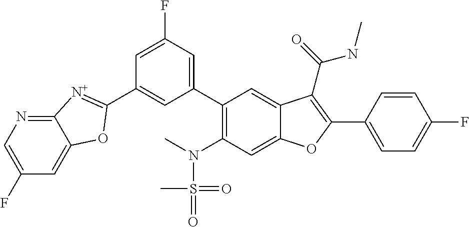 | NA |
| 863 | 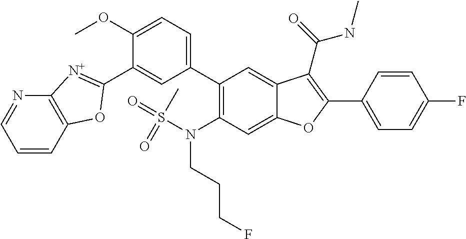 | 647 |
| 864 | 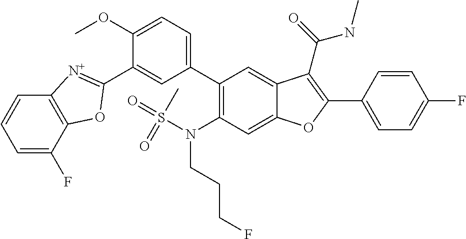 | 644 |
| 865 | 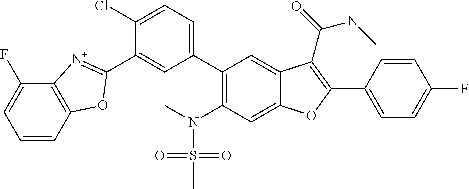 | 622 |
| 866 | 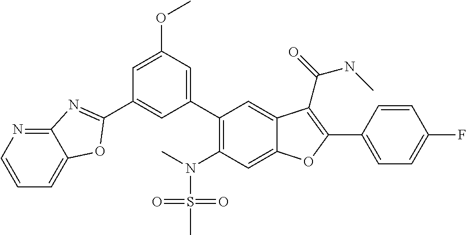 | NA |
| 867 | 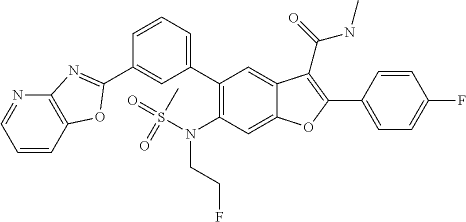 | 603 |
| 868 | 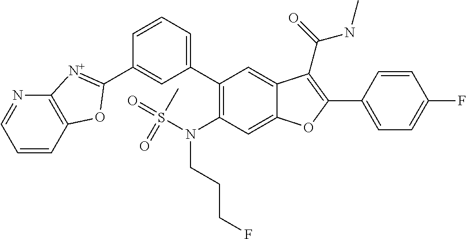 | 617 |
| 869 | 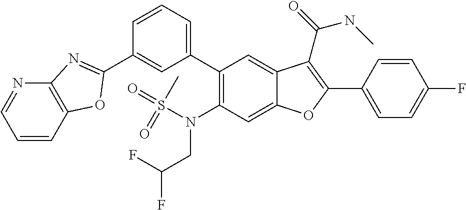 | 621 |
| 870 | 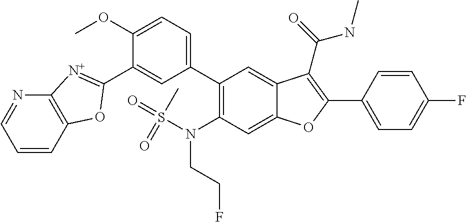 | 633 |
| 871 | 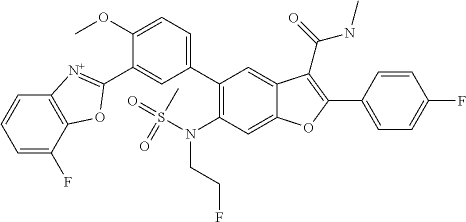 | 650 |
| 872 | 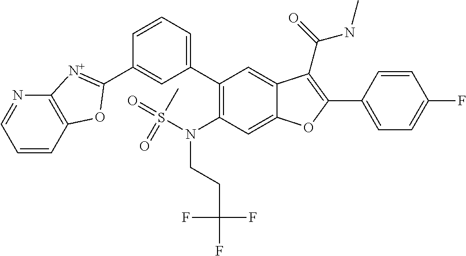 | 653 |
| 873 | 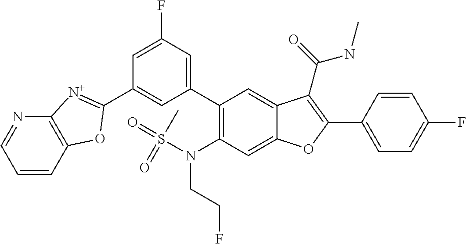 | 621 |
| 874 | 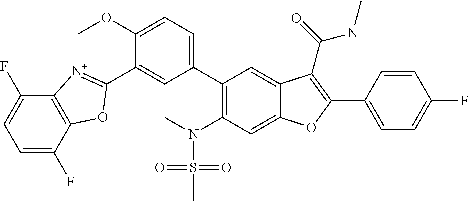 | 636 |
| 875 | 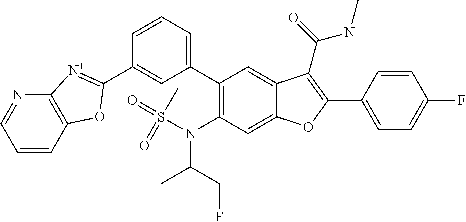 | 617 |
| 876 | 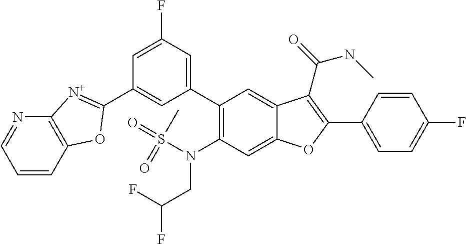 | 639 |
| 877 | 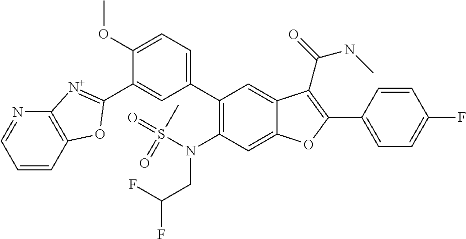 | 651 |
| 878 | 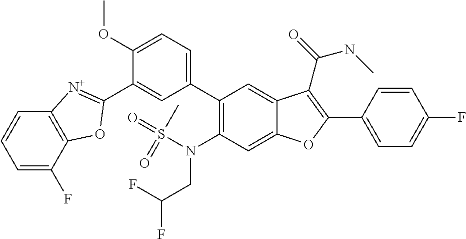 | 668 |
| 879 | 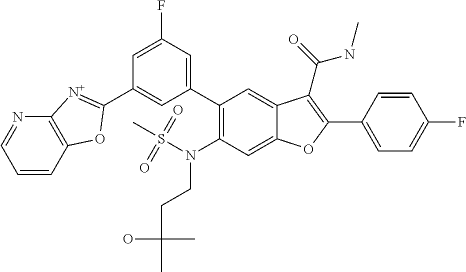 | 662 |
| 880 | 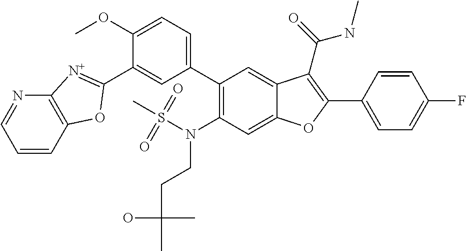 | 673 |

NA = not available

### Example 881

### Measuring Compound Inhibitory Potency

Measurement of inhibition by compounds was performed using the HCV replicon system. Several different replicons encoding different HCV genotypes or mutations were used. In addition, potency measurements were made using different formats of the replicon assay, including different ways of measurements and different plating formats. See Jan M. Vrolijk et al., A replicons-based bioassay for the measurement of interferons in patients with chronic hepatitis C, 110 J. VIROLOGICAL METHODS 201 (2003); Steven S. Carroll et al., Inhibition of Hepatitis C Virus RNA Replication by 2′-Modified Nucleoside Analogs, 278(14) J. BIOLOGICAL CHEMISTRY 11979 (2003). However, the underlying principles are common to all of these determinations, and are outlined below.

Stable neomycin phosphotransferase encoding replicons-harboring cell lines were used, so all cell lines were maintained under G418 selection prior to the assay. Potency was determined using a cell ELISA assay with an antibody to the replicons encoded NS3/4a protease. See Caterina Trozzi et al., In Vitro Selection and Characterization of Hepatitis C Virus Serine Protease Variants Resistant to an Active-Site Peptide Inhibitor, 77(6) J. Virol. 3669 (2003). To initiate an assay, replicon cells were plated in the presence of a dilution series of test compound in the absence of G418. Typically, the assays were performed in a 96-well plate formate for manual operation, or a 384-well plate format for automated assay. Replicon cells and compound were incubated for 96 hours. At the end of the assay, cells were washed free of media and compound, and the cells were then lysed. RNA was quantified indirectly through detection of replicon-encoded NS3/4A protein levels, through an ELISA-based assay with an antibody specific for NS3/4A. IC₅₀ determinations were calculated as a percentage of a DMSO control by fitting the data to a four-parameter fit function and the data obtained is provided in the table below.

The activity tables provided below illustrate the observed activity of selected compounds of the present invention:

| | Replicon 1b |
|---|---|
| Example | IC₅₀ (nM) |
| 1 | 120 |
| 2 | 114 |
| 3 | 80 |
| 4 | 703 |
| 5 | 47 |
| 6 | 118 |
| 7 | ND |
| 8 | 276 |
| 9 | 99 |
| 10 | 221 |
| 11 | ND |
| 12 | ND |
| 13 | 84 |
| 14 | ND |
| 16 | 68 |
| 17 | 48 |
| 18 | 41 |
| 19 | 227 |
| 20 | 66 |
| 21 | 115 |
| 22 | 55 |
| 23 | 374 |
| 24 | 182 |
| 25 | 166 |
| 27 | 384 |
| 28 | 46 |
| 29 | 412 |
| 30 | 40 |
| 34 | 189 |
| 35 | 7 |
| 40 | 90 |
| 41 | >2000 |
| 42 | 60 |
| 53 | 373 |
| 69 | >3000 |
| 70 | 329 |
| 71 | >2500 |
| 72 | 2666 |
| 73 | 143 |
| 74 | 395 |
| 77 | 101 |
| 78 | 215 |
| 79 | 320 |
| 80 | 241 |
| 81 | 117 |
| 82 | 53 |
| 83 | 232 |
| 84 | 107 |
| 85 | 43 |
| 86 | 28 |
| 87 | 39 |
| 88 | 52 |
| 89 | 23 |
| 90 | 197 |
| 91 | 342 |
| 92 | 252 |
| 93 | 54 |
| 94 | 450 |
| 95 | 130 |
| 96 | 1228 |
| 97 | 126 |
| 98 | 72 |
| 99 | 623 |
| 100 | 45 |
| 101 | 505 |
| 102 | 7 |
| 103 | <8 |
| 104 | 164 |
| 105 | 57 |
| 108 | 53 |
| 109 | 216 |
| 110 | 90 |
| 111 | 40 |
| 112 | <8 |
| 113 | 74 |
| 114 | ND |
| 117 | ND |
| 123 | 111 |
| 124 | >10000 |
| 135 | 4 |
| 136 | 16 |
| 137 | 6 |
| 138 | 9 |
| 139 | 5 |
| 140 | 1191 |
| 141 | 3 |
| 142 | 1 |
| 143 | 8 |
| 144 | 11 |
| 145 | 6 |
| 146 | 15 |
| 147 | 7 |
| 148 | 9 |
| 149 | 11 |
| 150 | 1 |
| 151 | 5 |
| 152 | 4 |
| 153 | 4 |
| 154 | 4 |

ND = no data available

| | Replicon 1b |
|---|---|
| Example | IC₅₀ (nM) |
| 155 | 1.1 |
| 156 | 6.9 |
| 157 | 22.7 |
| 158 | 4.8 |
| 159 | 1.8 |
| 160 | 7.0 |
| 161 | 3.7 |
| 162 | 8.2 |
| 163 | 8.5 |
| 164 | 16.5 |
| 165 | 4.7 |
| 166 | 4.4 |
| 167 | 10.4 |
| 168 | 3.9 |
| 169 | 6.4 |
| 170 | 5.2 |
| 171 | 6.8 |
| 172 | 27.0 |
| 173 | 5.6 |
| 174 | 10.4 |
| 175 | 13.8 |
| 176 | 3.9 |
| 177 | 7.4 |
| 178 | 7.0 |
| 179 | 7.2 |
| 180 | 5.0 |
| 181 | 5.7 |
| 182 | 2.3 |
| 183 | 1.5 |
| 185 | 2.2 |
| 186 | 2.0 |
| 187 | 2.6 |
| 188 | 5.1 |
| 189 | 1.9 |
| 190 | 2.2 |
| 191 | 4.1 |
| 192 | 1.9 |
| 193 | 11.6 |
| 194 | 4.2 |
| 198 | 1.4 |
| 199 | 2.9 |
| 200 | 1.6 |
| 201 | 1.7 |
| 202 | 1.0 |
| 203 | 2.4 |
| 204 | 4.5 |
| 205 | 11.9 |
| 206 | 2.1 |
| 207 | 2.4 |
| 208 | 1.8 |
| 209 | 5.5 |
| 210 | 1.9 |
| 211 | 3.3 |
| 212 | 2.6 |
| 213 | 2.5 |
| 214 | 6.3 |
| 215 | 2.1 |
| 216 | 8.2 |
| 217 | 2.1 |
| 218 | 1.8 |
| 219 | 3.0 |
| 220 | 1.5 |
| 221 | 1.7 |
| 222 | 3.5 |
| 223 | 10.0 |
| 224 | 2.3 |
| 225 | 3.6 |
| 226 | 4.0 |
| 227 | 2.2 |
| 228 | 3.2 |
| 229 | 3.7 |
| 230 | 8.3 |
| 231 | 6.6 |
| 232 | 2.4 |
| 233 | 2.9 |
| 234 | 2.7 |
| 235 | 4.0 |
| 237 | 2.6 |
| 238 | 2.9 |
| 239 | 2.6 |
| 240 | 1.9 |
| 241 | 9.5 |
| 242 | 4.8 |
| 243 | 1.3 |
| 244 | 1.3 |
| 245 | 2.0 |
| 246 | 3.6 |
| 249 | 2.2 |
| 250 | 5.1 |
| 251 | 4.0 |
| 252 | 78.2 |
| 260 | 3.7 |
| 261 | 2.0 |
| 262 | 5.0 |
| 264 | 2.9 |
| 271 | 1.5 |
| 272 | 3.0 |
| 273 | 2.2 |
| 278 | 3.2 |
| 279 | 3.2 |
| 281 | 3.3 |
| 282 | 2.3 |
| 283 | 17.2 |
| 284 | 5.3 |
| 285 | 10.2 |
| 286 | 5.8 |
| 289 | 5.4 |
| 290 | 6.9 |
| 292 | 5.9 |
| 293 | 8.6 |
| 294 | 10.0 |
| 295 | 4.1 |
| 296 | 64.1 |
| 297 | 20.5 |
| 298 | 6.0 |
| 299 | 3.3 |
| 300 | 1.5 |
| 301 | 1.9 |
| 302 | 3.1 |
| 303 | 1.8 |
| 304 | 1.3 |
| 305 | 1.1 |
| 306 | 28.8 |
| 307 | 2.4 |
| 308 | 2.5 |
| 309 | 3.7 |
| 310 | 1.8 |
| 311 | 1.7 |
| 312 | 1.1 |
| 314 | 1.5 |
| 315 | 4.5 |
| 316 | 2.8 |
| 317 | 2.9 |
| 318 | 18.7 |
| 319 | 13.6 |
| 320 | 13.1 |
| 321 | 6.6 |
| 322 | 19.5 |
| 323 | 2.4 |
| 324 | 3.5 |
| 325 | 3.2 |
| 327 | 3.8 |
| 328 | 13.5 |
| 329 | 4.9 |
| 330 | 2.7 |
| 331 | 1.8 |
| 332 | 9.6 |
| 333 | 6.0 |
| 334 | 3.3 |
| 335 | 26.4 |
| 336 | 2.7 |
| 337 | 7.8 |
| 338 | 1.5 |
| 339 | 4.0 |
| 340 | 15.8 |
| 341 | 14.6 |
| 342 | 5.1 |
| 343 | 3.2 |
| 344 | 2.2 |
| 345 | 2.1 |
| 346 | 165.2 |
| 347 | 8.1 |
| 348 | 72.7 |
| 349 | 5.7 |
| 350 | 0.7 |
| 353 | 4.2 |
| 356 | 5.9 |
| 360 | 30.5 |
| 366 | 4.2 |
| 367 | 3.9 |
| 368 | 1.6 |
| 369 | 2.7 |
| 370 | 8.8 |
| 371 | 5.8 |
| 372 | 13.0 |
| 373 | 3.9 |
| 374 | 52.4 |
| 375 | 2.7 |
| 377 | 1.2 |
| 378 | 1.8 |
| 380 | 1.2 |
| 381 | 5.1 |
| 382 | 8.9 |
| 383 | 2.5 |
| 384 | 0.9 |
| 386 | 1.3 |
| 387 | 2.3 |
| 391 | 2.4 |
| 393 | 1.2 |
| 394 | 1.4 |
| 395 | 8.2 |
| 396 | 18.2 |
| 397 | 3.6 |
| 398 | 2.4 |
| 399 | 2.8 |
| 400 | 303.4 |
| 401 | 1.8 |
| 402 | 0.9 |
| 403 | 3.8 |
| 404 | 2.6 |
| 405 | 4.7 |
| 406 | 15.1 |
| 407 | 2.8 |
| 408 | 3.8 |
| 409 | 2.1 |
| 410 | 0.5 |

| | 1b |
|---|---|
| Example | IC₅₀(nM) |
| 411 | 7.096 |
| 420 | 5.078 |
| 425 | 3.041 |
| 427 | 4.35 |
| 428 | 29.07 |
| 429 | 4.9063 |
| 436 | 4.368 |
| 478 | 8.651 |
| 479 | 1.949 |
| 480 | 3.009 |
| 483 | 2.475 |
| 484 | 3.315 |
| 504 | 1.526 |
| 507 | 0.6799 |
| 509 | 3.0 |
| 515 | 3.989 |
| 517 | 6.999 |
| 547 | 23.16 |
| 549 | 65.93 |
| 551 | 1.3 |
| 558 | 5.812 |
| 581 | 0.5 |
| 588 | 50.43 |
| 589 | 3.378 |
| 590 | 44.06 |
| 598 | 3.427 |
| 606 | 9.318 |
| 607 | 27.07 |
| 611 | 11.11 |
| 620 | 1.656 |
| 622 | 647.2 |
| 627 | 1.395 |
| 632 | 4.613 |
| 637 | 4.03 |
| 638 | 29.78 |
| 650 | 55.67 |
| 651 | 154.9 |
| 654 | 3.652 |
| 655 | 2.519 |
| 656 | 8.413 |
| 658 | 8.531 |
| 659 | 8.622 |
| 663 | 2.503 |
| 664 | 2.984 |
| 685 | 11.99 |
| 686 | 17.59 |
| 687 | 27.33 |
| 688 | 7.531 |
| 701 | 6.212 |
| 705 | 15.56 |
| 706 | 16.74 |
| 707 | 4.927 |
| 708 | 3.946 |
| 709 | 6.051 |
| 717 | 2.843 |
| 718 | 3.901 |
| 719 | 3.138 |
| 720 | 41.64 |
| 721 | 4.944 |
| 722 | 3.03 |
| 723 | 2.309 |
| 724 | 4.466 |
| 725 | 10.51 |
| 726 | 5.538 |
| 729 | 3.302 |
| 733 | 7.78 |
| 735 | 10.5 |
| 737 | 19.02 |
| 738 | 7.625 |
| 739 | 2.165 |
| 740 | 4.76 |
| 741 | 5.849 |
| 742 | 4.568 |
| 769 | 80.74 |
| 770 | 11.3 |
| 771 | 13.32 |
| 778 | 24.8 |
| 779 | 8.278 |
| 780 | 18.52 |
| 782 | 5.413 |
| 784 | 3.607 |
| 785 | 3.548 |
| 788 | 78.97 |
| 789 | 18.71 |
| 791 | 28.49 |
| 796 | 13.96 |
| 797 | 2.432 |
| 807 | 46.7 |
| 809 | 212.7 |
| 810 | 13.41 |
| 813 | 107.1 |
| 814 | 9.826 |
| 815 | 11.75 |
| 819 | 8.296 |
| 820 | 5.058 |
| 821 | 3.266 |
| 824 | 17.69 |
| 825 | 8.877 |
| 827 | 11.84 |
| 830 | 8.475 |
| 834 | 11.5 |
| 836 | 82.01 |
| 843 | 19.53 |
| 844 | 3.885 |
| 848 | 6443 |
| 850 | 3135 |
| 851 | 349.9 |
| 852 | 831.1 |
| 855 | 1.2 |
| 856 | 4.0 |
| 857 | 2.6 |
| 858 | 2.8 |
| 859 | 2.1 |
| 860 | 1.9 |
| 861 | 1.8 |
| 862 | 1.3 |
| 863 | 1.8 |
| 864 | 9.4 |
| 865 | 4.7 |
| 866 | 3.1 |
| 867 | 1.8 |
| 868 | 1.3 |
| 869 | 2.3 |
| 870 | 1.3 |
| 871 | 3.5 |
| 872 | 4.7 |
| 873 | 3.4 |
| 874 | 2.1 |
| 875 | 2.1 |
| 876 | 3.5 |
| 877 | 5.1 |
| 878 | 17.2 |
| 879 | 2.6 |
| 880 | 2.9 |

It will be appreciated that various of the above-discussed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A compound having structural formula (I): 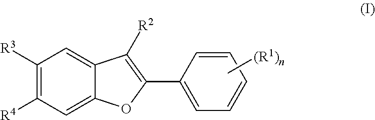
or a pharmaceutically acceptable salt thereof, wherein:
each R¹ is independently selected from the group consisting of halo, C₁-C₆ alkyl, —O—(C₁-C₆ alkyl), —O—(C₁-C₆ haloalkyl) and —CN;
n is 0, 1, 2, 3 or 4;
R² is C(O)NR^{a}R^{b};
R^{a} and R^{b} are independently selected from the group consisting of hydrogen, C₁-C₆ alkyl, O(C₁-C₆ alkyl) and 5- or 6-membered monocyclic aromatic rings with 0, 1, 2, 3 or 4 heteroatom ring atoms independently selected from the group consisting of N, O or S;
R³ is ArA, —C≡C-phenyl or a 15- or 16-membered tetracyclic ring system, wherein said 15- or 16-membered tetracyclic ring system is substituted by 0, 1 or 2 substituents independently selected from C₁-C₆ alkyl, phenyl, C₃-C₇ cycloalkyl or 6-membered heteroaryl, and
wherein ArA is an aromatic ring system selected from the group consisting of:
i) 5- or 6-membered monocyclic rings with 0, 1, 2, 3 or 4 heteroatom ring atoms independently selected from the group consisting of N, O or S; and
ii) 8-, 9- or 10-membered bicyclic rings with 0, 1, 2, 3 or 4 heteroatom ring atoms independently selected from the group consisting of N, O or S, and
wherein said ArA is substituted by 0, 1, 2, 3 or 4 substitutents R^{c};
each R^{c} is independently selected from the group consisting of:
a) halogen,
b) OH
c) C₁-C₆ alkyl,
d) O(C₁-C₆ alkyl),
e) CN,
f) (CH₂)₀₋₃-ArB, wherein each ArB is an independently selected aromatic ring system selected from the group consisting of:
i) 5- or 6-membered monocyclic rings with 0, 1, 2, 3 or 4 heteroatom ring atoms independently selected from the group consisting of N, O or S, and
ii) 8-, 9- or 10-membered bicyclic rings with 0, 1, 2, 3 or 4 heteroatom ring atoms independently selected from the group consisting of N, O or S,
g) (CH₂)₀₋₃NR^{d}C(O)R^{e},
h) (CH₂)₀₋₃NR^{d}SO₂R^{e},
i) (CH₂)₀₋₃C(O)NR^{d}R^{e},
j) (CH₂)₀₋₃SO₂R^{e},
k) —OSO₂(C₁-C₆ alkyl), and
l) C₂-C₆ alkynyl
wherein each R^{e} c) C₁-C₆ alkyl, d) O (C₁-C₆ alkyl), and f) (CH₂)₀₋₃-ArB is substituted by 0, 1, 2, 3 or 4 substituents R^{f};
wherein any 2 R^{e} groups on adjacent ring carbon atoms can join to form a group selected from —OC(O)—N—, —OCH₂CH₂O—, —OCH₂—O— and —OCH₂CH₂,
each R^{d} is independently selected from the group consisting of hydrogen and C₁₋₆alkyl;
each R^{e} is independently selected from the group consisting of hydrogen, C₁₋₆alkyl, OC₁₋₆alkyl and 5- or 6-membered monocyclic rings with 0, 1, 2, 3 or 4 heteroatom ring atoms independently selected from the group consisting of N, O or S, wherein each R^{e} C₁₋₆alkyl, OC₁₋₆alkyl and 5- or 6-membered monocyclic rings is substituted by 0, 1, 2, 3 or 4 substituents independently selected from the group consisting of C₁-C₆ alkyl, O(C₁-C₆ alkyl), halogen and OH;
each R^{f} is independently selected from the group consisting of:
a) halogen,
b) C₁-C₆ alkyl,
c) O(C₁-C₆ alkyl),
d) CN,
e) N(R^{q})₂,
f) OH,
g) C(O)H,
h) NHC(O)R^{s},
i) NHS(O)₂R^{s},
j) C(O)NHR^{q},
k) C(O)OR^{q},
l) OS(O)₂(C₁-C₆ alkyl),
m) (CH₂)₀₋₃-ArC, wherein each ArC is an independently selected aromatic ring system selected from the group consisting of:
i) 5- or 6-membered monocyclic rings with 0, 1, 2, 3 or 4 heteroatom ring atoms independently selected from the group consisting of N, O or S, and
ii) 8-, 9- or 10-membered bicyclic rings with 0, 1, 2, 3 or 4 heteroatom ring atoms independently selected from the group consisting of N, O or S,
wherein each R^{f}: b) C₁-C₆ alkyl, c) O(C₁-C₆ alkyl), and m) (CH₂)₀₋₃-ArC is substituted by 0, 1, 2, 3 or 4 substituents R⁹;
each R⁹ is independently selected from the group consisting of halogen, OH, N(R^{q})₂, CN, C₁₋₆alkyl, O(C₁-C₆ alkyl), CF₃ and C(O)OH;
R⁴ is selected from the group consisting of NR^{h}Rⁱ and 5- or 6-membered monocyclic rings with 0, 1, 2, 3 or 4 heteroatom ring atoms independently selected from the group consisting of N, O or S, and
R^{h} is selected from the group consisting of:
a) hydrogen,
b) C₁₋₆alkyl,
c) C(O)O(C₁₋₆alkyl), and
d) SO₂R^{j};
R^{j} is selected from the group consisting of C₁₋₆alkyl, C₆₋₁₀ aryl, C₃₋₇ cycloalkyl and NR^{x}R^{y}, where R^{x} and R^{y} are independently selected from the group consisting of hydrogen and C₁₋₆alkyl;
Rⁱ is selected from the group consisting of:
a) C₁₋₆alkyl,
b) C₂₋₆alkenyl,
c) C₂₋₆alkynyl,
d) (CH₂)₀₋₃(C₃₋₈cycloalkyl),
e) (CH₂)₀₋₃(C₃₋₈cycloalkenyl),
f) C(O)C₁₋₆alkyl, and
g) heterocyclyl,
wherein Rⁱ is substituted by 0, 1, 2, 3 or 4 R^{k} groups;
each R^{k} is independently selected from the group consisting of:
a) OR^{L},
b) halogen,
c) CN,
d) NR^{m}Rⁿ,
e) OC(O)C₁₋₆alkyl,
f) C(O)OC₁₋₆alkyl,
g) —P(O)(O—C₁₋₆ alkyl)₂,
h) —P(O)(OH)(O—C₁₋₆ alkyl),
j) —P(O)(OH)₂,
k) —C(O)C(C₁₋₆ alkyl)-NHC(O)—C₁₋₆ alkyl,
l) —NHC(O)C(C₁₋₆ alkyl)-NHC(O)—C₁₋₆ alkyl,
m) —C(O)OH,
n) (CH₂)₀₋₃-ArD, wherein each ArD is an independently selected aromatic ring system selected from the group consisting of:
i) 5- or 6-membered monocyclic rings with 0, 1, 2, 3 or 4 heteroatom ring atoms independently selected from the group consisting of N, O or S, and ii) 8-, 9- or 10-membered bicyclic rings with 0, 1, 2, 3 or 4 heteroatom ring atoms independently selected from the group consisting of N, O or S,
wherein each R^{k} e) OC(O)C₁₋₆alkyl, f) C(O)OC₁₋₆alkyl, and g) (CH₂)₀₋₃-ArD is substituted by 0, 1, 2, 3 or 4 R^{o} groups;
R^{L} is selected from the group consisting of hydrogen, C₁₋₆alkyl and phenyl;
R^{m} is selected from the group consisting of hydrogen, C₁₋₆alkyl and (CH₂)₀₋₃(phenyl);
Rⁿ is selected from the group consisting of hydrogen, C₁₋₆alkyl, SO₂(C₁₋₆alkyl), —C(O)H, —C(O)OH, —C(O)O(C₁₋₆alkyl) and C(O)(C₁₋₆alkyl);
or R^{m} and Rⁿ are taken together with the N to which they are attached to form a 5- to 7-membered ring substituted by 0, 1, 2 or 3 R^{p};
each R^{o} is independently selected from the group consisting of halogen, C₁₋₆alkyl, OC₁₋₆alkyl and C(O)O(C₁₋₆alkyl);
each R^{p} is independently selected from the group consisting of halogen, C₁₋₆alkyl, OC₁₋₆alkyl, oxo and C(O)O(C₁₋₆alkyl);
each R^{q} is independently selected from the group consisting of H and C₁₋₆alkyl;
each R^{s} is independently selected from the group consisting of C₁₋₆alkyl, heterocyclyl and C₆₋₁₀aryl, wherein said heterocyclyl group can be optionally substituted on a ring nitrogen or ring carbon atom with a —C(O)O—(C₁-C₆ alkyl) group; and
each R^{t} is independently selected from the group consisting of C₁₋₆alkyl and C₆₋₁₀aryl;
or R^{h} and Rⁱ are taken together with the N to which they are attached to form a 5- to 7-membered ring.

2. (canceled)

3. The compound according to claim 1, wherein the compound is a compound of formula (Ib): 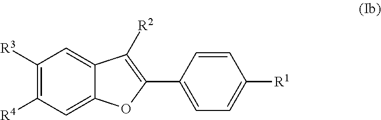
, wherein R¹ is selected from the group consisting of fluorine, bromine and chlorine.

4.-5. (canceled)

6. The compound according to claim 1, wherein R^{a} is hydrogen and R^{b} is selected from the group consisting of —CH₃ and —OCH₃.

7. (canceled)

8. The compound according to claim 1, wherein said ArA is phenyl, which is substituted by 0, 1, 2, 3 or 4 substitutents R^{e}, and wherein each said R^{e} is independently selected from the group consisting of:
a) fluorine,
b) OH,
c) C₁₋₃alkyl,
d) OC₁₋₃alkyl,
e) CN,
f) (CH₂)₀₋₁-ArB, wherein ArB is independently selected from the group consisting of: 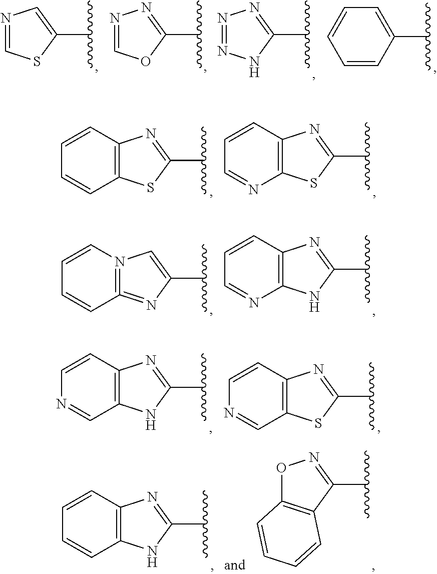
wherein said ArB is substituted by 0, 1, 2, 3 or 4 substituents R^{f},
g) (CH₂)₀₋₁N(CH₃)SO₂CH₃
h) (CH₂)₀₋₁N(H)SO₂CH₃,
i) (CH₂)₀₋₁N(CH₃)SO₂-phenyl,
j) C(O)NHCH₃,
k) (CH₂)₀₋₁N(H)C(O)CH₃, and
l) (CH₂)₀₋₁N(H)C(O)phenyl.

9. (canceled)

10. The compound according to claim 1, wherein each said R^{e} is independently selected from the group consisting of 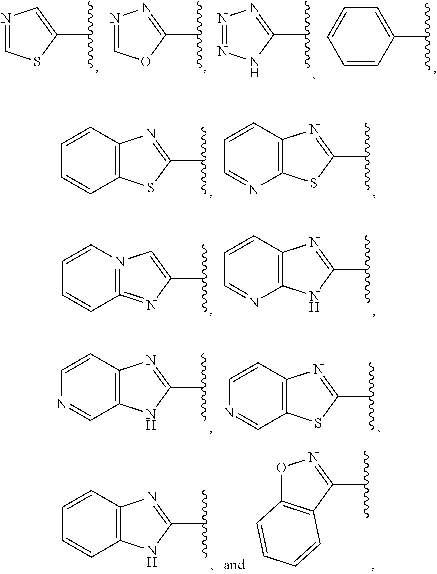
wherein each said R^{e} group is substituted by 0, 1, 2, 3 or 4 substituents R^{f}.

11. The compound according to claim 1, wherein R^{h} is selected from hydrogen, CH₃ and SO₂CH₃.

12. (canceled)

13. The compound according to claim 1, wherein Rⁱ is selected from the group consisting of C₁₋₆alkyl and C₂₋₆alkenyl, and R^{k} is selected from the group consisting of
a) OR¹,
b) halogen,
c) CN,
d) NR^{m}Rⁿ,
e) OC(O)C₁₋₆alkyl, and
f) C(O)OC₁₋₆alkyl.

14. (canceled)

15. The compound according to claim 1, wherein R^{L} is selected from the group consisting of C₁₋₆alkyl, R^{m} is selected from the group consisting of hydrogen and C₁₋₆alkyl and Rⁿ is selected from the group consisting of C₁₋₆alkyl and SO₂(C₁₋₆alkyl).

16.-17. (canceled)

18. A compound having the formula: 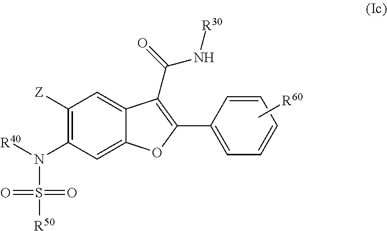
or a pharmaceutically acceptable salt thereof,
wherein:
Z is a phenyl group which is substituted with one R¹⁰ group and optionally further substituted with R²⁰;
R¹⁰ is an 8- to 10-membered bicyclic heteroaryl group, wherein said 8- to 10-membered bicyclic heteroaryl group is optionally substituted with up to 4 groups, which can be the same or different, and are selected from halo, C₁-C₆ alkyl, —C(O)H, —(CH₂)ₜ—N(R⁷⁰)₂, —(CH₂)ₜ—OH, —(CH₂)ₜ—O—(C₁-C₆ alkyl), —CF₃, —NHC(O)-heterocyclyl, —NHC(O)—(C₁-C₆ alkyl), —C(O)NH—(C₁-C₆ alkyl), —C(O)OH, —C(O)O—(C₁-C₆ alkyl), —NHC(O)-aryl, —NHSO₂-aryl, —NHSO₂-alkyl, —O—SO₂-alkyl, —O—(C₁-C₆ alkyl) and —CN, wherein the heterocyclyl moiety of said —NHC(O)-heterocyclyl group can be optionally substituted on a ring carbon or ring nitrogen atom with a —C(O)O—(C₁-C₆ alkyl) group;
R²⁰ represents up to 4 optional substituents, which can be the same or different, and are selected from halo, 8- to 10-membered heteroaryl, C₁-C₆ alkyl, —O—(C₁-C₆ alkyl), —O—(CH₂)ₜ—OH, —O—(CH₂)ₜ-heterocyclyl, —O—(C₁-C₆ haloalkyl), —O—SO₂—(C₁-C₆ alkyl) and —CN;
R³⁰ is H or C₁-C₆ alkyl;
R⁴⁰ is selected from C₁-C₆ alkyl, C₁-C₆ haloalkyl, —(CH₂)ₙ—OH, —(CH₂)ₜ-heterocyclyl, —(CH₂)ᵤ—N(R⁷⁰)₂, —(CH₂)ᵤ—CN, —(CH₂)ᵤ—NHC(O)OR³⁰ and —(CH₂)ₙ—NHC(O)R³⁰;
R⁵⁰ is C₁-C₆ alkyl, C₆-C₁₀ aryl or C₃-C₇ cycloalkyl;
R⁶⁰ represents up to 4 optional ring substituents, which can be the same or different, and are selected from halo, C₁-C₆ alkyl, —O—(C₁-C₆ alkyl), —O—(C₁-C₆ haloalkyl) and —CN;
each occurrence of R⁷⁰ is independently H or C₁-C₆ alkyl;
each occurrence of t is independently an integer ranging from 0 to 6; and
each occurrence of u is independently an integer ranging from 1 to 6.

19. The compound of claim 18, wherein Z is: 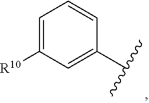
which can be optionally substituted on the depicted phenyl ring with one or two R²⁰ groups, which can be the same or different.

20. The compound according to claim 19, wherein R¹⁰ is selected from: 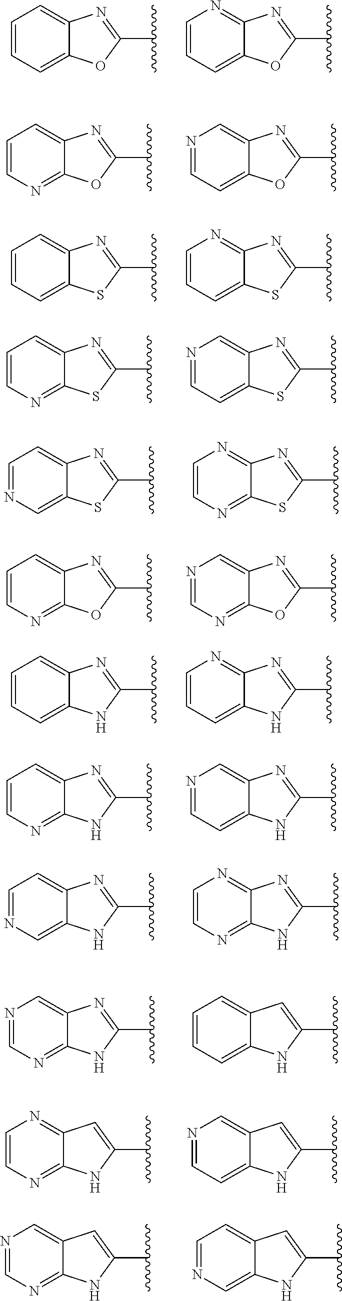 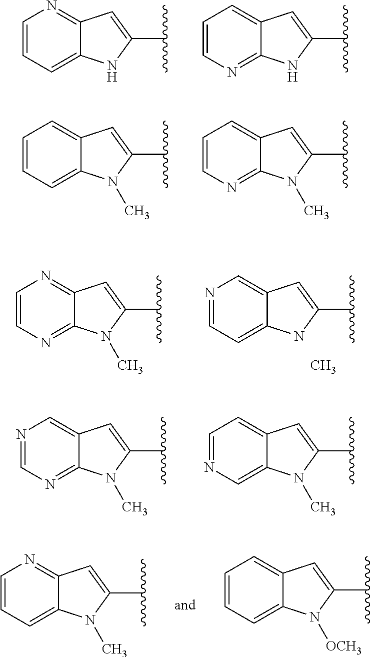
wherein R¹⁰ can be optionally substituted as set forth in claim 18.

21.-24. (canceled)

25. The compound of claim 18 having the formula: 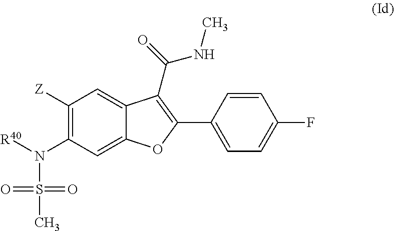
or a pharmaceutically acceptable salt thereof,
wherein:
Z is: 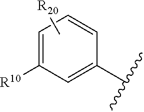
R¹⁰ is a 9-membered bicyclic heteroaryl group, wherein said 9-membered bicyclic heteroaryl group is optionally substituted with up to 2 groups, which can be the same or different, and are selected from halo, C₁-C₆ alkyl, —(CH₂)ₜ—N(R⁷⁰)₂, —(CH₂)ₜ—OH, —(CH₂)ₜ—O—(C₁-C₆ alkyl), —CF₃, —NHC(O)-heterocyclyl, —NHC(O)—(C₁-C₆ alkyl), —C(O)NH—(C₁-C₆ alkyl), —C(O)OH, —C(O)O—(C₁-C₆ alkyl), —NHC(O)-aryl, —NHSO₂-aryl, —NHSO₂-alkyl, —O—SO₂-alkyl, —O—(C₁-C₆ alkyl) and —CN, wherein the heterocyclyl moiety of said —NHC(O)-heterocyclyl group can be optionally substituted on a ring carbon or ring nitrogen atom with a —C(O)O—(C₁-C₆ alkyl) group
R²⁰ represents up to 2 optional substituents, which can be the same or different, and are selected from halo, C₁-C₆ alkyl, —O—(C₁-C₆ alkyl), —O—(CH₂)ₜ—OH, —O—(CH₂)ₜ-heterocyclyl, —O—(C₁-C₆ haloalkyl), —O—SO₂—(C₁-C₆ alkyl) and —CN;
R⁴⁰ is C₁-C₆ alkyl, C₁-C₆ haloalkyl, —(CH₂)ₜ—OH or —(CH₂)ₜ—CN; and
each occurrence oft is independently an integer ranging from 0 to 6.

26. The compound according to claim 25, wherein Z is selected from: 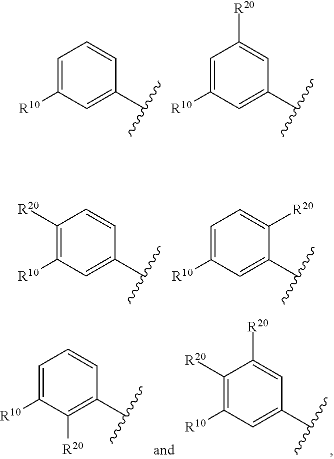
wherein each occurrence of R²⁰ is independently Cl, F, CN, —OCF₃ or —OCH₃.

27. The compound according to claim 26, wherein Z is selected from:

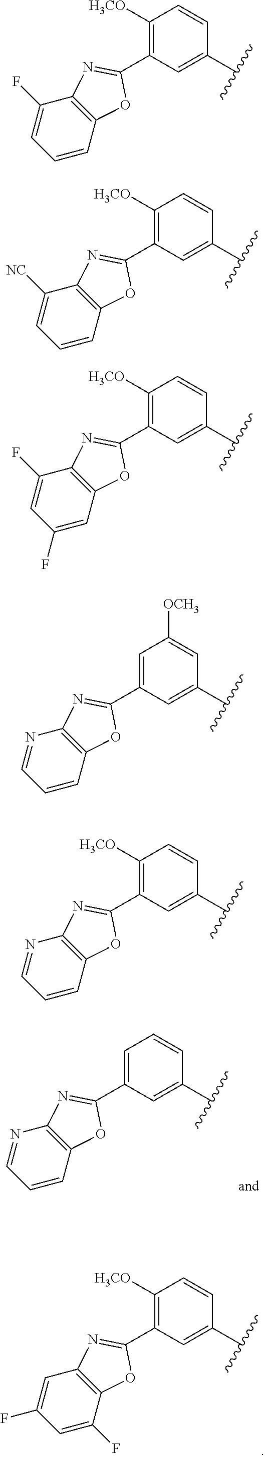

28. The compound according to claim 27, wherein R⁴⁰ is —CH₃, —(CH₂)₃—CN, —CH₂CH₂F or —CH₂CH₂C(CH₃)₂—OH.

29. A compound selected from the group consisting of 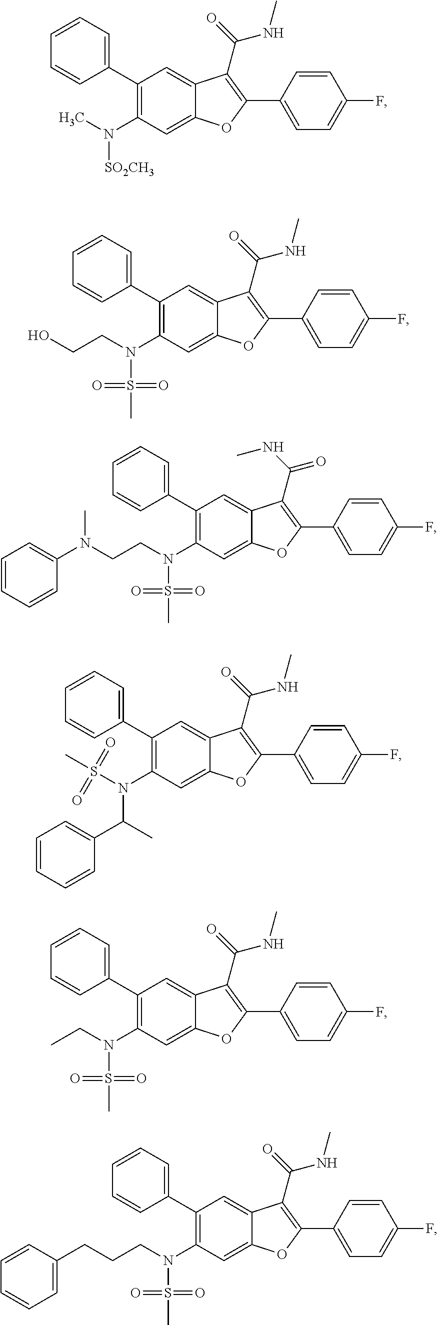 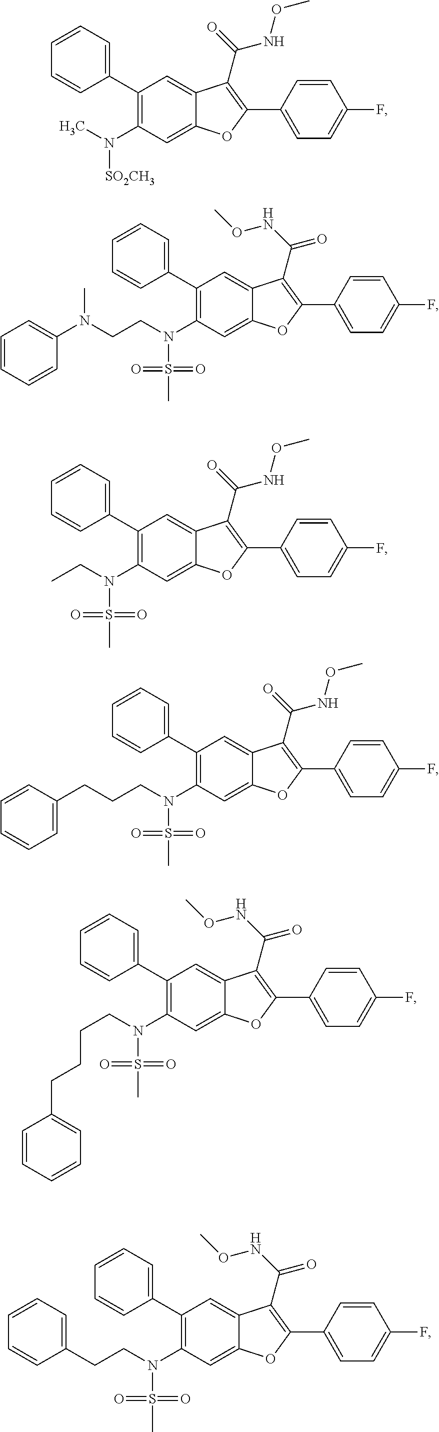 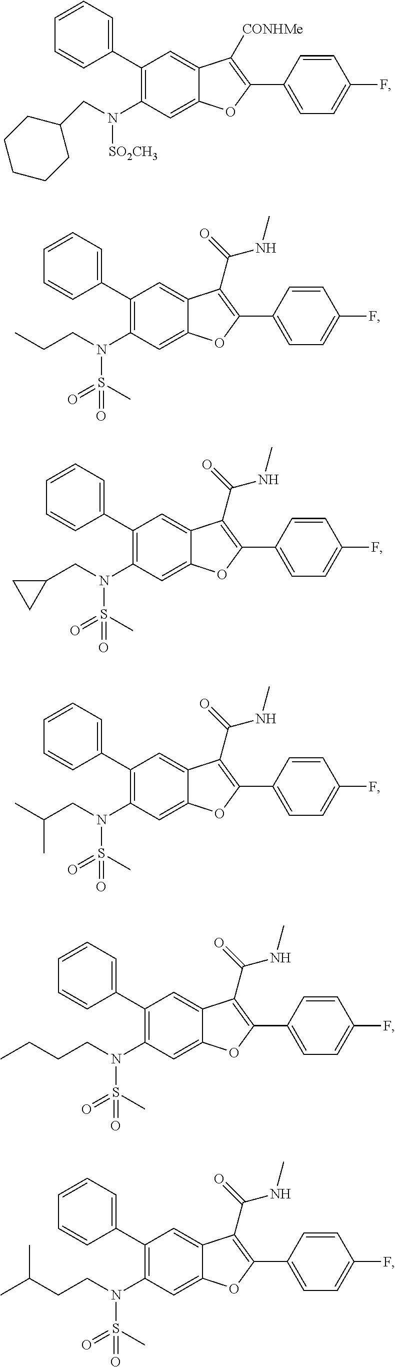 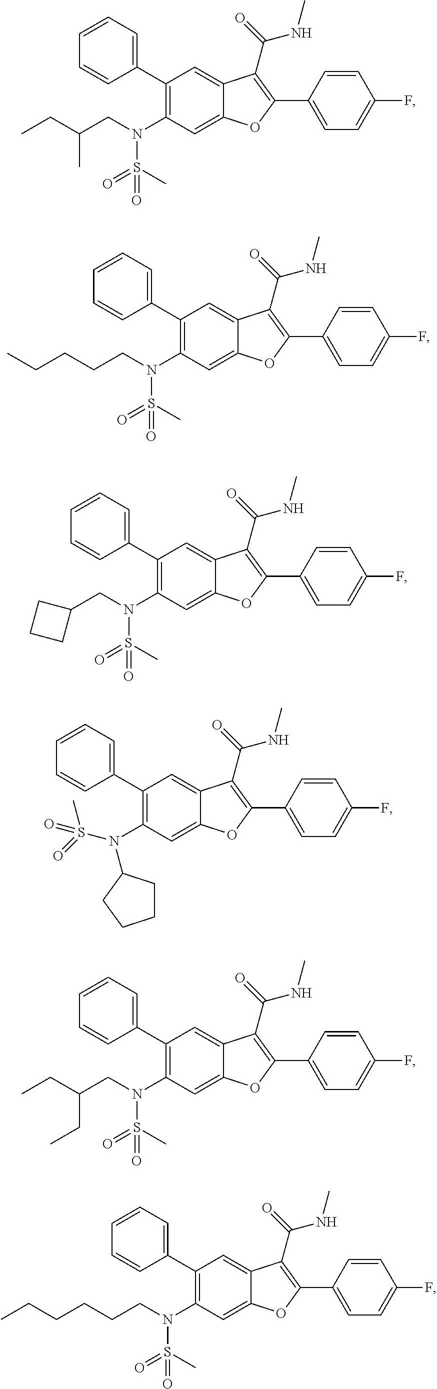 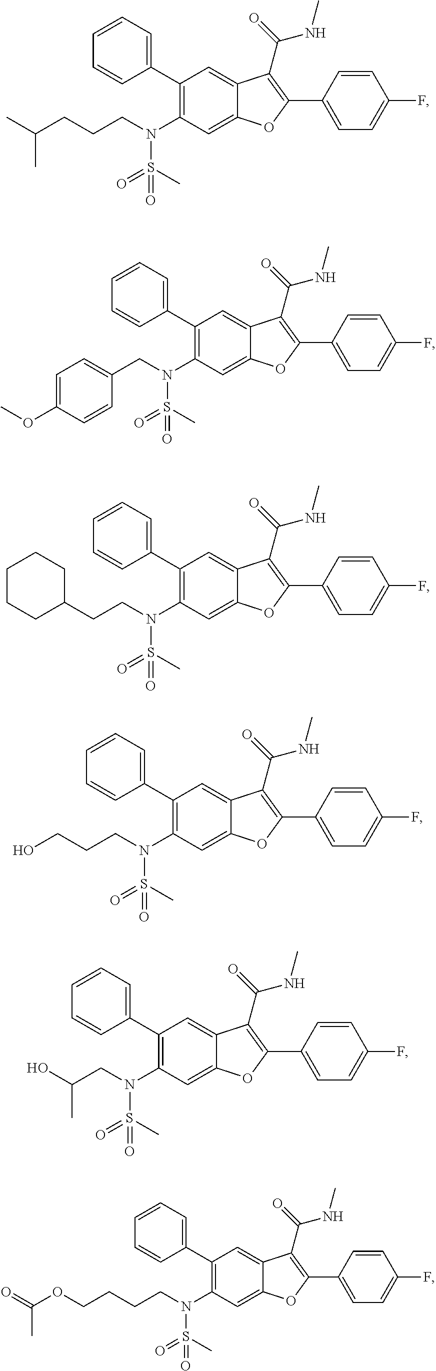 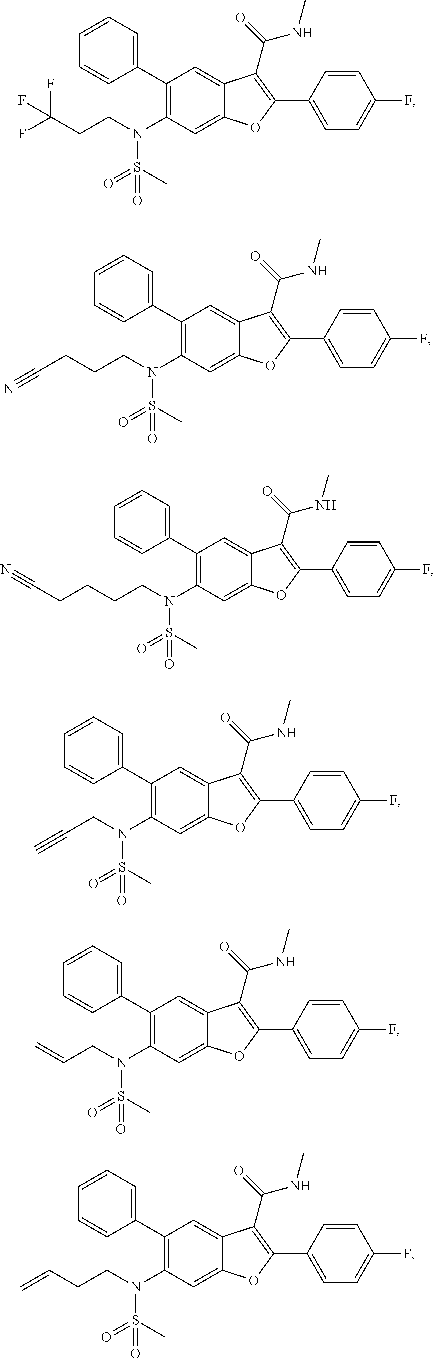 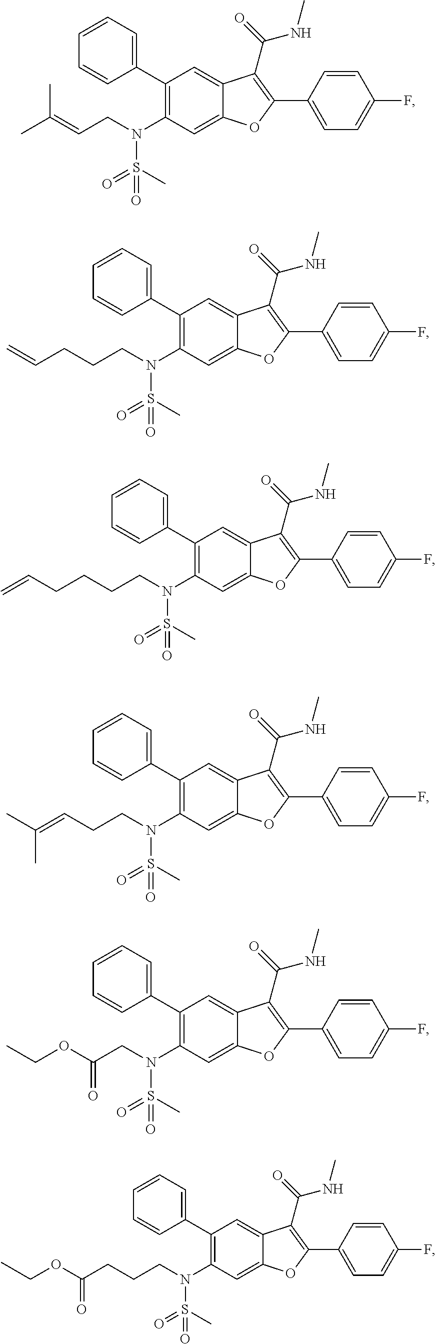 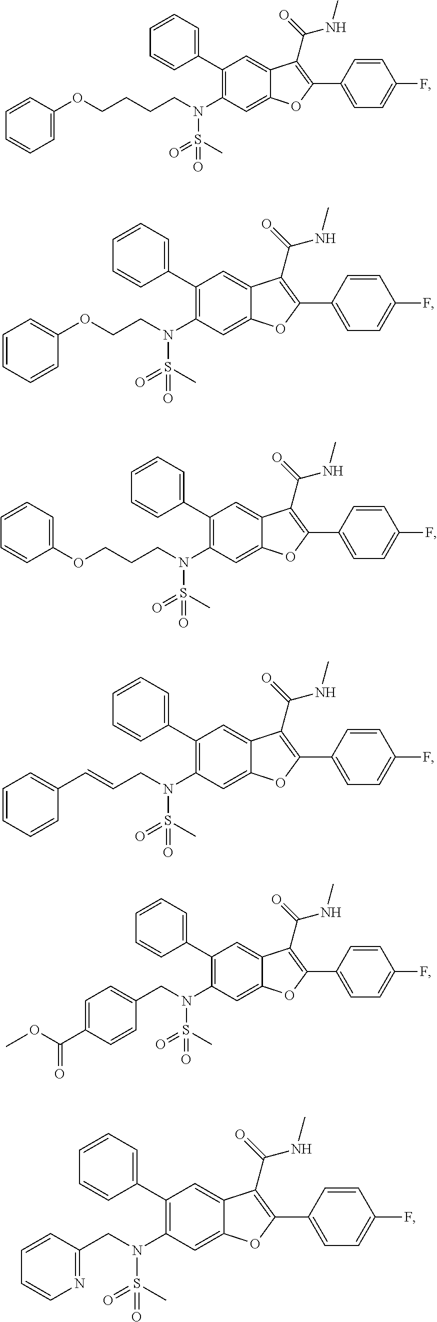 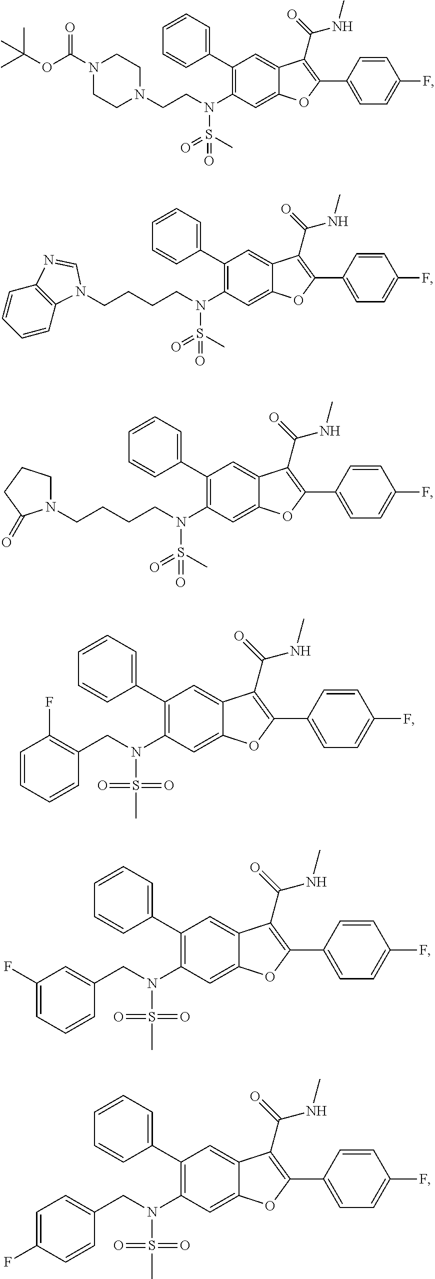 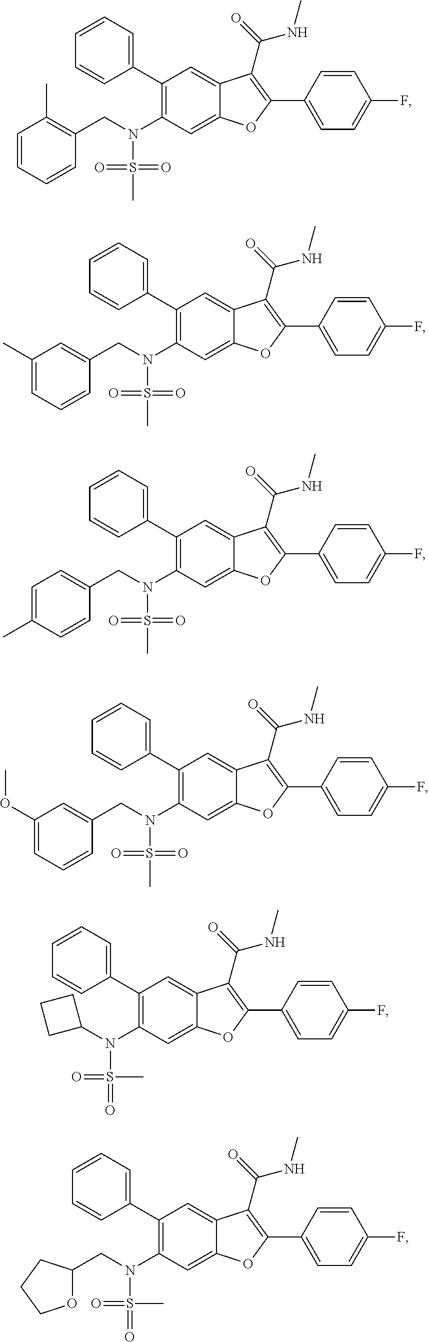 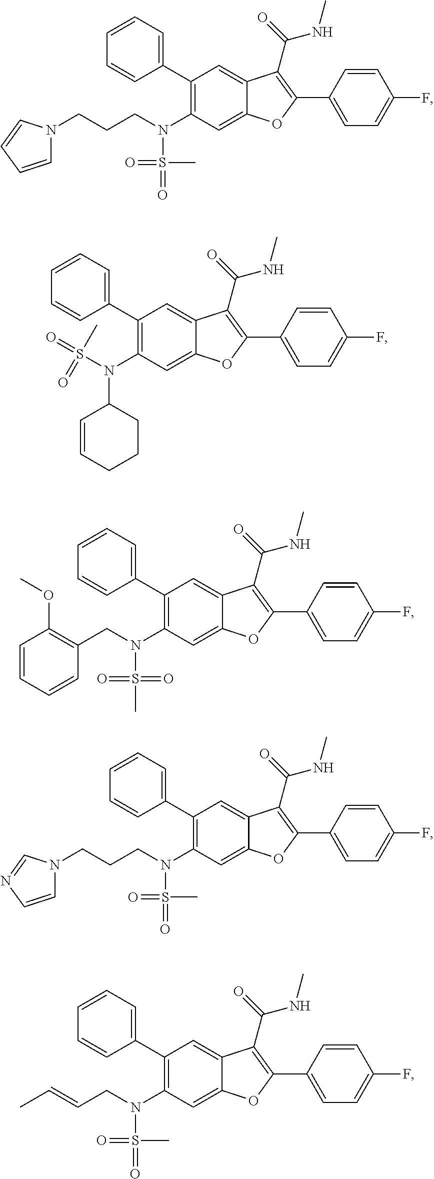 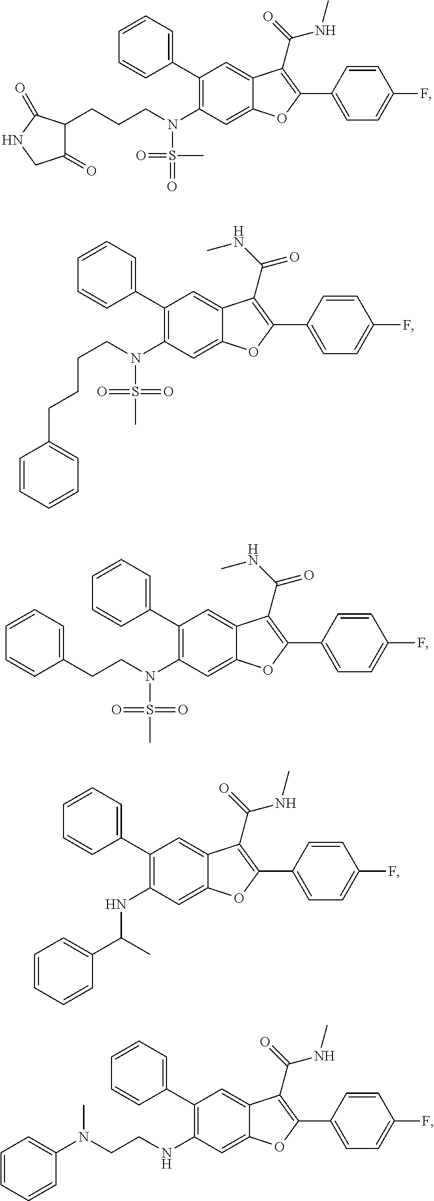 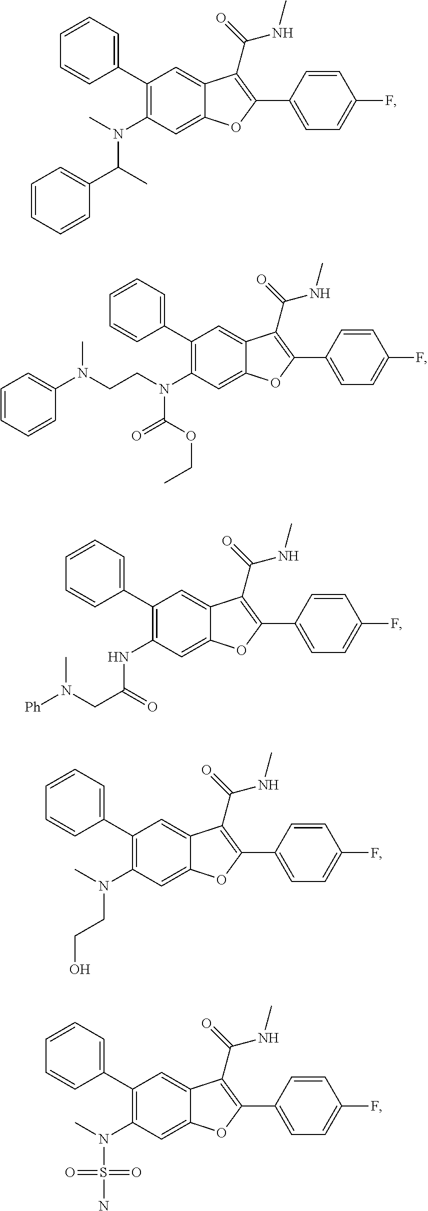 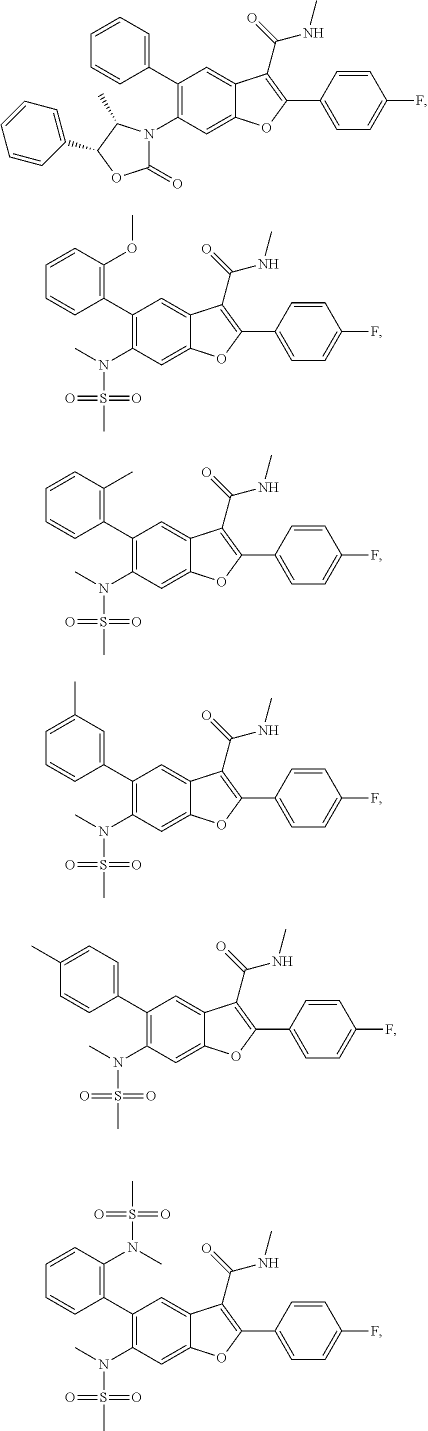 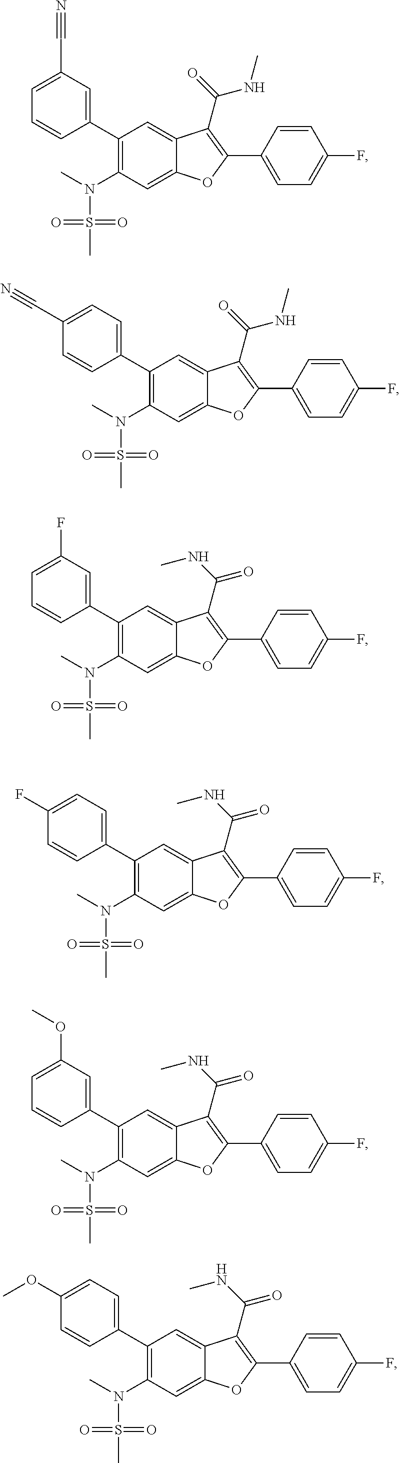 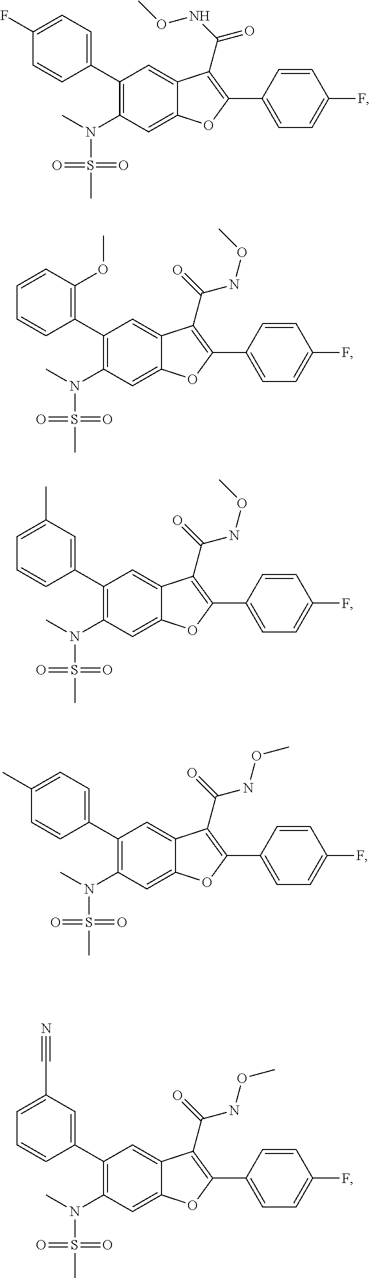 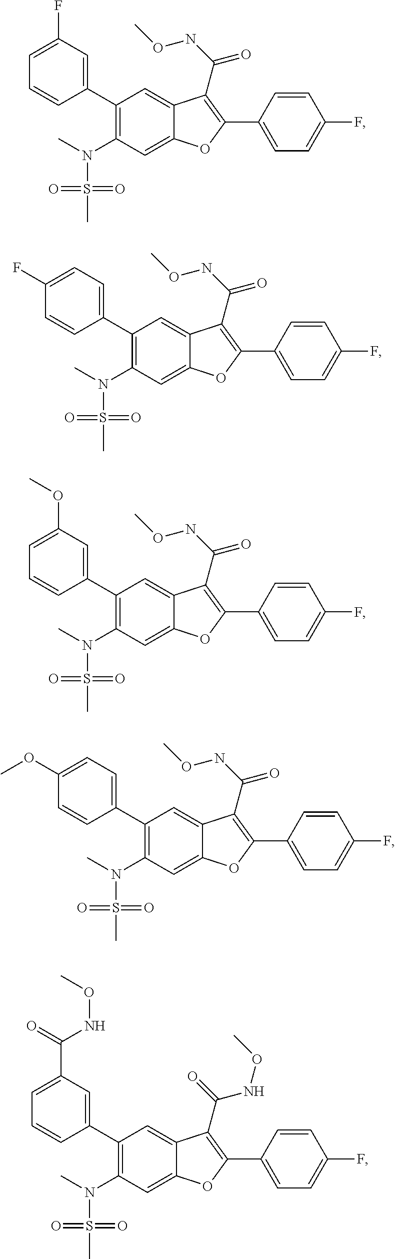 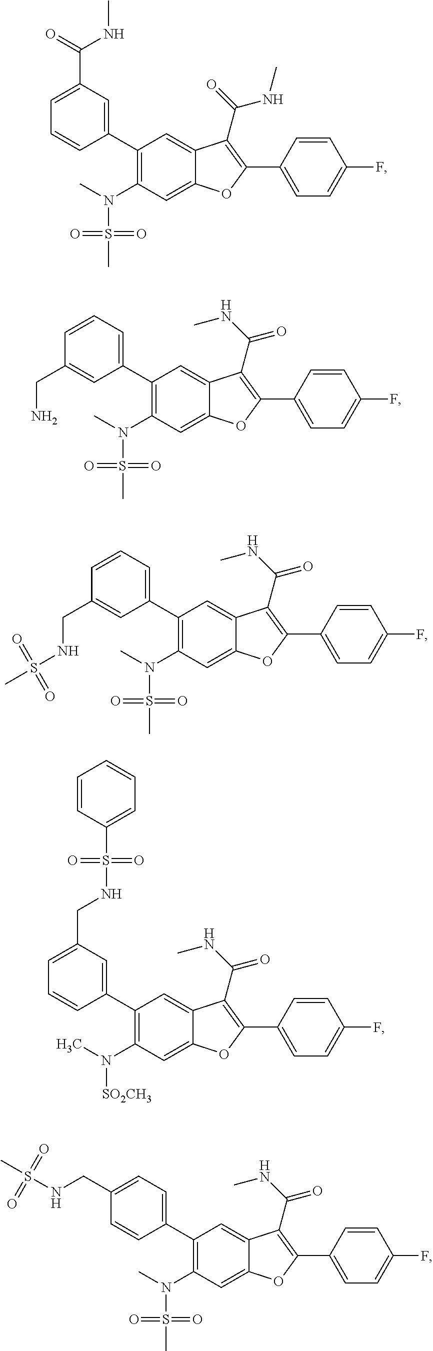 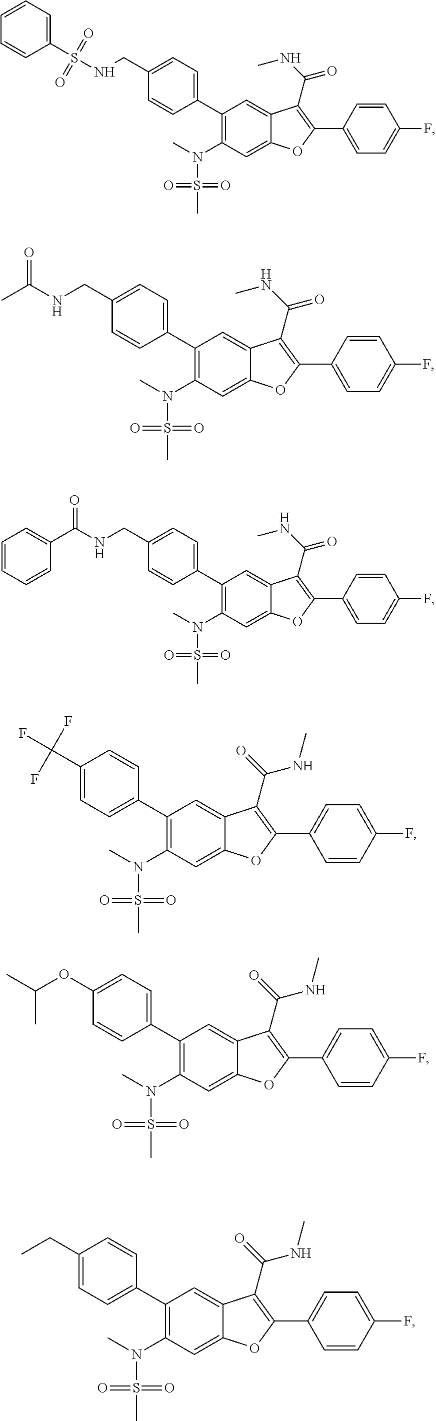 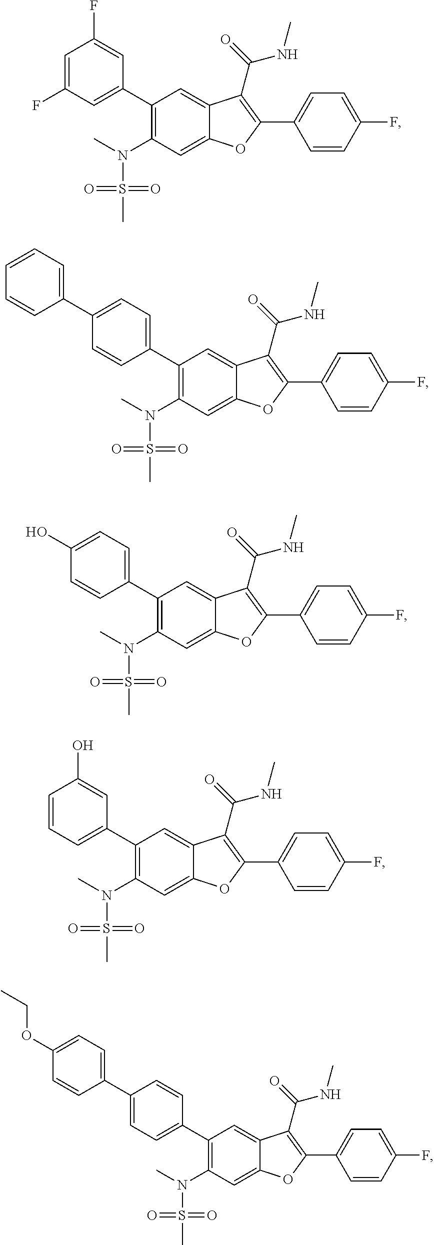 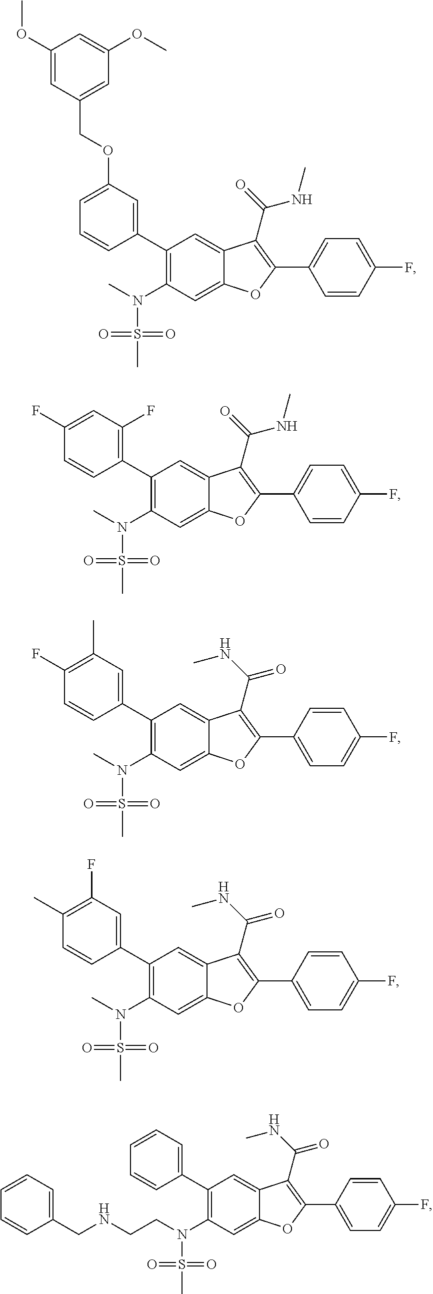 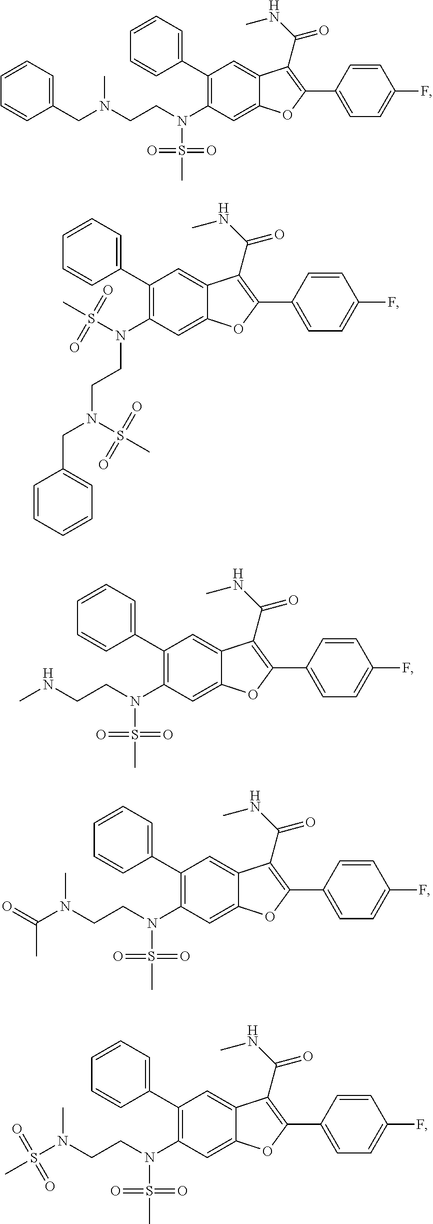 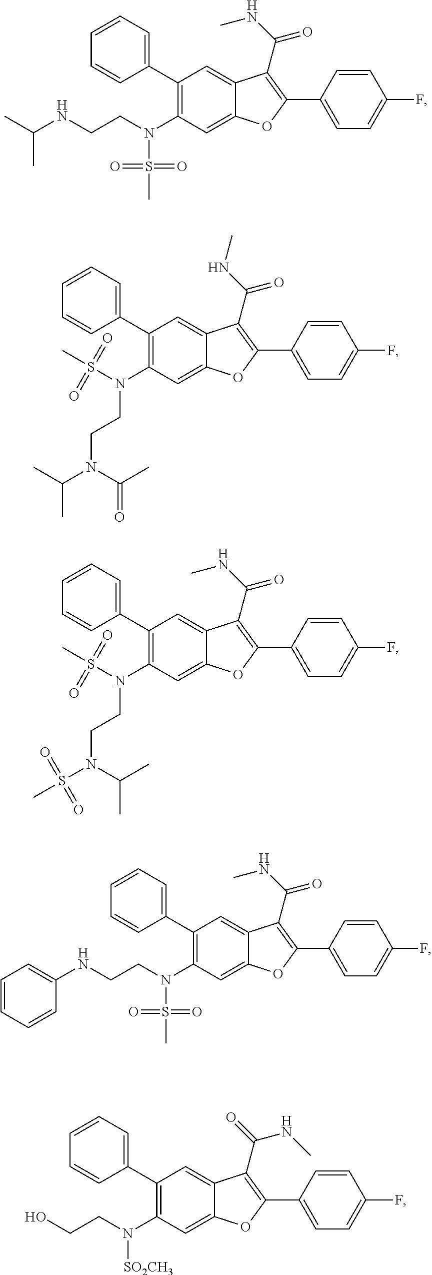 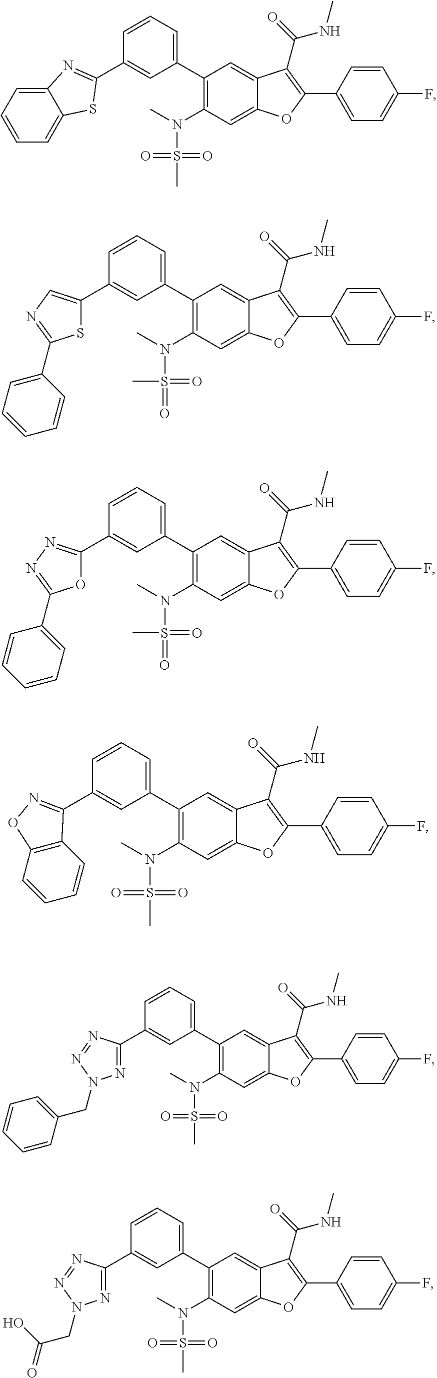 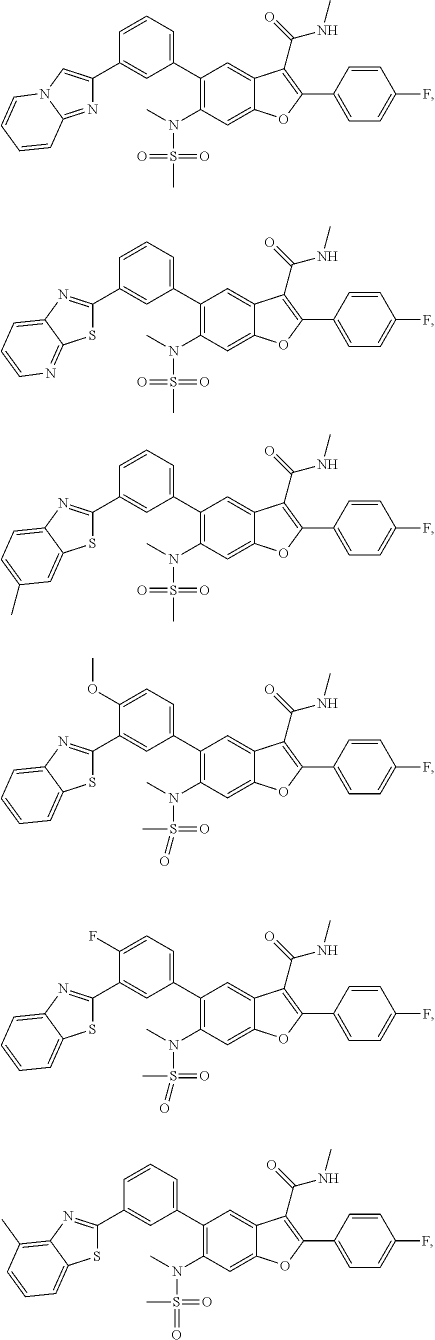 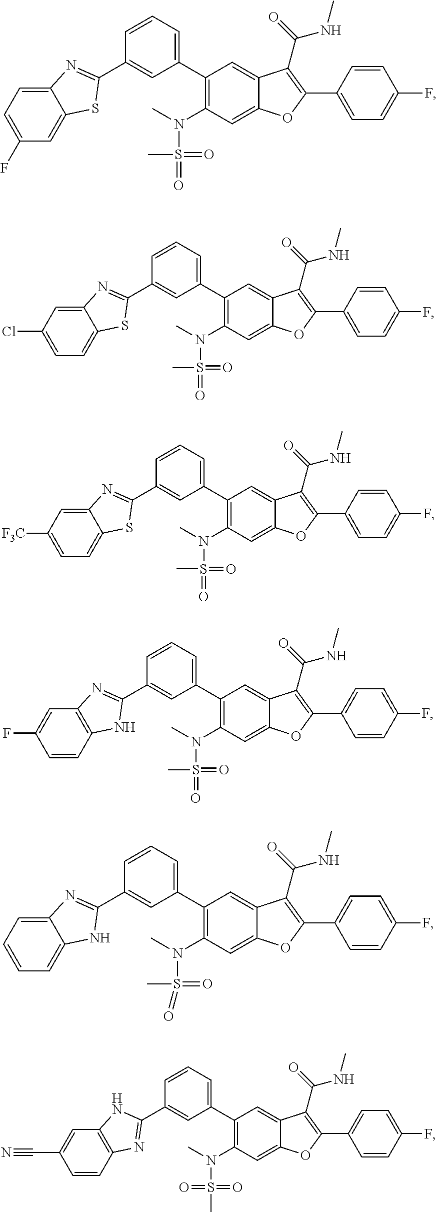 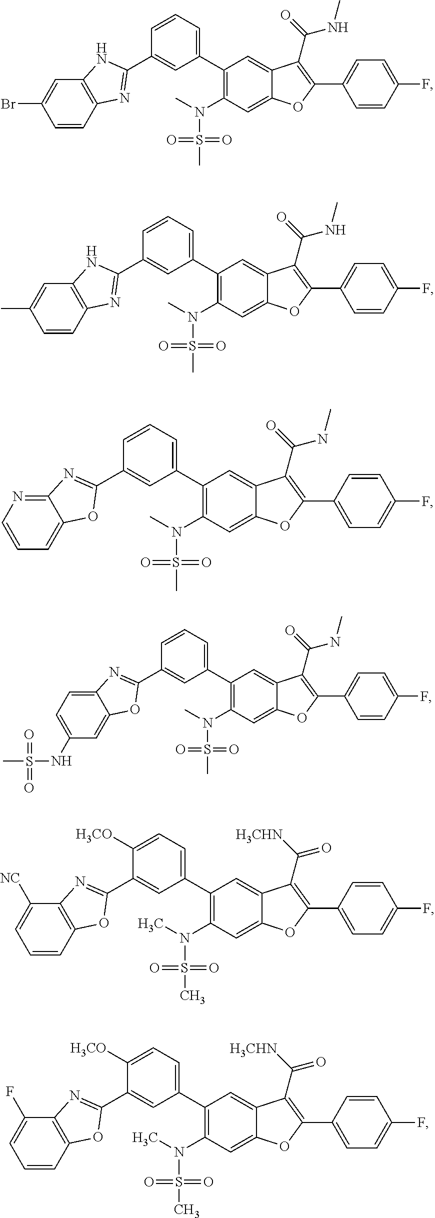 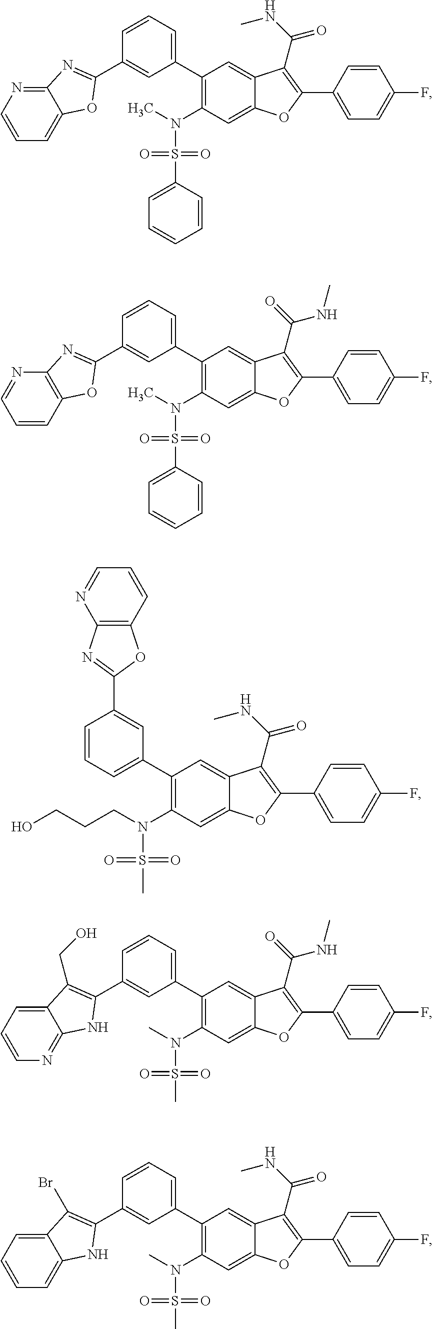 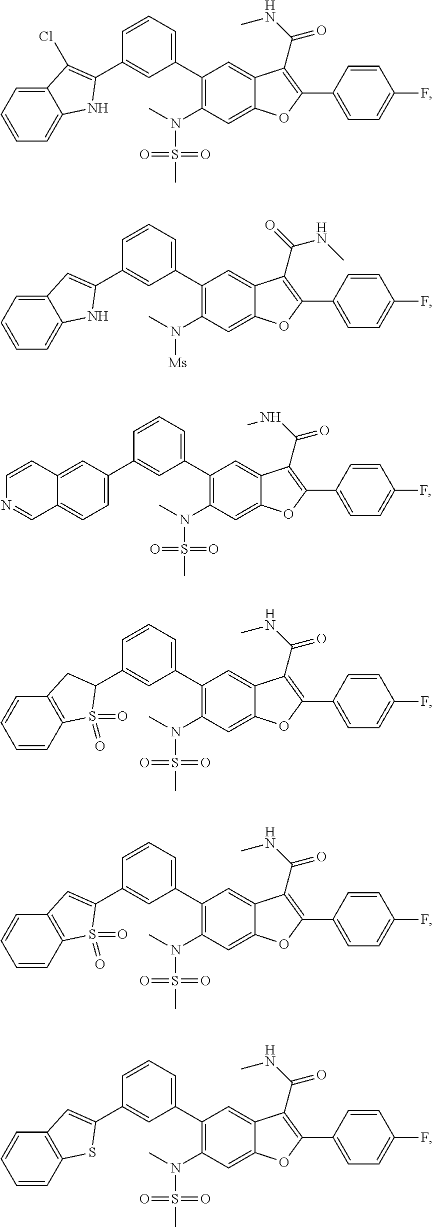 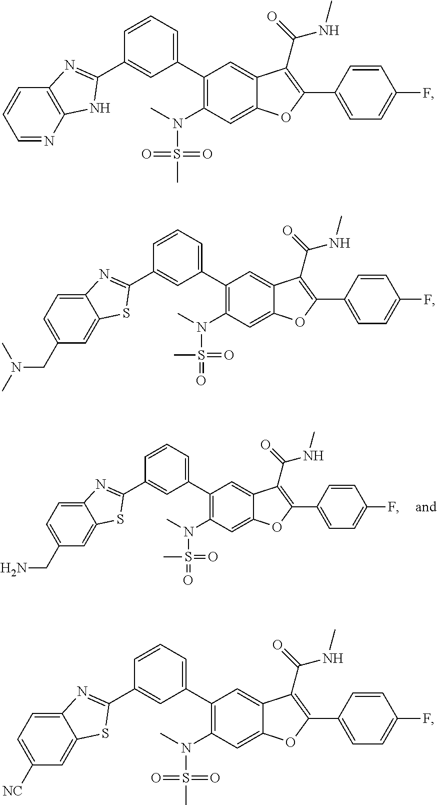
or a pharmaceutically acceptable salt thereof

30. A pharmaceutical composition comprising an effective amount of the compound according to claim 1, and a pharmaceutically acceptable carrier.

31. The pharmaceutical composition according to claim 30, further comprising a second therapeutic agent selected from the group consisting of HCV antiviral agents, immunomodulators, and anti-infective agents.

32. The pharmaceutical composition according to claim 31, wherein said second therapeutic agent is selected from the group consisting of HCV protease inhibitors, HCV NS5A inhibitors and HCV NS5B polymerase inhibitors.

33. A use of the compound according to claim 1, in the preparation of a medicament for inhibiting HCV NS5B activity or for preventing and/or treating infection by HCV in a subject in need thereof.

34. A method of treating a patient infected with HCV comprising the step of administering an amount of the compound according to claim 1 effective to prevent and/or treat infection by HCV in a subject in need thereof.

35. The method according to claim 34, further comprising the step of administering pegylated-interferon alpha and ribavirin.
